SEQUENCE LISTING (2 sequences)

SEQ ID NO: 1 - DNA, 21 bp, Artificial sequence
  Primer; synthetic construct
aagcttatggatggatggaga

SEQ ID NO: 2 - DNA, 21 bp, Artificial sequence
  Primer; synthetic construct
ggatcctcagcgggccaaggc

(19) United States
(12) Patent Application Publication
(10) Pub. No.: US 2010/0121048 A1
(43) Pub. Date: May 13, 2010

(54) Cyclic Amine Compound and Use Thereof for the Prophylaxis or Treatment of Hypertension

(76) Inventors: Takanobu Kuroita, Osaka (JP); Yasuhiro Imaeda, Osaka (JP); Naohiro Taya, Osaka (JP); Tsuneo Oda, Osaka (JP); Kouichi Iwanaga, Osaka (JP); Yasutomi Asano, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/224,046

(22) PCT Filed: Feb. 15, 2007
(86) PCT No.: PCT/JP2007/053242

§ 371 Date: Nov. 6, 2008

Related U.S. Application Data
(60) Provisional application No. 60/774,133, filed on Feb. 16, 2006.

(51) Int. Cl. C07D 403/12; C07D 417/14; C07D 401/14; C07D 403/06; C07D 413/14; C07D 409/06; C07D 403/14

(52) U.S. Cl. 540/524; 544/372; 544/60; 544/364; 544/371; 544/369; 544/121; 544/58.2; 544/379

(57) ABSTRACT

The present invention relates to a compound represented by the formula: (I) wherein ring A is a 5- or 6-membered aromatic heterocycle optionally having substituent (s); U, V and W are each independently C or N, provided that when any one of U, V and W is N, then the others should be C; Ra and Rb are each independently a cyclic group optionally having substituent(s), a C₁₋₁₀alkyl group optionally having substituent(s), a C₂₋₁₀ alkenyl group optionally having substituent(s), or a C₂₋₁₀alkynyl group optionally having substituent(s); X is a bond, or a spacer having 1 to 6 atoms in the main chain; Y is a spacer having 1 to 6 atoms in the main chain; Rc is a hydrocarbon group optionally containing heteroatom(s) as the constituting atom(s), which optionally has substituent(s); m and n are each independently 1 or 2; and ring B optionally further has substituent(s), or a salt thereof. The compound of the present invention has excellent renin inhibitory activity, and thus is useful as an agent for the prophylaxis or treatment of hypertension, various organ damages attributable to hypertension, and the like.

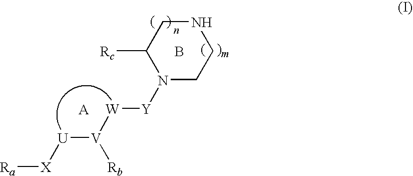

### TECHNICAL FIELD

The present invention relates to a cyclic amine compound which has excellent renin inhibitory activity and is useful as an agent for the prophylaxis or treatment of hypertension, various organ damages attributable to hypertension, and the like.

### BACKGROUND OF THE INVENTION

Hypertension is one of representative lifestyle-related diseases. Hypertension which is left untreated for long time lays a heavy burden on the cardiovascular system and results in arteriosclerosis to progress, thus causing various disorders in important organs, such as cerebral hemorrhage, cerebral infarction, cardiac failure, angina pectoris, myocardial infarction, renal failure and the like. Accordingly, the purpose of treating hypertension lies not only in lowering the blood pressure, but also in improving and/or preventing disorders in important organs including brain, heart and kidney, by controlling the blood pressure. As a method of treating hypertension, there are available fundamental treatments based on improvement in the lifestyle, such as dietetic therapy, exercise therapy and the like, as well as an attempt to control the blood pressure by positive pharmaceutical intervention.

The renin-angiotensin (RA) system is a system of biosynthesis of angiotensin II (AII), which is a major vasopressor factor, and takes an important role in the control of the blood pressure and the amount of body fluid. AII exhibits a strong vasoconstrictive effect brought by the intervention of AII receptors on the cellular membrane, thus raising the blood pressure, and also promotes cellular propagation or production of extracellular matrix by directly acting on the AII receptors in the cardiac cells or renal cells. Therefore, drugs inhibiting increase in the activity of the RA system can be expected to have a blood pressure lowering action as well as a powerful organ protecting action, and thus active researches on such drugs have been conducted so far.

The method of inhibiting the AII action is broadly classified into methods of inhibiting the biosynthesis of AII and methods of inhibiting the binding of AII to AII receptors. For the drugs inhibiting the biosynthesis of AII, angiotensin converting enzyme (ACE) inhibitory drugs have been already put to practical use and are being confirmed to have a blood pressure lowering action as well as an effect for protecting various organs. However, since ACE is an enzyme identical to kininase II, which is a bradykinin degrading enzyme, ACE inhibitory drug inhibits the biosynthesis of AII as well as the degradation of bradykinin. As a result, ACE inhibitory action is believed to induce side effects such as dry cough, angioedema and the like, which are considered to be caused by accumulation of bradykinin.

As the drugs inhibiting the binding of AII to AII receptors, AII type 1 receptor blockers (ARB) have been developed. ARB has a merit in that it can inhibit, at the receptor level, not only ACE but also the action of AII that is biosynthesized by an enzyme other than ACE, such as chymase or the like. It is known that administration of ACE inhibitors and ARB increases the plasma renin activity (PRA) as a compensatory feedback effect, since these drugs act on a more peripheral region of the RA system.

Renin is an enzyme occupying a position at the uppermost stream of the RA system, and converts angiotensinogen to angiotensin I. A renin inhibitory drug inhibits the RA system by inhibiting the biosynthesis of AII in the same manner as the ACE inhibitory drugs do, and thus can be expected to have a blood pressure lowering action or an effect of protecting various organs. Since the renin inhibitory drug does not have influence on the metabolism of bradykinin, it is believed to have no risk of side effects such as dry cough and the like, that are observed with the ACE inhibitory drugs. Furthermore, while the ACE inhibitory drugs or ARB increase the PRA level, the renin inhibitory drugs are the only drugs that can reduce PRA.

Investigation for the renin inhibitory drugs was started earliest among the RA system inhibitory drugs. However, when anti-renin antibodies or renin inhibitory peptides are put aside, development of orally administrable low molecular weight drugs has not yet been achieved, and only recently, clinical tests for orally administrable Aliskiren are being in progress (See, for example, Chemistry and Biology (Chem. Biol.), Vol. 7, pp. 493-504 (2000); Hypertension, Vol. 42, pp. 1137-1143 (2003); and Journal of Hypertension (J. Hypertens.), Vol. 23, pp. 417-426 (2005)). In addition to that, low molecular weight renin inhibitory drugs are disclosed in WO 2004/002957 and WO 2004/089915.

Moreover, several compounds having structures that are similar to that of the cyclic amine derivative of the present invention are known (See, for example, WO 2003/002559, WO 2003/002561, WO 2003/032991, WO 2003/041711, WO 2003/051368, WO 2003/051871, WO 2003/051873, WO 2004/026866, WO 2004/041791, and WO 2004/041807). However, these compounds are all orexin receptor antagonists and are different from the compound of the present invention which is a renin inhibitory drug.

### DISCLOSURE OF THE INVENTION

The present invention provides a novel cyclic amine compound which has a chemical structure different from the structures of the aforementioned known compounds, has excellent renin inhibitory activity, and thus can be sufficiently put to practical use as a medicine.

The present inventors have conducted various studies, and as a result, found that a compound represented by the following formula (I), characterized by a chemical structure in which the ring A-constituting atom (U) to which X is bonded and the ring A-constituting atom (V) to which Rb is bonded are adjacent to each other, and the ring A-constituting atom (V) to which Rb is bonded and the ring A-constituting atom (W) to which Y is bonded are adjacent to each other; and a compound represented by the following formula (I′), characterized by a chemical structure in which the ring A-constituting atom (U) to which R is bonded and the ring A-constituting atom (V) to which R′ is bonded are adjacent to each other, and the ring A-constituting atom (V) to which R′ is bonded and the ring A-constituting atom (W) to which Y is bonded are adjacent to each other, have excellent renin inhibitory activities and can be sufficiently put to practical use as medicines, thereby completing the invention.

Accordingly, the present invention relates to the following:
[1] A compound represented by the formula:

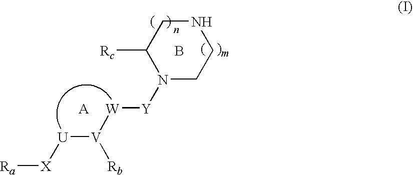

wherein
ring A is a 5- or 6-membered aromatic heterocycle optionally having substituent(s);
U, V and W are each independently C or N, provided that when any one of U, V and W is N, then the others should be C;
Ra and Rb are each independently a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
X is a bond, or a spacer having 1 to 6 atoms in the main chain;
Y is a spacer having 1 to 6 atoms in the main chain;
Rc is a hydrocarbon group optionally containing heteroatom(s) as the constituting atom(s), which optionally has substituent(s);
m and n are each independently 1 or 2; and
ring B optionally further has substituent(s),
or a salt thereof (hereinafter to be abbreviated as compound (I)).
[2] The compound of the aforementioned [1], wherein ring A is a 5-membered aromatic heterocycle optionally having substituent(s).
[3] The compound of the aforementioned [1], wherein ring A is imidazole or pyrrole, each of which optionally has substituent(s).
[4] The compound of the aforementioned [1], wherein Ra and Rb are each independently a cyclic group optionally having substituent(s).
[5] The compound of the aforementioned [1], wherein Ra and Rb are each independently a C₆₋₁₄ aryl group optionally having substituent(s), a 5- or 6-membered non-aromatic heterocyclic group optionally having substituent(s), or a C₃₋₁₀ cycloalkyl group condensed with a benzene ring, which optionally has substituent(s).
[6] The compound of the aforementioned [1], wherein Ra and Rb are each independently a C₆₋₁₄ aryl group optionally having substituent(s), or a C₃₋₁₀ cycloalkyl group condensed with a benzene ring, which optionally has substituent(s).
[7] The compound of the aforementioned [1], wherein Ra is a phenyl group optionally having substituent(s), an indanyl group optionally having substituent(s) or a piperidinyl group optionally having substituent(s).
[8] The compound of the aforementioned [1], wherein Rb is a phenyl group optionally having substituent(s).
[9] The compound of the aforementioned [1], wherein X is a bond or a straight chain C₁₋₆ alkylene group optionally having substituent(s).
[10] The compound of the aforementioned [1], wherein X is a bond, or a group represented by the formula: —(R¹)C(R²)— (wherein R¹ and R² are each independently a hydrogen atom or a C₁₋₃ alkyl group).
[11] The compound of the aforementioned [1], wherein X is a bond.
[12] The compound of the aforementioned [1], wherein Y is —CO—, —CH₂—, —CH₂CO— or —SO₂—.
[13] The compound of the aforementioned [1], wherein Y is —CO—.
[14] The compound of the aforementioned [1], wherein Rc is
1) a group represented by the formula:

R³—(Z₁)q-(Z)p-

wherein
R³ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₁ is —CO—, —O—, —S—, —S(O)— or —S(O)₂—; and
p and q are each independently 0 or 1;
2) a group represented by the formula:

R⁴—Z₂—(R⁵)C(R⁶)—(Z)p-

wherein
R⁴ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
R⁵ and R⁶ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s), or R⁵ and R⁶ in combination form an oxo group;
Z is a C₁₋₄ alkylene group;
Z₂ is —O—, or a group represented by the formula: —N(R⁷)— (wherein R⁷ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s));
p is 0 or 1; and
when Z₂ is a group represented by the formula: —N(R⁷)—, then R⁴ and R⁷ are optionally bonded to each other to form, together with the adjacent nitrogen atom, a nitrogen-containing heterocycle optionally having substituent(s);
3) a group represented by the formula:

R⁸—Z₃—N(R⁹)—(Z)p-

wherein
R⁸ and R⁹ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₃ is —CO—, —CONH— or —SO₂—; and
p is 0 or 1;
4) a group represented by the formula:

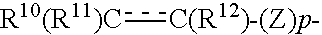

wherein
R¹⁰, R¹¹ and R¹² are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
[symbol] is a single bond or a double bond; and
p is 0 or 1; or
5) a group represented by the formula:

R¹³O—N═C(R¹⁴)—(Z)p-

wherein
R¹³ and R¹⁴ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄, alkylene group; and
p is 0 or 1.
[15] The compound of the aforementioned [1], wherein Rc is a group represented by the formula:

R³—(Z₁)q-(Z)p-

wherein
R³ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z¹ is —CO—, —O—, —S—, —S(O)— or —S(O)₂—; and
p and q are each independently 0 or 1.
[16] The compound of the aforementioned [1], wherein Rc is a group represented by the formula:

R⁴—Z₂—(R⁵)C(R⁶)—(Z)p-

wherein
R⁴ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
R⁵ and R⁶ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s), or R⁵ and R⁶ in combination form an oxo group;
Z is a C₁₋₄ alkylene group;
Z₂ is —O—, or a group represented by the formula: —N(R⁷)— (wherein R⁷ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s));
p is 0 or 1; and
when Z₂ is a group represented by the formula: —N(R⁷)—, then R⁴ and R⁷ are optionally bonded to each other to form, together with the adjacent nitrogen atom, a nitrogen-containing heterocycle optionally having substituent(s).
[17] The compound of the aforementioned [1], wherein Rc is a group represented by the formula:

R⁸—Z₃—N(R⁹)—(Z)p-

wherein
R⁸ and R⁹ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₃ is —CO—, —CONH— or —SO₂—; and
p is 0 or 1.
[18] The compound of the aforementioned [1], wherein Rc is a C₁₋₆ alkyl group optionally substituted by 1 to 3 substituents selected from

(i) an optionally substituted C₆₋₁₄ aryl group, and

(ii) an optionally substituted C₁₋₆ alkoxy group.
[19] The compound of the aforementioned [1], wherein both m and n are 1.
[20] The compound of the aforementioned [1], wherein the compound represented by the formula (I) is a compound selected from the group consisting of
(2R)-2-benzyl-1-{[1-(2,3-dimethoxyphenyl)-5-methyl-2-phenyl-1H-pyrrol-3-yl]carbonyl)piperazine,
4-[3-(4-{[(2R)-2-benzylpiperazin-1-yl]carbonyl}-5-phenyl-1H-imidazol-1-yl)phenyl]morpholine,
(2R)-2-benzyl-1-{[1-(2,3-dimethoxyphenyl)-5-phenyl-1H-imidazol-4-yl]carbonyl}piperazine,
(2R)-2-benzyl-1-{[1-(2,3-dihydro-1H-inden-2-yl)-5-phenyl-1H-imidazol-4-yl]carbonyl}piperazine,
2-{3-[(2-benzylpiperazin-1-yl)carbonyl]-2-phenyl-1H-pyrrol-1-yl}-N-butylaniline,
4-{3-(3-{[(2R)-2-benzylpiperazin-1-yl]carbonyl}-5-methyl-2-phenyl-1H-pyrrol-1-yl)phenyl]morpholine,
4-[((2R)-1-{[1-(3-morpholinophenyl)-5-phenyl-1H-imidazol-4-yl]carbonyl}piperazin-2-yl)methylbenzoyl acid,
4-(3-(4-{[(2S)-2-({[4-(methylsulfonyl)benzyl]oxy}methyl)piperazin-1-yl]carbonyl}-5-phenyl-1H-imidazol-1-yl)phenyl]morpholine,
(2R)-2-benzyl-1-[(2-methoxy-1,5-diphenyl-1H-imidazol-4-yl)carbonyl]piperazine,
(2R)-2-benzyl-1-({5-phenyl-1-[1-(phenylsulfonyl)piperidin-3-yl]-1H-imidazol-4-yl}carbonyl)piperazine,
(2R)-2-benzyl-1-[(1-{1-[(6-methoxypyridin-3-yl)sulfonyl]piperidin-3-yl}-5-phenyl-1H-imidazol-4-yl)carbonyl]piperazine, and
4-[(3S)-3-(4-{[(2R)-2-benzylpiperazin-1-yl]carbonyl}-5-phenyl-1H-imidazol-1-yl)-5-phenylpentanoyl]morpholine.
[21] A prodrug of the compound of the aforementioned [1].
[22] A medicine comprising the compound of the aforementioned [1] or a salt thereof, or a prodrug thereof.
[23] The medicine of the aforementioned [22], which is a renin inhibitory drug.
[24] The medicine of the aforementioned [22], which is an agent for the prophylaxis or treatment of hypertension.
[25] The medicine of the aforementioned [22], which is an agent for the prophylaxis or treatment of various organ damages attributable to hypertension.
[26] A renin inhibitory drug comprising a compound represented by the formula:

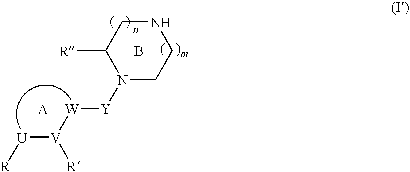

wherein
ring A is an aromatic heterocycle optionally having substituent(s);
U, V and W are each independently C or N, provided that when any one of U, V and W is N, then the others should be C;
R, R′ and R″ are each independently a substituent;
Y is a spacer having 1 to 6 atoms In the main chain;
m and n are each independently 1 or 2; and
ring B optionally further has substituent(s),
or a salt thereof (hereinafter to be abbreviated as compound (I′)), or a prodrug thereof.

The cyclic amine compound of the present invention has excellent renin inhibitory activity, and thus is useful as an agent for the prophylaxis or treatment of hypertension, various organ damages attributable to hypertension, and the like.

Unless otherwise specified, the “halogen atom” as used in the present specification means a fluorine atom, a chlorine atom, a bromine atom or an iodine atom.

Unless otherwise specified, the “C₁₋₄ alkylenedioxy group” as used in the present specification means methylenedioxy, ethylenedioxy, trimethylenedioxy or the like.

Unless otherwise specified, the “C₁₋₆ alkyl group” as used in the present specification means methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl or the like.

Unless otherwise specified, the “C₁₋₆ alkoxy group” as used in the present specification means methoxy, ethoxy, propoxy, isopropoxy, butoxy, isobutoxy, sec-butoxy, tert-butoxy or the like.

Unless otherwise specified, the “C₁₋₆ alkoxy-carbonyl group” as used in the present specification means methoxycarbonyl, ethoxycarbonyl, propoxycarbonyl, tert-butoxycarbonyl or the like.

Unless otherwise specified, the “C₁₋₆ alkyl-carbonyl group” as used in the present specification means acetyl, propanoyl, butanoyl, isobutanoyl, pentanoyl, isopentanoyl, hexanoyl or the like.

Each symbol in the formulas (I) and (I′) is described in detail in the following.

Ra and Rb are each independently a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s).

As the “C₁₋₁₀ alkyl group” of the “C₁₋₁₀ alkyl group optionally having substituent(s)” for Ra or Rb, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned. Among these, a C₁₋₆ alkyl group is preferred.

As the “C₂₋₁₀ alkenyl group” of the “C₂₋₁₀ alkenyl group optionally having substituent(s)” for Ra or Rb, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned. Among these, a C₂₋₆ alkenyl group is preferred.

As the “C₂₋₁₀ alkynyl group” of the “C₂₋₁₀ alkynyl group optionally having substituent(s)” for Ra or Rb, for example, ethynyl, 1-propynyl, 2-propynyl, 1-butynyl, 2-butynyl, 3-butyryl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned. Among these, a C₂₋₆ alkynyl group is preferred.

These “C₁₋₁₀ alkyl group”, “C₂₋₁₀ alkenyl group” and “C₂₋₁₀ alkynyl group” optionally have substituent(s) (preferably, 1 to 3 substituents) at substitutable position(s). When the number of the substituents is not less than 2, respective substituents may be the same or different.

As such substituents, for example,
(1) a C₃₋₁₀ cycloalkyl group (e.g., cyclopropyl, cyclohexyl);
(2) a C₆₋₁₄ aryl group (e.g., phenyl, naphthyl) optionally substituted by 1 to 3 substituents selected from

(i) a carboxyl group,

(ii) a hydroxy group,

(iii) a C₁₋₆ alkyl group optionally substituted by 1 to 3 substituents selected from
(a) a hydroxy group, and
(b) a halogen atom,

(iv) a C₁₋₆ alkoxy group optionally substituted by 1 to 3 substituents selected from
(a) a C₁₋₆ alkoxy group,
(b) a carbamoyl group optionally mono- or di-substituted by substituent(s) selected from a C₁₋₆ alkyl group optionally substituted by a carbamoyl group, and a C₁₋₆ alkylsulfonyl group;
(c) a carboxyl group,
(d) a C₁₋₆ alkoxy-carbonyl group optionally substituted by a non-aromatic heterocyclic group (e.g., dioxolyl) optionally substituted by 1 to 3 substituents selected from an oxo group and a C₁₋₆ alkyl group,
(e) a, cyano group, and
(f) a non-aromatic heterocyclic group (e.g., oxadiazolinyl) optionally substituted by an oxo group,

(v) a carbamoyl group optionally mono- or di-substituted by substituent(s) selected from
(a) a C₁₋₆ alkyl group optionally substituted by a hydroxy group, and
(b) a C₁₋₆ alkylsulfonyl group,

(vi) a non-aromatic heterocyclic group (e.g., oxadiazolinyl) optionally-substituted by an oxo group,

(vii) an aromatic heterocyclic group (e.g., tetrazolyl),

(viii) a C₁₋₆ alkoxy-carbonyl group optionally substituted by a non-aromatic heterocyclic group (e.g., dioxolyl) optionally substituted by 1 to 3 substituents selected from an oxo group and a C₁₋₆ alkyl group,

(xi) a cyano group,

(x) a sulfamoyl group,

(xi) a halogen atom,

(xii) a C₁₋₆ alkylsulfonyl group (e.g., methylsulfonyl), and

(xiii) a C₁₋₆ alkyl sulfonyloxy group (e.g., methylsulfonyloxy);
(3) an aromatic heterocyclic group (e.g., thienyl, furyl, pyridyl, oxazolyl, thiazolyl, tetrazolyl, oxadiazolyl, pyrazinyl, quinolyl, indolyl, imidazolyl, benzimidazolyl) optionally substituted by 1 to 3 substituents selected from

(i) a C₁₋₆ alkyl group optionally substituted by 1 to 3 halogen atoms,

(ii) a hydroxy group,

(iii) a C₁₋₆ alkoxy group,

(iv) a halogen atom, and

(v) a C₆₋₁₄ aryl group (e.g., phenyl);
(4) a non-aromatic heterocyclic group (the non-aromatic heterocyclic group may be oxidized; e.g., tetrahydrdfuryl, morpholinyl, thiomorpholinyl, piperidinyl, pyrrolidinyl, piperazinyl, dioxolyl, dioxolanyl, 1,3-dihydro-2-benzofuranyl, thiazolidinyl, oxadiazolyl, 1-oxidothibmorpholinyl, 1,1-dioxidothiomorpholinyl, tetrahydropyranyl) optionally substituted by 1 to 3 substituents selected from

(i) a C₁₋₆ alkyl group optionally substituted by 1 to 3 substituents selected from
(a) a halogen atom,
(b) a hydroxy group,
(c) a C₆₋₁₄ aryl group (e.g., phenyl),
(d) a C₁₋₆ alkoxy group, and
(e) a non-aromatic heterocyclic group (the non-aromatic heterocyclic group may be oxidized; e.g., tetrahydrofuryl) optionally substituted by a C₁₋₆ alkyl group,

(ii) a hydroxy group,

(iii) a C₁₋₆ alkoxy group,

(iv) a C₁₋₆ alkyl-carbonyl group optionally substituted by an amino group optionally mono- or di-substituted by a C₁₋₆ alkyl-carbonyl group,

(v) a C₁₋₆ alkoxy-carbonyl group,

(vi) a carboxyl group,

(vii) a carbamoyl group optionally mono- or di-substituted by a C₁₋₆ alkyl group optionally substituted by 1 to 3 substituents selected from a hydroxy group and a carbamoyl group,

(viii) an oxo group,

(ix) a halogen atom,

(x) a C₆₋₁₄ aryl-carbonyl group (e.g., benzoyl),

(xi) a C₁₋₆ alkylsulfonyl group, and

(xii) a C₆₋₁₄ arylsulfonyl group (e.g., phenylsulfonyl);
(5) an amino group optionally mono- or di-substituted by substituent(s) selected from

(i) a C₁₋₁₀ alkyl group optionally substituted by 1 to 3 substituents selected from
(a) a hydroxy group,
(b) a alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group,
(i) an amino group optionally mono- or di-substituted by a C₁₋₆ alkoxy-carbonyl group optionally substituted by a C₆₋₁₄ aryl group (e.g., phenyl), and
(j) a carbamoyl group,

(ii) a C₁₋₆ alkyl-carbonyl group optionally substituted by 1 to 3 substituents selected from
(a) a carboxyl group,
(b) a C₆₋₁₄ aryl group (e.g., phenyl),
(c) an amino group optionally mono- or di-substituted by a C₁₋₆ alkyl-carbonyl group,
(d) a C₁₋₆ alkoxy group optionally substituted by a C₁₋₆ alkoxy group,
(e) an aromatic heterocyclic group (e.g., thienyl),
(f) a C₁₋₆ alkoxy-carbonyl group,
(g) a carbamoyl group optionally mono- or di-substituted by a C₃₋₁₀ cycloalkyl group, and
(h) a non-aromatic heterocyclylcarbonyl group (e.g., morpholinylcarbonyl),

(iii) a C₁₋₆ alkoxy-carbonyl group optionally substituted by a C₆₋₁₄ aryl group (e.g., phenyl),

(iv) a C₆₋₁₄ aryl-carbonyl group (e.g., benzoyl) optionally substituted by a C₁₋₆ alkoxy group,

(v) a C₇₋₁₃ aralkyl-carbonyl group (e.g., benzylcarbonyl, phenethylcarbonyl),

(vi) a carbamoyl group optionally mono- or di-substituted by a C₁₋₆ alkyl group optionally substituted by 1 to 3 substituents selected from
(a) a carboxyl group,
(b) a C₁₋₆ alkoxy-carbonyl group, and
(c) a carbamoyl group,

(vii) a C₆₋₁₄ aryl-carbamoyl group (e.g., phenylcarbamoyl, 1-naphthylcarbamoyl, 2-naphthylcarbamoyl),

(viii) a C₇₋₁₃ aralkyl-carbamoyl group (e.g., benzylcarbamoyl),

(ix) a C₁₋₆ alkylsulfonyl group (e.g., methylsulfonyl, ethylsulfonyl, isopropylsulfonyl),

(x) a C₆₋₁₄ arylsulfonyl group (e.g., benzenesulfonyl, toluenesulfonyl, 1-naphthalenesulfonyl, 2-naphthalenesulfonyl),

(xi) a C₇₋₁₃ aralkylsulfonyl group (e.g., benzylsulfonyl),

(xii) a non-aromatic heterocyclic group (the non-aromatic heterocyclic group may be oxidized; e.g., tetrahydrofuryl, tetrahydropyranyl) optionally substituted by a hydroxy group,

(xiii) a C₆₋₁₄ aryl group (e.g., phenyl), and

(xiv) a C₃₋₁₀ cycloalkyl-carbonyl group;
(6) an amidino group;
(7) a C₁₋₆ alkyl-carbonyl group optionally substituted by 1 to 3 substituents selected from a halogen atom and a hydroxy group;
(8) a C₁₋₆ alkoxy-carbonyl group optionally substituted by 1 to 3 substituents selected from a halogen atom and a C₆₋₁₄ aryl group (e.g., phenyl);
(9) a C₁₋₆ alkylsulfonyl group (e.g., methylsulfonyl) optionally substituted by 1 to 3 halogen atoms;
(10) a carbamoyl group optionally mono- or di-substituted by substituent(s) selected from

(i) a C₁₋₆ alkyl group optionally substituted by 1 to 3 substituents selected from a halogen atom, a hydroxy group, a carbamoyl group and an aromatic heterocyclic group (e.g., furyl),

(ii) a C₆₋₁₄ aryl group (e.g., phenyl),

(iii) a C₇₋₁₃ aralkyl group (e.g., benzyl), and

(iv) an aromatic heterocyclyl-C₁₋₆ alkyl group (e.g., furfuryl);
(11) a thiocarbamoyl group optionally mono- or di-substituted by a C₁₋₆ alkyl group optionally substituted by 1 to 3 halogen atoms;
(12) a sulfamoyl group optionally mono- or di-substituted by a C₁₋₆ alkyl group optionally substituted by 1 to 3 halogen atoms;
(13) a carboxyl group;
(14) a hydroxy group;
(15) a C₁₋₆ alkoxy group optionally substituted by 1 to 3 substituents selected from

(i) a halogen atom,

(ii) a carboxyl group,

(iii) a hydroxy group,

(iv) a C₁₋₆ alkoxy group,

(v) a C₆₋₁₄ aryl group (e.g., phenyl) optionally substituted by a C₁₋₆ alkylsulfonyl group,

(vi) a C₁₋₆ alkoxy-carbonyl group,

(vii) a C₁₋₆ alkylsulfonyl group (e.g., methylsulfonyl),

(viii) a non-aromatic heterocyclic group (the non-aromatic heterocyclic group may be oxidized; e.g., 1,1-dioxidothiomorpholinyl, imidazolidinyl, oxetanyl) optionally substituted by 1 to 3 substituents selected from a C₁₋₆ alkyl group and an oxo group, and

(ix) a carbamoyl group optionally mono- or di-substituted by a C₁₋₆ alkyl group optionally substituted by 1 to 3 substituents selected from a carbamoyl group and a hydroxy group;
(16) a C₂₋₆ alkenyloxy group (e.g., ethenyloxy) optionally substituted by 1 to 3 halogen atoms;
(17) a C₃₋₁₀ cycloalkyloxy group (e.g., cyclohexyloxy);
(18) a C₇₋₁₃ aralkyloxy group. (e.g., benzyloxy);
(19) a C₆₋₁₄ aryloxy group (e.g., phenyloxy, naphthyloxy) optionally substituted by 1 to 3 substituents selected from

(i) a halogen atom,

(ii) a carboxyl group,

(iii) a carbamoyl group,

(iv) a C₁₋₆ alkyl group optionally substituted by 1 to 3 substituents selected from a carboxyl group and a halogen atom,

(v) a C₁₋₄ alkylenedioxy group,

(vi) a C₁₋₆ alkyl-carbonyl group, and

(vii) a cyano group;
(20) a non-aromatic heterocyclyloxy group (the non-aromatic heterocycle may be oxidized; e.g., tetrahydrothiopyranyloxy, 1-oxidotetrahydrothiopyranyloxy, 1,1-dioxidotetrahydrothiopyranyloxy);
(21) a C₁₋₆ alkyl-carbonyloxy group (e.g., acetyloxy, tert-butylcarbonyloxy);
(22) a mercapto group;
(23) a C₁₋₆ alkylthio group (e.g., methylthio, ethylthio) optionally substituted by 1 to 3 halogen atoms;
(24) a C₇₋₂₀ aralkylthio group (e.g., benzylthio, tritylthio);
(25) a C₆₋₁₄ arylthio group (e.g., phenylthio, naphthylthio);
(26) a sulfo group;
(27) a cyano group;
(28) an azido group;
(29) a nitro group;
(30) a nitroso group;
(31) a halogen atom;
(32) a C₁₋₆ alkylsulfinyl group (e.g., methylsulfinyl);
(33) an oxo group;
(34) a C₃₋₁₀ cycloalkyl-C₁₋₆ alkyloxy group (e.g., cyclopropylmethyloxy);
(35) a non-aromatic heterocyclylcarbonyl group (the non-aromatic heterocycle may be oxidized; e.g., morpholinylcarbonyl, piperazinylcarbonyl) optionally substituted by C₁₋₆ alkyl group optionally substituted by a C₆₋₁₄ aryl group (e.g., phenyl);
(36) a C₁₋₄ alkylenedioxy group optionally substituted by 1 to 3 halogen atoms;
(37) a hydroxyimino group optionally substituted by a C₁₋₆ alkyl group;
(38) a C₆₋₁₄ arylsulfonyl group (e.g., phenylsulfonyl) optionally substituted by a C₁₋₆ alkoxy group;
(39) a C₃₋₁₀ cycloalkylsulfonyl group (e.g., cyclopropylsulfonyl);
(40) an aromatic heterocyclylsulfonyl group (e.g., pyridylsulfonyl, pyrazolylsulfonyl, thienylsulfonyl, furylsulfonyl, imidazolylsulfonyl) optionally substituted by 1 to 3 substituents selected from

(i) a C₁₋₆ alkyl group,

(ii) a C₁₋₆ alkoxy group,

(iii) a C₁₋₆ alkoxy-carbonyl group, and

(iv) a halogen atom;
(41) a C₆₋₁₄ arylsulfinyl group (e.g., phenylsulfinyl); and the like can be mentioned.

As the “cyclic group” of the “cyclic group optionally having substituent(s)” for Ra or Rb, for example, an aromatic group, a non-aromatic cyclic group and the like can be mentioned.

As the aromatic group, for example, an aromatic hydrocarbon group, an aromatic heterocyclic group and the like can be mentioned.

As the aromatic hydrocarbon group, for example, a C₆₋₁₄ aryl group and the like can be mentioned.

As the C₆₋₁₄ aryl group, for example, phenyl, naphthyl, anthryl, phenanthryl, acenaphthylenyl, biphenylyl and the like can be mentioned. Among these, phenyl and naphthyl are preferred.

As the aromatic heterocyclic group, for example, a 4- to 7-membered (preferably 5- or 6-membered) monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group can be mentioned. As the fused aromatic heterocyclic group, for example, a group derived from a fused ring wherein a ring constituting such 4- to 7-membered monocyclic aromatic heterocyclic group, and 1 or 2 rings selected from a 5- or 6-membered aromatic heterocycle containing 1 or 2 nitrogen atoms, a 5-membered aromatic heterocycle containing one sulfur atom and a benzene ring are condensed, and the like can be mentioned.

As preferable examples of the aromatic heterocyclic group,
monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl. (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,2,4-thiadiazol-5-yl, 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,3,5-triazin-2-yl, 1,3,5-triazin-4-yl, 1,2,3-triazin-4-yl, 1,2,4-triazin-3-yl) and the like;
fused aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuranyl (e.g., 2-benzofuranyl, 3-benzofuranyl), benzothienyl (e.g., 2-benzothienyl, 3-benzothienyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]Pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl); pyrazolotriazinyl (e.g., pyrazolo[5,1-c][1,2,4]triazin-3-yl) and the like;
   and the like can be mentioned.

As the non-aromatic cyclic group, for example, a non-aromatic cyclic hydrocarbon group, a non-aromatic heterocyclic group and the like can be mentioned.

As the non-aromatic cyclic hydrocarbon group, for example, a C₃₋₁₀ cycloalkyl group, a C₃₋₁₀ cycloalkenyl group and a C₄₋₁₀ cycloalkadienyl group, each of which is optionally condensed with a benzene ring, and the like can be mentioned.

As the C₃₋₁₀ cycloalkyl group, for example, cyclopropyl, cyclobutyl, cyclopentyl, cyclohexyl, cycloheptyl, cyclooctyl, bicyclo[2.2,1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned.

The aforementioned C₃₋₁₀ cycloalkyl group, C₃₋₁₀ cycloalkenyl group and C₄₋₁₀ cycloalkadienyl group are each optionally condensed with a benzene ring, and as such a fused ring group, for example, indanyl, dihydronaphthyl, tetrahydronaphthyl, fluorenyl and the like can be mentioned.

As the non-aromatic heterocyclic group, for example, a 4- to 7-membered (preferably 5- or 6-membered) monocyclic non-aromatic heterocyclic group containing, as a ring-constituting atom besides carbon atoms, 1 to 4 heteroatoms selected from an oxygen atom, a sulfur atom and a nitrogen atom, and a fused non-aromatic heterocyclic group can be mentioned. As the fused non-aromatic heterocyclic group, for example, a group derived from a fused ring wherein a ring constituting such 4- to 7-membered monocyclic non-aromatic heterocyclic group, and 1 or 2 rings selected from a 5- or 6-membered heterocycle containing 1 or 2 nitrogen atoms, a 5-membered heterocycle containing one sulfur atom and a benzene ring are condensed, and the like can be mentioned.

As preferable examples of the non-aromatic heterocyclic group,
monocyclic non-aromatic heterocyclic groups such as pyrrolidinyl (e.g., 1-pyrrolidinyl, 2-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleneiminyl (e.g., hexamethyleneimin-1-yl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tptrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidotetrahydrothiopyranyl (e.g., 1-oxidotetrahydrothiopyran-4-yl), 1,1-dioxidotetrahydrothiopyranyl (e.g., 1,1-dioxidotetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl) and the like;
fused non-aromatic heterocyclic groups such as dihydroindolyl (e.g., 2,3-dihydro-1H-indol-1-yl), dihydroisoindolyl (e.g., 1,3-dihydro-2H-isoindol-2-yl), dihydrobenzofuranyl (e.g., 2,3-dihydrobenzofuran-5-yl), dihydrobenzodioxinyl (e.g., 2,3-dihydro-1,4-benzodioxinyl), dihydrobenzodioxepinyl (e.g., 3,4-dihydro-2H-1,5-benzodioxepinyl), tetrahydrobenzofuranyl (e.g., 4,5,6,7-tetrahydrobenzofuran-3-yl), chromenyl (e.g., 4H-chromen-2-yl, 2H-chromen-3-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquiholinyl (e.g., 1,2,3,4-tetrahydroisoquinolin-4-yl), dihydrophthalazinyl (e.g., 1,4-dihydrophthalazin-4-yl) and the like;
   and the like can be mentioned.

The “cyclic group” optionally has substituent(s) (preferably 1 to 3 substituents) at substitutable position(s). When the number of the substituents is not less than 2, respective substituents may be the same or different.

As such substituents, for example,
(1) those exemplified as the substituents of the aforementioned “C₁₋₁₀ alkyl group” of the “C₁₋₁₀ alkyl group optionally having substituent(s)”;
(2) a C₁₋₆ alkyl group optionally substituted by 1 to 3 substituents selected from

(i) a halogen atom,

(ii) a carboxyl group,

(iii) a hydroxy group,

(iv) a C₁₋₆ alkoxy group,

(v) a C₁₋₆ alkoxy-carbonyl group,

(vi) a C₁₋₆ alkyl-carbonyloxy group (e.g., acetyloxy, tert-butylcarbonyloxy),

(vii) an amino group,

(viii) a carbamoyl group optionally mono- or di-substituted by a C₁₋₆ alkyl group optionally substituted by a hydroxy group,

(ix) a non-aromatic heterocyclic group (the non-aromatic heterocyclic group may be oxidized; e.g., piperidino, tetrahydrofuryl) optionally substituted by a C₁₋₆ alkyl group,

(x) a non-aromatic heterocyclylcarbonyl group (the non-aromatic heterocycle may be oxidized; e.g., morpholinylcarbonyl),

(xi) a C₆₋₁₄ aryl group (e.g., phenyl) optionally substituted by a C₁₋₆ alkylsulfonyl group,

(xii) a C₃₋₁₀ cycloalkyl group (e.g., cyclopropyl), and

(xiii) an aromatic heterocyclic group (e.g., furyl) optionally substituted by 1 to 3 substituents selected from a carboxyl group and a C₁₋₆ alkoxy-carbonyl group;
(3) a C₂₋₆ alkenyl group (e.g., ethenyl, 1-propenyl) optionally substituted by 1 to 3 substituents selected from

(i) a halogen atom,

(ii) a carboxyl group,

(iii) a C₁₋₆ alkoxy-carbonyl group,

(iv) a carbamoyl group, and

(v) a C₆₋₁₄ aryl group (e.g., phenyl) optionally substituted by a C₁₋₆ alkoxy-carbonyl group;
(4) a C₇₋₁₃ aralkyl group (e.g., benzyl) optionally substituted by 1 to 3 substituents selected from

(i) a C₁₋₆ alkyl group optionally substituted by 1 to 3 is halogen atoms,

(ii) a hydroxy group,

(iii) a C₁₋₆ alkoxy group, and

(iv) a halogen atom;

and the like can be mentioned.

Ra and Rb are each independently preferably a cyclic group optionally having substituent(s), or a C₁₋₁₀ alkyl group (preferably a C₁₋₆ alkyl group) optionally having substituent(s), more preferably a cyclic group optionally having substituent(s), further more preferably a C₆₋₁₄ aryl group (e.g., phenyl) optionally having substituent(s), a 5- or 6-membered aromatic heterocyclic group (e.g., pyridyl, thienyl, thiazolyl) optionally having substituent(s), a 5- or 6-membered non-aromatic heterocyclic group (e.g., pyrrolidinyl, piperidinyl, hexamethyleneiminyl, tetrahydrofuryl, tetrahydropyranyl, preferably a 5 or 6-membered non-aromatic nitrogen-containing heterocyclic group) optionally having substituent(s), or a C₃₋₁₀ cycloalkyl group optionally condensed with a benzene ring (e.g., cyclopropyl, cyclohexyl, indanyl, tetrahydronaphthyl), which optionally has substituent(s), still more preferably a C₆₋₁₄ aryl group (e.g., phenyl) optionally having substituent(s), a 5- or 6-membered non-aromatic heterocyclic group (e.g., pyrrolidinyl, piperidinyl, hexamethyleneiminyl, tetrahydrofuryl, tetrahydropyranyl, preferably a 5 or 6-membered non-aromatic nitrogen-containing heterocyclic group) optionally having substituent(s), or a C₃₋₁₀ cycloalkyl group condensed with a benzene ring (e.g., indanyl, tetrahydronaphthyl), which optionally has substituent(s), particularly preferably a phenyl group optionally having substituent(s), an indanyl group optionally having substituent(s) or a piperidinyl group optionally having substituent(s).

Ra is particularly preferably a phenyl group optionally having substituent(s), an indanyl group optionally having substituent(s) or a piperidinyl group optionally having substituent(s).

Rb is particularly preferably a phenyl group optionally having substituent(s).

As preferable substituents of the “cyclic group optionally having substituent(s)”, the “C₁₋₁₀ alkyl group optionally having substituent(s)” and the like for Ra or Rb, the following substituents can be mentioned.
(1) a halogen atom;
(2) a C₁₋₆ alkoxy group optionally substituted by 1 to 3 substituents selected from

(i) a carboxyl group,

(ii) a hydroxy group,

(iii) a C₁₋₆ alkoxy group,

(iv) a C₆₋₁₄ aryl group (e.g., pilenyl),

(v) a C₁₋₆ alkoxy-carbonyl group,

(vi) a C₁₋₆ alkylsulfonyl group,

(vii) a carbamoyl group, and

(viii) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group, and

(v) a non-aromatic heterocyclylcarbonyl group (the non-aromatic heterocycle may be oxidized; e.g., morpholinylcarbonyl);
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl), and
(c) an amino group optionally mono- or di-substituted by a C₁₋₆ alkyl-carbonyl group, and

(iii) a non-aromatic heterocyclic group (the non-aromatic heterocyclic group may be oxidized; e.g., tetrahydrofuryl) optionally substituted by a hydroxy group;
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom, a hydroxy group and a carbamoyl group;
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl) optionally substituted by 1 to 3 substituents selected from

(i) a C₁₋₆ alkyl group optionally substituted by a hydroxy group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group, and

(vi) a carbamoyl group optionally mono- or di-substituted by a C₁₋₆ alkyl group optionally substituted by 1 to 3 substituents selected from a hydroxy group and a carbamoyl group;
(11) a non-aromatic heterocyclyloxy group (the non-aromatic heterocycle may be oxidized; e.g., 1,1-dioxidotetrahydrothiopyranyloxy);
(12) a C₁₋₆ alkoxy-carbonyl group;
(13) a carboxyl group;
(14) a non-aromatic heterocyclylcarbonyl group (the non-aromatic heterocycle may be oxidized; e.g., morpholinylcarbonyl);
(15) a C₁₋₄ alkylenedioxy group optionally substituted by a halogen atom;
(16) a C₆₋₁₄ aryl group (e.g., phenyl) optionally substituted by a C₁₋₆ alkoxy group;
(17) an aromatic heterocyclic group (e.g., thienyl, tetrazolyl);
   and the like.

As other preferable substituents, the following substituents can be mentioned.
(1) a halogen atom;
(2) a C₁₋₆ alkoxy group optionally substituted by 1 to 3 substituents selected from

(i) a carboxyl group,

(ii) a hydroxy group,

(iii) a C₁₋₆ alkoxy group,

(iv) a C₆₋₁₄ aryl group (e.g., phenyl),

(v) a C₁₋₆ alkoxy-carbonyl group,

(vi) a C₁₋₆ alkylsulfonyl group,

(vii) a carbamoyl group, and

(viii) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group,

(v) a non-aromatic heterocyclylcarbonyl group (the non-aromatic heterocycle may be oxidized; e.g., morpholinylcarbonyl),

(vi) a C₆₋₁₄ aryl group (e.g., phenyl) optionally substituted by a C₁₋₆ alkylsulfonyl group,

(vii) a C₃₋₁₀ cycloalkyl group (e.g., cyclopropyl),

(viii) an aromatic heterocyclic group (e.g., furyl) optionally substituted by 1 to 3 substituents selected from a carboxyl group and a C₁₋₆ alkoxy-carbonyl group,

(ix) a non-aromatic heterocyclic group (the non-aromatic heterocyclic group may be oxidized; e.g., tetrahydrofuryl) optionally substituted by a C₁₋₆ alkyl group, and

(x) a C₁₋₆ alkoxy group;
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl),
(c) an amino group optionally mono- or di-substituted by a C₁₋₆ alkyl-carbonyl group,
(d) a alkoxy group optionally substituted by a C₁₋₆ alkoxy group, and
(e) an aromatic heterocyclic group (e.g., thienyl),

(iii) a non-aromatic heterocyclic group (the non-aromatic heterocyclic group may be oxidized; e.g., tetrahydrofuryl, tetrahydropyranyl) optionally substituted by a hydroxy group, and

(iv) a C₁₋₆ alkoxy-carbonyl group optionally substituted by a C₆₋₁₄ aryl group (e.g., phenyl);
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom, a hydroxy group, a carbamoyl group and an aromatic heterocyclic group (e.g., furyl);
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl, tetrahydropyranyl) optionally substituted by 1 to 3 substituents selected from

(i) a C₁₋₆ alkyl group optionally substituted by 1 to 3 substituents selected from
(a) a hydroxy group,
(b) a C₆₋₁₄ aryl group (e.g., phenyl),
(c) a C₁₋₆ alkoxy group, and
(d) a non-aromatic heterocyclic group (the non-aromatic heterocyclic group may be oxidized; e.g., tetrahydrofuryl) optionally substituted by a C₁₋₆ alkyl group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group,

(vi) a carbamoyl group optionally mono- or di-substituted by a C₁₋₆ alkyl group optionally substituted by 1 to 3 substituents selected from a hydroxy group and a carbamoyl group,

(vii) a hydroxy group,

(viii) a C₆₋₁₄ aryl-carbonyl group (e.g., benzoyl),

(ix) a C₁₋₆ alkylsulfonyl group, and

(x) a C₆₋₁₄ arylsulfonyl group (e.g., phenylsulfonyl);
(11) a non-aromatic heterocyclyloxy group (the non-aromatic heterocycle may be oxidized; e.g., 1,1-dioxidotetrahydrothiopyranyloxy);
(12) a C₁₋₆ alkoxy-carbonyl group optionally substituted by a C₆₋₁₄ aryl group (e.g., phenyl);
(13) a carboxyl group;
(14) a non-aromatic heterocyclylcarbonyl group (the non-aromatic heterocycle may be oxidized; e.g., morpholinylcarbonyl, piperazinylcarbonyl) optionally, substituted by a C₁₋₆ alkyl group optionally substituted by a C₆₋₁₄ aryl group (e.g., phenyl);
(15) a C₁₋₄ alkylenedioxy group optionally substituted by a halogen atom;
(16) a C₆₋₁₄ aryl group (e.g., phenyl) optionally substituted by 1 to 3 substituents selected from a C₁₋₆ alkoxy group and a C₁₋₆ alkylsulfonyl group;
(17) an aromatic heterocyclic group (e.g., thienyl, pyridyl, tetrazolyl);
(18) a C₁₋₆ alkyl-carbonyl group optionally substituted by a hydroxy group;
(19) a C₆₋₁₄ aryl-carbonyl group (e.g., benzoyl);
(20) an oxo group;
(21) a C₁₋₆ alkylsulfonyl group optionally substituted by 1 to 3 halogen atoms;
(22) a C₆₋₁₄ arylsulfonyl group (e.g., phenylsulfonyl) optionally substituted by a C₁₋₆ alkoxy group;
(23) a C₃₋₁₀ cycloalkylsulfonyl group (e.g., cyclopropylsulfonyl);
(24) an aromatic heterocyclylsulfonyl group (e.g., pyridylsulfonyl, pyrazolylsulfonyl, thienylsulfonyl, furylsulfonyl, imidazolylsulfonyl) optionally substituted by 1 to 3 substituents selected from

(i) a C₁₋₆ alkyl group,

(ii) a C₁₋₆ alkoxy group,

(iii) a C₁₋₆ alkoxy-carbonyl group, and

(iv) a halogen atom;
(25) a C₁₋₆ alkylthio group (e.g., methylthio);
   and the like.

Preferable embodiment of Ra is
(A) a C₆₋₁₄ aryl group (e.g., phenyl) optionally substituted by 1 to 3 substituents selected from
(1) a halogen atom;
(2) a C₁₋₆ alkoxy group optionally substituted by 1 to 3 substituents selected from

(i) a hydroxy group,

(ii) a C₁₋₆ alkoxy group,

(iii) a C₆₋₁₄ aryl group (e.g., phenyl),

(iv) a C₁₋₆ alkylsulfonyl group, and

(v) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by. 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group, and

(v) a non-aromatic heterocyclylcarbonyl group (the non-aromatic heterocycle may be oxidized; e.g., morpholinylcarbonyl);
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl), and
(c) an amino group optionally mono- or di-substituted by a C₁₋₆ alkyl-carbonyl group, and

(iii) a non-aromatic heterocyclic group (the non-aromatic heterocyclic group may be oxidized; e.g., tetrahydrofuryl) optionally substituted by a hydroxy group;
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom and a hydroxy group;
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl) optionally substituted by 1 to 3 substituents selected from

(i) a C₁₋₆ alkyl group optionally substituted by a hydroxy group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group, and

(vi) a carbamoyl group optionally mono- or di-substituted by a C₁₋₆ alkyl group optionally substituted by 1 to 3 substituents selected from a hydroxy group and a carbamoyl group;
(11) a non-aromatic heterocyclyloxy group (the non-aromatic heterocycle may be oxidized; e.g., 1,1-dioxidotetrahydrothiopyranyloxy);
(12) a C₁₋₆ alkoxy-carbonyl group;
(13) a carboxyl group;
(14) a non-aromatic heterocyclylcarbonyl group (the non-aromatic heterocycle may be oxidized; e.g., morpholinylcarbonyl);
(15) a C₁₋₄ alkylenedioxy group optionally substituted by a halogen atom; and
(16) an aromatic heterocyclic group (e.g., tetrazolyl);
(B) a 5 or 6-membered aromatic heterocyclic group (e.g., pyridyl, thienyl);
(C) C₃₋₁₀ cycloalkyl group condensed with a benzene ring (e.g., indanyl, tetrahydronaphthyl); or
(D) a C₁₋₆ alkyl group optionally substituted by 1 to 3 substituents selected from
(1) a carbamoyl group optionally mono- or di-substituted by a C₁₋₆ alkyl group optionally substituted by 1 to 3 substituents selected from a hydroxy group and a carbamoyl group;
(2) a C₆₋₁₄ aryl group (e.g., phenyl) optionally substituted by a C₁₋₆ alkoxy group; and
(3) an aromatic heterocyclic group (e.g., thienyl).

Another preferable embodiment of Ra is
(A) a C₆₋₁₄ aryl group (e.g., phenyl) optionally substituted by 1 to 3 substituents selected from
(1) a halogen atom;
(2) a C₁₋₆ alkoxy group optionally substituted by 1 to 3 substituents selected from

(i) a hydroxy group,

(ii) a C₁₋₆ alkoxy group,

(iii) a C₆₋₁₄ aryl group (e.g., phenyl),

(iv) a C₁₋₆ alkylsulfonyl group, and

(v) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group, and

(v) a non-aromatic heterocyclylcarbonyl group (the non-aromatic heterocycle may be oxidized; e.g., morpholinylcarbonyl);
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),
(ii) a C₁₋₆ alkyl-carbonyl group optionally substituted by 1 to 3 substituents selected from
   (a) a carboxyl group,
   (b) a C₆₋₁₄ aryl group (e.g., phenyl), and
   (c) an amino group optionally mono- or di-substituted by a C₁₋₆ alkyl-carbonyl group, and
(iii) a non-aromatic heterocyclic group (the non-aromatic heterocyclic group may be oxidized; e.g., tetrahydrofuryl) optionally substituted by a hydroxy group;
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom and a hydroxy group;
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl) optionally substituted by 1 to 3 substituents selected from

(i) a C₁₋₆ alkyl group optionally substituted by a hydroxy group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group, and

(vi) a carbamoyl group optionally mono- or di-substituted by a C₁₋₆ alkyl group optionally substituted by 1 to 3 substituents selected from a hydroxy group and a carbamoyl group;
(11) a non-aromatic heterocyclyloxy group (the non-aromatic heterocycle may be oxidized; e.g., 1,1-dioxidotetrahydrothiopyranyloxy);
(12) a C₁₋₆ alkoxy-carbonyl group;
(13) a carboxyl group;
(14) a non-aromatic heterocyclylcarbonyl group (the non-aromatic heterocycle may be oxidized; e.g., morpholinylcarbonyl);
(15) a C₁₋₄ alkylenedioxy group optionally substituted by a halogen atom;
(16) an aromatic heterocyclic group (e.g., tetrazolyl); and
(17) a C₁₋₆ alkylsulfonyl group;
(B) a 5 or 6-membered aromatic heterocyclic group (e.g., pyridyl, thienyl);
(C) a 5 or 6-membered non-aromatic heterocyclic group (the non-aromatic heterocyclic group may be oxidized; e.g., pyrrolidinyl, piperidinyl, hexamethyleneiminyl, tetrahydrofuryl, tetrahydropyranyl) optionally substituted by 1 to 3 substituents selected from
(1) a C₁₋₆ alkyl group optionally substituted by 1 to 3 substituents selected from

(i) a C₆₋₁₄ aryl group (e.g., phenyl) optionally substituted by a C₁₋₆ alkylsulfonyl group,

(ii) a C₃₋₁₀ cycloalkyl group (e.g., cyclopropyl),

(iii) an aromatic heterocyclic group (e.g., furyl) optionally substituted by 1 to 3 substituents selected from a carboxyl group and a C₁₋₆ alkoxy-carbonyl group,

(iv) a non-aromatic heterocyclic group (the non-aromatic heterocyclic group may be oxidized; e.g., tetrahydrofuryl) optionally substituted by a C₁₋₆ alkyl group, and

(v) a C₁₋₆ alkoxy group;
(2) a C₁₋₆ alkyl-carbonyl group optionally substituted by a hydroxy group;
(3) a C₁₋₆ alkoxy-carbonyl group optionally substituted by a C₆₋₁₄ aryl group (e.g., phenyl);
(4) a C₆₋₁₄ aryl-carbonyl group (e.g., benzoyl);
(5) an oxo group;
(6) a hydroxy group;
(7) a C₁₋₆ alkylsulfonyl group optionally substituted by 1 to 3 halogen atoms;
(8) a C₆₋₁₄ arylsulfonyl group (e.g., phenylsulfonyl) optionally substituted by a C₁₋₆ alkoxy group;
(9) a C₃₋₁₀ cycloalkylsulfonyl group (e.g., cyclopropylsulfonyl);
(10) an aromatic heterocyclylsulfonyl group (e.g., pyridylsulfonyl, pyrazolylsulfonyl, thienylsulfonyl, furylsulfonyl, imidazolylsulfonyl) optionally substituted by 1 to 3 substituents selected from

(i) a C₁₋₆ alkyl group,

(ii) a C₁₋₆ alkoxy group,

(iii) a C₁₋₆ alkoxy-carbonyl group, and

(iv) a halogen atom;
(11) a C₆₋₁₄ aryl group (e.g., phenyl) optionally substituted by a C₁₋₆ alkylsulfonyl group; and
(12) an aromatic heterocyclic group (e.g., pyridyl, thienyl);
(D) a C₃₋₁₀ cycloalkyl group condensed with a benzene ring (e.g., indanyl, tetrahydronaphthyl); or
(E) a C₁₋₆ alkyl group optionally substituted by 1 to 3 substituents selected from
(1) a hydroxy group;
(2) a C₁₋₆ alkoxy group;
(3) a C₁₋₆ alkylthio group (e.g., methylthio);
(4) a C₆₋₁₄ aryl group (e.g., phenyl) optionally substituted by a C₁₋₆ alkoxy group;
(5) an aromatic heterocyclic group (e.g., thienyl, pyridyl);
(6) a non-aromatic heterocyclic group (the non-aromatic heterocyclic group may be oxidized; e.g., piperidinyl, tetrahydropyranyl) optionally substituted by 1 to 3 substituents selected from

(i) a hydroxy group,

(ii) a C₁₋₆ alkyl group optionally substituted by 1 to 3 substituents selected from
(a) a C₆₋₁₄ aryl group (e.g., phenyl),
(b) a C₃₋₆ alkoxy group, and
(c) a non-aromatic heterocyclic group (the non-aromatic heterocyclic group may be oxidized; e.g., tetrahydrofuryl) optionally substituted by a C₁₋₆ alkyl group,

(iii) a C₁₋₆ alkyl-carbonyl group,

(iv) a C₆₋₁₄ aryl-carbonyl group (e.g., benzoyl),

(v) a C₁₋₆ alkylsulfonyl group, and

(vi) a C₆₋₁₄ arylsulfonyl group (e.g., phenylsulfonyl);
(7) a non-aromatic heterocyclylcarbonyl group (the non-aromatic heterocycle may be oxidized; e.g., morpholinylcarbonyl, piperazinylcarbonyl) optionally substituted by C₁₋₆ alkyl group optionally substituted by a C₆₋₁₄ aryl group (e.g., phenyl);
(8) an amino group optionally mono- or di-substituted by substituent(s) selected from

(i) a C₁₋₆ alkoxy-carbonyl group optionally substituted by a C₆₋₁₄ aryl group (e.g., phenyl),

(ii) a non-aromatic heterocyclic group (the non-aromatic heterocyclic group may be oxidized; e.g., tetrahydropyranyl), and

(iii) a C₁₋₆ alkyl-carbonyl group optionally substituted by 1 to 3 substituents selected from
(a) an amino group optionally mono- or di-substituted by a C₁₋₆ alkyl-carbonyl group,
(b) a C₁₋₆ alkoxy group optionally substituted by a C₁₋₆ alkoxy group, and
(c) an aromatic heterocyclic group (e.g., thienyl); and
(9) a carbamoyl group optionally mono- or di-substituted by a C₁₋₆ alkyl group optionally substituted by 1 to 3 substituents selected from a hydroxy group, a carbamoyl group and an aromatic heterocyclic group (e.g., furyl).

Preferable embodiment of Rb is
(A) a C₆₋₁₄ aryl group (e.g., phenyl) optionally substituted by 1 to 3 substituents selected from
(1) a halogen atom;
(2), a hydroxy group; and
(3) a C₁₋₆ alkoxy group optionally substituted by 1 to 3 substituents selected from

(i) a C₆₋₁₄ aryl group (e.g., phenyl),

(ii) a carboxyl group,

(iii) a C₁₋₆ alkoxy-carbonyl group, and

(iv) a carbamoyl group;
(B) a 5 or 6-membered aromatic heterocyclic group (e.g., pyridyl, thiazolyl, thienyl);
(C) a C₁₋₆ alkyl group (e.g., methyl, propyl); or
(D) a C₃₋₁₀ cycloalkyl group (e.g., cyclopropyl, cyclohexyl).

Another preferable embodiment of Rb is
(A) a C₆₋₁₄ aryl group (e.g., phenyl) optionally substituted by 1 to 3 substituents selected from
(1) a halogen atom;
(2) a hydroxy group; and
(3) a C₁₋₆ alkoxy group optionally substituted by 1 to 3 substituents selected from

(i) a C₆₋₁₄ aryl group (e.g., phenyl),

(ii) a carboxyl group,

(iii) a C₁₋₆ alkoxy-carbonyl group,

(iv) a carbamoyl group, and

(v) a C₁₋₆ alkoxy group;
(B) a 5 or 6-membered aromatic heterocyclic group (e.g., pyridyl, thiazolyl, thienyl);
(C) a C₁₋₆ alkyl group (e.g., methyl, propyl); or
(D) a C₃₋₁₀ cycloalkyl group (e.g., cyclopropyl, cyclohexyl).

Ring A is a 5- or 6-membered aromatic heterocycle optionally having substituent(s).

As the “5- or 6-membered aromatic heterocycle” of the “5- or 6-membered aromatic heterocycle optionally having substituent(s)” for ring A, for example, a 5- or 6-membered ring, from among the rings constituting the aromatic heterocyclic groups exemplified as the “cyclic group” of the “cyclic group optionally having substituent(s)” for Ra or Rb, can be mentioned. As preferable examples of the 5- or 6-membered aromatic heterocycle, furan, thiophene, pyridine, pyrimidine, pyridazine, pyrazine, triazine (1,3,5-triazine, 1,2,3-triazine, 1,3,4-triazine), pyrrole, imidazole, pyrazole, thiazole, isothizole, oxazole, isoxazole, oxadiazole (1,2,4-oxadiazole, 1,3,4-oxadiazole), thiadiazole (1,2,4-thiadiazole, 1,3,4-thiadiazole), triazole (1,2,3-triazole, 1,2,4-triazole), tetrazole and the like can be mentioned.

The “5- or 6-membered aromatic heterocycle” of the “5- or 6-membered aromatic heterocycle optionally having substituent(s)” for ring A is preferably pyrrole, pyrazole, triazole (e.g., 1,2,3-triazole, 1,2,4-triazole), imidazole thiophene or pyridine, more preferably a 5-membered aromatic heterocycle, further more preferably pyrrole, pyrazole, triazole (e.g., 1,2,3-triazole, 1,2,4-triazole), imidazole or thiophene, still more preferably pyrrole, pyrazole, 1,2,3-triazole or imidazole, particularly preferably imidazole or pyrrole, most preferably imidazole.

The “5- or 6-membered aromatic heterocycle” optionally has substituent(s) (preferably 1 to 3 substituents) at substitutable position(s). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

As preferable substituents of ring A,
(1) a C₁₋₆ alkyl group optionally substituted by a C₁₋₆ alkoxy group;
(2) a C₆₋₁₄ aryl group (e.g., phenyl);
(3) a C₁₋₆ alkyl-carbonyl group;
   and the like can be mentioned.

As another preferable substituents of ring A,
(1) a halogen atom;
(2) a C₁₋₆ alkyl group optionally substituted by 1 to 3 substituents selected from

(a) a hydroxy group,

(b) a C₁₋₆ alkoxy group,

(c) an amino group,

(d) a carbamoyl group optionally mono- or di-substituted by a C₁₋₆ alkyl group optionally substituted by a hydroxy group,

(e) a C₆₋₁₄ aryl group (e.g., phenyl) optionally substituted by an aromatic heterocyclic group (e.g., pyrrolyl),

(f) an aromatic heterocyclic group (e.g., thiazolyl), and

(g) a non-aromatic heterocyclic group (e.g., morpholinyl);
(3) a C₆₋₁₄ aryl group (e.g., phenyl);
(4) a C₁₋₆ alkyl-carbonyl group;
(5) a alkoxy group;
(6) a formyl group;
   and the like can be mentioned.

Preferable embodiment of ring A is a 5-membered aromatic heterocycle (preferably pyrrole, pyrazole, 1,2,3-triazole, imidazole or thiophene, more preferably pyrrole, pyrazole, 1,2,3-triazole or imidazole, particularly preferably imidazole or pyrrole, most preferably imidazole) optionally substituted by 1 to 3 substituents selected from
(1) a C₁₋₆ alkyl group optionally substituted by a C₁₋₆ alkoxy group;
(2) a C₆₋₁₄ aryl group (e.g., phenyl); and
(3) a C₁₋₆ alkyl-carbonyl group.

Another preferable embodiment of ring A is a 5 or 6-membered aromatic heterocycle (preferably pyrrole, pyrazole, triazole (1,2,3-triazole, 1,2,4-triazole), imidazole, thiophene or pyridine, more preferably a 5-membered aromatic heterocycle, further more preferably pyrrole, pyrazole, triazole (1,2,3-triazole, 1,2,4-triazole), imidazole or thiophene, still more preferably pyrrole, pyrazole, 1,2,3-triazole or imidazole, particularly preferably imidazole or pyrrole, most preferably imidazole) optionally substituted by 1 to 3 substituents selected from
(1) a halogen atom;
(2) a C₁₋₆ alkyl group optionally substituted by 1 to 3 substituents selected from

(a) a hydroxy group,

(b) a C₁₋₆ alkoxy group,

(c) an amino group,

(d) a carbamoyl group optionally mono- or di-substituted by a C₁₋₆ alkyl group optionally substituted by a hydroxy group,

(e) a C₆₋₁₄ aryl group (e.g., phenyl) optionally substituted by an aromatic heterocyclic group (e.g., pyrrolyl),

(f) an aromatic heterocyclic group (e.g., thiazolyl), and

(g) a non-aromatic heterocyclic group (e.g., morpholinyl);
(3) a C₆₋₁₄ aryl group (e.g., phenyl);
(4) a C₁₋₆ alkyl-carbonyl group;
(5) a C₁₋₆ alkoxy group; and
(6) a formyl group.

Rc is a hydrocarbon group optionally containing heteroatom(s) as the constituting atom(s), which optionally has substituent(s).

As the “hydrocarbon group” of the “hydrocarbon group optionally containing heteroatom(s) as the constituting atom(s), which optionally has substituent(s)” for Rc, for example, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₃ aralkyl group, a C₈₋₁₃ arylalkenyl group, a C₃₋₁₀ cycloalkyl-C₁₋₆ alkyl group and the like can be mentioned.

As used herein, as the C₁₋₁₀ alkyl group, for example, those similar to the “C₁₋₁₀ alkyl group” of the “C₁₋₁₀ alkyl group optionally having substituent(s)” for Ra or Rb can be mentioned.

As the C₂₋₁₀ alkenyl group, for example, those similar to the “C₂₋₁₀ alkenyl group” of the “C₂₋₁₀ alkenyl group optionally having substituent(s)” for Ra or Rb can be mentioned.

As the C₂₋₁₀ alkynyl group, for example, those similar to the “C₂₋₁₀ alkynyl group” of the “C₂₋₁₀ alkynyl group optionally having substituent(s)” for Ra or Rb can be mentioned.

As the C₃₋₁₀ cycloalkyl group, for example, those exemplified as the “cyclic group” of the “cyclic group optionally having substituent(s)” for Ra or Rb can be mentioned.

As the C₃₋₁₀ cycloalkenyl group, for example, those exemplified as the “cyclic group” of the “cyclic group optionally having substituent(s)” for Ra or Rb can be mentioned.

As the C₄₋₁₀ cycloalkadienyl group, for example, those exemplified as the “cyclic group” of the “cyclic group optionally having substituent(s)” for Ra or Rb can be mentioned.

The aforementioned C₃₋₁₀ cycloalkyl group, C₃₋₁₀ cycloalkenyl group and C₄₋₁₀ cycloalkadienyl group are each optionally condensed with a benzene ring, and as such a fused ring group, for example, indanyl, dihydronaphthyl, tetrahydronaphthyl, fluorenyl and the like can be mentioned.

As the C₆₋₁₄ aryl group, for example, those exemplified as the “cyclic group” of the “cyclic group optionally having substituent(s)” for Ra or Rb can be mentioned.

As the C₇₋₁₃ aralkyl group, for example, benzyl, phenethyl, naphthylmethyl, biphenylylmethyl and the like can be mentioned.

As the C₈₋₁₃ arylalkenyl group, for example, styryl and the like can be mentioned.

As the C₃₋₁₀ cycloalkyl-C₁₋₆ alkyl group, for example, cyclohexylmethyl and the like can be mentioned.

The aforementioned C₁₋₁₀ alkyl group, C₂₋₁₀ alkenyl group and C₂₋₁₀ alkynyl group, which are exemplified as the “hydrocarbon group”, optionally have substituent(s) (preferably 1 to 3 substituents) at substitutable position(s). As such substituents, for example, those similar to the substituents which the “C₁₋₁₀ alkyl group” of the “C₁₋₁₀ alkyl group optionally having substituent(s)” for Ra or Rb optionally has, can be mentioned. When the number of the substituents is not less than 2, respective substituents may be the same or different.

The aforementioned C₃₋₁₀ cycloalkyl group, C₃₋₁₀ cycloalkenyl group, C₄₋₁₀ cycloalkadienyl group, C₆₋₁₄ aryl group, C₇₋₁₃ aralkyl group, C₈₋₁₃ arylalkenyl group and C₃₋₁₀ cycloalkyl-C₁₋₆ alkyl group, which are exemplified as the “hydrocarbon group”, optionally have substituent(s) (preferably 1 to 3 substituents) at substitutable position(s). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

The “hydrocarbon group optionally containing heteroatom(s) as the constituting atom(s)” of the “hydrocarbon group optionally containing heteroatom(s) as the constituting atom(s), which optionally has substituent(s)” for Rc means, for example, when the “hydrocarbon group” is a chain hydrocarbon group (a alkyl group, a C₂₋₁₀ alkenyl group or a C₂₋₁₀ alkynyl group), a group in which the carbon atom(s) in the main chain of the chain hydrocarbon group is (are) replaced by heteroatom(s) selected from O, N and S. As preferable examples thereof, the following groups can be mentioned:

D¹-O-D²-

D¹-NH-D²-

D¹-S-D²-

wherein
D¹ is a hydrogen atom or a C₁₋₉ chain hydrocarbon group, D² is a bond or a divalent C₁₋₉ chain hydrocarbon group, provided that when both D¹ and D² are C₁₋₉ chain hydrocarbon groups, then the total of the carbon number of the C₁₋₉ chain hydrocarbon group for D¹ and the carbon number of the C₁₋₉ chain hydrocarbon group for D² should be not more than 9. S may be oxidized.

When the “hydrocarbon group” is a cyclic hydrocarbon group (a C₃₋₁₀ cycloalkyl group, a C₃₋₁₀ cycloalkenyl group, a C₄₋₁₀ cycloalkadienyl group or a C₆₋₁₄ aryl group), a group in which the carbon atom(s) among ring-constituting atoms of the cyclic hydrocarbon group is (are) replaced by heteroatom(s) selected from O, N and S. When the group contains S, S may be oxidized. As preferable examples thereof, those similar to the aromatic heterocyclic group and the non-aromatic heterocyclic group exemplified as the “cyclic group” of the “cyclic group optionally having substituent(s)” for Ra or Rb can be mentioned.

When the “hydrocarbon group” is a cyclic hydrocarbon-chain hydrocarbon group (a C₇₋₁₃ aralkyl group, a C₈₋₁₃ arylalkenyl group or a C₃₋₁₀ cycloalkyl-C₁₋₆ alkyl group), as the chain hydrocarbon group and the cyclic hydrocarbon group, those similar to the aforementioned groups.

The “hydrocarbon group optionally containing heteroatom(s) as the constituting atom(s)” optionally has substituent(s) (preferably 1 to 3 substituents) at substitutable position(s). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

As preferable examples of Rc, the following groups can be mentioned:

### 1) Type 1 a Group Represented by the Formula:

R³—(Z₁)q-(Z)p-

wherein
R³ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₁ is —CO—, —O—, —S—, —S(O)— or —S(O)₂—; and
p and q are each independently 0 or 1;

### 2) Type 2 a Group Represented by the Formula:

R⁴—Z₂—(R⁵)C(R⁶)—(Z)p-

wherein
R⁴ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
R⁵ and R⁶ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s), or R⁵ and R⁶ in combination form an oxo group;
Z is a C₁₋₄ alkylene group;
Z₂ is —O—, or a group represented by the formula: —N(R⁷)— (wherein R⁷ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s));
p is 0 or 1; and
when Z₂ is a group represented by the formula: —N(R⁷)—, then R⁴ and R⁷ are optionally bonded to each other to form, together with the adjacent nitrogen atom, a nitrogen-containing heterocycle optionally having substituent(s);

### 3) Type 3 a Group Represented by the Formula:

R⁹—Z₃—V(R⁹)—(Z)p-

wherein
R⁸ and R⁹ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₃ is —CO—, —CONH— or —SO₂—; and
p is 0 or 1;

### 4) Type 4 a Group Represented by the Formula:

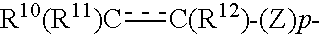

wherein
R¹⁰, R¹¹ and R¹² are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
[symbol] is a single bond or a double bond; and
p is 0 or 1; and

### 5) Type 5 a Group Represented by the Formula:

R¹³O—N═C(R¹⁴)—(Z)p-

wherein
R¹³ and R¹⁴ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group; and
p is 0 or 1.

In the formula of Type 5, the configuration of R¹³O may be either at the cis-position (Z isomer) or at the trans-position (E isomer) relative to R¹⁴, or may be a mixture thereof at any ratio.

In the above formulas, as the “cyclic group optionally having substituent(s)” for R³, R⁴, R⁵, R⁶, R⁷, R₈, R⁹, R¹⁰, R¹¹ or R¹², for example, those similar to the “cyclic group optionally having substituent(s)” for Ra or Rb can be mentioned.

In the above formulas, as the “C₁₋₁₀ alkyl group optionally having substituent(s)” for R³, R⁴, R⁵, R⁶, R⁷, R⁸, R⁹, R¹⁰, R¹¹ or R¹², for example, those similar to the “C₁₋₁₀ alkyl group optionally having substituent(s)” for Ra or Rb can be mentioned.

In the above formulas, as the “C₂₋₁₀ alkenyl group optionally having substituent(s)” for R³, R⁴, R⁵, R⁶, R⁷, R⁸, R⁹, R¹⁰, R¹¹ or R¹², for example, those similar to the “C₂₋₁₀ alkenyl group optionally having substituent(s)” for Ra or Rb can be mentioned.

In the above formulas, as the “C₂₋₁₀ alkynyl group optionally having substituent(s)” for R³, R⁴, R⁵, R⁶, R⁷, R⁸, R₉, R¹⁰, R¹¹ or R¹², for example, those similar to the “C₂₋₄₀ alkynyl group optionally having substituent(s)” for Ra or Rb can be mentioned.

In the above formulas, the “C₁₋₄ alkylene group” for Z may be straight or branched chain, and for example, —CH₂—, —CH₂CH₂—, —CH₂CH₂CH₂—, —CH(CH₃)CH₂—, —CH₂CH(CH₃) —, —C(CH₃)₂—, —CH(C₂H₅)—, —CH₂CH₂CH₂CH₂—, —CH(CH₃)CH₂CH₂—, —CH₂CH(CH₃)CH₂—, —CH₂CH₂CH(CH₃)—, —CH(C₃H₇)—, —CH(CH(CH₃)₂) —, —(CH(CH₃))₂— and the like can be mentioned.

As the “nitrogen-containing heterocycle” of the “nitrogen-containing heterocycle optionally having substituent(s)”, which is formed, together with the adjacent nitrogen atom, by R⁴ and R⁷ bonded to each other, for example, a 5- to 7-membered nitrogen-containing heterocycle containing, as a ring-constituting atom besides carbon atoms, at least one nitrogen atom and optionally further containing one to two heteroatoms selected from an oxygen atom, a sulfur atom and a nitrogen atom can be mentioned. As preferable examples of the “nitrogen-containing heterocycle”, pyrrolidine, imidazolidine, pyrazolidine, piperidine, piperazine, morpholine, thiomorpholine, oxopiperazine and the like can be mentioned.

The “nitrogen-containing heterocycle” optionally has substituent(s) (preferably 1 to 3 substituents, more preferably 1 or 2 substituents) at substitutable position(s). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

Preferable examples of each type are as follows:

### 1) Type 1 a Group Represented by the Formula:

R³—(Z₁)q-(Z)p-

wherein
R³ is
(1) a hydrogen atom,
(2) a cyclic group (preferably a C₆₋₁₄ aryl group (e.g., phenyl) or a 5- or 6-membered aromatic heterocyclic group (e.g., imidazolyl, thienyl)) optionally having substituent(s) (the substituent(s) is (are) 1 to 3 selected from

(i) a carboxyl group;

(ii) a C₁₋₆ alkoxy group optionally substituted by 1 to 3 substituents selected front
(a) a C₁₋₆ alkoxy group,
(b) a carbamoyl group optionally mono- or di-substituted by a C₁₋₆ alkyl group optionally substituted by a carbamoyl group, and
(c) a carboxyl group;

(iii) a hydroxy group;

(iv) a carbamoyl group optionally mono- or di-substituted by substituent(s) selected from
(a) a C₁₋₆ alkyl group optionally substituted by a hydroxy group, and
(b) a C₁₋₆ alkylsulfonyl group;

(v) a C₁₋₆ alkyl group optionally, substituted by a carboxyl group;

(vi) a C₁₋₄ alkylenedioxy group;

(vii) an amino group optionally mono- or di-substituted by a C₁₋₆ alkyl-carbonyl group;

(viii) a sulfamoyl group optionally mono- or di-substituted by a alkyl-carbonyl group;

(ix) an aromatic heterocyclic group (e.g., tetrazolyl); and

(x) a non-aromatic heterocyclic group (e.g., dihydroxadiazolyl) optionally substituted by 1 to 3 substituents selected from an oxo group and a thioxo group),
(3) a C₁₋₁₀ alkyl group (preferably a C₁₋₆ alkyl group) optionally having substituent(s) (the substituent(s) is (are) 1 to 3 selected from a hydroxy group, a carboxyl group and a carbamoyl group),
(4) a C₂₋₁₀ alkenyl group (preferably a C₂₋₆ alkenyl group) optionally having substituent(s) (the substituent(s) is (are) 1 to 3 selected from a hydroxy group, a carboxyl group and a carbamoyl group), or
(5) a C₂₋₁₀ alkynyl group (preferably a C₂₋₆ alkynyl group) optionally having substituent(s) (the substituent(s) is (are) 1 to 3 selected from a hydroxy group, a carboxyl group and a carbamoyl group);
Z is a C₁₋₄ alkylene group;
Z₁ is —CO—, —O—, —S—, —S(O)— or —S(O)₂—; and
p and q are each independently 0 or 1.

### 2) Type 2 a Group Represented by the Formula:

R⁴—Z₂—(R⁶)C(R⁶)—(Z)p-

wherein
R⁴ is
(1) a hydrogen atom,
(2) a cyclic group (preferably a C₆₋₁₄ aryl group (e.g., phenyl) or a 5- or 6-membered aromatic heterocyclic group (e.g., pyridyl)) optionally having substituent(s),
(3) a C₁₋₁₀ alkyl group (preferably a C₁₋₆ alkyl group) optionally having substituent(s) (the substituent(s) is (are) 1 to 3 selected from a carbamoyl group and a C₆₋₁₄ aryl group (e.g., phenyl)),
(4) a C₂₋₁₀ alkenyl group (preferably a C₂₋₆ alkenyl group) optionally having substituent(s) (the substituent(s) is (are) 1 to 3 selected from a carbamoyl group and a C₆₋₁₄ aryl group (e.g., phenyl)), or
(5) a C₂₋₁₀ alkynyl group (preferably a C₂₋₆ alkynyl group) optionally having substituent(s) (the substituent(s) is (are) 1 to 3 selected from a carbamoyl group and a C₆₋₁₄ aryl group (e.g., phenyl));
R⁵ and R⁶ are each independently
(1) a hydrogen atom,
(2) a cyclic group (preferably a C₃₋₁₀ cycloalkyl group (e.g., cyclopropyl) or a C₆₋₁₄ aryl group (e.g., phenyl)) optionally having substituent(s),
(3) a C₁₋₁₀ alkyl group (preferably a C₁₋₆ alkyl group) optionally having substituent(s) (the substituent(s) is (are) 1 to 3 selected from

(i) a C₁₋₆ alkoxy group;

(ii) an amino group optionally mono- or di-substituted by substituent(s) selected from a C₁₋₆ alkyl group and a C₁₋₆ alkyl-carbonyl group;

(iii) a carboxyl group; and

(iv) a C₁₋₆ alkoxy-carbonyl group),
(4) a C₂₋₁₀ alkenyl group (preferably a C₂₋₆ alkenyl group) optionally having substituent(s), or
(5) a C₂₋₁₀ alkynyl group (preferably a C₂₋₆ alkynyl group) optionally having substituent(s), or
R⁵ and R⁶ in combination form an oxo group;
Z is a C₁₋₄ alkylene group;
Z₂ is —O—, or a group represented by the formula: —N(R⁷)— [wherein R⁷ is a hydrogen atom, a cyclic group (preferably a C₃₋₁₀ cycloalkyl group (e.g., cyclopropyl), a C₆₋₁₄ aryl group (e.g., phenyl) or a 5- or 6-membered aromatic heterocyclic group (e.g., pyridyl)) optionally having substituent(s), a C₁₋₁₀ alkyl group (preferably a C₁₋₆ alkyl group) optionally having substituent(s), a C₂₋₁₀ alkenyl group (preferably a C₂₋₆ alkenyl group) optionally having substituent(s), or a C₂₋₁₀ alkynyl group (preferably a C₂₋₆ alkynyl group) optionally having substituent(s)];
p is 0 or 1; and
when Z₂ is a group represented by the formula: —N(R⁷)—, then R⁴ and R⁷ are optionally bonded to each other to form, together with an adjacent nitrogen atom, a nitrogen-containing heterocycle optionally having substituent(s) (wherein the nitrogen-containing heterocycle is preferably morpholine, piperidine or piperazine, and the substituent(s) of the nitrogen-containing heterocycle are 1 to 3 selected from

(i) a carboxyl group;

(ii) a carbamoyl group;

(iii) a C₁₋₆ alkoxy-carbonyl group; and

(iv) a C₁₋₆ alkyl group optionally substituted by 1 to 3 substituents selected from a carboxyl group, a carbamoyl group and a C₁₋₆ alkoxy-carbonyl group].

### 3) Type 3 a Group Represented by the Formula:

R⁸—Z₃—N(R⁹)—(Z)p-

wherein
R⁸ and R⁹ are each independently
(1) a hydrogen atom,
(2) a cyclic group (preferably a C₃₋₁₀ cycloalkyl group (e.g., cyclopropyl), a C₆₋₁₄ aryl group (e.g., phenyl) or a 5- or 6-membered aromatic heterocyclic group (e.g., pyridyl)) optionally having substituent(s),
(3) a C₁₋₁₀ alkyl group (preferably a C₁₋₆ alkyl group) optionally having substituent(s) (the substituent(s) is (are) 1 to 3 selected from a carboxyl group, a carbamoyl group and a C₁₋₆ alkoxy-carbonyl group),
(4) a C₂₋₁₀ alkenyl group (preferably a C₂₋₆ alkenyl group) optionally having substituent(s) (the substituent(s) is (are) 1 to 3 selected from A carboxyl group, a carbamoyl group and a C₁₋₆ alkoxy-carbonyl group), or
(5) a C₂₋₁₀ alkynyl group (preferably a C₂₋₆ alkynyl group) optionally having substituent(s) (the substituent(s) is (are) 1 to 3 selected from a carboxyl group, a carbamoyl group and a C₁₋₆ alkoxy-carbonyl group);
Z is a C₁₋₄ alkylene group;
Z₃ is —CO—, —CONH— or —SO₂—; and
p is 0 or 1.

### 4) Type 4 a Group Represented by the Formula:

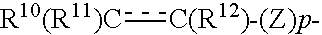

wherein
R₁₀, R¹¹ and R¹² are each independently
(1) a hydrogen atom,
(2) a cyclic group (preferably a C₆₋₁₄ aryl group (e.g., phenyl)) optionally having substituent(s) (the substituent(s) is (are) 1 to 3 selected from

(i) a carboxyl group;

(ii) a carbamoyl group;

(iii) a C₁₋₆ alkyl group optionally substituted by a carboxyl group;

(iv) a C₁₋₆ alkoxy group optionally substituted by a carboxyl group; and

(v) an aromatic heterocyclic group (e.g., tetrazolyl)),
(3) a C₁₋₁₀ alkyl group (preferably a C₁₋₆ alkyl group) optionally having substituent(s),
(4) a C₂₋₁₀ alkenyl group (preferably a C₂₋₆ alkenyl group) optionally having substituent(s), or
(5) a C₂₋₁₀ alkynyl group (preferably a C₂₋₆ alkynyl group) optionally having substituent(s);
Z is a C₁₋₄ alkylene group;
[symbol] is a single bond or a double bond; and
p is 0 or 1.

### 5) Type 5 a Group Represented by the Formula:

R¹³O—N═C(R¹⁴)—(Z)p-

wherein
R¹³ and R¹⁴ are each independently
(1) a hydrogen atom,
(2) a cyclic group (preferably a C₆₋₁₄ aryl group (e.g., phenyl)) optionally having substituent(s) (the substituent(s) is (are) 1 to 3 selected from

(i) a carboxyl group;

(ii) a carbamoyl group;

(iii) a C₁₋₆ alkyl group optionally substituted by a carboxyl group;

(iv) a C₁₋₆ alkoxy group optionally substituted by a carboxyl group; and

(v) an aromatic heterocyclic group (e.g., tetrazolyl)),
(3) a C₁₋₁₀ alkyl group (preferably a C₁₋₆ alkyl group) optionally having substituent(s),
(4) a C₂₋₁₀ alkenyl group (preferably a C₂₋₆ alkenyl group) optionally having substituent(s), or
(5) a C₂₋₁₀ alkynyl group (preferably a C₂₋₆ alkynyl group) optionally having substituent(s);
Z is a C₁₋₄ alkylene group; and
p is 0 or 1.

Preferable embodiment of Rc is
(1) a C₁₋₆ alkyl group optionally substituted by 1 to 3 substituents selected from

(i) a hydroxy group,

(ii) a C₆₋₁₄ aryl group (e.g., phenyl) optionally substituted by 1 to 3 substituents selected from
(a) a carboxyl group,
(b) a hydroxy group,
(c) a C₁₋₆ alkoxy group optionally substituted by 1 to 3 substituents selected from
   (A) a C₁₋₆ alkoxy group,
   (B) a carbamoyl group optionally mono- or di-substituted by a C₁₋₆ alkyl group optionally substituted by a carbamoyl group, and
   (C) a carboxyl group, and
(d) a carbamoyl group optionally mono- or di-substituted by a C₁₋₆ alkyl group optionally substituted by a hydroxy group,

(iii) a C₆₋₁₄ aryloxy group (e.g., phenoxy) optionally substituted by 1 to 3 substituents selected from
(a) a carboxyl group,
(b) a carbamoyl group,
(c) a C₁₋₆ alkyl group optionally substituted by a carboxyl group, and
(d) a C₁₋₄ alkylenedioxy group,

(iv) an aromatic heterocyclic group (e.g., imidazolyl, thienyl), and

(v) an amino group optionally mono- or di-substituted by substituent(s) selected from
(a) a C₁₋₆ alkyl group optionally substituted by 1 to 3 substituents selected from
   (A) a C₆₋₁₄ aryl group (e.g., phenyl), and
   (B) a carbamoyl group, and
(b) a C₆₋₁₄ aryl group (e.g., phenyl);
(2) a carbamoyl group optionally mono- or di-substituted by a C₁₋₆ alkyl optionally substituted by a C₆₋₁₄ aryl group (e.g., phenyl); or
(3) a carbamoyl group optionally mono- or di-substituted by an aromatic heterocyclic group (e.g., pyridyl).

Another preferable embodiment of Rc is
(1) an optionally substituted C₁₋₆ alkyl group;
(2) an optionally substituted C₆₋₁₄ aryl group;
(3) an optionally substituted C₂₋₆ alkenyl group;
(4) an optionally substituted C₁₋₆ alkyl-carbonyl group; or
(5) an optionally substituted carbamoyl group;
and preferably a C₁₋₆ alkyl group optionally substituted by 1 to 3 substituents selected from

(i) an optionally substituted C₆₋₁₄ aryl group, and

(ii) an optionally substituted C₁₋₆ alkoxy group.

Specifically,
(1) a C₁₋₆ alkyl group optionally substituted by 1 to 3 substituents selected from

(i) a hydroxy group,

(ii) a C₆₋₁₄ aryl group (e.g., phenyl) optionally substituted by 1 to 3 substituents selected from
(a) a carboxyl group,
(b) a hydroxy group,
(c) a C₁₋₆ alkyl group optionally substituted by 1 to 3 substituents selected from
   (A) a hydroxy group, and
   (B) a halogen atom,
(d) a C₁₋₆ alkoxy group optionally substituted by 1 to 3 substituents selected from
   (A) a C₁₋₆ alkoxy group,
   (B) a carbamoyl group optionally mono- or di-substituted by substituent(s) selected from a C₁₋₆ alkyl group optionally substituted by a carbamoyl group, and a C₁₋₆ alkylsulfonyl group,
   (C) a carboxyl group,
   (D) a C₁₋₆ alkoxy-carbonyl group optionally substituted by a non-aromatic heterocyclic group (e.g., dioxolyl) optionally substituted by 1 to 3 substituents selected from an oxo group and a C₁₋₆ alkyl group,
   (E) a cyano group, and
   (F) a non-aromatic heterocyclic group (e.g., oxadiazolinyl) optionally substituted by an oxo group,
(e) a carbamoyl group optionally mono- or di-substituted by substituent(s) selected from
   (A) a C₁₋₆ alkyl group optionally substituted by a hydroxy group, and
   (B) a C₁₋₆ alkylsulfonyl group,
(f) a non-aromatic heterocyclic group (e.g., oxadiazolinyl) optionally substituted by an oxo group,
(g) an aromatic heterocyclic group (e.g., tetrazolyl),
(h) a C₁₋₆ alkoxy-carbonyl group optionally substituted by a non-aromatic heterocyclic group (e.g., dioxolyl) optionally substituted by 1 to 3 substituents selected from an oxo group and a C₁₋₆ alkyl group,
(i) a cyano group,
(j) a sulfamoyl group, and
(k) a halogen atom,

(iii) a C₆₋₁₄ aryloxy group (e.g., phenoxy) optionally substituted by 1 to 3 substituents selected from
(a) a carboxyl group,
(b) a carbamoyl group,
(c) a C₁₋₆ alkyl group optionally substituted by 1 to 3 substituents selected from a carboxyl group and a halogen atom,
(d) a C₁₋₄ alkylenedioxy group,
(e) a C₁₋₆ alkyl-carbonyl group, and
(f) a cyano group,

(iv) a C₃₋₁₀ cycloalkyl group (e.g., cyclopropyl, cyclohexyl),

(v) an aromatic heterocyclic group (e.g., imidazolyl, thienyl, pyridyl, oxazolyl, oxadiazolyl, benzimidazolyl) optionally substituted by 1 to 3 substituents selected from
(a) a C₆₋₁₄ aryl group (e.g., phenyl), and
(b) a C₁₋₆ alkyl group,

(vi) a non-aromatic heterocyclic group (e.g., morpholinyl, piperidinyl, oxazolidinyl) optionally substituted by 1 to 3 substituents selected from
(a) a carboxy group,
(b) a C₁₋₆ alkoxy-carbonyl group,
(c) a carbamoyl group optionally mono- or di-substituted by a C₁₋₆ alkyl group optionally substituted by 1 to 3 substituents selected from a hydroxy group and a carbamoyl group, and
(d) an oxo group,

(vii) a C₁₋₆ alkoxy group optionally substituted by a C₆₋₁₄ aryl group (e.g., phenyl) optionally substituted by a C₁₋₆ alkylsulfonyl group,

(viii) a C₁₋₆ alkylthio group,

(ix) a C₆₋₁₄ arylthio group (e.g., phenylthio),

(x) a C₆₋₁₄ arylsulfinyl group (e.g., phenylsulfinyl),

(xi) a C₆₋₁₄ arylsulfonyl group (e.g., phenylsulfonyl),

(xii) an amino group optionally mono- or di-substituted by substituent(s) selected from
(a) a C₁₋₆ alkyl group optionally substituted by 1 to 3 substituents selected from
   (A) a C₆₋₁₄ aryl group (e.g., phenyl), and
   (B) a carbamoyl group,
(b) a C₆₋₁₄ aryl group (e.g., phenyl),
(c) a C₁₋₆ alkyl-carbonyl group optionally substituted by 1 to 3 substituents selected from
   (A) a carboxyl group,
   (B) a C₁₋₆ alkoxy-carbonyl group,
   (C) a carbamoyl group optionally mono- or di-substituted by a C₃₋₁₀ cycloalkyl group, and
   (D) a non-aromatic heterbcyclylcarbonyl group (e.g., morpholinylcarbonyl),
(d) a carbamoyl group optionally mono- or di-substituted by a C₁₋₆ alkyl group optionally substituted by 1 to 3 substituents selected from
   (A) a carboxyl group,
   (B) a C₁₋₆ alkoxy-carbonyl group; and
   (C) a carbamoyl group,
(e) a C₆₋₁₄ aryl-carbonyl group optionally substituted by a C₁₋₆ alkoxy group, and
(f) a C₃₋₁₀ cycloalkyl-carbonyl group,

(xiii) a cyano group,

(xiv) a carboxyl group, and

(xv) a carbamoyl group optionally mono- or di-substituted by substituent(s) selected from a C₁₋₆ alkyl group and a C₆₋₁₄ aryl group (e.g., phenyl);
(2) a C₆₋₁₄ aryl group (e.g., phenyl);
(3) a C₂₋₆ alkenyl group optionally substituted by a C₆₋₁₄ aryl group (e.g., phenyl);
(4) a C₁₋₆ alkyl-carbonyl group;
(5) a carbamoyl group optionally mono- or di-substituted by a C₁₋₆ alkyl optionally substituted by a C₆₋₁₄ aryl group (e.g., phenyl); or
(6) a carbamoyl group optionally mono- or di-substituted by an aromatic heterocyclic group (e.g., pyridyl);

More preferably a C₁₋₆ alkyl group optionally substituted by 1 to 3 substituents selected from

(i) a C₆₋₁₄ aryl group (e.g., phenyl) optionally substituted by a carboxyl group, and

(ii) a C₁₋₆ alkoxy group optionally substituted by a C₆₋₁₄ aryl group (e.g., phenyl) optionally substituted by a C₁₋₆ alkylsulfonyl group.

Ring B optionally further has substituent(s) (preferably 1 to 3 substituents), besides Rc, at substitutable position(s). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

As preferable substituents of ring B,
(1) a C₁₋₆ alkyl group optionally substituted by a hydroxy group;
   and the like can be mentioned.

X is a bond or a spacer having 1 to 6 atoms in the main chain.

The “main chain” of the “spacer having 1 to 6 atoms in the main chain” for X is a divalent straight chain connecting ring A (bonded at U) and Ra, and the atom number of the main chain is counted such that the number of atoms in the main chain will be minimum. The “main chain” consists of 1 to 6 atoms selected from a carbon atom and a heteroatom (e.g., oxygen atom, sulfur atom, nitrogen atom and the like), and may be saturated or unsaturated. Also, S may be oxidized.

As specific examples of the “spacer having 1 to 6 atoms in the main chain”, for example, a straight chain C₁₋₆ alkylene group, —X¹—NH—X²—, —X¹—O—X²— or —X¹—S—X²— (wherein, X¹ and X² are the same or different and each is a bond or a straight chain C₁₋₅ alkylene group, provided that when both X¹ and X² are straight chain C₁₋₅ alkylene groups, then the total of the carbon number of the straight chain C₁₋₅ alkylene group for X¹ and the carbon number of the straight chain C₁₋₅ alkylene group for X² should be not more than 5, and S may be oxidized) can be mentioned.

As the “straight chain C₁₋₆ alkylene group”, for example, —CH₂—, —CH₂C₂—, —CH₂CH₂CH₂—, —CH₂CH₂CH₂CH₂—, —CH₂CH₂CH₂CH₂CH₂—, and —CH₂CH₂CH₂CH₂CH₂CH₂— can be mentioned.

As the “straight chain C₁₋₅ alkylene group” for X¹ or X², for example, —CH₂—, —CH₂CH₂—, —CH₂CH₂CH₂—, —CH₂CH₂CH₂CH₂—, and —CH₂CH₂CH₂CH₂CH₂— can be mentioned.

The “spacer having 1 to 6 atoms in the main chain” optionally has substituent(s) (preferably 1 to 3 substituents) at substitutable position(s) (substitutable at the carbon atom(s) and nitrogen atom(s) constituting the main chain). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

X is preferably a bond or a straight chain C₁₋₆ alkylene group optionally having substituent(s), more preferably a bond, or a group represented by the formula: —(R¹)C(R²)— (wherein R¹ and R² are each independently a hydrogen atom or a C₁₋₃ alkyl group), further more preferably a bond. As used herein, as the “C₁₋₃ alkyl group” for R¹ or R², for example, methyl, ethyl, propyl and isopropyl can be mentioned.

Y is a spacer having 1 to 6 atoms in the main chain.

The “main chain” of the “spacer having 1 to 6 atoms in the main chain” for Y is a divalent straight chain connecting ring A (bonded at W) and ring B (bonded at the nitrogen atom). As the “spacer having 1 to 6 atoms in the main chain”, for example, those similar to the “spacer having 1 to 6 atoms in the main chain” for X can be mentioned.

Y is preferably —CO—, —CH₂—, —CH₂CO— or —SO₂—, more preferably —CO— or —CH₂—, further more preferably —CO—.

U, V and W are each independently C or N. Provided that when any one of U, V and W is N, then the others should be C.

Preferably, U is N, and both V and W are C.

In compound (I), the ring A-constituting atom (U) to which X is bonded and the ring A-constituting atom (V) to which Rb is bonded are adjacent to each other, and the ring A-constituting atom (V) to which Rb is bonded and the ring A-constituting atom (W) to which Y is bonded are adjacent to each other. Also, in compound (I′), the ring A-constituting atom (U) to which R is bonded and the ring A-constituting atom (V) to which R′ is bonded are adjacent to each other, and the ring A-constituting atom (V) to which R′ is bonded and the ring A-constituting atom (W) to which Y is bonded are adjacent to each other.

m and n are each independently 1 or 2.

Preferably, m and n are each independently 1, more preferably, both m and n are 1.

R, R′ and R″ are each independently a substituent.

As the “substituent” for R, R′ or R″, for example, an “optionally substituted hydrocarbon group”, an “optionally substituted heterocyclic group”, an “optionally substituted hydroxy”, an “optionally substituted amino group”, an “optionally substituted mercapto group”, a “cyano group”, a “nitro group”, an “acyl group”, a “halogen atom” and the like can be mentioned.

As the aforementioned “halogen atom”, for example, a fluorine atom, a chlorine atom, a bromine atom and an iodine atom can be mentioned.

As the “hydrocarbon group” of the aforementioned “optionally substituted hydrocarbon group”, for example, those similar to the “hydrocarbon group optionally having substituent(s)” of the “hydrocarbon group optionally containing heteroatom(s) as the constituting atom(s), which optionally has substituent(s)” for Rc can be mentioned.

As the “heterocyclic group” of the aforementioned “optionally substituted heterocyclic, group”, for example, an aromatic heterocyclic group and a non-aromatic heterocyclic group can be mentioned. As the aromatic heterocyclic group and non-aromatic heterocyclic group, for example, those exemplified as the “cyclic group” of the “cyclic group optionally having substituent(s)” for Ra or Rb can be mentioned.

The “heterocyclic group” of the aforementioned “optionally substituted heterocyclic group” optionally has substituent(s) (preferably 1 to 3 substituents) at substitutable position(s). As such substituents, for example, those similar to the substituents which the “cyclic group” of the “cyclic group optionally having substituent(s)” for Ra or Rb optionally has, can be mentioned. When the number of the substituents is not less than 2, respective substituents may be the same or different.

As the aforementioned “optionally substituted hydroxy group”, for example, a hydroxy group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group, a 5- or 6-membered aromatic heterocyclic group and a fused aromatic heterocyclic group, each of which optionally has substituent(s), and the like can be mentioned.

As used here, as the C₁₋₁₀ alkyl group, for example, those similar to the “C₁₋₁₀ alkyl group” of the “C₁₋₁₀ alkyl group optionally having substituent(s)” for Ra or Rb can be mentioned.

As the C₂₋₁₀ alkenyl group, for example, those similar to the “C₂₋₁₀ alkenyl group” of the “C₂₋₁₀ alkenyl group optionally having substituent(s)” for Ra or Rb can be mentioned.

As the C₃₋₁₀ cycloalkyl group, C₃₋₁₀ cycloalkenyl group and C₆₋₁₄ aryl group, for example, those exemplified as the “cyclic group” of the “cyclic group optionally having substituent(s)” for Ra or Rb can be mentioned.

As the C₇₋₁₃ aralkyl group and C₈₋₁₃ arylalkenyl group, for example, those exemplified as the “hydrocarbon group” of the “hydrocarbon group optionally containing heteroatom(s) as the constituting atom(s), which optionally has substituent(s)” for Rc can be mentioned.

As the C₁₋₆ alkyl-carbonyl group, for example, acetyl, propanoyl, butanoyl, isobutanoyl, pentanoyl, isopentanoyl, hexanoyl and the like can be mentioned.

As the 5- or 6-membered aromatic heterocyclic group, for example, a 5- or 6-membered cyclic group, from among the “aromatic heterocyclic group” exemplified as the “cyclic group” of the “cyclic group optionally having substituent(s)” for Ra or'Rb, can be mentioned.

As the fused aromatic heterocyclic group, for example, a fused cyclic group, from among the “aromatic heterocyclic group” exemplified as the.“cyclic group” of the “cyclic group optionally having substituent(s)” for Ra or Rb, can be mentioned.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group, C₁₋₆ alkyl-carbonyl group, 5- or 6-membered aromatic heterocyclic group and fused aromatic heterocyclic group optionally have substituent(s) (preferably 1 to 3 substituents) at substitutable position(s).

As the substituents of the C₁₋₁₀ alkyl group, C₂₋₁₀ alkenyl group and C₁₋₆ alkyl-carbonyl group, for example, those similar to the substituents which the “C₁₋₁₀ alkyl group” of the “C₁₋₁₀ alkyl group optionally having substituent(s)” for Ra or Rb optionally has, can be mentioned. When the number of the substituents is not less than 2, respective substituents may be the same or different.

As the substituents of the C₃₋₁₀ cycloalkyl group, C₃₋₁₀ cycloalkenyl group, C₆₋₁₄ aryl group, C₇₋₁₃ aralkyl group, C₈₋₁₃ arylalkenyl group, 5- or 6-membered aromatic heterocyclic group and fused aromatic heterocyclic group, for example, those similar to the substituents which “cyclic group” of the “cyclic group optionally having substituent(s)” for Ra or Rb optionally has, can be mentioned. When the number of the substituents is not less than 2, respective substituents may be the same or different.

As the aforementioned “optionally substituted mercapto group”, for example, a mercapto group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group, a 5- or 6-membered aromatic heterocyclic group and a fused aromatic heterocyclic group, each of which optionally has substituent(s), and the like can be mentioned.

As the substituents, those similar to the substituents of the aforementioned “optionally substituted hydroxy group” can be mentioned.

As the aforementioned “optionally substituted amino group”, for example, an amino group optionally substituted by one or two substituents selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group and a C₈₋₁₃ arylalkenyl group, each of which optionally has substituent(s); an acyl group and the like can be mentioned.

As used here, as the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group, those exemplified as the substituents of the aforementioned “optionally substituted hydroxy group” can be mentioned.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group optionally have substituent(s) (preferably 1 to 3 substituents) at substitutable position(s).

As the substituents of the C₁₋₁₀ alkyl group and C₂₋₁₀ alkenyl group, for example, those similar to the substituents which the “C₁₋₁₀ alkyl group” of the “C₁₋₁₀ alkyl group optionally having substituent(s)” for Ra or Rb optionally has, can be mentioned. When the number of the substituents is not less than 2, respective substituents may be the same or different.

As the substituents of the C₃₋₁₀ cycloalkyl group, C₃₋₁₀ cycloalkenyl group, C₆₋₁₄ aryl group, C₇₋₁₃ aralkyl group and C₈₋₁₃ arylalkenyl group, for example, those similar to the substituents which “cyclic group” of the “cyclic group optionally having substituent(s)” for Ra or Rb optionally has, can be mentioned. When the number of the substituents is not less than 2, respective substituents may be the same or different.

As the acyl group, for example, a group represented by the formula: —COR^{A}, —CO—OR^{A}, —SO₂R^{A}, —SOR^{A}, —CO —NR^{A}′R^{B}′, —CS— NR^{A}′R^{B}′ [wherein R^{A} is a hydrogen atom, an optionally substituted hydrocarbon group or an optionally substituted heterocyclic group, and R^{A}′ and R^{B}′ are the same or different and each is a hydrogen atom, an optionally substituted hydrocarbon group or an optionally substituted heterocyclic so group, or R^{A}′ and R^{B}′ optionally form, together with the adjacent nitrogen atom, an optionally substituted nitrogen-containing heterocycle] and the like can be mentioned.

As the “optionally substituted hydrocarbon group” and “optionally substituted heterocyclic group” for R^{A}, R^{A}′ or R^{B}′, those exemplified as the “substituent” for R, R′ or R″ can be mentioned.

As the “nitrogen-containing heterocycle” of the “optionally substituted nitrogen-containing heterocycle” formed by R^{A}′ and R⁸′ together with the adjacent nitrogen atom, for example, a 5- to 7-membered nitrogen-containing heterocycle containing, as a ring-constituting atom besides carbon atoms, at least one nitrogen atom and optionally further containing one or two heteroatoms selected from an oxygen atom, a sulfur atom and a nitrogen atom can be mentioned. As preferable examples of the nitrogen-containing heterocycle, pyrrolidine, imidazolidine, pyrazolidine, piperidine, piperazine, morpholine, thiomorpholine, oxopiperazine and the like can be mentioned.

The nitrogen-containing heterocycle optionally has substituent(s) (preferably 1 to 3 substituents, more preferably 1 or 2 substituents) at substitutable position(s). As such substituents, those similar to the substituents which the “cyclic group” of the “cyclic group optionally having substituent(s)” for Ra or Rb optionally has, can be mentioned. When the number of the substituents is not less than 2, respective substituents may be the same or different.

As preferable examples of the “acyl group”,
(1) a formyl group;
(2) a carboxyl group;
(3) a carbamoyl group;
(4) a C₁₋₆ alkyl-carbonyl group;
(5) a C₁₋₆ alkoxy-carbonyl group optionally substituted by 1 to 3 substituents selected from a carboxyl group, a carbamoyl group, a thiocarbamoyl group, a C₁₋₆ alkoxy-carbonyl group and a C₁₋₆ alkyl-carbonyloxy group (e.g., methoxycarbonyl, ethoxycarbonyl, propoxycarbonyl, tert-butoxycarbonyl; carboxymethoxycarbonyl, carboxyethoxycarbonyl, carboxybutoxycarbonyl; carbamoylmethoxycarbonyl; thiocarbamoylmethoxycarbonyl; ethoxycarbonylmethoxycarbonyl, ethoxycarbonylethoxycarbonyl, methoxycarbonylbutoxycarbonyl, ethoxycarbonylbutoxycarbonyl; tert-butylcarbonyloxymethoxycarbonyl);
(6) a C₃₋₁₀ cycloalkyl-carbonyl group (e.g., cyclopentylcarbonyl, cyclohexylcarbonyl);
(7) a C₆₋₁₄ aryl-carbonyl group (e.g., benzoyl, 1-naphthyl, 2-naphthyl) optionally substituted by 1 to 3 substituents selected from a halogen atom, a cyano group, a C₁₋₆ alkyl group optionally substituted by 1 to 3 halogen atoms, a C₁₋₆ alkoxy group, a carboxyl group, a C₁₋₆ alkoxy-carbonyl group, an aromatic heterocyclic group (e.g., tetrazolyl, oxadiazolyl), a non-aromatic heterocyclic group (e.g., oxooxadiazolyl) and a carbamoyl group;
(8) a C₆₋₁₄ aryloxy-carbonyl group (e.g., phenyloxycarbonyl, naphthyloxycarbonyl) optionally substituted by 1 to 3 substituents selected from a carboxyl group, a C₁₋₆ alkoxy-carbonyl group and a carbamoyl group;
(9) a C₇₋₁₃ aralkyloxy-carbonyl group optionally substituted by 1 to 3 substituents selected from a carboxyl group, a carbamoyl group, a thiocarbamoyl group, a C₁₋₆ alkoxy-carbonyl group, a halogen atom, a cyano group, a nitro group, a C₁₋₆ alkoxy group, a C₁₋₆ alkylsulfonyl group and a C₁₋₆ alkyl group (e.g., benzyloxycarbonyl, phenethyloxycarbonyl; carboxybenzyloxycarbonyl; methoxycarbonylbenzyloxycarbonyl; biphenylylmethoxycarbonyl);
(10) a carbamoyl group mono- or di-substituted by a C₁₋₆ alkyl group optionally substituted by 1 to 3 substituents selected from a halogen atom and a C₁₋₆ alkoxy group (e.g., methylcarbamoyl, ethyl carbamoyl, dimethylcarbamoyl, diethylcarbamoyl, ethylmethylcarbamoyl, propylcarbamoyl, isopropylcarbamoyl, butylcarbamoyl, isobutylcarbamoyl, trifluoroethylcarbamoyl, N-methoxyethyl-N-methylcarbamoyl);
(11) a C₁₋₆ alkylsulfonyl group optionally substituted by 1 to 3 substituents selected from a carboxyl group, a carbamoyl group and a C₁₋₆ alkoxy-carbonyl group (e.g., methylsulfonyl, carboxymethylsulfonyl);
(12) a C₁₋₆ alkylsulfinyl group (e.g., methylsulfinyl);
(13) a thiocarbamoyl group;
(14) a C₇₋₁₃ aralkyl-carbonyl group (e.g., benzylcarbonyl, phenethylcarbonyl);
(15) an aromatic heterocyclyl (e.g., furyl, thienyl, oxazolyl, thiazolyl, isoxazolyl, isothiazolyl, pyrazolyl, pyridyl, pyrazinyl, benzofuryl, benzothienyl, quinoxalinyl)-carbonyl (e.g., furylcarbonyl, thienylcarbonyl, thiazolylcarbonyl, pyrazolylcarbonyl, pyridylcarbonyl, pyrazinylcarbonyl, benzofurylcarbonyl, benzothienylcarbonyl, quinoxalinylcarbonyl) optionally substituted by 1 to 3 substituents selected from a C₁₋₆ alkyl group, a C₆₋₁₄ aryl group, a C₇₋₁₃ aralkyl group, a C₁₋₆ alkoxy group, a carboxyl group, a C₁₋₆ alkoxy-carbonyl group and a carbamoyl group;
   and the like can be mentioned.

Among Compounds (I′), compound (I) is preferred.

Preferable compound (I) is as follows:

### [Compound A]

A compound wherein
ring A is a 5-membered aromatic heterocycle (preferably pyrrole, pyrazole, 1,2,3-triazole, imidazole or thiophene, more preferably pyrrole, pyrazole, 1,2,3-triazole or imidazole, particularly preferably imidazole or pyrrole, most preferably imidazole) optionally having substituent(s) [the substituent(s) is (are) 1 to 3 selected from the following (1) to (3):
(1) a C₁₋₆ alkyl group optionally substituted by a C₁₋₆ alkoxy group;
(2) a C₆₋₁₄ aryl group (e.g., phenyl); and
(3) a C₁₋₆ alkyl-carbonyl group];
U is N or C (preferably N);
both V and W are C;
Ra and Rb are each independently a cyclic group (preferably a C₆₋₁₄ aryl group (e.g., phenyl), a 5- or 6-membered aromatic heterocyclic group (e.g., pyridyl, thienyl, thiazolyl) or a C₃₋₁₀ cycloalkyl group optionally condensed with a benzene ring (e.g., cyclopropyl, cyclohexyl, indanyl, tetrahydronaphthyl)) optionally having substituent(s), or a C₁₋₁₀ alkyl group (preferably a C₁₋₆ alkyl group) optionally having substituent(s)
[more preferably a C₆₋₁₄ aryl group (e.g., phenyl) optionally having substituent(s), a 5- or 6-membered aromatic heterocyclic group (e.g., pyridyl, thienyl, thiazolyl) optionally having substituent(s), or a C₃₋₁₀ cycloalkyl group optionally condensed with a benzene ring (e.g., cyclopropyl, cyclohexyl, indanyl, tetrahydronaphthyl), which optionally has substituent(s), further more preferably a C₆₋₁₄ aryl group (e.g., phenyl) optionally having substituent(s), or a C₃₋₁₀ cycloalkyl group condensed with a benzene ring (e.g., indanyl, tetrahydronaphthyl), which optionally has substituent(s)]
   [the substituent(s) is (are) 1 to 3 selected from the following (1) to (17):
(1) a halogen atom;
(2) a C₁₋₆ alkoxy group optionally substituted by 1 to 3 substituents selected from

(i) a carboxyl group,

(ii) a hydroxy group,

(iii) a C₁₋₆ alkoxy group,

(iv) a C₆₋₁₄ aryl group (e.g., phenyl),

(v) a C₁₋₆ alkoxy-carbonyl group,

(vi) a C₁₋₆ alkylsulfonyl group,

(vii) a carbamoyl group, and

(viii) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group, and

(v) a non-aromatic heterocyclylcarbonyl group (the non-aromatic heterocycle may be oxidized; e.g., morpholinylcarbonyl);
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl), and
(c) an amino group optionally mono- or di-substituted by a C₁₋₆ alkyl-carbonyl group, and

(iii) a non-aromatic heterocyclic group (the non-aromatic heterocyclic group may be oxidized; e.g., tetrahydrofuryl) optionally substituted by a hydroxy group;
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom, a hydroxy group and a carbamoyl group;
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl) optionally substituted by 1 to 3 substituents selected from

(i) a C₁₋₆ alkyl group optionally substituted by a hydroxy group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group, and

(vi) a carbamoyl group optionally mono- or di-substituted by a C₁₋₆ alkyl group optionally substituted by 1 to 3 substituents selected from a hydroxy group and a carbamoyl group;
(11) a non-aromatic heterocyclyloxy group (the non-aromatic heterocycle may be oxidized; e.g., 1,1-dioxidotetrahydrothiopyranyloxy);
(12) a C₂₋₆ alkoxy-carbonyl group;
(13) a carboxyl group;
(14) a non-aromatic heterocyclylcarbonyl group (the non-aromatic heterocycle may be oxidized; e.g., morpholinylcarbonyl);
(15) a C₁₋₄ alkylenedioxy group optionally substituted by a halogen atom;
(16) a C₆₋₁₄ aryl group (e.g., phenyl) optionally substituted by a C₁₋₆ alkoxy group; and
(17) an aromatic heterocyclic group (e.g., thienyl, tetrazolyl)]
[further more preferably, Ra is
(A) a C₆₋₁₄ aryl group (e.g., phenyl) optionally substituted by 1 to 3 substituents selected from
(1) a halogen atom;
(2) a C₁₋₆ alkoxy group optionally substituted by 1 to 3 substituents selected from

(i) a hydroxy group,

(ii) a C₁₋₆ alkoxy group,

(iii) a C₆₋₁₄ aryl group (e.g., phenyl),

(iv) a C₁₋₆ alkylsulfonyl group, and

(v) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group, and

(v) a non-aromatic heterocyclylcarbonyl group (the non-aromatic heterocycle may be oxidized; e.g., morpholinylcarbonyl);
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group. (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl), and
(c) an amino group optionally mono- or di-substituted by a C₁₋₆ alkyl-carbonyl group, and

(iii) a non-aromatic heterocyclic group (the non-aromatic heterocyclic group may be oxidized; e.g., tetrahydrofuryl) optionally substituted by a hydroxy group;
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom and a hydroxy group;
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl) optionally substituted by 1 to 3 substituents selected from

(i) a C₁₋₆ alkyl group optionally substituted by a hydroxy group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group, and

(vi) a carbamoyl group optionally mono- or di-substituted by a C₁₋₆ alkyl group optionally substituted by 1 to 3 substituents selected from a hydroxy group and a carbamoyl group;
(11) a non-aromatic heterocyclyloxy group (the non-aromatic heterocycle may be oxidized; 1,1-dioxidotetrahydrothiopyranyloxy);
(12) a C₁₋₆ alkoxy-carbonyl group;
(13) a carboxyl group;
(14) a non-aromatic heterocyclylcarbonyl group (the non-aromatic heterocycle may be oxidized; e.g., morpholinylcarbonyl);
(15) a C₁₋₄ alkylenedioxy group optionally substituted by a halogen atom; and
(16) an aromatic heterocyclic group (e.g., tetrazolyl);
(B) a 5 or 6-membered aromatic heterocyclic group (e.g., pyridyl, thienyl);
(C) C₃₋₁₀ cycloalkyl group condensed with a benzene ring (e.g., indanyl, tetrahydronaphthyl); or
(D) a C₁₋₆ alkyl group optionally substituted by 1 to 3 substituents selected from
(1) a carbamoyl group optionally mono- or di-substituted by a C₁₋₆ alkyl group optionally substituted by 1 to 3 substituents selected from a hydroxy group and a carbamoyl group;
(2) a C₆₋₁₄ aryl group (e.g., phenyl) optionally substituted by a C₁₋₆ alkoxy group; and.
(3) an aromatic heterocyclic group (e.g., thienyl); and
Rb is
(A) a C₆₋₁₄ aryl group (e.g., phenyl) optionally substituted by 1 to 3 substituents selected from
(1) a halogen atom;
(2) a hydroxy group; and
(3) a C₁₋₆ alkoxy group optionally substituted by 1 to 3 substituents selected from

(i) a C₆₋₁₄ aryl group (e.g., phenyl),

(ii) a carboxyl group,

(iii) a C₁₋₆ alkoxy-carbonyl group, and

(iv) a carbamoyl group;
(B) a 5 or 6-membered aromatic heterocyclic group (e.g., pyridyl, thiazolyl, thienyl);
(C) a C₁₋₆ alkyl group (e.g., methyl, propyl); or
(D) a C₃₋₁₀ cycloalkyl group (e.g., cyclopropyl, cyclohexyl)];
X is a bond, or a group represented by the formula: —(R¹)C(R²)— (where R¹ and R² are each independently a hydrogen atom or a C₁₋₃ alkyl group) (preferably a bond);
Y is —CO—, —CH₂—, —CH₂CO— or —SO₂— (preferably —CO— or —CH₂—, more preferably —CO—);
Rc is

### 1) Type 1 a Group Represented by the Formula:

R³—(Z₁)q-(Z)p-

wherein
R³ is
(1) a hydrogen atom,
(2) a cyclic group (preferably a C₆₋₁₄ aryl group (e.g., phenyl) or a 5- or 6-membered aromatic heterocyclic group (e.g., imidazolyl, thienyl)) optionally having substituent(s) (the substituent(s) is (are) 1 to 3 selected from

(i) a carboxyl group;

(ii) a C₁₋₆ alkoxy group optionally substituted by 1 to 3 substituents selected from.
(a) a C₁₋₆ alkoxy group,
(b) a carbamoyl group optionally mono- or di-substituted by a C₁₋₆ alkyl group optionally substituted by a carbamoyl group, and
(c) a carboxyl group;

(iii) a hydroxy group;

(iv) a carbamoyl group optionally mono- or di-substituted by substituent(s) selected from
(a) a C₁₋₆ alkyl group optionally substituted by a hydroxy group, and
(b) a C₁₋₆ alkylsulfonyl group;

(v) a C₁₋₆ alkyl group optionally substituted by a carboxyl group;

(vi) a C₁₋₄ alkylenedioxy group;

(vii) an amino group optionally mono- or di-substituted by a C₁₋₆ alkyl-carbonyl group;

(viii) a sulfamoyl group optionally mono- or di-substituted by a C₁₋₆ alkyl-carbonyl group;

(ix) an aromatic heterocyclic group (e.g., tetrazolyl); and

(x) a non-aromatic heterocyclic group (e.g., dihydroxadiazolyl) optionally substituted by 1 to 3 substituents selected from an oxo group and a thioxo group),
(3) a C₁₋₁₀ alkyl group (preferably a C₁₋₆ alkyl group) optionally having substituent(s) (the substituent(s) is (are) 1 to 3 selected from a hydroxy group, a carboxyl group and a carbamoyl group),
(4) a C₂₋₁₀ alkenyl group (preferably a C₂₋₆ alkenyl group) optionally having substituent(s) (the substituent(s) is (are) 1 to 3 selected from a hydroxy group, a carboxyl group and a carbamoyl group), or
(5) a C₂₋₁₀ alkynyl group (preferably a C₂₋₆ alkynyl group) optionally having substituent(s) (the substituent(s) is (are) 1 to 3 selected from a hydroxy group, a carboxyl group and a carbamoyl group);
Z is a C₁₋₄ alkylene group;
Z₁ is —CO—, —O—, —S—, —S(O)— or —S(O)₂—; and
p and q are each independently 0 or 1;
2) Type 2 a group represented by the formula:

R⁴—Z₂—(R⁵)C(R⁶)—(Z)p-

wherein
R⁴ is
(1) a hydrogen atom,
(2) a cyclic group (preferably a C₆₋₁₄ aryl group (e.g., phenyl) or a 5- or 6-membered aromatic heterocyclic group (e.g., pyridyl)) optionally having substituent(s),
(3) a C₁₋₁₀ alkyl group (preferably a C₁₋₆ alkyl group) optionally having substituent(s) (the substituent(s) is (are) 1 to 3 selected from a carbamoyl group and a C₆₋₁₄ aryl group (e.g., phenyl)),
(4) a C₂₋₁₀ alkenyl group (preferably a C₂₋₆ alkenyl group) optionally having substituent(s) (the substituent(s) is (are) 1 to 3 selected from a carbamoyl group and a C₆₋₁₄ aryl group (e.g., phenyl)), or
(5) a C₂₋₁₀ alkynyl group (preferably a C₂₋₆ alkynyl group) optionally having substituent(s) (the substituent(s) is (are) 1 to 3 selected from a carbamoyl group and a C₆₋₁₄ aryl group (e.g., phenyl));
R⁵ and R⁶ are each independently
(1) a hydrogen atom,
(2) a cyclic group (preferably a C₃₋₁₀ cycloalkyl group (e.g., cyclopropyl) or a C₆₋₁₄ aryl group (e.g., phenyl)) optionally having substituent(s),
(3) a C₁₋₁₀ alkyl group (preferably a C₁₋₆ alkyl group) optionally having substituent(s) (the substituent(s) is (are) 1 to 3 selected from

(i) a C₁₋₆ alkoxy group;

(ii) an amino group optionally mono- or di-substituted by substituent(s) selected from a C₁₋₆ alkyl group and a C₁₋₆ alkyl-carbonyl group;

(iii) a carboxyl group; and

(iv) a C₁₋₆ alkoxy-carbonyl group),
(4) a C₂₋₁₀ alkenyl group (preferably a C₂₋₆ alkenyl group) optionally having substituent(s), or
(5) a C₂₋₁₀ alkynyl group (preferably a C₂₋₆ alkynyl group) optionally having substituent(s), or
R⁵ and R⁶ in combination form an oxo group;
Z is a C₁₋₄ alkylene group;
Z₂ is —O—, or a group represented by the formula: —N(R⁷)— [wherein R⁷ is a hydrogen atom, a cyclic group (preferably a C₃₋₁₀ cycloalkyl group (e.g., cyclopropyl), a C₆₋₁₄ aryl group (e.g., phenyl) or a 5- or 6-membered aromatic heterocyclic group (e.g., pyridyl)) optionally having substituent(s), a C₁₋₁₀ alkyl group (preferably a C₁₋₆ alkyl group) optionally having substituent(s), a C₂₋₁₀ alkenyl group (preferably a C₂₋₆ alkenyl group) optionally having substituent(s), or a C₂₋₁₀ alkynyl group (preferably a C₂₋₆ alkynyl group) optionally having substituent(s)];
p is 0 or 1; and
when Z₂ is a group represented by the formula: —N(R⁷)—, then R⁴ and R⁷ are optionally bonded to each other to form, together with an adjacent nitrogen atom, a nitrogen-containing heterocycle optionally having substituent(s) [wherein the nitrogen-containing heterocycle is preferably morpholine, piperidine or piperazine, and the substituent(s) of the nitrogen-containing heterocycle are 1 to 3 selected from

(i) a carboxyl group;

(ii) a carbamoyl group;

(iii) a C₁₋₆ alkoxy-carbonyl group; and

(iv) a C₁₋₆ alkyl group optionally substituted by 1 to 3 substituents selected from a carboxyl group, a carbamoyl group and a C₁₋₆ alkoxy-carbonyl group];

### 3) Type 3 a Group Represented by the Formula:

R⁸—Z₃—N (R⁹)—(Z)p-

wherein
R⁸ and R⁹ are each independently
(1) a hydrogen atom,
(2) a cyclic group (preferably a C₃₋₁₀ cycloalkyl group (e.g., cyclopropyl), a C₆₋₁₄ aryl group (e.g., phenyl) or a 5- or 6-membered aromatic heterocyclic group (e.g., pyridyl)) optionally having substituent(s),
(3) a C₁₋₁₀ alkyl group (preferably a C₁₋₆ alkyl group) optionally having substituent(s) (the substituent(s) is (are) 1 to 3 selected from a carboxyl group, a carbamoyl group and a C₁₋₆ alkoxy-carbonyl group),
(4) a C₂₋₁₀ alkenyl group (preferably a C₂₋₆ alkenyl group) optionally having substituent(s) (the substituent(s) is (are) 1 to 3 selected from a carboxyl group, a carbamoyl group and a C₁₋₆ alkoxy-carbonyl group), or
(5) a C₂₋₁₀ alkynyl group (preferably a C₂₋₆ alkynyl group) optionally having substituent(s) (the substituent(s) is (are) 1 to 3 selected from a carboxyl group, a carbamoyl group and a C₁₋₆ alkoxy-carbonyl group);
Z is a C₁₋₄ alkylene group;
Z₃ is —CO—, —CONH— or —SO₂—; and
p is 0 or 1;

### 4) Type 4 a Group Represented by the Formula:

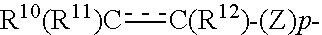

wherein
R¹⁰, R₁₁ and R¹² are each independently
(1) a hydrogen atom,
(2) a cyclic group (preferably a C₆₋₁₄ aryl group (e.g., phenyl)) optionally having substituent(s) (the substituent(s) is (are) 1 to 3 selected from

(i) a carboxyl group;

(ii) a carbamoyl group;

(iii) a C₁₋₆ alkyl group optionally substituted by a carboxyl group;

(iv) a C₁₋₆ alkoxy group optionally substituted by a carboxyl group; and

(v) an aromatic heterocyclic group (e.g., tetrazolyl)),
(3) a C₁₋₁₀ alkyl group (preferably a C₁₋₆ alkyl group) optionally, having substituent(s),
(4) a C₂₋₁₀ alkenyl group (preferably a C₂₋₆ alkenyl group) optionally having substituent(s), or
(5) a C₂₋₁₀ alkynyl group (preferably a C₂₋₆ alkynyl group) optionally having substituent(s);
Z is a C₁₋₄ alkylene group;
[symbol] is a single bond or a double bond; and
p is 0 or 1; or

### 5) Type 5 a Group Represented by the Formula:

R¹³O—N═C(R¹⁴)—(Z)p-

wherein
R¹³ and R¹⁴ are each independently
(1) a hydrogen atom,
(2) a cyclic group (preferably a C₆₋₁₄ aryl group (e.g., phenyl)) optionally having substituent(s) (the substituent(s) is (are) 1 to 3 selected from

(i) a carboxyl group;

(ii) a carbamoyl group;

(iii) a C₁₋₆ alkyl group optionally substituted by a carboxyl group;

(iv) a C₁₋₆ alkoxy group optionally substituted by a carboxyl group; and

(v) an aromatic heterocyclic group (e.g., tetrazolyl)),
(3) a C₁₋₁₀ alkyl group (preferably a C₁₋₆ alkyl group) optionally having substituent(s),
(4) a C₂₋₁₀ alkenyl group (preferably a C₂₋₆ alkenyl group) optionally having substituent(s), or
(5) a C₂₋₁₀ alkynyl group (preferably a C₂₋₆ alkynyl group) optionally having substituent(s);
Z is a C₁₋₄ alkylene group; and
p is 0 or 1;
[preferably, Rc is
(1) a C₁₋₆ alkyl group optionally substituted by 1 to 3 substituents selected from

(i) a hydroxy group,

(ii) a C₆₋₁₄ aryl group (e.g., phenyl) optionally substituted by 1 to 3 substituents selected from
(a) a carboxyl group,
(b) a hydroxy group,
(c) a C₁₋₆ alkoxy group optionally substituted by 1 to 3 substituents selected from
   (A) a C₁₋₆ alkoxy group,
   (B) a carbamoyl group optionally mono- or di-substituted by a C₁₋₆ alkyl group optionally substituted by a carbamoyl group, and
   (C) a carboxyl group, and
(d) a carbamoyl group optionally mono- or di-substituted by a C₁₋₆ alkyl group optionally substituted by a hydroxy group,

(iii) a C₆₋₁₄ aryloxy group (e.g., phenoxy) optionally substituted by 1 to 3 substituents selected from
(a) a carboxyl group,
(b) a carbamoyl group,
(c) a C₁₋₆ alkyl group optionally substituted by a carboxyl group, and
(d) a C₁₋₄ alkylenedioxy group,

(iv) an aromatic heterocyclic group (e.g., imidazolyl, thienyl), and

(v) an amino group optionally mono- or di-substituted by substituent(s) selected from
(a) a C₁₋₆ alkyl group optionally substituted by 1 to 3 substituents selected from
   (A) a C₆₋₁₄ aryl group (e.g., phenyl), and
   (B) a carbamoyl group, and
(b) a C₆₋₁₄ aryl group (e.g., phenyl);
(2) a carbamoyl group optionally mono- or di-substituted by a C₁₋₆ alkyl optionally substituted by a C₆₋₁₄ aryl group (e.g., phenyl); or
(3) a carbamoyl group optionally mono- or di-substituted by an aromatic heterocyclic group (e.g., pyridyl)]; and
m and n are each independently 1 or 2 (preferably 1, more preferably both m and n are 1).

### [Compound B]

A compound wherein
ring A is a 5 or 6-membered aromatic heterocycle (preferably pyrrole, pyrazole, triazole (1,2,3-triazole, 1,2,4-triazole), imidazole, thiophene or pyridine, more preferably a 5-membered aromatic heterocycle, further more preferably pyrrole, pyrazole, triazole (1,2,3-triazole, 1,2,4-triazole), imidazole or thiophene, still more preferably pyrrole, pyrazole, 1,2,3-triazole or imidazole, particularly preferably imidazole or pyrrole, most preferably imidazole) optionally having substituent(s)
[the substituent(s) is (are) 1 to 3 selected from the following (1) to (6):
(1) a halogen atom;
(2) a C₁₋₆ alkyl group optionally substituted by 1 to 3 substituents selected from

(a) a hydroxy group,

(b) a C₁₋₆ alkoxy group,

(c) an amino group,

(d) a carbamoyl group optionally mono- or di-substituted by a C₁₋₆ alkyl group optionally substituted by a hydroxy group,

(e) a C₆₋₁₄ aryl group (e.g., phenyl) optionally substituted by an aromatic heterocyclic group (e.g., pyrrolyl),

(f) an aromatic heterocyclic group (e.g., thiazolyl), and

(g) anon-aromatic heterocyclic group (e.g., morpholinyl);
(3) a C₆₋₁₄ aryl group (e.g., phenyl);
(4) a C₁₋₆ alkyl-carbonyl group;
(5) a C₁₋₆ alkoxy group; and
(6) a formyl group];
U is N or C (preferably N);
V is N or C (preferably C);
W is C;
Ra and Rb are each independently a cyclic group (preferably a C₆₋₁₄ aryl group (e.g., phenyl), a 5- or 6-membered aromatic heterocyclic group (e.g., pyridyl, thienyl, thiazolyl), a 5 or 6-membered non-aromatic heterocyclic group (e.g., pyrrolidinyl, piperidinyl, hexamethyleneiminyl, tetrahydrofuryl, tetrahydropyranyl, preferably a 5 or 6-membered non-aromatic nitrogen-containing heterocyclic group), or a C₃₋₁₀ cycloalkyl group optionally condensed with a benzene ring (e.g., cyclopropyl, cyclohexyl, indanyl, tetrahydronaphthyl)) optionally having substituent(s), or a C₁₋₁₀ alkyl group (preferably a C₁₋₆ alkyl group) optionally having substituent(s)
[more preferably a C₆₋₁₄ aryl group (e.g., phenyl) optionally having substituent(s), a 5- or 6-membered aromatic heterocyclic group (e.g., pyridyl, thienyl, thiazolyl) optionally having substituent(s), a 5 or 6-membered non-aromatic heterocyclic group (e.g., pyrrolidinyl, piperidinyl, hexamethyleneiminyl, tetrahydrofuryl, tetrahydropyranyl, preferably a 5 or 6-membered non-aromatic nitrogen-containing heterocyclic group) optionally having substituent(s), or a C₃₋₁₀ cycloalkyl group optionally condensed with a benzene ring (e.g., cyclopropyl, cyclohexyl, indanyl, tetrahydronaphthyl), which optionally has substituent(s), further more preferably a C₆₋₁₄ aryl group (e.g., phenyl) optionally having substituent(s), a 5 or 6-membered non-aromatic heterocyclic group (e.g., pyrrolidinyl, piperidinyl, hexamethyleneiminyl, tetrahydrofuryl, tetrahydropyranyl, preferably a 5 or 6-membered non-aromatic nitrogen-containing heterocyclic group) optionally having substituent(s), or a C₃₋₁₀ cycloalkyl group condensed with a benzene ring (e.g., indanyl, tetrahydronaphthyl), which optionally has substituent(s)]
[the substituent(s) is (are) 1 to 3 selected from the following (1) to (25):
(1) a halogen atom;
(2) a C₁₋₆ alkoxy group optionally substituted by 1 to 3 substituents selected from

(i) a carboxyl group,

(ii) a hydroxy group,

(iii) a C₁₋₆ alkoxy group,

(iv) a C₆₋₁₄ aryl group (e.g., phenyl),

(v) a C₁₋₆ alkoxy-carbonyl group,

(vi) a C₁₋₆ alkylsulfonyl group,

(vii) a carbamoyl group, and

(viii) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group,

(v) a non-aromatic heterocyclylcarbonyl group (the non-aromatic heterocycle may be oxidized; e.g., morpholinylcarbonyl),

(vi) a C₆₋₁₄ aryl group (e.g., phenyl) optionally substituted by a C₁₋₆ alkylsulfonyl group,

(vii) a C₃₋₁₀ cycloalkyl group (e.g., cyclopropyl);

(viii) an aromatic heterocyclic group (e.g., furyl) optionally substituted by 1 to 3 substituents selected from a carboxyl group and a C₁₋₆ alkoxy-carbonyl group,

(ix) a non-aromatic heterocyclic group (the non-aromatic heterocyclic group may be oxidized; e.g., tetrahydrofuryl) optionally substituted by a C₁₋₆ alkyl group, and

(x) a C₁₋₆ alkoxy group;
(4) an amino group optionally mono- or di-substituted by substituent(s) selected from

(i) a C₁₋₄₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) ‘a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group. (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and
(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl),
(c) an amino group optionally mono- or di-substituted by a C₁₋₆ alkyl-carbonyl group,
(d) a C₁₋₆ alkoxy group optionally substituted by a C₁₋₆ alkoxy group, and
(e) an aromatic heterocyclic group (e.g., thienyl),

(iii) a non-aromatic heterocyclic group (the non-aromatic heterocyclic group may be oxidized; e.g., tetrahydrofuryl, tetrahydropyranyl) optionally substituted by a hydroxy group, and

(iv) a C₁₋₆ alkoxy-carbonyl group optionally substituted by a C₆₋₁₄ aryl group (e.g., phenyl);
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom, a hydroxy group, a carbamoyl group and an aromatic heterocyclic group (e.g., furyl);
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl, tetrahydropyranyl) optionally substituted by 1 to 3 substituents selected from

(i) a C₁₋₆ alkyl group optionally substituted by 1 to 3 substituents selected from
(a) a hydroxy group,
(b) a C₆₋₁₄ aryl group (e.g., phenyl),
(c) a C₁₋₆ alkoxy group, and
(d) a non-aromatic heterocyclic group (the non-aromatic heterocyclic group may be oxidized; e.g., tetrahydrofuryl) optionally substituted by a C₁₋₆ alkyl group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group,

(vi) a carbamoyl group optionally mono- or di-substituted by a C₁₋₆ alkyl group optionally substituted by 1 to 3 substituents selected from a hydroxy group and a carbamoyl group,

(vii) a hydroxy group,

(viii) a C₆₋₁₄ aryl-carbonyl group (e.g., benzoyl),

(ix) a C₁₋₆ alkylsulfonyl group, and

(x) a C₆₋₁₄ arylsulfonyl group (e.g., phenylsulfonyl);
(11) a non-aromatic heterocyclyloxy group (the non-aromatic heterocycle may be oxidized; e.g., 1,1-dioxidotetrahydrothiopyranyloxy);
(12) a C₁₋₆ alkoxy-carbonyl group optionally substituted by a C₆₋₁₄ aryl group (e.g., phenyl);
(13) a carboxyl group;
(14) a non-aromatic heterocyclylcarbonyl group (the non-aromatic heterocycle may be oxidized; e.g., morpholinylcarbonyl, piperazinylcarbonyl) optionally substituted by a C₁₋₆ alkyl group optionally substituted by a C₆₋₁₄ aryl group (e.g., phenyl);
(15) a C₁₋₄ alkylenedioxy group optionally substituted by a halogen atom;
(16) a C₆₋₁₄ aryl group (e.g.,phenyl) optionally substituted by 1 to 3 substituents selected from a C₁₋₆ alkoxy group and a C₁₋₆ alkylsulfonyl group;
(17) an aromatic heterocyclic group (e.g., thienyl, pyridyl, tetrazolyl);
(18) a C₁₋₆ alkyl-carbonyl group optionally substituted by a hydroxy group;
(19) a C₆₋₁₄ aryl-carbonyl group (e.g., benzoyl);
(20) an oxo group;
(21) a C₁₋₆ alkylsulfonyl group optionally substituted by 1 to 3 halogen atoms;
(22) a C₆₋₁₄ arylsulfonyl group (e.g., phenylsulfonyl) optionally substituted by a C₁₋₆ alkoxy group;
(23) a C₃₋₁₀ cycloalkylsulfonyl group (e.g., cyclopropylsulfonyl);
(24) an aromatic heterocyclylsulfonyl group (e.g., pyridylsulfonyl, pyrazolylsulfonyl, thienylsulfonyl, furylsulfonyl, imidazolylsulfonyl) optionally substituted by 1 to 3 substituents selected from

(i) a C₁₋₆ alkyl group,

(ii) a C₁₋₆ alkoxy group,

(iii) a C₁₋₆ alkoxy-carbonyl group, and

(iv) a halogen atom; and
(25) a C₁₋₆ alkylthio group (e.g., methylthio)]
[further more preferably, Ra is
(A) a C₆₋₁₄ aryl group (e.g., phenyl) optionally substituted by 1 to 3 substituents selected from
(1) a halogen atom;

(2) a C₁₋₆ alkoxy group optionally substituted by 1 to 3 substituents selected from

(i) a hydroxy group,

(ii) a C₁₋₆ alkoxy group,

(iii) a C₆₋₁₄ aryl group (e.g., phenyl),

(iv) a C₁₋₆ alkylsulfonyl group, and

(v) a non-aromatic heterocyclic group (the non-aromatic heterocyclic group may be oxidized; e.g., 1,1-dioxidothiomorpholinyl, imidazolidinyl) optionally substituted by an oxo group;
(3) a C₁₋₆ alkyl group optionally substituted by 1 to 3 substituents selected from

(i) an amino group,

(ii) a C₁₋₆ alkoxy-carbonyl group,

(iii) a carboxyl group,

(iv) a carbamoyl group optionally mono- or di-substituted by a C₁₋₆ alkyl group optionally substituted by a hydroxy group, and

(v) a non-aromatic heterocyclylcarbonyl group (the non-aromatic heterocycle may be oxidized; e.g., morpholinylcarbonyl);
(4) an amino group optionally mono- or di-substituted by substituent(s) selected from

(i) a C₁₋₁₀ alkyl group optionally substituted by 1 to 3 substituents selected from
(a) a hydroxy group,
(b) a C₁₋₆ alkoxy group optionally substituted by a C₆₋₁₄ aryl group (e.g., phenyl),
(c) a carboxyl group,
(d) a C₃₋₁₀ cycloalkyl group (e.g., cyclopropyl) optionally substituted by a C₁₋₆ alkoxy-carbonyl group,
(e) a halogen atom,
(f) an aromatic heterocyclic group (e.g., furyl, pyridyl, indolyl, imidazolyl, thienyl, pyrazolyl, pyrrolyl) optionally substituted by 1 to 3 substituents selected from
   1) a C₁₋₆ alkyl group optionally substituted by a hydroxy group,
   2) a C₁₋₆ alkoxy-carbonyl group,
   3) a carboxyl group,
   4) a halogen atom, and
   5) a C₁₋₆ alkylthio group,
(g) a C₆₋₁₄ aryl group (e.g., phenyl) optionally substituted by 1 to 3 substituents selected from
   1) an amino group optionally mono- or di-substituted by substituent(s) selected from a C₁₋₆ alkyl group and a C₁₋₆ alkyl-carbonyl group,
   2) a C₁₋₄ alkylenedioxy group,
   3) a hydroxy group, and
   4) a C₁₋₆ alkoxy group optionally substituted by a carboxyl group,
(h) a C₁₋₆ alkylthio group, and

(i) an amino group optionally mono- or di-substituted by a C₁₋₆ alkoxy-carbonyl group optionally substituted by a C₆₋₁₄ aryl group (e.g., phenyl),

(ii) a C₁₋₆ alkyl-carbonyl group optionally substituted by 1 to 3 substituents selected from
(a) a carboxyl group,
(b) a C₆₋₁₄ aryl group (e.g., phenyl), and
(c) an amino group optionally mono- or di-substituted by a C₁₋₆ alkyl-carbonyl group, and

(iii) a non-aromatic heterocyclic group (the non-aromatic heterocyclic group may be oxidized; e.g., tetrahydrofuryl) optionally substituted by a hydroxy group;
(5) a nitro group;
(6) a hydroxy group;
(7) a cyano group;
(8) a carbamoyl group optionally mono- or di-substituted by a C₁₋₆ alkyl group optionally substituted by 1 to 3 substituents selected from a halogen atom and a hydroxy group;
(9) a C₆₋₁₄ aryloxy group (e.g., phenoxy) optionally substituted by 1 to 3 halogen atoms;
(10) a non-aromatic heterocyclic group (the non-aromatic heterocyclic group may be oxidized; e.g., morpholinyl, thiomorpholinyl, 1-oxidothiomorpholinyl, 1,1-dioxidothiomorpholinyl, piperidinyl, piperazinyl) optionally substituted by 1 to 3 substituents selected from

(i) a C₁₋₆ alkyl group optionally substituted by a hydroxy group,

(ii) a C₁₋₆ alkyl-carbonyl group optionally substituted by an amino group optionally mono- or di-substituted by a C₁₋₆ alkyl-carbonyl group,

(iii) a C₁₋₆ alkoxy-carbonyl group,

(iv) a carboxyl group,

(v) an oxo group, and

(vi) a carbamoyl group optionally mono- or di-substituted by a C₁₋₆ alkyl group optionally substituted by 1 to 3 substituents selected from a hydroxy group and a carbamoyl group;
(11) a non-aromatic heterocyclyloxy group (the non-aromatic heterocycle maybe oxidized; e.g., 1,1-dioxidotetrahydrohiopyranyloxy);
(12) a C₁₋₆ alkoxy-carbonyl group;
(13) a carboxyl group;
(14) a non-aromatic heterocyclylcarbonyl group (the non-aromatic heterocycle may be oxidized; e.g., morpholinylcarbonyl);
(15) a C₁₋₄ alkylenedioxy group optionally substituted by a halogen atom;
(16) an aromatic heterocyclic group (e.g., tetrazolyl); and
(17) a C₁₋₆ alkylsulfonyl group;
(B) a 5 or 6-membered aromatic heterocyclic group (e.g., pyridyl, thienyl);
(C) a 5 or 6-membered non-aromatic heterocyclic group (the non-aromatic heterocyclic group may be oxidized; e.g., pyrrolidinyl, piperidinyl, hexamethyleneiminyl, tetrahydrofuryl, tetrahydropyranyl) optionally substituted by 1 to 3 substituents selected from
(1) a C₁₋₆ alkyl group optionally substituted by 1 to 3 substituents selected from

(i) a C₆₋₁₄ aryl group (e.g., phenyl) optionally substituted by a C₁₋₆ alkylsulfonyl group,

(ii) a C₃-₁0 cycloalkyl group (e.g., cyclopropyl),

(iii) an aromatic heterocyclic group (e.g., furyl) optionally substituted by 1 to 3 substituents selected from a carboxyl group and a C₁₋₆ alkoxy-carbonyl group,

(iv) a non-aromatic heterocyclic group (the non-aromatic heterocyclic group may be oxidized; e.g., tetrahydrofuryl) optionally substituted by a C₁₋₆ alkyl group, and

(v) a C₁₋₆ alkoxy group;
(2) a C₁₋₆ alkyl-carbonyl group optionally substituted by a hydroxy group;
(3) a C₁₋₆ alkoxy-carbonyl group optionally substituted by a C₆₋₁₄ aryl group (e.g., phenyl);
(4) a C₆₋₁₄ aryl-carbonyl group (e.g., benzoyl);
(5) an oxo group;
(6) a hydroxy group;
(7) a C₁₋₆ alkylsulfonyl group optionally substituted by 1 to 3 halogen atoms;
(8) a C₆₋₁₄ arylsulfonyl group (e.g., phenylsulfonyl) optionally substituted by a C₁₋₆ alkoxy group;
(9) a C₃₋₁₀ cycloalkylsulfonyl group (e.g., cyclopropylsulfonyl);
(10) an aromatic heterocyclylsulfonyl group (e.g., pyridylsulfonyl, pyrazolylsulfonyl, thienylsulfonyl, furylsulfonyl, imidazolylsulfonyl) optionally substituted by 1 to 3 substituents selected from

(i) a C₁₋₆ alkyl group,

(ii) a C₁₋₆ alkoxy group,

(iii) a C₁₋₆ alkoxy-carbonyl group, and

(iv) a halogen atom;
(11) a C₆₋₁₄ aryl group (e.g., phenyl) optionally substituted by a C₁₋₆ alkylsulfonyl group; and
(12) an aromatic heterocyclic group (e.g., pyridyl, thienyl);
(D) a C₃₋₁₀ cycloalkyl group condensed with a benzene ring (e.g., indanyl, tetrahydronaphthyl); or
(E) a C₁₋₆ alkyl group optionally substituted by 1 to 3 substituents selected from
(1) a hydroxy group;
(2) a C₁₋₆ alkoxy group;
(3) a C₁₋₆ alkylthio group (e.g., methylthio);
(4) a C₆₋₁₄ aryl group (e.g., phenyl) optionally substituted by a C₁₋₆ alkoxy group;
(5) an aromatic heterocyclic group (e.g., thienyl, pyridyl);
(6) a non-aromatic heterocyclic group (the non-aromatic heterocyclic group may be oxidized; e.g., piperidinyl, tetrahydropyranyl) optionally substituted by 1 to 3 substituents selected from

(i) a hydroxy group,

(ii) a C₁₋₆ alkyl group optionally substituted by 1 to 3 substituents selected from
(a) a C₆₋₁₄ aryl group (e.g., phenyl),
(b) a alkoxy group, and
(c) a non-aromatic heterocyclic group (the non-aromatic heterocyclic group may be oxidized; e.g., tetrahydrofuryl) optionally substituted by a C₁₋₆ alkyl group,

(iii) a C₁₋₆ alkyl-carbonyl group,

(iv) a C₆₋₁₄ aryl-carbonyl group (e.g., benzoyl),

(v) a C₁₋₆ alkylsulfonyl group, and

(vi) a C₆₋₁₄ arylsulfonyl group (e.g., phenylsulfonyl);
(7) a non-aromatic heterocyclylcarbonyl group (the non-aromatic heterocycle may be oxidized; e.g., morpholinylcarbonyl, piperazinylcarbonyl) optionally substituted by C₁₋₆ alkyl group optionally substituted by a C₆₋₁₄ aryl group (e.g., phenyl);
(8) an amino group optionally mono- or di-substituted by substituent(s) selected from

(i) a C₁₋₆ alkoxy-carbonyl group optionally substituted by a C₆₋₁₄ aryl group (e.g., phenyl),

(ii) a non-aromatic heterocyclic group (the non-aromatic heterocyclic group may be oxidized; e.g., tetrahydropyranyl), and

(iii) a C₁₋₆ alkyl-carbonyl group optionally substituted by 1 to 3 substituents selected from
(a) an amino group optionally mono- or di-substituted by a C₁₋₆ alkyl-carbonyl group,
(b) a C₁₋₆ alkoxy group optionally substituted by a C₁₋₆ alkoxy group, and
(c) an aromatic heterocyclic group (e.g., thienyl); and
(9) a carbamoyl group optionally mono- or di-substituted by a C₁₋₆ alkyl group optionally substituted by 1 to 3 substituents selected from a hydroxy group, a carbamoyl group and an aromatic heterocyclic group (e.g., furyl); and
Rb is
(A) a C₆₋₁₄ aryl group (e.g., phenyl) optionally substituted by 1 to 3 substituents selected from
(1) a halogen atom;
(2) a hydroxy group; and
(3) a C₁₋₆ alkoxy group optionally substituted by 1 to 3 substituents selected from

(i) a C₆₋₁₄ aryl group (e.g., phenyl),

(ii) a carboxyl group,

(iii) a C₁₋₆ alkoxy-carbonyl group,

(iv) a carbamoyl group, and

(v) a C₁₋₆ alkoxy group;
(B) a 5 or 6-membered aromatic heterocyclic group (e.g., pyridyl, thiazolyl, thienyl);
(C) a C₁₋₆ alkyl group (e.g., methyl, propyl); or
(D) a C₃₋₁₀ cycloalkyl group (e.g., cyclopropyl, cyclohexyl)];
X is a bond or a straight chain C₁₋₆ alkylene group optionally having substituent(s) (preferably a bond);
Y is —CO—, —CH₂—, —CH₂CO— or —SO₂— (preferably —CO— or —CH₂—, more preferably —CO—);
Rc is
(1) an optionally substituted C₁₋₆ alkyl group;
(2) an optionally substituted C₆₋₁₄ aryl group;
(3) an optionally substituted C₂₋₆ alkenyl group;
(4) an optionally substituted C₁₋₆ alkyl-carbonyl group; or
(5) an optionally substituted carbamoyl group;
[preferably, Rc is a C₁₋₆ alkyl group optionally substituted by 1 to 3 substituents selected from

(i) an optionally substituted C₆₋₁₄ aryl group, and

(ii) an optionally substituted C₁₋₆ alkoxy group]
[specifically, Rc is
(1) a C₁₋₆ alkyl group optionally substituted by 1 to 3 substituents selected from

(i) a hydroxy group,

(ii) a C₆₋₁₄ aryl group (e.g., phenyl) optionally substituted by 1 to 3 substituents selected from
(a) a carboxyl group,
(b) a hydroxy group,
(c) a C₁₋₆ alkyl group optionally substituted by 1 to 3 substituents selected from
   (A) a hydroxy group, and
   (B) a halogen atom,
(d) a C₁₋₆ alkoxy group optionally substituted by 1 to 3 substituents selected from
   (A) a C₁₋₆ alkoxy group,
   (B) a carbamoyl group optionally mono- or di-substituted by substituent(s) selected from a alkyl group optionally substituted by a carbamoyl group, and a C₁₋₆ alkylsulfonyl group,
   (C) a carboxyl group,
   (D) a C₁₋₆ alkoxy-carbonyl group optionally substituted by a non-aromatic heterocyclic group (e.g., dioxolyl) optionally substituted by 1 to 3 substituents selected from an oxo group and a C₁₋₆ alkyl group,
   (E) a cyano group, and
   (F) a non-aromatic heterocyclic group (e.g., oxadiazolinyl) optionally substituted by an oxo group,
(e) a carbamoyl group optionally mono- or di-substituted by substituent(s) selected from
   (A) a C₁₋₆ alkyl group optionally substituted by a hydroxy group, and
   (B) a C₁₋₆ alkylsulfonyl group,
(f) a non-aromatic heterocyclic group (e.g., oxadiazolinyl) optionally substituted by an oxo group,
(g) an aromatic heterocyclic group (e.g., tetrazolyl),
(h) a C₁₋₆ alkoxy-carbonyl group optionally substituted by a non-aromatic heterocyclic group (e.g., dioxolyl) optionally substituted by 1 to 3 substituents selected from an oxo group and a C₁₋₆ alkyl group,
(i) a cyano group,
(j) a sulfamoyl group, and
(k) a halogen atom,

(iii) a C₆₋₁₄ aryloxy group (e.g., phenoxy) optionally substituted by 1 to 3 substituents selected from
(a) a carboxyl group,
(b) a carbamoyl group,
(c) a C₁₋₆ alkyl group optionally substituted by 1 to 3 substituents selected from a carboxyl group and a halogen atom,
(d) a C₁₋₄ alkylenedioxy group,
(e) a C₁₋₆ alkyl-carbonyl group, and
(f) a cyano group,

(iv) a C₃₋₁₀ cycloalkyl group (e.g., cyclopropyl, cyclohexyl),

(v) an aromatic heterocyclic group (e.g., imidazolyl, thienyl, pyridyl, oxazolyl, oxadiazolyl, benzimidazolyl) optionally substituted by 1 to 3 substituents selected from
(a) a C₆₋₁₄ aryl group (e.g., phenyl), and
(b) a C₁₋₆ alkyl group,

(vi) a non-aromatic heterocyclic group (e.g., morpholinyl, piperidinyl, oxazolidinyl) optionally substituted by 1 to 3 substituents selected from
(a) a carboxy group,
(b) a C₁₋₆ alkoxy-carbonyl group,
(c) a carbamoyl group optionally mono- or di-substituted by a C₁₋₆ alkyl group optionally substituted by 1 to 3 substituents selected from a hydroxy group and a carbamoyl group, and
(d) an oxo group,

(vii) a C₁₋₆ alkoxy group optionally substituted by a C₆₋₁₄ aryl group (e.g., phenyl) optionally substituted by a C₁₋₆ alkylsulfonyl group,

(viii) a C₁₋₆ alkylthio group,

(ix) a C₆₋₁₄ arylthio group (e.g., phenylthio),

(x) a C₆₋₁₄ arylsulfinyl group (e.g., phenylsulfinyl),

(xi) a C₆₋₁₄ arylsulfonyl group (e.g., phenylsulfonyl),

(xii) an amino group optionally mono- or di-substituted by substituent(s) selected from
(a) a C₁₋₆ alkyl group optionally substituted by 1 to 3 substituents selected from
   (A) a C₆₋₁₄ aryl group (e.g., phenyl), and
   (B) a carbamoyl group,
(b) a C₆₋₁₄ aryl group (e.g., phenyl),
(c) a C₁₋₆ alkyl-carbonyl group optionally substituted by 1 to 3 substituents selected from
   (A) a carboxyl group,
   (B) a C₁₋₆ alkoxy-carbonyl group,
   (C) a carbamoyl group optionally mono- or di-substituted by a C₃₋₁₀ cycloalkyl group, and
   (D) a non-aromatic heterocyclylcarbonyl group (e.g., morpholinylcarbonyl),
(d) a carbamoyl group optionally mono- or di-substituted by a C₁₋₆ alkyl group optionally substituted by 1 to 3 substituents selected from
   (A) a carboxyl group,
   (B) a C₁₋₆ alkoxy-carbonyl group, and
   (C) a carbamoyl group,
(e) a C₆₋₁₄ aryl-carbonyl group optionally substituted by a C₁₋₆ alkoxy group, and
(f) a C₃₋₁₀ cycloalkyl-carbonyl group,

(xiii) a cyano group,

(xiv) a carboxyl group, and

(xv) a carbamoyl group optionally mono- or di-substituted by substituent(s) selected from a C₁₋₆ alkyl group and a C₆₋₁₄ aryl group (e.g., phenyl);
(2) a C₆₋₁₄ aryl group (e.g., phenyl);
(3) a C₂₋₆ alkenyl group optionally substituted by a C₆₋₁₄ aryl group (e.g., phenyl);
(4) a C₁₋₆ alkyl-carbonyl group;
(5) a carbamoyl group optionally mono- or di-substituted by a C₁₋₆ alkyl optionally substituted by a C₆₋₁₄ aryl group (e.g., phenyl); or
(6) a carbamoyl group optionally mono- or di-substituted by an aromatic heterocyclic group (e.g., pyridyl);
more preferably Rc is a C₁₋₆ alkyl group optionally substituted by 1 to 3 substituents selected from

(i) a C₆₋₁₄ aryl group (e.g., phenyl) optionally substituted by a carboxyl group, and

(ii) a C₁₋₆ alkoxy group optionally substituted by a C₆₋₁₄ aryl group (e.g., phenyl) optionally substituted by a C₁₋₆ alkylsulfonyl group];
m and n are each independently 1 or 2 (preferably 1, more preferably both m and n are 1); and
ring B is optionally further substituted by a C₁₋₆ alkyl group optionally substituted by a hydroxy group.

### [Compound C]

A compound selected from the group consisting of
(2R)-2-benzyl-1-{[1-(2,3-dimethoxyphenyl)-5-methyl-2-phenyl-1H-pyrrol-3-yl]carbonyl}piperazine (Example 43),
4-(3-(4-{[(2R)-2-benzylpiperazin-1-yl]carbonyl}-5-phenyl-1H-imidazol-1-yl)phenyl]morpholine (Example 56),
(2R)-2-benzyl-1-([1-(2,3-dimethoxyphenyl)-5-phenyl-1H-imidazol-4-yl]carbonyl}piperazine (Example 57),
(2R)-2-benzyl-1-{[1-(2,3-dihydro-1H-inden-2-yl)-5-phenyl-1H-imidazol-4-yl]carbonyl}piperazine (Example 73),
2-(3-[(2-benzylpiperazin-1-yl)carbonyl]-2-phenyl-1H-pyrrol-1-yl}-N-butylaniline (Example 99),
4-(3-(3-{[(2R)-2-benzylpiperazin-1-yl]carbonyl}-5-methyl-2-phenyl-1H-pyrrol-1-yl)phenyl]morpholine (Example 105),
4-[((2R)-1-{[1-(3-morpholinophenyl)-5-phenyl-1H-imidazol-4-yl]carbonyl}piperazin-2-yl)methyl]benzoic acid (Example 161),
4-[3-(4-{[(2S)-2-({[4-(methylsulfonyl)benzyl]oxy}methyl)piperazin-1-yl]carbonyl}-5-phenyl-1H-imidazol-1-yl)phenyl]morpholine (Example 472),
(2R)-2-benzyl-1-[(2-methoxy-1,5-diphenyl-1H-imidazol-4-yl)carbonyl]piperazine (Example 475),
(2R)-2-benzyl-1-({5-phenyl-1-[1-(phenylsulfonyl)piperidin-3-yl]-1H-imidazol-4-yl}carbonyl)piperazine (Example 476),
(2R)-2-benzyl-1-[(1-{1-[(6-methoxypyridin-3-yl)sulfonyl]piperidin-3-yl}-5-phenyl-1H-imidazol-4-yl)carbonyl]piperazine (Example 477), and
4-[(3S)-3-(4-{[(2R)-2-benzylpiperazin-1-yl]carbonyl}-5-phenyl-1H-imidazol-1-yl)-5-phenylpentanoyl]morpholine (Example 478)
   or a salt thereof.

As a salt of compound (I) or compound (I′), for example, metal salts, ammonium salts, salts with organic bases, salts with inorganic acids, salts with organic acids, salts with basic or acidic amino acids, and the like can be mentioned.

Preferable examples of the metal salt include alkali metal salts such as sodium salt, potassium salt and the like; alkaline earth metal salts such as calcium salt, magnesium salt, barium salt and the like; aluminum salt and the like.

Preferable examples of the salt with organic base include a salt with trimethylamine, triethylamine, pyridine, picoline, 2,6-lutidine, ethanolamine, diethanolamine, triethanolamine, cyclohexylamine, dicyclohexylamine, N,N-dibenzylethylenediamine or the like.

Preferable examples, of the salt with inorganic acid include a salt with hydrochloric acid, hydrobromic acid, nitric acid, sulfuric acid, phosphoric acid or the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid or the like.

Preferable examples of the salt with basic amino acid include a salt with arginine, lysine, ornithine or the like.

Preferable examples of the salt with acidic amino acid include a salt with aspartic acid, glutamic acid or the like.

Of these, a pharmaceutically acceptable salt is preferable. When the compound has an acidic functional group, for example, inorganic salts such as alkali metal salts (e.g., sodium salt, potassium salt, etc.), alkaline earth metal salts (e.g., calcium salt, magnesium salt, barium salt, etc.) and the like, ammonium salts, and the like can be mentioned. When the compound has a basic functional group, for example, salts with inorganic acids such as hydrochloric acid, hydrobromic acid, nitric acid, sulfuric acid, phosphoric acid and the like, and salts with organic acids such as acetic acid, phthalic acid, fumaric acid, oxalic acid, tartaric acid, maleic acid, citric acid, succinic acid, methanesulfonic acid, p-toluenesulfonic acid and the like, can be mentioned.

The production methods of compound (I) are shown in the following.

Compound (I) is obtained by, for example, a method shown in the following reaction scheme or a method analogous thereto, or the like.

Each of compounds (II)-(VIII) shown in the reaction scheme may form a salt, and as such salt, salts similar to the salts of compound (I) can be mentioned.

The compound obtained in each step can also be used for the next reaction directly as the reaction mixture or as a crude product. In addition, it can also be isolated from the reaction mixture according to a conventional method, and can be isolated and purified by a known method such as phase transfer, concentration, solvent extraction, fractional distillation, pH conversion, crystallization, recrystallization, chromatography and the like.

The schematic drawings of the reaction scheme are shown in the following. Each symbol of the compounds in the schematic drawings is as defined above. PG is a protecting group such as a benzyl group, a tert-butoxycarbonyl group and the like.

### (Reaction 1) Production Method of Compound (I) Wherein Y═CO

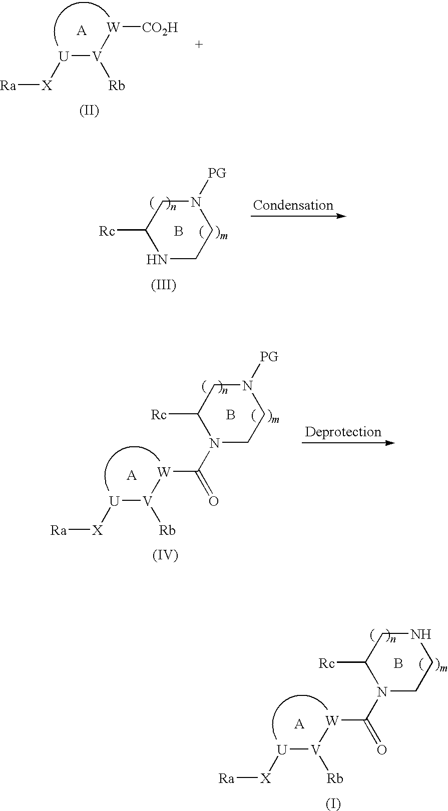

wherein each symbol is as defined above.

In the scheme, compound (II) can be produced according to a method known per se, for example, in the case where ring A is an imidazole ring, the method disclosed in Journal of Organic Chemistry, Vol. 59, pp. 7635-7642 (1994) or the like, or a method analogous thereto. Compound (III) can be produced according to a method known per se, for example, the method disclosed in WO 2003/000181 or the like, or a method analogous thereto. When compound (II) or compound (III) is commercially available, the commercial product may be also used directly.

Compound (IV) can be produced by a condensation reaction of compound (II) and compound (III).

The condensation reaction is carried out by a conventional peptide synthesis technique, for example, an acid chloride method, an acid anhydride method, a mixed acid anhydride method, a method of using N,N′-dicyclohexylcarbodiimide (DCC), an active ester method, a method of using N,N′-carbonyldiimidazole (CDI), a method of using diethyl cyanophosphate (DEPC), a method of using N-ethyl-N′-(3-dimethylaminopropyl)carbodiimide hydrochloride (WSC.HCl) and 1-hydroxybenzotriazole (HOBt), or the like.

Compound (III) is used in an amount of about 1.0 to 2.0 mol, preferably about 1.0 to 1.1 mol, per 1 mol of compound (II).

The reagent used for the aforementioned methods is used in an amount of about 1.0 to 2.0 mol, preferably about 1.1 to 1.3 mol, per 1 mol of compound (II).

The condensation reaction is preferably carried out in a solvent, and as the solvent to be used, halogenated hydrocarbons such as dichloromethane, chloroform, carbon tetrachloride, 1,2-dichloroethane and the like; ethers such as diethyl ether, tetrahydrofuran, dioxane and the like; amides such as N,N-dimethylformamide, N,N-dimethylacetamide and the like; dimethylsulfoxide, pyridine, acetonitrile, or a solvent mixture thereof can be mentioned.

The reaction temperature is usually −10 to 80° C., preferably 0 to 30° C.

The reaction time may vary depending on the reagent or solvent to be used, but is usually 30 minutes to 3 days, preferably 30 minutes to 15 hours.

Compound (IV) can be also produced by further carrying out one or a plurality of known hydrolysis reaction, acylation reaction, alkylation reaction, amination reaction, oxidation-reduction reaction, cyclization reaction, carbon chain extension reaction, substituent exchange reaction and the like, in combination with the aforementioned reaction, as desired.

### (Reaction 2) Production Method of Compound (I) Wherein Y═CH₂CO

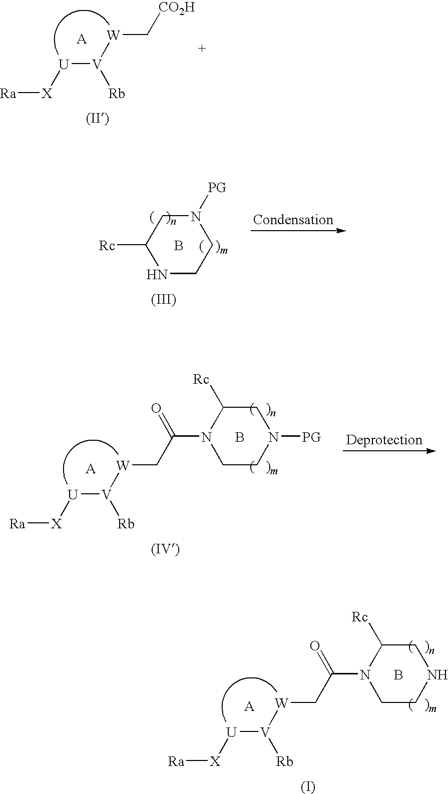

wherein each symbol is as defined above.

Compound (II′) can be produced according to a method known per se, for example, in the case where ring A is a pyrazole ring, the method disclosed in Journal of Heterocyclic Chemistry, Vol. 30, pp. 997-1002 (1993) or the like, or a method analogous thereto. When compound (II′) is commercially available, the commercial product can be also used directly.

Compound (IV′) can be produced by a condensation reaction of compound (II′) and compound (III), according to the method similar to the method as shown in the aforementioned production method (Reaction 1).

Compound (IV′) can be also produced by further carrying out one or a plurality of known hydrolysis reaction, acylation reaction, alkylation reaction, amination reaction, oxidation-reduction reaction, cyclization reaction, carbon chain extension reaction, substituent exchange reaction and the like, in combination with the aforementioned reaction, as desired.

### (Reaction 3) Production Method of Compound (I) Wherein Y═CH₂

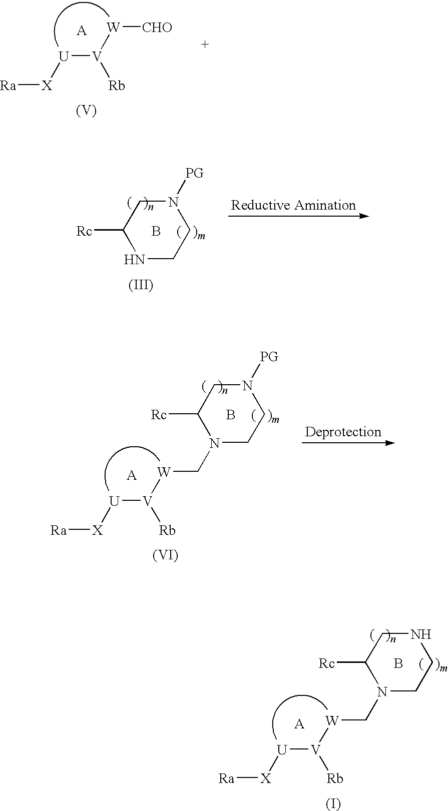

wherein each symbol is as defined above.

Compound (V) can be produced according to a method known per se, for example, in the case where ring A is a pyrazole ring, the method disclosed in Journal of Heterocyclic Chemistry, Vol. 34, pp. 963-968 (1997) or the like, or a method analogous thereto. When compound (V) is commercially available, the commercial product can be also used directly.

Compound (VI) can be produced by a reductive amination reaction of compound (V) and compound (III).

Compound (III) is used in an amount of about 1.0 to 2.0 mol, preferably about 1.0 to 1.1 mol, per 1 mol of compound (V).

The reducing agent is used in an amount of about 1.0 to 3.0 mol, preferably about 1.1 to 1.5 mol, per 1 mol of compound (V).

As the reducing agent, metal hydrogen complexes such as sodium borohydride, lithium aluminum hydride, sodium triacetoxyborohydride, sodium cyanoborohydride and the like can be used.

This reaction is advantageously carried out by adding about 0.5 to 3.0 mol, preferably about 1.0 to 1.2 mol of an organic acid (e.g., acetic acid, benzoic acid, etc.), in addition to the aforementioned reducing agent.

This reaction is preferably carried out in a solvent, and as the solvent to be used, the aforementioned halogenated hydrocarbons, ethers, amides or solvent mixtures thereof can be mentioned.

The reaction temperature is usually −10 to 80° C., preferably 0 to 30° C.

The reaction time may vary depending on the reagent or solvent to be used, but is usually 30 minutes to 3 days, preferably 30 minutes to 15 hours.

Compound (VI) can be also produced by further carrying out one or a plurality of known hydrolysis reaction, acylation reaction, alkylation reaction, amination reaction, oxidation-reduction reaction, cyclization reaction, carbon chain extension reaction, substituent exchange reaction and the like, in combination with the aforementioned reaction, as desired.

### (Reaction 4) Production Method of Compound (I) Wherein Y═SO₂

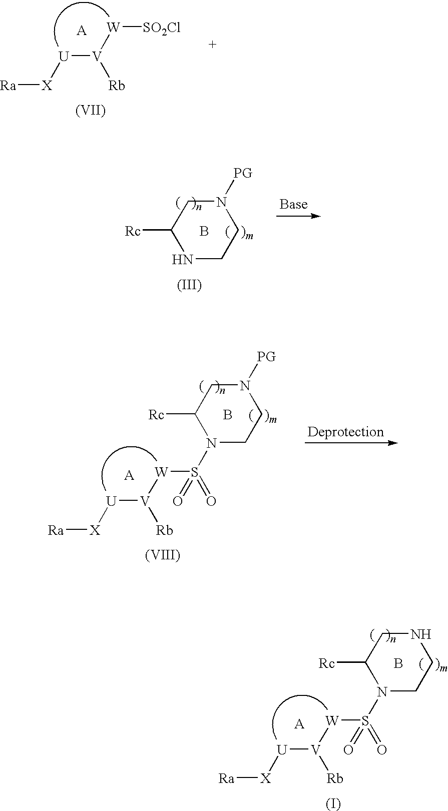

wherein each symbol is as defined above.

Compound (VII) can be produced according to a method known per se, for example, in the case where ring A is a pyrazole ring, the method disclosed in Journal of Chemical Society, (C), pp. 78-81 (1970) or the like, or a method analogous thereto. When compound (VII) is commercially available, the commercial product can be also used directly.

Compound (VIII) can be produced by reacting compound (VII) with compound (III) in the presence of base.

Compound (III) is used in an amount of about 1.0 to 2.0 mol, preferably about 1.0 to 1.1 mol, per 1 mol of compound (VII).

The base is used in an amount of about 1.0 to 3.0 mol, preferably about 1.1 to 1.5 mol, per 1 mol of compound (VII).

As the base, for example, inorganic bases such as sodium hydroxide, potassium hydroxide and the like; basic salts such as sodium carbonate, potassium carbonate, cesium carbonate, sodium hydrogencarbonate and the like; aromatic amines such as pyridine, lutidine and the like; tertiary amines such as triethylamine, N,N-diisopropylethylamine, tripropylamine, tributylamine, cyclohexyldimethylamine, 4-(dimethylamino)pyridine, N,N-dimethylaniline, N-methylpiperidine, N-methylpyrrolidine, N-methylmorpholine and the like; and the like can be used.

This reaction is preferably carried out in a solvent, and as the solvent to be used, the aforementioned halogenated hydrocarbons, ethers, amides, dimethylsulfoxide, pyridine, acetonitrile, water or solvent mixtures thereof can be mentioned.

The reaction temperature is usually −10 to 80° C., preferably 0 to 30° C.

The reaction time may vary depending on the reagent or solvent to be used, but is usually 30 minutes to 3 days, preferably 30 minutes to 15 hours.

Compound (VIII) can also be produced by further carrying out one or a plurality of known hydrolysis reaction, acylation reaction, alkylation reaction, amination reaction, oxidation-reduction reaction, cyclization reaction, carbon chain extension reaction, substituent exchange reaction and the like, in combination with the aforementioned reaction, as desired.

In the aforementioned production methods (Reaction 1 to Reaction 4), compound (I) can be produced by removing the protecting group PG of compound (IV), compound (IV′), compound (VI) or compound (VIII).

In addition, in each of the aforementioned reactions, when the starting compound has an amino group, a carboxyl group or a hydroxy group as a substituent, a protecting group generally used in peptide chemistry and the like may be introduced into these groups. By removing the protecting group as necessary after the reaction, the objective compound can be obtained. Introduction or removal of these protective groups may be carried out according to a method known per se, for example, the method disclosed in Theodora W. Greene and Peter G. M. Wuts, “Protective Groups in Organic Synthesis, 3^{rd} Ed.”, Wiley-Interscience (1999); or the like.

As the amino-protecting group, for example, formyl group; C₁₋₆ alkyl-carbonyl group, phenylcarbonyl group, C₁₋₆ alkoxy-carbonyl group, allyloxycarbonyl group (Alloc), phenyloxycarbonyl group, fluorenylmethyloxycarbonyl group (Fmoc), C₇₋₁₀ aralkyl-carbonyl group (e.g., benzylcarbonyl and the like), C₇₋₁₀ aralkyloxy-carbonyl group (e.g., benzyloxycarbonyl (Z) and the like), C₇₋₁₀ aralkyl group (e.g., benzyl and the like), trityl group, phthaloyl group, dithiasuccinoyl group, N,N-dimethylaminomethylene group, each optionally having substituent(s), and the like can be mentioned. As the substituent(s), for example, phenyl group, halogen atom, C₁₋₆ alkyl-carbonyl group, C₁₋₆ alkoxy group optionally substituted by halogen atom(s) (e.g., methoxy, ethoxy, trifluoromethoxy and the like), nitro group and the like can be used. The number of the substituent(s) is 1 to 3.

As the carboxyl-protecting group, for example, C₁₋₆ alkyl group, allyl group, benzyl group, phenyl group, trityl group, trialkylsilyl group, each optionally having substituent(s), and the like can be mentioned. As the substituent(s), for example, halogen atom, formyl group, C₁₋₆ alkyl-carbonyl group, C₁₋₆ alkoxy group optionally substituted by halogen atom(s) (e.g., methoxy, ethoxy, trifluoromethoxy and the like), nitro group and the like can be used. The number of the substituent(s) is 1 to 3.

As the hydroxy-protecting group, for example, C₁₋₆ alkyl group, C₇₋₂₀ aralkyl group (e.g., benzyl, trityl and the like), formyl group, C₁₋₆ alkyl-carbonyl group, benzoyl group, C₇₋₁₀ aralkyl-carbonyl group (e.g., benzylcarbonyl and the like), 2-tetrahydropyranyl group, tetrahydrofuranyl group, trialkylsilyl group (e.g., trimethylsilyl, tert-butyldimethylsilyl, diisopropylethylsilyl and the like), each optionally having substituent(s), and the like can be mentioned. As the substituent(s), for example, halogen atom, C₁₋₆ alkyl group, phenyl group, C₇₋₁₀ aralkyl group (e.g., benzyl and the like), C₁₋₆ alkoxy group, nitro group and the like can be used. The number of the substituent(s) is 1 to 4.

Compound (I′) can be also produced according to the method similar to the method as shown in the aforementioned production methods.

When compound (I) or compound (I′) is obtained as a free compound, it can be converted to the object salt according to a method known per se or a method analogous thereto, and when it is obtained as a salt, it can be converted to a free compound or the object salt according to a method known per se or a method analogous thereto.

Compound (I) and compound (I′) (hereinafter, in the case of referring to compound (I), the compound includes compound (I′) as well) may be used as a prodrug. A prodrug of compound (I) means a compound which is converted to compound (I) with a reaction due to an enzyme, an gastric acid, etc. under the physiological condition in the living body, that is, a compound which is converted to compound (I) with oxidation, reduction, hydrolysis, etc. according to an enzyme; a compound which is converted to compound (I) by hydrolysis etc. due to gastric acid, etc.

Examples of a prodrug of compound (I) include a compound wherein an amino group of compound (I) is acylated, alkylated or phosphorylated (e.g., compound wherein amino group of compound (I) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy group of compound (I) is acylated, alkylated, phosphorylated or borated (e.g., a compound wherein a hydroxy group of compound (I) is acetylated, palmitoylated, propanoylated, pivaloylated, succinylated, fumarylated, alanylated or dimethylaminomethylcarbonylated, and the like); a compound wherein a carboxyl group of compound (I) is esterified or amidated (e.g., a compound wherein a carboxyl group of compound (I) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated, and the like) and the like. These compounds can be produced from compound (I) by a method known per se.

A prodrug of compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

When compound (I) has an isomer such as optical isomer, steric isomer, positional isomer, rotational isomer and the like, any isomers and a mixture thereof are encompassed in compound (I). For example, when compound (I) has an optical isomer, an optical isomer resolved from a racemate is also encompassed in compound (I). Such isomer can be obtained as a single product by a synthesis method or a separation method (concentration, solvent extraction, column chromatography, recrystallization etc.) known per se.

Compound (I) may be a crystal, and both a single crystal and crystal mixtures are encompassed in compound (I). Crystals can be produced by crystallization according to crystallization methods known per se.

Compound (I) may be a solvate (e.g., hydrate etc.) or a non-solvate, both of which are encompassed in compound (I).

A compound labeled with an isotope (e.g., ³H, ¹⁴C, ³⁵S, ¹²⁵I and the like) and the like is also encompassed in compound (I).

Compound (I) or its prodrug, or salts thereof (hereinafter, sometimes to be abbreviated to as a compound of the present invention) exhibit excellent renin inhibitory activity. They have low toxicity (e.g., acute toxicity, chronic toxicity, genetic toxicity, reproductive toxicity, cardiac toxicity, drug interaction, carcinogenicity, etc.) and high water-solubility, and are excellent in the aspects of stability, pharmacokinetics (absorbability, distribution, metabolism, excretion, etc.) and efficacy, thus being useful as medicine.

The compound of the present invention acts as a renin inhibitory drug in mammals (e.g., mouse, rat, hamster, rabbit, cat, dog, cattle, sheep, monkey, human, etc.), and is useful as a drug inhibiting the RA system by inhibiting the biosynthesis of AII, and is useful as an agent for the prophylaxis or treatment of various diseases caused by the RA system.

As such diseases, for example, blood pressure circadian rhythm abnormality, heart diseases (e.g., cardiac hypertrophy, acute heart failure and chronic heart failure including congestive heart failure, failure of expansion, cardiac myopathy, angina pectoris, myocarditis, atrial fibrillation, arrhythmia, tachycardia, cardiac infraction etc.), cerebrovascular disorders (e.g., asymptomatic cerebrovascular disorder, transient cerebral ischemia, apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after cardiac infarction, renal diseases (e.g., nephritis, glomerulonephritis, glomerulosclerosis, renal failure, nephrotic syndrome, thrombotic vasculopathy, complication of dialysis, organ damage including nephropathy by radiation irradiation etc.), arteriosclerosis including atherosclerosis (e.g., aneurysm, coronary arteriosclerosis, cerebral arteriosclerosis, peripheral arteriosclerosis etc.), vascular hypertrophy, vascular hypertrophy or obliteration and organ damages after intervention (e.g., percutaneous transluminal coronary angioplasty, stenting, coronary angioscopy, intravascular ultrasound, dounce thrombolytic therapy etc.), vascular re-obliteration and restenosis after bypass, polycythemia, hypertension, organ damage and vascular hypertrophy after transplantation, rejection after transplantation, ocular diseases (e.g., glaucoma, ocular hypertension etc.), thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, obstructive thromboangiitis, ischemic cerebral circulatory disorder, Raynaud's disease, Berger disease etc.), metabolic and/or nutritional disorders (e.g., diabetes, impaired glucose tolerance, insulin resistance, hyperinsulinemia, diabetic nephropathy, diabetic retinopathy, diabetic neuropathy, obesity, hyperlipidemia, hypercholesterolemia, hyperuricacidemia, hyperkalemia, hypernatremia etc.), metabolic syndrome, nerve degeneration diseases (e.g., Alzheimer's disease, Parkinson's syndrome, amyotrophic lateral sclerosis, AIDS encephalopathy etc.), central nervous system disorders (e.g., cerebral hemorrhage, cerebral infarction, their sequela and complication, head injury, spinal injury, cerebral edema, sensory malfunction, sensory functional disorder, autonomic nervous system disorder, autonomic nervous system malfunction, multiple sclerosis etc.), dementia, migraine, defects of memory, disorder of consciousness, amnesia, anxiety symptom, catatonic symptom, discomfort mental state, sleep disorder, agrypnia, sychopathies (e.g., depression, epilepsy, alcoholism etc.), inflammatory diseases (e.g., arthritis such as rheumatoid arthritis, osteoarthritis, rheumatoid myelitis, periostitis etc.; inflammation after operation and injury; remission of swelling; pharyngitis; cystitis; pneumonia; atopic dermatitis; inflammatory intestinal diseases such as Crohn's disease, ulcerative colitis etc.; meningitis; inflammatory ocular disease; inflammatory pulmonary disease such as pneumonia, pulmonary silicosis, pulmonary sarcoidosis, pulmonary tuberculosis etc.), allergic diseases (e.g., allergic rhinitis, conjunctivitis, gastrointestinal allergy, pollinosis, anaphylaxis etc.), chronic obstructive pulmonary disease, interstitial pneumonia, pneumocytis carinni pneumonia, collagen diseases (e.g., systemic lupus erythematodes, scleroderma, polyarteritis etc.), hepatic diseases (e.g., hepatitis including chronic hepatitis, hepatic cirrhosis etc.), portal hypertension, digestive system disorders (e.g., gastritis, gastric ulcer, gastric cancer, gastric disorder after operation, dyspepsia, esophageal ulcer, pancreatitis, colon polyp, cholelithiasis, hemorrhoidal disease, varices ruptures of esophagus and stomach etc.), blood and/or myelopoietic diseases (e.g., erythrocytosis, vascular purpura, autoimmune hemolytic anemia, disseminated intravascular coagulation syndrome, multiple myelopathy etc.), bone diseases (e.g., fracture, refracture, osteoporosis, osteomalacia, bone Paget's disease, sclerosing myelitis, rheumatoid arthritis, osteoarthritis of the knee and joint tissue dysfunction and the like caused by diseases similar to these etc.), solid tumor, tumors (e.g., malignant melanoma, malignant lymphoma, cancer of digestive organs (e.g., stomach, intestine etc.) etc.), cancer and cachexia following cancer, metastasis cancer, endocrinopathy (e.g., Addison's disease, Cushing's syndrome, pheochromocytoma, primary aldosteronism etc.), Creutzfeldt-Jakob disease, urinary organ and/or male genital diseases (e.g., cystitis, prostatic hypertrophy, prostatic cancer, sex infectious disease etc.), female disorders (e.g., climacteric disorder, gestosis, endometriosis, hysteromyoma, ovarian disease, breast disease, sex infectious disease etc.), disease relating to environment and occupational factors (e.g., radiation hazard, hazard by ultraviolet, infrared or laser beam, altitude sickness etc.), respiratory diseases (e.g., cold syndrome, pneumonia, asthma, pulmonary hypertension, pulmonary thrombosis and pulmonary embolism etc.), infectious diseases (e.g., viral infectious diseases with cytomegalovirus, influenza virus, herpes virus etc., rickettsiosis, bacterial infectious disease etc.), toxemias (e.g., sepsis, septic shock, endotoxin shock, Gram-negative sepsis, toxic shock syndrome etc.), otorhinolaryngological diseases (e.g., Meniere's syndrome, tinnitus, dysgeusia, vertigo, disequilibrium, dysphagia etc.), skin-diseases (e.g., keloid, hemangioma, psoriasis etc.), intradialytic hypotension, myasthenia gravis, systemic diseases such as chronic fatigue syndrome and the like can be mentioned.

The compound of the present invention can be used in combination with an existing hypertension therapeutic drug such as an ACE inhibitor (captopril, enalapril maleate, alacepril, delapril hydrochloride, imidapril hydrochloride, quinapril hydrochloride, cilazapril, temocapril hydrochloride, trandolapril, benazepril hydrochloride, perindopril, lisinopril, etc.), ARB (losartan potassium, candesartan cilexetil, valsartan, TAK-536, TAK-491, irbesartan, telmisartan, eprosartan, olmesartan medoxomil, etc.), an aldosterone receptor antagonist (spironolactone, eplerenone, etc.), a Ca-ion channel inhibitor (verapamil hydrochloride, diltiazem hydrochloride, nifedipine, amlodipine hydrochloride, azelnidipine, aranidipine, efonidipine hydrochloride, cilnidipine, nicardipine hydrochloride, nisoldipine, nitrendipine, nilvadipine, barnidipine hydrochloride, felodipine, benidipine hydrochloride, manidipine hydrochloride, etc.), diuretic (trichlormethiazide, hydrochlorothiazide, benzylhydrochlorothiazide, indapamide, tripamide, meticrane, mefruside, furosemide, triamterene, chlorthalidon etc.), a β-blocker (propranolol hydrochloride, atenolol, metoprolol tartrate, bisoprolol fumarate, etc.), an α,β-blocker (carvedilol, etc.), or the like.

Moreover, the compound of the present invention can be also used in combination with an antithrombotic drug such as heparin sodium, heparin calcium, warfarin calcium (Warfarin), a blood coagulation factor Xa inhibitor, and a drug having a function of balance correction in the coagulation-fibrinolysis system, an oral thrombin inhibitor, a thrombolytic drug (tPA, urokinase, etc.), an antiplatelet drug [aspirin, sulfinpyrazone (Anturane), dipyridamol (Persantine), ticlopidine hydrochloride (Panaldine), clopidogrel, cilostazol (Pletal), GPIIb/IIIa antagonist (ReoPro, etc.)], or the like. Also, the compound can be used in combination with a lipid lowering drug or a cholesterol lowering drug. Examples thereof include a squalene synthase inhibitor (lapaquistat acetate etc.), fibrate (clofibrate, benzafibrate, gemfibrozil, etc.), nicotinic acid, its derivatives and analogs (acipimox, probucol, etc.), a bile acid binding resin (cholestyramine, colestipol, etc.), an omega-3 polyunsaturated fatty acid (EPA (eicosapentaenoic acid), DHA (docosahexaenoic acid), or a mixture thereof etc.), a compound inhibiting cholesterol absorption (sitosterol, neomycin, etc.), and a squalene epoxidase inhibitor (NB-598 and its analogs, etc.). Furthermore, other possible combination components are an oxidosqualene-lanosterol cyclase, for example, a decalin derivative, an azadecalin derivative, an indane derivative and the like. Combination with a HMG-CoA reductase (3-hydroxy-3-methylglutaryl coenzyme A reductase) inhibitor (atorvastatin calcium hydrate, pravastatin sodium, simvastatin, itavastatin, lovastatin, fluvastatin, etc.) is also possible.

The compound of the present invention can also be used in combination with a therapeutic drug for diabetes or a therapeutic drug for diabetic complications. For example, the compound of the present invention can be used in combination with an insulin preparation, an insulin sensitivity improving drug [pioglitazone hydrochloride, rosiglitazone, etc.], an α-glucosidase inhibitor [voglibose, acarbose, miglitol, emiglitate etc.], biguanide [phenformin, metformin, buformine etc.], insulin secretagogue [tolbutamide, glibenclamide, gliclazide, nateglinide, mitiglinide, glimepiride etc.], a dipeptidylpeptidase IV is inhibitor [Alogliptin benzoate, Vidagliptin (LAF237), P32/98, Saxagliptin (BMS-477118) etc.], Kinedak, Penfill, Humulin, Euglucon, Glimicron, Daonil, Novolin, Monotard, Glucobay, Dimelin, Rastinon, Bacilcon; Deamelin S, Iszilin family, or the like.

In addition to that, the compound can be also used together with other pharmaceutical components, including a bone disease medicine, a myocardial protective drug, a coronary artery disease medicine, a chronic cardiac failure medicine, a hypothyroidism medicine, a nephrotic syndrome medicine, a chronic renal failure medicine, a gynecological disease medicine or an infection medicine, or the like.

The administration mode may be exemplified by (1) administration of a single preparation obtained by simultaneously formulating the compound of the present invention and the combination drug, (2) simultaneous administration through the same administration route of two preparations obtained by separately formulating the compound of the present invention and the combination drug, (3) administration with a time interval through the same administration route of two preparations obtained by separately formulating the compound of the present invention and the combination drug, (4) simultaneous administration through different administration routes of two preparations obtained by separately formulating the compound of the present invention and the combination drug, (5) administration with a time interval through different administration routes of two preparations obtained by separately formulating the compound of the present invention and the combination drug (for example, administration in order of the compound of the present invention and then the combination drug, or administration in the reverse order), or the like. The amount of the combination drug to be administered can be appropriately selected with reference to the clinically used dosage. The mixing ratio of the compound of the present invention and the combination drug can be appropriately selected in accordance with the subject of administration, administration route, disease to be treated, symptoms, combination, and the like.

The compound of the present invention can be also used in combination with, for example, gene therapy involving VEGF, TNFα or the like, or therapeutic methods involving various antibody medicines or the like.

The compound of the present invention can be safely administered individually, or according to ordinary methods (for example, methods described in the Japanese Pharmacopeia, etc.), as pharmaceutical compositions mixed with pharmaceutically acceptable carriers, for example, a tablet (including a sugar-coated tablet and a film-coated tablet), a film, a powder, a granule, a capsule, a liquid, an emulsion, a suspension, an injectable preparation, a suppository, a sustained release preparation, a patch and the like, either orally or parenterally (e.g., topical, rectal, intravenous administration, etc.).

The dosage form of the aforementioned pharmaceutical preparation may be exemplified by oral preparations such as a tablet (including a sublingual tablet and a buccal disintegration tablet), a film (including a buccal disintegration film), a capsule (including a soft capsule and a microcapsule), a granule, a powder, a troche, a syrup, an emulsion, a suspension and the like; and parenteral preparations such as an injectable preparation (e.g., a subcutaneous injectable preparation, an intravenous injectable preparation, intramuscular injectable preparation, intraperitoneal injectable preparation, a drip infusion), external preparation (e.g., a percutaneous preparation, an ointment), a suppository (e.g., a rectal suppository, a vaginal suppository), a pellet, a transnasal preparation, a transpulmonary preparation (inhalant), an eye drop and the like.

These preparations may be controlled release preparations such as a rapid release preparation, a sustained release preparation or the like (e.g., a sustained release microcapsule).

The content of the compound of the present invention in the pharmaceutical composition is about 0.01 to 100% by weight of the entire composition.

The amount of administration of the compound of the present invention may vary depending on the subject of administration, administration route, subject disease or the like; however, in the case of administering orally to an adult as a hypertension medicine, the amount of administration is about 0.0005 to 2 mg/kg of body weight, preferably about 0.001 to 1 mg/kg of body weight, and more preferably about 0.001 to 0.5 mg/kg of body weight, in terms of compound (I), the active ingredient, possibly once to several times a day.

The aforementioned pharmaceutically acceptable carrier may be exemplified by various organic or inorganic carrier materials that are conventionally used as preparation materials, for example, excipient, gliding agent, binding agent and disintegrant for solid preparations; or solvent, solution aid, suspending agent, isotonic agent, buffering agent, soothing agent and the like for liquid preparations. Further, if necessary, additives such as preservative, antioxidant, colorant, sweetening agent, adsorbing agent, wetting agent and the like can be also used.

Examples of the excipient include lactose, white sugar, D-mannitol, starch, corn starch, crystalline cellulose, light silicic anhydride and the like.

Examples of the gliding agent include magnesium stearate, calcium stearate, talc, colloidal silica and the like.

Examples of the binding agent include crystalline cellulose, white sugar, D-mannitol, dextrin, hydroxypropylcellulose, hydroxypropylmethylcellulose, polyvinylpyrrolidone, starch, sucrose, gelatin, methylcellulose, carboxymethylcellulose sodium and the like.

Examples of the disintegrant include starch, carboxymethylcellulose, carboxymethylcellulose calcium, carboxymethylstarch sodium, L-hydroxypropylcellulose and the like.

Examples of the solvent include water for injection, alcohol, propylene glycol, Macrogol, sesame oil, corn oil, olive oil and the like.

Examples of the dissolution aid include polyethylene glycol, propylene glycol, D-mannitol, benzyl benzoate, ethanol, trisaminomethane, cholesterol, triethanolamine, sodium carbonate, sodium citrate and the like.

Examples of the suspending agent include surfactants such as stearyl triethanolamine, sodium lauryl sulfate, laurylaminopropionic acid, lecithin, benzalkonium chloride, benzetonium chloride, glycerin monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; and the like.

Examples of the isotonic agent include glucose, D-sorbitol, sodium chloride, glycerin, D-mannitol and the like.

Examples of the buffering agent, include buffer solutions of phosphates, acetates, carbonates, citrates and the like.

Examples of the soothing agent include benzyl alcohol and the like.

Examples of the preservative include parahydroxybenzoic acid esters, chlorobutanol, benzyl alcohol, phenethyl alcohol, dehydroacetic acid, sorbic acid and the like.

Examples of the antioxidant include sulfites, ascorbic acid, α-tocopherol and the like.

Examples of the colorant include water-soluble Food coal tar dyes (e.g., Food dyes such as Food Red No. 2 and No. 3, Food Yellow No. 4 and No. 5, Food Blue No. 1 and No. 2, and the like), water-insoluble lake dyes (e.g., aluminum salts of the aforementioned water-soluble Food coal tar dyes), natural dyes (e.g., β-carotene, chlorophyll, red iron oxide) and the like.

Examples of the sweetening agent include saccharin sodium, dipotassium glycyrrhizinate, aspartame, stevia and the like.

### EXAMPLE

The present invention is explained in detail in the following by referring to Reference Examples, Examples, Preparation Examples and Experimental Examples, which are not to be construed as limitative. Of the synthesis starting materials used in Reference Examples and Examples, synthetic methods of known compounds are omitted.

“Room temperature” in the following Reference Examples and Examples represents a temperature of about 10° C. to about 35° C., and “%” represents weight % unless otherwise stated. Provided that, yield represents mol/mol %.
¹H-NMR spectra were measured with a Varian GEMINI 200 (200 MHz) spectrometer, a MERCURY 300 (300 MHz) spectrometer or a BRUKER ADVANCE 300 spectrometer (300 MHz) using tetramethylsilane as an internal standard. All of the 8 values are represented in ppm.
LC/MS spectra were measured under the following conditions. Equipment: Agilent 1100 HPLC (Gilson 215 autosampler)/Waters ZQ, or Waters 2795/ZQ
Column:CapcellPak C18UG120, manufactured by Shiseido Co., Ltd. (S-3 μm, 1.5×35 mm)
Solvent: Solution A (0.05% trifluoroacetic acid-containing water), Solution B (0.04% trifluoroacetic acid-containing water)
Gradient cycle: 0.00 min (A/B=90/10), 2.00 min (A/B=5/95), 2.75 min (A/B=5/95), 2.76 min (A/B=90/10), 3.45 min (A/B=90/10)
Flow rate: 0.5 ml/min
Detection: UV (220 nm)
Mass spectrum: electrospray ionization (ESI)

Reverse-phase HPLC analysis was carried out on an YMC CombiPrep Pro C18 (50×20 mm, S-5 μm) Column or YMC Hydrosphere C18 (30×75 mm) Column using a Gilson UniPoint system, and eluted with 0.1% trifluoroacetic acid-containing acetonitrile/water (5:95 to 100:0 or 2:98 to 100:0).

Other symbols used in the present text indicate the following meanings.
s: singlet, d: doublet, t: triplet, q: quartet, dd: double doublet, dt: double triplet, td: triple doublet, dq: double quartet, ddd: double double doublet, m: multiplet, br: broad.
Me: methyl, Et: ethyl, nPr: n-propyl, iPr: isopropyl, nBu: n-butyl, iBu: isobutyl, tBu: tert-butyl, Ph: phenyl, Ac: acetyl, Boc: tert-butoxycarbonyl, Cbz: benzyloxycarbonyl.
DEAD: diethyl azodicarboxylate, DMA: N,N-dimethylacetamide, DME: 1,2-dimethoxyethane, DMF: N,N-dimethylformamide, DMSO: dimethyl sulfoxide, THF: tetrahydrofuran.
BINAP: 2,2′-bis(diphenylphosphino)-1,1′-binaphthyl, CDI: N,N′-carbonyldiimidazole, DBU: 1,8-diazabicyclo[5.4.0]-7-undecene, DCC: dicyclohexylcarbodiimide, DMAP: 4-(dimethylamino)pyridine, dppf: 1,1′-bis(diphenylphosphino)ferrocene, DSC: N,N′-disuccinimidyl carbonate, HOBt: 1-hydroxybenzotriazole, NBS: N-bromosuccinimide, Pd2(dba)₃: tris(dibenzylideneacetone)dipalladium(0), TBAF: tetra-n-butylammonium fluoride, TFA: trifluoroacetic acid, TMEDA: N,N,N′,N′-tetramethylethylenediamine, WSC.HCl: 1-ethyl-3-[3-(dimethylamino)propyl]carbodiimide hydrochloride.

### REFERENCE EXAMPLE

### Reference Example 1

### Ethyl1-(2-methoxybenzyl)-2-phenyl-1H-pyrrole-3-carboxylate

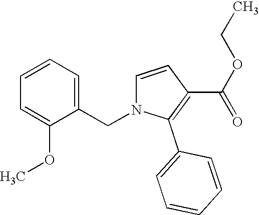

To a solution of ethyl2-phenyl-1H-pyrrole-3-carboxylate (330 mg), 2-methoxybenzyl chloride (288 mg) and DMF (3 ml), was added sodium hydride (60% in oil) (74 mg) with ice cooling. After stirring at 0° C. for 30 min and at room temperature for 2 hr, the reaction mixture was poured into a saturated aqueous sodium bicarbonate solution and extracted with ethyl acetate. The extract was washed with brine and dried over anhydrous sodium sulfate, and then the solvent was evaporated in vacuo. The residue was subjected to silica gel column chromatography, and the fraction eluted with ethyl acetate-hexane (1:4) was concentrated in vacuo to give the desired product (320 mg) as an amorphous solid.

¹H-NMR (CDCl₃) δ 1.11 (3H, t), 3.73 (3H, s), 4.10 (2H, q), 4.88 (2H, s), 6.44-6.56 (2H, m), 6.65-6.79 (3H, m), 7.15-7.61 (6H, m)

In the same manner as in Reference Example 1, the following compounds (Reference Examples 2 to 3) were obtained.

### Reference Example 2

### Ethyl1-benzyl-2-phenyl-1H-pyrrole-3-carboxylate

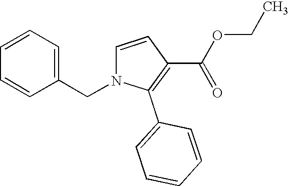

¹H-NMR (CDCl₃) δ 1.11 (3H, t), 4.10 (2H, q), 4.91 (2H, s), 6.70 (2H, dd), 6.88-7.01 (2H, m), 7.17-7.42 (8H, m)

### Reference Example 3

### Ethyl1-(4-methoxybenzyl)-2-phenyl-1H-pyrrole-3-carboxylate

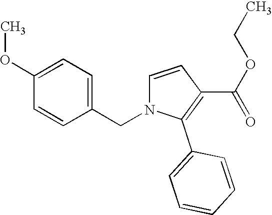

¹H-NMR (CDCl₃) δ 1.10 (3H, t), 3.77 (3H, s), 4.09 (2H, q), 4.84 (2H, s), 6.52-6.99 (6H, m), 7.23-7.41 (5H, m)

### Reference Example 4

### Ethyl1-(3-methoxybenzyl)-2-phenyl-1H-pyrrole-3-carboxylate

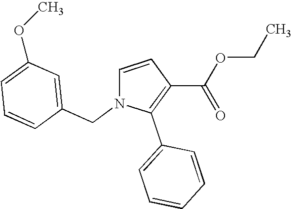

To a solution of ethyl2-phenyl-1H-pyrrole-3-carboxylate (215 mg), 3-methoxybenzyl bromide (188 mg) and DMF (5 ml), was added potassium carbonate (415 mg). After stirring at 80° C. for 12 hr, the reaction mixture was poured into water and extracted with ethyl acetate. The extract was washed with brine and dried over anhydrous sodium sulfate, and then the solvent was evaporated in vacuo. The residue was subjected to silica gel column chromatography, and the fraction eluted with ethyl acetate-hexane (1:9) was concentrated in vacuo to give the desired product (340 mg) as an amorphous solid.

¹H-NMR (CDCl₃) δ 1.11 (3H, t), 3.75 (3H, s), 4.10 (2H, q), 4.91 (2H, s), 6.62-6.90 (5H, m), 7.18-7.40 (6H, m)

### Reference Example 5

### Ethyl2-methyl-1-(4-nitrophenyl)-1H-pyrrole-3-carboxylate

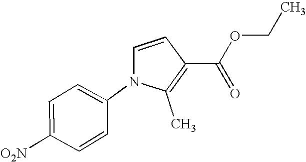

To a solution of ethyl2-methyl-1H-pyrrole-3-carboxylate (280 mg), 4-fluoronitrobenzene (366 mg) and DMF (5 ml), was added potassium carbonate (415 mg). After stirring at 80° C. for 12 hr, the reaction mixture was poured into water and extracted with ethyl acetate. The extract was washed with brine and dried over anhydrous sodium sulfate, and then the solvent was evaporated in vacuo. The residue was subjected to silica gel column chromatography, and the fraction eluted with ethyl acetate-hexane (1:9) was concentrated in vacuo to give the desired product (340 mg) as an amorphous solid.

¹H-NMR (CDCl₃) δ 1.37 (3H, t), 2.52 (3H, s), 4.31 (2H, q), 6.70-6.75 (2H, m), 7.42-7.52 (2H, m), 8.32-8.42 (2H, m)

In the same manner as in Reference Example 5, the following compounds (Reference Examples 6 to 8) were obtained.

### Reference Example 6

### Ethyl1-(2-nitrophenyl)-2-phenyl-1H-pyrrole-3-carboxylate

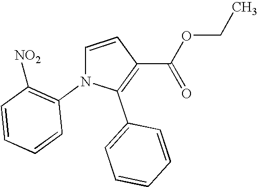

¹H-NMR (CDCl₃) δ 1.18 (3H, t), 4.17 (2H, q), 6.77 (1H, d), 6.89 (1H, d), 7.16-7.32 (6H, m), 7.40-7.48 (1H, m), 7.51-7.60 (1H, m), 7.82 (1H, dd)

### Reference Example 7

### Ethyl1-(4-nitrophenyl)-2-phenyl-1H-pyrrole-3-carboxylate

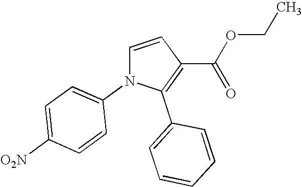

¹H-NMR (CDCl₃) δ 1.19 (3H, t), 4.18 (2H, q), 6.94 (1H, d), 6.88-7.02 (1H, m), 7.17-7.33 (7H, m), 8.13 (2H, d)

### Reference Example 8

### Ethyl1-(5-methoxy-2-nitrophenyl)-2-phenyl-1H-pyrrole-3-carboxylate

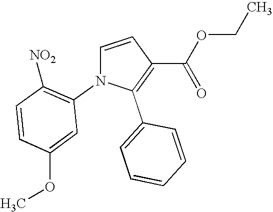

¹H-NMR (CDCl₃) δ 1.18 (3H, t), 3.80 (3H, s), 4.17 (2H, q), 6.74-6.78 (2H, m), 6.85-6.91 (2H, m), 7.15-7.28 (5H, m), 7.88 (1H, d)

### Reference Example 9

### Ethyl1-benzyl-3-phenyl-1H-pyrazole-4-carboxylate and ethyl1-benzyl-5-phenyl-1H-pyrazole-4-carboxylate

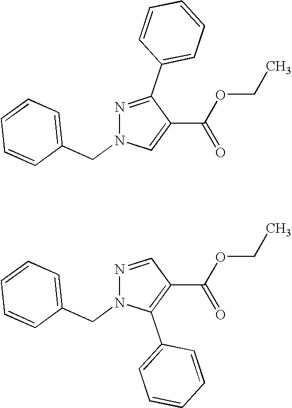

A solution of ethyl benzoylacetate (3.00 g) and N,N-dimethylacetamide dimethylacetal (2.49 ml) in toluene (50 ml) was heated under reflux for 15 hr. The reaction mixture was concentrated in vacuo, and the residue was dissolved in ethanol (50 ml). Benzylhydrazine hydrochloride (2.72 g) and triethylamine (2.39 ml) were added thereto, and the mixture was heated under reflux for 3 hr. The reaction mixture was poured into water and extracted with ethyl acetate. The extract was washed with brine and dried over anhydrous sodium sulfate, and then the solvent was evaporated in vacuo. The residue was subjected to silica gel column chromatography, and the fraction eluted with ethyl acetate-hexane (1:49 to 1:0) was concentrated in vacuo to give ethyl1-benzyl-3-phenyl-1H-pyrazole-4-carboxylate (0.815 g) as an oil and ethyl1-benzyl-5-phenyl-1H-pyrazole-4-carboxylate as crystals. The resulting crystals were recrystallized from ethyl acetate-hexane and purified (2.25 g).

### Ethyl1-benzyl-3-phenyl-1H-pyrazole-4-carboxylate

¹H-NMR (CDCl₃) δ 1.25 (3H, t), 4.21 (2H, q), 5.34 (2H, s), 7.29-7.44 (8H, m), 7.78 (2H, dd), 7.91 (1H, s)

### Ethyl1-benzyl-5-phenyl-1H-pyrazole-4-carboxylate

¹H-NMR (CDCl₃) δ 1.15 (3H, t), 4.14 (2H, q), 5.18 (2H, s), 7.00 (2H, dd), 7.25 (1H, s), 7.25-7.29 (5H, m), 7.38-7.49 (3H, m), 8.06 (1H, s)

### Reference Example 10

### Ethyl2-(3-(benzyloxy)benzoyl)-4-oxopentanoate

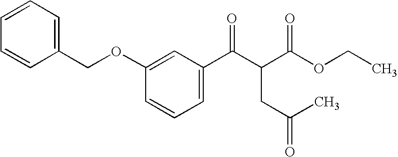

Chloroacetone (10.14 g) was added to a suspension of ethyl3-[3-(benzyloxy)phenyl)-3-oxopropanoate (29.72 g), potassium carbonate (27.54 g), potassium iodide (3.31 g) and acetone (120 ml) at room temperature. The reaction mixture was heated under reflux for 2 hr, and then the solution was concentrated in vacuo, diluted with water and extracted with ethyl acetate. The extract was washed successively with water and brine and dried over anhydrous magnesium sulfate, and then the solvent was evaporated in vacuo. The residue was subjected to silica gel column chromatography, and the fraction eluted with ethyl acetate-hexane (1:3) was concentrated in vacuo to give the desired product (31.41 g) as an oil.

¹H-NMR (CDCl₃) δ 1.16 (3H, t), 2.23 (3H, s), 3.16 (2H, ddd), 4.13 (2H, q), 4.86 (1H, t), 5.11 (2H, s), 7.18-7.22 (1H, m), 7.31-7.46 (6H, m), 7.61-7.64 (2H, m)

### Reference Example 11

### Ethyl2-(3-(benzyloxy)phenyl)-5-methyl-1-phenyl-1H-pyrrole-3-carboxylate

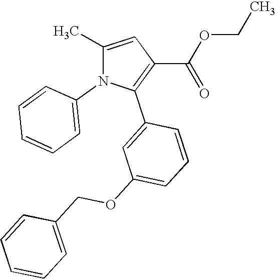

A solution of ethyl2-[3-(benzyloxy)benzoyl]-4-oxopentanoate (12.00 g), aniline (4.10 g), p-toluenesulfonic acid hydrate (515 mg) and ethanol (170 ml) was heated under reflux for 15 hr, and then the mixture was poured into water and extracted with ethyl acetate. The extract was washed successively with 0.1 N hydrochloric acid, a saturated aqueous sodium bicarbonate solution, water and brine and dried over anhydrous magnesium sulfate, and then the solvent was evaporated in vacuo. The residue was subjected to silica gel column chromatography, and the fraction eluted with ethyl acetate-hexane (1:6) was concentrated in vacuo to give the desired product (13.02 g) as an oil.

¹H-NMR (CDCl₃) δ 1.17 (3H, t), 2.08 (3H, s), 4.15 (2H, q), 4.86 (2H, s), 6.54 (1H, s), 6.76-6.81 (3H, m), 6.99-7.11 (3H, m), 7.24-7.36 (8H, m)

In the same manner as in Reference Example 11, the following compounds (Reference Examples 12 to 18) were obtained by reacting ethyl2-benzoyl-4-oxopentanoate with various aniline derivatives.

### Reference Example 12

### Ethyl1-(3-bromophenyl)-5-methyl-2-phenyl-1H-pyrrole-3-carboxylate

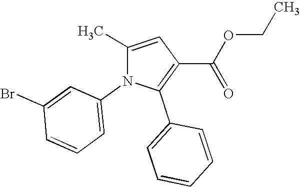

¹H-NMR (CDCl₃) δ 1.15 (3H, t), 2.09 (3H, d), 4.14 (2H, q), 6.54 (1H, s), 6.83-7.02 (1H, m), 7.09-7.26 (7H, m), 7.39 (1H, dd)

### Reference Example 13

### Ethyl1-(3-methoxyphenyl)-5-methyl-2-phenyl-1H-pyrrole-3-carboxylate

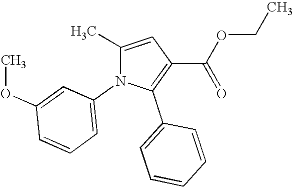

¹H-NMR (CDCl₃) δ 1.16 (3H, t), 2.11 (3H, s), 3.66 (3H, s), 4.14 (2H, q), 6.46-6.59 (2H, m), 6.65 (1H, d), 6.80 (1H, s), 7.13-7.28 (6H, m)

### Reference Example 14

### Ethyl1-(3,4-dimethoxyphenyl)-5-methyl-2-phenyl-1H-pyrrole-3-carboxylate

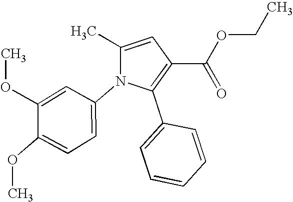

¹H-NMR (CDCl₃) δ 1.16 (3H, t), 2.11 (3H, d), 3.66 (3H, s), 3.85 (3H, s), 4.14 (2H, q), 6.46 (1H, d), 6.53 (1H, d), 6.64-6.78 (2H, m), 7.17 (5H, s)

### Reference Example 15

### Ethyl1-[2-(benzyloxy)phenyl]-5-methyl-2-phenyl-1H-pyrrole-3-carboxylate

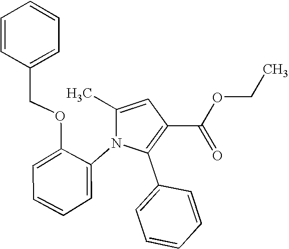

¹H-NMR (CDCl₃) δ 1.16 (3H, t), 2.02 (3H, s), 4.14 (2H, q), 4.93-5.06 (2H, m), 6.56 (1H, d), 6.82-6.90 (2H, m), 7.02 (1H, dd), 7.11-7.23 (7H, m), 7.25-7.35 (4H, m)

### Reference Example 16

### Ethyl1-(2,2-difluoro-1,3-benzodioxol-4-yl)-5-methyl-2-phenyl-1H-pyrrole-3-carboxylate

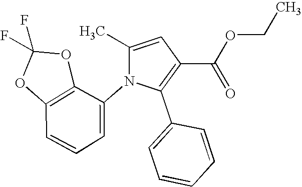

¹H-NMR (CDCl₃) δ 1.15 (3H, t), 2.11 (3H, s), 4.14 (2H, q), 6.58 (1H, s), 6.75 (1H, dd), 6.88-7.06 (1H, m), 7.18 (5H, s), 7.25 (1H, s)

### Reference Example 17

### Ethyl1-(2-{[(tert-butoxycarbonyl)amino]methyl}phenyl)-5-methyl-2-phenyl-1H-pyrrole-3-carboxylate

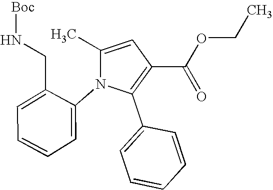

¹H-NMR (CDCl₃) δ 1.12-1.22 (3H, m), 1.34-1.46 (9H, m), 1.98 (3H, s), 3.59-3.74 (1H, m) 3.89 (1H, dd), 4.05-4.37 (3H, m), 6.59 (1H, s), 7.15-7.21 (5H, m), 7.23-7.33 (4H, m)

### Reference Example 18

### Ethyl1-(2,3-dimethoxyphenyl)-5-methyl-2-phenyl-1H-pyrrole-3-carboxylate

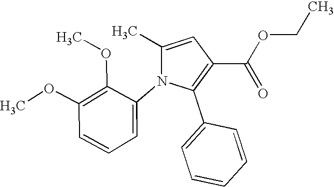

¹H-NMR (CDCl₃) δ 1.16 (3H, t), 2.04 (3H, s), 3.57 (3H, s), 3.81 (3H, s), 4.14 (2H, q), 6.55 (1H, s), 6.67 (1H, dd), 6.82-6.87 (1H, m), 6.90-6.97 (1H, m), 7.14-7.27 (5H, m)

### Reference Example 19

### Methyl5-acetyl-1,2-diphenyl-1H-pyrrole-3-carboxylate and methyl4-acetyl-1,2-diphenyl-1H-pyrrole-3-carboxylate

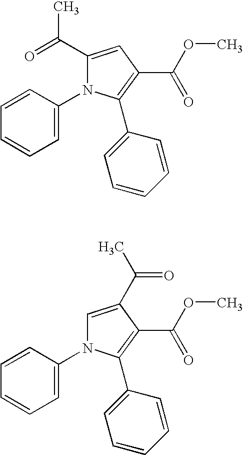

A solution of methyl1,2-diphenyl-1H-pyrrole-3-carboxylate (1.6 g), isopropenyl acetate (3.5 g), methanesulfonic acid (0.4 ml) and 1,2-dichloroethane (20 ml) was heated at 80° C. and stirred for 3 days. The reaction mixture was poured into water and the mixture was extracted with chloroform. The extract was washed with a 10% aqueous sodium bicarbonate solution and dried over anhydrous sodium sulfate, and the solvent was then evaporated in vacuo. The residue was subjected to silica gel column chromatography, and the fraction eluted with ethyl acetate-hexane (1:4) was m concentrated in vacuo to give both of methyl5-acetyl-1,2-diphenyl-1H-pyrrole-3-carboxylate (290 mg) and methyl4-acetyl-1,2-diphenyl-1H-pyrrole-3-carboxylate (350 mg) as an amorphous solid.

### Methyl5-acetyl-1,2-diphenyl-1H-pyrrole-3-carboxylate

¹H-NMR (CDCl₃) δ 2.45 (3H, s), 3.71 (3H, s), 6.95-7.29 (10H, m), 7.58 (1H, s)

### Methyl4-acetyl-1,2-diphenyl-1H-pyrrole-3-carboxylate

¹H-NMR (CDCl₃) δ 2.48 (3H, s), 3.76 (3H, s), 7.00-7.13 (2H, m), 7.15-7.26 (6H, m), 7.28-7.31 (2H, m), 7.41 (1H, s)

### Reference Example 20

### Methyl1-(3-methoxypropyl)-4,5-diphenyl-1H-pyrrole-3-carboxylate

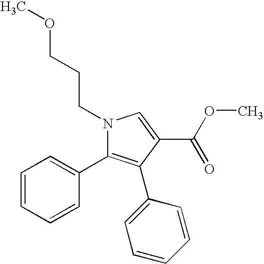

A solution of methyl4,5-diphenyl-1H-pyrrole-3-carboxylate (200 mg), 1-bromo-3-methoxypropane (132 mg) and DMF (2 ml) was ice-cooled, and sodium hydride (60% in oil) (40 mg) was added thereto. After stirring at room temperature for 1 hr, the mixture was poured into an ice-cooled saturated aqueous sodium bicarbonate solution and extracted with ethyl acetate. The extract was dried over anhydrous sodium sulfate and the solvent was evaporated in vacuo. The residue was subjected to basic silica gel column chromatography, and the fraction eluted with ethyl acetate-hexane (1:4) was concentrated in vacuo to give the desired product (140 mg) as an amorphous solid.

¹H-NMR (CDCl₃) δ 1.74-1.89 (2H, m), 3.18-3.30 (5H, m), 3.69 (3H, s), 3.96 (2H, t), 7.05-7.52 (11H, m)

### Reference Example 21

### Methyl5-cyclohexyl-1-phenyl-1H-pyrazole-4-carboxylate

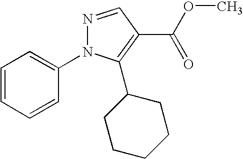

A solution of methyl3-cyclohexyl-3-oxopropionate (5.50 g), N,N-dimethylformamide dimethylacetal (5.30 g) and toluene (50 ml) was heated under reflux for 4 hr, and the reaction mixture was concentrated in vacuo. To the residue were added phenylhydrazine (2.95 g) and ethanol (50 ml), and the mixture was heated under reflux overnight. The reaction mixture was concentrated in vacuo, and the crystals were collected by filtration to give the desired product (4.90 g).

¹H-NMR (CDCl₃) δ 1.18 (2H, t), 1.29 (1H, s), 1.58 (3H, s), 1.78 (2H, s), 2.14 (2H, d), 2.83-2.96 (1H, m), 3.86 (3H, s), 7.31-7.39 (2H, m), 7.45-7.55 (3H, m), 8.02 (1H, s)

### Reference Example 22

### Methyl1-(3-morpholinophenyl)-5-phenyl-1H-imidazole-4-carboxylate

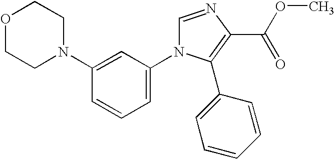

A solution of methyl3-bromo-2-isocyano-3-phenylacrylate (1.80 g), 3-morpholinoaniline (1.45 g), triethylamine (1.37 g) and DMF (20 ml) was stirred for 2 days at room temperature under an argon atmosphere. Then, the mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to basic silica gel column chromatography, the fraction eluted with ethyl acetate-hexane (1:1 to 1:0) was concentrated in vacuo, and the crystals were collected by filtration to give the desired product (1.02 g). A portion thereof was recrystallized from ethyl acetate-hexane and taken as a sample for analysis.

¹H-NMR (CDCl₃) δ 2.94-2.97 (4H, m), 3.75-3.79 (4H, m), 3.84 (3H, s), 6.44 (1H, t), 6.59-6.62 (1H, m), 6.81-6.84 (1H, m), 7.21 (1H, t), 7.24-7.32 (5H, m), 7.74 (1H, s)

MS (ESI+, m/e) 364 (M+1)

### Reference Example 23

### Methyl1-(2,3-dimethoxyphenyl)-5-phenyl-1H-imidazole-4-carboxylate

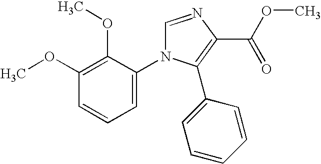

A solution of methyl3-bromo-2-isocyano-3-phenylacrylate (1.80 g), 2,3-dimethoxyaniline (1.24 g), triethylamine (1.37 g) and DMF (20 ml) was stirred at room temperature for 2 days and at 70° C. for 10 hr under an argon atmosphere. Then, the mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to silica gel column chromatography, the fraction eluted with ethyl acetate-hexane (1:1 to 1:0) was concentrated in vacuo, and the crystals were collected by filtration to give the desired product (160 mg).

¹H-NMR (CDCl₃) δ 3.60 (3H, s), 3.84 (3H, s), 3.85 (3H, s), 6.62 (1H, dd), 6.90 (1H, dd), 6.95 (1H, t), 7.25-7.27 (5H, m), 7.64 (1H, s)

### Reference Example 24

### Methyl5-phenyl-1-[(1S)-1-phenylethyl]-1H-imidazole-4-carboxylate

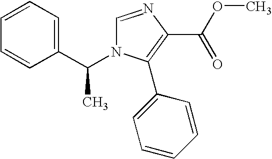

A solution of methyl3-bromo-2-isocyano-3-phenylacrylate (1.80 g), (1S)-1-phenylethylamine (984 mg), triethylamine (1.37 g) and DMF (20 ml) was stirred at room temperature for 3 days under an argon atmosphere, and then the mixture was poured into water. The reaction mixture was weakly acidified (pH 3) with 2 N hydrochloric acid and extracted with ethyl acetate. The extract was washed successively with a saturated aqueous sodium bicarbonate solution, water and brine and dried over anhydrous magnesium sulfate, and then the solvent was evaporated in vacuo. The residue was subjected to silica gel column chromatography, the fraction eluted with ethyl acetate-hexane-methanol (1:1:0 to 20:0:1) was concentrated in vacuo, and the crystals were collected by filtration to give the desired product (1.34 g). A portion thereof was recrystallized from ethyl acetate-hexane and taken as a sample for analysis.

¹H-NMR (CDCl₃) δ 1.81 (3H, d), 3.77 (3H, s), 5.16 (1H, q), 6.94-6.97 (2H, m), 7.18-7.20 (2H, m), 7.25-7.32 (3H, m), 7.35-7.43 (3H, m), 7.68 (1H, s)

MS (ESI+, m/e) 307 (M+1)

In the same manner as in Reference Example 24, the following compounds (Reference Examples 25 to 27) were obtained.

### Reference Example 25

### Methyl5-phenyl-1-[(1R)-1-phenylethyl]-1H-imidazole-4-carboxylate

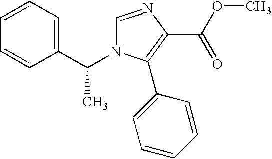

¹H-NMR (CDCl₃) δ 1.81 (3H, d), 3.77 (3H, s), 5.16 (1H, q), 6.94-6.97 (2H, m), 7.18-7.20 (2H, m), 7.25-7.32 (3H, m), 7.36-7.45 (3H, m), 7.68 (1H, s)

MS (ESI+, m/e) 307 (M+1)

### Reference Example 26

### Methyl1-[(1R)-2,3-dihydro-1H-inden-1-yl]-5-phenyl-1H-imidazole-4-carboxylate

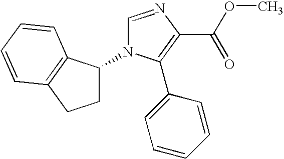

¹H-NMR (CDCl₃) δ 2.05-2.23’(1H, m), 2.52-2.63 (1H, m), 2.83-2.94 (1H, m), 3.05-3.14 (1H, m), 3.79 (3H, s), 5.45 (1H, t), 7.07 (1H, d), 7.21-7.32 (4H, m), 7.42-7.52 (5H, m)

MS (ESI+, m/e) 319 (M+1)

### Reference Example 27

### Methyl5-phenyl-1-[(1R)-1,2,3,4-tetrahydronaphthalen-1-yl]-1H-imidazole-4-carboxylate

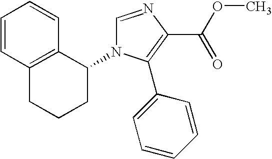

¹H-NMR (CDCl₃) δ 1.69-1.78 (1H, m), 1.88-2.02 (2H, m), 2.04-2.15 (1H, m), 2.77 (1H, dt), 2.92 (1H, ddd), 3.79 (3H, s), 5.16 (1H, t), 6.83 (1H, d), 7.10-7.25 (3H, m), 7.29 (1H, s), 7.41-7.51 (5H, m)

MS (ESI+, m/e) 333 (M+1)

### Reference Example 28

### Methyl1-(2,3-dihydro-1H-inden-2-yl)-5-phenyl-1H-imidazole-4-carboxylate

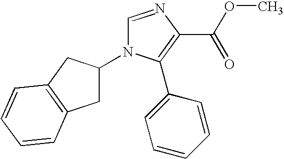

A solution of methyl3-bromo-2-isocyano-3-phenylacrylate (1.85 g), indan-2-amine (1.11 g), triethylamine (1.41 g) and DMF (20 ml) was stirred under argon atmosphere at room temperature for 3 days and poured into water. The mixture was weakly acidified (pH 3) with 2 N hydrochloric acid and extracted with ethyl acetate. The extract was washed successively with saturated aqueous sodium bicarbonate solution, water and brine, and dried over anhydrous magnesium sulfate, and the solvent was evaporated in vacuo. The residue was subjected to silica gel column chromatography, the fraction eluted with ethyl acetate-hexane (1:2 to 1:0) was concentrated in vacuo, and the crystals were collected by filtration to give the desired product (1.42 g). A part thereof was recrystallized from ethyl acetate-hexane to give a sample for analysis.

¹H-NMR (CDCl₃) δ 3.20 (2H, dd), 3.37 (2H, dd), 3.77 (3H, s), 4.72-4.81 (1H, m), 7.23 (4H, s), 7.37-7.40 (2H, m), 7.47-7.54 (4H, m)

MS (ESI+, m/e) 319 (M+1)

### Reference Example 29

### 2-[3-(Benzyloxy)phenyl]-5-methyl-1-phenyl-1H-pyrrole-3-carboxylic acid

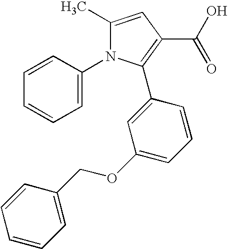

Ethyl2-[3-(benzyloxy)phenyl]-5-methyl-1-phenyl-1H-pyrrole-3-carboxylate (13.01 g) was dissolved in ethanol (90 ml), a 4 N aqueous sodium hydroxide solution (79 ml) was added thereto, and the mixture was heated under reflux for 5 hr. The reaction mixture was poured into water, and the mixture was weakly acidified (pH 3) with concentrated hydrochloric acid, and then extracted with ethyl acetate-THF (2:1). The extract was washed successively with water and brine and dried over anhydrous magnesium sulfate. Then, the solvent was evaporated in vacuo, and the crystals were collected by filtration to give the desired product (11.48 g). A portion thereof was recrystallized from THF-ethyl acetate and taken as a sample for analysis.

¹H-NMR (DMSO-d₆) δ 1.99 (3H, s), 4.90 (2H, s), 6.41 (1H, s), 6.69-6.80 (3H, m), 7.03-7.15 (3H, m), 7.29-7.40 (8H, m), 11.57 (1H, s)

### Reference Example 30

### 1,2-Diphenyl-1H-pyrrole-3-carboxylic acid

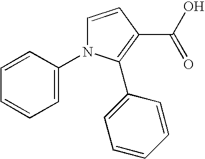

Methyl1,2-diphenyl-1H-pyrrole-3-carboxylate (3.7 g) was suspended in ethanol (100 ml) and THF (100 ml). A 1 N aqueous lithium hydroxide solution (13.3 ml) and a 1 N aqueous sodium hydroxide solution (40 ml) were added thereto, and the suspension was heated under reflux for 12 hr. Then, the reaction mixture was concentrated in vacuo and weakly acidified (pH 3) by adding 2 N hydrochloric acid to the remaining aqueous solution. The reaction mixture was extracted with ethyl acetate, and the extract was washed with brine and dried over anhydrous sodium sulfate. The solvent was then evaporated in vacuo, and the residue was recrystallized from chloroform-methanol-hexane (9:1:30) to give the desired product (3.3 g).

¹H-NMR (DMSO-d₆) δ 6.68 (1H, d), 6.99-7.46 (11H, m), 11.75 (1H, s)

### Reference Example 31

### 1-(2-Methoxybenzyl)-2-phenyl-1H-pyrrole-3-carboxylic acid

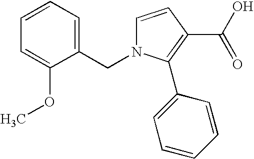

Ethyl1-(2-methoxybenzyl)-2-phenyl-1H-pyrrole-3-carboxylate (180 mg) was dissolved in ethanol (2 ml), and 15% lithium hydroxide (2 ml) was added thereto. After heating under reflux for 12 hr, the reaction mixture was concentrated in vacuo and weakly acidified (pH 3) by adding 2 N hydrochloric acid to the remaining aqueous solution. The reaction mixture was extracted with ethyl acetate, and the extract was washed with brine and dried over anhydrous sodium sulfate. Then, the solvent was evaporated in vacuo, and the residue was dried in vacuo to give the desired product (160 mg).

¹H-NMR (CDCl₃) δ 3.73 (3H, s), 4.86 (2H, s), 6.41-6.56 (2H, m), 6.60-6.82 (3H, m), 7.12-7.41 (6H, m), 11.51 (1H, br s)

In the same manner as in Reference Example 31, the following compounds (Reference Examples 32 to 38) were obtained.

### Reference Example 32

### 1-(3-Methoxybenzyl)-2-phenyl-1H-pyrrole-3-carboxylic acid

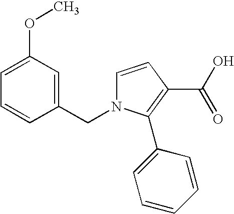

¹H-NMR (DMSO-d₆) δ 3.68 (3H, s), 4.89 (2H, s), 6.38-6.64 (2H, m), 6.77-6.95 (3H, m), 7.13-7.44 (6H, m), 11.52 (1H, s)

### Reference Example 33

### 1-(4-Methoxybenzyl)-2-phenyl-1H-pyrrole-3-carboxylic acid

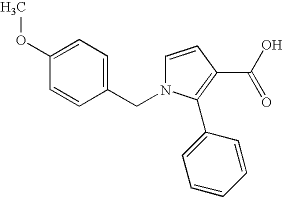

¹H-NMR (DMSO-d₆) δ 3.69 (3H, s), 4.87 (2H, s), 6.52 (1H, d), 6.67-7.01 (5H, m), 7.16-7.48 (5H, m), 11.50 (1H, br s)

### Reference Example 34

### 1-Benzyl-2-phenyl-1H-pyrrole-3-carboxylic acid

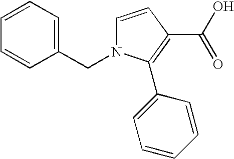

¹H-NMR (CDCl₃) δ 4.96 (2H, s), 6.56 (1H, d), 6.84 (2H, d), 6.95 (1H, d), 7.14-7.31 (5H, m), 7.31-7.39 (3H, m), 11.55 (1H, s)

### Reference Example 35

### 2-Methyl-1-(4-nitrophenyl)-1H-pyrrole-3-carboxylic acid

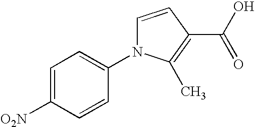

¹H-NMR (CDCl₃) δ 2.13 (3H, s), 6.13 (1H, br s), 6.73 (1H, d), 7.44 (2H, d), 8.35 (2H, d)

### Reference Example 36

### 1-(2-Nitrophenyl)-2-phenyl-1H-pyrrole-3-carboxylic acid

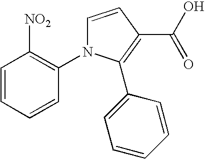

¹H-NMR (CDCl₃) δ 6.77 (1H, d), 6.91 (1H, d), 7.15-7.31 (6H, m), 7.41-7.59 (2H, m), 7.82 (1H, dd)

### Reference Example 37

### 1-(4-Nitrophenyl)-2-phenyl-1H-pyrrole-3-carboxylic acid

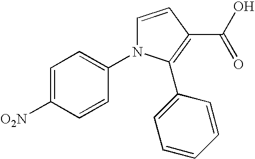

¹H-NMR (DMSO-d₆) δ 6.73 (1H, d), 7.14-7.48 (8H, m), 8.10-8.17 (2H, m), 11.90 (1H, br s)

### Reference Example 38

### 1-(5-Hydroxy-2-nitrophenyl)-2-phenyl-1H-pyrrole-3-carboxylic acid

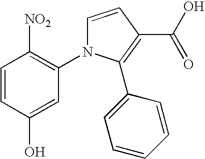

¹H-NMR (DMSO-d₆) δ 6.68 (1H, d), 6.82 (1H, d), 6.84-6.92 (1H, m), 7.02-7.05 (1H, m), 7.06-7.25 (6H, m), 11.25 (1H, s), 11.80 (1H, br s)

Note: During the hydrolysis of ethyl1-(5-methoxy-2-nitrophenyl)-2-phenyl-1H-pyrrole-3-carboxylate, the methoxy group was also removed, thus leaving the hydroxy group.

### Reference Example 39

### 1-(3-Bromophenyl)-5-methyl-2-phenyl-1H-pyrrole-3-carboxylic acid

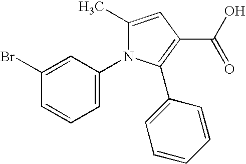

Ethyl1-(3-bromophenyl)-5-methyl-2-phenyl-1H-pyrrole-3-carboxylate (7.9 g) was suspended in ethanol (20 ml), a 15% aqueous lithium hydroxide solution (20 ml) was added thereto, and the suspension was heated under reflux for 24 hr. The reaction mixture was concentrated in vacuo, weakly acidified (pH 3) by adding 2 N hydrochloric acid to the remaining aqueous solution, and then extracted with ethyl acetate. The extract was washed with brine and dried over anhydrous sodium sulfate. Then, the solvent was evaporated in vacuo, and the crystals were collected by filtration to give the desired product (7.3 g).

¹H-NMR (DMSO-d₆) δ 2.02 (3H, s), 6.42 (1H, s), 7.11-7.21 (5H, m), 7.25-7.31 (1H, m), 7.40-7.43 (1H, m), 7.48-7.53 (1H, m), 11.62 (1H, br s)

In the same manner as in Reference Example 39, the following compounds (Reference Examples 40 to 45) were obtained.

### Reference Example 40

### 1-(3-Methoxyphenyl)-5-methyl-2-phenyl-1H-pyrrole-3-carboxylic acid

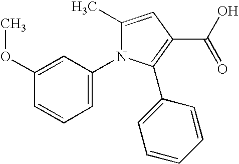

¹H-NMR (DMSO-d₆) δ 2.03 (3H, s), 3.64 (3H, s), 6.41 (1H, s), 6.59-6.78 (2H, m), 6.83-6.89 (1H, m), 7.04-7.27 (6H, m), 11.57 (1H, br s)

### Reference Example 41

### 1-(3,4-Dimethoxyphenyl)-5-methyl-2-phenyl-1H-pyrrole-3-carboxylic acid

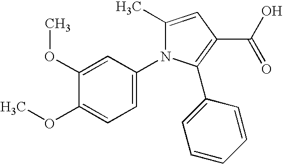

¹H-NMR (DMSO-d₆) δ 2.03 (3H, s), 3.58 (3H, s), 3.71 (3H, s), 6.39 (1H, s), 6.60-6.75 (2H, m), 6.80-6.91 (1H, m), 7.17 (5H, s), 11.53 (1H, br s)

### Reference Example 42

### 1-[2-(Benzyloxy)phenyl]-5-methyl-2-phenyl-1H-pyrrole-3-carboxylic acid

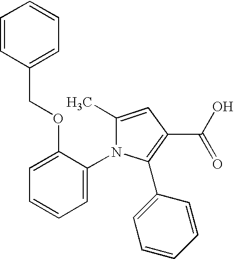

¹H-NMR (CDCl₃) δ 2.00 (3H, d), 4.91-5.05 (2H, m), 6,58 (1H, s), 6.80-6.89 (2H, m), 7.01 (1H, dd), 7.06-7.37 (11H, m)

### Reference Example 43

### 1-(2,3-Dimethoxyphenyl)-5-methyl-2-phenyl-1H-pyrrole-3-carboxylic acid

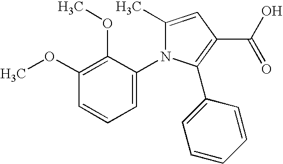

¹H-NMR (CDCl₃) δ 2.03 (3H, s), 3.57 (3H, s), 3.81 (3H, s), 3.81 (3H, s), 6.45-6.79 (2H, m), 6.77-7.03 (2H, m), 7.12-7.31 (5H, m)

### Reference Example 44

### 1-(2,2-Difluoro-1,3-benzodioxol-4-yl)-5-methyl-2-phenyl-1H-pyrrole-3-carboxylic acid

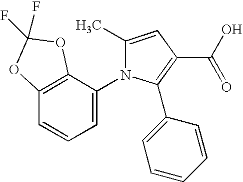

¹H-NMR (CDCl₃) δ 2.11 (3H, s), 6.60 (1H, s), 6.75 (1H, dd), 6.90-7.03 (2H, m), 7.18 (5H, s)

### Reference Example 45

### 1-(2-{[(tert-Butoxycarbonyl)amino]methyl}phenyl)-5-methyl-2-phenyl-1H-pyrrole-3-carboxylic acid

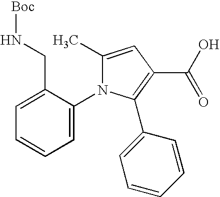

¹H-NMR (CDCl₃) δ 1.87 (9H, br s), 1.97 (3H, s), 3.19-3.45 (2H, m), 3.49 (1H, s), 6.61 (1H, s), 7.10-7.39 (9H, m)

### Reference Example 46

### Ethyl2-(2-thienylcarbonyl)-4-oxopentanoate

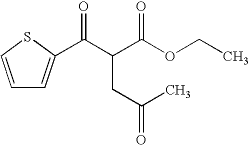

Chloroacetone (2.54 g) was added to a suspension of ethyl 3-oxo-3-(2-thienyl)propanoate (4.96 g), potassium carbonate (6.91 g), potassium iodide (830 mg) and acetone (50 ml) at room temperature. The reaction mixture was heated under reflux for 2 hr, and then the reaction mixture was concentrated in vacuo, diluted with water and extracted with ethyl acetate. The extract was washed successively with water and brine and dried over anhydrous magnesium sulfate, and then the solvent was evaporated in vacuo. The residue was subjected to silica gel column chromatography, and the fraction eluted with ethyl acetate-hexane (1:2) was concentrated in vacuo to give the desired product (6.15 g) as an oil.

¹H-NMR (CDCl₃) δ 1.19 (3H, t), 2.23 (3H, s), 3.19 (2H, d), 4.16 (2H, q), 4.73 (1H, t), 7.16 (1H, t), 7.71 (1H, d), 7.90 (1H, d)

### Reference Example 47

### 5-Methyl-1-phenyl-2-(2-thienyl)-1H-pyrrole-3-carboxylic acid

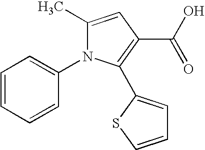

A solution of ethyl2-(2-thienylcarbonyl)-4-oxopentanoate (1.27 g), aniline (466 mg), p-toluenesulfonic acid hydrate (48 mg) and ethanol (25 ml) was heated under reflux for 15 hr. The reaction mixture was then poured into water and extracted with ethyl acetate. The extract was washed successively with 0.1 N hydrochloric acid, a saturated aqueous sodium bicarbonate solution, water and brine and dried over anhydrous magnesium sulfate, and then the solvent was evaporated in vacuo. The residue was subjected to silica gel column chromatography, and the fraction eluted with ethyl acetate-hexane (1:3) was concentrated in vacuo. 500 mg of the resulting crystals (520 mg) was dissolved in ethanol (5 ml), potassium hydroxide (270 mg) was added thereto, and the mixture was heated under reflux for 15 hr. The reaction mixture was poured into water, weakly acidified (pH 3) with concentrated hydrochloric acid, and then extracted with ethyl acetate-THF (2:1). The extract was washed successively with water and brine and dried over anhydrous magnesium sulfate. Then, the solvent was evaporated in vacuo, and the crystals were collected by filtration to give the desired product (400 mg).

¹H NMR (DMSO-d₆) δ 1.98 (3H, s), 6.43 (1H, s), 6.84-6.90 (2H, m), 7.18-7.21 (2H, m), 7.36-7.42 (4H, m), 11.74 (1H, br s)

In the same manner as in Reference Example 47, the following compounds (Reference Examples 48 to 49) were obtained.

### Reference Example 48

### 1-(2,3-Dimethoxyphenyl)-5-methyl-2-(2-thienyl)-1H-pyrrole-3-carboxylic acid

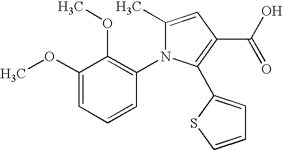

¹H-NMR (DMSO-d₆) δ 1.92 (3H, s), 3.50 (3H, s), 3.81 (3H, s), 6.42 (1H, s), 6.79 (1H, dd), 6.87 (1H, dd), 6.97 (1H, dd), 7.03-7.12 (2H, m), 7.41 (1H, dd), 11.71 (1H, br s)

### Reference Example 49

### 5-Methyl-1-(3-morpholinophenyl)-2-(2-thienyl)-1H-pyrrole-3-carboxylic acid

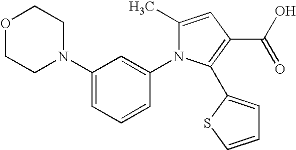

¹H-NMR (DMSO-d₆) δ 2.01 (3H, s), 3.02-3.05 (4H, m), 3.67-3.70 (4H, m), 6.40 (1H, s), 6.59 (1H, dd), 6.72 (1H, t), 6.86-6.92 (3H, m), 7.21 (1H, t), 7.42 (1H, dd), 11.70 (1H, br s)

### Reference Example 50

### 1-(3-Methoxypropyl)-4,5-diphenyl-1H-pyrrole-3-carboxylic acid

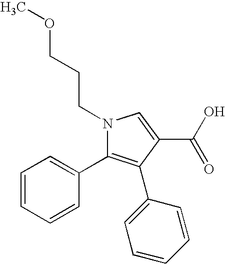

Methyl1-(3-methoxypropyl)-4,5-diphenyl-1H-pyrrole-3-carboxylate (155 mg) was suspended in ethanol (2 ml) and THF (2 ml). A 10% aqueous lithium hydroxide solution (4 ml) was added thereto, and the suspension was heated under reflux for 24 hr. The reaction mixture was concentrated in vacuo, weakly acidified (pH 3) by adding 2 N hydrochloric acid to the remaining aqueous solution, and then extracted with ethyl acetate. The extract was washed with brine and dried over anhydrous sodium sulfate. Then, the solvent was evaporated in vacuo, and the crystals were collected by filtration to give the desired product (120 mg).

¹H-NMR (CDCl₃) δ 1.72-1.86 (2H, m), 3.18-3,29 (5H, m), 3.96 (2H, t), 7.07-7.20 (7H, m), 7.22-7.34 (3H, m), 7.52-7.59 (1H, m)

In the same manner as in Reference Example 50, the following compounds (Reference Examples 51 to 52) were obtained.

### Reference Example 51

### 5-Acetyl-1,2-diphenyl-1H-pyrrole-3-carboxylic acid

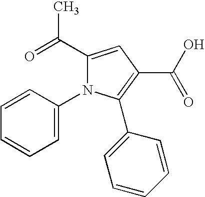

¹H-NMR (CDCl₃) δ 2.44 (3H, s), 6.89-7.38 (10H, m), 7.63 (1H, s)

### Reference Example 52

### 4-Acetyl-1,2-diphenyl-1H-pyrrole-3-carboxylic acid

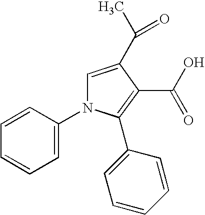

¹H-NMR (CDCl₃) δ 2.65 (3H, s), 6.93-7.46 (10H, m), 7.70 (1H, s), 13.80 (1H, br s)

### Reference Example 53

### Benzyl3-[4-(2-ethoxy-2-oxoethoxy)phenyl]-3-oxopropanoate

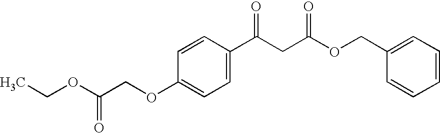

To a solution of 4-(2-ethoxy-2-oxoethoxy)benzoic acid (9.11 g) in THF (80 ml) was added CDI (7.91 g), and the mixture was stirred at room temperature for 1 hr. Then, potassium monobenzyl malonate (9.91 g) and anhydrous magnesium chloride (4.06 g) were further added thereto, and the mixture was heated under reflux for 2 hr. The reaction mixture was poured into ice-water, weakly acidified (pH 3) with concentrated hydrochloric acid, and then extracted with ethyl acetate. The extract was washed successively with water and brine and dried over anhydrous magnesium sulfate, and then the solvent was evaporated in vacuo. The residue was subjected to silica gel column chromatography, and the fraction eluted with ethyl acetate-hexane (1:4 to 1:2) was concentrated-in vacuo to give the desired product (12.92 g) as an oil.

¹H-NMR (CDCl₃) δ 1.30 (3H, t), 3.99 (2H, s), 4.28 (2H, q), 4.68 (2H, s), 5.18 (2H, s), 6.92 (2H, d), 7.25-7.39 (5H, m), 7.90 (2H, d)

### Reference Example 54

### Benzyl2-[4-(2-ethoxy-2-oxoethoxy)benzoyl]-4-oxopentanoate

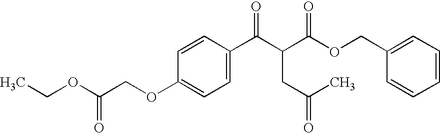

Chloroacetone (3.69 g) was added to a suspension of benzyl3-[4-(2-ethoxy-2-oxoethoxy)phenyl]-3-oxopropanoate (12.91 g), potassium carbonate (10.01 g), potassium iodide (1.20 g) and acetone (50 ml) at room temperature. The reaction mixture was heated under reflux for 2 hr, and the mixture was poured into water and extracted with ethyl acetate. The extract was washed successively with water and brine and dried over anhydrous magnesium sulfate, and then the solvent was evaporated in vacuo. The residue was subjected to silica gel column chromatography, and the fraction eluted with ethyl acetate-hexane (1:2 to 1:1.5) was concentrated in vacuo to give the desired product (12.16 g) as an oil.

¹H-NMR (CDCl₃) δ 1.31 (3H, t), 2.21 (3H, s), 3.18 (2H, ddd), 4.28 (2H, q), 4.67 (2H, s), 4.90 (1H, t), 5.09 (2H, s), 6.91 (2H, d), 7.15-7.18 (2H, m), 7.25-7.29 (3H, m), 7.98 (2H, d)

In the same manner as in Reference Example 54, the following compound (Reference Example 55) was obtained.

### Reference Example 55

### Benzyl2-benzoyl-4-oxopentanoate

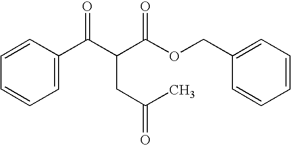

¹H-NMR (CDCl₃) δ 2.21 (3H, s), 3.20 (2H, ddd), 4.95 (1H, dd), 5.09 (2H, s), 7.12-7.16 (2H, m), 7.24-7.28 (3H, m), 7.41-7.47 (2H, m), 7.54-7.59 (1H, m), 7.97-8.00 (2H, m)

### Reference Example 56

### Ethyl3-(3-amino-4,5-dimethoxyphenyl)propanoate

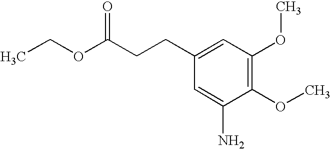

Ethyl(2E)-3-(3,4-dimethoxy-5-nitrophenyl)acrylate (4.84 g) was dissolved in methanol (140 ml). 10% Palladium on carbon (containing 50% water, 2.4 g) was added thereto, and the mixture was subjected to catalytic hydrogenation at room temperature and atmospheric pressure for 4.5 hr. The catalyst was filtered off, and the filtrate was concentrated in vacuo to give the desired product (4.16 g) as an oil.

¹H-NMR (CDCl₃) δ 1.25 (3H, t), 2.57 (2H, t), 2.80 (2H, t), 3.79 (3H, s), 3.80 (2H, s), 3.82 (3H, s), 4.13 (2H, q), 6.17 (1H, d), 6.22 (1H, d)

MS (ESI+, m/e) 254 (M+1)

### Reference Example 57

### Benzyl1-(2,3-dimethoxyphenyl)-2-(4-(2-ethoxy-2-oxoethoxy)phenyl]-5-methyl-1H-pyrrole-3-carboxylate

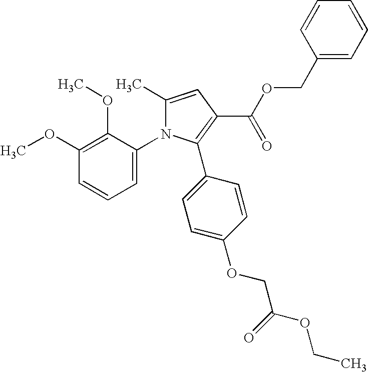

A solution of benzyl2-[4-(2-ethoxy-2-oxoethoxy)benzoyl]-4-oxopentanoate (5.00 g), 2,3-dimethoxyaniline (2.23 g), p-toluene sulfonic acid hydrate (184 mg) and ethanol (60 ml) was heated under reflux for 18 hr. Then, the mixture was poured into water and extracted with ethyl acetate. The extract was washed successively with 0.1 N hydrochloric acid, a saturated aqueous sodium bicarbonate solution, water and brine and dried over anhydrous magnesium sulfate, and then the solvent was evaporated in vacuo. The residue was subjected to silica gel column chromatography, and the fraction eluted with ethyl acetate-hexane (1:1.5) was concentrated in vacuo to give the desired product (1.80 g) as an oil.

¹H-NMR (CDCl₃) δ 1.27 (3H, t), 2.02 (3H, s), 3.56 (3H, s), 3.82 (3H, s), 4.25 (2H, q), 4.52 (2H, s), 5.16 (2H, s), 6.57 (1H, s), 6.65-6.70 (2H, m), 6.85 (1H, dd), 6.94 (1H, t), 7.15-7.30 (8H, m)

MS (ESI+, m/e) 530 (M+1)

In the same manner as in Reference Example 57, the following compounds (Reference Examples 58 to 60) were obtained.

### Reference Example 58

### Benzyl1-(2,3-dimethoxy-5-(methoxycarbonyl)phenyl]-5-methyl-2-phenyl-1H-pyrrole-3-carboxylate

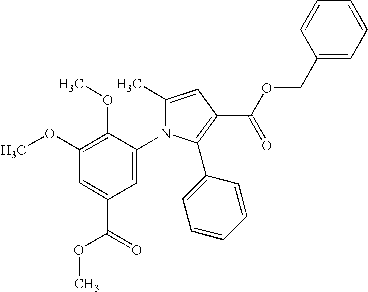

¹H-NMR (CDCl₃) δ 2.02 (3H, s), 3.61 (3H, s), 3.85 (3H, s), 3.88 (3H, s), 5.15 (2H, s), 6.59 (1H, s), 7.15-7.25 (10H, m), 7.45 (1H, d), 7.52 (1H, d)

### Reference Example 59

### Benzyl1-[5-(3-ethoxy-3-oxopropyl)-2,3-dimethoxyphenyl]-5-methyl-2phenyl-1H-pyrrole-3-carboxylate

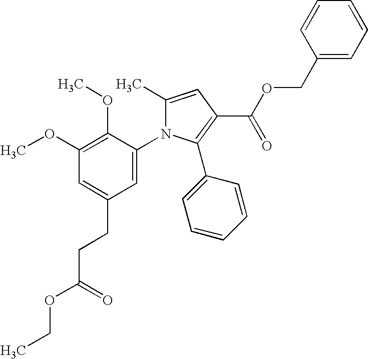

¹H-NMR (CDCl₃) δ 1.23 (3H, t), 2.03 (3H, s), 2.44 (2H, t), 2.80 (2H, t), 3.54 (3H, s), 3.79 (3H, s), 4.11 (2H, q), 5.15 (2H, s), 6.48 (1H, d), 6.58 (1H, s), 6.68 (1H, d), 7.15-7.28 (10H, m)

### Reference Example 60

### Benzyl1-[3-(benzyloxy)phenyl]-5-methyl-2-phenyl-1H-pyrrole-3-carboxylate

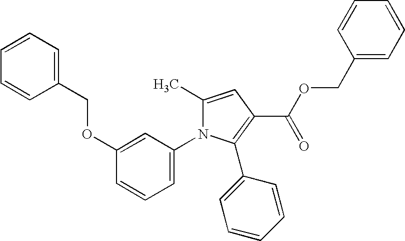

¹H-NMR (CDCl₃) δ 2.17 (3H, s), 4.91 (2H, s), 5.15 (2H, s), 6.54-6.67 (3H, m), 6.83-6.91 (1H, m), 7.09-7.21 (7H, m), 7.23-7.49 (9H, m)

### Reference Example 61

### 1-(2,3-Dimethoxyphenyl)-2-[4-(2-ethoxy-2-oxoethoxy)phenyl]-5-methyl-1H-pyrrole-3-carboxylic acid

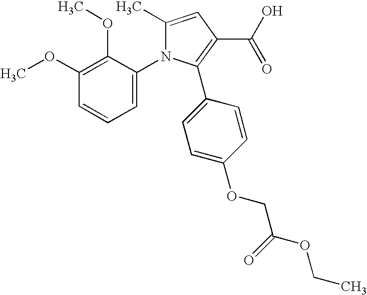

Benzyl1-(2,3-dimethoxyphenyl)-2-[4-(2-ethoxy-2-oxoethoxy)phenyl]-5-methyl-1H-pyrrole-3-carboxylate (1.79 g) was dissolved in ethanol-THF (1:1, 60 ml). 10% Palladium on carbon (containing 50% water, 900 mg) was added thereto, and the mixture was subjected to catalytic hydrogenation at room temperature and atmospheric pressure for 3 hr. The catalyst was filtered off, the filtrate was concentrated in vacuo, and the crystals were collected by filtration to give the desired product (1.24 g). A portion thereof was recrystallized from THF-ethyl acetate and taken as a sample for analysis.

¹H-NMR (DMSO-d₆) δ 1.16 (3H, t), 1.91 (3H, s), 3.45 (3H, s), 3.76 (3H, s), 4.13 (2H, q), 4.69 (2H, s), 6.36 (1H, s), 6.70 (2H, d), 6.78-6.82 (1H, m); 7.01-7.03 (2H, m), 7.08 (2H, d), 11.43 (1H, br s)

MS (ESI+, m/e) 440 (M+1)

In the same manner as in Reference Example 61, the following compounds (Reference Examples 62 to 63) were obtained.

### Reference Example 62

### 1-[2,3-Dimethoxy-5-(methoxycarbonyl)phenyl]-5-methyl-2-phenyl-1H-pyrrole-3-carboxylic acid

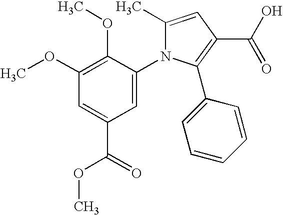

¹H-NMR (DMSO-d₆) δ 1.94 (3H, s), 3.57 (3H, s), 3.81 (3H, s), 3.83 (3H, s), 6.42 (1H, s), 7.16 (5H, s), 7.36 (1H, d), 7.51 (1H, d), 11.61 (1H, br s)

MS (ESI+, m/e) 396 (M+1)

### Reference Example 63

### 1-[5-(3-Ethoxy-3-oxopropyl)-2,3-dimethoxyphenyl]-5-methyl-2-phenyl-1H-pyrrole-3-carboxylic acid

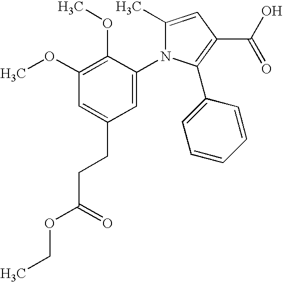

¹H-NMR (DMSO-d₆) δ 1.15 (3H, t), 1.92 (3H, s), 2.54 (2H, t), 2.75 (2H, t), 3.38 (3H, s), 3.73 (3H, s), 4.03 (2H, q), 6.38 (1H, s), 6.69 (1H, d), 6.90 (1H, d), 7.16 (5H, s), 11.50 (1H, 5)

MS (ESI+, m/e) 438 (M+1)

### Reference Example 64

### 1-(3-Hydroxyphenyl)-5-methyl-2-phenyl-1H-pyrrole-3-carboxylic acid

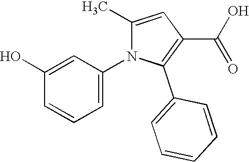

Benzyl1-[3-(benzyloxy)phenyl]-5-methyl-2-phenyl-1H-pyrrole-3-carboxylate (4.3 g) was dissolved in methanol (100 ml). 10% Palladium on carbon (containing 50% water, 1.0 g) was added thereto, and the mixture was subjected to catalytic hydrogenation at room temperature and atmospheric pressure for 12 hr. The catalyst was filtered off, and the filtrate was concentrated in vacuo to give the desired product (2.7 g) as an amorphous solid.

¹H-NMR (DMSO-d₆) δ 2.00 (3H, s), 3.33 (1H, br s), 6.37-6.40 (1H, m), 6.43-6.47 (1H, m), 6.52-6.58 (1H, m), 6.66-6.72 (1H, m), 7.07-7.34 (6H, m), 10.60 (1H, br s)

### Reference Example 65

### 1-tert-Butyl-5-(4-fluorophenyl)-1H-pyrazole-4-carboxylic acid

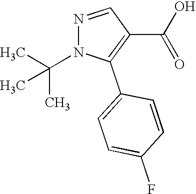

A solution of ethyl (p-fluorobenzoyl)acetate (0.75 g) and N,N-dimethylformamide dimethylacetal (0.57 ml) in toluene (10 ml) was heated under reflux for 3 hr under nitrogen atmosphere. The reaction mixture was concentrated in vacuo, and then the residue was dissolved in ethanol (10 ml). Triethylamine (0.52 ml) and tert-butylhydrazine (0.49 g) were added thereto, and the mixture was stirred at 80° C. for 2 hr. Then, the reaction mixture was concentrated in vacuo, and the residue was extracted with ethyl acetate, washed successively with water and brine and dried over anhydrous magnesium sulfate. The solvent was evaporated in vacuo, the residue was subjected to silica gel column chromatography, and the fraction eluted with ethyl acetate-hexane (18:82 to 35:65) was concentrated in vacuo to give an oil (0.31 g). The resulting oil was mixed with a 1 N aqueous sodium hydroxide solution (2 ml) and ethanol (3 ml), and the mixture was stirred at 50° C. for 3 hr. The reaction mixture was concentrated in vacuo, and the residue was dissolved in water, washed with diethyl ether, then acidified with 1 N hydrochloric acid and extracted with ethyl acetate. The extract was washed successively with water and brine and dried over anhydrous magnesium sulfate, and then concentrated in vacuo. The residue was recrystallized from ethyl acetate-hexane to give the desired product (0.21 g).

¹H-NMR (CDCl₃) δ 1.45 (9H, s), 7.10-7.20 (2H, m), 7.24-7.35 (2H, m), 7.99 (1H, s)

In the same manner as in Reference Example 65, the following compounds (Reference Examples 66 to 68) were obtained.

### Reference Example 66

### 1-tert-Butyl-5-cyclopropyl-1H-pyrazole-4-carboxylic acid

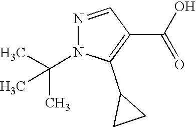

¹H-NMR (CDCl₃) δ 0.95-1.04 (2H, m), 1.12-1.21 (2H, m), 1.75 (9H, s), 1.83-1.99 (1H, m), 7.87 (1H, s)

### Reference Example 67

### 5-Cyclopropyl-1-phenyl-1H-pyrazole-4-carboxylic acid

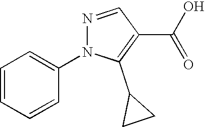

¹H-NMR (CDCl₃) δ 0.69 (2H, ddd), 0.92-0.99 (2H, m), 1.96-2.06 (1H, m), 7.42-7.56 (5H, m), 8.10-8.13 (1H, m)

### Reference Example 68

### 1-Phenyl-5-(2-thienyl)-1H-pyrazole-4-carboxylic acid

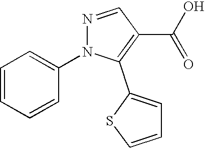

¹H-NMR (DMSO-d₆) δ 7.05 (1H, dd), 7.18 (1H, dd), 7.28-7.31 (2H, m), 7.39-7.44 (3H, m), 7.69 (1H, dd), 8.15 (1H, s), 12.53 (1 H, br s)

### Reference Example 69

### 1-Benzyl-5-phenyl-1H-pyrazole-4-carboxylic acid

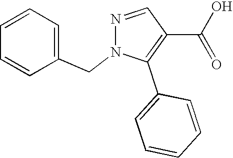

To a solution of ethyl1-benzyl-5-phenyl-1H-pyrazole-4-carboxylate (2.12 g) in ethanol (30 ml) and tetrahydrofuran (30 ml) were added a 1 N aqueous sodium hydroxide solution (20 ml) and a 1 N aqueous lithium hydroxide solution (6 ml), and the mixture was heated under reflux for 4 hr. The reaction mixture was concentrated in vacuo, and water was added to the residue. The reaction mixture was washed with diethyl ether, then acidified with 1 N hydrochloric acid and extracted with ethyl acetate. The extract was washed with brine and dried over anhydrous sodium sulfate, and the solvent was evaporated in vacuo. The residue was recrystallized from ethyl acetate-hexane to give the desired product (1.87 g).

¹H-NMR (DMSO-d₆) δ 5.16 (2H, s), 6.97-7.03 (2H, m), 7.25-7.31 (5H, m), 7.38-7.49 (3H, m), 8.09 (1H, s)

### Reference Example 70

### 5-(4-Fluorophenyl)-1-(pyridin-2-yl)-1H-pyrazole-4-carboxylic acid

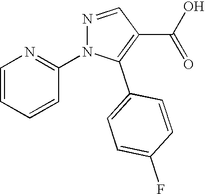

A solution of ethyl (p-fluorobenzoyl)acetate (5.0 g) and N,N-dimethylformamide dimethylacetal (3.48 ml) in toluene (50 ml) was heated under reflux for 15 hr under nitrogen atmosphere. The reaction mixture was concentrated in vacuo, and then the residue was dissolved in ethanol (50 ml) and 2-hydrazinopyridine (2.6 g) was added thereto. After stirring at 65° C. for 4 hr, the reaction mixture was concentrated in vacuo, and the residue was extracted with ethyl acetate, washed successively with water and brine and dried over anhydrous magnesium sulfate. The solvent was evaporated in vacuo, the residue was subjected to silica gel column chromatography, and the fraction eluted with ethyl acetate-hexane (1:4 to 1:3) was concentrated in vacuo. A mixture of a portion of the residue (5.02 g), a 2 N aqueous sodium hydroxide solution (16 ml) and ethanol (50 ml) was stirred at 45° C. for 10 hr. The reaction mixture was cooled to room temperature, 2 N hydrochloric acid and water were added thereto and stirred at room temperature for 30 min. The crystals were collected by filtration and washed with water to give the desired product (4.21 g).

¹H-NMR (CDCl₃) δ 6.97-7.05 (2H, m), 7.20-7.32 (3H, m), 7.35-7.40 (1H, m), 7.69-7.80 (1H, m), 8.23 (1H, s), 8.30-8.35 (1H, m)

### Reference Example 71

### 5-(4-Fluorophenyl)-1-(2-methoxyphenyl)-1H-pyrazole-4-carboxylic acid

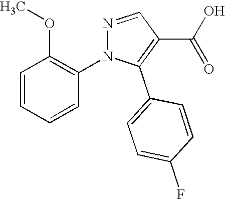

A solution of ethyl (p-fluorobenzoyl)acetate (3.07 g) and N,N-dimethylformamide dimethylacetal (2.13 ml) in toluene (30 ml) was heated under reflux for 15 hr under nitrogen atmosphere. The reaction mixture was concentrated in vacuo, and then the residue was dissolved in ethanol (30 ml). (2-Methoxyphenyl)hydrazine (2.55 g) and triethylamine (2.24 ml) were added thereto. After stirring at 80° C. for 2 hr, the reaction mixture was concentrated in vacuo, and the residue was extracted with ethyl acetate, washed successively with water and brine and dried over magnesium sulfate. The solvent was evaporated in vacuo, the residue was subjected to silica gel column chromatography, and the fraction eluted with ethyl acetate-hexane (16:84 to 20:80) was concentrated in vacuo to give ethyl5-(4-fluorophenyl)-1-(2-methoxyphenyl)-1H-pyrazole-4-carboxylate. A mixture of a portion of the resulting compound (1.0 g), a 2 N aqueous sodium hydroxide solution (2.5 ml) and ethanol (10 ml) was stirred at room temperature for 15 hr. The reaction mixture was cooled to room temperature, 2 N hydrochloric acid and water were added thereto and stirred at room temperature for 30 min. The crystals were collected by filtration and washed with water to give the desired product (0.62 g).

¹H-NMR (CDCl₃) δ 3.54 (3H, s), 6.82 (1H, d), 6.90-7.02 (3H, m), 7.20-7_{—}40 (3H, m), 8.24 (1H, s)

In the same manner as in Reference Example 71, the following compounds (Reference Examples 72 to 74) were obtained.

### Reference Example 72

### 5-Phenyl-1-(2-methoxyphenyl)-1H-pyrazole-4-carboxylic acid

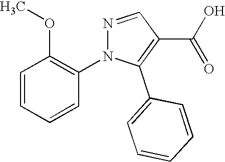

MS (ESI+, m/e) 295 (M+1)

### Reference Example 73

### 5-Phenyl-1-(3-methoxyphenyl)-1H-pyrazole-4-carboxylic acid

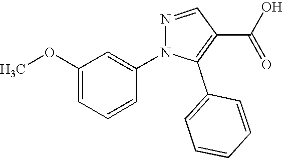

MS (ESI+, m/e) 295 (M+1)

### Reference Example 74

### 5-Phenyl-1-(4-methoxyphenyl)-1H-pyrazole-4-carboxylic acid

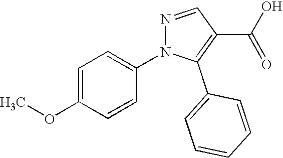

MS (ESI+, m/e) 295 (M+1)

### Reference Example 75

### 1-[2-(Benzyloxy)phenyl]-5-(4-fluorophenyl)-1H-pyrazole-4-carboxylic acid

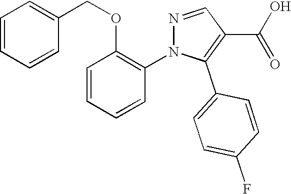

To a solution of ethyl5-(4-fluorophenyl)-1-(2-methoxyphenyl)-1H-pyrazole-4-carboxylate (2.85 g) obtained in Reference Example 71 in dichloromethane was added dropwise boron tribromide (a 1 M dichloromethane solution, 42 ml) at −78° C., and the mixture was stirred at room temperature overnight. The reaction mixture was poured into ice-water, and the precipitated crystals (1.09 g) were collected by filtration. The filtrate was extracted with dichloromethane, washed successively with water and brine and dried over anhydrous magnesium sulfate, and then concentrated in vacuo to give crude crystals (2.68 g). The resulting crystals were combined and dissolved in DMF (35 ml). Benzyl bromide (2 ml) and potassium carbonate (4.65 g) were added thereto and stirred at 50° C. for 7 hr. Water was added thereto, and the reaction mixture was extracted with ethyl acetate, dried over anhydrous magnesium sulfate, and then concentrated in vacuo to give an oil (4.15 g). The resulting oil was dissolved in ethanol (50 ml), a 2 N aqueous sodium hydroxide solution (10 ml) was added thereto and stirred at 50° C. for 11 hr. 1 N Hydrochloric acid was added thereto, and the reaction mixture was then concentrated in vacuo and diluted with ethyl acetate. The organic layer was washed successively with water and brine, dried over anhydrous magnesium sulfate and concentrated in vacuo. The residue was recrystallized from toluene-hexane to give the desired product (3.13 g).

¹H-NMR (CDCl₃) δ 4.84 (2H, s), 6.80-7.40 (13H, m), 8.25 (1H, s)

### Reference Example 76

### 5-Cyclohexyl-1-phenyl-1H-pyrazole-4-carboxylic acid

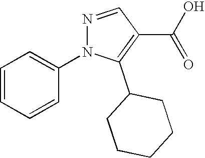

Methyl5-cyclohexyl-1-phenyl-1H-pyrazole-4-carboxylate (4.90 g) was suspended in ethanol (50 ml). A 3 N aqueous sodium hydroxide solution (34 ml) was added thereto, and the suspension was heated under reflux overnight. The reaction mixture was poured into water, weakly acidified (pH 3) with 6 N hydrochloric acid, and then extracted with ethyl acetate. The extract was washed with brine and dried over anhydrous magnesium sulfate. Then, the solvent was evaporated in vacuo, and the crystals were collected by filtration to give the desired product (4.30 g).

¹H-NMR (DMSO-d₆) δ 1.03-1.18 (3H, m), 1.57 (3H, s), 1.71 (2H, s), 2.07 (2H, d), 2.80 (1H, d), 7.39-7.47 (2H, m), 7.55-7.63 (3H, m), 7.94 (1H, s), 12.35 (1H, s)

### Reference Example 77

### 1-[4-(Benzyloxy)phenyl]-5-phenyl-1H-1,2,3-triazole-4-carboxylic acid

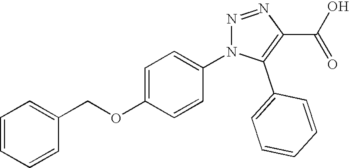

1-Azide-4-(benzyloxy)benzene (4.5 g) was dissolved in methanol (200 ml), and ethyl benzoylacetate (5.77 g) was added thereto at 0° C. Next, a 28% solution of sodium methoxide in methanol (5.79 g) was added dropwise thereto, and the mixture was stirred at 60° C. for 3 hr. A 1 N aqueous sodium hydroxide solution (40 ml) was added thereto, stirred at 50° C. for 1 hr, and then the precipitated crystals were collected by filtration. The filtrate was suspended in 1 N hydrochloric acid (50 ml) and the reaction mixture was stirred at room temperature for 30 min. Then, the precipitated crystals were collected by filtration, washed with water and dried over in vacuo to give the desired product (2.4 g).

¹H-NMR (DMSO-d₆) δ 5.10 (2H, s), 7.05-7.09 (2H, m), 7.28-7.46 (12H, m), 13.03 (1H, s)

In the same manner as in Reference Example 77, the following compounds (Reference Examples 78 to 79) were obtained.

### Reference Example 78

### 1-[3-(Benzyloxy)phenyl]-5-phenyl-1H-1,2,3-triazole-4-carboxylic acid

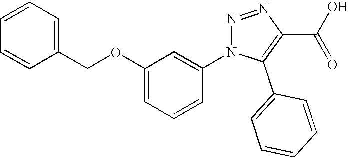

¹H-NMR (DMSO-d₆) δ 5.03 (2H, s), 6.91 (1H, d), 7.08-7.13 (2H, m), 7.32-7.42 (11H, m), 13.06 (1H, s)

### Reference Example 79

### 1-[2-(Benzyloxy)phenyl]-5-phenyl-1H-1,2,3-triazole-4-carboxylic acid

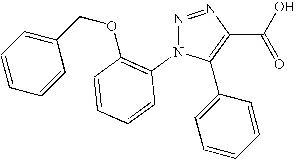

¹H-NMR (DMSO-d₆) δ 4.99 (2H, s), 7.04-7.15 (4H, m), 7.22-7.40 (8H, m), 7.46 (1H, td), 7.57 (1H, dd), 13.06 (1H, s)

### Reference Example 80

### 1-Phenyl-5-(pyridin-2-yl)-1H-1,2,3-triazole-4-carboxylic acid

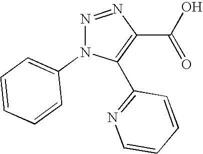

Phenyl azide (596 mg) was dissolved in methanol (50 ml), and ethyl3-oxo-3-(pyridin-2-yl)propanoate (1.06 g) was added thereto at room temperature. Next, a 28% solution of sodium methoxide in methanol (1.06 g) was added dropwise thereto, and the mixture was stirred at 60° C. for 3 hr. A 1 N aqueous sodium hydroxide solution (5 ml) was added thereto and stirred at 50° C. for 1 hr. The reaction mixture was neutralized with 1 N hydrochloric acid. The liberated oil was washed with ethyl acetate (10 ml×2) and removed. The aqueous layer was concentrated and dried, and the residue was then subjected to silica gel column chromatography. The fraction eluted with ethyl acetate-methanol (10:1 to 5:1) was concentrated in vacuo, and the crystals were collected by filtration to give the desired product (150 mg).

¹H-NMR (DMSO-d₆) δ 7.24-7.43 (5H, m), 7.52 (1H, s), 7.83-7.89 (2H, m), 8.03 (1H, t), 8.40 (1H, d)

### Reference Example 81

### 5-Cyclopropyl-1-phenyl-1H-1,2,3-triazole-4-carboxylic acid

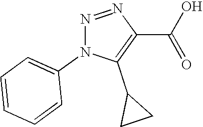

Phenyl azide (1.2 g) was dissolved in methanol (100 ml), and ethyl3-cyclopropyl-3-oxopropanoate (2.13 g) was added thereto at room temperature. Next, a 28% solution of sodium methoxide in methanol (2.9 g) was added dropwise thereto, and the reaction mixture was stirred at 60° C. for 3 hr. A 1 N aqueous sodium hydroxide solution (20 ml) was added thereto, stirred at 50° C. for 1 hr, and then weakly acidified (pH 2 to 3) with 1 N hydrochloric acid. The liberated oil was extracted with chloroform, and the extract was washed with brine and dried over anhydrous magnesium sulfate. The solvent was evaporated in vacuo, and the crystals were collected by filtration to give the desired product (1.7 g).

¹H-NMR (DMSO-d₆) δ 0.61-0.67 (2H, m), 0.84-0.90 (2H, m), 2.05-2.15 (1H, m), 7.62-7.69 (5H, m), 13.03 (1H, s)

### Reference Example 82

### 5-(2-Thienyl)-1-(4-{[(2,2,2-trifluoroethyl)amino]carbonyl}phenyl)-1H-1,2,3-triazole-4-carboxylic acid

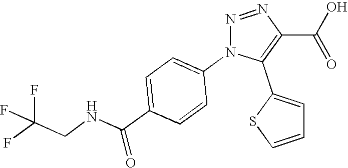

A solution of 4-azidobenzoic acid (2.5 g), 2,2,2-trifluoroethylamine (1.8 g), WSC.HCl (4.4 g), HOBt (1.1 g), triethylamine (2.5 ml) and DMF (30 ml) was stirred at room temperature for 15 hr, and then a 10% aqueous sodium bicarbonate solution (50 ml) was added thereto. The precipitated crystals were collected by filtration, washed with water and then dried in vacuo. To a portion thereof (335 mg) was added a solution of ethyl3-oxo-3-(2-thienyl)propanoate (270 mg), a 20% solution of sodium ethoxide in ethanol (700 mg) and ethanol (10 ml). The mixture was stirred at 60° C. for 18 hr, and then neutralized with 1 N hydrochloric acid. The precipitated crystals were collected by filtration, washed with water and dried in vacuo to give the desired product (518 mg).

¹H-NMR (DMSO-d₆) δ 4.05-4.17 (2H, m), 7.11 (1H, dd), 7.33 (1H, dd), 7.60 (2H, d), 7.78 (1H, dd), 8.00 (2H, d), 9.29 (1H, t), 13.31 (1H, br s)

In the same manner as in Reference Example 82, the following compound (Reference Example 83) was obtained.

### Reference Example 83

### 5-(1,3-Thiazol-2-yl)-1-(4-{[(2,2,2-trifluoroethyl)amino]carbonyl}phenyl)-1H-1,2,3-triazole-4-carboxylic acid

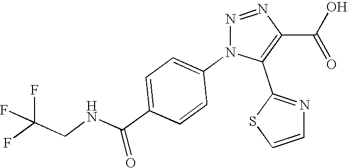

¹H-NMR (DMSO-d₆) δ 4.05-4.17 (2H, m), 7.58 (2H, d), 7.92 (1H, d), 7.98 (2H, d), 8.07 (1H, d), 9.28 (1H, t), 13.73 (1H, br s)

### Reference Example 84

### 1-(3-Morpholinophenyl)-5-phenyl-1H-imidazole-4-carboxylic acid

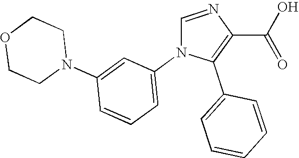

Methyl1-(3-morpholinophenyl)-5-phenyl-1H-imidazole-4-carboxylate (883 mg) was suspended in methanol (10 ml), a 4 N aqueous sodium hydroxide solution (14 ml) was added thereto, and the mixture was heated under reflux for 40 min. The reaction mixture was poured into water, and the reaction mixture was neutralized with 6 N hydrochloric acid, then saturated with sodium chloride and extracted with ethyl acetate-THF (2:1). The extract was washed with brine and dried over anhydrous magnesium sulfate. Then, the solvent was evaporated in vacuo, and the crystals were collected by filtration to give the desired product (644 mg). A portion thereof was recrystallized from THF-ethyl acetate and taken as a sample for analysis.

¹H-NMR (DMSO-d₆) δ 2.97-3.00 (4H, m), 3.60-3.67 (4H, m), 6.55 (1H, d), 6.71 (1H, s), 6.89 (1H, dd), 7.17 (1H, t), 7.23-7.30 (5H, m), 8.02 (1H, s), 12.11 (1H, br s)

MS (ESI+, m/e) 350 (M+1)

### Reference Example 85

### 1-(2,3-Dimethoxyphenyl)-5-phenyl-1H-imidazole-4-carboxylic acid

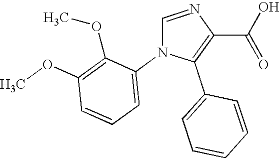

Methyl1-(2,3-dimethoxyphenyl)-5-phenyl-1H-imidazole-4-carboxylate (155 mg) was suspended in methanol (2 ml), a 4 N aqueous sodium hydroxide solution (3 ml) was added thereto, and the mixture was heated under reflux for 40 min. The reaction mixture was poured into water, and the reaction mixture was weakly acidified (pH 3) with 2 N hydrochloric acid, and then extracted with ethyl acetate. The extract was washed with brine and dried over anhydrous magnesium sulfate. Then, the solvent was evaporated in vacuo, and the crystals were collected by filtration to give the desired product (124 mg).

¹H-NMR (DMSO-d₆) δ 3.51 (3H, s), 3.79 (3H, s), 6.83 (1H, dd), 7.03 (1H, t), 7.10 (1H, dd), 7.21-7.26 (5H, m), 7.88 (1H, s), 12.11 (1H, br s)

### Reference Example 86

### 5-Phenyl-1-[(1S)-1-phenylethyl]-1H-imidazole⁻4-carboxylic acid

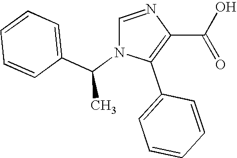

Methyl5-phenyl-1-[(1S)-1-phenylethyl]-1H-imidazole-4-carboxylate (1.18 g) was dissolved in methanol (22 ml), a 4 N aqueous sodium hydroxide solution (22 ml) was added thereto and stirred at 50° C. for 50 min. The reaction mixture was poured into water, weakly acidified (pH 3) with concentrated hydrochloric acid, and then extracted with ethyl acetate-THF (2:1). The extract was washed with brine and dried over anhydrous magnesium sulfate. Then, the solvent was evaporated in vacuo, and the crystals were collected by filtration to give the desired product (1.05 g). A portion thereof was recrystallized from THF-ethyl acetate and taken as a sample for analysis.

¹H-NMR (DMSO-d₆) δ 1.80 (3H, d), 5.10 (1H, q), 6.93-6.95 (2H, m), 7.16-7.19 (2H, m), 7.23-7.30 (3H, m), 7.35-7.44 (3H, m), 8.20 (1H, s), 11.98 (1H, br s)

MS (ESI+, m/e) 293 (M+1)

In the same manner as in Reference Example 86, the following compounds (Reference Examples 87 to 89) were obtained.

### Reference Example 87

### 5-Phenyl-1-[(1R)-1-phenylethyl]-1H-imidazole-4-carboxylic acid

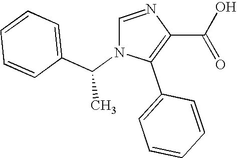

¹H-NMR (DMSO-d₆) δ 1.80 (3H, d), 5.09 (1H, q), 6.93-6.95 (2H, m), 7.16-7.19 (2H, m), 7.23-7.30 (3H, m), 7.34-7.43 (3H, m), 8.13 (1H, s), 11.96 (1H, br s)

MS (ESI+, m/e) 293 (M+1)

### Reference Example 88

### 1-[(1R)-2,3-Dihydro-1H-inden-1-yl]-5-phenyl-1H-imidazole-4-carboxylic acid

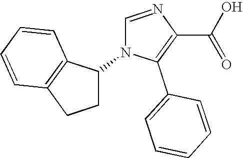

¹H-NMR (DMSO-d₆) δ 2.18-2.30 (1H, m), 2.47-2.58 (1H, m), 2.78-2.89 (1H, m), 2.96-3.06 (1H, m), 5.35 (1H, t), 7.02 (1H, d), 7.17-7.32 (3H, m), 7.41-7.51 (5H, m), 7.56 (1H, s), 11.94 (1H, br s)

MS (ESI+, m/e) 305 (M+1)

### Reference Example 89

### 5-Phenyl-1-[(1R)-1,2,3,4-tetrahydronaphthalen-1-yl]-1H-imidazole-4-carboxylic acid

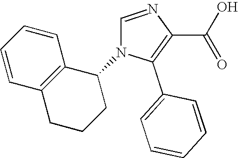

¹H-NMR (DMSO-d₆) δ 1.61-1.70 (1H, m), 1.84-1.88 (1H, m), 1.97-2.11 (2H, m), 2.69 (1H, dt), 2.84 (1H, ddd), 5.01 (1H, dd), 6.73 (1H, d), 7.09-7.22 (3H, m), 7.40-7.49 (5H, m), 7.57 (1H, s), 11.94 (1H, br s)

MS (ESI+, m/e) 319 (M+1)

### Reference Example 90

### 1-(2,3-Dihydro-1H-inden-2-yl)-5-phenyl-1H-imidazole-4-carboxylic acid

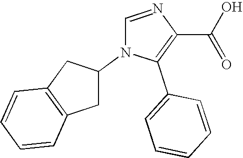

Methyl1-(2,3-dihydro-1H-inden-2-yl)-5-phenyl-1H-imidazole-4-carboxylate (1.27 g) was dissolved in methanol (23 ml), a 4 N aqueous sodium hydroxide solution (23 ml) was added, and the mixture was stirred at 50° C. for 50 min. The reaction mixture was poured into water, and the mixture was weakly acidified (pH 3) with concentrated hydrochloric acid, and extracted with ethyl acetate-THF (2:1). The extract was washed with brine, and dried over anhydrous magnesium sulfate, and the solvent was evaporated in vacuo. The crystals were collected by filtration to give the desired product (1.07 g). A part thereof was recrystallized from THF-ethyl acetate to give a sample for analysis.

¹H-NMR (DMSO-d₆) δ 3.23 (4H, d), 4.61 (1H, quintet), 7.16-7.24 (4H, m), 7.41-7.52 (5H, m), 7.80 (1H, s), 11.89 (1H, br s)

MS (ESI+, m/e) 305 (M+1)

### Reference Example 91

### (3R)-1,3Dibenzyl-1,4-diazepan

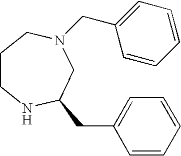

A mixture of (3R)-1,3-dibenzyl-1,4-diazepan-2,5-dione (4.22 g) and THF (125 ml) was ice-cooled, and lithium aluminum hydride (2.08 g) was added portionwise thereto. After stirring at room temperature for 30 min and at 60° C. for 16 hr, the mixture was cooled to −78° C., and ethanol-ethyl acetate (1:1, 14 ml) and a 1 N aqueous sodium hydroxide solution (28 ml) were successively added dropwise thereto. After the completion of the dropwise addition, the reaction mixture was stirred at room temperature for 40 min. The insolubles were filtered off and washed with ethyl acetate. The filtrate was washed with brine and dried over anhydrous magnesium sulfate, and then the solvent was evaporated in vacuo. The residue was subjected to basic silica gel column chromatography, and the fraction eluted with ethyl acetate-hexane (1:1) was concentrated in vacuo to give the desired product (3.42 g) as an oil.

¹H-NMR (CDCl₃) δ 1.66-1.75 (3H, m), 2.39-2.84 (7H, m), 2.93-3.08 (2H, m), 3.65 (2H, s), 7.11-7.35 (10H, m)

MS (ESI+, m/e) 281 (M+1)

### Reference Example 92

### EthylN-(tert-butoxycarbonyl)-3-(2-thienyl)-D-alanyl-N-benzylglycinate

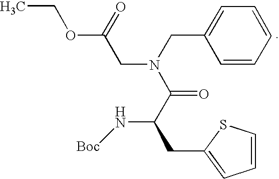

A solution of N-(tert-butoxycarbonyl)-3-(2-thienyl)-D-alanine (5.00 g), ethyl N-benzylglycinate (3.63 g), WSC.HCl (4.24 g), HOBt (2.74 g) and DMF (90 ml) was stirred at room temperature for 15 hr. Then, the reaction mixture was poured into water and extracted with ethyl acetate. The extract was washed successively with a 10% aqueous citric acid solution, water, a saturated aqueous sodium bicarbonate solution, water and brine and dried over anhydrous magnesium sulfate. The solvent was then evaporated in vacuo to give the desired product (8.21 g) as an oil.

¹H-NMR (CDCl₃) δ 1.20-1.28 (3H, m), 1.33-1.61 (9H, m), 3.09-3.20 (1H, m), 3.31-3.38 (1H, m), 3.84-4.21 (5H, m), 4.57-4.71 (2H, m), 4.96-5.01 (1H, m), 5.29-5.31 (1H, m), 6.84-6.94 (2H, m), 7.06-7.17 (3H, m), 7.26-7.31 (2H, m)

In the same manner as in Reference Example 92, the following compounds (Reference Examples 93 to 95) were obtained.

### Reference Example 93

### EthylN-(tert-butoxycarbonyl)-D-tyrosyl-N-benzylglycinate

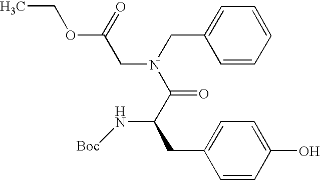

¹H-NMR (CDCl₃) δ 1.11-1.52 (12H, m), 3.66-4.26 (5H, m), 4.36-4.78 (3H, m), 4.83-5.13 (1H, m), 5.22-5.37 (1H, m), 5.65 (1H, br s), 6.61-7.49 (10H, m)

### Reference Example 94

### EthylN-(tert-butoxycarbonyl)-DL-tyrosyl-N-benzylglycinate

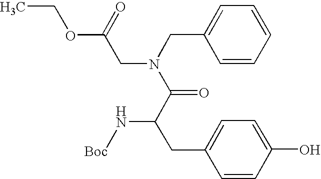

¹H-NMR (CDCl₃) δ 1.11-1.52 (12H, m), 3.66-4.26 (5H, m), 4.36-4.78 (3H, m), 4.83-5.13 (1H, m), 5.22-5.37 (1H, m), 5.65 (1H, br s), (10H, m)

### Reference Example 95

### EthylN-(tert-butoxycarbonyl)-2-methoxyphenylalanyl-N-benzylglycinate

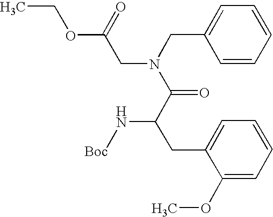

¹H-NMR (CDCl₃) δ 1.19-1.72 (12H, m), 2.50-3.31 (2H, m), 3.64-3.90 (3H, m), 4.00-4.27 (3H, m), 4.48-5.46 (3H, m), 6.75-6.92 (2H, m), 7.01-7.42 (7H, m)

### Reference Example 96

### (3R)-1-Benzyl-3-(2-thienylmethyl)piperazine-2,5-dione

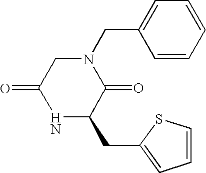

To a solution of ethyl N-(tert-butoxycarbonyl)-3-(2-thienyl)-D-alanyl-N-benzylglycinate (8.20 g) in dichloromethane (7 ml), was added TFA (70 ml) and stirred at room temperature for 30 min. The reaction mixture was concentrated in vacuo, and the residue was diluted with toluene and then further concentrated in vacuo to remove TFA. The residue was dissolved in dichloromethane (100 ml), triethylamine (20 ml) was added thereto, and the mixture was stirred at room temperature for 2.5 hr. The reaction mixture was concentrated in vacuo, and the residue was dissolved in ethyl acetate-THF (4:1, 250 ml), washed successively with a 10% aqueous citric acid solution, water, a saturated aqueous sodium bicarbonate solution, water and brine and dried over anhydrous magnesium sulfate. The solvent was, then evaporated in vacuo, and the crystals were collected by filtration to give the desired product (3.80 g). A portion thereof was recrystallized from ethyl acetate-hexane and taken as a sample for analysis.

¹H-NMR (CDCl₃) δ 3.27 (1H, d), 3.34 (1H, dd), 3.47 (1H, dd), 3.63 (1H, d), 4.35 (1H, s),4.51 (2H, s), 6.70 (1H, s), 6.85 (1H, d), 6.90 (1H, dd), 7.13-7.19 (3H, m), 7.29-7.31 (3H, m)

MS (ESI+, m/e) 301 (M+1)

In the same manner as in Reference Example 96, the following compounds (Reference Examples 97 to 99) were obtained.

### Reference Example 97

### (3R)-1-Benzyl-3-(4-hydroxybenzyl)piperazine-2,5-dione

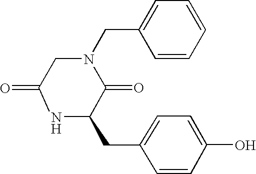

¹H-NMR (DMSO-d₆) δ 2.70-2.82 (1H, m), 2.99-3.11 (1H, m), 3.32-3.43 (2H, m), 4.14-4.26 (2H, m), 4.55 (1H, d), 6.52 (2H, d), 6.83 (2H, d), 7.11 (2H, m), 7.23-7.39 (3H, m), 8.23-8.31 (1H, m), 9.26 (1H, s)

### Reference Example 98

### 1-Benzyl-3-(4-hydroxybenzyl)piperazine-2,5-dione

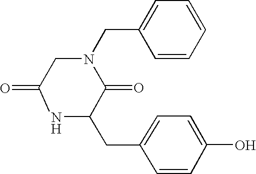

¹H-NMR (DMSO-d₆) δ 2.70-2.82 (1H, m), 2.99-3.11 (1H, m), 3.32-3.43 (2H, m), 4.14-4.26 (2H, m), 4.55 (1H, d), 6.52 (2H, d), 6.83 (2H, d), 7.11 (2H, m), 7.23-7.39 (3H, m), 8.23-8.31 (1H, m), 9.26 (1H, s)

### Reference Example 99

### 1-Benzyl-3-(2-methoxybenzyl)piperazine-2,5-dione

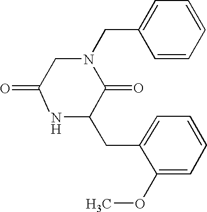

¹H-NMR (DMSO-d₆) δ 2.90-3.00 (1H, m), 3.04-3.19 (2H, m), 3.47 (1H, d), 3.74 (3H, s), 4.08-4.15 (1H, m), 4.43 (2H, s), 6.64-6.76 (4H, m), 6.96 (2H, dd), 8.09 (1H, br s)

### Reference Example 100

### 1-Benzyl-3-[4-(3-bromopropoxy)benzyl]piperazine-2,5-dione

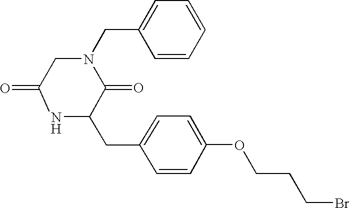

To a suspension of 1-benzyl-3-(4-hydroxybenzyl)piperazine-2,5-dione (2.0 g), potassium carbonate (1.34 g) and acetonitrile (10 ml), was added dibromopropane (2.6 g). After the mixture was heated under reflux for 12 hr, it was filtered, and the filtrate was concentrated in vacuo. The residue was subjected to silica gel column chromatography, and the fraction eluted with ethyl acetate-methanol (4:1) was concentrated in vacuo to give the desired product (1.5 g) as an amorphous solid.

¹H-NMR (CDCl₃) δ 2.23-2.37 (2H, m), 2.92-3.21 (3H, m), 3.49-3.67 (3H, m), 4.04 (2H, t), 4.26-4.34 (1H, m), 4.37-4.58 (2H, m), 6.18 (1H, br s), 6.63-6.78 (2H, m), 6.94-7.09 (2H, m), 7.15-7.42 (5H, m)

### Reference Example 101

### 1-Benzyl-3-[4-(3-methoxypropoxy)benzyl]piperazine-2,5-dione

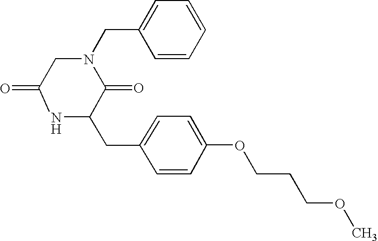

To a solution of 1-benzyl-3-[4-(3-bromopropoxy)benzyl]piperazine-2,5-dione (1.5 g) in methanol (10 ml), was added a 28% solution of sodium methoxide in methanol (1 ml). After stirring at 60° C. for 2 hr, the mixture was poured into ice-water. The solvent of the mixture was evaporated in vacuo, and the remaining aqueous solution was acidified with 3 N hydrochloric acid. The suspension was filtered, the crystals were subjected to silica gel column chromatography, and the fraction eluted with ethyl acetate-methanol (4:1) was concentrated in vacuo to give the desired product (1.0 g) as an amorphous solid.

¹H-NMR (CDCl₃) δ 1.90-2.14 (2H, m), 2.93-3.17 (3H, m), 3.37 (3H, s), 3.47-3.61 (2H, m), 3.92-4.05 (2H, m), 4.23-4.32 (1H, m), 4.41-4.57 (3H, m), 6.17 (1H, br s), 6.69-6.79 (2H, m), 6.98-7.08 (2H, m), 7.15-7.23 (2H, m), 7.28-7.37 (3H, m)

### Reference Example 102

### (3R)-1-Benzyl-3-(2-thienylmethyl)piperazine

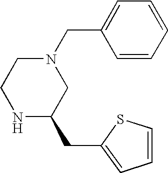

A mixture of (3R)-1-benzyl-3-(2-thienylmethyl)piperazine-2,5-dione (3.50 g) and THF (100 ml) was ice-cooled, and lithium aluminum hydride (1.77 g) was added portionwise thereto. After stirring at room temperature for 30 min and at 60° C. for 15 hr, the mixture was cooled to −78° C., and ethanol-ethyl acetate (1:1, 12 ml) and a 1 N aqueous sodium hydroxide solution (24 ml) were successively added dropwise thereto. After the completion of the dropwise addition, the reaction mixture was stirred at room temperature for 40 min. The insolubles were filtered off and washed with ethyl acetate. The filtrate was washed with brine and dried over anhydrous magnesium sulfate, and then the solvent was evaporated in vacuo. The residue was subjected to basic silica gel column chromatography, and the fraction eluted with ethyl acetate-hexane (1:1) was concentrated in vacuo to give the desired product (2.95 g) as an oil.

¹H-NMR (CDCl₃) δ 1.70 (1H, br s), 1.87 (1H, dd), 2.10 (1H, dt), 2.71-3.00 (7H, m), 3.50 (2H, dd), 6.83 (1H, d), 6.92 (1H, dd), 7.14 (1H, dd), 7.23-7.32 (5H, m)

In the same manner as in Reference Example 102, the following compounds (Reference Examples 103 to 106) were obtained.

### Reference Example 103

### 4-{[(2R)-4-Benzylpiperazin-2-yl]methyl}phenol

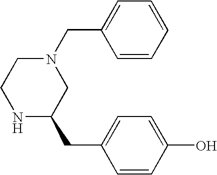

¹H-NMR (DMSO-d₆) δ 1.52-2.88 (8H, m), 3.08-3.73 (4H, m), 6.64 (2H, d), 6.94 (2H, d), 7.16-7.35 (5H, m), 9.17 (1H, br s)

MS (ESI+, m/e) 283 (M+1)

### Reference Example 104

### 4-[(4-Benzylpiperazin-2-yl)methyl]phenol

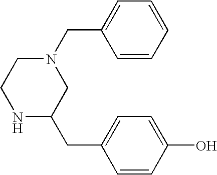

¹H-NMR (DMSO-d₆) δ 1.52-2.88 (8H, m), 3.08-3.73 (4H, m), 6.64 (2H, d), 6.94 (2H, d), 7.16-7.35 (5H, m), 9.17 (1H, br s)

MS (ESI+, m/e) 283 (M+1)

### Reference Example 105

### 1-Benzyl-3-[4-(3-methoxypropoxy)benzyl]piperazine

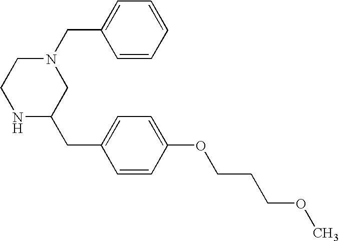

¹H-NMR (CDCl₃) δ 1.65-2.14 (4H, m), 2.39-2.52 (2H, m), 2.59-2.99 (5H, m), 3.35 (3H, s), 3.43-3.59 (4H, m), 3.66-3.72 (1H, m), 3.96-4.06 (2H, m), 6.83 (2H, d), 7.09 (2H, d), 7.21-7.38 (5H, m)

MS (ESI+, m/e) 355 (M+1)

### Reference Example 106

### 1-Benzyl-3-(2-methoxybenzyl)piperazine

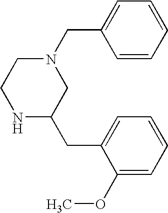

¹H-NMR (CDCl₃) δ 2.51-3.10 (9H, m), 3.40-3.61 (2H, m), 3.66-3.74 (1H, m), 3.80 (3H, s), 6.80-6.93 (4H, m), 7.09-7.36 (5H, m)

MS (ESI+, m/e) 297 (M+1)

### Reference Example 107

### BenzylN-benzyl-N-{(35)-3-[(tert-butoxycarbonyl)amino]-4-phenylbutanoyl}glycinate

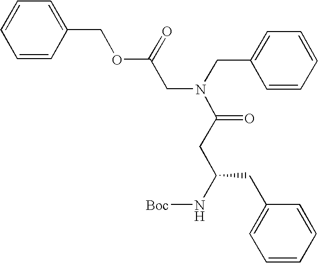

A solution of (3S)-3-[(tert-butoxycarbonyl)amino]-4-phenylbutyric acid (4.02 g), benzyl(benzylamino)acetate (3.67 g), WSC.HCl (3.31 g), HOBt (2.14 g) and DMF (70 ml) was stirred at room temperature for 15 hr. Then, the mixture was poured into water and extracted with ethyl acetate. The extract was washed successively with a 10% aqueous citric acid solution, water, a saturated aqueous sodium bicarbonate solution, water and brine and dried over anhydrous magnesium sulfate. Then, the solvent was evaporated in vacuo to give the desired product (7.41 g) as an oil.

MS (ESI+, m/e) 417 (M+1−Boc)

### Reference Example 108

### {[(3S)-3-Amino-4-phenylbutanoyl](benzyl)amino}acetic acid

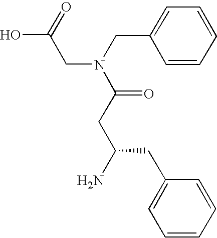

To a solution of benzyl N-benzyl-N-{(3S)-3-[(tert-butoxycarbonyl)amino]-4-phenylbutanoyl}glycinate (7.40 g) in dichloromethane (6 ml), was added TFA (60 ml) and stirred at room temperature for 30 min. The reaction mixture was concentrated in vacuo, diluted with a saturated aqueous sodium bicarbonate solution (250 ml), and then extracted with ethyl acetate. The extract was washed successively with water and brine and dried over anhydrous magnesium sulfate, and then the solvent was evaporated in vacuo. The residue was subjected to basic silica gel column chromatography, and the fraction eluted with ethyl acetate-hexane (1:2 to 1:1) was concentrated in vacuo to give an oil (4.45 g). The resulting oil was dissolved in methanol (90 ml), 20% palladium on carbon hydroxide (containing 50% water, 2.2 g) was added thereto, and the mixture was subjected to catalytic hydrogenation, at room temperature and atmospheric pressure for 3 hr. The catalyst was filtered off, the filtrate was concentrated in vacuo, and the crystals were collected by filtration to give the desired product (2.11 g).

MS (ESI+, m/e) 327 (M+1)

### Reference Example 109

### (7S)-4,7-Dibenzyl-1,4-diazepan-2,5-dione

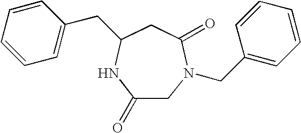

WSC.HCl (5.99 g) and HOBt (3.38 g) were dissolved in dichloromethane-DMF (4:1, 200 ml), and the mixture was stirred at room temperature for 30 min. {[(3S)-3-Amino-4-phenylbutanoyl](benzyl)amino)acetic acid (2.04 g) was added portionwise thereto while vigorously agitating the mixture over 20 min. After stirring at room temperature for 3 days, the reaction mixture was concentrated in vacuo, and the remaining solution poured into water and extracted with ethyl acetate. The extract was washed successively with a 10% aqueous citric acid solution, water, a saturated aqueous sodium bicarbonate solution, water and brine and dried over anhydrous magnesium sulfate. Then, the solvent was evaporated in vacuo, and the crystals were collected by filtration to give the desired product (1.68 g). A portion thereof was recrystallized from ethyl acetate-hexane and taken as a sample for analysis.

¹H-NMR (CDCl₃) δ 2.67 (1H, dd), 2.88-3.02 (3H, m), 3.86-3.91 (1H, m), 3.95 (1H, d), 4.07 (1H, d), 4.55 (1H, d), 4.76 (1H, d), 5.65 (1H, s), 7.16-7.38 (10H, m)

MS (ESI+, m/e) 309 (M+1)

### Reference Example 110

### (7S)-4,7-Dibenzyl-1,4-diazepan

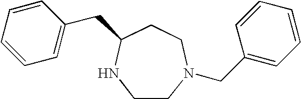

A mixture of (7S)-4,7-dibenzyl-1,4-diazepan-2,5-dione (1.54 g) and THF (45 ml) was ice-cooled, and lithium aluminum hydride (758 mg) was added portionwise thereto. After stirring at room temperature for 30 min and at 60° C. for 16 hr, the mixture was cooled to −78° C., and ethanol-ethyl acetate (1:1, 5 ml) and a 1 N aqueous sodium hydroxide solution (10 ml) were successively added dropwise thereto. After the completion of the dropwise addition, the reaction mixture was stirred at room temperature for 40 min. The insolubles were filtered off and washed with ethyl acetate. The filtrate was washed with brine and dried over anhydrous magnesium sulfate. Then, the solvent was evaporated in vacuo, the residue was subjected to basic silica gel column chromatography, and the fraction eluted with ethyl acetate-hexane (1:1) was concentrated in vacuo to give the desired product (1.19 g) as an oil.

¹H-NMR (CDCl₃) δ 1.54-1.66 (2H, m), 1.80-1.90 (1H, m), 2.56-2.78 (7H, m), 2.91-2.99 (1H, m), 3.07-3.17 (1H, m), 3.63 (2H, dd), 7.18-7.38 (10H, m)

### Reference Example 111

### tert-Butyl4-benzyl-3-{[(methylsulfonyl)oxy]methyl}piperazine-1-carboxylate

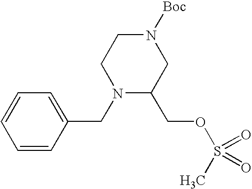

tert-Butyl4-benzyl-3-(hydroxymethyl)piperazine-1-carboxylate (3.06 g) and triethylamine (1.52 g) were dissolved in dichloromethane (20 ml), and methanesulfonyl chloride (1.43 g) was added dropwise thereto at 0° C. for 5 min. After stirring at room temperature for 15 hr, the mixture was washed with water and brine and dried over anhydrous magnesium sulfate. The solvent was evaporated in vacuo to give the desired product (3.07 g) as an oil.

MS (ESI+, m/e) 385 (M+1)

### Reference Example 112

### tert-Butyl3-{[(3-amino-2,2-dimethyl-3-oxopropyl)amino]methyl}-4-benzylpiperazine-1-carboxylate

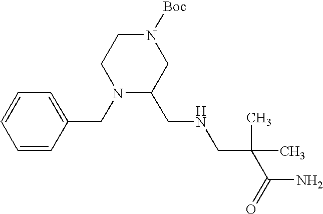

tert-Butyl4-benzyl-3-{[(methylsulfonyl)oxy]methyl}piperazine-1-carboxylate (960 mg), potassium carbonate (415 mg) and 3-amino-2,2-dimethylpropanamide (348 mg) were suspended in acetonitrile (10 ml) and stirred at 60° C. for 15 hr. The reaction mixture was concentrated in vacuo, diluted with water and extracted with ethyl acetate. The extract was washed successively with water and brine and dried over anhydrous magnesium sulfate, and then the solvent was evaporated in vacuo. The residue was subjected to silica gel column chromatography, the target fraction was concentrated in vacuo, and the crystals were collected by filtration to give the desired product (450 mg).

MS (ESI+, m/e) 405 (M+1)

### Reference Example 113

### tert-Butyl3-{[(3-amino-2,2-dimethyl-3-oxopropyl)(tert-butoxycarbonyl)amino]methyl}-4-benzylpiperazine-1-carboxylate

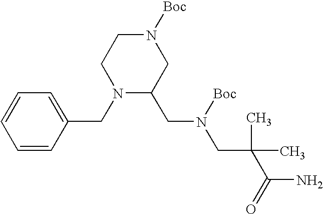

tert-Butyl3-{[(3-amino-2,2-dimethyl-3-oxopropyl)amino]methyl}-4-benzylpiperazine-1-carboxylate (809 mg) was dissolved in 1,2-dimethoxyethane (10 ml). A 1 N aqueous sodium hydroxide solution (4 ml) and di-tert-butyl dicarbonate (1.1 g) were added thereto and stirred at room temperature for 15 hr. The reaction mixture was concentrated in vacuo, diluted with water extracted with ethyl acetate, and dried over anhydrous magnesium sulfate. The solvent was then evaporated in vacuo, the residue was subjected to silica gel column chromatography, and the target fraction was concentrated in vacuo to give the desired product (600 mg) as an amorphous solid.

MS (ESI+, m/e) 505 (M+1)

### Reference Example 114

### tert-Butyl3-{[(3-amino-2,2-dimethyl-3-oxopropyl)(tert-butoxycarbonyl)amino]methyl}piperazine-1-carboxylate

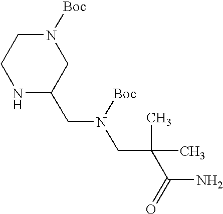

tert-Butyl3-{[(3-amino-2,2-dimethyl-3-oxopropyl)(tert-butoxycarbonyl)amino]methyl}-4-benzylpiperazine-1-carboxylate (600 mg) was dissolved in ethanol (10 ml), 20% palladium on carbon hydroxide (containing 50% water, 120 mg) was added thereto, and the mixture was subjected to catalytic hydrogenation at room temperature and atmospheric pressure for 15 hr. The catalyst was filtered off, and the filtrate was concentrated in vacuo. The residue was subjected to silica gel column chromatography, the target fraction was concentrated in vacuo, and the crystals were collected by filtration to give the desired product (435 mg).

MS (ESI+, m/e) 505 (M+1)

### Reference Example 115

### tert-Butyl3-(phenoxymethyl)piperazine-1-carboxylate

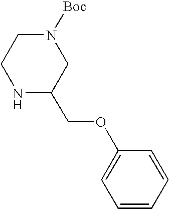

To a solution of tert-butyl4-benzyl-3-{[(methylsulfonyl)oxy]methyl}piperazine-1-carboxylate (1.15 g) in acetonitrile (10 ml) were added phenol (423 mg) and potassium carbonate (622 mg), and the mixture was stirred at room temperature for 15 hr. The insolubles were filtered off, and the filtrate was concentrated in vacuo. The residue was subjected to silica gel column chromatography, and the target fraction was concentrated in vacuo to give an amorphous solid (1.05 g). A portion thereof (560 mg) was dissolved in ethanol (10 ml), 10% palladium on carbon (containing 50% water, 110 mg) was added thereto, and the mixture was subjected to catalytic hydrogenation at room temperature and atmospheric pressure for 15 hr. The catalyst was filtered off, and the filtrate was concentrated in vacuo. The residue was subjected to silica gel column chromatography, the target fraction was concentrated in vacuo, and the crystals were collected by filtration to give the desired product (305 mg).

MS (ESI+, m/e) 293 (M+1)

In the same manner as in Reference Example 115, the following compounds (Reference Examples 116 to 120) were’ obtained.

### Reference Example 116

### tert-Butyl3-{[4-(methoxycarbonyl)phenoxy]methyl}piperazine-1-carboxylate

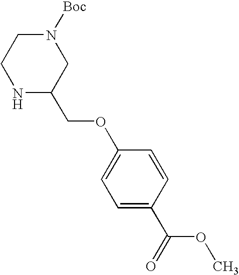

MS (ESI+, m/e) 351 (M+1)

### Reference Example 117

### tert-Butyl3-{[3-(methoxycarbonyl)phenoxy]methyl}piperazine-1-carboxylate

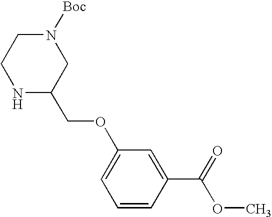

MS (ESI+, m/e) 351 (M+1)

### Reference Example 118

### tert-Butyl3-{[4-(2-methoxy-2-oxoethyl)phenoxy]methyl}piperazine-1-carboxylate

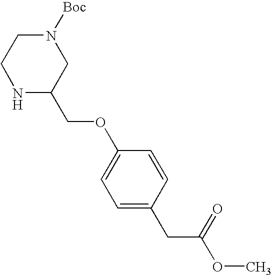

MS (ESI+, m/e) 364 (M+1)

### Reference Example 119

### tert-Butyl3-{[3-(3-methoxy-3-oxopropyl)phenoxy]methyl}piperazine-1-carboxylate

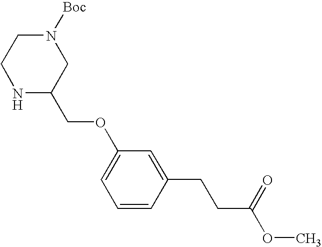

MS (ESI+, m/e) 379 (M+1)

### Reference Example 120

### tert-Butyl3-{[(1,3-benzodioxol-5-yl)oxy]methyl}piperazine-1-carboxylate

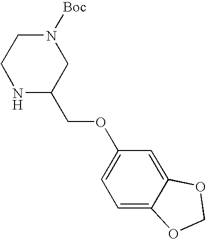

MS (ESI+, m/e) 337 (4+1)

### Reference Example 121

### tert-Butyl4-benzyl-3-[(1H-imidazol-1-yl)methyl]piperazine-1-carboxylate

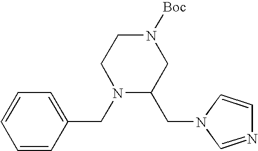

A solution of tert-butyl4-benzyl-3-{[(methylsulfonyl)oxy]methyl}piperazine-1-carboxylate (1.15 g) and imidazol-1-yl sodium (540 mg) in DMF (10 ml) was stirred at 60° C. for 15 hr. The reaction mixture was poured into water and extracted with ethyl acetate. The extract was washed successively with water and brine and dried over anhydrous magnesium sulfate, and then the solvent was evaporated in vacuo. The residue was subjected to silica gel column chromatography, and the target fraction was concentrated in vacuo to give the desired product (590 mg) as an oil.

MS (ESI+, m/e) 357 (M+1)

### Reference Example 122

### tert-Butyl3-[(1H-imidazol-1-yl)methyl]piperazine-1-carboxylate

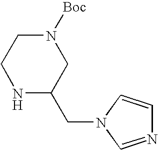

tert-Butyl4-benzyl-3-[(1H-imidazol-1-yl)methyl]piperazine-1-carboxylate (580 mg) was dissolved in ethanol (5 ml), 10% palladium on carbon (containing 50% water, 100 mg) was added thereto, and the mixture was subjected to catalytic hydrogenation at room temperature and atmospheric pressure for 15 hr. The catalyst was filtered off, and the filtrate was concentrated in vacuo. The residue was subjected to silica gel column chromatography, and the target fraction was concentrated in vacuo to give the desired product (390 mg) as an oil.

MS (ESI+, m/e) 357 (M+1)

### Reference Example 123

### tert-Butyl(2R)-4-benzyl-2-(4-hydroxybenzyl)piperazine-1-carboxylate

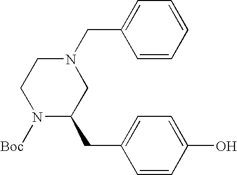

4-{[(2R)-4-Benzylpiperazin-2-yl]methyl}phenol (2.82 g) and N,N-diisopropylethylamine (2.59 g) were dissolved in THF (30 ml), di-tert-butyl dicarbonate (2.18 g) was added thereto at 0° C., and the mixture was stirred at room temperature for 15 hr. The solvent was evaporated in vacuo, and ethyl acetate (50 ml) was added to the residue and dissolved. The reaction mixture was washed successively with water and brine and dried over anhydrous magnesium sulfate, and then the solvent was evaporated in vacuo. The residue was subjected to silica gel column chromatography, the target fraction was concentrated in vacuo, and the crystals were collected by filtration to give the desired product (3.1 g).

MS (ESI+, m/e) 383 (M+1)

### Reference Example 124

### tert-Butyl(2R)-4-benzyl-2-(4-{[(trifluoromethyl)sulfonyl]oxy}benzyl)piperazine-1-carboxylate

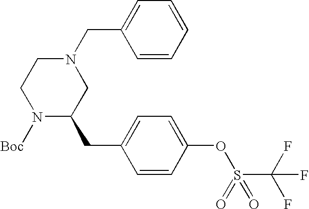

tert-Butyl(2R)-4-benzyl-2-(4-hydroxybenzyl)piperazine-1-carboxylate (3.06 g), potassium carbonate (2.2 g) and 4-nitrophenyl trifluoromethanesulfonate (2.39 g) were suspended in DMF (50 ml), and the suspension was stirred at room temperature for 15 hr. Then, the reaction mixture was poured into water and extracted with ethyl acetate. The extract was washed successively with water and brine and dried over anhydrous magnesium sulfate, and then the solvent was evaporated in vacuo. The residue was subjected to silica gel column chromatography, and the target fraction was concentrated in vacuo to give the desired product (3.75 g) as an oil.

MS (ESI+, m/e) 515 (M+1)

### Reference Example 125

### tert-Butyl(2R)-4-benzyl-2-[4-(ethoxycarbonyl)benzyl]piperazine-1-carboxylate

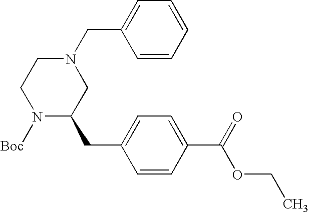

tert-Butyl(2R)-4-benzyl-2-(4-{[(trifluoromethyl)sulfonyl]oxy}benzyl)piperazine-1-carboxylate (3.0 g), triethylamine (2.12 ml), palladium acetate (67 mg) and dppf (166 mg) were suspended in ethanol (30 ml), and the suspension was stirred at 70° C. for 18 hr under carbon monoxide atmosphere. The reaction mixture was cooled to room temperature and diluted with ethyl acetate (50 ml) and water (30 ml), and then the insolubles were filtered off using Celite. The organic layer was separated, washed with brine and dried over magnesium sulfate, and then the solvent was evaporated in vacuo. The residue was subjected to silica gel column chromatography, the fraction eluted with ethyl acetate-hexane (1:5 to 1:3) was concentrated in vacuo, and the crystals were collected by filtration to give the desired product (1.95 g).

MS (ESI+, m/e) 439 (M+1)

### Reference Example 126

### Ethyl4-{[(2R)-4-benzylpiperazin-2-yl]methyl}benzoate

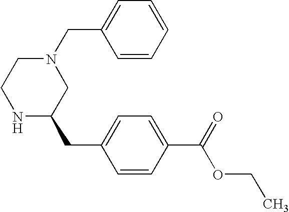

tert-Butyl(2R)-4-benzyl-2-[4-(ethoxycarbonyl)benzyl]piperazine-1-carboxylate (1.9 g) was dissolved in dichloromethane (1 ml), TEA (5 ml) was added thereto and stirred at room temperature for 1 hr. Then, the reaction mixture was concentrated in vacuo, and neutralized by adding a 6% aqueous sodium bicarbonate solution to the residue. The liberated oil was extracted with chloroform. The extract was washed with brine and dried over anhydrous magnesium sulfate, and then the solvent was evaporated in vacuo. The residue was subjected to silica gel column chromatography, the target fraction was concentrated in vacuo, and the crystals were collected by filtration to give the desired product (1.3 g).

MS (ESI+, m/e) 339 (M+1)

### Reference Example 127

### Ethyl{4-[3-{[(2R)-2,4-dibenzylpiperazin-1-yl]carbonyl}-1-(2,3-dimethoxyphenyl)-5-methyl-1H-pyrrol-2-yl]phenoxy}acetate

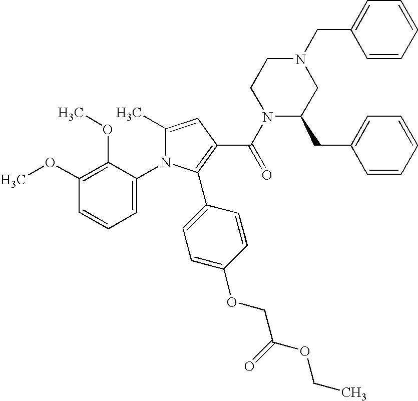

A solution of 1-(2,3-dimethoxyphenyl)-2-[4-(2-ethoxy-2-oxoethoxy)phenyl]-5-methyl-1H-pyrrole-3-carboxylic acid (944 mg), (3R)-1,3-dibenzylpiperazine (572 mg), WSC.HCl (494 mg), HOBt (348 mg) and DMF (15 ml) was stirred at room temperature for 18 hr. Then, the reaction mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to basic silica gel column chromatography, and the fraction eluted with ethyl acetate-hexane (1:1) was concentrated in vacuo to give the desired product (1.11 g) as an oil.

MS (ESI+, m/e) 688 (M+1)

In the same manner as in Reference Example 127, the following compounds (Reference Examples 128 to 138) were obtained.

### Reference Example 128

### (2R)-2,4-Dibenzyl-1-{[1-(3-bromophenyl)-5-methyl-2-phenyl-1H-pyrrol-3-yl]carbonyl}piperazine

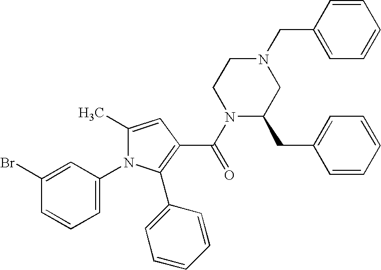

MS (ESI+, m/e) 604 (M+1)

### Reference Example 129

### (2R)-2,4-Dibenzyl-1-({1-[2-(benzyloxy)phenyl]-5-methyl-2-phenyl-1H-pyrrol-3-yl}carbonyl)piperazine

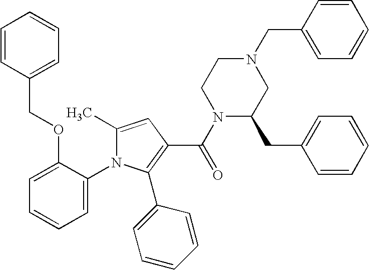

MS (ESI+, m/e) 632 (M+1)

### Reference Example 130

### tert-Butyl(2R)-(2-{3-[(2,4-dibenzylpiperazin-1-yl)carbonyl]-5-methyl-2-phenyl-1H-pyrrol-1-yl}benzyl)carbamate

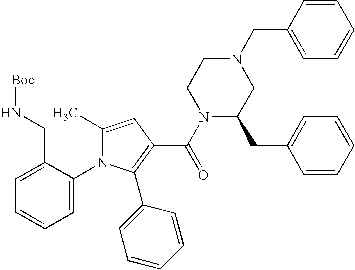

MS (ESI+, m/e) 655 (M+1)

### Reference Example 131

### (2R)-2,4-Dibenzyl-1-[(2-phenyl-1H-pyrrol-3-ylcarbonyl]piperazine

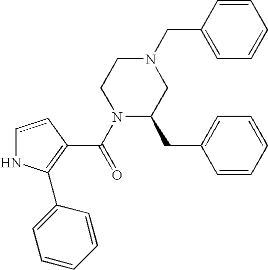

MS (ESI+, m/e) 436 (M+1)

### Reference Example 132

### 4-Benzyl-1-{[1-(3-bromophenyl)-5-methyl-2-phenyl-1H-pyrrol-3-yl]carbonyl}-2-(2-methoxybenzyl)piperazine

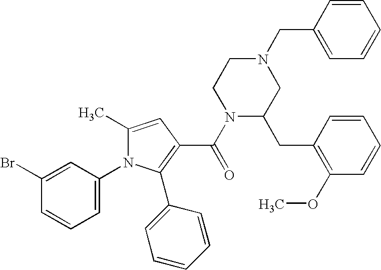

MS (ESI+, m/e) 634 (M+1)

### Reference Example 133

### Methyl3-(3-{[(2R)-2,4-dibenzylpiperazin-1-yl]carbonyl}-5-methyl-2-phenyl-1H-pyrrol-1-yl)-4,5-dimethoxybenzoate

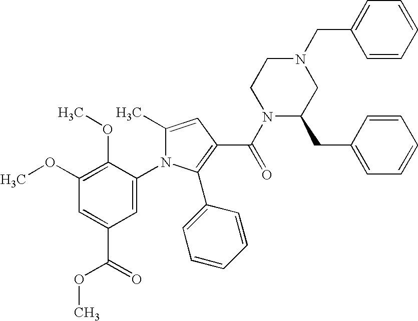

MS (ESI+, m/e) 644 (M+1)

### Reference Example 134

### Ethyl3-[3-(3-{[(2R)-2,4-dibenzylpiperazin-1-yl]carbonyl}-5-methyl-2-phenyl-1H-pyrrol-1-yl)-4,5-dimethoxyphenyl]propanoate

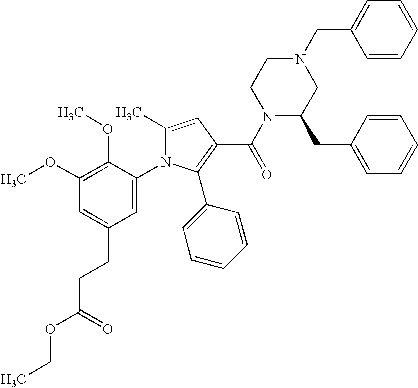

MS (ESI+, m/e) 686 (M+1)

### Reference Example 135

### 4-((4-Benzyl-1-[(5-methyl-1,2-diphenyl-1H-pyrrol-3-yl)carbonyl]piperazin-2-yl}methyl)phenol

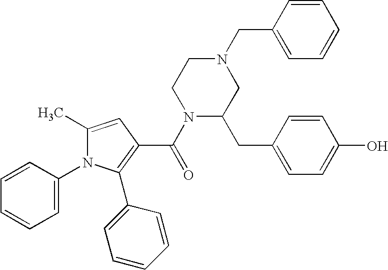

MS (ESI+, m/e) 542 (M+1)

### Reference Example 136

### Ethyl4-[((2R)-4-benzyl-1-{[1-(3-morpholinophenyl)-5-phenyl-1H-imidazol-4-yl]carbonyl}piperazin-2-yl)methyl]benzoate

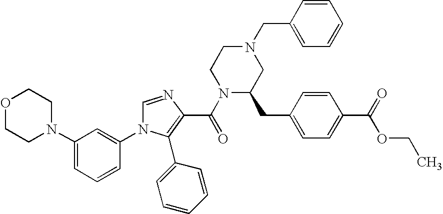

MS (ESI+, m/e) 670 (M+1)

### Reference Example 137

### {(2R)-4-Benzyl-1-[(5-methyl-1,2-diphenyl-1H-pyrrol-3-yl)carbonyl]piperazin-2-yl}methanol

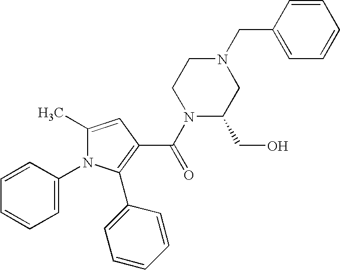

MS (ESI+, m/e) 466 (M+1)

### Reference Example 138

### {(2S)-4-Benzyl-1-[(5-methyl-1,2-diphenyl-1H-pyrrol-3-yl)carbonyl]piperazin-2-yl}methanol

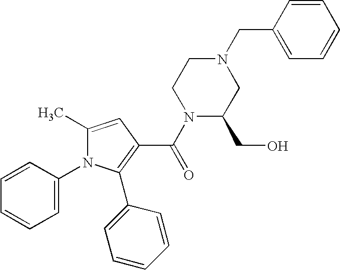

MS (ESI+, m/e) 466 (M+1)

### Reference Example 139

### tert-Butyl3-benzyl-4-{[1-(4-nitrophenyl)-2-phenyl-1H-pyrrol-3-yl]carbonyl}piperazine-1-carboxylate

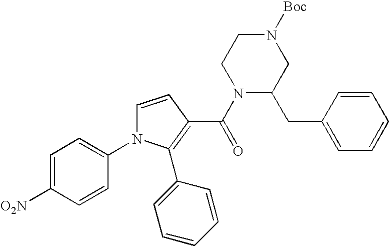

A solution of 1-(4-nitrophenyl)-2-phenyl-1H-pyrrole-3-carboxylic acid (190 mg), tert-butyl3-benzylpiperazine-1-carboxylate (170 mg), WSC.HCl (154 mg), HOBt (123 mg) and DMF (5 ml) was stirred at room temperature for 12 hr. The reaction mixture was poured into a saturated aqueous sodium bicarbonate solution and extracted with ethyl acetate. The extract was washed successively with water and brine and dried over anhydrous sodium sulfate, and then the solvent was evaporated in vacuo. The residue was subjected to silica gel column chromatography, and the fraction eluted with ethyl acetate-hexane (1:4 to 1:1) was concentrated in vacuo to give the desired product (230 mg) as an amorphous solid.

MS (ESI+, m/e) 567 (M+1)

In the same manner as in Reference Example 139, the following compounds (Reference Examples 140 to 154) were obtained.

### Reference Example 140

### tert-Butyl(3R)-3-benzyl-4-{[1-(5-hydroxy-2-nitrophenyl)-2-phenyl-1H-pyrrol-3-yl]carbonyl}piperazine-1-carboxylate

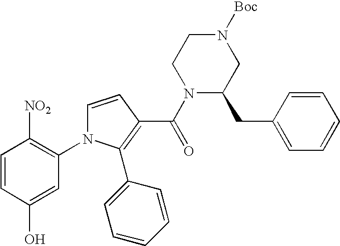

MS (ESI+, m/e) 583 (M+1)

### Reference Example 141

### tert-Butyl(3R)-3-benzyl-4-{[1-(3-bromophenyl)-5-methyl-2-phenyl-1H-pyrrol-3-yl]carbonyl}piperazine-1-carboxylate

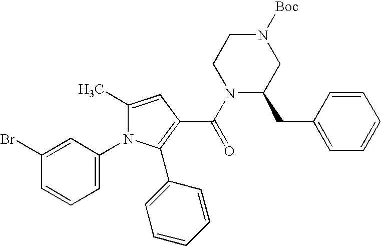

MS (ESI+, m/e) 615 (M+1)

### Reference Example 142

### tert-Butyl(3R)-3-benzyl-4-{[1-(3-hydroxyphenyl)-5-methyl-2-phenyl-1H-pyrrol-3-yl]carbonyl}piperazine-1-carboxylate

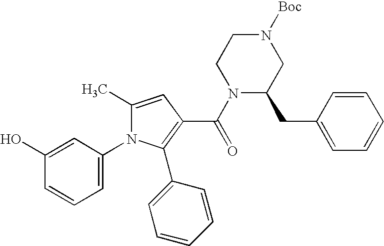

MS (ESI+, m/e) 552 (M+1)

### Reference Example 143

### tert-Butyl4-{[1-(3-bromophenyl)-5-methyl-2-phenyl-1H-pyrrol-3-yl]carbonyl}-3-(2-methoxybenzyl)piperazine-1-carboxylate

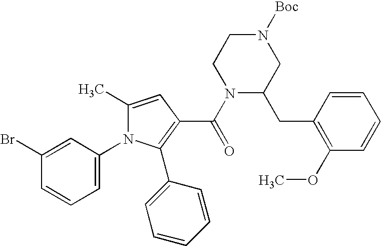

MS (ESI+, m/e) 644 (M+1)

### Reference Example 144

### tert-Butyl(3R)-3-benzyl-4-({2-[3-(benzyloxy)phenyl]-5-methyl-1-phenyl-1H-pyrrol-3-yl}carbonyl)piperazine-1-carboxylate

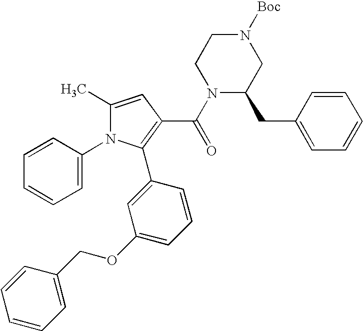

¹H-NMR (CDCl₃) δ 1.44 (9H, s), 2.10 (3H, s), 2.56-2.96 (4H, m), 3.51-4.13 (4H, m), 4.48-4.88 (3H, m), 5.79-6.17 (2H, m), 6.63-6.73 (3H, m), 6.91-7.34 (15H, m)

### Reference Example 145

### tert-Butyl3-benzyl-4-{[1-[2-(benzyloxy)phenyl]-5-(4-fluorophenyl)-1H-pyrazol-4-yl]carbonyl}piperazine-1-carboxylate

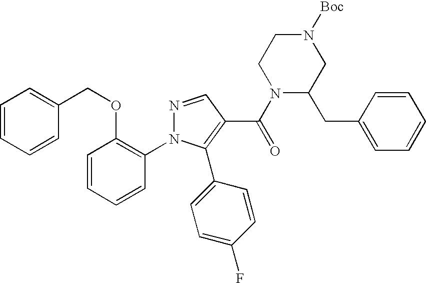

MS (ESI+, m/e) 647 (M+1)

### Reference Example 146

### tert-Butyl3-benzyl-4-({1-[3-(benzyloxy)phenyl]-5-phenyl-1H-1,2,3-triazol-4-yl}carbonyl)piperazine-1-carboxylate

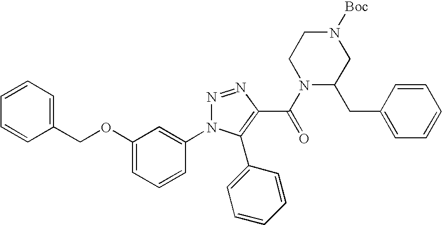

MS (ESI+, m/e) 630 (M+1)

### Reference Example 147

### tert-Butyl(3R)-3-benzyl-4-({1-[3-(benzyloxy)phenyl]-5-phenyl-1H-1,2,3-triazol-4-yl}carbonyl)piperazine-1-carboxylate

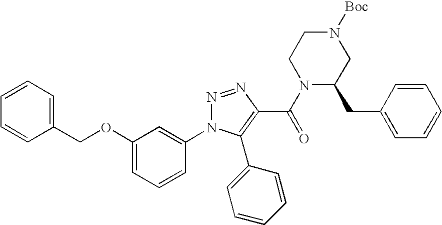

MS (ESI+, m/e) 630 (M+1)

### Reference Example 148

### tert-Butyl3-benzyl-4-({1-[4-(benzyloxy)phenyl]-5-phenyl-1H-1,2,3-triazol-4-yl}carbonyl)piperazine-1-carboxylate

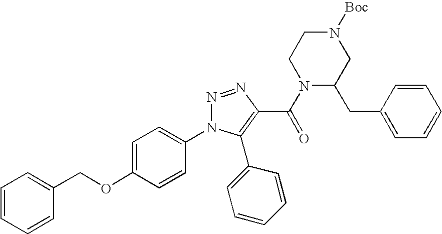

MS (ESI+, m/e) 630 (M+1)

### Reference Example 149

### tert-Butyl3-benzyl-4-({1-[2-(benzyloxy)phenyl]-5-phenyl-1H-1,2,3-triazol-4-yl}carbonyl)piperazine-1-carboxylate

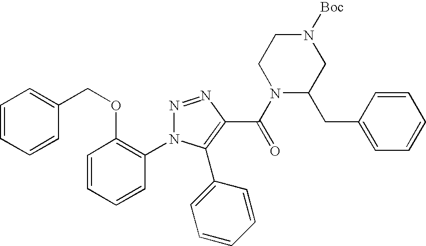

MS (ESI+, m/e) 630 (M+1)

### Reference Example 150

### tert-Butyl3-{[4-(methoxycarbonyl)phenoxy]methyl}-4-[(5-methyl-1,2-diphenyl-1H-pyrrol-3-yl)carbonyl]piperazine-1-carboxylate

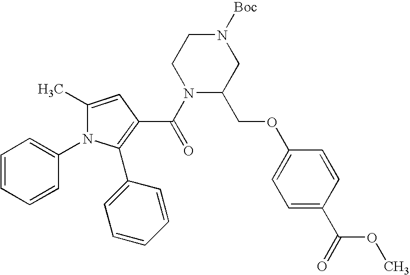

MS (ESI+, m/e) 610 (M+1)

### Reference Example 151

### tert-Butyl3-{[4-(2-methoxy-2-oxoethyl)phenoxy]methyl}-4-[(5-methyl-1,2-diphenyl-1H-pyrrol-3-yl)carbonyl)piperazine-1-carboxylate

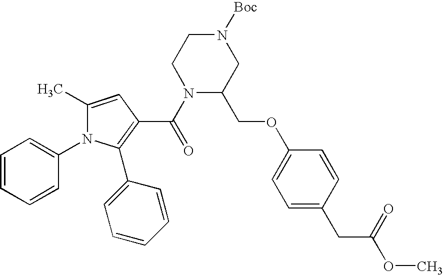

MS (ESI+, m/e) 624 (M+1)

### Reference Example 152

### tert-Butyl3-{[4-(3-methoxy-3-oxopropyl)phenoxy]methyl}-4-[(5-methyl-1,2-diphenyl-1H-pyrrol-3-yl)carbonyl]piperazine-1-carboxylate

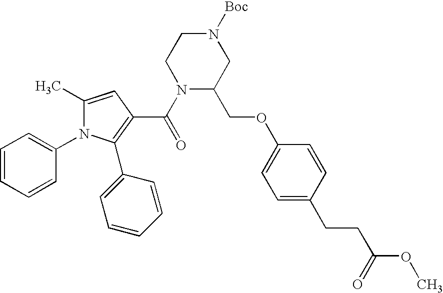

MS (ESI+, m/e) 638 (M+1)

### Reference Example 153

### tert-Butyl3-{[3-(methoxycarbonyl)phenoxy]methyl}-4-[(5-methyl-1,2-diphenyl-1H-pyrrol-3-yl)carbonyl]piperazine-1-carboxylate

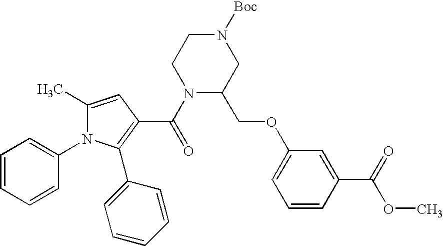

MS (ESI+, m/e) 610 (M+1)

### Reference Example 154

### 3-Ethyl1-tert-butyl4-[(5-methyl-1,2-diphenyl-1H-pyrrol-3-yl)carbonyl]piperazine-1,3-dicarboxylate

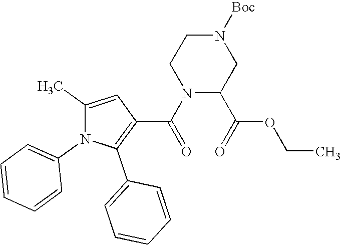

MS (ESI+, m/e) 518 (M+1)

### Reference Example 155

### 4-({4-Benzyl-1-[(5-methyl-1,2-diphenyl-1H-pyrrol-3-yl)carbonyl]piperazin-2-yl}methyl)phenyl trifluoromethanesulfonate

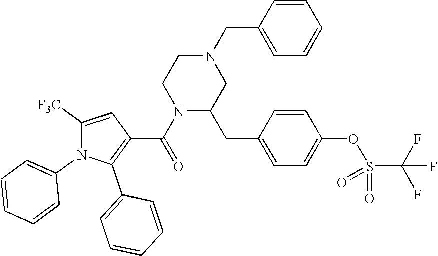

4-({4-Benzyl-1-[(5-methyl-1,2-diphenyl-1H-pyrrol-3-yl)carbonyl]piperazin-2-yl}methyl)phenol (1.63 g), potassium carbonate (829 mg) and 4-nitrophenyl trifluoromethanesulfonate (976 mg) were suspended in DMF (20 ml), and the suspension was stirred at room temperature for 15 hr. The mixture was poured into water and extracted with ethyl acetate. The extract was washed successively with water and brine and dried over anhydrous magnesium sulfate, and then the solvent was evaporated in vacuo. The residue was subjected to silica gel column chromatography, and the target fraction was concentrated in vacuo to give the desired product (1.7 g) as an oil.

MS (ESI+, m/e) 674 (M+1)

### Reference Example 156

### Ethyl4-({4-benzyl-1-[(5-methyl-1,2-diphenyl-1H-pyrrol-3-yl)carbonyl]piperazin-2-yl}methyl)benzoate

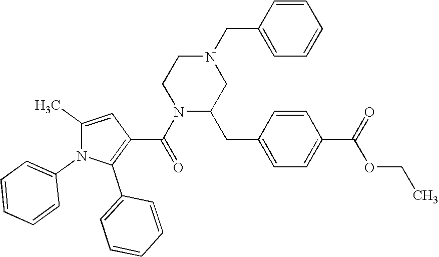

4-({4-Benzyl-1-[(5-methyl-1,2-diphenyl-1H-pyrrol-3-yl)carbonyl]piperazin-2-yl}methyl)phenyl trifluoromethanesulfonate (674 mg), triethylamine (354 mg), palladium acetate (11 mg) and dppf (28 mg) were suspended in ethanol (5 ml), and the suspension was stirred at 70° C. for 18 hr under carbon monoxide atmosphere. The reaction mixture was cooled to room temperature and diluted with ethyl acetate and water, and then the insolubles were filtered off using Celite. The organic layer was separated, washed with brine and dried over magnesium sulfate, and then the solvent was evaporated in vacuo. The residue was subjected to silica gel column chromatography, the fraction eluted with ethyl acetate-hexane (1:2 to 1:1) was concentrated in vacuo, and the crystals were collected by filtration to give the desired product (580 mg).

MS (ESI+, m/e) 598 (14+1)

### Reference Example 157

### tert-Butyl(3R)-4-{[1-(3-aminophenyl)-5-methyl-2-phenyl-1H-pyrrol-3-yl]carbonyl}-3-benzylpiperazine-1-carboxylate

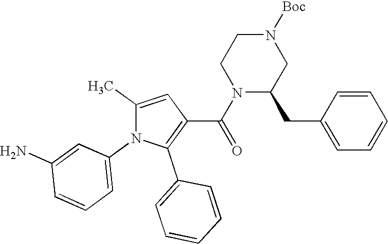

A solution of 5-methyl-1-(3-nitrophenyl)-2-phenyl-1H-pyrrole-3-carboxylic acid (3.55 g), tert-butyl(3R)-3-benzylpiperazine-1-carboxylate (3.04 g), WSC.HCl (2.53 g), HOBt (1.64 g) and DMF (55 ml) was stirred at room temperature for 15 hr. The reaction mixture was poured into water and extracted with ethyl acetate. The extract was washed successively with a 10% aqueous citric acid solution, water, a saturated aqueous sodium bicarbonate solution, water and brine and dried over anhydrous magnesium sulfate. Then, the solvent was evaporated in vacuo and the residue was subjected to silica gel column chromatography. The fraction eluted with ethyl acetate-hexane (1:2.5 to 2:1) was concentrated in vacuo to give an amorphous solid (5.05 g). The resulting amorphous was dissolved in methanol (130 ml), 10% palladium on carbon (containing 50% water, 2.3 g) was added thereto, and the mixture was subjected to catalytic hydrogenation at room temperature and atmospheric pressure for 3 hr. The catalyst was filtered off, and the filtrate was concentrated in vacuo. The residue was subjected to silica gel column chromatography, and the fraction eluted with ethyl acetate-hexane (1:1) was concentrated in vacuo to give the desired product (4.44 g) as an amorphous solid.

MS (ESI+, m/e) 551 (M+1)

### Reference Example 158

### tert-Butyl (3R)-3-benzyl-4-{[1-(5-methoxy-2-nitrophenyl)-2-phenyl-1H-pyrrol-3-yl]carbonyl}piperazine-1-carboxylate

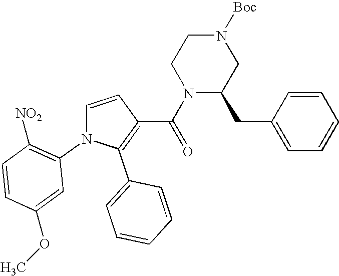

To a solution of tert-butyl (3R)-3-benzyl-4-{[1-(5-hydroxy-2-nitrophenyl)-2-phenyl-1H-pyrrol-3-yl]carbonyl}piperazine-1-carboxylate (1.1 g), potassium carbonate (785 mg) and 1,4-dioxane (10 ml), was added dimethyl sulfate (360 mg). After stirring at 80° C. for 12 hr, the mixture was poured into water, and the suspension was filtered. The crystals were dissolved in ethyl acetate and dried over anhydrous sodium sulfate, and the solvent was evaporated in vacuo. The residue was subjected to basic silica gel column chromatography, and the fraction eluted with ethyl acetate-hexane (1:1) was concentrated in vacuo to give the desired product (720 mg) as an amorphous solid.

MS (ESI+, m/e) 597 (M+1)

### Reference Example 159

### tert-Butyl 4-{[1-(4-aminophenyl)-2-methyl-1H-pyrrol-3-yl]carbonyl}-3-benzylpiperazine-1-carboxylate

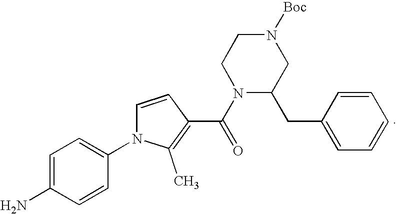

To a solution of tert-butyl 3-benzyl-4-{(2-methyl-1-(4-nitrophenyl)-1H-pyrrol-3-yl]carbonyl}piperazine-1-carboxylate (170 mg) in methanol (5 ml), was added 10% palladium on carbon (containing 50% water, 70 mg), and the mixture was subjected to catalytic hydrogenation at room temperature and atmospheric pressure for 12 hr. The catalyst was filtered off, and the filtrate was concentrated in vacuo. The residue was subjected to silica gel column chromatography, and the fraction eluted with ethyl acetate-hexane (1:1) was concentrated in vacuo to give the desired product (130 mg) as an amorphous solid.

MS (ESI+, m/e) 475 (M+1)

In the same manner as in Reference Example 159, the following compounds (Reference Examples 160 to 162) were obtained.

### Reference Example 160

### tert-Butyl (3R)-4-{[1-(2-amino-5-methoxyphenyl)-2-phenyl-1H-pyrrol-3-yl]carbonyl}-3-benzylpiperazine-1-carboxylate

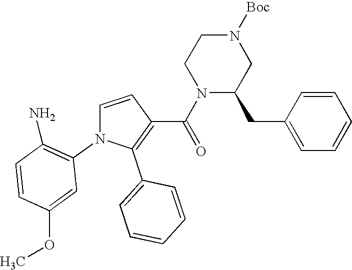

MS (ESI+, m/e) 567 (M+1)

### Reference Example 161

### tert-Butyl 4-{[1-(2-aminophenyl)-2-phenyl-1H-pyrrol-3-yl]carbonyl}-3-benzylpiperazine-1-carboxylate

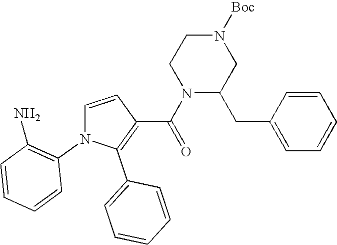

MS (ESI+, m/e) 537 (M+1)

### Reference Example 162

### tert-Butyl 4-{[1-(4-aminophenyl)-2-phenyl-1H-pyrrol-3-yl]carbonyl}-3-benzylpiperazine-1-carboxylate

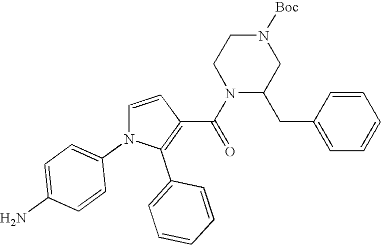

MS (ESI+, m/e) 537 (M+1)

### Reference Example 163

### tert-Butyl 3-benzyl-4-[(2-methyl-1-{4-[(5-phenylpentanoyl)amino]phenyl}-1H-pyrrol-3-yl)carbonyl]piperazine-1-carboxylate

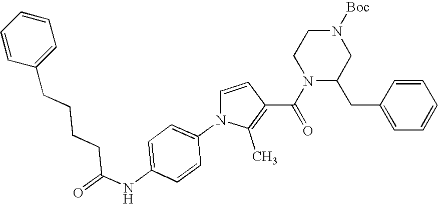

To a solution of 5-phenylpentanoic acid (41 mg) in toluene (2 ml) were added DMF (20 mg) and thionyl chloride (82 mg). After stirring at 80° C. for 1 hr, the solvent was evaporated in vacuo. The residue was dissolved In dichloromethane (2 ml), and the reaction mixture was added to a solution of ice-cooled tert-butyl 4-{[1-(4-aminophenyl)-2-methyl-1H-pyrrol-3-yl]carbonyl}-3-benzylpiperazine-1-carboxylate (100 mg), triethylamine (32 mg) and dichloromethane (3 ml). After stirring at room temperature for 1 hr, the mixture was poured into water and extracted with chloroform. The extract was dried over anhydrous sodium sulfate, and the solvent was evaporated in vacuo. The residue was subjected to silica gel column chromatography, and the fraction eluted with ethyl acetate-hexane (1:2) was concentrated in vacuo to give the desired product (102 mg) as an amorphous solid.

MS (ESI+, m/e) 635 (M+1)

In the same manner as in Reference Example 163, the following compounds (Reference Examples 164 to 165) were obtained.

### Reference Example 164

### tert-Butyl 3-benzyl-4-[(2-phenyl-1-{2-[(5-phenylpentanoyl)amino]phenyl}-1H-pyrrol-3-yl)carbonyl]piperazine-1-carboxylate

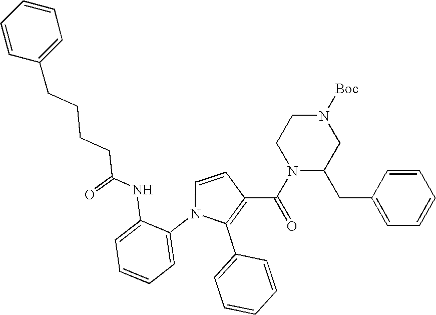

MS (ESI+, m/e) 697 (M+1)

### Reference Example 165

### tert-Butyl 3-benzyl-4-{(2-phenyl-1-{4-[(5-phenylpentanoyl)amino]phenyl}-1H-pyrrol-3-yl)carbonyl]piperazine-1-carboxylate

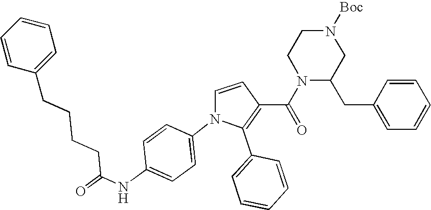

MS (ESI+, m/e) 697 (M+1)

### Reference Example 166

### tert-Butyl 3-benzyl-4-{[1-[2-(pentanoylamino)phenyl]-2-phenyl-1H-pyrrol-3-yl}carbonyl)piperazine-1-carboxylate

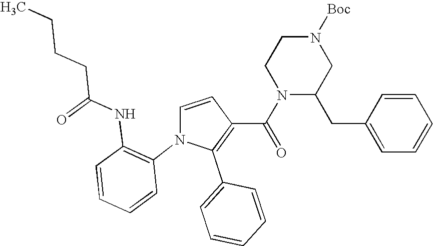

To a solution of ice-cooled tert-butyl 4-{[1-(2-aminophenyl)-2-phenyl-1H-pyrrol-3-yl]carbonyl}-3-benzylpiperazine-1-carboxylate (100 mg), triethylamine (28 mg) and dichloromethane (3 ml), was added pentanoyl chloride (25 mg). After stirring at 0° C. for 1 hr, the mixture was poured into water and extracted with chloroform. The extract was dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (80 mg) as an amorphous solid.

MS (ESI+, m/e) 621 (M+1)

In the same manner as in Reference Example 166, the following compounds (Reference Examples 167 to 169) were obtained.

### Reference Example 167

### tert-Butyl 3-benzyl-4-({1-[2-(butyrylamino)phenyl]-2-phenyl-1H-pyrrol-3-yl}carbonyl)piperazine-1-carboxylate

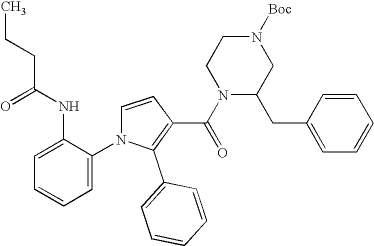

MS (ESI+, m/e) 607 (M+1)

### Reference Example 168

### tert-Butyl 3-benzyl-4-({1-[2-(hexanoylamino)phenyl]-2-phenyl-1H-pyrrol-3-yl}carbonyl)piperazine-1-carboxylate

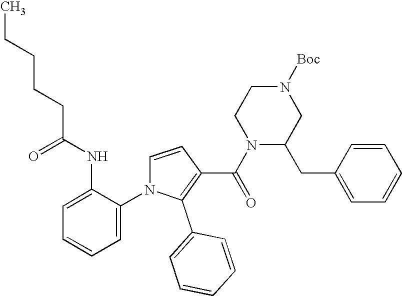

MS (ESI+, m/e) 635 (M+1)

### Reference Example 169

### tert-Butyl 3-benzyl-4-[(1-{2-[(5-ethoxy-5-oxopentanoyl)amino]phenyl}-2-phenyl-1H-pyrrol-3-yl)carbonyl]piperazine-1-carboxylate

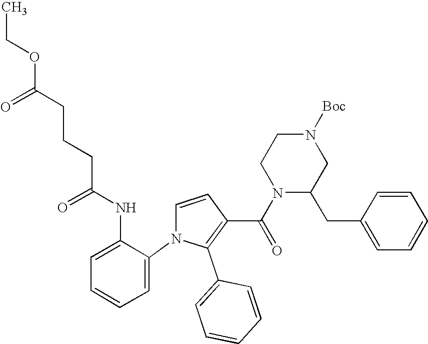

MS (ESI+, m/e) 679 (M+1)

### Reference Example 170

### tert-Butyl (3R)-3-benzyl-4-{[2-(3-hydroxyphenyl)-5-methyl-1-phenyl-1H-pyrrol-3-yl]carbonyl}piperazine-1-carboxylate

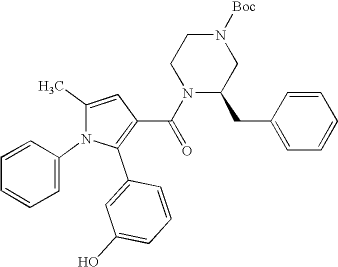

tert-Butyl (3R)-3-benzyl-4-({2-[3-(benzyloxy)phenyl]-5-methyl-1-phenyl-1H-pyrrol-3-yl}carbonyl)piperazine-1-carboxylate (5.08 g) was dissolved in methanol (130 ml), 10% palladium on carbon (containing 50% water, 2.3 g) was added thereto, and the mixture was subjected to catalytic hydrogenation at room temperature and atmospheric pressure for 3 hr. The catalyst was filtered off, and the filtrate was concentrated in vacuo. The residue was subjected to silica gel column chromatography, and the fraction eluted with ethyl acetate-hexane (1:1) was concentrated in vacuo to give the desired product (4.29 g) as an amorphous solid.

MS (ESI+, m/e) 552 (M+1)

### Reference Example 171

### tert-Butyl 3-benzyl-4-{[5-(4-fluorophenyl)-1-(2-hydroxyphenyl)-1H-pyrazol-4-yl]carbonyl}piperazine-1-carboxylate

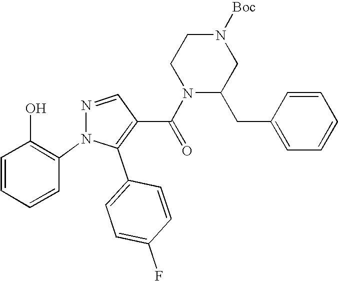

tert-Butyl 3-benzyl-4-{[1-[2-(benzyloxy)phenyl]-5-(4-fluorophenyl)-1H-pyrazol-4-yl]carbonyl}piperazine-1-carboxylate (1.1 g) was dissolved in ethanol (10 ml), 10% palladium on carbon (containing 50% water, 200 mg) was added thereto, and the mixture was subjected to catalytic hydrogenation at room temperature and atmospheric pressure for 15 hr. The catalyst was filtered off, and the filtrate was concentrated in vacuo to give the desired product (765 mg) as an amorphous solid.

MS (ESI+, m/e) 557 (M+1).

In the same manner as in Reference Example 171, the following compounds (Reference Examples 172 to 175) were obtained.

### Reference Example 172

### tert-Butyl 3-benzyl-4-{(1-(3-hydroxyphenyl)-5-phenyl-1H-1,2,3-triazol-4-yl]carbonyl}piperazine-1-carboxylate

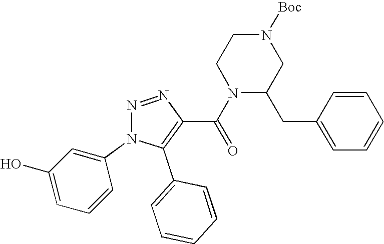

MS (ESI+, m/e) 540 (M+1)

### Reference Example 173

### tert-Butyl 3-benzyl-4-{[1-(4-hydroxyphenyl)-5-phenyl-1H-1,2,3-triazol-4-yl]carbonyl}piperazine-1-carboxylate

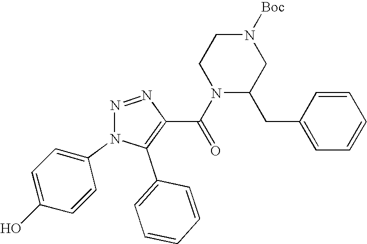

MS (ESI+, m/e) 540 (M+1)

### Reference Example 174

### tert-Butyl 3-benzyl-4-{[1-(2-hydroxyphenyl)-5-phenyl-1H-1,2,3-triazol-4-yl]carbonyl}piperazine-1-carboxylate

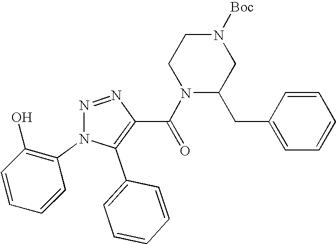

MS (ESI+, m/e) 540 (M+1)

### Reference Example 175

### tert-Butyl (3R)-3-benzyl-4-{[1-(3-hydroxyphenyl)-5-phenyl-1H-1,2,3-triazol-4-yl]carbonyl}piperazine-1-carboxylate

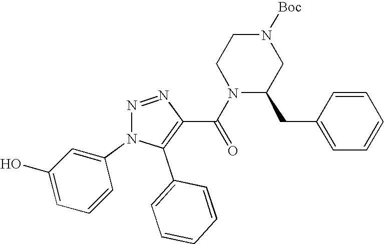

MS (ESI+, m/e) 540 (M+1)

### Reference Example 176

### tert-Butyl 3-benzyl-4-({5-(4,fluorophenyl)-1-[2-(3-methoxypropoxy)phenyl]-1H-pyrazol-4-yl}carbonyl)piperazine-1-carboxylate

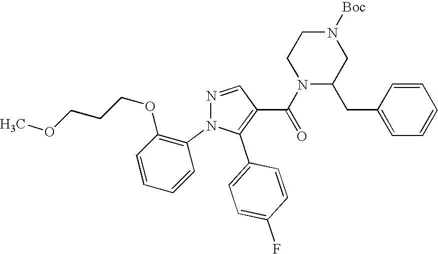

tert-Butyl 3-benzyl-4-{[5-(4-fluorophenyl)-1-(2-hydroxyphenyl)-1H-pyrazol-4-yl]carbonyl}piperazine-1-carboxylate (223 mg), 1-bromo-3-methoxypropane (67 mg) and potassium carbonate (61 mg) were suspended in DMF (2 ml), and the suspension was stirred at 110° C. for 2 hr. Then, the mixture was poured into water and extracted with ethyl acetate. The extract was washed successively with water and brine and dried over anhydrous magnesium sulfate, and then the solvent was evaporated in vacuo. The residue was subjected to basic silica gel column chromatography, and the target fraction was concentrated in vacuo to give the desired product (204 mg) as an amorphous solid.

MS (ESI+, m/e) 629 (M+1)

In the same manner as in Reference Example 176, the following compounds (Reference Examples 177 to 180) were obtained.

### Reference Example 177

### tert-Butyl 3-benzyl-4-[(5-(4-fluorophenyl)-1-{2-[(5-phenylpentyl)oxy]phenyl}-1H-pyrazol-4-yl)carbonyl]piperazine-1-carboxylate

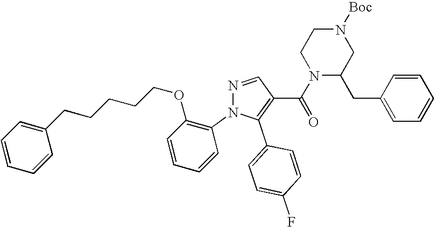

MS (ESI+, m/e) 703 (M+1)

### Reference Example 178

### tert-Butyl 3-benzyl-4-[(5-(4-fluorophenyl)-1-{2-[(6-phenylhexyl)oxy]phenyl}-1H-pyrazol-4-yl)carbonyl]piperazine-1-carboxylate

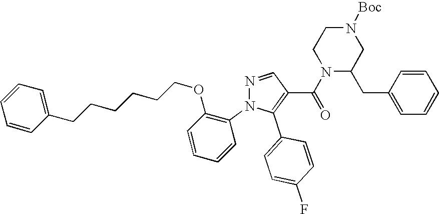

MS (ESI+, m/e) 717 (M+1)

### Reference Example 179

### tert-Butyl 3-benzyl-4-({1-[3-(3-methoxypropoxy)phenyl]-5-phenyl-1H-1,2,3-triazol-4-yl}carbonyl)piperazine-1-carboxylate

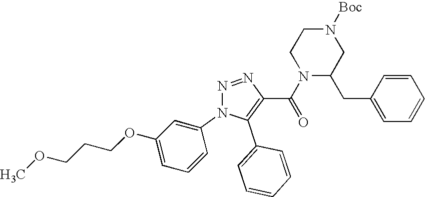

MS (ESI+, m/e) 612 (M+1)

### Reference Example 180

### tert-Butyl (3R)-3-benzyl-4-[(1-{3-[(1,1-dioxidetetrahydro-2H-thiopyran-4-yl)oxy]phenyl}-5-phenyl-1H-1,2,3-triazol-4-yl)carbonyl]piperazine-1-carboxylate

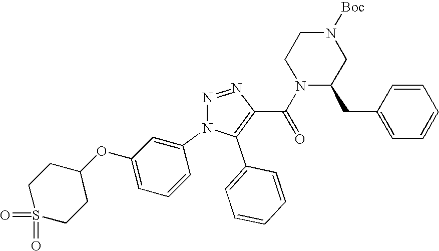

MS (ESI+, m/e) 672 (M+1)

### Reference Example 181

### tert-Butyl (3R)-3-benzyl-4-[(1-{3-[2-(1,1-dioxidothiomorpholino)ethoxy]phenyl)-5-phenyl-1H-1,2,3-triazol-4-yl)carbonyl]piperazine-1-carboxylate

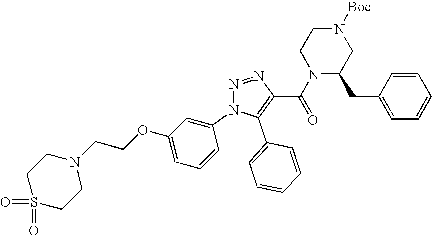

tert-Butyl (3R)-3-benzyl-4-{[1-(3-hydroxyphenyl)-5-phenyl-1H-1,2,3-triazol-4-yl]carbonyl}piperazine-1-carboxylate (270 mg), 2-(1,1-dioxidothiomorpholino)ethanol (134 mg) and triphenylphosphine (197 mg) were dissolved in toluene (5 ml). DEAD (a 40% toluene solution, 327 mg) was added thereto, and the mixture was stirred at room temperature for 15 hr. The solvent was evaporated in vacuo, and the residue was dissolved in ethyl acetate (20 ml). The reaction mixture was washed successively with a 10% aqueous citric acid solution, a 6% aqueous sodium bicarbonate solution and brine and dried over anhydrous magnesium sulfate. Then, the solvent was evaporated in vacuo, the residue was subjected to basic silica gel column chromatography, and the target fraction was concentrated in vacuo to give the desired product (280 mg) as an amorphous solid.

MS (ESI+, m/e) 701 (M+1)

### Reference Example 182

### N-Butyl-3-{3-[(2R)-(2,4-dibenzylpiperazin-1-yl)carbonyl]-5-methyl-2-phenyl-1H-pyrrol-1-yl)-N-methylaniline

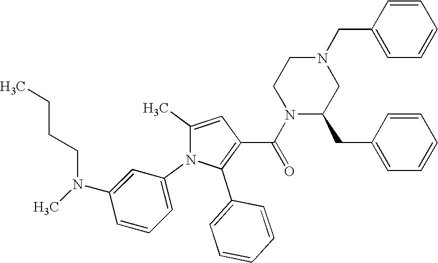

2,4-Dibenzyl-1-{[1-(3-bromophenyl)-5-methyl-2-phenyl-1H-pyrrol-3-yl]carbonyl)piperazine (200 mg), BINAP (19 mg), sodium tert-butoxide (48 mg), and Pd₂(dba)3 (10 mg) were mixed with toluene (3 ml) and N-Methyl-N-butylamine (32 mg) under argon atmosphere. After stirring at 90° C. for 12 hr, the mixture was diluted with ethyl acetate, washed with water and dried over anhydrous sodium sulfate, and then the solvent was evaporated in vacuo. The residue was subjected to silica gel column chromatography, and the fraction eluted with ethyl acetate-hexane (1:1) was concentrated in vacuo to give the desired product (140 mg) as an amorphous solid.

MS (ESI+, m/e) 611 (M+1)

In the same manner as in Reference Example 182, the following compounds (Reference Examples 183 to 190) were obtained.

### Reference Example 183

N-Butyl-3-(3-{[(2R)-2,4-dibenzylpiperazin-1-yl]carbonyl}-5-methyl-2-phenyl-1H-pyrrol-1-yl)aniline

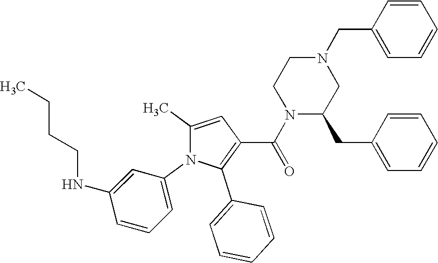

MS (ESI+, m/e) 597 (M+1).

### Reference Example 184

### 4-[3-(3-{[(2R)-2,4-Dibenzylpiperazin-1-yl]carbonyl}-5-methyl-2-phenyl-1H-pyrrol-1-yl)phenyl]morpholine

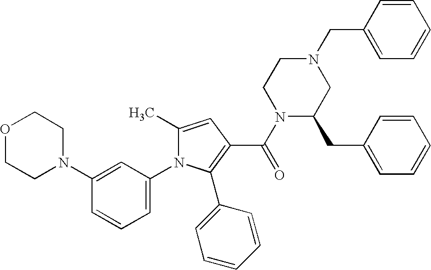

MS (ESI+, m/e) 611 (M+1)

### Reference Example 185

### (2R)-2,4-Dibenzyl-1-{[5-methyl-2-phenyl-1-(3-(piperidin-1-yl)phenyl)-1H-pyrrol-3-yl]carbonyl}piperazine

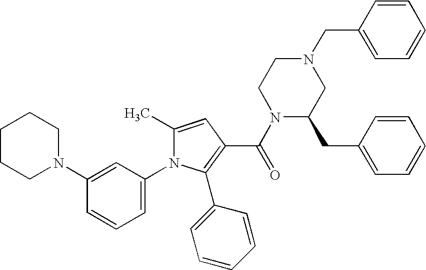

MS (ESI+, m/e) 609 (M+1)

### Reference Example 186

### (2R)-2,4-Dibenzyl-1-({5-methyl-1-[3-(4-methylpiperazin-1-yl)phenyl]-2-phenyl-1H-pyrrol-3-yl}carbonyl)piperazine

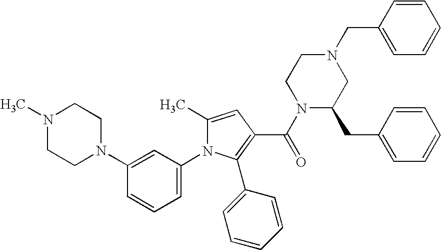

MS (ESI+, m/e) 624 (M+1)

### Reference Example 187

### (2R)-1-({1-[3-(4-Acetylpiperazin-1-yl)phenyl]-5-methyl-2-phenyl-1H-pyrrol-3-yl}carbonyl)-2,4-dibenzylpiperazine

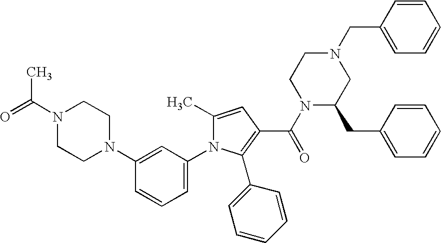

MS (ESI+, m/e) 652 (M+1)

### Reference Example 188

### tert-Butyl 4-[3-(3-{[(2R)-2,4-dibenzylpiperazin-1-yl]carbonyl}-5-methyl-2-phenyl-1H-pyrrol-1-yl)phenyl]piperazine-1-carboxylate

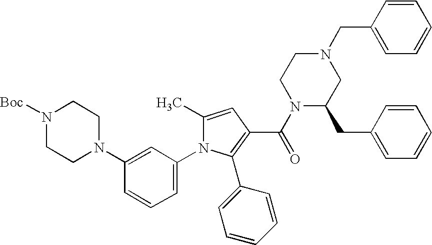

MS (ESI+, m/e) 710 (M+1)

### Reference Example 189

### Ethyl 1-(3-(3-{[(2R)-2,4-dibenzylpiperazin-1-yl]carbonyl}-5-methyl-2-phenyl-1H-pyrrol-1-yl)phenyl]piperidine-4-carboxylate

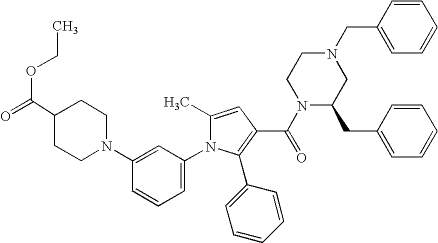

MS (ESI+, m/e) 681 (M+1)

### Reference Example 190

### 4-[3-(3-{[(2R)-2,4-Dibenzylpiperazin-1-yl]carbonyl}-5-methyl-2-phenyl-1H-pyrrol-1-yl)phenyl]thiomorpholine

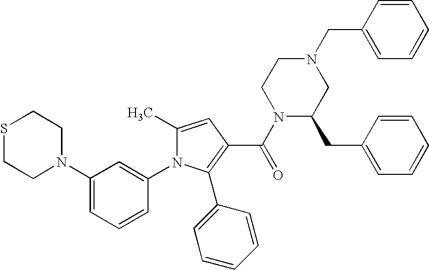

MS (ESI+, m/e) 627 (M+1)

### Reference Example 191

### tert-Butyl (3R)-3-benzyl-4-({5-methyl-1-[3-(1-oxidothiomorpholino)phenyl]-2-phenyl-1H-pyrrol-3-yl}carbonyl)piperazine-1-carboxylate

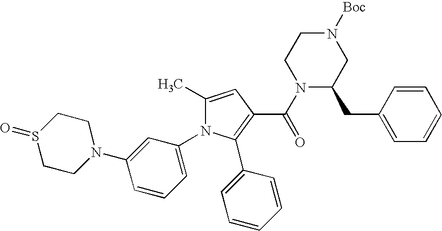

tert-Butyl (3R)-3-benzyl-4-{[1-(3-bromophenyl)-5-methyl-2-phenyl-1H-pyrrol-3-yl]carbonyl}piperazine-1-carboxylate (180 mg), BINAP (28 mg), sodium tert-butoxide (225 mg), Pd₂(dba)₃ (13 mg), and thiomorpholine 1-oxide hydrochloride (30.0 mg) were mixed with 1,4-dioxane (3 ml) under argon atmosphere. After stirring at 80° C. for 12 hr, the mixture was diluted with ethyl acetate, washed with water and dried over anhydrous sodium sulfate. The solvent was evaporated in vacuo, the residue was subjected to silica gel column chromatography, and the fraction eluted with ethyl acetate was concentrated in vacuo to give the desired product (180 mg) as an amorphous solid.

MS (ESI+, m/e) 653 (M+1)

In the same manner as in Reference Example 191, the following compound (Reference Example 192) was obtained using thiomorpholine 1,1-dioxide hydrochloride.

### Reference Example 192

### tert-Butyl (3R)-3-benzyl-4-({1-[3-(1,1-dioxidothiomorpholino)phenyl]-5-methyl-2-phenyl-1H-pyrrol-3-yl}carbonyl)piperazine-1-carboxylate

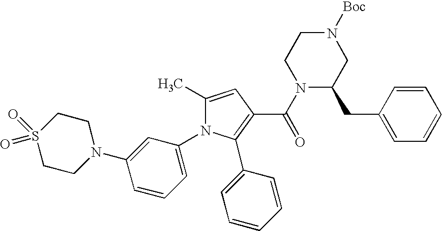

MS (ESI+, m/e) 669 (M+1)

### Reference Example 193

### 4-[3-(3-{[(2R)-2,4-Dibenzylpiperazin-1-yl]carbonyl}-5-methyl-2-phenyl-1H-pyrrol-1-yl)phenyl]piperazin-2-one

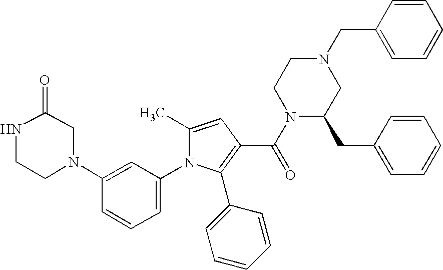

(2R)-2,4-Dibenzyl-1-{[1-(3-bromophenyl)-5-methyl-2-phenyl-1H-pyrrol-3-yl]carbonyl}piperazine (150 mg), dppf (11 mg), 2-piperazinone (75 mg), sodium tert-butoxide (49 mg) and [1,1′-bis(diphenylphosphino)ferrocene]dichloropalladium (II) were suspended in 1,4-dioxane (2.5 ml) under argon atmosphere, and the suspension was stirred at 110° C. for 72 hr. The reaction mixture was poured into water and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-methanol (1:1) was concentrated in vacuo to give the desired product (30 mg) as an amorphous solid.

MS (ESI+, m/e) 624 (M+1)

### Reference Example 194

### 1-[3-(3-{[(2R)-2,4-Dibenzylpiperazin-1-yl]carbonyl}-5-methyl-2-phenyl-1H-pyrrol-1-yl)phenyl]piperidine-4-carboxylic acid

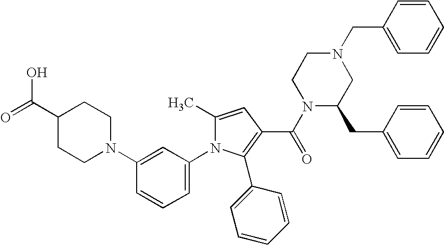

To a solution of ethyl 1-(3-(3-{[(2R)-2,4-dibenzylpiperazin-1-yl]carbonyl}-5-methyl-2-phenyl-1H-pyrrol-1-yl)phenyl]piperidine-4-carboxylate (1.0 g) in ethanol (20 ml), was added a 1 N aqueous sodium hydroxide solution (30 ml). After stirring at room temperature for 30 min and at 60° C. for 30 min, the reaction mixture was concentrated in vacuo, neutralized by adding 2 N hydrochloric acid to the remaining aqueous solution and then extracted with ethyl acetate. The extract was washed with brine and dried over anhydrous sodium sulfate. The solvent was evaporated in vacuo to give the desired product (830 mg).

MS (ESI+, m/e) 653 (M+1)

### Reference Example 195

### {1-[3-(3-{[(2R)-2,4-Dibenzylpiperazin-1-yl]carbonyl}-5-methyl-2-phenyl-1H-pyrrol-1-yl)phenyl]piperidin-4-yl}methanol

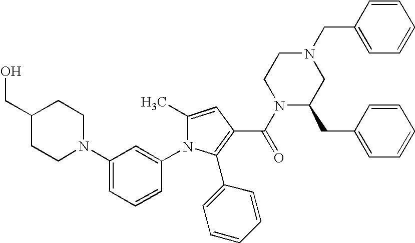

A suspension of sodium borohydride (133 mg), THF (1.5 ml) and ethanol (1.5 ml) was ice-cooled, and calcium carbonate (200 mg) was added thereto. After stirring at 0° C. for 30 min, a solution of ethyl 1-[3-(3-{[(2R)-2,4-dibenzylpiperazin-1-yl]carbonyl}-5-methyl-2-phenyl-1H-pyrrol-1-yl)phenyl]piperidine-4-carboxylate (150 mg) in THF (1.5 ml) was added thereto. The reaction mixture was stirred at 0° C. for 2 hr and at room temperature for 2 hr, and ethyl acetate (20 ml) was gradually added thereto. The mixture was washed successively with water and, brine and dried over anhydrous magnesium sulfate. Then, the solvent was evaporated in vacuo to give the desired product (136 mg) as an amorphous solid.

MS (ESI+, m/e) 639 (M+1) .

### Reference Example 196

### {4-[3-{[(2R)-2,4-Dibenzylpiperazin-1-yl]carbonyl}-1-(2,3-dimethoxyphenyl)-5-methyl-1H-pyrrol-2-yl]phenoxy}acetic acid

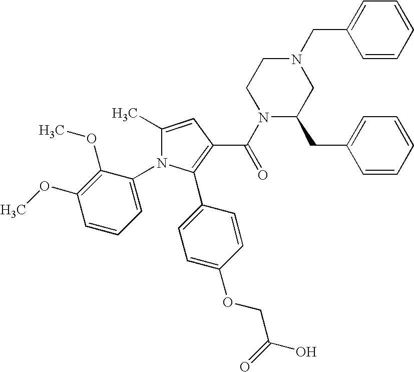

Ethyl {4-[3-{[(2R)-2,4-dibenzylpiperazin-1-yl]carbonyl}-1-(2,3-dimethoxyphenyl)-5-methyl-1H-pyrrol-2-yl]phenoxy}acetate (844 mg) was dissolved in ethanol (20 ml). A 2 N aqueous lithium hydroxide solution (13 ml) was added thereto and stirred at room temperature for 2 hr. The reaction mixture was poured into water, and the reaction mixture was neutralized with 2 N hydrochloric acid, then saturated with sodium chloride and extracted with ethyl acetate. The extract was washed with brine and dried over anhydrous magnesium sulfate. Then, the solvent was evaporated in vacuo, and the crystals were collected by filtration to give the desired product (765 mg).

MS (ESI+, m/e) 660 (M+1)

In the same manner as in Reference Example 196, the following compounds (Reference Examples 197 to 198) were obtained.

### Reference Example 197

### 3-(3-{[(2R)-2,4-Dibenzylpiperazin-1-yl]carbonyl}-5-methyl-2-phenyl-1H-pyrrol-1-yl)-4,5-dimethoxybenzoic acid

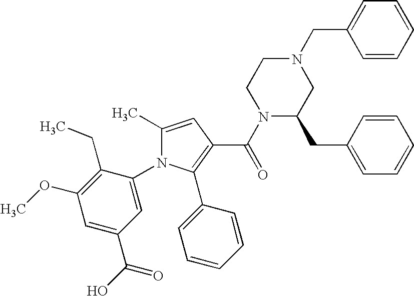

MS (ESI+, m/e) 630 (M+1)

### Reference Example 198

### 3-[3-(3-{[(2R)-2,4-Dibenzylpiperazin-1-yl]carbonyl}-5-methyl-2-phenyl-1H-pyrrol-1-yl)-4,5-dimethoxyphenyl]propanoic acid

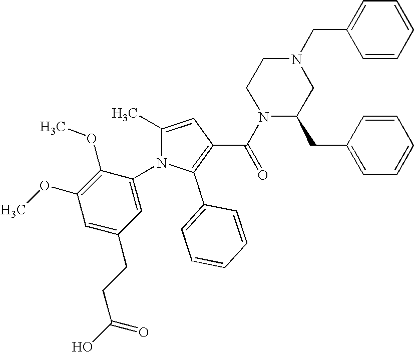

MS (ESI+, m/e) 658 (M+1)

### Reference Example 199

### 5-{[2-(3-{[2-Benzyl-4-(tert-butoxycarbonyl)piperazin-1-yl]carbonyl}-2-phenyl-1H-pyrrol-1-yl)phenyl]amino}-5-oxopentanoic acid

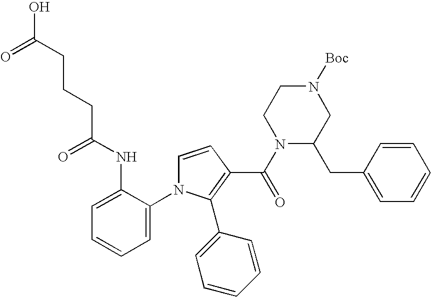

To a solution of tert-butyl 3-benzyl-4-[(1-{2-[(5-ethoxy-5-oxopentanoyl)amino]phenyl}-2-phenyl-1H-pyrrol-3-yl)carbonyl]piperazine-1-carboxylate (120 mg) in ethanol (2 ml), was added a 2 N aqueous sodium hydroxide solution (2 ml). After stirring at room temperature for 2 hr, the reaction mixture was concentrated in vacuo, weakly acidified (pH 3) by adding 2 N hydrochloric acid to the remaining aqueous solution, and extracted with ethyl acetate. The extract was washed with brine and dried over anhydrous sodium sulfate. Then, the solvent was evaporated in vacuo, and the residue was dried in vacuo to give the desired product (100 mg).

¹H-NMR (CDCl₃) δ 1.43 (9H, s), 1.37-1.50 (2H, m), 1.70-1.86 (2H, m), 2.16-5.04 (12H, m), 5.90 (1H, br s), 6.37-7.61 (15H, m), 7.99-8.05 (1H, m)

### Reference Example 200

### 4-(tert-Butoxycarbonyl)-1-[(5-methyl-1,2-diphenyl-1H-pyrrol-3-yl)carbonyl]piperazine-2-carboxylic acid

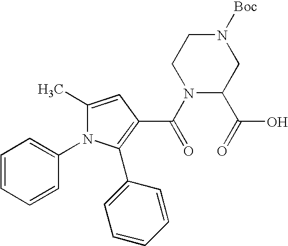

3-Ethyl 1-tert-butyl 4-[(5-methyl-1,2-diphenyl-1H-pyrrol-3-yl)carbonyl]piperazine-1,3-dicarboxylate (518 mg) was dissolved in ethanol (5 ml), lithium hydroxide monohydrate (252 mg) was added thereto, and the mixture was stirred at room temperature for 15 hr. The solvent was then evaporated in vacuo, and adjusted to pH 6 by adding 1 N hydrochloric acid to the residue. The liberated oil was extracted with chloroform, and the extract was washed with brine and dried over anhydrous magnesium sulfate. Then, the solvent was evaporated in vacuo to give the desired product (480 mg) as an amorphous solid.

MS (ESI+, m/e) 490 (M+1)

### Reference Example 201

### 4-({4-(tert-Butoxycarbonyl)-1-[(5-methyl-1,2-diphenyl-1H-pyrrol-3-yl)carbonyl]piperazin-2-yl}methoxy)benzoic acid

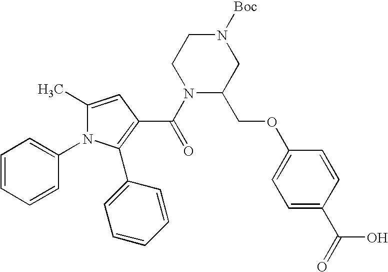

tert-Butyl 3-{[4-(methoxycarbonyl)phenoxy]methyl}-4-[(5-methyl-1,2-diphenyl-1H-pyrrol-3-yl)carbonyl}piperazine-1-carboxylate (200 mg) was dissolved in methanol (2 ml). Potassium hydroxide (56 mg) was added thereto, and the mixture was heated under reflux for 1 hr. The solvent was evaporated in vacuo, and adjusted to pH 5 by adding a 10% aqueous citric acid solution to the residue. The liberated oil was extracted with chloroform, and the extract was washed with brine and dried over anhydrous magnesium sulfate. Then, the solvent was evaporated in vacuo to give the desired product (160 mg) as an amorphous solid.

MS (ESI+, m/e) 596 (M+1)

In the same manner as in Reference Example 201, the following compounds (Reference Examples 202 to 206) were obtained.

### Reference Example 202

### [4-({4-(tert-Butoxycarbonyl)-1-[(5-methyl-1,2-diphenyl-1H-pyrrol-3-yl)carbonyl]piperazin-2-yl}methoxy)phenyl]acetic acid

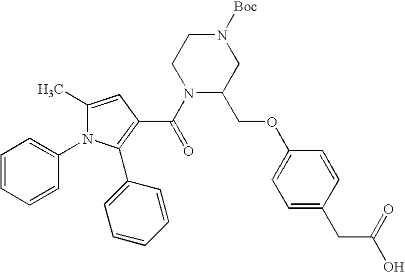

MS (ESI+, m/e) 610 (M+1)

### Reference Example 203

### 3-[4-({4-(tert-Butoxycarbonyl)-1-[(5-methyl-1,2-diphenyl-1H-pyrrol-3-yl)carbonyl]piperazin-2-yl}methoxy)phenyl]propanoic acid

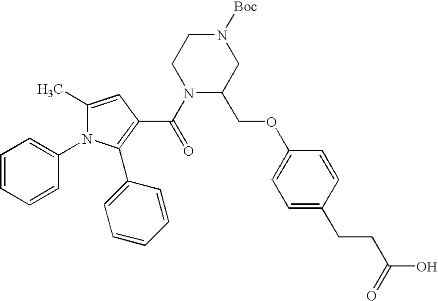

MS (ESI+, m/e) 624 (M+1)

### Reference Example 204

### 3-({4-(tert-Butoxycarbonyl)-1-[(5-methyl-1,2-diphenyl-1H-pyrrol-3-yl)carbonyl]piperazin-2-yl}methoxy)benzoic acid

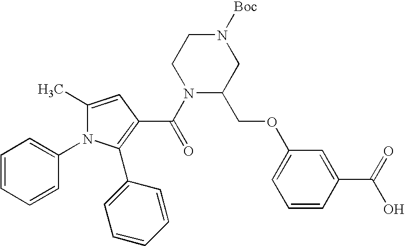

MS (ESI+, m/e) 596 (M+1)

### Reference Example 205

### 4-({4-Benzyl-1-[(5-methyl-1,2-diphenyl-1H-pyrrol-3-yl)carbonyl]piperazin-2-yl}methyl)benzoic acid

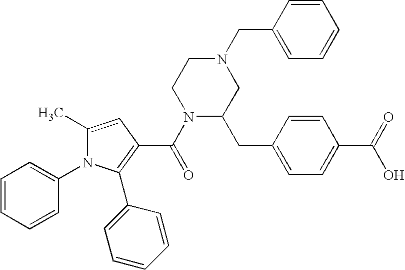

MS (ESI+, m/e) 570 (M+1)

### Reference Example 206

### 4-[(2R)-4-Benzyl-1-{[1-(3-morpholinophenyl)-5-phenyl-1H-imidazol-4-yl]carbonyl}piperazin-2-yl)methyl]benzoic acid

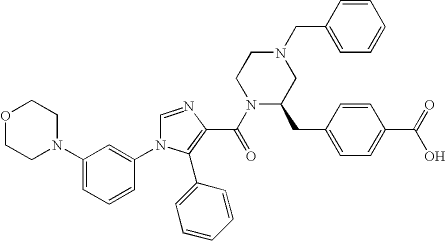

MS (ESI+, m/e) 642 (M+1)

### Reference Example 207

### {4-(3-{((2R)-2,4-Dibenzylpiperazin-1-yl]carbonyl}-1-(2,3-dimethoxyphenyl)-5-methyl-1H-pyrrol-2-yl]phenoxy)acetamide

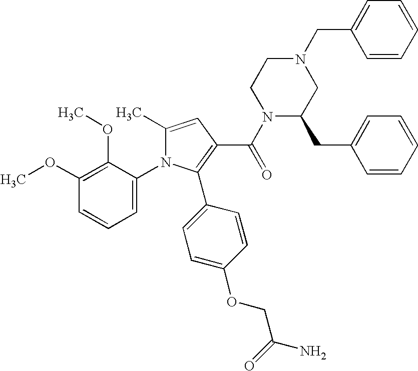

A solution of {4-[3-{[(2R)-2,4-dibenzylpiperzin-1-yl]carbonyl}-1-(2,3-dimethoxyphenyl)-5-methyl-1H-pyrrol-2-yl]phenoxy}acetic acid (400 mg), ammonium salts of HOBt (111 mg), WSC.HCl (139 mg) and DMF (6 ml) was stirred at room temperature for 15 hr. Then, the reaction mixture was poured into a saturated aqueous sodium bicarbonate solution and extracted with ethyl acetate. The extract was washed is successively with water and brine and dried over anhydrous magnesium sulfate, and then the solvent was evaporated in vacuo. The residue was subjected to basic silica gel column chromatography, and the fraction eluted with ethyl acetate was concentrated in vacuo to give the desired product (354 mg) as an amorphous solid.

MS (ESI+, m/e) 659 (M+1)

In the same manner as in Reference Example 207, the following compounds (Reference Examples 208 to 212) were obtained.

### Reference Example 208

### 1-[3-(3-{[(2R)-2,4-Dibenzylpiperazin-1-yl]carbonyl}-5-methyl-2-phenyl-1H-pyrrol-1-yl)phenyl]piperidine-4-carboxamide

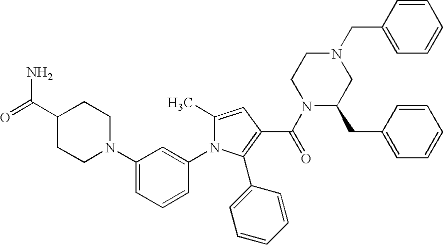

MS (ESI+, m/e) 652 (M+1)

### Reference Example 209

### 3-(3-{[(2R)-2,4-Dibenzylpiperazin-1-yl]carbonyl}-5-methyl-2-phenyl-1H-pyrrol-1-yl)-4,5-dimethoxybenzamide

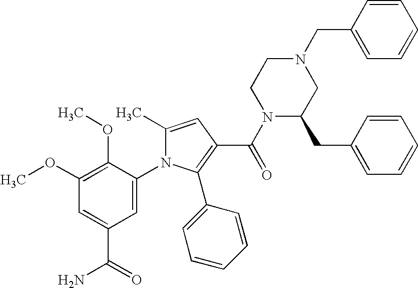

MS (ESI+, m/e) 539 (M+1)

### Reference Example 210

### 3-[3-(3-{[(2R)-2,4-Dibenzylpiperazin-1-yl]carbonyl}-5-methyl-2-phenyl-1H-pyrrol-1-yl)-4,5-dimetboxyphenyl propanamide

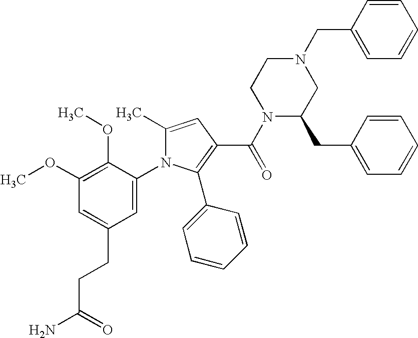

MS (ESI+, m/e) 567 (M+1)

### Reference Example 211

### tert-Butyl 3-{[4-(aminocarbonyl)phenoxy]methyl}-4-[(5-methyl-1,2-diphenyl-1H-pyrrol-3-yl)carbonyl]piperazine-1-carboxylate

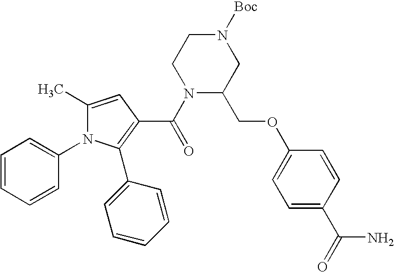

MS (ESI+, m/e) 595 (M+1)

### Reference Example 212

### 4-({4-Benzyl-1-[(5-methyl-1,2-diphenyl-1H-pyrrol-3-yl)carbonyl]piperazin-2-yl}methyl)benzamide

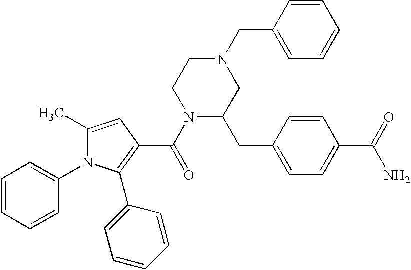

MS (ESI+, m/e) 569 (M+1)

### Reference Example 213

### 1-[3-(3-{[(2R)-2,4-Dibenzylpiperazin-1-yl]carbonyl}-5-methyl-2-phenyl-1H-pyrrol-1-yl)phenyl]-N-(4-hydroxybutyl)piperidine-4-carboxamide

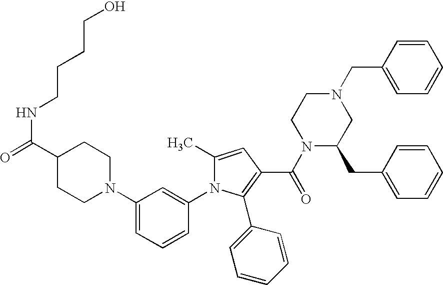

A solution of 1-[3-(3-{[(2R)-2,4-dibenzylpiperazin-1-yl]carbonyl}-5-methyl-2-phenyl-1H-pyrrol-1-yl)phenyl]piperidine-4-carboxylic acid (150 mg), WSC.HCl (66 mg), HOBt (46 mg), 4-aminobutanol (25 mg) and DMF (3 ml) was stirred at room temperature for 12 hr. The reaction mixture was poured into a saturated aqueous sodium bicarbonate solution and extracted with ethyl acetate. The extract was washed with brine and dried over anhydrous sodium sulfate, and then the solvent was evaporated in vacuo. The residue was subjected to basic silica gel column chromatography, and the fraction eluted with ethyl acetate was concentrated in vacuo to give the desired product (140 mg) as an amorphous solid.

MS (ESI+, m/e) 724 (M+1)

In the same manner as in Reference Example 213, the following compounds (Reference Examples 214 to 222) were obtained.

### Reference Example 214

### N-(3-Amino-3-oxopropyl)-1-[3-(3-{[(2R)-2,4-dibenzylpiperazin-1-yl]carbonyl)-5-methyl-2-phenyl-1H-pyrrol-1-yl)phenyl]piperidine-4-carboxamide

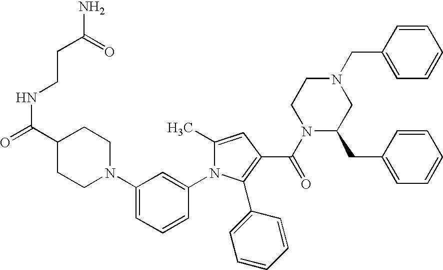

MS (ESI+, m/e) 723 (M+1)

### Reference Example 215

### 1-[3-(3-{[(2R)-2,4-Dibenzylpiperazin-1-yl]carbonyl}-5-methyl-2-phenyl-1H-pyrrol-1-yl)phenyl]-N-(2-hydroxyethyl)piperidine-4-carboxamide

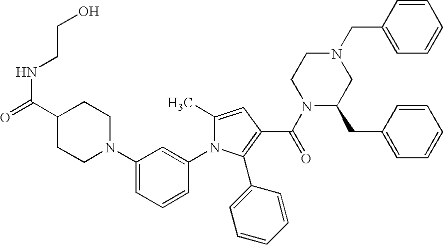

MS (ESI+, m/e) 696 (M+1)

### Reference Example 216

### 3-(3-{[(2R)-2,4-Dibenzylpiperazin-1-yl]carbonyl}-5-methyl-2-phenyl-1H-pyrrol-1-yl)-N-(4-hydroxybutyl)-4,5-dimethoxybenzamide

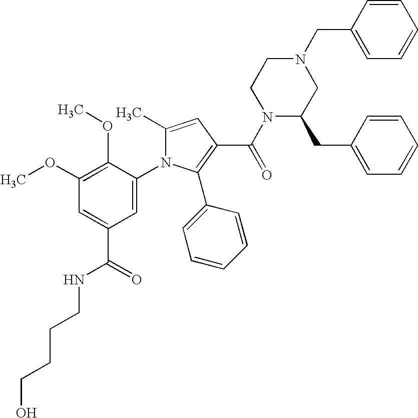

MS (ESI+, m/e) 611 (M+1)

### Reference Example 217

### 3-[3-(3-{[(2R)-2,4-Dibenzylpiperazin-1-yl]carbonyl}-5-methyl-2-phenyl-1H-pyrrol-1-yl)-4,5-dimethoxyphenyl]-N-(4-hydroxybutyl)propanamide

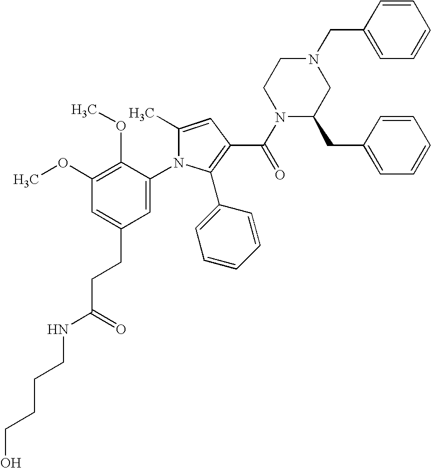

MS (ESI+, m/e) 639 (M+1)

### Reference Example 218

### 4-[3-(3-{[(2R)-2,4-Dibenzylpiperazin-1-yl]carbonyl}-5-methyl-2-phenyl-1H-pyrrol-1-yl)-4,5-dimethoxybenzoyl]morpholine

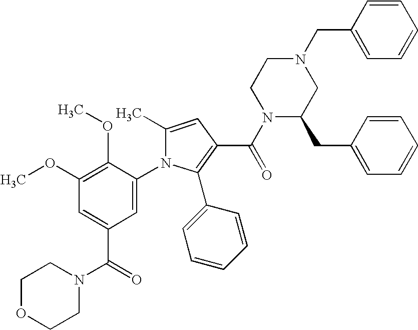

MS (ESI+, m/e) 609 (M+1)

### Reference Example 219

### 4-{3-[3-(3-{[(2R)-2,4-Dibenzylpiperazin-1-yl]carbonyl}-5-methyl-2-phenyl-1H-pyrrol-1-yl)-4,5-dimethoxyphenyl]propanoyl}morpholine

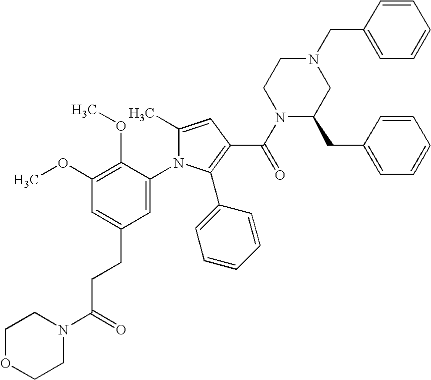

MS (ESI+, m/e) 637 (M+1)

### Reference Example 220

### tert-Butyl 4-[(5-methyl-1,2-diphenyl-1H-pyrrol-3-yl)carbonyl]-3-{[(pyridin-2-yl)amino]carbonyl}piperazine-1-carboxylate

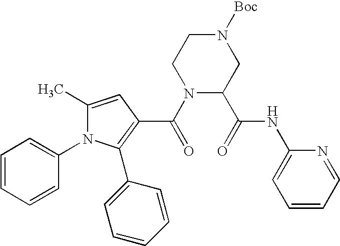

MS (ESI+, m/e) 566 (M+1)

### Reference Example 221

### tert-Butyl 3-[(benzylamino)carbonyl]-4-[(5-methyl-1,2-diphenyl-1H-pyrrol-3-yl)carbonyl]piperazine-1-carboxylate

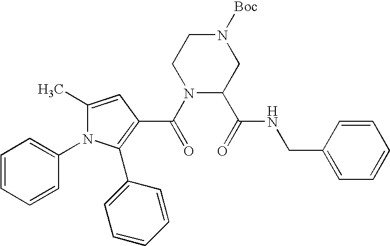

MS (ESI+, m/e) 579 (M+1)

### Reference Example 222

### 4-({4-Benzyl-1-[(5-methyl-1,2-diphenyl-1H-pyrrol-3-yl)carbonyl]piperazin-2-yl}methyl)-N-(2-hydroxyethyl)benzamide

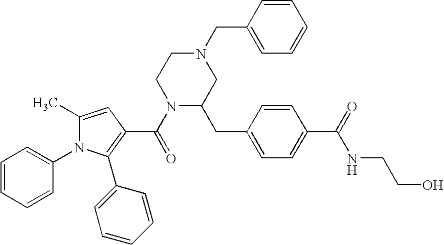

MS (ESI+, m/e) 613 (M+1)

### Reference Example 223

### (2R)-2,4-Dibenzyl-1-{[5-methyl-2-phenyl-1-(3-(piperazin-1-yl)phenyl)-1H-pyrrol-3-yl]carbonyl}piperazine

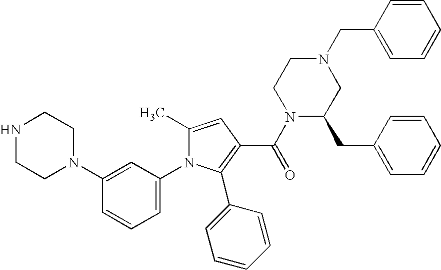

To a solution of tert-butyl 4-[3-(3-{[(2R)-2,4-dibenzylpiperazin-1-yl]carbonyl)-5-methyl-2-phenyl-1H-pyrrol-1-yl)phenyl]piperazine-1-carboxylate (200 mg) in chloroform (1 ml), was added TFA (1 ml) and stirred at room temperature for 2 hr. Then, the solvent was evaporated in vacuo, and the residue was dissolved in chloroform. The reaction mixture was washed with a saturated aqueous sodium bicarbonate solution and dried over anhydrous sodium sulfate. The solvent was evaporated in vacuo to give the desired product (150 mg) as an amorphous solid.

MS (ESI+, m/e) 610 (M+1)

### Reference Example 224

### N-(3-{4-[3-(3-{[(2R)-2,4-Dibenzylpiperazin-1-yl]carbonyl}-5-methyl-2-phenyl-1H-pyrrol-1-yl)phenyl]piperazin-1-yl}-3-oxopropyl)acetamide

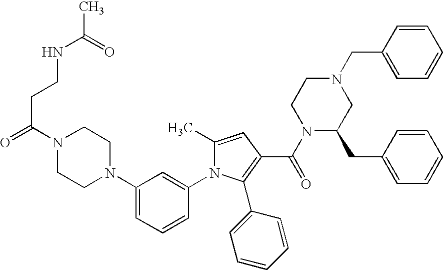

A solution of (2R)-2,4-dibenzyl-1-{(5-methyl-2-phenyl-1-(3-(piperazin-1-yl)phenyl)-1H-pyrrol-3-yl]carbonyl}piperazine (120 mg), N-acetyl-O-alanine (31 mg), WSC.HCl (57 mg), HOBt (39 mg) and DMF (5 ml) was stirred at room temperature for 12 hr. The reaction mixture was poured into a saturated aqueous sodium bicarbonate solution and extracted with ethyl acetate. The extract was washed with brine and dried over anhydrous sodium sulfate, and the solvent was then evaporated in vacuo. The residue was subjected to silica gel column chromatography, and the fraction eluted with ethyl acetate was concentrated in vacuo to give the desired product (70 mg) as an amorphous solid.

MS (ESI+, m/e) 723 (M+1)

### Reference Example 225

### 3-(3-{[(2R)-2,4-Dibenzylpiperazin-1-yl]carbonyl}-5-methyl-2-phenyl-1H-pyrrol-1-yl)benzonitrile

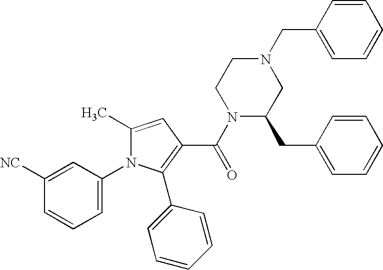

Under an argon atmosphere, a solution of zinc cyanide (70 mg), 2,4-dibenzyl-1-{[1-(3-bromophenyl)-5-methyl-2-phenyl-1H-pyrrol-3-yl]carbonyl}piperazine (300 mg), tetrakis(triphenylphosphine)palladium(0) (58 mg) and MAF (2 ml) was stirred at 80° C. for 4 hr. The reaction mixture was poured into a saturated aqueous sodium bicarbonate solution and extracted with ethyl acetate. The extract was washed with brine and dried over anhydrous sodium sulfate, and then the solvent was evaporated in vacuo. The residue was subjected to silica gel column chromatography, and the fraction eluted with ethyl acetate-hexane (1:1) was concentrated in vacuo to give the desired product (245 mg) as an amorphous solid.

MS (ESI+, m/e) 551 (M+1)

In the same manner as in Reference Example 225, the following compound (Reference Example 226) was obtained.

### Reference Example 226

### tert-Butyl (3R)-3-benzyl-4-{[1-(3-cyanophenyl)-5-methyl-2-phenyl-1H-pyrrol-3-yl]carbonyl}piperazine-1-carboxylate

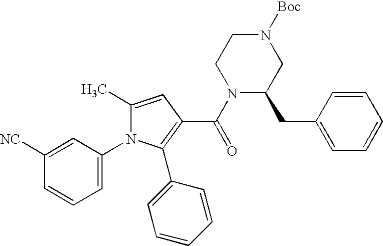

MS (ESI+, m/e) 561 (M+1)

### Reference Example 227

### 3-(3-{[(2R)-2,4-Dibenzylpiperazin-1-yl]carbonyl}-5-methyl-2-phenyl-1H-pyrrol-1-yl)benzamide

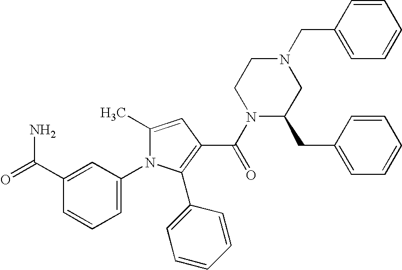

To a solution of 3-(3-{[(2R)-2,4-dibenzylpiperazin-1-yl]carbonyl}-5-methyl-2-phenyl-1H-pyrrol-1-yl)benzonitrile (240 mg) in DMSO (10 ml), was added a 6 N sodium hydroxide solution. (10 ml). The mixture was stirred at 60° C. for 1 hr, and then acidified with 2 N hydrochloric acid. Water and ethyl acetate were added thereto, and the organic layer was washed with brine and dried over anhydrous sodium sulfate. The solvent was evaporated in vacuo, the residue was subjected to basic silica gel column chromatography, and the fraction eluted with ethyl acetate was concentrated in vacuo to give the desired product (220 mg) as an amorphous solid.

MS (ESI+, m/e) 569 (M+1)

### Reference Example 228

### (2R)-2,4-Dibenzyl-1-({5-methyl-2-phenyl-1-[3-(1H-tetrazol-5-yl)phenyl]-1H-pyrrol-3-yl}carbonyl)piperazine

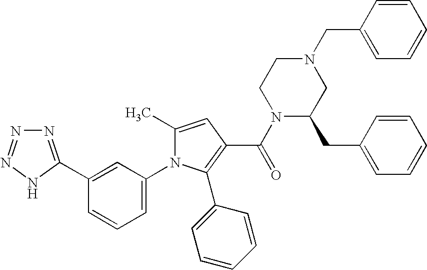

To a solution of 3-(3-{[(2R)-2,4-dibenzylpiperazin-1-yl]carbonyl}-5-methyl-2-phenyl-1H-pyrrol-1-yl)benzonitrile (400 mg) in toluene (10 ml) were added trimethylsilyl azide (84 mg) and dibutyltin oxide (18 mg). The mixture was heated under reflux for 12 hr, and then the solvent was evaporated in vacuo. To the residue were added a saturated aqueous sodium bicarbonate solution and ethyl acetate. The reaction mixture was stirred, and then adjusted to pH 2 to 3 by gradually adding 6 N hydrochloric acid. The organic layer was washed with a saturated aqueous sodium bicarbonate solution and dried over anhydrous sodium sulfate, and the solvent was then evaporated in vacuo. The residue was subjected to silica gel column chromatography, and the fraction eluted with ethyl acetate-methanol (4:1) was concentrated in vacuo to give the desired product (285 mg) as an amorphous solid.

MS (ESI+, m/e) 594 (M+1)

### Reference Example 229

### tert-Butyl 3-benzyl-4-({1-(2-(pentylamino)phenyl]-2-phenyl-1H-pyrrol-3-yl}carbonyl)piperazine-1-carboxylate

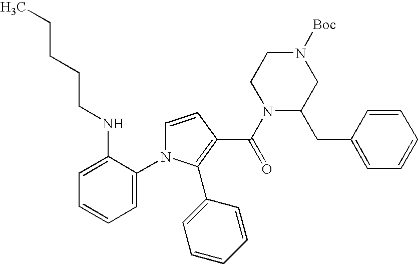

To a solution of tert-butyl 4-{[1-(2-aminophenyl)-2-phenyl-1H-pyrrol-3-yl]carbonyl}-3-benzylpiperazine-1-carboxylate (95 mg) in ethanol (2 ml), was added valeraldehyde (85 mg): The mixture was stirred at room temperature for 2 hr, and then sodium triacetoxyborohydride (225 mg) was added thereto. After further mixing at room temperature for 12 hr, the reaction mixture was poured into a saturated aqueous sodium bicarbonate solution, and the solvent was evaporated in vacuo. The remaining aqueous solution was extracted with ethyl acetate, and the extract was washed with brine and dried over anhydrous sodium sulfate. Then, the solvent was evaporated in vacuo, the residue was subjected to silica gel column chromatography, and the fraction eluted with ethyl acetate-hexane (1:1) was concentrated in vacuo to give the desired product (61 mg) as an amorphous solid.

MS (ESI+, m/e) 607 (M+1)

In the same manner as in Reference Example 229, the following compounds (Reference Examples 230 to 231) were obtained.

### Reference Example 230

### tert-Butyl 3-benzyl-4-({1-[2-(butylamino)phenyl]-2-phenyl-1H-pyrrol-3-yl}carbonyl)piperazine-1-carboxylate

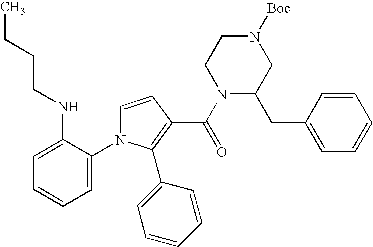

MS (ESI+, m/e) 593 (M+1)

### Reference Example 231

### tert-Butyl 3-benzyl-4-({2-phenyl-1-[2-(propylamino)phenyl]-1H-pyrrol-3-yl}carbonyl)piperazine-1-carboxylate

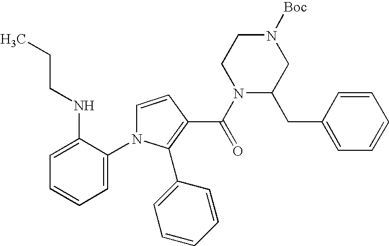

MS (ESI+, m/e) 579 (M+1)

### Reference Example 232

### tert-Butyl (3R)-4-[(1-{2-[(N-acetyl-β-alanyl)amino]phenyl}-2-phenyl-1H-pyrrol-3-yl)carbonyl]-3-benzylpiperazine-1-carboxylate

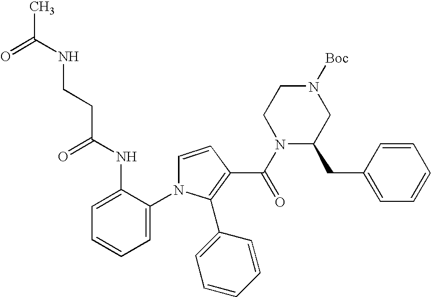

To a solution of N-acetyl-β-alanine (50 mg), which was cooled to −15° C., 4-methylmorpholine (42 mg) and ethyl acetate (2 ml), was added a solution of ethyl chloroformate (40 mg) in ethyl acetate (2 ml). The mixture was stirred at −15° C. for 15 min, and then a solution of tert-butyl (3R)-4-{[1-(2-aminophenyl)-2-phenyl-1H-pyrrol-3-yl]carbonyl}-3-benzylpiperazine-1-carboxylate (200 mg) in ethyl acetate (2 ml) was added thereto. After stirring at −15° C. for 15 min and at room temperature for 2 hr, the mixture was poured into a saturated aqueous sodium bicarbonate solution and extracted with ethyl acetate. The extract was dried over anhydrous sodium sulfate, and the solvent was evaporated in vacuo. The residue was subjected to reverse-phase HPLC analysis (the purification condition is described above), and the target fraction was neutralized with a saturated aqueous sodium bicarbonate solution and extracted with ethyl acetate. The extract was dried over anhydrous sodium sulfate, and the solvent was evaporated in vacuo. The residue was dried in vacuo to give the desired product (56 mg) as an amorphous solid.

MS (ESI+, m/e) 650 (M+1)

### Reference Example 233

### tert-Butyl (3R)-3-benzyl-4-[(1-{2-[(3-methoxypropyl)amino]phenyl}-2-phenyl-1H-pyrrol-3-yl)carbonyl]piperazine-1-carboxylate

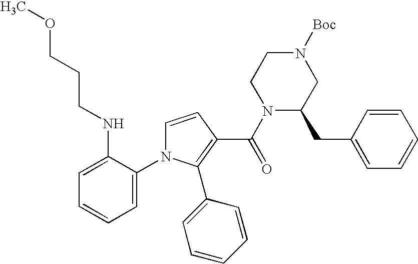

A solution of tert-butyl (3R)-4-{[1-(2-aminophenyl)-2-phenyl-1H-pyrrol-3-yl]carbonyl}-3-benzylpiperazine-1-carboxylate (250 mg), 1-bromo-3-methoxypropane (85 mg), calcium carbonate (56 mg) and DMF (3 ml) was stirred at 100° C. for 2 days. The reaction mixture was poured into an aqueous sodium bicarbonate solution and extracted with ethyl acetate.

The extract was washed with brine and dried over anhydrous sodium sulfate, and then the solvent was evaporated in vacuo. The residue was subjected to silica gel column chromatography, and the fraction eluted with ethyl acetate-hexane (1:1) was concentrated in vacuo to give the desired product (70 mg) as an amorphous solid.

MS (ESI+, m/e) 609 (M+1)

### Reference Example 234

### tert-Butyl (3R)-3-benzyl-4-({1-[5-methoxy-2-(propionylamino)phenyl]-2-phenyl-1H-pyrrol-3-yl}carbonyl)piperazine-1-carboxylate

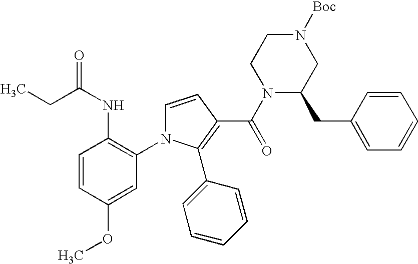

To a solution of tert-butyl (3R)-3-benzyl-4-{[1-(5-methoxy-2-aminophenyl)-2-phenyl-1H-pyrrol-3-yl]carbonyl}piperazine-1-carboxylate (200 mg) in DMA (3 ml), was added propionyl chloride (39 mg) at 0° C. The mixture was stirred at room temperature for 30 min, and then poured into a saturated aqueous sodium bicarbonate solution. The suspension was filtered, and the crystals were washed with water, and then dissolved in ethyl acetate. The reaction mixture was dried over anhydrous sodium sulfate, and the solvent was evaporated in vacuo. The residue was subjected to basic silica gel column chromatography, and the fraction eluted with ethyl acetate-hexane (1:1) was concentrated in vacuo to give the desired product (210 mg) as an amorphous solid.

MS (ESI+, m/e) 623 (M+1)

### Reference Example 235

### tert-Butyl (3R)-3-benzyl-4-{[1-(2-hydroxyphenyl)-5-methyl-2-phenyl-1H-pyrrol-3-yl]carbonyl}piperazine-1-carboxylate

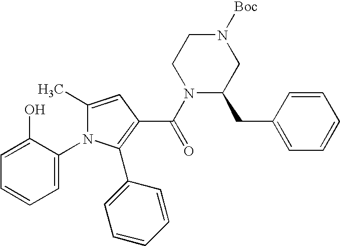

To a solution of 2-(3-{[(2R)-2-benzylpiperazin-1-yl]carbonyl}-5-methyl-2-phenyl-1H-pyrrol-1-yl)phenol (the free compound of Example 51 described hereinafter, 160 mg) in dichloromethane (1.6 ml), was added a solution of di-tert-butyl dicarbonate (72 mg) in dichloromethane (1 ml). The mixture was stirred at room temperature for 1 hr, and then the solvent was evaporated in vacuo. The residue was subjected to silica gel column chromatography, and the fraction eluted with ethyl acetate-hexane (1:1) was concentrated in vacuo to give the desired product (170 mg) as an amorphous solid.

MS (ESI+, m/e) 552 (M+1)

### Reference Example 236

### tert-Butyl (3R)-3-benzyl-4-{(1-(2-methoxyphenyl)-5-methyl-2-phenyl-1H-pyrrol-3-yl]carbonyl}piperazine-1-carboxylate

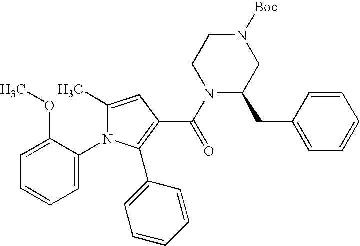

To a suspension of tert-butyl (3R)-3-benzyl-4-{[1-(2-hydroxyphenyl)-5-methyl-2-phenyl-1H-pyrrol-3-yl]carbonyl}piperazine-1-carboxylate (120 mg), potassium carbonate (60 mg) and DMF (5 ml), was added methyl iodide (48 mg). The mixture was stirred at room temperature for 12 hr, then poured into water and extracted with ethyl acetate. The extract was washed with brine and dried over anhydrous magnesium sulfate, and then the solvent was evaporated in vacuo. The residue was subjected to silica gel column chromatography, and the fraction eluted with ethyl acetate-hexane (1:1) was concentrated in vacuo to give the desired product (110 mg) as an oil.

MS (ESI+, m/e) 566 (M+1)

### Reference Example 237

### tert-Butyl (3R)-3-benzyl-4-[(1-{3-[(1,1-dioxidotetrahydro-2H-thiopyran-4-yl)oxy]phenyl}-5-methyl-2-phenyl-1H-pyrrol-3-yl)carbonyl]piperazine-1-carboxylate

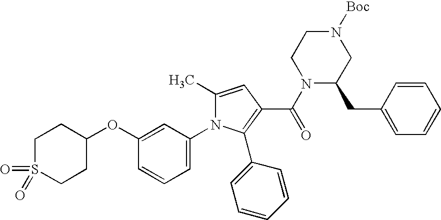

To a suspension of tert-butyl (3R)-3-benzyl-4-{[1-(3-hydroxyphenyl)-5-methyl-2-phenyl-1H-pyrrol-3-yl]carbonyl}piperazine-1-carboxylate (200 mg), potassium carbonate (150 mg) and DMF (5 ml), was added 1,1-dioxidotetrahydro-2H-thiopyran-4-yl 4-methylbenzenesulfonate (274 mg). The mixture was stirred at 90° C. for 12 hr, then poured into water and extracted with ethyl acetate. The extract was washed with brine and dried over anhydrous magnesium sulfate, and then the solvent was evaporated in vacuo. The residue was subjected to silica gel column chromatography, and the fraction eluted with ethyl acetate was concentrated in vacuo to give the desired product (225 mg) as an amorphous solid.

MS (ESI+, m/e) 684 (M+1)

In the same manner as in Reference Example 237, the following compound (Reference Example 238) was obtained.

### Reference Example 238

### tert-Butyl (3R)-3-benzyl-4-[(5-methyl-1-{3-[3-(methylsulfonyl)propoxy]phenyl}-2-phenyl-1H-pyrrol-3-yl)carbonyl]piperazine-1-carboxylate

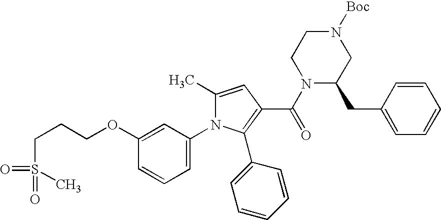

MS (ESI+, m/e) 672 (M+1)

### Reference Example 239

### tert-Butyl (3R)-3-benzyl-4-({1-[3-(2-hydroxy-2-methylpropoxy)phenyl]-5-methyl-2-phenyl-1H-pyrrol-3-yl}carbonyl)piperazine-1-carboxylate

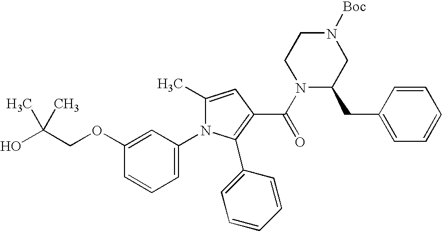

To a suspension of tert-butyl (3R)-3-benzyl-4-{[1-(3-hydroxyphenyl)-5-methyl-2-phenyl-1H-pyrrol-3-yl]carbonyl}piperazine-1-carboxylate (200 mg), potassium carbonate (150 mg) and DMF (5 ml), was added 2,2-dimethyloxirane (39 mg). The mixture was stirred at 100° C. for 12 hr, then poured into water and extracted with ethyl acetate. The extract was washed with brine and dried over anhydrous magnesium sulfate, and then the solvent was evaporated in vacuo. The residue was subjected to silica gel column chromatography, and the fraction eluted with ethyl acetate was concentrated in vacuo to give the desired product (71 mg) as an amorphous solid.

MS (ESI+, m/e) 624 (M+1)

### Reference Example 240

### tert-Butyl (3R)-3-benzyl-4-[(5-methyl-1-{3-[2-(2-oxoimidazolidin-1-yl) ethoxy]phenyl}-2-phenyl-1H-pyrrol-3-yl)carbonyl]piperazine-1-carboxylate

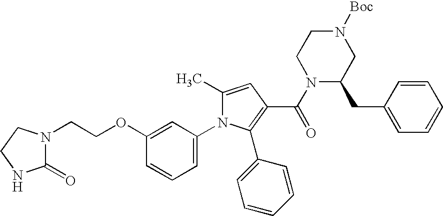

To a solution of tert-butyl (3R)-3-benzyl-4-{[1-(3-hydroxyphenyl)-5-methyl-2-phenyl-1H-pyrrol-3-yl]carbonyl}piperazine-1-carboxylate (200 mg), DEAD (135 mg), 1-(2-hydroxyethyl)imidazolidin-2-one (70 mg) and THF (5 ml), was added triphenylphosphine (143 mg). The mixture was stirred at room temperature for 12 hr, then poured into water and extracted with ethyl acetate. The extract was dried over anhydrous magnesium sulfate, and then the solvent was evaporated in vacuo. The residue was subjected to silica gel column chromatography, and the fraction eluted with ethyl acetate was concentrated in vacuo to give the desired product (220 mg) as an amorphous solid.

MS (ESI+, m/e) 664 (M+1)

### Reference Example 241

### tert-Butyl [4-({4-benzyl-1-[(5-methyl-1,2-diphenyl-1H-pyrrol-3-yl)carbonyl]piperazin-2-yl}methyl)phenoxy]acetate

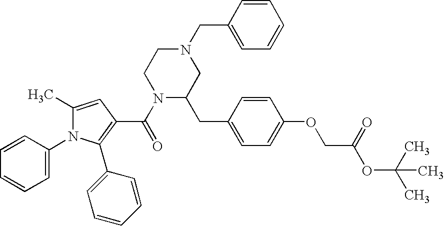

To a solution of 4-({4-benzyl-1-[(5-methyl-1,2-diphenyl-1H-pyrrol-3-yl)carbonyl]piperazin-2-yl}methyl)phenol (500 mg), tert-butyl bromoacetate (216 mg) and DMF (5 ml), was added potassium carbonate (191 mg). The mixture was stirred at 80° C. for 3 hr, then poured into ice-water, and the resulting suspension was filtered. The crystals were washed with hexane-ethyl acetate (1:1) and dried in vacuo to give the desired product (510 mg) as an amorphous solid.

MS (ESI+, m/e) 656 (M+1).

### Reference Example 242

### [4-({4-Benzyl-1-[(5-methyl-1,2-diphenyl-1H-pyrrol-3-yl)carbonyl]piperazin-2-yl}methyl)phenoxy]acetic acid hydrochloride

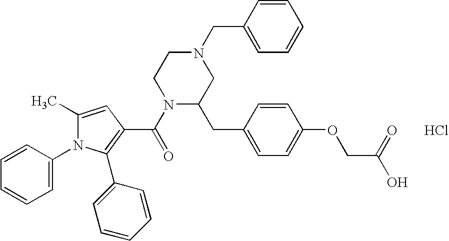

tert-Butyl [4-({4-benzyl-1-[(5-methyl-1,2-diphenyl-1H-pyrrol-3-yl)carbonyl]piperazin-2-yl}methyl)phenoxy]acetate (500 mg) was mixed with a 4 N hydrogen chloride-ethyl acetate solution (5 ml). The mixture was stirred at room temperature for 2 hr, and then the solvent was evaporated in vacuo. The residue was suspended in ethyl acetate, and the solvent was further evaporated in vacuo to give the desired product (500 mg) as an amorphous solid.

MS (ESI+, m/e) 600 (M+1)

### Reference Example 243

### 2-(4-({4-Benzyl-1-[(5-methyl-1,2-diphenyl-1H-pyrrol-3-yl)carbonyl]piperazin-2-yl}methyl)phenoxy]acetamide

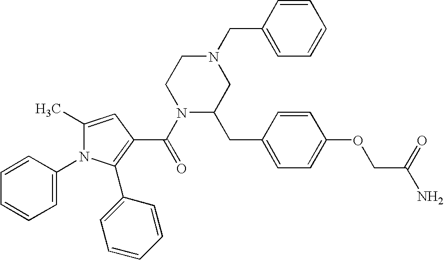

A solution of (4-({4-benzyl-1-[(5-methyl-1,2-diphenyl-1H-pyrrol-3-yl)carbonyl]piperazin-2-yl}methyl)phenoxy]acetic acid (150 mg), ammonium salts of HOBt (65 mg), WSC.HCl (72 mg) and DMF (3 ml) was stirred at room temperature for 12 hr. Then, the mixture was poured into a saturated aqueous sodium bicarbonate solution and extracted with ethyl acetate. The extract was washed successively with water and brine and dried over anhydrous sodium sulfate, and then the solvent was evaporated in vacuo. The residue was subjected to basic silica gel column chromatography, and the fraction eluted with ethyl acetate-methanol (4:1) was concentrated in vacuo to give the desired product (90 mg) as an amorphous solid.

MS (ESI+, m/e) 599 (M+1)

### Reference Example 244

### N-{[4-({4-Benzyl-1-[(5-methyl-1,2-diphenyl-1H-pyrrol-3-yl)carbonyl]piperazin-2-yl}methyl)phenoxy)acetyl}-β-alanine amide

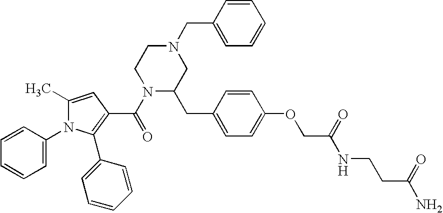

A solution of 4-({4-benzyl-1-[(5-methyl-1,2-diphenyl-1H-pyrrol-3-yl)carbonyl]piperazin-2-yl}methyl)phenol (150 mg), β-alanine amide (35 mg), WSC.HCl (66 mg), HOBt (46 mg) and DMF (3 ml) was stirred at room temperature for 12 hr. Then, the mixture was poured into a saturated aqueous sodium bicarbonate solution and extracted with ethyl acetate. The extract was washed with brine and dried over anhydrous sodium sulfate, and then the solvent was evaporated in vacuo. The residue was subjected to silica gel column chromatography, and the fraction eluted with ethyl acetate-methanol (4:1) was concentrated in vacuo to give the desired product (60 mg) as an amorphous solid.

MS (ESI+, m/e) 670 (M+l)

### Reference Example 245

### tert-Butyl (3-{[(2R)-2,4-dibenzylpiperazin-1-yl]carbonyl}-2-phenyl-1H-pyrrol-1-yl)acetate

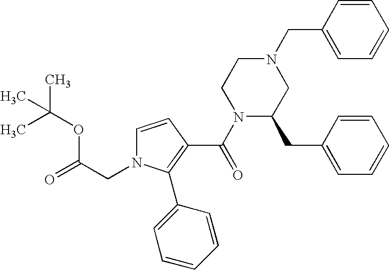

A solution of (2R)-2,4-dibenzyl-1-[(2-phenyl-1H-pyrrol-3-yl)carbonyl]piperazine (380 mg), tert-butyl bromoacetate (205 mg) and DMF (4 ml) was ice-cooled, and sodium hydride (60% in oil) (42 mg) was added thereto. After stirring at 0° C. for 15 min and at room temperature for 1 hr, the mixture was poured into an ice-cooled saturated aqueous sodium bicarbonate solution and extracted with ethyl acetate. The extract was dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (540 mg) as an amorphous solid.

MS (ESI+, m/e) 550 (M+1)

### Reference Example 246

### (3-{[(2R)-2,4-Dibenzylpiperazin-1-yl]carbonyl}-2-phenyl-1H-pyrrol-1-yl)acetic acid hydrochloride

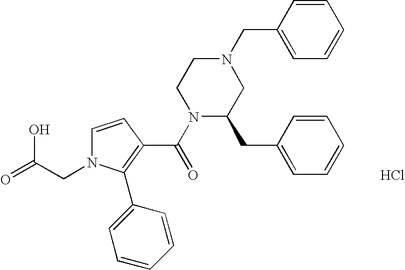

tert-Butyl (3-{[(2R)-2,4-dibenzylpiperazin-1-yl]carbonyl}-2-phenyl-1H-pyrrol-1-yl)acetate (480 mg) was mixed with a 4 N hydrogen chloride-ethyl acetate solution (5 ml), and the mixture was stirred at 40° C. for 5 hr. Then, the solvent was evaporated in vacuo to give the desired product (380 mg) as an amorphous solid.

MS (ESI+, m/e) 494 (M+1)

### Reference Example 247

### 2-(3-{[(2R)-2,4-Dibenzylpiperazin-1-yl]carbonyl}-2-phenyl-1H-pyrrol-1-yl)-N-(4-hydroxybutyl)acetamide

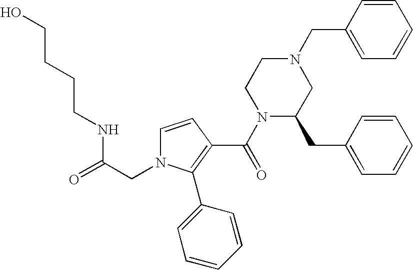

A solution of (3-{[(2R)-2,4-dibenzylpiperazin-1-yl]carbonyl}-2-phenyl-1H-pyrrol-1-yl)acetic acid hydrochloride (160 mg), 4-amino-1-butanol (40 mg), WSC.HCl (4.24 g), HOBt (2.74 g) and DMF (90 ml) was stirred at room temperature for 12 hr. Then, the mixture was poured into a saturated aqueous sodium bicarbonate solution and extracted with ethyl acetate. The extract was washed with brine and dried over anhydrous magnesium sulfate, and then the solvent was evaporated in vacuo. The residue was subjected to silica gel column chromatography, and the fraction eluted with ethyl acetate-hexane (1:1) was concentrated in vacuo to give the desired product (140 mg) as an amorphous solid.

MS (ESI+, m/e) 565 (M+1)

In the same manner as in Reference Example 247, the following compound (Reference Example 248) was obtained.

### Reference Example 248

### N-[(3-{[(2R)-2,4-Dibenzylpiperazin-1-yl]carbonyl}-2-phenyl-1H-pyrrol-1-yl)acetyl]-β-alanine amide

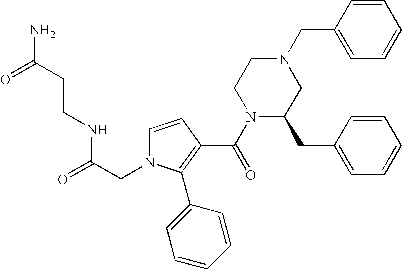

MS (ESI+, m/e) 564 (M+1)

### Reference Example 249

### (2R)-4-Benzyl-1-[(5-methyl-1,2-diphenyl-1H-pyrrol-3-yl)carbonyl]piperazine-2-carbaldehyde

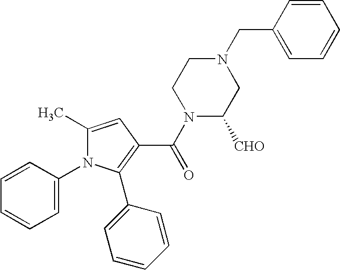

{(2R)-4-Benzyl-1-[(5-methyl-1,2-diphenyl-1H-pyrrol-3-yl)carbonyl]piperazin-2-yl}methanol (744 mg) was dissolved in dichloromethane (8 ml). A solution of pyridine sulfur trioxide complex (763 mg) in DMSO (8 ml), and triethylamine (0.67 ml) were added thereto at 0° C. After the reaction mixture was stirred at 0° C. for 2.5 hr, the mixture was poured into an ice-cooled saturated aqueous sodium bicarbonate solution and extracted with ethyl acetate. The extract was washed successively with water and brine and dried over anhydrous magnesium sulfate, and then the solvent was evaporated in vacuo. The residue was subjected to silica gel column chromatography, the fraction eluted with ethyl acetate-hexane (1:2 to 1:1) was concentrated in vacuo, and the crystals were collected by filtration to give the desired product (531 mg).

MS (ESI+, m/e) 464 (M+1)

In the same manner as in Reference Example 249, the following compound (Reference Example 250) was obtained.

### Reference Example 250

### (2S)-4-Benzyl-1-[(5-methyl-1,2-diphenyl-1H-pyrrol-3-yl)carbonyl]piperazine-2-carbaldehyde

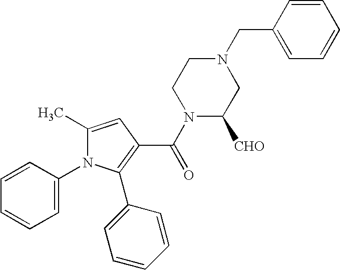

MS (ESI+, m/e) 464 (M+1)

### Reference Example 251

### tert-Butyl (3S)-3-(hydroxymethyl)-4-[(5-methyl-1,2-diphenyl-1H-pyrrol-3-yl)carbonyl]piperazine-1-carboxylate

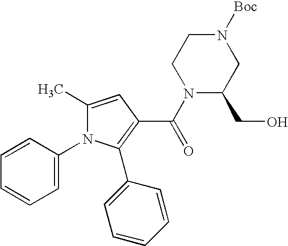

{(2S)-1-[(5-Methyl-1,2-diphenyl-1H-pyrrol-3-yl)carbonyl]piperazin-2-yl}methanol (the compound of Example 89 described hereinafter, 10.95 g) was dissolved in dioxane (150 ml). A 1 N aqueous sodium hydroxide solution (29.2 ml) and di-tert-butyl dicarbonate (7.00 g) were added thereto at 0° C. After stirring at room temperature for 2 hr, the mixture 20 was poured into water and extracted with ethyl acetate. The extract was washed successively with water and brine and dried over anhydrous magnesium sulfate, and then the solvent was evaporated in vacuo. The residue was subjected to silica gel column chromatography, the fraction eluted with ethyl acetate-hexane (1:2 to 1:0) was concentrated in vacuo, and the crystals were collected by filtration to give the desired product (12.70 g).

¹H-NMR (CDCl₃) δ 1.40 (9H, s), 2.10 (3H, s), 2.75-4.74 (9H, m), 6.21 (1H, br s), 7.03-7.30 (11H, m)

### Reference Example 252

### tert-Butyl (3S)-3-formyl-4-[(5-methyl-1,2-diphenyl-1H-pyrrol-3-yl)carbonyl]piperazine-1-carboxylate

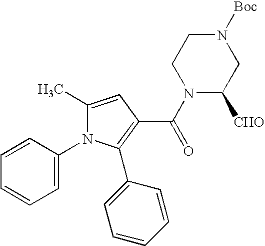

tert-Butyl (3S)-3-(hydroxymethyl)-4-[(5-methyl-1,2-diphenyl-1H-pyrrol-3-yl)carbonyl]piperazine-1-carboxylate (12.70 g) was dissolved in dichloromethane (140 ml). A solution of pyridine.sulfur trioxide complex (2.75 g) in DMSO (140 ml) and triethylamine (11.17 ml) were added thereto at 0° C. The reaction mixture was stirred at 0° C. for 2 hr, and then the mixture was poured into ice-water and extracted with ethyl acetate. The extract was washed successively with a 10% aqueous citric acid solution, water, a saturated aqueous sodium bicarbonate solution, water and brine and dried over anhydrous magnesium sulfate, and then the solvent was evaporated in vacuo. The residue was subjected to silica gel column chromatography, the fraction eluted with ethyl acetate-hexane (1:2 to 1:1) was concentrated in vacuo, and the crystals were collected by filtration to give the desired product (8.20 g).

MS (ESI+, m/e) 474 (M+1)

### Reference Example 253

### Methyl 1-(3-bromophenyl)-5-cyclohexyl-1H-pyrazole-4-carboxylate

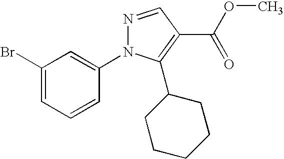

A solution of methyl 3-cyclohexyl-3-oxopropionate (3.00 g) and N,N-dimethylformamide dimethylacetal (3.00 g) and toluene (50 ml) was heated under reflux for 4 hr, and the reaction mixture was concentrated in vacuo. 3-Bromophenylhydrazine (2.83 g) and ethanol (50 ml) were added to the residue, and the mixture was heated under reflux for 15 hr. The reaction mixture was concentrated in vacuo, and the crystals were collected by filtration to give the desired product (4.60 g).

¹H-NMR (CDCl₃) δ 1.38 (3H, t), 1.60-1.71 (4H, m), 1.73-1.88 (4H, m), 2.09-2.23 (2H, m), 2.82-2.93 (1H, m), 4.11 (2H, q), 7.27-7.41 (2H, m), 7.55-7.65 (2H, m), 8.03 (1H, s)

### Reference Example 254

### 5-Cyclohexyl-1-(3-morpholinophenyl)-1H-pyrazole-4-carboxylic acid

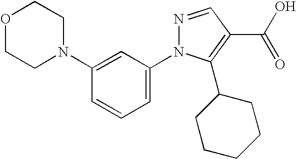

Methyl 1-(3-bromophenyl)-5-cyclohexyl-1H-pyrazole-4-carboxylate (2.30 g), morpholine (0.54 g), BINAP (0.38 g) and sodium tert-butoxide (0.60 g) were suspended in toluene (20 ml), Pd₂(dba)₃ (0.17 g) was added under argon atmosphere, and the mixture was stirred at 110° C. for 12 hr. Thereto were added ethyl acetate and water, and the mixture was extracted with ethyl acetate. The organic layer was dried over anhydrous magnesium sulfate, and concentrated in vacuo. The residue was purified by silica gel column chromatography to give methyl 5-cyclohexyl-1-(3-morpholinophenyl)-1H-pyrazole-4-carboxylate (1.90 g). This was dissolved in methanol (10 ml), and a 4 N aqueous lithium hydroxide solution (10 ml) was added. After heating under reflux for 12 hr, the reaction mixture was concentrated in vacuo. 2 N Hydrochloric acid was added to the residual aqueous solution to weakly acidify (pH 3) the mixture. This was extracted with ethyl acetate, the extract was washed with brine; and dried over anhydrous magnesium sulfate. The solvent was evaporated in vacuo. The residue was vacuum dried to give the desired product (1.90 g).

¹H-NMR (DMSO-d₆) δ 1.17 (1H, t), 1.26-1.35 (9H, m), 2.34-2.45 (2H, m), 2.46-2.53 (2H, m), 2.84 (1H, dd), 3.30-3.34 (2H, m), 3.46-3.55 (1H, m), 3.55-3.69 (1H, m), 3.84 (1H, s), 7.07-7.17 (1H, m), 7.19-7.26 (1H, m), 7.28-7.37 (3H, m)

### Reference Example 255

### 5-Cyclohexyl-1-(3-methoxyphenyl)-1H-pyrazole-4-carboxylic acid

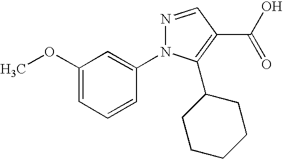

A solution of methyl 3-cyclohexyl-3-oxopropionate (2.20 g), N,N-dimethylformamide dimethylacetal (2.20 g) and toluene (20 ml) was heated under reflux for 4 hr, and the reaction mixture was concentrated in vacuo. 3-Methoxyphenylhydrazine (2.00 g) and ethanol (20 ml) were added to the residue, and the mixture was heated under reflux for 15 hr. The reaction mixture was concentrated in vacuo, and the crystals were collected by filtration to give methyl 5-cyclohexyl-1-(3-methoxyphenyl)-1H-pyrazole-4-carboxylate (1.80 g). This was dissolved in ethanol (7 ml), and a 4 N aqueous lithium hydroxide solution (13.7 ml) was added. After heating under reflux for 12 hr, the reaction mixture was concentrated in vacuo, 2 N hydrochloric acid was added to the residual aqueous solution to weakly acidify (pH 3) the mixture. This was extracted with ethyl acetate, and the extract was washed with brine, and dried over anhydrous magnesium sulfate. The solvent was evaporated in vacuo. The residue was vacuum dried to give the desired product (1.50 g).

¹H-NMR (DMSO-d₆) δ 1.04-1.18 (3H, m), 1.60 (3H, d), 1.65-1.79 (2H, m), 2.10 (2H, d), 2.82 (1H, t), 3.81 (3H, s), 6.95-6.99 (2H, m), 7.10-7.15 (1H, m), 7.45-7.51 (1H, m), 7.93 (1H, s), 12.34 (1H, br s)

### Reference Example 256

### 1-(2,3-Dihydro-1H-inden-2-yl)-5-phenyl-1H-pyrazole-4-carboxylic acid

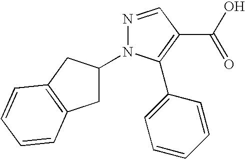

A solution of methyl 3-oxo-3-phenylpropionate (1.00 g), N,N-dimethylformamide dimethylacetal (1.00 g) and toluene (30 ml) was 4 hr heated under reflux, and the reaction mixture was is concentrated in vacuo. 2,3-Dihydro-1H-inden-2-ylhydrazine (0.77 g) and ethanol (30 ml) were added to the residue, and the mixture was heated under reflux for 15 hr. The reaction mixture was concentrated in vacuo, and the crystals were collected by filtration to give methyl 1-(2,3-dihydro-1H-inden-2-yl)-5-phenyl-1H-pyrazole-4-carboxylate (0.80 g). This was dissolved in methanol (7 ml) and a 4 N aqueous lithium hydroxide solution (7 ml) was added. After heating under reflux for 12 hr, the reaction mixture was concentrated in vacuo, and 2 N hydrochloric acid was added to the residual aqueous solution to weakly acidify (pH 3) the mixture. This was extracted with ethyl acetate, and the extract was washed with brine, and dried over anhydrous magnesium sulfate. The solvent was evaporated in vacuo and the residue was vacuum dried to give the desired product (0.61 g).

¹H-NMR (DMSO-d₆) δ 3.20-3.29 (3H, m), 3.38 (1H, d), 4.83-4.94 (1H, m), 7.12-7.21 (4H, m), 7.44-7.54 (5H, m), 7.92 (1H, s), 11.73-12.37 (1H, m)

### Reference Example 257

### 1-(2,3-Dihydro-1H-inden-2-yl)-2-phenylethanone

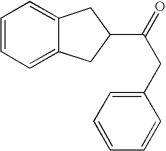

N-Methoxy-N-methylindane-2-carboxamide (2.50 g) was dissolved in THF (50 ml), and the mixture was cooled to 0° C. Benzylmagnesium bromide (1 M THF solution, 18.3 ml) was added thereto and the mixture was stirred at the same temperature for 2 hr, and at room temperature for 5 hr. A saturated aqueous ammonium chloride solution was added to the reaction mixture, and the mixture was extracted with ethyl acetate. The extract was washed with brine, dried over anhydrous magnesium sulfate, and concentrated in vacuo. The residue was subjected to silica gel column chromatography, and the fraction eluted with ethyl acetate was concentrated in vacuo to give the desired product (1.52 g) as an oil.

NMR (CDCl₃) δ 3.03-3.26 (4H, m), 3.44-3.62 (1H, m), 3.82 (2H, s), 7.12-7.37 (5H, m), 7.12-7.37 (4H, m)

### Reference Example 258

### 1-(3-Morpholinophenyl)-2-phenylethanone

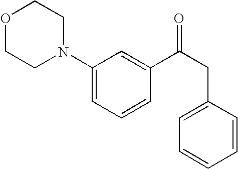

1-(3-Bromophenyl)-2-phenylethanone (4.2 g), BINAP (285 mg), sodium tert-butoxide (2.2 g), Pd₂(dba)3 (137 mg) and morpholine (1.85 ml) were mixed with toluene (5 ml) under argon atmosphere. After stirring at 100° C. for 8 hr, and the mixture was diluted with ethyl acetate. The organic layer was washed with water, and dried over anhydrous sodium sulfate. The solvent was evaporated in vacuo. The residue was subjected to silica gel column chromatography, and the fraction eluted with ethyl acetate-hexane (6:4) was concentrated in vacuo to give the desired product (1.63 g) as an oil.

NMR (CDCl₃) δ 3.04-3.21 (4H, m), 3.66-3.93 (4H, m), 4.16-4.33 (2H, m), 6.76-7.78 (9H, m)

### Reference Example 259

### Ethyl 4-(2,3-dihydro-1H-inden-2-yl)-2,4-dioxo-3-phenylbutanoate

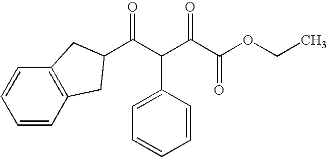

1-(2,3-Dihydro-1H-inden-2-yl)-2-phenylethanone (1.52 g) and diethyl oxalate (3.76 g) were dissolved in ethanol (50 ml), and a solution of sodium ethoxide (1.75 g) in ethanol (50 ml) was added. After heating the reaction mixture under reflux for 1 hr, the mixture was poured into 1 N hydrochloric acid, and the mixture was extracted with ethyl acetate. The extract was washed successively with saturated aqueous sodium bicarbonate solution and brine, dried over anhydrous magnesium sulfate, and concentrated in vacuo. The residue was subjected to silica gel column chromatography, and the fraction eluted with ethyl acetate-hexane (1:19 to 1:1) was concentrated in vacuo to give the desired product (1.47 g) as an oil.

NMR (CDCl₃) δ 0.96 (3H, t); 2.84-2.97 (2H, m), 3.21-3.48 (3H, m), 4.00 (2H, q), 7.08-7.43. (9H, m), 15.84 (1H, s)

In the same manner as in Reference Example 259, the following compound (Reference Example 260) was obtained.

### Reference Example 260

### Ethyl 4-(3-morpholinophenyl)-2,4-dioxo-3-phenylbutanoate

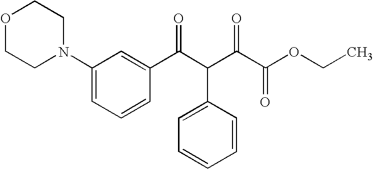

NMR (CDCl₃) δ 0.91-1.39 (3H, m), 2.77-3.31 (4H, m), 3.67-3.95 (4H, m), 3.93-4.40 (2H, m), 6.33 (1H, s), 6.67-7.62 (9H, m)

### Reference Example 261

### Ethyl 3,4-diphenyl-1H-pyrazole-5-carboxylate

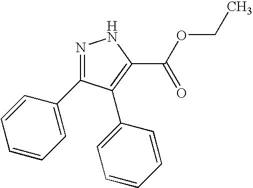

Ethyl 2,4-dioxo-3,4-diphenylbutanoate (2.5 g) was dissolved in ethanol (30 ml), hydrazine monohydrate (0.42 g) was added, and the mixture was heated under reflux for 15 hr. The reaction mixture was concentrated in vacuo, the residue was subjected to silica gel column chromatography, and the target fraction was concentrated in vacuo to give the desired product (0.51 g).

¹H-NMR, (CDCl₃) δ 1.17-1.26 (3H, m), 4.26 (2H, q), 7.24-7.39 (10H, m)

In the same manner as in Reference Example 261, the following compounds (Reference Examples 262 and 263) were obtained.

### Reference Example 262

### Ethyl 3-(2,3-dihydro-1H-inden-2-yl)-4-phenyl-1H-pyrazole-5-carboxylate

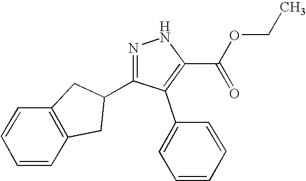

¹H-NMR (CDCl₃) δ 1.22-1.30 (3H, m), 3.17 (4H, d), 3.66-3.77 (1H, m), 4.24 (2H, q), 7.13-7.21 (4H, m), 7.32-7.43 (5H, m), 10.51 (1H, br s)

### Reference Example 263

### Ethyl 3-(3-morpholinophenyl)-4-phenyl-1H-pyrazole-5-carboxylate

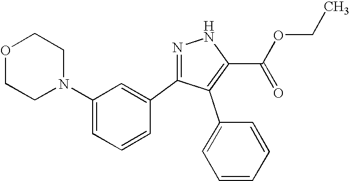

¹H-NMR (DMSO-d₆) δ 1.08 (3H, t), 2.83-2.98 (4H, m), 3.57-3.70 (4H, m), 4.12 (2H, q), 6.71-6.91 (3H, m), 7.10-7.39 (7H, m)

### Reference Example 264

### Ethyl 1-benzyl-3,4-diphenyl-1H-pyrazole-5-carboxylate

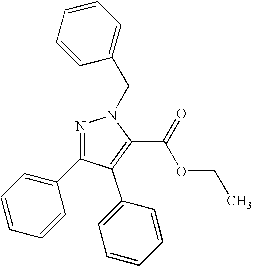

To a solution of ethyl 3,4-diphenyl-1H-pyrazole-5-carboxylate (473 mg), benzyl bromide (332 mg) and DMA (10 ml) was added cesium carbonate (1.06 g). After stirring at 60° C. for 17 hr, the reaction mixture was poured into water, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (4:6) was concentrated in vacuo to give the desired product (523 mg) as an amorphous solid.

¹H-NMR (CDCl₃) δ 0.92 (3H, t), 4.05 (2H, q), 5.83 (2H, s), 7.14-7.47 (15H, m)

In the same manner as in Reference Example 264, the following compound (Reference Example 265) was obtained.

### Reference Example 265

### Ethyl 3,4-diphenyl-1-[3-(1H-pyrrol-1-yl)benzyl]-1H-pyrazole-5-carboxylate

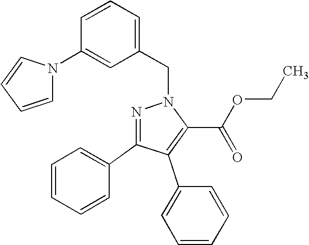

¹H-NMR (DMSO-d₆) δ 0.86 (3H, t), 4.04 (2H, q), 5.82 (2H, s), 6.26-6.33 (2H, m), 7.00-7.15 (1H, m), 7.22-7.38 (12H, m), 7.42-7.55 (3H, m)

### Reference Example 266

### 3,4-Diphenyl-1H-pyrazole-5-carboxylic acid

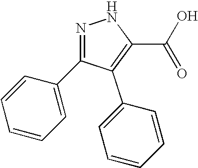

Ethyl 3,4-diphenyl-1H-pyrazole-5-carboxylate (0.51 g) was dissolved in methanol-THF (1:1, 10 ml), and a 4 N aqueous lithium hydroxide solution (4.3 ml) was added. After heating under reflux for 12 hr, the reaction mixture was concentrated in vacuo, 2 N hydrochloric acid was added to the residual aqueous solution to weakly acidify (pH 3) the mixture. This was extracted with ethyl acetate, and the extract was washed with brine, and dried over anhydrous magnesium sulfate. The solvent was evaporated in vacuo and the residue was vacuum dried to give the desired product (0.45 g).

¹H-NMR (DMSO-d₆) δ 7.20-7.34 (10H, m), 13.09-14.51 (1H, m)

In the same manner as in Reference Example 266, the following compounds (Reference Examples 267 to 269) were obtained.

### Reference Example 267

### 3-(2,3-Dihydro-1H-inden-2-yl)-4-phenyl-1H-pyrazole-5-carboxylic acid

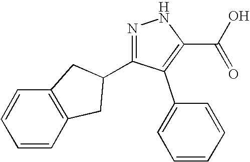

NMR (DMSO-d₆) δ 2.94-3.25 (4H, m), 3.47-3.63 (1H, m), 4.09 (1H, d), 7.08-7.20 (4H, m), 7.27-7.41 (5H, m), 13.22 (1H, br s)

### Reference Example 268

### 3-(3-Morpholinophenyl)-4-phenyl-1H-pyrazole-5-carboxylic acid

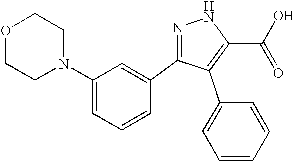

NMR (DMSO-d₆) δ 2.85-2.95 (4H, m), 3.59-3.69 (4H, m), 6.76-6.91 (3H, m), 7.14 (1H, t), 7.19-7.26 (2H, m), 7.26-7.38 (3H, m)

### Reference Example 269

### 3,4-Diphenylpyridine-2-carboxylic acid

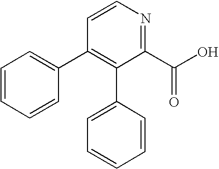

MS (ESI+, m/e) 276 (M+1)

### Reference Example 270

### 1-Benzyl-3,4-diphenyl-1H-pyrazole-5-carboxylic acid

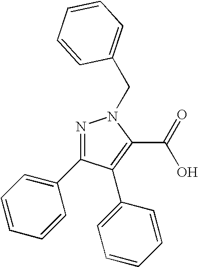

Ethyl 1-benzyl-3,4-diphenyl-1H-pyrazole-5-carboxylate (510 mg) was suspended in ethanol (20 ml), and a 4 N aqueous sodium hydroxide solution (3.3 ml) was added. After heating under reflux for 1 hr, the mixture was weakly acidified (pH 3) with 1 N hydrochloric acid and extracted with ethyl acetate. The extract was washed with brine, and dried over anhydrous sodium sulfate, and the solvent was evaporated in vacuo to give the desired product (467 mg) as an amorphous solid.

NMR (CDCl₃) δ 5.85 (2H, s), 7.14-7.42 (15H, m)

In the same manner as in Reference Example 270, the following compound (Reference Example 271) was obtained.

### Reference Example 271

### 3,4-Diphenyl-1-[3-(1H-pyrrol-1-yl)benzyl]-1H-pyrazole-5-carboxylic acid

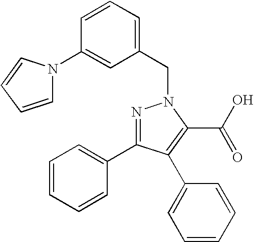

NMR (DMSO-d₆) δ 5.84 (2H, s), 6.19-6.36 (2H, m), 7.03-7.11 (1H, m), 7.19-7.55 (15H, m), 13.34 (1H, brs)

### Reference Example 272

### Methyl 4-(3-bromophenyl)-3-phenyl-1H-pyrrole-2-carboxylate

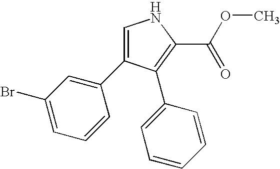

(2Z)-2-(3-Bromophenyl)-3-phenylacrylonitrile (3.0 g) and methyl isocyanoacetate (1.4 g) were dissolved in THF (45 ml). The mixture was ice-cooled, potassium tert-butoxide (2.3 g) was added and the mixture was stirred at room temperature for 15 hr. An aqueous ammonium chloride solution was added to the reaction mixture, and the mixture was extracted with ethyl acetate. The extract was washed with brine, and dried over anhydrous magnesium sulfate. The solvent was evaporated in vacuo. The residue was subjected to silica gel column chromatography, and the target fraction was concentrated in vacuo to give the desired product (1.40 g).

¹H-NMR (DMSO-d₆) δ 3.61 (3H, s), 7.00-7.05 (1H, m), 7.07-7.21 (4H, m), 7.25-7.40 (5H, m), 12.20 (1H, s)

### Reference Example 273

### Methyl 4-(3-bromophenyl)-5-formyl-3-phenyl-1H-pyrrole-2-carboxylate

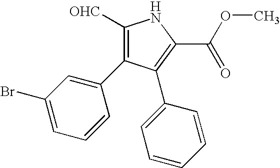

DMF (0.78 ml) was dissolved in dichloroethane (2.25 ml), the mixture was ice-cooled, and phosphoryl chloride (0.95 ml) was added dropwise. After stirring at room temperature for 1 hr, the reaction mixture was ice-cooled again. A solution of methyl 4-(3-bromophenyl)-3-phenyl-1H-pyrrole-2-carboxylate (3.2 g) in dichloroethane (2.25 ml) was added dropwise, and the mixture was stirred at room temperature for 2 hr. The reaction mixture was poured into water, and extracted with ethyl acetate. The extract was washed with brine, and dried over anhydrous magnesium sulfate. The solvent was evaporated in vacuo. The residue was subjected to silica gel column chromatography, and the target fraction was concentrated in vacuo to give the desired product (2.0 g).

¹H-NMR (CDCl₃) δ 3.79 (3H, s), 7.01-7.07 (1H, m), 7.10-7.20 (3H, m), 7.26-7.35 (4H, m), 7.42 (1H, d), 9.63 (1H, s), 9.98 (1H, s)

### Reference Example 274

### 4-(3-Bromophenyl)-3-phenyl-1H-pyrrole-2-carboxylic acid

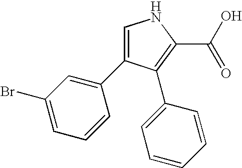

Methyl 4-(3-bromophenyl)-3-phenyl-1H-pyrrole-2-carboxylate (0.27 g) was dissolved in methanol-THF (1:1, 2.0 ml), and a 2 N aqueous sodium hydroxide solution (1.0 ml) was added. After heating under reflux for 12 hr, the reaction mixture was concentrated in vacuo, and 2 N hydrochloric acid was added to the residual aqueous solution to weakly acidify (pH 3) the mixture. This was extracted with ethyl acetate, and the extract was washed with brine, and dried over anhydrous magnesium sulfate. The solvent was evaporated in vacuo and the residue was vacuum dried to give the desired product (0.24 g).

¹H-NMR (CDCl₃) δ 6.89-7.04 (2H; m), 7.10 (1H, d), 7.22-7.36 (8H, m), 9.31 (1H, s)

In the same manner as in Reference Example 274, the following compound (Reference Example 275) was obtained.

### Reference Example 275

### 4-(3-Bromophenyl)-5-formyl-3-phenyl-1H-pyrrole-2-carboxylic acid

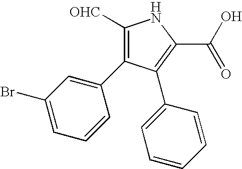

MS (ESI+, m/e) 370 (M+1)

### Reference Example 276

### 4-(3-Bromophenyl)-3-phenyl-1-(1,3-thiazol-4-ylmethyl)-1H-pyrrole-2-carboxylic acid

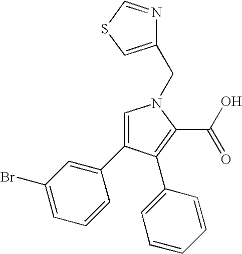

A solution of methyl 4-(3-bramophenyl)-3-phenyl-1H-pyrrole-2-carboxylate (1.0 g), 1,3-thiazol-4-ylmethyl chloride (0.57 g) and cesium carbonate (1.3 g) in DMA (10 ml) was stirred at 80° C. for 5 hr. The reaction mixture was poured into water and extracted with ethyl acetate. The extract was washed with brine, and dried over anhydrous magnesium sulfate. The solvent was evaporated in vacuo. The residue was subjected to silica gel column chromatography, and the target fraction was concentrated in vacuo. The residue was dissolved in methanol-THF (1:1, 10 ml), and a 4 N aqueous lithium hydroxide solution (5 ml) was added. After heating under reflux for 12 hr, the reaction mixture was concentrated in vacuo, and 2 N hydrochloric acid was added to the residual aqueous solution to weakly acidify (pH 3) the mixture. This was extracted with ethyl acetate, and the extract was washed with brine, and dried over anhydrous magnesium sulfate. The solvent was evaporated in vacuo and the residue was vacuum dried to give the desired product (0.32 g).

¹H-NMR (CDCl₃) δ 5.76 (2H, s), 6.85-6.89 (1H, m), 6.97 (1H, t), 7.16-7.31 (9H, m), 8.81 (1H, d)

### Reference Example 277

### Ethyl 2-(formylamino)-3-phenylacrylate

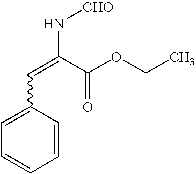

Sodium hydride (60% in oil) (11.62 g) was suspended in THF (270 ml), and a solution of benzaldehyde (28.27 g) and ethyl isocyanoacetate (27.39 g) in THF (55 ml) was added dropwise with stirring at room temperature over 20 min. After stirring at room temperature for 2.5 hr, the reaction mixture was ice-cooled, and acetic acid (45 ml) was added dropwise. After stirring for 10 min, the mixture was poured into ice-water and extracted with ethyl acetate. The extract was washed successively with water, saturated aqueous sodium bicarbonate solution, water and brine, and dried over anhydrous magnesium sulfate. The solvent was evaporated in vacuo. The residue was subjected to silica gel column chromatography, and the fraction eluted with ethyl acetate-hexane (1:2 to 2:1) was concentrated in vacuo to give the desired product (40.27 g) as an oil.

¹H-NMR (CDCl₃) δ 0.98-1.40 (3H, m), 4.06-4.38 (2H, m), 7.06-7.68 (7H, m), 8.21-8.47 (1H, m)

### Reference Example 278

### Ethyl 3-bromo-2-(formylamino)-3-phenylacrylate

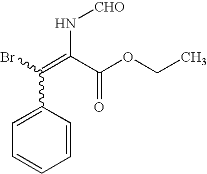

Ethyl 2-(formylamino)-3-phenylacrylate (40.27 g) was dissolved in carbon tetrachloride-chloroform (3:1, 440 ml). The mixture was ice-cooled, and NBS (34.33g) was added. After stirring at 0° C. for 1.5 hr and at room temperature for 3 hr, the reaction mixture was ice-cooled again, and triethylamine (19.52 g) was added. After stirring at 0° C. for 20 min and at room temperature for 40 min, the reaction mixture was washed successively with water and brine, and dried over anhydrous magnesium sulfate. The solvent was evaporated in vacuo. The residue was subjected to silica gel column chromatography, and the fraction eluted with ethyl acetate-hexane (1:3 to 1:2) was concentrated in vacuo to give the desired product (44.88 g) as an oil.

¹H-NMR (CDCl₃) δ 0.89-1.45 (3H, in), 3.97-4.46 (2H, m) , 6.91 (1H, br s), 7.28-7.46 (5H, m), 7.95-8.28 (1H, m)

### Reference Example 279

### Ethyl 3-bromo-2-isocyano-3-phenylacrylate

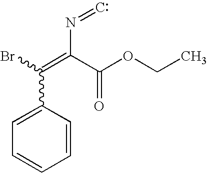

Ethyl 3-bromo-2-(formylamino)-3-phenylacrylate (16.33 g) and triethylamine (13.86 g) were dissolved in dichloromethane (150 ml) and the mixture was ice-cooled. Phosphoryl chloride (9.24 g) was added, and the mixture was vigorously stirred at 0° C. for 2 hr. The reaction mixture was poured into saturated aqueous sodium bicarbonate solution. After vigorously stirring at room temperature for 1 hr, the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:6) was concentrated in vacuo at not more than 30° C. to give the desired product (14.82 g) as an oil.

¹H-NMR (CDCl₃) δ 1.03-1.42 (3H, m), 4.04-4.42 (2H, m), 7.25-7.56 (5H, m)

### Reference Example 280

### 3-(3-Methoxypropoxy)benzaldehyde

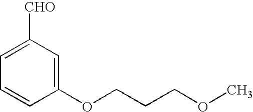

3-Hydroxybenzaldehyde (7.26 g) and 1-bromo-3-methoxypropane (9.10 g) were dissolved in DMF (60 ml), potassium carbonate (9.86 g) was added, and the mixture was stirred at 50° C. for 3 hr. The reaction mixture was poured into water, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:6) was concentrated in vacuo to give the desired product (9.30 g) as an amorphous solid.

¹H-NMR (CDCl₃) δ 2.07 (2H, quintet), 3.36 (3H, s), 3.56 (2H, t), 4.12 (2H, t), 7.15-7.19 (1H, m), 7.39-7.45 (3H, m), 9.96 (1H, s)

### Reference Example 281

### Methyl 2-(formylamino)-3-(3-(3-methoxypropoxy)phenyl)acrylate

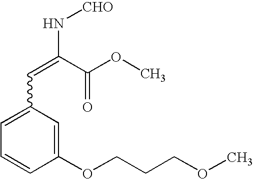

Sodium hydride (60% in oil) (2.30 g) was suspended in THF (55 ml), and a solution of 3-(3-methoxypropoxy)benzaldehyde (9.29 g) and methyl isocyanoacetate (4.74 g) in THF (10 ml) was added dropwise with stirring at room temperature over 5 min. After stirring at room temperature for 1 hr, the reaction mixture was ice-cooled, and acetic acid (9 ml) was added dropwise. After stirring for 10 min, the mixture was poured into ice-water and extracted with ethyl acetate. The extract was washed successively with water, saturated aqueous sodium bicarbonate solution, water and brine, and dried over anhydrous magnesium sulfate. The solvent was evaporated in vacuo. The residue was subjected to silica gel column chromatography, and the fraction eluted with ethyl acetate-hexane (1:1.5 to 2:1) was concentrated in vacuo to give the desired product (9.82 g) as an oil.

¹H-NMR (CDCl₃) δ 2.04 (2H, quintet), 3.35 (3H, s), 3.55 (2H, t), 3.66 (1H, s), 3.89 (2H, s), 4.05 (2H, t), 6.82-7.62 (5H, m), 8.16-8.40 (2H, m)

### Reference Example 282

### Methyl 3-bromo-2-(formylamino)-3-[3-(3-methoxypropoxy)phenyl]acrylate

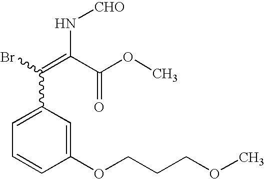

Methyl 2-(formylamino)-3-[3-(3-methoxypropoxy)phenyl]acrylate (9.81 g) was dissolved in carbon tetrachloride-chloroform (3:1, 80 ml). The mixture was ice-cooled, and NBS (6.25 g) was added. After stirring at 0° C. for 1.5 hr and at room temperature for 3 hr, the reaction mixture was ice-cooled again, and triethylamine (3.55 g) was added. After stirring at 0° C. for 20 min and at room temperature for 40 min, the reaction mixture was washed successively with water and brine, and dried over anhydrous magnesium sulfate. The solvent was evaporated in vacuo. The residue was subjected to silica gel column chromatography, and the fraction eluted with ethyl acetate-hexane (1:3 to 1:1) was concentrated in vacuo to give the desired product (11.20 g) as an oil.

¹H-NMR (CDCl₃) δ 2.00-2.09 (2H, M), 3.50 (3H, s), 3.52-3.58 (3H, m), 3.93-4.11 (4H, m), 6.77-6.98 (3H, m), 7.21-7.36 (2H, m), 7.96 (0.5H, s), 8.29 (0.5H, s)

### Reference Example 283

### tert-Butyl [(1S)-3-morpholino-3-oxo-1-(2-phenylethyl)propyl]carbamate

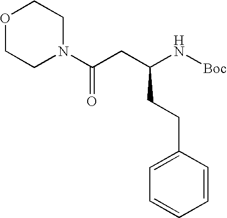

A solution of (3S)-3-[(tert-butoxycarbonyl)amino]-5-phenylvaleric acid (1.00 g), morpholine (328 mg), WSC.HCl (787 mg), HOBt (508 mg) and DMF (17 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo to give the desired product (1.23 g) as an oil.

¹H-NMR (CDCl₃) δ 1.44 (9H, s), 1.84-2.00 (2H, m), 2.47 (1H, dd), 2.57-2.75 (3H, m), 3.40-3.41.(2H, m), 3.49-3.67 (6H, m), 3.83-3.88 (1H, m), 5.25 (1H, br d), 7.14-7.19 (3H, m), 7.24-7.29 (2H, m)

MS (ESI+, m/e) 363 (M+1)

In the same manner as in Reference Example 283, the following compound (Reference Example 284) was obtained.

### Reference Example 284

### tert-Butyl [(1R)-3-morpholino-3-oxo-1-(2-phenylethyl)propyl]carbamate

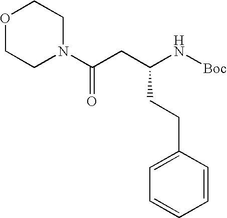

¹H-NMR (CDCl₃) δ 1.44 (9H, s), 1.83-2.03 (2H, m), 2.47 (1H, dd), 2.57-2.79 (3H, m), 3.40-3.41 (2H, m), 3.49-3.67 (6H, m), 3.84-3.88 (1H, m), 5.25 (1H, br d), 7.14-7.19 (3H, m), 7.24-7.29 (2H, m)

MS (ESI+, m/e) 363 (M+1)

### Reference Example 285

### (3S)-1-Morpholino-1-oxo-5-phenylpentan-3-amine

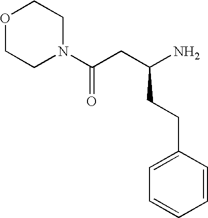

To a solution of tert-butyl [(1S)-3-morpholino-3-oxo-1-(2-phenylethyl)propyl)carbamate (1.22 g) in dichloromethane (0.9 ml) was added TFA (9 ml), and the mixture was stirred at room temperature for 1 hr. The reaction mixture was poured into saturated aqueous sodium bicarbonate solution by small portions, and the mixture was basified by adding potassium carbonate by small portions. The mixture was saturated with sodium chloride, and extracted with ethyl acetate. The extract was washed with brine, and dried over anhydrous magnesium sulfate. The solvent was evaporated in vacuo to give the desired product (847 mg) as an oil.

¹H-NMR (CDCl₃) δ 1.89-2.01 (1H, m), 2.10-2.22 (1H, m), 2.57 (1H, dd), 2.62-2.77 (3H, m), 3.2.7-3.37 (2H, m), 3.49-3.64 (7H, m), 5.72 (2H, br. s), 7.14-7.20 (3H, m), 7.24-7.29 (2H, m)

MS (ESI+, m/e) 263 (M+1)

In the same manner as in Reference Example 285, the following compound (Reference Example 286) was obtained.

### Reference Example 286

### (3R)-1-Morpholino-1-oxo-5-phenylpentan-3-amine

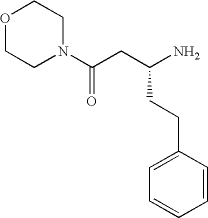

¹H-NMR (CDCl₃) δ 1.91-2.03 (1H, m), 2.11-2.20 (1H, m), 2.57 (1H, dd), 2.63-2.78 (3H, m), 3.27-3.37 (2H, m), 3.49-3.64 (7H, m), 5.22 (2H, br s), 7.14-7.21 (3H, m), 7.24-7.29 (2H, m)

MS (ESI+, m/e) 263 (M+1)

### Reference Example 287

### Benzyl ((1R)-1-{[(tert-Butoxycarbonyl)amino]methyl}-2-morpholino-2-7oxoethyl)carbamate

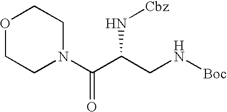

A solution of N-[(benzyloxy)carbonyl]-3-[(tert-butoxycarbonyl)amino]-D-alanine (4.99 g), morpholine (1.41 g), WSC.HCl (3.39 g), HOBt (2.19.g) and DMF (75 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo to give the desired product (6.01 g) as an amorphous solid.

¹H-NMR (CDCl₃) δ 1.43 (9H, s), 3.20-3.73 (10H, m), 4.75-4.81 (1H, m), 5.00 (1H, br t), 5.09 (2H, s), 5.87 (1H, br d), 7.29-7.35 (5H, m)

MS (ESI+, m/e) 308 (M+1-“Boc”)

In the same manner as in Reference Example 287, the following compounds (Reference Examples 288 and 289) were obtained.

### Reference Example 288

### tert-Butyl {(1R)-1-(2-(methylthio)ethyl]-3-morpholino-3-oxopropyl}carbamate

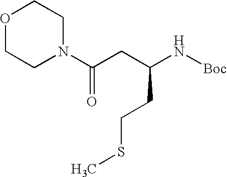

¹H-NMR (CDCl₃) δ 1.43 (9H, s), 1.79-1.88 (1H, m), 1.94-1.99 (1H, m), 2.10 (3H, s), 2.46-2.68 (4H, m), 3.47-3.49 (2H, m), 3.58-3.61 (2H, m), 3.65-3.68 (4H, m), 3.95-4.03 (1H, m), 5.31 (1H, br d)

MS (ESI+, m/e) 233 (M+1-“Boc”)

### Reference Example 289

### Benzyl (3R)-3-[(tert-butoxycarbonyl)amino]-5-morpholino-5-oxovalerate

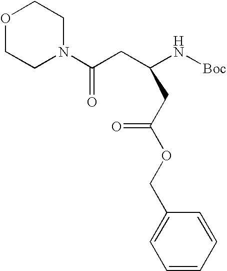

¹H-NMR (CDCl₃) δ 1.42 (9H, s), 2.58 (1H, dd), 2.66-2.76 (2H, m), 2.85 (1H, dd), 3.37-3.40 (2H, m), 3.56-3.65 (6H, m), 4.24-4.32 (1H, m), 5.09 (1H, dd), 5.14 (1H, dd), 5.61 (1H, br d), 7.29-7.38 (5H, m)

MS (ESI+, m/e) 307 (M+1-“Boc”)

### Reference Example 290

### (3R)-3-[(tert-Butoxycarbonyl)amino]-5-morpholino-5-oxovaleric acid

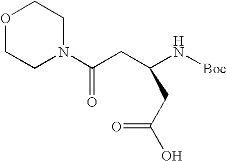

Benzyl (3R)-3-[(tert-butoxycarbonyl)amino]-5-morpholino-5-oxovalerate (6.02 g) was dissolved in THF-ethanol (1:1, 175 ml), 10% palladium on carbon (containing 50% water) (3.0 g) was added, and a catalytic hydrogenation was performed at room temperature and atmospheric pressure for 15 hr. The catalyst was filtered off, the filtrate was concentrated in vacuo, and the crystals were collected by filtration to give the desired product (4.30 g).

¹H-NMR (CDCl₃) δ 1.43 (9H, s), 2.65-2.86 (4H, m), 3.55-3.75 (9H, m), 4.17-4.21 (1H, m), 5.82 (1H, br d)

MS (ESI+, m/e) 217 (M+1-“Boc”)

### Reference Example 291

### tert-Butyl [(1S)-3-[(2-furylmethyl)amino]-1-(2-morpholino-2-oxoethyl)-3-oxopropyl]carbamate

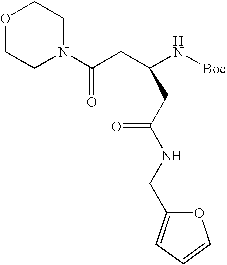

A solution of (3R)-3-[(tert-butoxycarbonyl)amino]-5-morpholino-5-oxovaleric acid (1.08 g), furfurylamine (363 mg), WSC.HCl (782 mg), HOBt (505 mg) and DMF (12 ml) wits stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo, and the crystals were collected by filtration to give the desired product (1.02 g).

¹H-NMR (CDCl₃) δ 1.42 (9H, s), 2.41-2.54 (2H, m), 2.63 (1H, dd), 2.89 (1H, dd), 3.51-3.71 (8H, m), 4.08-4.11 (1H, m), 4.43-4.44 (2H, m), 5.90 (1H, br d), 6.24 (1H, dd), 6.31 (1H, dd), 6.79 (1H, br s), 7.35 (1H, dd)

MS (ESI+, m/e) 396 (M+1)

### Reference Example 292

### Benzyl [(1R)-1-(aminomethyl)-2-morpholino-2-oxoethyl]carbamate

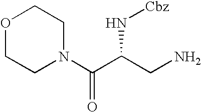

To a solution of benzyl ((1R)-1-{[(tert-butoxycarbonyl)amino]methyl}-2-morpholino-2-oxoethyl)carbamate (5.97 g) in dichloromethane (3 ml) was added TFA (30 ml), and the mixture was stirred at room temperature for 40 min. The reaction mixture was poured into saturated aqueous sodium bicarbonate solution by small portions, and the mixture was basified by adding potassium carbonate by small portions. The mixture was saturated with sodium chloride, and extracted with chloroform. The extract was washed with brine, and dried over anhydrous magnesium sulfate. The solvent was evaporated in vacuo to give the desired product (4.11 g) as an oil.

¹H-NMR (CDCl₃) δ 2.00 (2H, s), 2.89 (1H, dd), 3.03 (1H, dd), 3.58-3.66 (8H, m), 4.64-4.70 (1H, m), 5.08 (1H, s), 5.12 (1H, s), 5.99 (1H, d), 7.29-7.38 (5H, m)

MS (ESI+, m/e) 308 (M+1)

In the same manner as in Reference Example 292, the following compounds (Reference Examples 293 and 294) were obtained.

### Reference Example 293

### (3R)-5-(Methylthio)-1-morpholino-1-oxopentan-3-amine

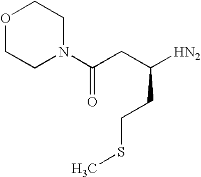

¹H-NMR (CDCl₃) δ 1.67-1.77 (2H, m), 1.84 (2H, s), 2.11 (3H, s), 2.30 (1H, dd), 2.46 (1H, dd), 2.52-2.65 (2H, m), 3.38-3.47 (3H, m), 3.60-3.69 (6H, m)

MS (ESI+, m/e) 233 (M+1)

### Reference Example 294

### (3S)-3-Amino-N-(2-furylmethyl)-5-morpholino-5-oxopentanamide

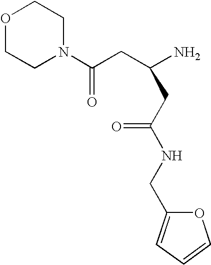

¹H-NMR (CDCl₃) δ 1.80 (2H, s), 2.28-2.54 (4H, m), 3.41-3.44 (2H, m), 3.58-3.68 (7H, m), 4.36-4.51 (2H, m), 6.22-6.23 (1H, m), 6.31-6.32 (1H, m), 7.34-7.35 (1H, m), 7.69 (1H, br s)

MS (ESI+, m/e) 296 (M+1)

### Reference Example 295

### Benzyl [2-(tetrahydro-2H-pyran-4-ylamino)ethyl]carbamate

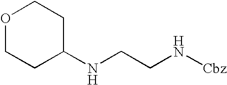

A solution of benzyl (2-aminoethyl)carbamate hydrochloride (10.44 g), tetrahydro-4H-pyran-4-one (5.44 g), acetic acid (5.44 g), triethylamine (5.04 g), dichloromethane (180 ml) and DMF (90 ml) was stirred at room temperature for 1 hr, sodium triacetoxyborohydride (19.18 g) was added by small portions over 5 min. The mixture was stirred at room temperature for additional 15 hr, and concentrated to about half in vacuo. The concentrate was poured into saturated aqueous sodium bicarbonate solution, and basified with potassium carbonate. The mixture was stirred at room temperature for 30 min and extracted with ethyl acetate. The extract was washed successively with water and brine, and dried over anhydrous magnesium-sulfate. The solvent was evaporated in vacuo. The residue was subjected to basic silica gel column chromatography, and the fraction eluted with ethyl acetate-hexane (1:1 to 1:0) was concentrated in vacuo to give the desired product (8.32 g) as an oil.

¹H-NMR (CDCl₃) δ 1.28-1.41 (3H, m), 1.80 (2H, d), 2.60-2.67 (1H, m), 2.77 (2H, t), 3.28 (2H, q), 3.37 (2H, dt), 3.95 (2H, dt), 5.10 (2H, s), 5.21 (1H, br s), 7.29-7.38 (5H, m)

MS (ESI+, m/e) 279 (M+1)

### Reference Example 296

### Benzyl {2-[(tert-butoxycarbonyl)(tetrahydro-2H-pyran-4-yl)amino]ethyl}carbamate

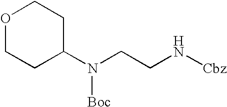

Benzyl [2-(tetrahydro-2H-pyran-4-ylamino)ethyl]carbamate (8.32 g) was dissolved in THF (65 ml), the mixture was ice-cooled, and di-tert-butyl dicarbonate (6.85 g) was added. After stirring at room temperature for 15 hr, the reaction mixture was concentrated in vacuo. The residue was subjected to silica gel column chromatography, and the fraction eluted with ethyl acetate-hexane (1:3 to 1:1) was concentrated in vacuo to give the desired product (11.04 g) as an oil.

¹H-NMR (CDCl₃) δ 1.46 (9H, s), 1.59-1.77 (5H, m), 3.29-3.43 (7H, m), 3.98 (2H, dd), 5.09 (2H; s), 7.30-7.34 (5H, H)

MS (ESI+, m/e) 279 (M+1-“Boc”)

### Reference Example 297

### tert-Butyl (2-aminoethyl)tetrahydro-2H-pyran-4-ylcarbamate

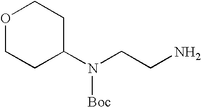

Benzyl {2-[(tert-butoxycarbonyl)(tetrahydro-2H-pyran-4-yl)amino]ethyl}carbamate (11.04 g) was dissolved in methanol (110 ml), 20% palladium hydroxide on carbon (containing 50% water) (3.5 g) was added, and a catalytic hydrogenation was performed at room temperature and atmospheric pressure for 2 days. The catalyst was filtered off, the filtrate was concentrated in vacuo, and the crystals were collected by filtration to give the desired product (6.77 g).

¹H-NMR (CDCl₃) δ 1.47 (9H, s), 1.59-1.76 (74, m), 2.80 (2H, t), 3.16 (2H, br s), 3.41 (2H, dd), 4.00 (2H, dd)

MS (ESI+, m/e) 245 (M+1)

### Reference Example 298

### 3-(Aminomethyl)-1-benzylpiperidin-3-ol

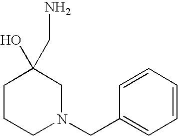

Lithium aluminum hydride (2.6 g) was suspended in diethyl ether (150 ml), 1-benzyl-3-hydroxypiperidine-3-carbonitrile (5.0 g) was added at 0° C. and the mixture was stirred at the same temperature for 1 hr and at room temperature for 1 hr. The reaction mixture was cooled to −20° C., saturated aqueous sodium bicarbonate solution was added over 30 min, and the mixture was filtrated. The filtrate was dried over anhydrous sodium sulfate, the solvent was evaporated in vacuo and the residue was vacuum dried to give the desired product (3.8 g) as an oil.

¹H-NMR (CDCl₃) δ 1.04 (4H, s), 1.64-3.25 (7H, m), 3.72-4.03 (2H, m), 4.01-4.93 (2H, m), 7.01-7.99 (5H, m)

### Reference Example 299

### tert-Butyl (2-oxopiperidin-3-yl)carbamate

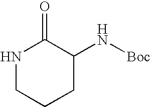

A mixture of 3-aminopiperidin-2-ol (1.55 g), di-tert-butyl dicarbonate (8.9 g), triethylamine (11.5 ml), DMF (30 ml) and methanol (30 ml) was stirred at room temperature for 3 days, and the mixture was concentrated in vacuo. The residue was subjected to silica gel column chromatography, and the fraction eluted with ethyl acetate-methanol (1:0 to 10:1) was concentrated in vacuo to give the desired product (2.80 g).

¹H-NMR (CDCl₃) δ 1.45 (9H, s), 1.52-1.68 (1H, m), 1.81-1.96 (2H, m), 2.42-2.54 (1H, m), 3,29-3.37 (2H, m), 3:97-4.09 (1H, m), 5.47 (1H, br s), 6.32 (1H, br s)

MS (ESI+, m/e) 215 (M+1)

### Reference Example 300

### tert-Butyl (2-oxo-1-phenylpiperidin-3-yl)carbamate

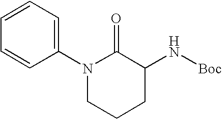

A mixture of tert-butyl (2-oxopiperidin-3-yl)carbamate (1.0 g), iodobenzene (1.96 g), copper iodide (888 mg), ethylenediamine (315 μl), potassium phosphate (3.96 g) and dioxane (20 ml) was stirred under argon atmosphere at 100° C. for 30 min and then cooled to room temperature. The reaction mixture was poured into water, and the mixture was extracted with ethyl acetate. The extract was washed with brine, and dried over anhydrous magnesium sulfate. The solvent was evaporated in vacuo. The residue was subjected to silica gel column chromatography, and the, fraction eluted with ethyl acetate-hexane (1:9 to 7:3) was concentrated in vacuo to give the desired product (764 mg).

¹H-NMR (CDCl₃) δ 1.46 (9H, s), 1.64-1.78 (1H, m), 1.99-2.09 (2H, m), 2.55-2.67 (1H, m), 3.65-3.75 (2H, m), 4.19-4.31 (1H, m), 5.54 (1H, br s), 7.21-7.28 (3H, m), 7.34-7.42 (2H, m)

MS (ESI+, m/e) 291 (M+1)

### Reference Example 301

### 3-Amino-1-phenylpiperidin-2-one trifluoroacetate

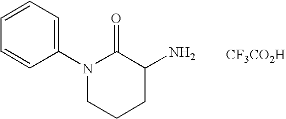

A solution of tert-butyl (2-oxo-1-phenylpiperidin-3-yl)carbamate (750 mg), TFA (10 ml) and dichloromethane (10 ml) was stirred at room temperature for 2 hr. The reaction mixture was concentrated in vacuo to give the desired product (1.68 g) as a TFA salt.

¹H-NMR (CDCl₃) δ 1.92-2.02 (2H, m), 2.04-2.09 (1H, m), 2.15-2.28 (1H, m), 3.50-3.62 (1H, m), 3.63-3.76 (1H, m), 3.83-3.96 (1H, m), 7.11-7.19 (2H, m), 7.31-7.45 (3H, m), 7.83 (2H, br s), 9.11 (1H, br s)

MS (ESI+, m/e) 191 (M+1)

### Reference Example 302

### tert-Butyl [(1S)-2-hydroxy-1-methyl-2-phenylhexyl]carbamate

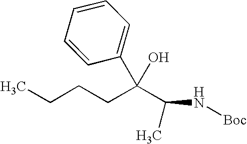

tert-Butyl [(1S)-1-methyl-2-oxo-2-phenylethyl]carbamate (2.5 g) was dissolved in THF (50 ml) and the mixture was ice-cooled. n-Butylmagnesium chloride (2 M THF solution, 10 ml) was added dropwise, and the mixture was stirred at room temperature for 3 hr. An ammonium chloride aqueous solution (20 ml) was added to the reaction mixture, and the mixture was extracted with ethyl acetate. The extract was washed successively with water and brine, and dried over anhydrous magnesium sulfate. The solvent was evaporated in vacuo. The residue was subjected to silica gel column chromatography, and the target fraction was concentrated in vacuo to give the desired product (2.10 g).

¹H-NMR (CDCl₃) δ 0.78-0.94 (5H, m), 1.15-1.31 (3H, m), 1.43-1.51 (9H, m), 1.68 (1H, s), 1.90 (2H, dt), 2.60 (1H, s), 3.93-4.07 (1H, m), 4.73 (1H, d), 7.21-7.28 (1H, m), 7.31-7.38 (4H,

### Reference Example 303

### Methyl 1-[(1S)-3-morpholino-3-oxo-1-(2-phenylethyl)propyl]-5-phenyl-1H-imidazole-4-carboxylate

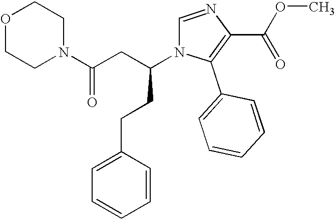

A solution of (3S)-1-morpholino-1-oxo-5-phenylpentan-3-amine (838 mg), methyl 3-bromo-2-isocyano-3-phenylacrylate (773 mg), triethylamine (588 mg) and DMF (8 ml) was stirred under argon atmosphere at room temperature for 20 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane-methanol (1:1:0 to 10:0:1) was concentrated in vacuo to give the desired product (544 mg) as an amorphous solid.

¹H-NMR (CDCl₃) δ 2.04-2.26 (2H, m), 2.46 (2H, t), 2.65 (1H, dd), 2.75 (1H, dd), 3.12-3.16 (2H, m), 3.41-3.65 (6H, m), 3.77 (3H, s), 4.48-4.53 (1H, m), 7.02 (2H, d), 7.15-7.29 (5H, m), 7.39-7.44 (3H, m), 7.69 (1H, s)

MS (ESI+, m/e) 448 (M+1)

In the same manner as in Reference Example 303, the following compounds (Reference Examples 304 to 313) were obtained.

### Reference Example 304

### Methyl 1-[(1R)-3-morpholino-3-ox6-1-(2-phenylethyl)propyl]-5-phenyl-1H-imidazole-4-carboxylate

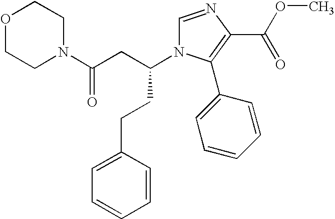

¹H-NMR (CDCl₃) δ 2.04-2.28 (2H, m), 2.46 (2H, t), 2.65 (1H, dd), 2.74 (1H, dd), 3.09-3.16 (2H, m), 3.41-3.65 (6H, m), 3.77 (3H, s), 4.46-4.55 (1H, m), 7.02 (2H, d), 7.15-7.29 (5H, m), 7.39-7.44 (3H, m), 7.69 (1H, s)

MS (ESI+, m/e) 448 (M+1)

### Reference Example 305

### Methyl 1-(1-methylpiperidin-4-yl)-5-phenyl-1H-imidazole-4-carboxylate

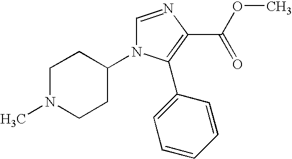

¹H-NMR (CDCl₃) δ 1.62-2.10 (6H, m), 2.26 (3H, s), 2.83-2.96 (2H, m), 3.77 (3H, s), 7.29-7.39 (2H, m), 7.41-7.53 (3H, m), 7.68 (1H, s)

### Reference Example 306

### Methyl 1-(1-benzylpiperidin-4-yl)-5-phenyl-1H-imidazole-4-carboxylate

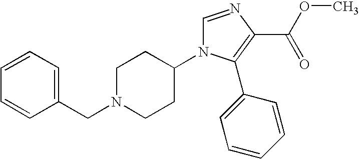

¹H-NMR (CDCl₃) δ 1.73-2.21 (6H, m), 2.93 (2H, s), 3.47 (2H, s), 3.65-3.74 (1H, m), 3.76 (3H, s), 7.20-7.36 (7H, m), 7.44-7.52 (3H, m), 7.71 (1H, s)

### Reference Example 307

### Methyl 1-[(3S)-1-benzylpyrrolidin-3-yl]-5-phenyl-1H-imidazole-4-carboxylate

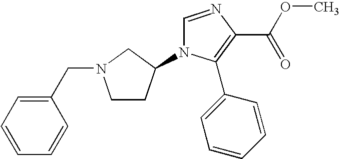

¹H-NMR (CDCl₃) δ 1.92-2.06 (1H, m), 2.26-2.42 (2H, m), 2.49 (1H, dd), 2.88-2.96 (1H, m), 3.09-3.19 (1H, m), 3.54 (1H, d), 3.76 (1H, d), 3.77 (3H, s), 4.33-4.42 (1H, m), 7.23-7.35 (7H, m), 7.42-7.50 (3H, m), 8.08 (1H, s)

### Reference Example 308

### Methyl 1-[(3R)-1-benzylpyrrolidin-3-yl]-5-phenyl-1H-imidazole-4-carboxylate

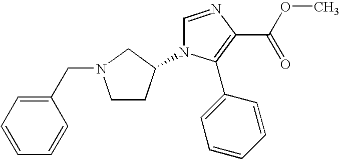

¹H-NMR (CDCl₃) δ 1.92-2.06 (1H, m), 2.26-2.42 (2H, m), 2.49 (1H, dd), 2.88-2.96 (1H, m), 3.09-3.19 (1H, m), 3.54 (1H, d), 3.76 (1H, d), 3.77 (3H, s), 4.33-4.42 (1H, m), 7.23-7.35 (7H, m), 7.42-7.50 (3H, m), 8.08 (1H, s)

### Reference Example 309

### Methyl 1-(1-benzylpyrrolidin-3-yl)-5-phenyl-1H-imidazole-4-carboxylate

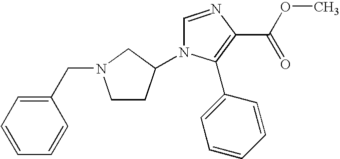

¹H-NMR (CDCl₃) δ 1.92-2.06 (1H, m), 2.26-2.42 (2H, m), 2.49 (1H, dd), 2.88-2.96 (1H, m), 3.09-3.19 (1H, m), 3.54 (1H, d), 3.76 (1H, d), 3.77 (3H, s), 4.33-4.42 (1H, m), 7.23-7.35 (7H, m), 7.42-7.50 (3H, m), 8.08 (1H, s)

### Reference Example 310

### Methyl 5-phenyl-1-(1,2,3,4-tetrahydronaphthalen-2-yl)-1H-imidazole-4-carboxylate

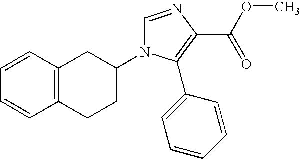

¹H-NMR (CDCl₃) δ 2.09-2.24 (2H, m), 2.82-2.97 (2H, m), 3.13 (2H, d), 3.78 (3H, s), 4.19-4.32 (1H, m), 7.02-7.17 (4H, m), 7.34-7.42 (2H, m), 7.43-7.52 (3H, m), 7.65 (1H, s)

### Reference Example 311

### Methyl 1-(2-hydroxy-2-phenylethyl)-5-phenyl-1H-imidazole-4-carboxylate

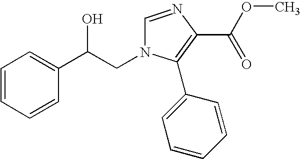

¹H-NMR (CDCl₃) δ 3.67 (3H, s), 3.91-4.06 (2H, m), 4.26 (1H, br s), 4.80 (1H, d), 7.02-7.11 (2H, m), 7.19-7.32 (5H, m), 7.39-7.52 (3H, m), 7.72 (1H, s)

### Reference Example 312

### Methyl-(2-hydroxy-2-pyridin-2-ylethyl)-5-phenyl-1H-imidazole-4-carboxylate

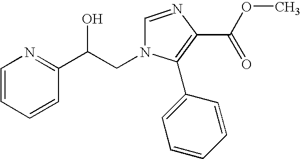

¹H-NMR (CDCl₃) δ 3.73 (3H, s), 4.00-4.30 (2H, m), 4.80 (1H, s), 6.81-6.94 (1H, m), 7.13-7.30 (3H, m), 7.38-7.51 (3H, m), 7.58 (1H, td), 7.63 (1H, s), 8.47 (1H, d)

### Reference Example 313

### Methyl 1-((1S,2R)-2-hydroxy-1,2-diphenylethyl]-5-phenyl-1H-imidazole-4-carboxylate

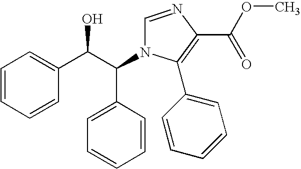

¹H-NMR (CDCl₃) δ 3.50 (3H, s), 4.86 (1H, d), 5.26 (1H, d), 6.68 (2H, d), 6.82-6,96 (3H, m), 7.02-7.11 (5H, m), 7.13-7.27 (5H, m), 7.95 (1H, s)

### Reference Example 314

### tert-Butyl 3-[4-(methoxycarbonyl)-5-phenyl-1H-imidazol-1-yl]piperidine-1-carboxylate

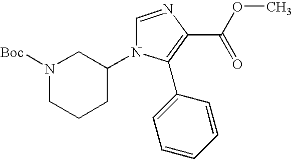

tert-Butyl 3-aminopiperidine-1-carboxylate (5.01 g) and triethylamine (10.5 ml) were dissolved in DMF (50 ml) and the mixture was ice-cooled. Methyl 3-bromo-2-isocyano-3-phenylacrylate (6.65 g) was added thereto and the mixture was stirred at room temperature for 3 days. DBU (3.74 ml) was added to the reaction mixture and the mixture was stirred at room temperature for additional 3 hr. The reaction mixture was concentrated in vacuo, and the residue was partitioned between ethyl acetate and water. The organic layer was washed successively with a 10% aqueous citric acid solution, saturated aqueous sodium bicarbonate solution and brine, and dried over anhydrous sodium, sulfate, and the solvent was evaporated in vacuo. The residue was subjected to silica gel column chromatography, and the fraction eluted with ethyl acetate-methanol (1:0 to 9:1) was concentrated in vacuo to give the desired product (7.03 g).

¹H-NMR (CDCl₃) δ 1.36-1.49 (1H, m), 1.40 (9H, s), 1.81 (2H, s), 1.98-2.13 (1H, m), 2.80 (1H, t), 3.01 (1H, t), 3.74-3.87 (4H, m), 3.89-4.04 (1H, m), 4.04-4.19 (1H, m), 7.35 (2H, s), 7.45-7.54 (3H, m), 7.68 (1H, s)

### Reference Example 315

### Methyl 1-(2-oxopiperidin-1-yl)-5-phenyl-1H-imidazole-4-carboxylate

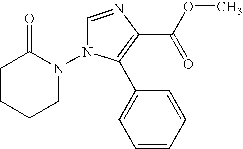

A solution of 1-aminopiperidin-2-one (685 mg), N,N-diisopropylethylamine (10 ml) and triethylamine (1.7 ml) in DMF (15 ml) was ice-cooled, methyl 3-bromo-2-isocyano-3-phenylacrylate (1.06 g) was added thereto and the mixture was stirred at room temperature for 3 days. DBU (0.60 ml) was added thereto and the mixture was stirred at room temperature for 2 days. The reaction mixture was concentrated in vacuo, and poured into saturated aqueous sodium bicarbonate solution, and the mixture was extracted with ethyl acetate. The extract was washed successively with a 10% aqueous citric acid solution and brine, and dried over anhydrous sodium sulfate, and the solvent was evaporated in vacuo. The residue was subjected to silica gel column chromatography, and,the fraction eluted with ethyl acetate-methanol (8:2) was concentrated in vacuo to give the desired product (312 mg) as an oil.

NMR (CDCl₃) δ 1.46-1.72 (2H, m), 1.72-1.96 (2H, m), 2.24-2.65 (2H; m), 3.09-3.27 (1H, m), 3.49-3.70 (1H, m), 3.81 (3H, s), 7.35-7.52 (5H, m), 7.59 (1H, s)

### Reference Example 316

### Ethyl 1-((2R)-2-{[(benzyloxy)carbonyl]amino}-3-morpholino-3-oxopropyl)-5-phenyl-1H-imidazole-4-carboxylate

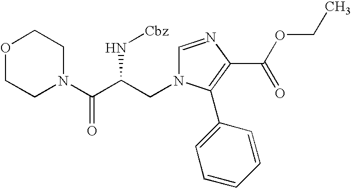

A solution of benzyl [(1R)-1-(aminomethyl)-2-morpholino-2-oxoethyl]carbamate (4.10 g), ethyl 3-bromo-2-isocyano-3-phenylacrylate (3.74 g), triethylamine (2.70 g) and DMF (35 ml) was stirred under argon atmosphere at room temperature for 2 days, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane-methanol (1:1:0 to 20:0:1) was concentrated in vacuo to give the desired product (4.08 g) as an amorphous solid.

¹H-NMR (CDCl₃) δ 1.20 (3H, t), 2.47-2.53 (1H, m), 2.73-2.79 (1H, m), 3.19-3.24 (1H, m), 3.32-3.36 (1H, m), 3.45-3.59 (4H, m), 3.85 (1H, dd), 4.04 (1H, dd), 4.21 (2H, q), 4.59 (1H, dt), 4.96 (1H, d), 5.02 (1H, d), 5.76 (1H, d), 7.21-7.38 (5H, m), 7.50-7.51 (6H, m)

MS (ESI+, m/e) 507 (M+1)

In the same manner as in Reference Example 316, the following compounds (Reference Examples 317 to 336) were obtained.

### Reference Example 317

### Ethyl 1-{(1R)-1-[2-(methylthio)ethyl]-3-morpholino-3-oxopropyl}-5-phenyl-1H-imidazole-4-carboxylate

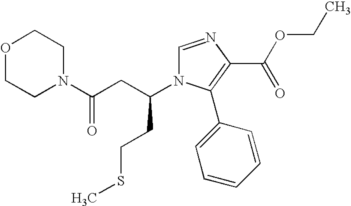

¹H-NMR (CDCl₃) δ 1.21 (3H, t), 2.00 (3H, s), 2.05-2.14 (2H, M), 2.23-2.34 (2H, m), 2.67 (1H, dd), 2.77 (1H, dd), 3.20-3.23 (2H, m), 3.48-3.65 (6H, m), 4.22 (2H, q), 4.64-4.69 (1H, m), 7.38-7.41 (2H, m), 7.47-7.51 (3H, m), 7.67 (1H, s)

MS (ESI+, m/e) 432 (M+1)

### Reference Example 318

### Ethyl 1-[(1S)-3-[(2-furylmethyl)amino]-1-(2-morpholino-2-oxoethyl)-3-oxopropyl]-5-phenyl-1H-imidazole-4-carboxylate

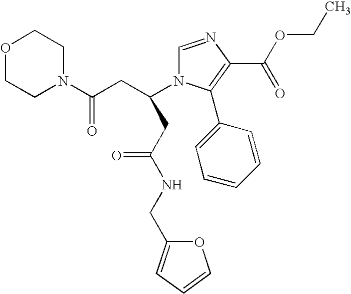

¹H-NMR (CDCl₃) δ 1.16 (3H, t), 2.71-2.93 (4H, m), 3.12-3.14 (2H, m), 3.42-3.65 (6H, m), 4.17 (2H, q), 4.34-4.35 (2H, m), 4.87 (1H, quintet), 6.16 (1H, dd), 6.28 (1H, dd), 6.49 (1H, t), 7.32 (1H, dd), 7.38-7.50 (5H, m), 7.73 (1H, s)

MS (ESI+, m/e) 495 (M+1)

### Reference Example 319

### Ethyl 1-{2-[(tert-butoxycarbonyl)(tetrahydro-2H-pyran-4-yl)amino]ethyl}-5-phenyl-1H-imidazole-4-carboxylate

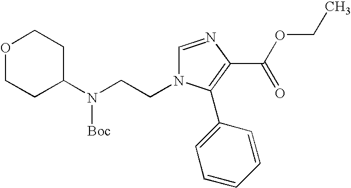

¹H-NMR (CDCl₃) δ 1.09-1.28 (8H, m), 1.45 (9H, s), 3.09 (2H, t), 3.27 (2H, br t), 3.83 (2H, d), 3.98 (2H, br s), 4.23 (2H, q), 7.37-7.39 (2H, m), 7.49-7.52 (4H, m)

MS (ESI+, m/e) 444 (M+1)

### Reference Example 320

### Ethyl 1-(2-oxoazepan-3-yl)-5-phenyl-1H-imidazole-4-carboxylate

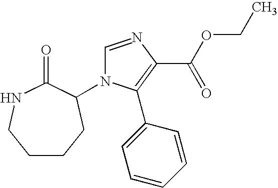

¹H-NMR (CDCl₃) δ 1.22 (3H, t), 1.45-1.57 (2H, m), 1.76-1.89 (2H, m), 1.99-2.32 (2H, m), 2.85-2.99 (1H, m), 3.20 (1H, s), 4.23 (2H, q), 4.59 (1H, d), 6.48 (1H, br s), 7.30 (2H, s), 7.45 (3H, s), 7.75 (1H, s)

### Reference Example 321

### Ethyl 1-(2-oxotetrahydrofuran-3-yl)-5-phenyl-1H-imidazole-4-carboxylate

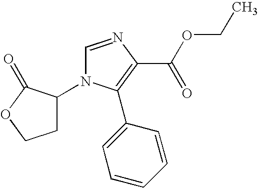

¹H-NMR (CDCl₃) δ 1.21 (3H, t), 2.56 (1H, s), 2.60-2.73 (1H, m), 4.17-4.31 (3H, m), 4.47 (1H, td), 4.80 (1H, dd), 7.35-7.46 (2H, m), 7.49 (3H, s), 7.65 (1H, s)

### Reference Example 322

### Ethyl 1-[(1-methylpiperidin-2-yl)methyl]-5-phenyl-1H-imidazole-4-carboxylate

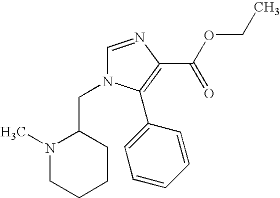

¹H-NMR (CDCl₃) δ 0.97-1.13 (2H, m), 1.17-1.31 (4H, m), 1.40-1.46 (1H, m), 1.48-1.53 (1H, m), 1.56-1.69 (1H, m), 1.90-1.98 (2H, m), 2.05 (3H, s), 2.68-2.79 (1H, m), 3.65-3.78 (1H, m), 4.07-4.15 (1H, m), 4.21 (2H, q), 7.29-7.37 (2H, m), 7.41-7.51 (3H, m), 7.64 (1H, s)

MS (ESI+, m/e) 328 (M+1)

### Reference Example 323

### Ethyl 1-[(1S,2S)-2-hydroxy-1-(methoxymethyl)-2-phenylethyl]-5-phenyl-1H-imidazole-4-carboxylate

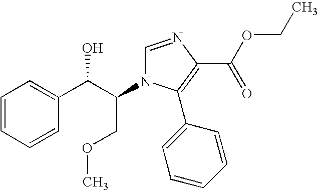

¹H-NMR (CDCl₃) δ 1.16 (3H, t), 2.99-3.10 (1H, m), 3.33 (3H, s), 3.36-3.43 (1H, m), 3.62-3.78 (1H, m), 4.02-4.18 (3H, m), 4.59 (1H, d), 5.10 (1H, d), 6.92-6.99 (1H, m), 7.20-7.36 (8H, m), 8.13 (1H, s)

MS (ESI+, m/e) 381 (M+1)

### Reference Example 324

### Ethyl 1-[(1R)-1-benzyl-2-hydroxyethyl]-5-phenyl-1H-imidazole-4-carboxylate

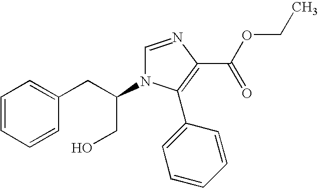

¹H-NMR (CDCl₃) δ 1.07 (3H, t), 3.10 (2H, d), 3.81-3.95 (2H, m), 4.03-4.15 (3H, m), 6.79-6.93 (3H, m), 7.18-7.23 (3H, m), 7.26-7.38 (4H, m), 7.94 (1H, s)

MS (ESI+, m/e) 351 (M+1)

### Reference Example 325

### tert-Butyl 2-{[4-(ethoxycarbonyl)-5-phenyl-1H-imidazol-1-yl]methyl}piperidine-1-carboxylate

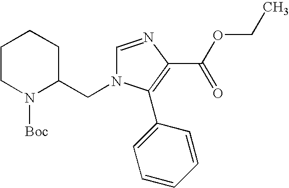

¹H-NMR (CDCl₃) δ 1.20 (3H, t), 1.37 (9H, br s), 1.43-1.51 (7H, m), 3.81-3.93 (2H, m), 4.03-4.13 (1H, m), 4.21 (2H, q), 4.40 (1H, br s), 7.33-7.39 (2H, m), 7.42-7.51 (3H, m), 7.57 (1H, s)

MS (ESI+, m/e) 414 (M+1)

### Reference Example 326

### Ethyl 1-[(1S,2S)-2-hydroxy-1-(hydroxymethyl).-2-phenylethyl]-5-phenyl-1H-imidazole-4-carboxylate

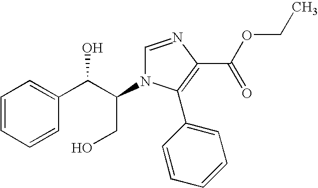

¹H-NMR (CDCl₃) δ 0.92 (3H, t), 3.90-4.03 (4H, m), 4.04-4.18 (1H, m), 5.20 (1H, s), 6.90 (4H, d), 7.09-7.23 (6H, m), 7.23-7.35 (2H, m), 8.21-8.28 (1H, m)

### Reference Example 327

### Ethyl 1-[(1S)-2-hydroxy-2-methyl-1-phenylpropyl]-5-phenyl-1H-imidazole-4-carboxylate

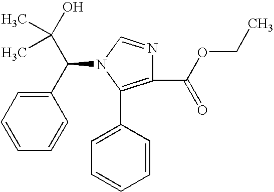

MS (ESI+, m/e) 365 (M+1)

### Reference Example 328

### Ethyl 1-[(1S)-2-ethyl-2-hydroxy-1-phenylbutyl]-5-phenyl-1H-imidazole-4-carboxylate

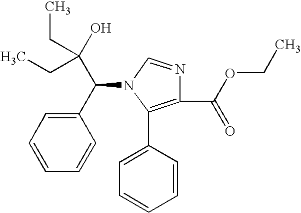

¹H-NMR (CDCl₃) δ 0.69 (6H, td), 1.15-1.28 (3H, m), 1.37-1.52 (2H, m), 1.65-1.79 (3H, m), 4.19 (2H, dq), 7.20-7.27 (3H, m), 7.31-7.36 (4H, m), 7.39-7.48 (3H, m), 8.59 (1H, s)

### Reference Example 329

### Ethyl 1-[(1S)-2-butyl-2-hydroxy-1-phenylhexyl]-5-phenyl-1H-imidazole-4-carboxylate

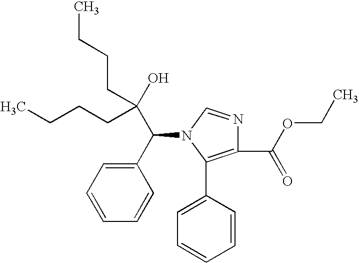

¹H-NMR (CDCl₃) δ 0.73-0.87 (6H, m), 1.06-1.22 (12H, m), 1.28-1.35 (2H, m), 4.12-4.25 (2H, m), 7.08-7.20 (2H, m), 7.22 (2H, dd), 7.32-7.42 (6H, m), 7.44-7.50 (2H, m), 8.60 (1H, s)

### Reference Example 330

### Ethyl 1-[(1S)-2-hydroxy-1,2-dimethylpropyl]-5-phenyl-1H-imidazole-4-carboxylate

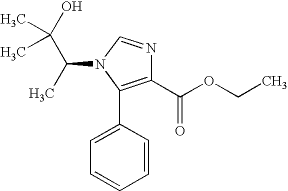

¹H-NMR (CDCl₃) δ 0.91 (3H, s), 1.16-1.26 (6H, m), 1.50-1.56 (3H, m), 3.82 (1H, q), 4.16-4.27 (2H, m), 7.27-7.37 (2H, m), 7.47 (3H, td), 7.95 (1H, s)

### Reference Example 331

### tert-Butyl trans-3-[4-(ethoxycarbonyl)-5-phenyl-1H-imidazol-1-yl]-4-hydroxypiperidine-1-carboxylate

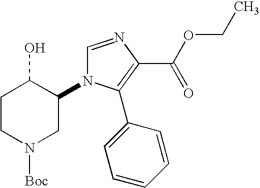

¹H-NMR (CDCl₃) δ 1.10-1.19 (3H, m), 1.36 (9H, br s), 1.42-1.82 (2H, m), 2.05 (1H, s), 2.74-2.89 (2H, m), 3.69 (1H, s), 3.97-4.26 (5H, m), 7.42-7.54 (6H, m)

### Reference Example 332

### Ethyl 1-(2-hydroxy-2-methylpropyl)-5-phenyl-1H-imidazole-4-carboxylate

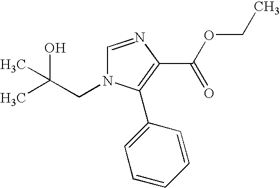

¹H-NMR (CDCl₃) δ 1.03 (6H, s), 1.21 (3H, t), 2.04 (1H, br s), 3.82 (2H, s), 4.21 (2H, q), 7.29-7.37 (2H, m), 7.42-7.47 (3H, m), 7.87 (1H, s)

### Reference Example 333

### Ethyl 1-[(1-benzyl-3-hydroxypiperidin-3-yl)methyl]-5-phenyl-1H-imidazole-4-carboxylate

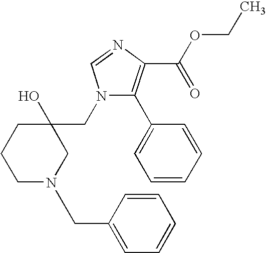

¹H-NMR (CDCl₃) δ 1.24 (3H, t), 1.34-2.15 (9H, m), 2.53-2.80 (2H, m), 3.52 (4H, q), 7.06-7.55 (11H, m)

### Reference Example 334

### Ethyl 1-[(1-benzyl-4-hydroxypiperidin-4-yl)methyl]-5-phenyl-1H-imidazole-4-carboxylate

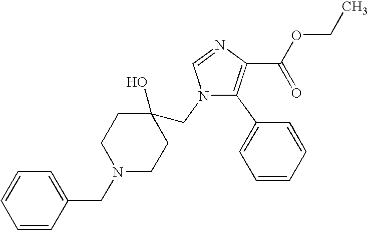

¹H-NMR (CDCl₃) δ 1.19-1.89 (7H, m), 2.21-2.40 (3H, m), 2.64-2.73 (1H, m), 2.89 (1H, s), 2.96 (1H, s), 3.10-3.54 (3H, m), 4.24 (2H, q), 6.99-7.89 (11H, m)

### Reference Example 335

### Ethyl 1-[(4-hydroxytetrahydro-2H-pyran-4-yl)methyl]-5-phenyl-1H-imidazole-4-carboxylate

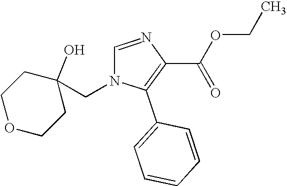

¹H-NMR (CDCl₃) δ 1.16-1.47 (5H, m), 1.52-1.75 (2H, m), 2.01 (1H, br s), 3.50-3.70 (4H, m), 3.90 (2H, s), 4.23 (2H, q), 7.30-7.35 (2H, m), 7.42-7.53 (3H, m), 7.81 (1H, s)

MS (ESI+, m/e) 331 (M+1)

### Reference Example 336

### Ethyl 5-phenyl-1-(4-hydroxytetrahydro-2H-pyran-3-yl)-1H-imidazole-4-carboxylate

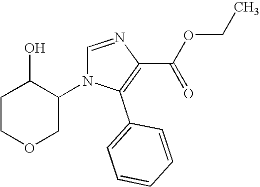

¹H-NMR (CDCl₃) δ 0.94-1.17 (3H, m), 1.55-2.23 (2H, m), 3.29-3.57 (1H, m), 3.60-4.37 (7H, m), 3.71 (1H, dd), 7.13-7.30 (1H, m), 7.35-7.49 (4H, m), 7.54-7.84 (1H, m)

### Reference Example 337

### Ethyl 1-[(1R,2S)-2-hydroxy-1,2-diphenylethyl]-5-phenyl-1H-imidazole-4-carboxylate

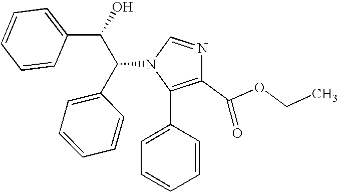

(1S,2S)-2-Amino-1,2-diphenylethanol (0.56 g) and DMAP (0.96 g) were dissolved in DMF (10 ml) and the mixture was ice-cooled. Ethyl 3-bromo-2-isocyano-3-phenylacrylate (0.70 g) was added thereto and the mixture was stirred at room temperature for 12 hr. The reaction mixture was poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the target fraction was concentrated in vacuo to give the desired product (0.54 g).

¹H-NMR (CDCl₃) δ 1.11 (3H, t), 4.03-4.18 (2H, m), 5.02 (1H, d), 5.40 (1H, s), 6.83 (2H, d), 6.98-7.12 (3H, m), 7.14-7.25 (5H, m), 7.33-7.44 (5H, m), 8.09 (1H, s)

In the same manner as in Reference Example 337, the following compound (Reference Example 338) was obtained.

### Reference Example 338

### Ethyl 1-(1-benzothien-5-yl)-5-phenyl-1H-imidazole-4-carboxylate

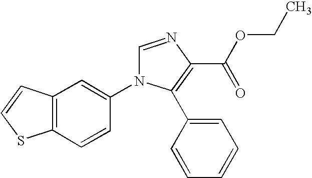

MS (ESI+, m/e) 349 (M+1)

### Reference Example 339

### Ethyl 1-(2-oxo-1-phenylpiperidin-3-yl)-5-phenyl-1H-imidazole-4-carboxylate

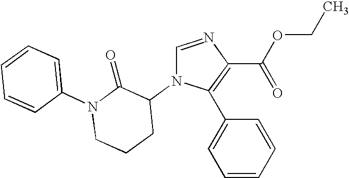

A mixture of 3-amino-1-phenylpiperidin-2-one trifluoroacetate (810 mg), ethyl 3-bromo-2-isocyano-3-phenylacrylate (500 mg) and N,N-diisopropylethylamine (1.9 ml) was stirred at room temperature for 13 hr. The reaction mixture was poured into brine, and the mixture was extracted with ethyl acetate. The extract was dried over anhydrous magnesium sulfate, and the solvent was evaporated in vacuo. The residue was subjected to silica gel column chromatography, and the fraction eluted with ethyl acetate-methanol (1:0 to 3:1) was concentrated in vacuo to give the desired product (248 mg).

¹H-NMR (CDCl₃) δ 1.23 (3H, t), 1.95-2.10 (2H, m), 2.19-2.34 (2H, m), 3.57-3.73 (2H, m), 4.20 (2H, q), 4.63-4.73 (1H, m), 7.18-7.31 (4H, m), 7.37-7.50 (6H, m), 7.66 (1H, s)

MS (ESI+, m/e) 390 (M+1)

### Reference Example 340

### Ethyl 1-[(1S)-2-hydroxy-1-methyl-2-phenylhexyl)-5-phenyl-1H-imidazole-4-carboxylate

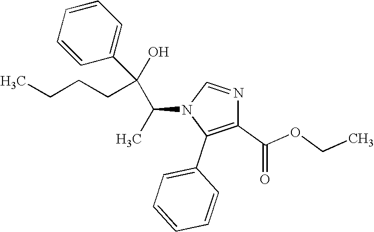

tert-Butyl [(1S)-2-hydroxy-1-methyl-2-phenylhexyl]carbamate (1.50 g) was dissolved in ethyl acetate (10 ml), a 4 N hydrogen chloride-ethyl acetate solution was added, and the mixture was stirred at room temperature for 30 min. The reaction mixture was concentrated in vacuo, and the residue and triethylamine (2.70 ml) were dissolved in DMF (20 ml). The mixture was ice-cooled, ethyl 3-bromo-2-isocyano-3-phenylacrylate (1.40 g) was added and the mixture was stirred at room temperature for 12 hr. By treating in the same manner as in Reference Example 316, the desired product (0.80 g) was obtained.

¹H-NMR (CDCl₃) δ 0.60 (1H, d), 0.64-0.78 (3H, m), 0.89-0.95 (1H, m), 1.02-1.16 (3H, m), 1.20-1.31 (6H, m), 1.36-1.52 (1H, m), 1.73 (1H, s), 4.18-4.33 (3H, m), 7.05 (2H, d), 7.22-7.37 (5H, m), 7.49-7.58 (3H, m), 7.97-8.11 (1H, m)

### Reference Example 341

### Methyl 1-(2,3-dihydro-1H-inden-2-yl)-5-[3-(3-methoxypropoxy)phenyl]-1H-imidazole-4-carboxylate

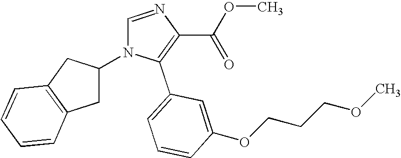

Methyl 3-bromo-2-(formylamino)-3-[3-(3-methoxypropoxy)phenyl]acrylate (2.98 g) and triethylamine (2.02 g) were dissolved in dichloromethane (25 ml) and the mixture was ice-cooled. Phosphoryl chloride (1.35 g) was added, and the mixture was stirred at 0° C. for 2 hr. The reaction mixture was poured into saturated aqueous sodium bicarbonate solution and, after vigorously stirring at room temperature for 1 hr, the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:3) was concentrated in vacuo to give methyl 3-bromo2-isocyano-3-[3-(3-methoxypropoxy)phenyl]acrylate (2.32 g) as an oil. The total amount thereof and indan-2-amine (1.05 g) were dissolved in DMF (20 ml), triethylamine (1.33 g) was added, and the mixture was stirred under argon atmosphere at room temperature for 15 hr. The reaction mixture was poured into saturated aqueous sodium bicarbonate solution, and the mixture was extracted with ethyl acetate. The extract was, washed successively with water and brine, and dried over anhydrous magnesium sulfate. The solvent was evaporated in vacuo. The residue was subjected to silica gel column chromatography, and the fraction eluted with ethyl acetate-hexane (1:2 to 1:0) was concentrated in vacuo to give the desired product (2.03 g) as an oil.

¹H-NMR (CDCl₃) δ 2.07 (2H, quintet), 3.29 (2H, dd), 3.36 (3H, s), 3.37 (2H, dd), 3.57 (2H, t), 3.77 (3H, s), 4.08 (2H, t), 4.75-4.80 (1H, m), 6.90-7.02 (3H, m), 7.23 (4H, s), 7.39 (1H, t), 7.44 (1H, s)

MS (ESI+, m/e) 407 (M+1)

### Reference Example 342

### Methyl 5-cyclohexyl-1-phenyl-1H-imidazole-4-carboxylate

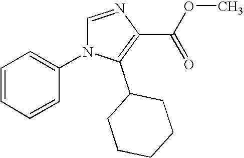

Sodium hydride (60% in oil) (4.8 g) was suspended in THF (100 ml) and the mixture was ice-cooled. A solution of methyl isocyanoacetate (10 g) and cyclohexanecarbaldehyde (13.5 ml) in THF (20 ml) was added dropwise. After the completion of the dropwise addition, the mixture was stirred at room temperature for 3 hr. The reaction mixture was ice-cooled, and acetic acid (20 ml) was slowly added. The mixture was poured into ice-water, and the mixture was extracted with ethyl acetate. The extract was washed successively with water, saturated aqueous sodium bicarbonate solution, water and brine, dried over anhydrous magnesium sulfate, and concentrated in vacuo. The residue was subjected to silica gel column chromatography, and the fraction eluted with ethyl acetate-hexane (7:3) was concentrated in vacuo to give methyl 3-cyclohexyl-2-(formylamino)acrylate (17.8 g) as an oil. The total amount thereof was dissolved in a mixture of carbon tetrachloride (150 ml) and chloroform (50 ml) and the mixture was ice-cooled. NBS (15.8 g) was added and the mixture was stirred at 0° C. for 1.5 hr and at room temperature for 3 hr. The reaction mixture was ice-cooled again, triethylamine (12.3 ml) was added, and the mixture was stirred at 0° C. for 20 min and at room temperature for 40 min. The reaction mixture was washed successively with water and brine, dried over anhydrous magnesium sulfate, and concentrated in vacuo. The residue was subjected to silica gel column chromatography, and the fraction eluted with ethyl acetate-hexane (35:65) was concentrated in vacuo to give methyl 3-bromo-3-cyclohexyl-2-(formylamino)acrylate (14.8 g) as an oil. The total amount thereof and triethylamine (17.8 ml) were dissolved in dichloromethane (120 ml) and the mixture was ice-cooled. Phosphoryl chloride (5.2 ml) was added dropwise. The reaction mixture was stirred at 0° C. for 3 hr, and poured into ice-cooled 10% aqueous potassium carbonate solution (120 ml), and the mixture was vigorously stirred at room temperature for 2 hr. The organic layer was separated, washed successively with water and brine, dried over anhydrous magnesium sulfate, and concentrated in vacuo. The residue was subjected to silica gel column chromatography, and the fraction eluted with ethyl acetate-hexane (15:85) was concentrated in vacuo to give methyl 3-bromo-3-cyclohexyl-2-isocyanoacrylate (9.17 g) as an oil. A 1.0 g portion thereof was added to a solution of aniline (0.34 ml) and triethylamine (1.5 ml) in DMF (10 ml) at 0° C., and the mixture was stirred at room temperature for 12 hr. The reaction mixture was poured into saturated aqueous sodium bicarbonate solution, and the mixture was extracted with ethyl acetate. The extract was washed successively with water and brine, and dried over anhydrous sodium sulfate, and, the solvent was evaporated in vacuo. The residue was subjected to silica gel column chromatography, and the fraction eluted with ethyl acetate was concentrated in vacuo to give the desired product (934 mg) as an oil.

¹H-NMR (CDCl₃) δ 1.11-1.26 OH, m), 1.53-1.79 (5H, m), 1.90-2.11 (2H, m), 2.83-2.95 (1H, m), 3.93 (3H, s), 7.23-7.30 (2H, m), 7.43 (1H, s), 7.48-7.60 (3H, m)

MS (ESI+, m/e) 285 (M+1)

In the same manner as in Reference Example 342, the following compound (Reference Example 343) was obtained.

### Reference Example 343

### Methyl 5-cyclopropyl-1-phenyl-1H-imidazole-4-carboxylate

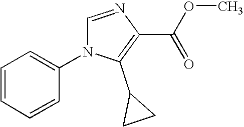

¹H-NMR (CDCl₃) δ 0.47-0.65 (2H, m), 0.75-0.98 (2H, m), 1.77-1.96 (1H, m), 3.93 (3H, s), 7.30-7.42 (2H, in), 7.42-7.60 (4H, m)

MS (ESI+, m/e) 243 (M+1)

### Reference Example 344

### Methyl (5-phenyl-1-piperidin-3-yl-1H-imidazole-4-carboxylate hydrochloride

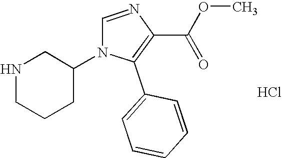

A 4 N hydrogen chloride-ethyl acetate solution (50 ml) was added to tert-butyl 3-[4-(methoxycarbonyl)-5-phenyl-1H-imidazol-1-yl]piperidine-1-carboxylate (7.00 g). After stirring at room temperature for 12 hr, the reaction mixture was concentrated in vacuo to give the desired product (6.56 g) as an amorphous solid.

¹H-NMR (DMSO-d₆) δ 1.57-1.73 (1H, m), 1.89 (1H, d), 1.99-2.27 (2H, m), 2.79 (1H, q), 3.14-3.23 (1H, m), 3.39-3.54 (2H, m), 3.66 (3H, s), 4.31 (1H, t), 7.48-7.61 (5H, m), 9.29 (1H, br s), 9.47 (1H, d), 10.17 (1H, d)

MS (ESI+, m/e) 286 (M+1)

In the same manner as in Reference Example 344, the following compound (Reference Example 345) was obtained.

### Reference Example 345

### Ethyl 1-[trans-4-hydroxypiperidin-3-yl]-5-phenyl-1H-imidazole-4-carboxylate hydrochloride

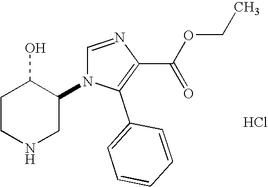

¹H-NMR (DMSO-d₆) δ 1.04 (3H, t), 1.58 (1H, s), 2.05 (1H, dd), 2.95 (1H, s), 3.27 (1H, d), 3.47 (2H, s), 3.95-4.26 (4H, m), 7.45-7.57 (5H, m), 9.12 (1H, br s), 9.18 (1H, s), 9.92 (1H, d)

MS (ESI+, m/e) 316 (M+1)

### Reference Example 346

### Ethyl 5-phenyl-1-(piperidin-2-ylmethyl)-1H-imidazole-4-carboxylate

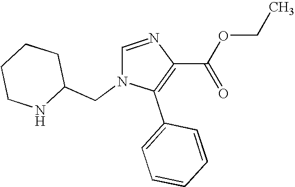

A mixture of tert-butyl 2-([4-(ethoxycarbonyl)-5-phenyl-1H-imidazol-1-yl]methyl)piperidine-1-carboxylate (1.58 g), TFA (15 ml) and dichloromethane (15 ml) was stirred at room temperature for 3 hr, and concentrated in vacuo. The residue was dissolved in ethyl acetate, and the solution was washed successively with saturated aqueous sodium bicarbonate solution and brine, and dried over anhydrous magnesium sulfate. The solvent was evaporated in vacuo to give the desired product (950 mg).

MS (ESI+, m/e) 314 (M+1)

### Reference Example 347

### Benzyl 3-[4-(methoxycarbonyl)-5-phenyl-1H-imidazol-1-yl]piperidine-1-carboxylate

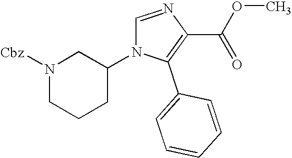

Methyl 5-phenyl-1-piperidin-3-yl-1H-imidazole-4-carboxylate hydrochloride (5.79 g) and triethylamine (7.53 ml) were suspended in THF (200 ml) and the mixture was ice-cooled. Benzyl chloroformate (3.08 ml) was added and the mixture was stirred at room temperature for 12 hr. The reaction mixture was poured into water, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-methanol (9:1) was concentrated in vacuo to give the desired product (6.66 g) as an amorphous solid.

¹H-NMR (CDCl₃) δ 1.42-1.56 (1H, m), 1.73-1.98 (2H, m), 2.06-2.20 (1H, m), 2.76-2.91 (1H, m), 2.94-3.08 (1H, m), 3.76 (3H, s), 3.76-3.89 (1H, m), 4.00-4.41 (2H, m), 4.97-5.12 (2H, m), 7.17-7.53 (10H, m), 7.66 (1H, s)

MS (ESI+, m/e) 420 (M+1)

### Reference Example 348

### Ethyl 1-[trans-4-hydroxy-1-(phenylsulfonyl)piperidin-3-yl]-5-phenyl-1H-imidazole-4-carboxylate

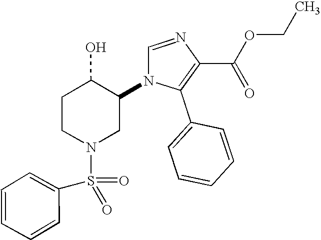

A mixture of ethyl 1-[trans-4-hydroxypiperidin-3-yl]-5-phenyl-1H-imidazole-4-carboxylate hydrochloride (500 mg), triethylamine and THF (15 ml) was ice-cooled, benzenesulfonyl chloride (0.22 ml) was added and the mixture was stirred at room temperature for 7 hr. The reaction mixture was poured into aqueous sodium bicarbonate solution, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate was concentrated in vacuo to give the desired product (429 mg) as an amorphous solid.

¹H-NMR (CDCl₃) δ 1.12 (3H, t), 2.08 (1H, s), 2.43-2.55 (2H, m), 3.75-3.95 (5H, m), 4.15 (2H, d), 7.35-7.43 (3H, m), 7.46-7.54 (5H, m), 7.58-7.67 (3H, m)

MS (ESI+, m/e) 456 (M+1)

### Reference Example 349

### Benzyl trans-3-[4-(ethoxycarbonyl)-5-phenyl-1H-imidazol-1-yl]-4-hydroxypiperidine-1-carboxylate

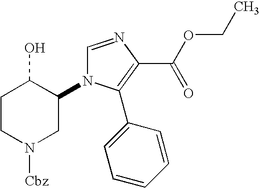

Ethyl 1-[trans-4-hydroxypiperidin-3-yl]-5-phenyl-1H-imidazole-4-carboxylate hydrochloride (500 mg) was dissolved in THF-water (1:1, 20 ml), benzyl chloroformate (0.24 ml) and potassium carbonate (360 mg) were added and the mixture was stirred at room temperature for 12 hr. The reaction mixture was extracted with ethyl acetate, the extract was washed with brine, and dried over anhydrous sodium sulfate, and the solvent Was evaporated in vacuo. The residue was subjected to silica gel column chromatography, and the fraction eluted with ethyl acetate was concentrated in vacuo to give the desired product (527 mg) as an amorphous solid.

¹H-NMR (CDCl₃) δ 1.12 (3H, t), 1.54 (1H, s), 2.10 (2H, s), 2.88 (2H, s), 3.64-3.78 (1H, m), 3.95-4.42 (5H, m), 5.03 (2H, br s), 7.16-7.58 (11H, m)

MS (ESI+, m/e) 450 (M+1)

### Reference Example 350

### Benzyl 3-[4-(ethoxycarbonyl)-5-phenyl-1H-imidazol-1-yl]-4-oxopiperidine-1-carboxylate

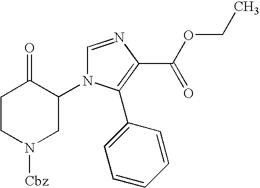

Benzyl trans-3-(4-(ethoxycarbonyl)-5-phenyl-1H-imidazol-1-yl]-4-hydroxypiperidine-1-carboxylate (500 mg) and triethylamine (0.46 ml) were dissolved in DMSO (10 ml) and the solution was ice-cooled. Pyridine.sulfur trioxide complex (525 mg) was added and the mixture was stirred at room temperature for 2 hr. The reaction mixture was poured into water, and the mixture was extracted with ethyl acetate. The extract was washed successively with a 10% aqueous citric acid solution, saturated aqueous sodium bicarbonate solution and brine, and dried over anhydrous sodium sulfate, and the solvent was evaporated in vacuo. The residue was subjected to silica gel column chromatography, and the fraction eluted with ethyl acetate was concentrated in vacuo to give the desired product (396 mg) as an amorphous solid.

(CDCl₃) δ 1.21 (3H, t), 2.48-2.63 (2H, m), 3.13-3.27 (1H, m), 3.35 (1H, t), 4.22 (2H, q), 4.39-4.81 (3H, m), 5.10 (2H, s), 7.18-7.50 (10H, m), 7.53 (1H, s)

MS (ESI+, m/e) 448 (M+1)

### Reference Example 351

### Benzyl 4-[4-(ethoxycarbonyl)-5-phenyl-1H-imidazol-1-yl]-1-oxa-6-azaspiro[2.5]octane-6-carboxylate

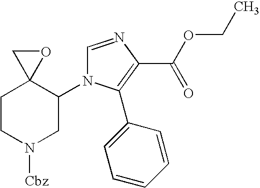

Trimethylsulfoxonium iodide (288 mg) was dissolved in DMSO (5 ml), sodium hydride (60% in oil) (42 mg) was added, and the mixture was stirred at room temperature for 30 min. A solution of benzyl 3-[4-(ethoxycarbonyl)-5-phenyl-1H-imidazol-1-yl]-4-oxopiperidine-1-carboxylate (390 mg) in DMSO (10 ml) was added thereto and the mixture was stirred at room temperature for 30 min. The reaction mixture was poured into a saturated aqueous ammonium chloride solution, and the mixture was extracted with ethyl acetate. The extract was dried over anhydrous sodium sulfate, and the solvent was evaporated in vacuo. The residue was subjected to silica gel column chromatography, and the fraction eluted with ethyl acetate was concentrated in vacuo to give the desired product (298 mg) as an amorphous solid.

¹H-NMR (CDCl₃) δ 1.22 (3H, t), 1.39 (1H, dd), 1.96-2.13 (1H, m), 2.27 (1H, d), 2.61 (1H, d), 3.20 (1H, t), 3.33-3.48 (1H, m), 4.22 (4H, q), 4.31-4.38 (1H, m), 5.10 (2H, s), 7.10-7.59 (10H, m), 7.71 (1H, s)

MS (ESI+, m/e) 462 (M+1)

### Reference Example 352

### Ethyl 3-[(3-morpholinophenyl)amino]-2-nitro-3-phenylacrylate

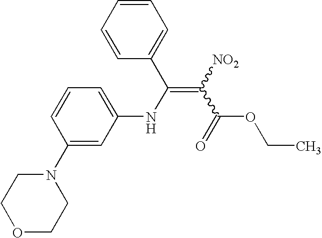

A mixture of ethyl 3-iodo-2-nitro-3-phenylacrylate (950 mg), 3-morpholinoaniline (500 mg), triethylamine (1.2 ml) and acetonitrile (14 ml) was stirred at room temperature for 24 hr, and concentrated in vacuo. The residue was subjected to silica gel column chromatography, and the fraction eluted with ethyl acetate-hexane (1:9 to 1:0) was concentrated in vacuo to give the desired product (855 mg).

¹H-NMR (CDCl₃) δ 0.91 (3H, t), 2.88-2.91 (4H, m), 3.74-3.77 (4H, m), 3.94 (2H, q), 6.24 (1H, br 6.32 (1H, d), 6.61-6.71 (2H, m), 7.04 (1H, t), 7.32-7.44 (8H, m)

MS (ESI+, m/e) 398 (M+1)

### Reference Example 353

### Ethyl 2-methyl-1-(3-morpholinophenyl)-5-phenyl-1H-imidazole-4-carboxylate

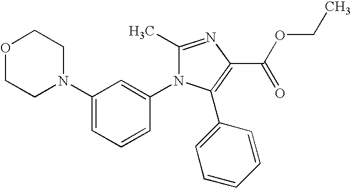

A mixture of ethyl 3-[(3-morpholinophenyl)amino]-2-nitro-3-phenylacrylate (2.0 g), 10% palladium on carbon (containing 50% water) (100 mg) and trimethyl orthoacetate (50 ml) was stirred under hydrogen pressure (5 kgf/cm²) at 80° C. for 14 hr. The catalyst was filtered off, and the filtrate was concentrated in vacuo. The residue was subjected to silica gel column chromatography, and the fraction eluted with ethyl acetate-hexane (1:9 to 1:0) was concentrated in vacuo to give the desired product (1.08 g).

¹H-NMR (CDCl₃) δ 1.28 (3H, t), 2.37 (3H, s), 2.97-3.00 (4H, m), 3.77-3.80 (4H, m), 4.30 (2H, q), 6.43 (1H, dd), 6.60 (1H, dd), 6.84 (1H, dd), 7.19-7.27 (5H, m)

MS (ESI+, m/e) 392 (M+1)

### Reference Example 354

### N-[3-(Methylsulfonyl)phenyl]urea

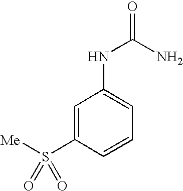

3-(Methylsulfonyl)aniline (1.5 g) was dissolved by heating in a mixed solvent of acetic acid (3 ml) and water (5 ml), and a solution of sodium cyanate (0.94 g) in water (4 ml) was added at 40° C. over 10 min. After stirring at 40° C. for 1 hr, the reaction mixture was diluted with water (30 ml), the crystals were collected by filtration, washed with water and vacuum dried to give the desired product (1.3 g).

¹H-NMR (DMSO-d₆) δ 3.16 (3H, s), 6.02 (2H, s), 7.36-7.54 (2H, m), 7.56-7.66 (1H, m), 8.09 (1H, t), 8.97 (1H, s)

In the same manner as in Reference Example 345, the following compound (Reference Example 355) was obtained.

### Reference Example 355

### N-(3-Morpholinophenyl)urea

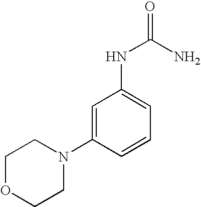

¹H-NMR (DMSO-d₆) δ 2.98-3.09 (4H, m), 3.66-3.77 (4H, m), 5.89 (2H, s), 6.48 (1H, dd), 6.82 (1H, dd), 6.98-7.12 (2H, m), 8.68 (1H, br s)

### Reference Example 356

### Ethyl 1-(2-methoxyphenyl)-2-oxo-5-phenyl-2,3-dihydro-1H-imidazole-4-carboxylate

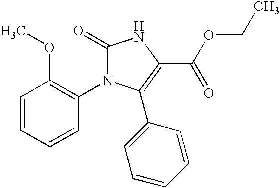

A mixture of 2-methoxyphenylurea (6.00 g), ethyl 2-diazo-3-oxo-3-phenylpropanoate (7.88 g), rhodium(II) acetate diemer (50 mg), toluene (40 ml) and 1,2-dichloroethane (40 ml) was stirred at 80° C. for 5 hr, cooled to room temperature, and concentrated in vacuo. The mixture was diluted with toluene (100 ml), TFA (40 ml) was added, and the reaction mixture was further stirred at 80° C. for 5 hr. The reaction mixture was concentrated in vacuo, and the residue was dissolved in ethyl acetate. Saturated aqueous sodium bicarbonate solution was added by small portions and the mixture was washed with brine, and dried over anhydrous magnesium sulfate. The solvent was evaporated in vacuo, and the crystals were collected by filtration to give the desired product (7.25 g).

¹H-NMR (CDCl₃) δ 1.18 (3H, t), 3.59 (3H, s), 4.18 (2H, q), 6.78-6.83 (1H, m), 6.85-7.01 (2H, ₘ), 7.18-7.30 (6H, m), 8.38 (1H, br s)

MS (ESI+, m/e) 339 (M+1)

In the same manner as in Reference Example 356, the following compounds (Reference Examples 357 to 359) were obtained.

### Reference Example 357

### Ethyl 1-(2-methylphenyl)-2-oxo-5-phenyl-2,3-dihydro-1H-imidazole-4-carboxylate

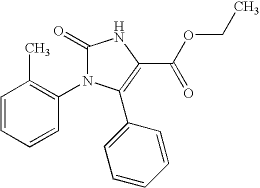

MS (ESI+, m/e) 323 (M+1)

### Reference Example 358

### Ethyl 1-[3-(methylsulfonyl)phenyl]-2-oxo-5-phenyl-2,3-dihydro-1H-imidazole-4-carboxylate

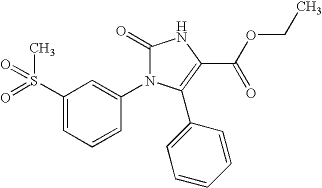

¹H-NMR (DMSO-d₆) δ 1.05 (3H, t), 3.04 (3H, s), 4.08 (3H, q), 7.23-7.82 (9H, m)

### Reference Example 359

### Ethyl 1-(3-morpholinophenyl)-2-oxo-5-phenyl-2,3-dihydro-1H-imidazole-4-carboxylate

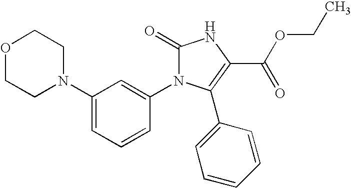

¹H-NMR (CDCl₃) δ 1.18 (3H, t), 2.93-2.99 (4H, m), 3.73-3.81 (4H, m), 4.20 (2H, q), 6.55 (1H, t), 6.64 (1H, dd), 6.76 (1H, dd), 7.10-7.34 (6H, m), 8.28 (1H, s)

### Reference Example 360

### Ethyl 1-(2-nitrophenyl)-2-oxo-5-phenyl-2,3-dihydro-1H-imidazole-4-carboxylate

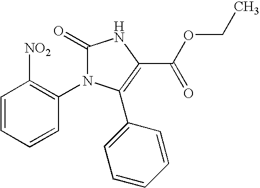

A mixture of 2-nitrophenylurea (2.85 g), ethyl 2-diazo-3-oxo-3-phenylpropanoate (3.12 g), rhodium(II) acetate diemer (32 mg), toluene (100 ml) and 1,2-dichloroethane (100 ml) was stirred at 80° C. for 2 hr, and concentrated in vacuo. The residue was dissolved in TEA (150 ml) and acetic anhydride (50 ml) and the solution was stirred at 75° C. for 12 hr. The reaction mixture was concentrated in vacuo, and the residue was filtrated and washed with methanol to give the desired product (2.60 g).

¹H-NMR (DMSO-d₆) δ 0.93-1.14 (3H, m), 3.97-4.20 (2H, m), 7.18-7.41 (5H, m), 7.49 (1H, d), 7.55-7.80 (2H, m), 7.99-8.18 (1H, m)

MS (ESI+, m/e) 354 (M+1)

### Reference Example 361

### Ethyl 2-ethoxy-1,5-diphenyl-1H-imidazole-4-carboxylate

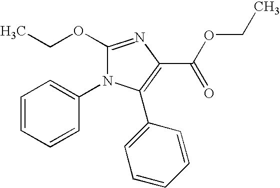

Ethyl 2-oxo-1,5-diphenyl-2,3-dihydro-1H-imidazole-4-carboxylate (500 mg) was dissolved in dichloromethane (10 ml) and the mixture was ice-cooled. Triethyloxonium tetrafluoroborate (1 M dichloromethane solution) (2.5 ml) was added dropwise. The reaction mixture was stirred at room temperature for 3 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:4 to 4:1) was concentrated in vacuo to give the desired product (290 mg).

¹H-NMR (CDCl₃) δ 1.20 (3H, t), 1.35 (3H, t), 4.25 (2H, q), 4.56 (2H, q), 7.04 (1H, dd), 7.06 (1H, s), 7.17-7.31 (8H, m)

MS (ESI+, m/e) 337 (M+1)

In the same manner as in Reference Example 361, the following compounds (Reference Examples 362 to 364) were obtained.

### Reference Example 362

### Ethyl 2-ethoxy-1-(2-methoxyphenyl)-5-phenyl-1H-imidazole-4-carboxylate

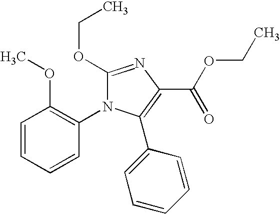

¹H-NMR (CDCl₃) δ 1.17-1.33 (6H, m), 3.63 (3H, s), 4.21-4.35 (2H, m), 4.47-4.61 (2H, m), 6.82-.6.96 (2H, m), 6.99-7.06 (1H, m), 7.14-7.22 (5H, m), 7.25-7.31 (1H, M)

MS (ESI+, m/e) 367 (M+1)

### Reference Example 363

### Ethyl 2-ethoxy-1-[3-(methylsulfonyl)phenyl]-5-phenyl-1H-imidazole-4-carboxylate

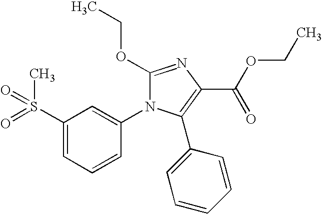

¹H-NMR (CDCl₃) δ 1.20 (3H, t), 2.74-2.79 (3H, m), 4.26 (2H, q), 4.60 (2H, q), 7.15-7.23 (2H, m), 7.25-7.30 (2H, m), 7.41-7.61 (5H, m)

### Reference Example 364

### Ethyl 2-ethoxy-1-(3-morpholophenyl)-5-phenyl-1H-imidazole-4-carboxylate

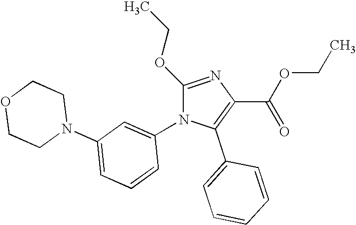

¹H-NMR (CDCl₃) δ 1.21 (3H, t), 1.37 (3H, t), 2.90-2.98 (4H, m), 3.75-3.81 (4H, m), 4.26 (2H, q), 4.57 (2H, q), 6.42-6.46 (1H, m), 6.63 (1H, dd), 6.79 (1H, dd), 7.13-7.31 (6H, m)

### Reference Example 365

### Ethyl 2-methoxy-1,5-diphenyl-1H-imidazole-4-carboxylate

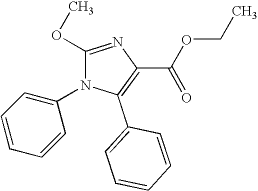

To a solution of ethyl 2-oxo-1,5-diphenyl-2,3-dihydro-1H-imidazole-4-carboxylate (500 mg) and dichloromethane (12 ml) was added trimethyloxonium tetrafluoroborate (800 mg) by small portions. The reaction mixture was stirred at room temperature for 14 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:19 to 7:3) was concentrated in vacuo to give the desired product (316.mg).

¹H-NMR (CDCl₃) δ 1.23 (3H, t), 4.13 (3H, s), 4.26 (2H, q), 7.02-7.09 (2H, m), 7.18-7.32 (8H, m)

MS (ESI+, m/e) 323 (M+1)

In the same manner as in Reference Example 365, the following compounds (Reference Examples 366 to 367) were obtained.

### Reference Example 366

### Ethyl 2-methoxy-1-(2-methylphenyl)-5-phenyl-1H-imidazole-4-carboxylate

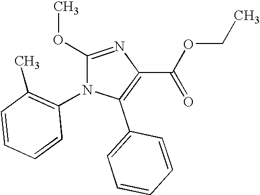

¹H-NMR (CDCl₃) δ 1.24 (3H, t), 2.03 (3H, s), 4.11 (3H, s), 4.29 (2H, q), 6.98-7.03 (1H, m), 7.11-7.26 (8H, m)

MS (ESI+, m/e) 337, (M+1)

### Reference Example 367

### Ethyl 2-methoxy-1-(2-nitrophenyl)-5-phenyl-1H-imidazole-4-carboxylate

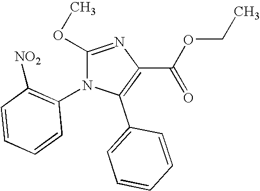

¹H-NMR (CDCl₃) δ 1.22 (3H, t), 4.10 (3H, s), 4.26 (2H, q), 7.03-7.12 (1H, m), 7.19-7.29 (5H, m), 7.44-7.55 (2H, m), 7.95-8.04 (1H, m)

MS (ESI+, m/e) 368 (M+1)

### Reference Example 368

### Ethyl 2-chloro-1,5-diphenyl-1H-imidazole-4-carboxylate

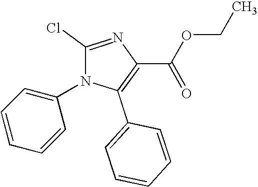

A mixture of ethyl 2-oxo-1,5-diphenyl-2,3-dihydro-1H-imidazole-4-carboxylate (200 mg) and phosphoryl chloride (5.0 ml) was stirred at 100° C. for 18 hr, and cooled to room temperature. The reaction mixture was concentrated in vacuo and and the residue was dissolved in ethyl acetate. The solution was washed successively with saturated aqueous sodium bicarbonate solution and brine, and dried over anhydrous magnesium sulfate. The solvent was evaporated in vacuo to give the desired product (243 mg).

¹H-NMR (CDCl₃) δ 1.27 (3H, t), 4.30 (2H, q), 7.10 (1H, d), 7.12 (1H, d), 7.17-7.30 (5H, m), 7.36 (1H, d), 7.38 (2H, d)

MS (ESI+, m/e) 327 (M+1)

### Reference Example 369

### 1-(1-Benzylpyrrolidin-3-yl)-5-phenyl-1H-imidazole-4-carboxylic acid

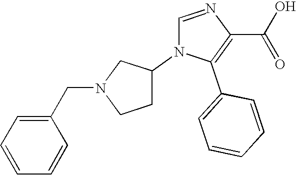

Methyl 1-(1-benzylpyrrolidin-3-yl)-5-phenyl-1H-imidazole-4-carboxylate (8.5 g) was dissolved in a mixed solvent of methanol (40 ml) and water (20 ml), lithium hydroxide monohydrate (3.0 g) was added and the mixture was stirred at 50° C. for 12 hr. The reaction mixture was concentrated in vacuo, the residue was neutralized with 1 N hydrochloric acid, and the mixture was extracted with ethyl acetate-THF (3:1). The extract was dried over anhydrous sodium sulfate, and the solvent was evaporated in vacuo The residue was suspended in ethanol, and concentrated again in vacuo, and the residue was vacuum dried to give the desired product (5.6 g) as an amorphous solid.

MS (ESI+, m/e) 348 (M+1)

### Reference Example 370

### 1-[(1S)-3-Morpholino-3-oxo-1-(2-phenylethyl)propyl]-5-phenyl-1H-imidazole-4-carboxylic acid

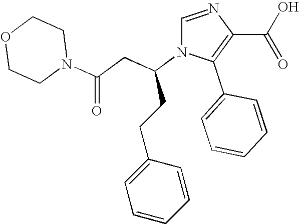

Methyl 1-[(1S)-3-morpholino-3-oxo-1-(2-phenylethyl)propyl]-5-phenyl-1H-imidazole-4-carboxylate (539 mg) was suspended in methanol (20 ml), a 2 N aqueous lithium hydroxide solution (13 ml) was added, and the mixture was stirred at room temperature for 4 hr and at 50° C. for 30 min. The reaction mixture was poured into water, and the mixture was weakly acidified (pH 3) with 6 N hydrochloric acid, and the mixture was extracted with ethyl acetate-THF (3:1). The extract was washed with brine, and dried over anhydrous magnesium sulfate. The solvent was evaporated in vacuo to give the desired product (512 mg), as an amorphous solid.

¹H-NMR (DMSO-d₆) δ 2.00-2.07 (2H, m), 2.31-2.37 (2H, m), 3.03 (1H, dd), 3.19 (1H, dd), 3.25-3.62 (8H, m), 4.29-4.34 (1H, m), 7.03 (2H, d), 7.11-7.24 (4H, m), 7.45-7.47 (5H, m), 8.94 (1H, s)

MS (ESI+, m/e) 434 (M+1)

In the same manner as in Reference Example 370, the following compound (Reference Example 371) was obtained.

### Reference Example 371

### 1-[(1R)-3-Morpholino-3-oxo-1-(2-phenylethyl)propyl]-5-phenyl-1H-imidazole-4-carboxylic acid

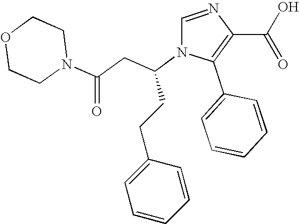

¹H-NMR (DMSO-d₆) δ 1.99-2.07 (2H, m), 2.30-2.35 (2H, m), 3.00 (1H, dd), 3.15 (1H, dd), 3.24-3.62 (8H, m), 4.27-4.31 (1H, m), 7.02 (2H, d), 7.11-7.23 (4H, m), 7.45-7.46 (5H, m), 8.69 (1H, s)

MS (ESI+, m/e) 434 (M+1)

### Reference Example 372

### 1-((2R)-2-{[(Benzyloxy)carbonyl]amino}-2-carboxyethyl)-5-phenyl-1H-imidazole-4-carboxylic acid

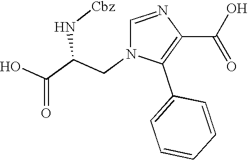

Ethyl 1-((2R)-2-{[(benzyloxy)carbonyl]amino}-3-morpholino-3-oxopropyl)-5-phenyl-1H-imidazole-4-carboxylate (4.07 g) was dissolved in ethanol (90 ml), a 2 N aqueous lithium hydroxide solution (90 ml) was added, and the mixture was stirred at room temperature for 2 hr. The reaction mixture was poured into water, and the mixture was weakly acidified (pH 3) with concentrated hydrochloric acid. The mixture was saturated with sodium chloride, and extracted with ethyl acetate-THF (3:1). The extract was washed with brine, and dried over anhydrous magnesium sulfate. The solvent was evaporated in vacuo, and the crystals were collected by filtration to give the desired product (2.77 g).

¹H-NMR (DMSO-d₆) δ 3.60 (2H, br s), 3.99-4.17 (3H, m), 4.24-4.28 (1H, m), 4.93 (1H, d), 4.98 (1H, d), 7.26-7.39 (6H, m), 7.46-7.48 (3H, m), 7.74 (1H, d), 8.14 (1H, s)

MS (ESI+, m/e) 410 (M+1)

### Reference Example 373

### 1-{(1R)-1-[2-(Methylthio)ethyl]-3-morpholino-3-oxopropyl}-5-phenyl-1H-imidazole-4-carboxylic acid

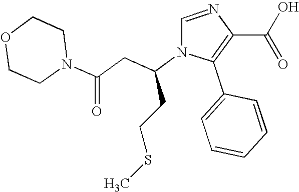

Ethyl 1-{(1R)-1-[2-(methylthio)ethyl]-3-morpholino-3-oxopropyl}-5-phenyl-1H-imidazole-4-carboxylate (292 mg) was dissolved in THF-ethanol (1:1, 35 ml), a 2 N aqueous lithium hydroxide solution (11 ml) was added, and the mixture was stirred at room temperature for 1 hr and at 50° C. for 40 min. The reaction mixture was poured into water, and the mixture was weakly acidified (pH 3) with 6 N hydrochloric acid. The mixture was saturated with sodium chloride, and extracted with ethyl acetate-THF (1:1). The extract was washed with brine, and dried over anhydrous magnesium sulfate. The solvent was evaporated in vacuo to give the desired product (268 mg) as an amorphous solid.

MS (ESI+, m/e) 404 (M+1)

In the same manner as in Reference Example 373, the following compounds (Reference Examples 374 and 375) were obtained.

### Reference Example 374

### 1-[(1S)-3-[(2-Furylmethyl)amino]-1-(2-morpholino-2-oxoethyl)-3-oxopropyl]-5-phenyl-1H-imidazole-4-carboxylic acid

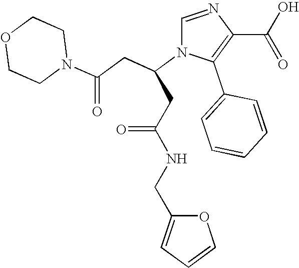

MS (ESI+, m/e) 467 (M+1)

### Reference Example 375

### 1-{2-[(tert-Butoxycarbonyl)(tetrahydro-2H-pyran-4-yl)amino]ethyl}-5-phenyl-1H-imidazole-4-carboxylic acid

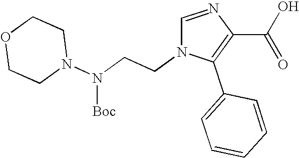

¹H-NMR (DMSO-d₆) δ 1.06-1.10 (2H, m), 1.28-1.42 (13H, m), 3.15 (2H, t), 3.32 (2H, t), 3.74 (2H, dd), 4.00 (2H, t), 7.45-7.55 (5H, m), 9.03 (1H, br s)

MS (ESI+, m/e) 416 (M+1)

### Reference Example 376

### 1-(2,3-Dihydro-1H-inden-2-yl)-5-[3-(3-methoxypropoxy)phenyl]-1H-imidazole-4-carboxylic acid

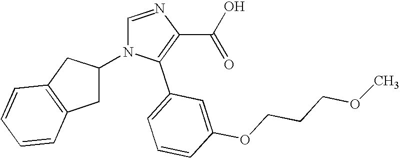

Methyl 1-(2,3-dihydro-1H-inden-2-yl)-5-[3-(3-methoxypropoxy)phenyl]-1H-imidazole-4-carboxylate (2.02 g) was dissolved in methanol (30 ml), a 4 N aqueous sodium hydroxide solution (30 ml) was added, and the mixture was stirred at 50° C. for 50 min. The reaction mixture was poured into water, and the mixture was weakly acidified (pH 3) with concentrated hydrochloric acid. The mixture was saturated with sodium chloride, and extracted with ethyl acetate-THF (3:1). The extract was washed with brine, and dried over anhydrous magnesium sulfate. The solvent was evaporated in vacuo, and the crystals were collected by filtration to give the desired product (1.58 g).

¹H-NMR (DMSO-d₆) δ 1.95 (2H, quintet), 3.22-3.25 (7H, m), 3.47 (2H, t), 4.04 (2H, t), 4.66 (1H, quintet), 6.96-7.04 (3H, m), 7.17-7.25 (4H, m), 7.38 (1H, t), 7.84 (1H, s)

MS (ESI+, m/e) 393 (M+1)

In the same manner as in Reference Example 376, the following compound (Reference Example 377) was obtained.

### Reference Example 377

### 5-Phenyl-1-(4-hydroxytetrahydro-2H-pyran-3-yl)-1H-imidazole-4-carboxylic acid

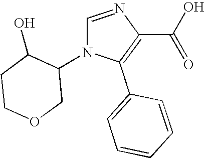

¹H-NMR (CDCl₃) δ 1.15-1.35 (2H, m), 2.31 (1H, br s), 2.55-2.88 (2H, m), 3.04-4.05 (4H, m), 7.21-7.40 (5H, m), 7.43-7.72 (1H, m)

### Reference Example 378

### 5-Phenyl-1-(1,2,3,4-tetrahydronaphthalen-2-yl)-1H-imidazole-4-carboxylic acid

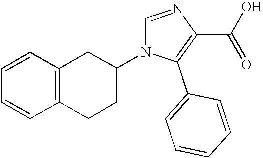

Methyl 5-phenyl-1-(1,2,3,4-tetrahydronaphthalen-2-yl)-1H-imidazole-4-carboxylate (800 mg) was dissolved in THF-methanol (1:1, 10 ml), a 8 N aqueous sodium hydroxide solution (1 ml) was added, and the mixture was stirred at 50° C. for 3 hr. The reaction mixture was concentrated in vacuo, and the residue was partitioned between ethyl acetate and 1 N hydrochloric acid. The organic layer was washed with brine, and dried over anhydrous sodium sulfate, and the solvent was evaporated in vacuo to give the desired product (766 mg) as an amorphous solid.

¹H-NMR (CDCl₃) δ 2.09-2.23 (2H, m), 2.82-2.95 (2H, m), 3.12 (2H, d), 4.20-4.36 (1H, m), 7.02-7.18 (4H, m), 7.37-7.50 (5H, m), 7.70 (1H, s)

MS (ESI+, m/e) 319 (M+1)

In the same manner as in Reference Example 378, the following compounds (Reference Examples 379 to 382) were obtained.

### Reference Example 379

### 1-(2-Hydroxy-2-phenylethyl)-5-phenyl-1H-imidazole-4-carboxylic acid

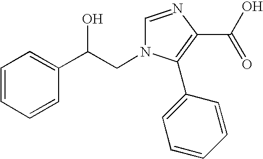

¹H-NMR (DMSO-d₆) δ 3.89 (2H, d), 4.53 (1H, t), 5.75 (1H, br s), 6.97-7.06 (2H, m), 7.20-7.32 (5H, m), 7.44 (3H, s), 7.76 (1H, s), 11.87 (1H, br s)

MS (ESI+, m/e) 309 (M+1)

### Reference Example 380

### 1-(2-Oxoazepan-3-yl)-5-phenyl-1H-imidazole-4-carboxylic acid

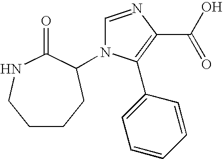

¹H-NMR (DMSO-d₆) δ 1.14-1.29 (1H, m), 1.40-1.80 (2H, m), 1.81-2.29 (3H, m), 2.63-2.78 (1H, m), 3.00 (1H, s), 4.54 (1H, d), 7.14-7.53 (5H, m), 7.81 (1H, s), 7.93 (1H, t), 11.44 (1H, br s)

MS (ESI+, m/e) 300 (M+1)

### Reference Example 381

### 1-(2-Oxotetrahydrofuran-5-yl)-5-phenyl-1H-imidazole-4-carboxylic acid

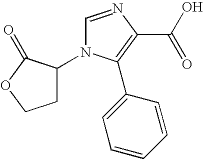

¹H-NMR (DMSO-d₆) δ 2.62-2.75 (2H, m), 4.27-4.41 (2H, m), 5.03 (1H, t), 7.35-7.50 (5H, m), 8.07 (1H, s), 11.98 (1H, br s)

MS (ESI+, m/e) 273 (M+1)

### Reference Example 382

### 1-[trans-4-Hydroxy-1-(phenylsulfonyl)piperidin-3-yl]-5-phenyl-1H-imidazole-4-carboxylic acid

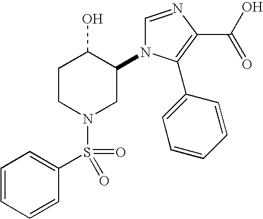

¹H-NMR (DMSO-d₆) δ 1.19-1.33 (1H, m), 1.91 (1H, s), 2.74 (1H, t), 3.13-3.73 (3H, m), 3.95 (1H, s), 5.35 (1H, br s), 7.29-7.44 (3H, m), 7.49 (3H, s), 7.54-7.76 (5H, m), 7.98 (1H, s), 12.70 (1H, br s)

MS (ESI+, m/e) 428 (M+1)

### Reference Example 383

### 1-{1-[(Benzyloxy)carbonyl]piperidin-3-yl-5-phenyl-1H-imidazole-4-carboxylic acid

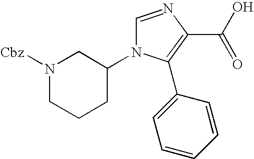

Methyl 1-(1-[(benzyloxy)carbonyl]piperidin-3-yl}-5-phenyl-1H-imidazole-4-carboxylate (6.65 g) was dissolved in THF-methanol (1:1, 60 ml), a 8 N aqueous sodium hydroxide solution (5 ml) was added, and the mixture was stirred at 50° C. for 3 hr. The reaction mixture was concentrated in vacuo, and the residue was dissolved in water (30 ml), and the solution was neutralized with 6 N hydrochloric acid under ice-cooling. The precipitated crystals were collected by filtration and vacuum dried to give the desired product (5.67 g).

¹H-NMR (CDCl₃) δ 1.45 (1H, s), 1.81 (2H, s), 2.10 (1H, s), 2.79 (1H, s), 2.95 (1H, t, J=11.7), 3.81 (1H, br s), 4.09 (1H, br s), 4.24 (1H, br s), 4.95-5.10 (2H, m), 6.56 (1H, br s), 7.26-7.39 (10H, m), 7.78 (1H, s)

MS (ESI+, m/e) 406 (M+1)

### Reference Example 384

### 1-(2-Hydroxy-2-pyridin-2-ylethyl)-5-phenyl-1H-imidazole-4-carboxylic acid

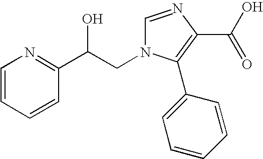

Methyl 1-(2-hydroxy-2-pyridin-2-ylethyl)-5-phenyl-1H-imidazole-4-carboxylate (323 mg) was dissolved in THF-methanol (1:1, 10 ml), a 8 N aqueous sodium hydroxide solution (1 ml) was added, and the mixture was stirred at 50° C. for 3 hr. The reaction mixture was concentrated in vacuo, the residual aqueous solution was neutralized with 1 N hydrochloric acid, and the mixture was extracted with ethyl acetate. The extract was washed with brine, and dried over anhydrous sodium sulfate, and the solvent was evaporated in vacuo to give the desired product (264 mg) as an amorphous solid.

¹H-NMR (DMSO-d₆) δ 3.35 (1H, br s), 3.99 (1H, br s), 4.21 (1H, d), 4.63 (1H, br s), 6.11 (1H, br s), 7.14-7.51 (8H, m), 7.72 (1H, s), 8.37 (1H, d)

MS (ESI+, m/e) 310 (M+1)

### Reference Example 385

### 2-Ethoxy-1,5-diphenyl-1H-imidazole-4-carboxylic acid

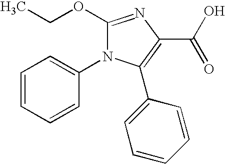

A mixture of ethyl 2-ethoxy-1,5-diphenyl-1H-imidazole-4-carboxylate (290 mg), a 8 N aqueous sodium hydroxide solution (2 ml), ethanol (7 ml) and water (2 ml) was stirred at 70° C. for 15 hr, and cooled to 0° C. The reaction mixture was acidified (pH 1) with 1 N hydrochloric acid, and the mixture was extracted with ethyl acetate. The extract was washed successively with water and brine, and dried over anhydrous magnesium sulfate. The solvent was evaporated in vacuo to give the desired product (259 mg).

¹H-NMR (CDCl₃) δ 1.38 (3H, t), 4.51 (2H, d), 7.06 (2H, dd), 7.21-7.34 (8H, m)

MS (ESI+, m/e) 309 (M+1)

In the same manner as in Reference Example 385, the following compounds (Reference Examples 386 to 388) were obtained.

### Reference Example 386

### 2-Ethoxy-1-(2-methoxyphenyl)-5-phenyl-1H-imidazole-4-carboxylic acid

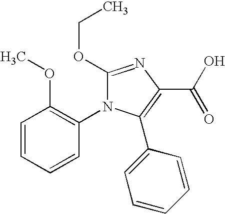

¹H-NMR (DMSO-d₆) δ 1.24 (3H, t), 3.59 (3H, s), 4.35 (2H, q), 6.83-6.97 (1H, m), 6.99-7.08 (1H, m), 7.12-7.25 (6H, m), 7.26-7.38 (2H, m)

MS (ESI+, m/e) 339 (M+1)

### Reference Example 387

### 2-Methoxy-1-(2-methylphenyl)-5-phenyl-1H-imidazole-4-carboxylic acid

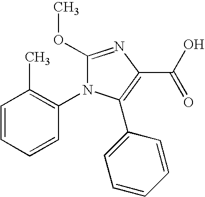

¹H-NMR (DMSO-d₆) δ 1.94 (3H, s), 3.92 (3H, br s), 7.14-7.27 (9H, m)

MS (ESI+, m/e) 309 (M+1)

### Reference Example 388

### 2-Methoxy-1-(2-nitrophenyl)-5-phenyl-1H-imidazole-4-carboxylic acid

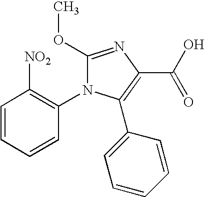

¹H-NMR (CDCl₃) δ 4.08 (3H, s), 7.07-7.14 (1H, m), 7.21-7.31 (5H, m), 7.49-7.58 (2H, m), 8.01-8.07 (1H, m)

MS (ESI+, m/e) 340 (M+1)

### Reference Example 389

### 2-Methoxy-1,5-diphenyl-1H-imidazole-4-carboxylic acid

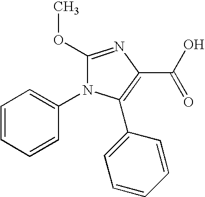

A mixture of ethyl 2-methoxy-1,5-diphenyl-1H-imidazole-4-carboxylate (311 mg), a 8 N aqueous sodium hydroxide solution (1 ml), ethanol (6 ml) and water (1 ml) was stirred at 80° C. for 12 hr, and cooled to 0° C. The reaction mixture was acidified (pH 1) with 1 N hydrochloric acid, and the resulting precipitate was filtrated and washed with water to give the desired product (241 mg).

¹H-NMR (DMSO-d₆) δ 3.98 (3H, s), 7.15-7.24 (7H, m), 7.29-7.37 (3H, m), 11.96 (1H, br s)

MS (ESI+, m/e) 295 (M+1)

### Reference Example 390

### 5-Cyclohexyl-1-phenyl-1H-imidazole-4-carboxylic acid

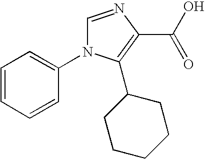

Methyl 5-cyclohexyl-1-phenyl-1H-imidazole-4-carboxylate (130 mg) was dissolved in THF-methanol (1:1, 10 ml), a 8 N aqueous sodium hydroxide solution (2 ml) was added, and the mixture was stirred at 50° C. for 3 hr. The reaction mixture was concentrated in vacuo, neutralized with 1 N hydrochloric acid, subjected to Diaion HP-20 (manufactured by Mitsubishi Chemical), and washed with water. The fraction eluted with acetone was concentrated in vacuo to give the desired product (676 mg) as an amorphous solid.

¹H-NMR (DMSO-d₆) δ 1.09 (3H, s), 1.40-1.75 (5H, m), 1.77-2.05 (2H, m), 2.73-2.85 (1H, m), 7.35-7.49 (2H, m), 7.52-7.70 (3H, m), 7.86 (1H, s)

MS (ESI+, m/e) 271 (M+1)

In the same manner as in Reference Example 390, the following compound (Reference Example 391) was obtained.

### Reference Example 391

### 5-Cyclopropyl-1-phenyl-1H-imidazole-4-carboxylic acid

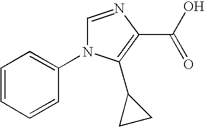

¹H-NMR (CDCl₃) δ 0.36-0.49 (2H, m), 0.63-0.80 (2H, m), 1.91-2.01 (1H, m), 3.52 (1H, br s), 7.49-7.60 (5H, m), 7.89 (1H, s)

MS (ESI+, m/e) 229 (M+1)

### Reference Example 392

### Ethyl N-(tert-butoxycarbonyl)-4-fluoro-D-phenylalanyl-N-benzylglycinate

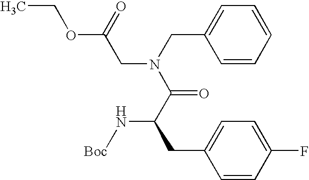

A solution of N-(tert-butoxycarbonyl)-4-fluoro-D-phenylalanine (5.00 g), ethyl N-benzylglycinate (3.41 g), WSC.HCl (4.06 g), HOBt (2.62 g) and DMF (90 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo, and the crystals were collected by filtration to give the desired product (6.87 g).

MS (ESI+, m/e) 359 (M+1−“Boc”)

In the same manner as in Reference Example 392, the following compounds (Reference Examples 393 and 394) were obtained.

### Reference Example 393

### Ethyl N-(tert-butoxycarbonyl)-D-leucyl-N-benzylglycinate

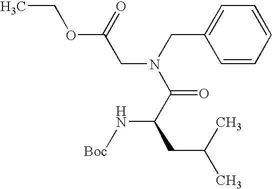

MS (ESI+, m/e) 307 (M+1−“Boc”)

### Reference Example 394

### Ethyl N-(tert-butoxycarbonyl)-3-cyclohexyl-D-alanyl-N-benzylglycinate

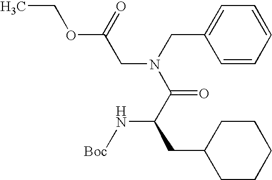

¹H-NMR (CDCl₃) δ 0.74-1.88 (25H, m), 3.70-3.89 (1H, m), 4.09-4.29 (2H, m), 4.42-4.61 (2H, m), 4.74-4.92 (2H, m), 5.10-5.18 (1H, m), 7.18-7.38 (5H, m)

### Reference Example 395

### (3R)-1-Benzyl-3-(4-fluorobenzyl)piperazine-2,5-dione

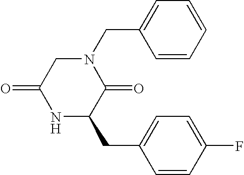

To a solution of ethyl N-(tert-butoxycarbonyl)-4-fluoro-D-phenylalanyl-N-benzylglycinate (6.86 g) in dichloromethane (4 ml) was added TFA (40 ml), and the mixture was stirred at room temperature for 1 hr. The reaction mixture was concentrated in vacuo, the residue was diluted with toluene and the solution was concentrated again in vacuo to remove TFA. The residue was dissolved in dichloromethane (60 ml), triethylamine (12 ml) was added and the mixture was stirred at room temperature for 2 hr. The reaction mixture was concentrated in vacuo, and the residue was dissolved in ethyl acetate-THF (3:1, 200 ml). The solution was washed successively with a 10% aqueous citric acid solution, water, saturated aqueous sodium bicarbonate solution, water and brine, and dried over anhydrous magnesium sulfate. The solvent was evaporated in vacuo, and the crystals were collected by filtration to give the desired product (4.26 g).

¹H-NMR (CDCl₃) δ 3.02 (1H, d), 3.08 (1H, dd), 3.21 (1H, dd), 3.55 (1H, d), 4.32-4.36 (1H, m), 4.39 (1H, d), 4.55 (1H, d), 6.68 (1H, s), 6.84-6.91 (2H, m), 7.07-7.18 (4H, m), 7.30-7.33 (3H, m)

MS (ESI+, m/e) 313 (M+1)

In the same manner as in Reference Example 395, the following compounds (Reference Examples 396 and 397) were obtained.

### Reference Example 396

### (3R)-1-Benzyl-3-isobutylpiperazine-2,5-dione

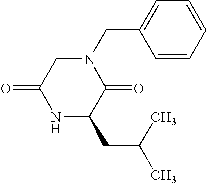

MS (ESI+, m/e) 261 (M+1)

### Reference Example 397

### (3R)-1-Benzyl-3-(cyclohexylmethyl)piperazine-2,5-dione

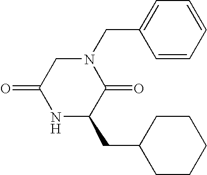

¹H-NMR (CDCl₃) δ 0.93-1.05 (2H, m), 1.12-1.29 (3H, m), 1.40-1.46 (1H, m), 1.57-1.89 (8H, m), 3.76-3.89 (2H, m), 4.06-4.12 (1H, m), 4.59 (2H, dd), 6.98 (1H, s), 7.24-7.38 (5H, m)

### Reference Example 398

### (3R)-1-Benzyl-3-(4-fluorobenzyl)piperazine

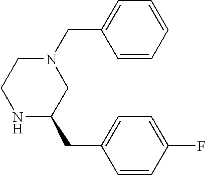

A mixture of (3R)-1-benzyl-3-(4-fluorobenzyl)piperazine-2,5-dione (4.25 g) and THF (120 ml) was ice-cooled, lithium aluminum hydride (2.07 g) was added by small portions. The mixture was stirred at room temperature for 30 min and at 60° C. for 15 hr, and cooled to −78° C., and ethanol-ethyl acetate (1:1, 14 ml) and a 1 N aqueous sodium hydroxide solution (28 ml) were successively added dropwise. After the completion of the dropwise addition, the mixture was stirred at room temperature for 40 min, the insoluble material was filtered off, and washed with ethyl acetate. The filtrate was washed with brine, and dried over anhydrous magnesium sulfate. The solvent was evaporated in vacuo. The residue was subjected to basic silica gel column chromatography, and the fraction eluted with ethyl acetate-hexane (1:2 to 1:1) was concentrated in vacuo, and the crystals were collected by filtration to give the desired product (2.50 g).

¹H-NMR (CDCl₃) δ 1.62 (1H, br s), 1.86 (1H, t), 2.03-2.11 (1H, m), 2.51(1H, dd), 2.64-2.99 (6H, m), 3.46 (1H, d), 3.53 (1H, d), 6.49-7.00 (2H, m), 7.12-7.18 (2H, m), 7.21-7.32 (5H, m)

MS (ESI+, m/e) 285 (M+1)

In the same manner as in Reference Example 398, the following compounds (Reference Examples 399 and 400) were obtained.

### Reference Example 399

### (3R)-1-Benzyl-3-isobutylpiperazine

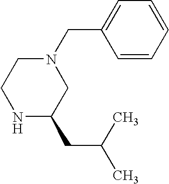

MS (ESI+, m/e) 233 (M+1)

### Reference Example 400

### (3R)-1-Benzyl-3-(cyclohexylmethyl)piperazine

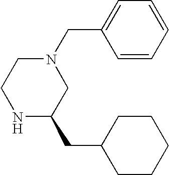

MS (ESI+, m/e) 273 (M+1)

### Reference Example 401

### tert-Butyl (2S)-4-benzyl-2-(hydroxymethyl)piperazine-1-carboxylate

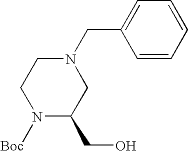

[(2S)-4-Benzylpiperazin-2-yl]methanol (25.84 g) was dissolved in THF (250 ml), di-tert-butyl dicarbonate (27.34 g) was added by small portions, and the mixture was stirred at room temperature for 2 hr. The reaction mixture, was concentrated in vacuo, the residue was subjected to silica gel column chromatography, and the fraction eluted with ethyl acetate-hexane (1.5:1) was concentrated in vacuo to give the desired product (38.34 g) as an oil.

¹H-NMR (CDCl₃) δ 1.45 (9H, s), 2.09 (1H, dt), 2.31 (1H, dd), 2.83 (1H, d), 2.97 (1H, d), 3.36-3.53 (3H, m), 3.83-3.99 (5H, m), 7.25-7.33 (5H, m)

MS (ESI+, m/e) 307 (M+1)

In the same manner as in Reference Example 401, the following compound (Reference Example 402) was obtained.

### Reference Example 402

### tert-Butyl (2R)-4-benzyl-2-(2-hydroxyethyl)piperazine-1-carboxylate

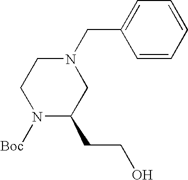

¹H-NMR (CDCl₃) δ 1.46 (9H, s), 2.01 (1H, dt), 2.20-2.24 (1H, m), 2.25 (1H, dd), 2.68-2.72 (2H, m), 3.01 (1H, dt), 3.37-3.60 (4H, m), 3.85-3.98 (3H, m), 4.26-4.30 (1H, m), 7.25-7.34 (5H, m)

MS (ESI+, m/e) 321 (M+1)

### Reference Example 403

### tert-Butyl (2S)-4-benzyl-2-formylpiperazine-1-carboxylate

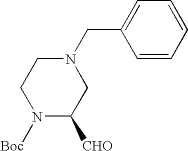

tert-Butyl (2S)-4-benzyl-2-(hydroxymethyl)piperazine-1-carboxylate (12.26 g) was dissolved in dichloromethane (130 ml), a solution of pyridine.sulfur trioxide complex (19.10 g) in DMSO (130 ml) and triethylamine (12.14 g) were added at 0° C. The reaction mixture was stirred at 0° C. for 2 hr, and the mixture was poured into ice-cooled saturated aqueous sodium bicarbonate solution, and the mixture was extracted with ethyl acetate. The, extract was washed successively with water and brine, and dried over anhydrous magnesium sulfate. The solvent was evaporated in vacuo. The residue was subjected to silica gel column chromatography, and the fraction eluted with ethyl acetate-hexane (1:4) was concentrated in vacuo to give the desired product (6.28 g) as an oil.

¹H-NMR (CDCl₃) δ 1.43-1.48 (9H, m), 2.12 (1H, dt), 2.27 (1H, dd), 2.69-2.73 (1H, m), 3.06-3.15 (1H, m), 3.30 (1H, d), 3.44 (1H, d), 3.56 (1H, d), 3.78 (0.5H, d), 3.90 (0.5H, d), 4.38 (0.5H, s), 4.58 (0.5H, s), 7.22-7.34 (5H, m), 9.49 (1H, s)

MS (ESI+, m/e) 305 (M+1)

In the same manner as in Reference Example 403, the following compounds (Reference Examples 404) were obtained.

### Reference Example 404

### tert-Butyl (2R)-4-benzyl-2-(2-oxoethyl)piperazine-1-carboxylate

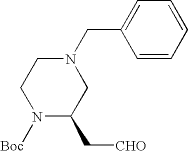

¹H-NMR (CDCl₃) δ 1.44 (9H, s), 2.04 (1H, dt), 2.20 (1H, dd), 2.66-2.84 (4H, m), 3.01-3.09 (1H, m), 3.42 (1H, d), 3.51 (1H, d), 3.84-3.88 (1H, m), 4.60-4.64 (1H, m), 7.25-7.28 (5H, m), 9.73 (1H, s)

MS (ESI+, m/e) 319 (M+1)

### Reference Example 405

### Di-tert-butyl (2R)-2-(4-hydroxybenzyl)piperazine-1,4-dicarboxylate

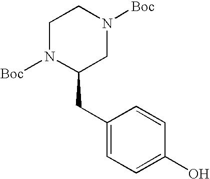

4-{[(2R)-4-Benzylpiperazin-2-yl]methyl}phenol (12 g) was dissolved in methanol (240 ml), 20% palladium hydroxide on carbon (containing 50% water) (3.0 g) was added, and a catalytic hydrogenation was performed at room temperature and atmospheric pressure for 12 hr. The catalyst was filtered off, and the filtrate was concentrated in vacuo. The residue was suspended in ethyl acetate, the solution was dried over anhydrous sodium sulfate, and the solvent was evaporated in vacuo. The residue was dissolved in a mixed solvent of tert-butanol (100 ml) and water (100 ml), and 2.5 N sodium hydroxide (40 ml) and di-tert-butyl dicarbonate (17.6 g) were added under ice-cooling. After stirring for 12 hr, the mixture was extracted with ethyl acetate. The extract was washed successively with a 10% aqueous citric acid solution and brine, and dried over anhydrous sodium sulfate. The solvent was evaporated in vacuo. The residue was subjected to silica gel column chromatography, and the fraction eluted with ethyl acetate-hexane (1:4) was concentrated in vacuo to give the desired product (10.7 g) as an amorphous solid.

MS (ESI+, m/e) 393 (M+1)

### Reference Example 406

### Di-tert-butyl (2R)-2-(4-{[(trifluoromethyl)sulfonyl]oxy}benzyl)piperazine-1,4-dicarboxylate

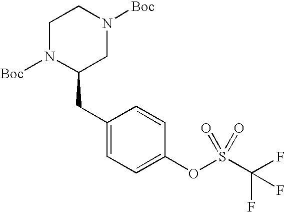

Di-tert-butyl (2R)-2-(4-hydroxybenzyl)piperazine-1,4-dicarboxylate (10.7 g), 4-nitrophenyl trifluoromethanesulfonate (8.1 g) and potassium carbonate (7.6 g) were suspended in DMF (170 ml), and the mixture was stirred at room temperature for 12 hr. The reaction mixture was poured into water and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (11.2 g) as an amorphous solid.

MS (ESI+, m/e) 525 (M+1)

### Reference Example 407

### Di-tert-butyl (2R)-2-[4-(ethoxycarbonyl)benzyl]piperazine-1,4-dicarboxylate

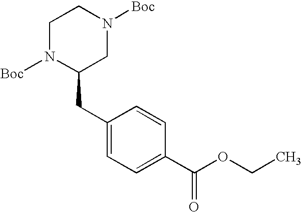

Di-tert-butyl (2R)-2-(4-{[(trifluoromethyl)sulfonyl]oxy}benzyl)piperazine-1,4-dicarboxylate (6.0 g), triethylamine (11 ml), palladium(II) acetate (510 mg) and dppf (1.26 g) were suspended in ethanol (65 ml), and the mixture was stirred under carbon monoxide atmosphere at 80° C. for 12 hr. The reaction mixture was cooled to room temperature, and diluted with ethyl acetate and water, and the insoluble material was filtered off using Celite. The organic layer was separated, washed with brine, and dried over magnesium sulfate. The solvent was evaporated in vacuo. The residue was subjected to silica gel column chromatography, and the fraction eluted with ethyl acetate-hexane (1:4) was concentrated in vacuo, and the crystals were collected by filtration to give the desired product (4.1 g).

MS (ESI+, m/e) 449 (M+1)

### Reference Example 408

### 4-{[(2R)-1,4-Bis(tert-butoxycarbonyl)piperazin-2-yl]methyl}benzoic acid

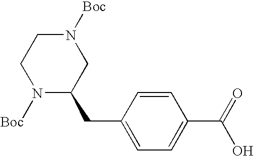

Di-tert-butyl (2R)-2-[4-(ethoxycarbonyl)benzyl]piperazine-1,4-dicarboxylate (1.26 g) was dissolved in ethanol (30 ml), potassium hydroxide (788 mg) was added, and the mixture was heated under reflux for 5 hr. The solvent was evaporated in vacuo, and the residue was adjusted to pH 5 with 1 N hydrochloric acid. The liberated oil was extracted with ethyl acetate, and the extract was washed with brine, and dried over anhydrous magnesium sulfate. The solvent was evaporated in vacuo to give the desired product (1.17 g) as an oil.

NMR (CDCl₃) δ 1.39 (9H, s), 1.53 (9H, s), 2.69-2.96 (4H, m), 3.04-3.18 (2H, m), 3.83-4.14 (2H, m), 4.20-4.35 (1H, m), 7.22-7.40 (2H, m), 8.01-8.07 (2H, m)

### Reference Example 409

### Di-tert-butyl (2R)-2-[4-(hydroxymethyl)benzyl]piperazine-1,4-dicarboxylate

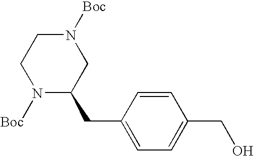

4-{[(2R)-1,4-Bis(tert-butoxycarbonyl)piperazin-2-yl]methyl}benzoic acid (1.17 g) and 4-methylmorpholine (0.367 ml) were dissolved in THF (20 ml), and the solution was cooled to 0° C. Ethyl chloroformate (0.391 ml) was added thereto and the mixture was stirred at the same temperature for 1 hr. Sodium borohydride (319 mg) and methanol (1 ml) were added to the reaction mixture, and the mixture was stirred at 0° C. for 1 hr and at room temperature for 1 hr. Aqueous sodium bicarbonate solution was added to the reaction mixture, and the mixture was extracted with ethyl acetate. The extract was washed with brine, dried over anhydrous magnesium sulfate, and concentrated in vacuo. The residue was subjected to silica gel column chromatography, and the fraction eluted with hexane-ethyl acetate (1:1) was concentrated in vacuo to give the desired product (888 mg) as an oil.

NMR (CDCl₃) δ 1.40 (9H, s), 1.50 (9H, s), 2.62-2.95 (4H, m), 3.03-3.20 (1H, m), 3.78-4.36 (4H, m), 4.66 (2H, s), 7.14-7.38 (4H, m)

### Reference Example 410

### tert-Butyl (2S)-4-benzyl-2-(1-hydroxy-2-methyl-propyl)piperazine-1-carboxylate

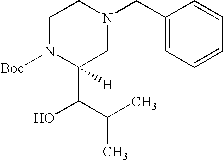

tert-Butyl (2S)-4-benzyl-2-formylpiperazine-1-carboxylate (2.5 mg) was dissolved in THF (25 ml) and the mixture was cooled to −78° C. Isopropylmagnesium bromide (1 M THF solution, 6.2 ml) was added thereto and the mixture was stirred at the same temperature for 30 min. A saturated aqueous ammonium chloride solution was added to the reaction mixture, and the mixture was extracted with ethyl acetate. The extract was washed with brine, and dried over anhydrous sodium sulfate, and the solvent was evaporated in vacuo. The residue was subjected to basic silica gel column chromatography, and the fraction eluted with ethyl acetate was concentrated in vacuo to give the desired product (2.7 g) as an oil.

MS (ESI+, m/e) 349 (M+1)

### Reference Example 411

### tert-Butyl (2S)-4-benzyl-2-(1-hydroxy-2-methylpropyl)piperazine-1-carboxylate

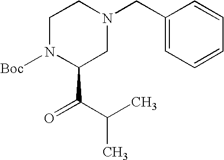

To a solution of tert-butyl (2S)-4-benzyl-2-(1-hydroxy-2-methylpropyl)piperazine-1-carboxylate (2.0 g) in dichloromethane (20 ml) was added a solution of Dess-Martin reagent (2.9 g) in dichloromethane (30 ml) and the mixture was stirred at room temperature for hr. The reaction mixture was concentrated in vacuo. The residue was subjected to silica gel column chromatography, and the fraction eluted with ethyl acetate-hexane (3:1) was concentrated in vacuo to give the desired product (1.4 g) as an amorphous solid.

MS (ESI+, m/e) 347 (M+1)

### Reference Example 412

### tert-Butyl (2S)-4-benzyl-2-[cyclopropyl(hydroxy)methyl]piperazine-1-carboxylate

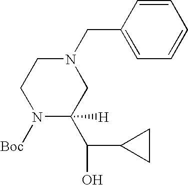

tert-Butyl (2S)-4-benzyl-2-formylpiperazine-1-carboxylate (2.5 g) was dissolved in THF (25 ml), and the mixture was cooled to −30° C. Cyclopropylmagnesium bromide (0.5 M THF solution, 40 ml) was added thereto and the mixture was stirred at −20° C. for 1 hr. A saturated aqueous ammonium chloride solution was added to the reaction mixture, and the mixture was extracted with ethyl acetate. The extract was washed with brine, and dried over anhydrous sodium sulfate, and concentrated in vacuo. The residue was subjected to basic silica gel column chromatography, and the fraction eluted with ethyl acetate was concentrated in vacuo to give the desired product (2.2 g) as an amorphous solid.

MS (ESI+, m/e) 347 (M+1)

In the same manner as in Reference Example 412, the following compounds (Reference Examples 413 and 414) were obtained.

### Reference Example 413

### tert-Butyl (2R)-4-benzyl-2-(2-cyclopropyl-2-hydroxyethyl)piperazine-1-carboxylate

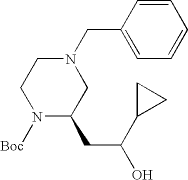

MS (ESI+, m/e) 361 (M+1)

### Reference Example 414

### tert-Butyl (2S)-4-benzyl-2-[(4-fluorophenyl)(hydroxy)methyl]piperazine-1-carboxylate

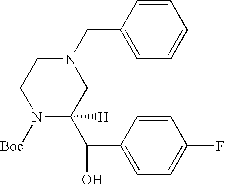

MS (ESI+, m/e) 401 (M+1)

In the same manner as in Reference Example 412, the following compound (Reference Example 415) was obtained by reacting methyl(1,4-dibenzylpiperazin-2-yl)acetate with methylmagnesium bromide.

### Reference Example 415

### 1-(1,4-Dibenzylpiperazin-2-yl)-2-methylpropan-2-ol

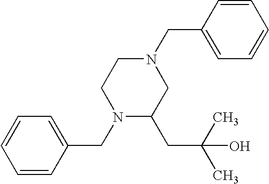

MS (ESI+, m/e) 339 (M+1)

### Reference Example 416

### tert-Butyl (2S)-4-benzyl-2-{[(2-methylprop-2-en-1-yl)oxy]methyl}piperazine-1-carboxylate

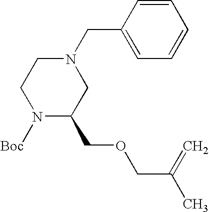

tert-Butyl (2S)-4-benzyl-2-(hydroxymethyl)piperazine-1-carboxylate (500 mg) and 3-bromo-2-methyl-1-propene (446 mg) were dissolved in DMF (5 ml), sodium hydride (60% in oil) (130 mg) was added under ice-cooling and the mixture was stirred at room temperature for 1 hr and at 60° C. for 1 hr. The reaction mixture was poured into ice-water (20 ml), and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (730 mg) as an amorphous solid.

MS (ESI+, m/e) 361 (M+1)

### Reference Example 417

### tert-Butyl (2R)-2-[(E)-2-cyclopropylethenyl]-4-benzylpiperazine-1-carboxylate

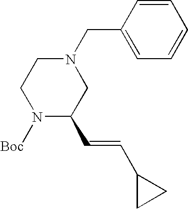

(Cyclopropylmethyl)(triphenyl)phosphonium bromide (385 mg) was dissolved in THF (10 ml) and the mixture was cooled to −78° C. n-Butyllithium (1.6M hexane solution) (1.25 ml) was added and the mixture was stirred at −20° C. for 20 min. A solution of tert-butyl (2S)-2-formyl-4-benzylpiperazine-1-carboxylate (608 mg) in THF (5 ml) was added, and the mixture was stirred at −20° C. for 2 hr. A saturated aqueous ammonium chloride solution was added to the reaction mixture, and the mixture was extracted with ethyl acetate. The extract was dried over anhydrous sodium sulfate, and the solvent was evaporated in vacuo. The residue was subjected to silica gel column chromatography, and the fraction eluted with ethyl acetate-hexane (1:4) was concentrated in vacuo to give the desired product (700 mg) as an oil.

MS (ESI+, m/e) 343 (M+1)

### Reference Example 418

### tert-Butyl (2R)-4-benzyl-2-[(E)-2-(Pyridin-2-yl)vinyl]piperazine-1-carboxylate

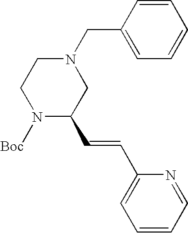

tert-Butyl (2S)-4-benzyl-2-formylpiperazine-1-carboxylate (500 mg) was dissolved in THF (5 ml) and the mixture was cooled to 0° C. Triphenyl(pyridin-2-ylmethyl)phosphonium chloride.potassium hydride (1:1) (1059 mg) was added thereto, and the mixture was stirred at room temperature for 17 hr. Brine was added to the reaction mixture, and the mixture was extracted with ethyl acetate, dried over anhydrous magnesium sulfate, and concentrated in vacuo. The residue was subjected to basic silica gel column chromatography, and the fraction eluted with ethyl acetate-hexane (1:1) was concentrated in vacuo to give the desired product (590 mg) as an oil.

MS (ESI+, m/e) 380 (M+1)

### Reference Example 419

### tert-Butyl (2R,6S)-2,4-dibenzyl-6-(hydroxymethyl)piperazine-1-carboxylate

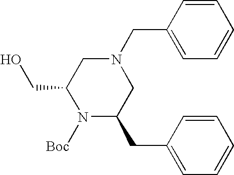

tert-Butyl (2R)-2,4-dibenzylpiperazine-1-carboxylate (1.0 g) and TMEDA (2.25 ml) were dissolved in THF (30 ml), and the mixture was cooled to −78° C. sec-Butyllithium (1 M hexane solution, 9 ml) was added thereto over 15 min, and the mixture was stirred at −50° C. for 15 min. DMF (660 mg) was added thereto and the mixture was stirred at −50° C. for 10 min, and at room temperature for 30 min. A saturated aqueous ammonium chloride solution was added to the reaction mixture, and the mixture was extracted with ethyl acetate. The extract was washed with brine, dried over anhydrous sodium sulfate, and concentrated in vacuo. The residue was subjected to silica gel column chromatography, and the fraction eluted with ethyl acetate-hexane (2:1) was, concentrated in vacuo to give tert-butyl (2R,6S)-2,4-dibenzyl-6-formylpiperazine-1-carboxylate (1.0 g) as an amorphous solid. A 900 mg portion thereof was dissolved in methanol (90 ml), and sodium borohydride (431 mg) was added under ice-cooling. After stirring at 0° C. for 1 hr, ice-water (5 ml) was added. The solvent was evaporated in vacuo, and the suspension was extracted with ethyl acetate. The extract was washed with brine, and dried over anhydrous sodium sulfate, and the solvent was evaporated in vacuo. The residue was subjected to basic silica gel column chromatography, and the fraction eluted with ethyl acetate-hexane solution (1:2) was concentrated in vacuo to give the desired product (730 mg) as an amorphous solid.

MS (ESI+, m/e) 397 (M+1)

### Reference Example 420

### tert-Butyl (2R,6R)-2,4-dibenzyl-6-methylpiperazine-1-carboxylate

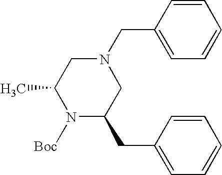

tert-Butyl (2R)-2,4-dibenzylpiperazine-1-carboxylate (1.0 g) and TMEDA (2.25 ml) were dissolved in THF (30 ml) and the mixture was cooled to −78° C. sec-Butyllithium (1 M hexane solution, 9 ml) was added thereto over 15 min, and the mixture was stirred at −50° C. for 15 min. Methyl iodide (1.28 g) was added thereto and the mixture was stirred at −50° C. for 10 min, and at room temperature for 30 min. A saturated aqueous ammonium chloride solution was added to the reaction mixture, and the mixture was extracted with ethyl acetate. The extract was washed with brine, dried over anhydrous sodium sulfate, and concentrated in vacuo. The residue was subjected to silica gel column chromatography, and the fraction eluted with ethyl acetate-hexane (2:1) was concentrated in vacuo to give the desired product (790 mg) as an oil.

MS (ESI+, m/e) 397 (M+1)

### Reference Example 421

### tert-Butyl (2R)-4-benzyl-2-{4-[2-(benzyloxy)-2-oxoethoxy]benzyl}piperazine-1-carboxylate

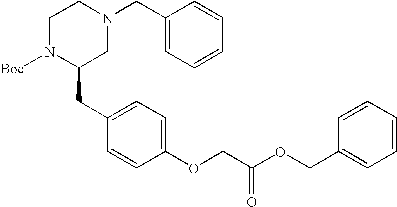

To a solution of tert-butyl (2R)-4-benzyl-2-(4-hydroxybenzyl)piperazine-1-carboxylate (1.5 g), benzyl bromoacetate (1.0 g) and DMF (15 ml) was added potassium carbonate (813 mg). After stirring at 80° C. for 2 hr, the mixture was poured into ice-water, and the mixture was extracted with ethyl acetate. The extract was dried over anhydrous sodium sulfate, and the solvent was evaporated in vacuo. The residue was subjected to silica gel column chromatography, and the fraction eluted with ethyl acetate-methanol (4:1) was concentrated in vacuo to give the desired product (1.7 g) as crystals.

MS (ESI+, m/e) 531 (M+1)

### Reference Example 422

### tert-Butyl (2R)-4-benzyl-2-(4-cyanobenzyl)piperazine-1-carboxylate

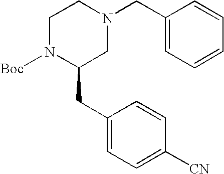

A solution of tert-butyl (2R)-4-benzyl-2-(4-{[(trifluoromethyl)sulfonyl]oxy)benzyl)piperazine-1-carboxylate (3.6 g), zinc cyanide (1 g), tetrakis(triphenylphosphine)palladium(0) (810 mg) and DMF (30 ml) was stirred at 80° C. for 15 hr. The insoluble material was filtered off, and the solvent was evaporated in vacuo. The residue was subjected to silica gel column chromatography, and the fraction eluted with ethyl acetate-hexane (1:9 to 3:7) was concentrated in vacuo to give the desired product (1.25 g) as crystals.

MS (ESI+, m/e) 392 (M+1)

### Reference Example 423

### tert-Butyl (2R)-4-benzyl-2-[(isopropylamino)methyl]piperazine-1-carboxylate

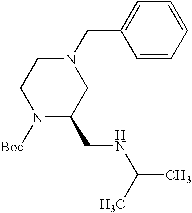

A solution of tert-butyl (2S)-4-benzyl-2-formylpiperazine-1-carboxylate (6.27 g), isopropylamine (2.44 g), acetic acid (2.47 g), dichloromethane (80 ml) and DMF (40 ml) was stirred at room temperature for 40 min, sodium triacetoxyborohydride (8.73 g) was added and the mixture was stirred at room temperature for additional 15 hr. The reaction mixture was poured into saturated aqueous sodium bicarbonate solution, and the mixture was stirred at room temperature for 15 min, and extracted with ethyl acetate. The extract was washed successively with water and brine, and dried over anhydrous magnesium sulfate. The solvent was evaporated in vacuo to give the desired product (6.37 g) as an oil.

¹H-NMR (CDCl₃) δ 0.98 (3H, d), 1.00 (3H, d), 1.46 (9H, s), 1.99-2.08 (2H, m), 2.73-2.96 (6H, m), 3.07 (1H, dt), 3.38 (1H, d), 3.54 (1H, d), 3.85-3.89 (1H, m), 4.07 (1H, br s), 7.30-7.32 (5H, m)

MS (ESI+, m/e) 348 (M+1)

In the same manner as in Reference Example 423, the following compounds (Reference Examples 424 and 425) were obtained.

### Reference Example 424

### tert-Butyl (2R)-2-(anilinomethyl)-4-benzylpiperazine-1-carboxylate

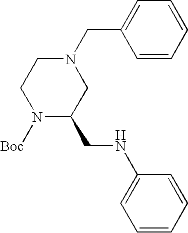

¹H-NMR (CDCl₃) δ 1.45 (9H, s), 2.02-2.11 (2H, m), 2.80-2.84 (2H, m), 3.12 (1H, dt), 3.39-4.28 (7H, m), 6.54 (2H, d), 6.62-6.67 (1H, m), 7.10-7.15 (2H, m), 7.27-7.34 (5H, m)

MS (ESI+, m/e) 382 (M+1)

### Reference Example 425

### tert-Butyl (2R)-4-benzyl-2-{[(2,4-dimethoxybenzyl)amino]methyl}piperazine-1-carboxylate

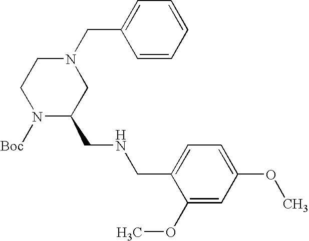

¹H-NMR (CDCl₃) δ 1.44 (9H, s), 1.59 (1H, br s), 1.97 (1H, dd), 2.00 (1H, dd), 2.09 (1H, dd), 2.71 (1H, d), 2.85-3.03 (4H, m), 3.46 (2H, s), 3.71 (2H, s), 3.77 (3H, s), 3.80 (3H, s), 3.80-3.86 (1H, m), 6.40-6.46 (2H, m), 7.12 (1H, d), 7.20-7.33 (5H, m)

MS (ESI+, m/e) 456 (M+1)

### Reference Example 426

### tert-Butyl (2S)-4-benzyl-2-{[(4-ethoxy-4-oxobutanoyl)(isopropyl)amino]methyl}piperazine-1-carboxylate

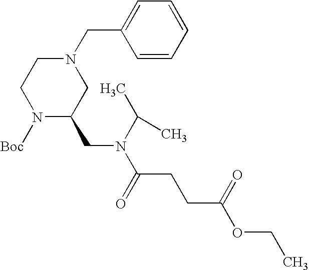

tert-Butyl (2R)-4-benzyl-2-[(isopropylamino)methyl]piperazine-1-carboxylate (2.12 g) and triethylamine (1.23 g) were dissolved in THF (50 ml), ethylsuccinyl chloride (2.01 g) was added. After stirring at room temperature for 15 hr, the mixture was poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:2 to 1:1) was concentrated in vacuo to give the desired product (2.89 g) as an oil.

MS (ESI+, m/e) 476 (M+1)

In the same manner as in Reference Example 426, the following compounds (Reference Examples 427 to 430) were obtained.

### Reference Example 427

### tert-Butyl (2S)-4-benzyl-2-{[(4-ethoxy-4-oxobutanoyl)(phenyl)amino]methyl}piperazine-1-carboxylate

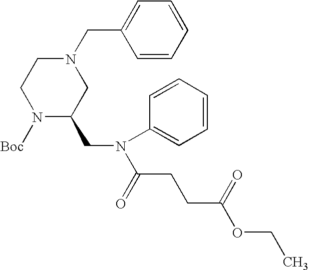

MS (ESI+, m/e) 510 (M+1)

### Reference Example 428

### tert-Butyl (2S)-4-benzyl-2-{[(2,4-dimethoxybenzyl)(2-methoxybenzoyl)amino]methyl}piperazine-1-carboxylate

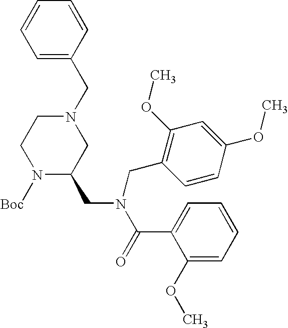

MS (ESI+, m/e) 590 (M+1)

### Reference Example 429

### tert-Butyl (2S)-2-{[(benzoyl)(2,4-dimethoxybenzyl)amino]methyl}-4-benzylpiperazine-1-carboxylate

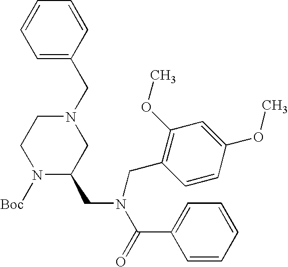

MS (ESI+, m/e) 560 (M+1)

### Reference Example 430

### tert-Butyl (2S)-4-benzyl-2-{[(cyclohexylcarbonyl)(2,4-dimethoxybenzyl)amino]methyl}piperazine-1-carboxylate

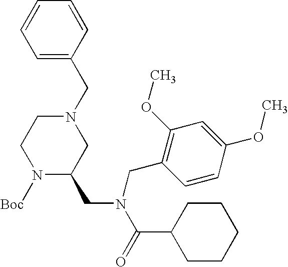

MS (ESI+, m/e) 566 (M+1)

### Reference Example 431

### 4-[{[(2S)-4-Benzyl-1-(tert-butoxycarbonyl)piperazin-2-yl]methyl}(isopropyl)amino]-4-oxobutyric acid

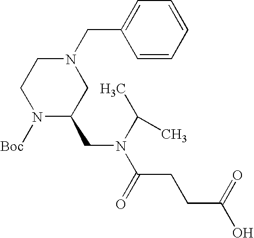

tert-Butyl (2S)-4-benzyl-2-{[(4-ethoxy-4-oxobutanoyl)(isopropyl)amino]methyl}piperazine-1-carboxylate (2.88 g) was dissolved in ethanol (100 ml), and a 2 N aqueous lithium hydroxide solution (65 ml) was added. After stirring at room temperature for 1 hr, the mixture was poured into ice-water. The mixture was neutralized by adding 6 N hydrochloric acid by small portions with vigorous stirring. The mixture was saturated with sodium chloride, and extracted with ethyl acetate. The extract was washed with brine, and dried over anhydrous magnesium sulfate. The solvent was evaporated in vacuo to give the desired product (2.29 g) as an amorphous solid.

MS (ESI+, m/e) 448 (M+1)

In the same manner as in Reference Example 431, the following compound (Reference Example 432) was obtained.

### Reference Example 432

### 4-[{[(2S)-4-Benzyl-1-(tert-butoxycarbonyl)piperazin-2-yl]methyl}(phenyl)amino]-4-oxobutyric acid

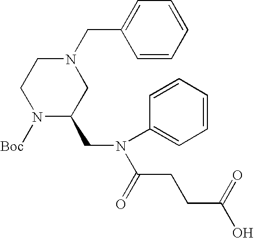

MS (ESI+, m/e) 482 (M+1)

### Reference Example 433

### tert-Butyl (2S)-2-{[(4-amino-4-oxobutanoyl)(isopropyl)amino]methyl}-4-benzylpiperazine-1-carboxylate

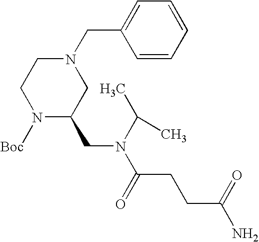

A mixture of 4-[{[(2S)-4-benzyl-1-(tert-butoxycarbonyl)piperazin-2-yl]methyl}(isopropyl)amino]-4-oxobutyric acid (2.28 g), HOBt ammonium salt (930 mg), WSC.HCl (1.17 g) and DMF (35 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo, and the crystals were collected by filtration to give the desired product (2.12 g).

MS (ESI+, m/e) 447 (M+1)

In the same manner as in Reference Example 433, the following compound (Reference Example 434) was obtained.

### Reference Example 434

### tert-Butyl (2S)-2-{[(4-amino-4-oxobutanoyl)(phenyl)amino]methyl}-4-benzylpiperazine-1-carboxylate

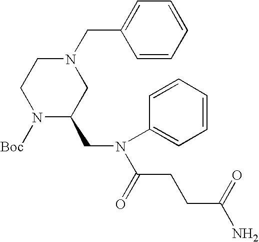

MS (ESI+, m/e) 481 (M+1)

### Reference Example 435

### tert-Butyl (2S)-4-benzyl-2-{[[4-(cyclopropylamino)-4-oxobutanoyl](isopropyl)amino]methyl}piperazine-1-carboxylate

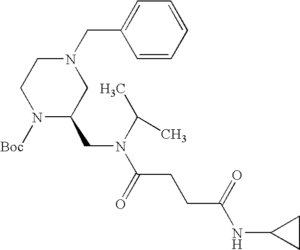

A solution of 4-[{[(2S)-4-benzyl-1-(tert-butoxycarbonyl)piperazin-2-yl]methyl}(isopropyl)amino]-4-oxobutyric acid (1.96 g), cyclopropylamine (275 mg), WSC.HCl (1.01 g), HOBt (710 mg) and DMF (25 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane (1:1 to 1:0) was concentrated in vacuo to give the desired product (1.80 g) as an amorphous solid.

MS (ESI+, m/e) 487 (M+1)

In the same manner as in Reference Example 435, the following compounds (Reference Examples 436 to 438) were obtained.

### Reference Example 436

### tert-Butyl (2S)-4-benzyl-2-{[isopropyl(4-morpholino-4-oxobutanoyl)amino]methyl}piperazine-1-carboxylate

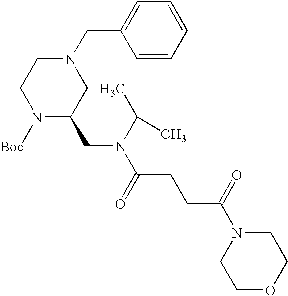

MS (ESI+, m/e) 517 (M+1)

### Reference Example 437

### tert-Butyl (2S)-4-benzyl-2-{[[4-(cyclopropylamino)-4-oxobutanoyl](phenyl)amino]methyl}piperazine-1-carboxylate

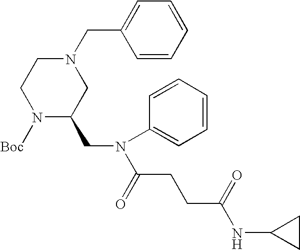

MS (ESI+, m/e) 521 (M+1)

### Reference Example 438

### tert-Butyl (2S)-4-benzyl-2-{[(4-morpholino-4-oxobutanoyl)(phenyl)amino]methyl}piperazine-1-carboxylate

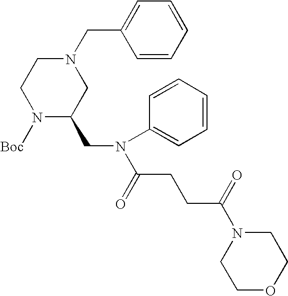

MS (ESI+, m/e) 551 (M+1)

### Reference Example 439

### tert-Butyl (2S)-4-benzyl-2-{[isopropyl(5-methoxy-4,4-dimethyl-5-oxopentanoyl)amino]methyl}piperazine-1-carboxylate

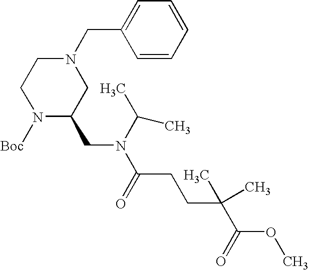

5-Methoxy-4,4-dimethyl-5-oxovaleric acid (4.46 g) was dissolved in THF (100 ml), oxalyl chloride (3.90 g) and DMF (50 μl) were added. After stirring at room temperature for 2 hr, the reaction mixture was concentrated in vacuo, and the residue was dissolved in THF (10 ml). The solution was added to a solution of tert-butyl (2R)-4-benzyl-2-[(isopropylamino)methyl]piperazine-1-carboxylate (4.24 g) and triethylamine (2.59 g) in THF (90 ml). After stirring at room temperature for 15 hr, the reaction mixture was poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:3 to 1:1) was concentrated in vacuo to give the desired product (5.91 g) as an oil.

MS (ESI+, m/e) 504 (M+1)

In the same manner as in Reference Example 439, the following compound (Reference Example 440) was obtained.

### Reference Example 440

### tert-Butyl (2S)-4-benzyl-2-{[(5-methoxy-4,4-dimethyl-5-oxopentanoyl)(phenyl)amino]methyl)piperazine-1-carboxylate

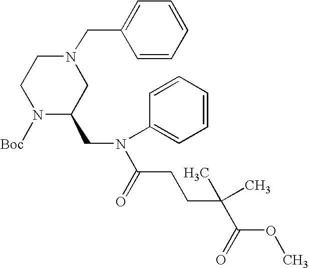

MS (ESI+, m/e) 538 (M+1)

### Reference Example 441

### tert-Butyl (2R)-2-(2-hydroxyethyl)piperazine-1-carboxylate

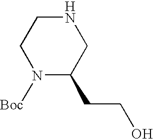

tert-Butyl (2R)-4-benzyl-2-(2-hydroxyethyl)piperazine-1-carboxylate (13.33 g) was dissolved in methanol (135 ml), 20% palladium hydroxide on carbon (containing 50% water) (4.0 g) was added, and a catalytic hydrogenation was performed under a pressure of 5.0 kgf/cm² at room temperature for 4 hr. The catalyst was filtered off, and the filtrate was concentrated in vacuo to give the desired product (9.44 g) as an oil.

¹H-NMR (CDCl₃) δ 1.47 (9H, s), 1.68 (1H, br s), 2.07-2.11 (1H, m), 2.36-2.40 (3H, m), 2.64-2.75 (1H, m), 2.85-2.96 (3H, m), 3.38-3.42 (1H, m), 3.66 (1H, dt), 3.82-3.86 (1H, m), 4.24 (1H, br s)

MS (ESI+, m/e) 231 (M+1)

### Reference Example 442

### 4-Benzyl 1-tert-butyl (2R)-2-(2-hydroxyethyl)piperazine-1,4-dicarboxylate

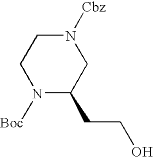

tert-Butyl (2R)-2-(2-hydroxyethyl)piperazine-1-carboxylate (9.44 g) was dissolved in dioxane (90 ml) and the mixture was ice-cooled. A solution of sodium carbonate (4.78 g) in water (45 ml) and benzyl chloroformate (7.34 g) were added. After stirring at room temperature for 2 hr, the reaction mixture was poured into water, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:1 to 2:1) was concentrated in vacuo to give the desired product (14.17 g) as an oil.

MS (ESI+, m/e) 265 (M+1−“Boc”)

### Reference Example 443

### 4-Benzyl 1-tert-butyl (2R)-2-{2-[(methylsulfonyl)oxy]ethyl}piperazine-1,4-dicarboxylate

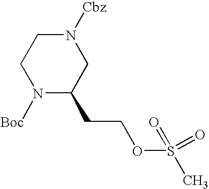

4-Benzyl 1-tert-butyl (2R)-2-(2-hydroxyethyl)piperazine-1,4-dicarboxylate (14.17 g) and triethylamine (5.90 g) were dissolved in THF (80 ml), and the mixture was ice-cooled and methanesulfonyl chloride (5.57 g) was added thereto. After stirring at room temperature for 2 hr, the reaction mixture was poured into water, and the mixture was extracted with ethyl acetate. The extract was washed successively with water and brine, and dried over anhydrous magnesium sulfate. The solvent was evaporated in vacuo, and the crystals were collected by filtration to give the desired product (15.54 g).

¹H-NMR (CDCl₃) δ 1.47 (9H, s), 1.88-2.04 (2H, m), 2.93-2.98 (5H, m), 3.95-4.33 (7H, m), 5.10 (1H, d), 5.17 (1H, d), 7.30-7.39 (5H, m)

MS (ESI+, m/e) 343 (M+1−“Boc”)

### Reference Example 444

### 4-Benzyl 1-tert-butyl (2R)-2-(2-phenoxyethyl)piperazine-1,4-dicarboxylate

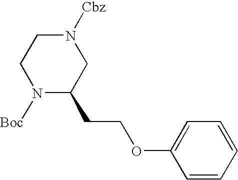

A mixture of 4-benzyl 1-tert-butyl (2R)-2-{2-[(methylsulfonyl)oxy]ethyl}piperazine-1,4-dicarboxylate (708 mg), phenol (188 mg), potassium carbonate (332 mg), potassium iodide (133 mg) and DMF (16 ml) was stirred at 65° C. for 15 hr, and poured into water, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane (1:2) was concentrated in vacuo to give the desired product (591 mg) as an oil.

MS (ESI+, m/e) 441 (M+1)

### Reference Example 445

### Benzyl (3R)-3-(2-phenoxyethyl)piperazine-1-carboxylate

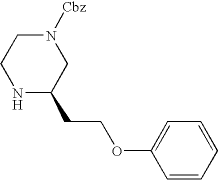

4-Benzyl 1-tert-butyl (2R)-2-(2-phenoxyethyl)piperazine-1,4-dicarboxylate (585 mg) was dissolved in dichloromethane (2 ml), TFA (4 ml) was added and the mixture was stirred at room temperature for 50 min. The reaction mixture was poured into saturated aqueous sodium bicarbonate solution-brine (1:1) by small portions, and the mixture was basified by adding potassium carbonate by small portions, and extracted with ethyl acetate. The extract was washed with brine, and dried over anhydrous magnesium sulfate. The solvent was evaporated in vacuo to give the desired product (435 mg) as an oil.

MS (ESI+, m/e) 341 (M+1)

### Reference Example 446

### tert-Butyl (3R)-3-[4-(hydroxymethyl)benzyl]piperazine-1-carboxylate

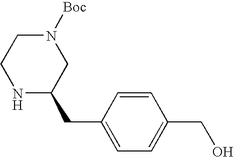

Di-tert-butyl (2R)-2-[4-(hydroxymethyl)benzyl]piperazine-1,4-dicarboxylate (880 mg) was dissolved in chloroform (5 ml), TFA (5 ml) was added, and the mixture was stirred at room temperature for 15 hr. The reaction mixture was concentrated in vacuo, toluene was added to the residue and the mixture was concentrated in vacuo. The residue was dissolved in THF (15 ml), N,N-diisopropylethylamine (1.5 ml) was added and the mixture was cooled to 0° C. di-tert-Butyl dicarbonate (452 mg) was added to the reaction mixture and the mixture was stirred at the same temperature for 1 hr and at room temperature for 2 hr. Aqueous sodium bicarbonate solution was added to the reaction mixture, and the mixture was extracted with ethyl acetate. The extract was washed with brine, dried over anhydrous magnesium sulfate, and concentrated in vacuo to give the desired product (830 mg) as an oil.

MS (ESI+, m/e) 307 (M+1)

### Reference Example 447

### 4-{[(2R)-4-Benzylpiperazin-2-yl]methyl}benzonitrile

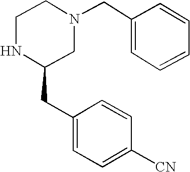

tert-Butyl (2R)-4-benzyl-2-(4-cyanobenzyl)piperazine-1-carboxylate (1.2 g) was dissolved in dichloromethane (1 ml), TFA (3 ml) was added, and the mixture was stirred at room temperature for 1 hr and concentrated in vacuo. The residue was neutralized with 6% aqueous sodium bicarbonate solution, and the mixture was extracted with chloroform. The extract was dried over anhydrous magnesium sulfate and the solvent was evaporated in vacuo to give the desired product (820 mg) as an oil.

MS (ESI+, m/e) 292 (M+1)

### Reference Example 448

### [(2S)-4-Benzylpiperazin-2-yl](4-fluorophenyl)methanol

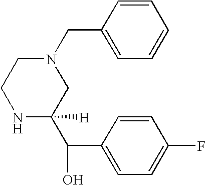

tert-Butyl (2S)-4-benzyl-2-[(4-fluorophenyl)(hydroxy)methyl]piperazine-1-carboxylate (552 mg) was dissolved in chloroform (5 ml), and TFA (5 ml) was added. After stirring at room temperature for 1 hr, the reaction mixture was concentrated in vacuo, and the residue was diluted with small portions of aqueous, sodium bicarbonate solution. The mixture was saturated with sodium chloride, and extracted with ethyl acetate. The extract was dried over anhydrous sodium sulfate, and the solvent was evaporated in vacuo, and the crystals were collected by filtration to give the desired product (340 mg).

MS (ESI+, m/e) 301 (M+1)

### Reference Example 449

### 1-[(2S)-4-Benzylpiperazin-2-yl]-2-methylpropan-1-ol

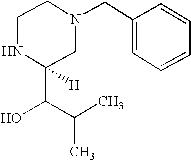

tert-Butyl (2S)-4-benzyl-2-(1-hydroxy-2-methylpropyl)piperazine-1-carboxylate (1.4 g) was dissolved in chloroform (20 ml), and TFA (10 ml) was added. After stirring at room temperature for 1 hr, the solvent was evaporated in vacuo. The residue was subjected to silica gel column chromatography, and the fraction eluted with ethyl acetate-methanol (1:1) was concentrated in vacuo to give the desired product (1.1 g) as an oil.

MS (ESI+, m/e) 249 (M+1)

In the same manner as in Reference Example 449, the following compounds (Reference Examples 450 to 457) were obtained.

### Reference Example 450

### 1-[(2S)-4-Benzylpiperazin-2-yl]-2-methylpropan-1-one

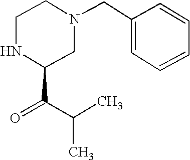

MS (ESI+, m/e) 247 (M+1)

### Reference Example 451

### [(2S)-4-Benzylpiperazin-2-yl](cyclopropyl)methanol

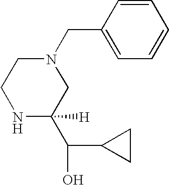

MS (ESI+, m/e) 247 (M+1)

### Reference Example 452

### 2-[(2R)-4-Benzylpiperazin-2-yl]-1-cyclopropylethanol

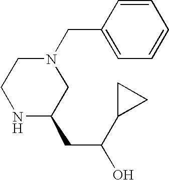

MS (ESI+, m/e) 261 (M+1)

### Reference Example 453

### (3S)-1-Benzyl-3-{[(2-methylprop-2-en-1-yl)oxy]methyl}piperazine

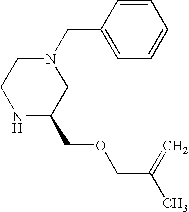

MS (ESI+, m/e) 261 (M+1)

### Reference Example 454

### (3R)-3-[(E)-2-Cyclopropylethenyl]-1-benzylpiperazine

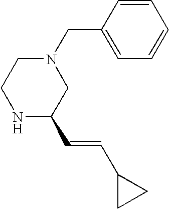

MS (ESI+, m/e) 243 (M+1)

### Reference Example 455

### [(2S,6R)-4,6-Dibenzylpiperazin-2-yl]methanol

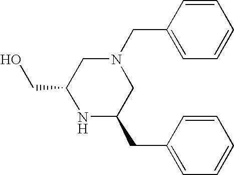

MS (ESI+, m/e) 297 (M+1)

### Reference Example 456

### (3R,5R)-1,3-Dibenzyl-5-methylpiperazine

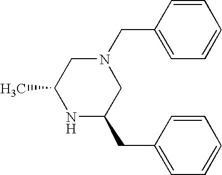

MS (ESI+, m/e) 281 (M+1)

### Reference Example 457

### Benzyl (4-{[(2R)-4-benzylpiperazin-2-yl]methyl}phenoxy)acetate

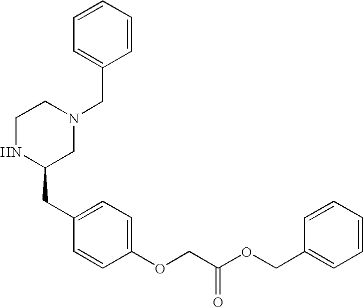

MS (ESI+, m/e) 431 (M+1)

### Reference Example 458

### N-{[(2S)-4-Benzylpiperazin-2-yl]methyl}-N-isopropylsuccinamide

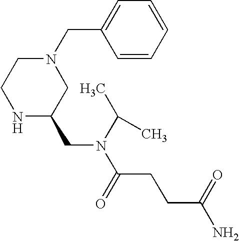

tert-Butyl (2S)-2-{[(4-amino-4-oxobutanoyl)(isopropyl)amino]methyl)-4-benzylpiperazine-1-carboxylate (2.11 g) was dissolved in dichloromethane (6 ml), TFA (12 ml) was added and the mixture was stirred at room temperature for 50 min. The reaction mixture was poured into saturated aqueous sodium bicarbonate solution by small portions, and basified with a 1 N aqueous sodium hydroxide solution. The mixture was saturated with sodium chloride, and extracted with chloroform. The extract was dried over anhydrous magnesium sulfate, and the solvent was evaporated in vacuo to give the desired product (1.63 g) as an oil.

MS (ESI+, m/e) 347 (M+1)

In the same manner as in Reference Example 458, the following compounds (Reference Examples 459 to 465) were obtained.

### Reference Example 459

### N-{[(2S)-4-Benzylpiperazin-2-yl]methyl}-N-phenylsuccinamide

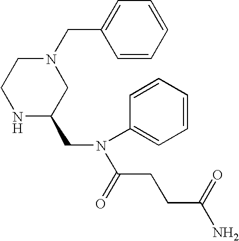

MS (ESI+, m/e) 381 (M+1)

### Reference Example 460

### N-{[(2S)-4-Benzylpiperazin-2-yl]methyl}-N′-cyclopropyl-N-isopropylsuccinamide

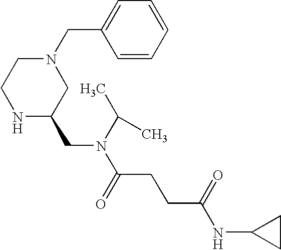

MS (ESI+, m/e) 387 (M+1)

### Reference Example 461

### N-{[(2S)-4-Benzylpiperazin-2-yl]methyl}-N-isopropyl-4-morpholino-4-oxobutanamide

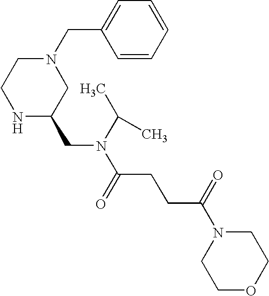

MS (ESI+, m/e) 417 (M+1)

### Reference Example 462

### N-{[(2S)-4-Benzylpiperazin-2-yl]methyl}-N′-cyclopropyl-N-phenylsuccinamide

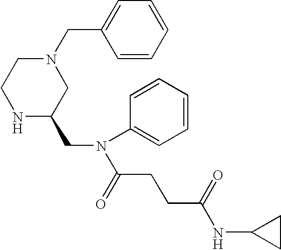

MS (ESI+, m/e) 421 (M+1)

### Reference Example 463

### N-{[(2S)-4-benzylpiperazin-2-yl]methyl}-4-morpholino-4-oxo-N-phenylbutanamide

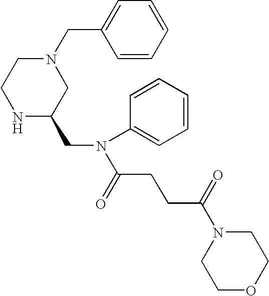

MS (ESI+, m/e) 451 (M+1)

### Reference Example 464

### Methyl 5-[{[(2S)-4-benzylpiperazin-2-yl]methyl}(isopropyl)amino]-2,2-dimethyl-5-oxovalerate

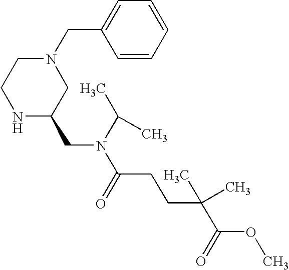

MS (ESI+, m/e) 404 (M+1)

### Reference Example 465

### Methyl 5-[{[(2S)-4-benzylpiperazin-2-yl]methyl}(phenyl)amino]-2,2-dimethyl-5-oxovalerate

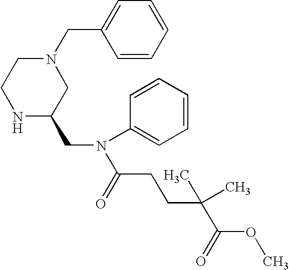

MS (ESI+, m/e) 438 (M+1)

### Reference Example 466

### N-{[(2R)-4-Benzylpiperazin-2-yl]methyl}-2-methoxybenzamide

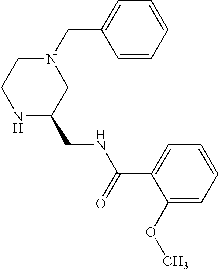

tert-Butyl (2S)-4-benzyl-2-{[(2,4-dimethoxybenzyl)(2-methoxybenzoyl)amino]methyl}piperazine-1-carboxylate (1.89 g) was dissolved in dichloromethane (3 ml), TFA (12 ml) was added and the mixture was stirred at room temperature for 1.5 hr. The reaction mixture was poured into saturated aqueous sodium bicarbonate solution by small portions, and the mixture was basified by adding potassium carbonate by small portions, and the mixture was extracted with ethyl acetate (during which the insoluble material was filtered off). The extract was washed with brine, and dried over anhydrous magnesium sulfate. The solvent was evaporated in vacuo, and the mixture was concentrated to about 50 ml, the insoluble material was filtered off again. The,filtrate was concentrated in vacuo, and the crystals were collected by filtration to give the desired product (1.09 g).

¹H-NMR (CDCl₃) δ 2.01 (1H, t), 2.22 (1H, dt), 2.78 (1H, d), 2.88 (1H, d), 2.96 (1H, dt), 3.12 (1H, dt), 3.19-3.27 (1H, m), 3.44-3.57 (4H, m), 3.85-3.96 (4H, m), 6.94 (1H, d), 7.05 (1H, dt), 7.22-7.32 (5H, m), 7.43 (1H, ddd), 8.13 (1H, dd), 8.18 (1H, t)

MS (ESI+, m/e) 340 (M+1)

In the same manner as in Reference Example 466, the following compound (Reference Example 467) was obtained.

### Reference Example 467

### N-{[(2R)-4-Benzylpiperazin-2yl]methyl}benzamide

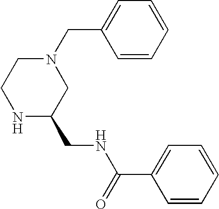

¹H-NMR (CDCl₃) δ 2.18 (1H, t), 2.30 (1H, t), 2.74 (1H, d), 2.88 (1H, d), 2.95 (1H, t), 3.14 (1H, d), 3.32-3.34 (1H, m), 3.47 (1H, d), 3.54 (1H, d), 3.60 (1H, d), 3.61 (1H, d), 5.47 (1H, br s), 7.26-7.49 (8H, m), 7.58 (1H, t), 7.80-7.82 (2H, m)

MS (ESI+, m/e) 310 (M+1)

### Reference Example 468

### N-{[(2R)-4-Benzylpiperazin-2-yl]methyl}cyclohexanecarboxamide

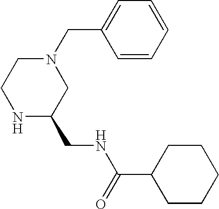

tert-Butyl (2S)-4-benzyl-2-{[(cyclohexylcarbonyl)(2,4-dimethoxybenzyl)amino]methyl}piperazine-1-carboxylate (2.26 g) was dissolved in dichloromethane (3.5 ml), TFA (15 ml) was added and the mixture was stirred at room temperature for 1.5 hr and at 70° C. for 10 min. The reaction mixture was poured into saturated aqueous sodium bicarbonate solution by small portions, and the mixture was basified by adding potassium carbonate by small portions, and the mixture was extracted with ethyl acetate (during which the insoluble material was filtered off). The extract was washed with brine, and dried over anhydrous magnesium sulfate. The solvent was evaporated in vacuo, and the mixture was concentrated to about 50 ml, the insoluble material was filtered off again. The filtrate was concentrated in vacuo, and the crystals were collected by filtration to give the desired product (473 mg).

¹H-NMR (CDCl₃) δ 1.17-1.85 (12H, m), 2.01-2.09 (2H, m), 2.68-2.74 (2H, m), 2.82-3.01 (3H, m), 3.16 (1H, ddd), 3.28 (1H, dt), 3.48 (2H, s), 5.88 (1H, br s), 7.23-7.34 (5H, m)

MS (ESI+, m/e) 316 (M+1)

### Reference Example 469

### (3R)-1-Benzyl-3-[(E)-2-pyridin-2-ylvinyl]piperazine dihydrochloride

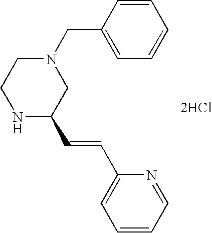

A 4 N hydrogen chloride-ethyl acetate solution (10 ml) was added to tert-butyl (2R)-4-benzyl-2-[(E)-2-pyridin-2-ylvinyl]piperazine-1-carboxylate (280 mg). After stirring at room temperature for 3 hr, the mixture was concentrated in vacuo, and the crystals were collected by filtration to give the desired product (260 mg).

MS (ESI+, m/e) 280 (M+1)

### Reference Example 470

### tert-Butyl 3-(2-hydroxy-2-methylpropyl)piperazine-1-carboxylate

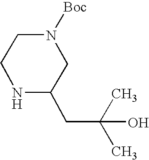

1-(1,4-Dibenzylpiperazin-2-yl)-2-methylpropan-2-ol (1.0 g) was dissolved in methanol (30 ml); 20% palladium hydroxide on carbon (containing 50% water) (200 mg) was added, and catalytic hydrogenation was performed at room temperature and atmospheric pressure for 17 hr. The catalyst was filtered off, and the filtrate was concentrated in vacuo. The residue and potassium carbonate (300 mg) were dissolved in THF (15 ml) and water (30 ml), and the solution was cooled to 0° C. (2Z)-{[(tert-Butoxycarbonyl)oxy]imino}(phenyl)acetonitrile (726 mg) was added thereto and the mixture was stirred at the same temperature for 1 hr and at room temperature for 3 hr. A 30% aqueous citric acid solution was added to the reaction mixture, and the mixture was washed twice with diethyl ether. The aqueous layer was saturated with potassium carbonate, and the mixture was extracted with ethyl acetate. The extract was washed with brine, dried over anhydrous magnesium sulfate, and concentrated in vacuo to give the desired product (500 mg) as an oil.

MS (ESI+, m/e) 259 (M+1)

### Reference Example 471

### tert-Butyl (3R)-3-(4-cyanobenzyl)piperazine-1-carboxylate

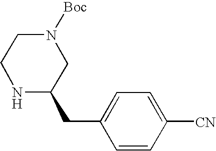

A solution of di-tert-butyl (2R)-2-(4-{[(trifluoromethyl)sulfonyl]oxy}benzyl)piperazine-1,4-dicarboxylate (1.05 g), zinc cyanide (282 mg), tetrakis(triphenylphosphine)palladium(0) (231 mg) and DMF (10 ml) was stirred at 80° C. for 15 hr. The insoluble material was filtered off, and the solvent was evaporated in vacuo. The residue was subjected to silica gel column chromatography, and the fraction eluted with ethyl acetate-hexane (1:9 to 3:7) was concentrated in vacuo to give di-tert-butyl (2R)-2-(4-cyanobenzyl)piperazine-1,4-dicarboxylate (570 mg) as crystals. The total amount thereof was dissolved in dichloromethane (1 ml), TFA (3 ml) was added. After stirring at room temperature for 1 hr, the mixture was concentrated in vacuo. The residue was neutralized by adding 6% aqueous sodium bicarbonate solution by small portions, and the mixture was extracted with chloroform. The extract was dried over anhydrous magnesium sulfate, and the solvent was evaporated in vacuo to give 4-[(2R)-piperazin-2-ylmethyl]benzonitrile (600 mg) as an oil. The total amount thereof and an aqueous sodium hydroxide solution (100 mg/10 ml) were dissolved in tert-butanol (10 ml) and the mixture was ice-cooled, and di-tert-butyl dicarbonate (546 mg) was added. After stirring at room temperature for 15 hr, the reaction mixture was concentrated in vacuo. The residue was subjected to silica gel column chromatography, and the fraction eluted with ethyl acetate-methanol (1:0 to 4:1) was concentrated in vacuo to give the desired product (145 mg) as an amorphous solid.

MS (ESI+, m/e) 302 (M+1)

### Reference Example 472

### (2R)-2-Benzyl-1,4-bis(trifluoroacetyl)piperazine

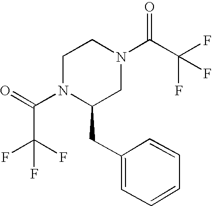

(2R)-2-Benzylpiperazine (14.9 g) was dissolved in toluene (150 ml), trifluoroacetic acid anhydride (35.7 g) was added, and the mixture was stirred at 70° C. for 1 hr. The reaction mixture was concentrated in vacuo, and the residue was dissolved in ethyl acetate (100 ml), and the solution was washed successively with 6% aqueous sodium bicarbonate solution and a 10% aqueous citric acid solution (each 50 ml). The solution was dried over anhydrous magnesium sulfate and concentrated in vacuo to give the desired product (28.7 g) as crystals.

MS (ESI+, m/e) 369 (M+1)

### Reference Example 473

### tert-Butyl (3R)-3-[4-(aminosulfonyl)benzyl]piperazine-1-carboxylate

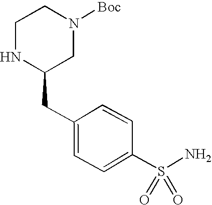

(2R)-2-Benzyl-1,4-bis(trifluoroacetyl)piperazine (2.2 g) was added to chlorosulfonic acid (4.8 g) by small portions over 5 min, and the mixture was stirred at room temperature for 2 hr. The reaction mixture was poured into a mixture of ethyl acetate-water (2:1, 30 ml) cooled to 5-10° C. The organic layer was dried over anhydrous magnesium sulfate and concentrated in vacuo. The residue was dissolved in THF (10 ml) and, after cooling again to 5-10° C., 25% aqueous ammonia (1.63 g) was added. After stirring at the same temperature for 30 min, the mixture was concentrated in vacuo, and a solution of potassium carbonate (4.2 g) in water (20 ml) and methanol (20 ml) were added to the residue. The mixture was stirred at room temperature for additional 15 hr and concentrated in vacuo, and methanol (10 ml) was added to the residue. The insoluble material was filtered off, and the filtrate was concentrated in vacuo. The residue was subjected to silica gel column chromatography, and the fraction eluted with ethyl acetate-methanol (9:1 to 7:3) was concentrated in vacuo to give 4-[(2R)-piperazin-2-ylmethyl]benzenesulfonamide (1.2 g) as crystals. A 1.02 g portion thereof and N,N-diisopropylethylamine (1.03 g) were dissolved in THF (20 ml) and the mixture was ice-cooled, di-tert-butyl dicarbonate (873 mg) was added, and the mixture was stirred at room temperature for 2 hr. The reaction mixture was concentrated in vacuo, the residue was subjected to silica gel column chromatography, and the fraction eluted with ethyl acetate-methanol (1:0 to 4:1) was concentrated in vacuo to give the desired product (1.3 g) as an amorphous solid.

MS (ESI+, m/e) 356 (M+1)

### Reference Example 474

### tert-Butyl 3-[2-(benzyloxy)ethyl]piperazine-1-carboxylate

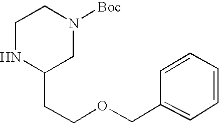

2-(1,4-Dibenzylpiperazin-2-yl)ethanol (931 mg) and benzyl bromide (513 mg) were dissolved in DMF (10 ml), and sodium hydride (60% in oil) (120 mg) was added at room temperature. After stirring at room temperature for 15 hr, the mixture was concentrated in vacuo. The residue was dissolved in ethyl acetate (20 ml), and the solution was washed successively with water and brine, dried over anhydrous magnesium sulfate, and concentrated in vacuo. The residue was subjected to silica gel column chromatography, and the fraction eluted with ethyl acetate-hexane (1:9 to 3:7) was concentrated in vacuo to give 1,4-dibenzyl-2-[2-(benzyloxy)ethyl]piperazine (830 mg) as an oil. A 801 mg portion thereof was dissolved in methanol (10 ml), 20% palladium hydroxide on carbon (containing 50% water) (400 mg) was added, and a catalytic hydrogenation was performed at room temperature and atmospheric pressure for 15 hr. The catalyst was filtered off, and the filtrate was concentrated in vacuo to give 2-[2-(benzyloxy)ethyl]piperazine (400 mg) as an oil. The total amount thereof and N,N-diisopropylethylamine (1.03 g) were dissolved in THF (20 ml) and the mixture was ice-cooled. Di-tert-butyl dicarbonate (873 mg) was added, and the mixture was stirred at room temperature for 2 hr. The reaction mixture was concentrated in vacuo, the residue was subjected to silica gel column chromatography, and the fraction eluted with ethyl acetate-methanol (1:0 to 4:1) was concentrated in vacuo to give the desired product (360 mg) as an oil.

MS (ESI+, m/e) 321 (M+1)

### Reference Example 475

### tert-Butyl (3R)-3-[4-(2,2,2-trifluoro-1-hydroxyethyl)benzyl]piperazine-1-carboxylate

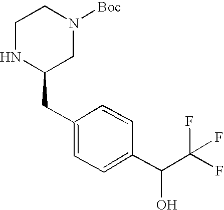

Di-tert-butyl (2R)-2-[4-(ethoxycarbonyl)benzyl]piperazine-1,4-dicarboxylate (1.79 g) was dissolved in ethanol (15 ml), pulverized potassium hydroxide (673 mg) was added and the mixture was stirred at 80° C. for 30 min. The reaction mixture was concentrated in vacuo, and the residue was dissolved in water (5 ml), and the mixture was weakly acidified (pH 3-4) with a 10% aqueous citric acid solution, and extracted with ethyl acetate. The extract was washed with brine, dried over anhydrous magnesium sulfate and concentrated in vacuo to give 4-{[(2R)-1,4-bis(tert-butoxycarbonyl)piperazin-2-yl]methyl}benzoic acid (1.67 g) as crystals. A 1.65 g portion thereof was dissolved in THF (15 ml) and the mixture was ice-cooled. N-Methylmorpholine (435 mg) and ethyl chloroformate (467 mg) were successively added. After stirring at 0-5° C. for 1 hr, the mixture was concentrated in vacuo, and the residue was dissolved in ethyl acetate (30 ml). The mixture was washed successively with 6% aqueous sodium bicarbonate solution and water, dried over anhydrous magnesium sulfate and concentrated in vacuo. The residue was subjected to silica gel column chromatography, and the fraction eluted with ethyl acetate-hexane (1:9 to 3:7) was concentrated in vacuo to give di-tert-butyl (2R)-2-(4-{[(ethoxycarbonyl)oxy]carbonyl}benzyl)piperazine-1,4-dicarboxylate (1.48 g) as an oil. The total amount thereof was dissolved in THF (15 ml) and the mixture was ice-cooled. Sodium borohydride (379 mg) was added, and methanol (3 ml) was added dropwise over 5 min. After stirring at the same temperature 30 min, a saturated aqueous ammonium chloride solution (5 ml) was added. The mixture was extracted with ethyl acetate, and the extract was dried over anhydrous magnesium sulfate and concentrated in vacuo to give di-tert-butyl (2R)-2-[4-(hydroxymethyl)benzyl]piperazine-1,4-dicarboxylate (1.11 g) as an amorphous solid. A 1.10 g portion thereof was dissolved in dichloromethane (20 ml) and manganese dioxide (2.35 g) was added. After stirring at room temperature for 15 hr, the insoluble material was filtered off, and the filtrate was concentrated in vacuo to give di-tert-butyl (2R)-2-(4-formylbenzyl)piperazine-1,4-dicarboxylate (1.01 g) as an oil. A 1.00 g portion thereof and trimethyl(trifluoromethyl)silane (702 mg) were dissolved in THF (10 ml), and TBAF (severl mg) was added. After stirring at room temperature for 2 hr, the mixture was concentrated in vacuo to give di-tert-butyl (2R)-2-[4-(2,2,2-trifluoro-1-hydroxyethyl)benzyl]piperazine-1,4-dicarboxylate (1.35 g) as an oil. TFA (3 ml) was added to the total amount thereof, and the mixture was stirred at room temperature for 30 min and concentrated in vacuo. The residue was dissolved in THF (15 ml) and the mixture was ice-cooled. N,N-Diisopropylethylamine (1.28 g) and di-tert-butyl dicarbonate (539 mg) were successively added, and the mixture was stirred at room temperature for 15 hr. The reaction mixture was concentrated in vacuo. The residue was subjected to silica gel column chromatography, and the fraction eluted with ethyl acetate-methanol (9:1 to 7:3) was concentrated in vacuo to give the desired product (0.9 g) as an amorphous solid.

MS (ESI+, m/e) 375 (M+1)

### Reference Example 476

### tert-Butyl (3S)-4-benzyl-3-(hydroxymethyl)piperazine-1-carboxylate

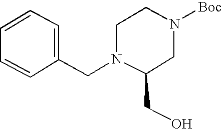

tert-Butyl (3S)-3-(hydroxymethyl)piperazine-1-carboxylate (15.1 g), benzaldehyde (7.4 g) and acetic acid (4.2 g) were dissolved in 1,2-dichloroethane (200 ml) and the mixture was ice-cooled. Sodium triacetoxyborohydride (19.3 g) was added and the mixture was stirred at room temperature for 15 hr. The mixture was neutralized with saturated aqueous sodium bicarbonate solution, and the organic layer was dried over anhydrous magnesium sulfate and concentrated in vacuo. The residue was subjected to silica gel column chromatography, and the fraction eluted with ethyl acetate-hexane (1:4 to 1:1) was concentrated in vacuo to give the desired product (16.1 g) as crystals.

MS (ESI+, m/e) 307 (M+1)

### Reference Example 477

### tert-Butyl (3S)-3-({[4-(methylsulfonyl)benzyl]oxy}methyl)piperazine-1-carboxylate

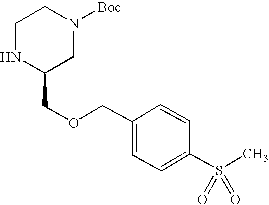

A solution of tert-butyl (3S)-4-benzyl-3-(hydroxymethyl)piperazine-1-carboxylate (1.53 g) in DMF (10 ml) was ice-cooled, and 1-(bromomethyl)-4-(methylthio)benzene (1.19 g) and sodium hydride (60% in oil) (220 mg) were added. After stirring at room temperature for 15 hr, the mixture was concentrated in vacuo and the residue was dissolved in ethyl acetate (30 ml). The solution was washed with brine, dried over anhydrous magnesium sulfate and concentrated in vacuo. The residue was subjected to silica gel column chromatography, and the fraction eluted with ethyl acetate-hexane (1:9 to 3:7) was concentrated in vacuo to give tert-butyl (3S)-4-benzyl-3-({[4-(methylthio)benzyl]oxy}methyl)piperazine-1-carboxylate (2.15 g) as an oil. A 1.05 g portion thereof was dissolved in methanol (3 ml) and the mixture was ice-cooled. 1 N Hydrochloric acid (3 ml) was added, and then a solution of m-chloroperbenzoic acid (1.17 g) in THF (1 ml) was added. After stirring at room temperature for 30 min, the mixture was neutralized with 6% aqueous sodium bicarbonate solution, and extracted with ethyl acetate. The extract was dried over anhydrous magnesium sulfate and concentrated in vacuo. The residue was subjected to silica gel column chromatography, and the fraction eluted with ethyl acetate-hexane (1:9 to 1:1) was concentrated in vacuo to give tert-butyl (3S)-4-benzyl-3-({[4-(methylsulfonyl)benzyl]oxy}methyl)piperazine-1-carboxylate (460 mg) as an oil. A 450 mg portion thereof was dissolved in 1,2-dichloroethane 15 ml), 1-chloroethyl chloroformate (163 mg) was added and, after heating under reflux for 5 hr, the mixture was concentrated in vacuo. Methanol (5 ml) was added to the residue, and the mixture was further heated under reflux for 4 hr. The reaction mixture was concentrated in vacuo, and the residue was neutralized with 6% aqueous sodium bicarbonate solution, and the mixture was extracted with ethyl acetate. The extract dried over anhydrous magnesium sulfate, and concentrated in vacuo. The residue was subjected to basic silica gel column chromatography, and the fraction eluted with ethyl acetate-hexane (1:1 to 4:1) was concentrated in vacuo to give the desired product (185 mg) as an oil.

MS (ESI+, m/e) 385 (M+1)

### Reference Example 478

### tert-Butyl (3S)-3-[(isopropylthio)methyl]piperazine-1-carboxylate

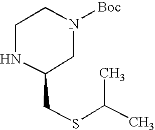

Triphenylphosphine (9.4 g) and carbon tetrabromide (11.9 g) were suspended in diethyl ether (200 ml), tert-butyl (3S)-4-benzyl-3-(hydroxymethyl)piperazine-1-carboxylate (9.2 g) was added by small portions over 5 min, and the mixture was stirred at room temperature for 15 hr. The insoluble material was filtered off, and the filtrate was concentrated in vacuo. The residue was subjected to silica gel column chromatography, and the fraction eluted with ethyl acetate-hexane (1:9 to 3:7) was concentrated in vacuo to give tert-butyl (3S)-4-benzyl-3-(bromomethyl)piperazine-1-carboxylate (8.5 g) as an oil. A 3.69 g portion thereof was dissolved in DMF (30 ml), sodium propane-2-thiolate (1.47 g) was added, and the mixture was stirred at room temperature for 15 hr. 6% Aqueous sodium bicarbonate solution (45 ml) was added to the reaction mixture, and the mixture was extracted with ethyl acetate. The extract was dried over anhydrous magnesium sulfate and concentrated in vacuo. The residue was subjected to silica gel column chromatography, and the fraction eluted with ethyl acetate-hexane (1:9 to 3:7) was concentrated in vacuo to give tert-butyl (3S)-4-benzyl-3-[(isopropylthio)methyl]piperazine-1-carboxylate (2.7 g) as an oil. A 900 mg portion thereof was dissolved in 1,2-dichloroethane (10 ml), 1-chloroethyl chloroformate (429 mg) was added and, after heating under reflux for 5 hr, the mixture was concentrated in vacuo. Methanol (10 ml) was added to the residue, and the mixture was further heated under reflux for 4 hr. The reaction mixture was concentrated in vacuo, and the residue was neutralized with 6% aqueous sodium bicarbonate solution, and the mixture was extracted with ethyl acetate. The extract was dried over anhydrous magnesium sulfate, and concentrated in vacuo. The residue was subjected to silica gel column chromatography, and the fraction eluted with ethyl acetate-methanol (1:0 to 9:1) was concentrated in vacuo to give the desired product (210 mg) as an oil.

MS (ESI+, m/e) 275 (M+1)

In the same manner as in Reference Example 478, the following compounds (Reference Examples 479 and 480) were obtained.

### Reference Example 479

### tert-Butyl 3-[(phenylthio)methyl]piperazine-1-carboxylate

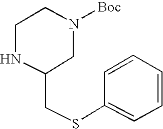

MS (ESI+, m/e) 309 (M+1)

### Reference Example 480

### tert-Butyl (3S)-3-[(phenylthio)methyl]piperazine-1-carboxylate

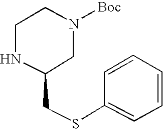

MS (ESI+, m/e) 309 (M+1)

### Reference Example 481

### tert-Butyl (3S)-3-{[4-(trifluoromethyl)phenoxy]methyl}piperazine-1-carboxylate

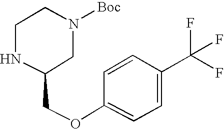

tert-Butyl (3S)-4-benzyl-3-(hydroxymethyl)piperazine-1-carboxylate (613 mg), 4-(trifluoromethyl)phenol (486 mg) and triphenylphosphine (787 mg) were dissolved in toluene (10 ml), DEAD (40% toluene solution, 1.3 g) was added and the mixture was stirred at room temperature for 15 hr. The insoluble material was filtered off and the filtrate was concentrated in vacuo. The residue was subjected to silica gel column chromatography, and the fraction eluted with ethyl acetate-hexane (1:9 to 3:7) was concentrated in vacuo to give tert-butyl (3S)-4-benzyl-3-{[4-(trifluoromethyl)phenoxy]methyl}piperazine-1-carboxylate (310 mg) as an amorphous solid. A 305 mg portion thereof was dissolved in methanol-THF (2:1, 4.5 ml), 20% palladium hydroxide on carbon (containing 50% water) (110 mg) was added, and a catalytic hydrogenation was performed at room temperature and atmospheric pressure for 15 hr. The catalyst was filtered off, and the filtrate was concentrated in vacuo to give the desired product (240 mg) as an oil.

MS (ESI+, m/e) 361 (M+1)

### Reference Example 482

### tert-Butyl (3S)-3-{[4-(1-hydroxyethyl)phenoxy]methyl}piperazine-1-carboxylate

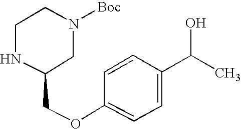

tert-Butyl (3S)-4-benzyl-3-(hydroxymethyl)piperazine-1-carboxylate (920 mg), 4-hydroxyacetophenone (613 mg) and triphenylphosphine (1.18 g) were dissolved in toluene (15 ml), DEAD (40% toluene solution, 1.96 g) was added and the mixture was stirred at room temperature for 2 hr. The insoluble material was filtered off and the filtrate was concentrated in vacuo. The residue was subjected to silica gel column chromatography, and the fraction eluted with ethyl acetate-hexane (1:9 to 3:7) was concentrated in vacuo to give tert-butyl (3S)-3-[(4-acetylphenoxy)methyl]-4-benzylpiperazine-1-carboxylate (535 mg) as an amorphous solid. The total amount thereof was dissolved in methanol (10 ml), 20% palladium hydroxide on carbon (containing 50% water) (900 mg) was added, and a catalytic hydrogenation was performed at room temperature and atmospheric pressure for 15 hr. The catalyst was filtered off, and the filtrate was concentrated in vacuo to give the desired product (405 mg) as an amorphous solid.

MS (ESI+, m/e) 337 (M+1)

### Reference Example 483

### tert-Butyl (3S)-3-[(4-cyanophenoxy)methyl]piperazine-1-carboxylate

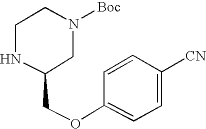

tert-Butyl (3S)-4-benzyl-3-(hydroxymethyl)piperazine-1-carboxylate (613 mg), 4-hydroxybenzonitrile (357 mg) and triphenylphosphine (787 mg) were dissolved in toluene (10 ml), DEAD (40% toluene solution, 1.3 g) was added and the mixture was stirred at room temperature for 2 hr. The insoluble material was filtered off, and the filtrate was concentrated in vacuo. The residue was subjected to silica gel column chromatography, and the fraction eluted with ethyl acetate-hexane (1:9 to 3:7) was concentrated in vacuo to give tert-butyl (3S)-4-benzyl-3-[(4-cyanophenoxy)methyl]piperazine-1-carboxylate (485 mg) as an amorphous solid. The total amount thereof was dissolved in 1,2-dichloroethane (5 ml), 1-chloroethyl chloroformate (187 mg) was added and, after heating under reflux for 5 hr, the mixture was concentrated in vacuo. Methanol (5 ml) was added to the residue and, after heating under reflux for additional 3 hr, the mixture was concentrated in vacuo. The residue was neutralized with 6% aqueous sodium bicarbonate solution, and the mixture was extracted with ethyl acetate. The extract was dried over anhydrous magnesium sulfate, and concentrated in vacuo. The residue was subjected to basic silica gel column chromatography, and the fraction eluted with ethyl acetate-hexane (1:4 to 1:1) was concentrated in vacuo to give the desired product (130 mg) as an oil.

MS (ESI+, m/e) 318 (M+1)

### Reference Example 484

### tert-Butyl [2-(4-{[(2R)-2,4-dibenzylpiperazin-1-yl]carbonyl}-5-phenyl-1H-imidazol-1-yl)ethyl)(tetrahydro-2H-pyran-4-yl)carbamate

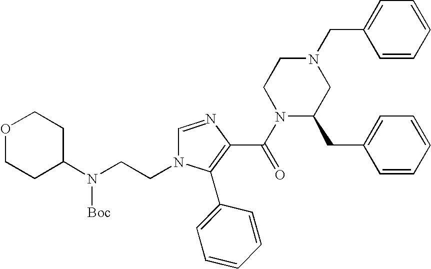

A solution of 1-{2-[(tert-butoxycarbonyl)(tetrahydro-2H-pyran-4-yl)amino]ethyl}-5-phenyl-1H-imidazole-4-carboxylic acid (3.68 g), (3R)-1,3-dibenzylpiperazine (2.36 g), WSC.HCl (2.04 g), HOBt (1.32 g) and DMF (45 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane (1:2 to 1:0) was concentrated in vacuo to give the desired product (5.44 g) as an amorphous solid.

MS (ESI+, m/e) 664 (M+1)

In the same manner as in Reference Example 484, the following compounds (Reference Examples 485 to 492) were obtained.

### Reference Example 485

### 4-[((2R)-4-Benzyl-1-([1-(3-morpholinophenyl)-5-phenyl-1H-imidazol-4-yl]carbonyl}piperazin-2-yl)methyl]benzonitrile

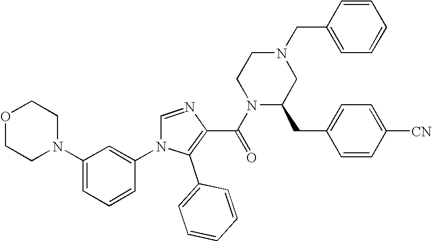

MS (ESI+, m/e) 623 (M+1)

### Reference Example 486

### tert-Butyl (3R)-3-(4-cyanobenzyl)-4-{[1-(3-morpholinophenyl)-5-phenyl-1H-imidazol-4-yl]carbonyl}piperazine-1-carboxylate

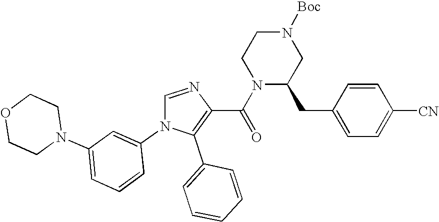

MS (ESI+, m/e) 633 (M+1)

### Reference Example 487

### Ethyl 4-[((2R)-4-benzyl-1-{[1-(2,3-dihydro-1H-inden-2-yl)-5-phenyl-1H-imidazol-4-yl]carbonyl}piperazin-2-yl)methyl]benzoate

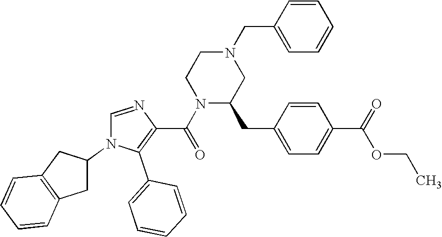

MS (ESI+, m/e) 625 (M+1)

### Reference Example 488

### tert-Butyl (3S)-4-[(2-ethoxy-1,5-diphenyl-1H-imidazol-4-yl)carbonyl]-3-[(phenylthio)methyl]piperazine-1-carboxylate

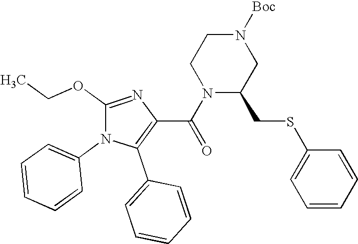

MS (ESI+, m/e) 599 (M+1)

### Reference Example 489

### 4-({(2R)-4-Benzyl-1-[(5-methyl-1,2-diphenyl-1H-pyrrol-3-yl)carbonyl]piperazin-2-yl}methyl)phenol

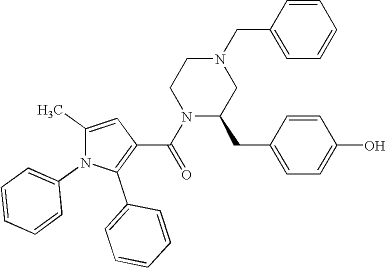

MS (ESI+, m/e) 542 (M+1)

### Reference Example 490

### tert-Butyl (3R)-3-benzyl-4-{[1-(1-benzylpyrrolidin-3-yl)-5-phenyl-1H-imidazol-4-yl]carbonyl}piperazine-1-carboxylate

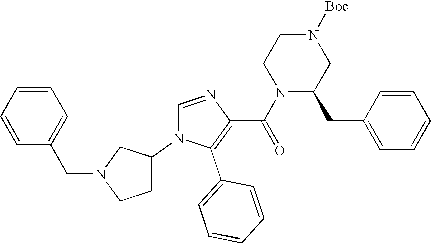

MS (ESI+, m/e) 606 (M+1)

### Reference Example 491

### tert-Butyl (3R)-3-benzyl-4-[(3,4-diphenyl-1H-pyrazol-5-yl)carbonyl]piperazine-1-carboxylate

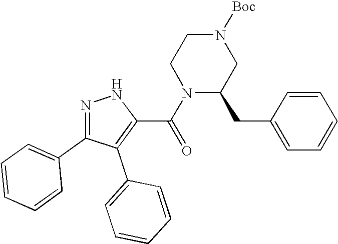

MS (ESI+, m/e) 523 (M+1)

### Reference Example 492

### tert-Butyl (3R)-3-benzyl-4-{[4-(3-bromophenyl)-5-formyl-3-phenyl-1H-pyrrol-2-yl]carbonyl}piperazine-1-carboxylate

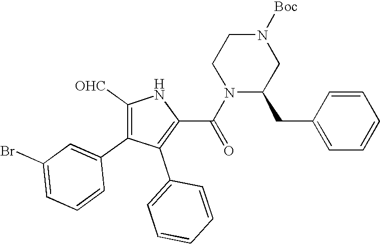

MS (ESI+, m/e) 628 (M+1)

### Reference Example 493

### (1S)-2-((2R)-4-Benzyl-1-{[2-ethoxy-1-(2-methoxyphenyl)-5-phenyl-1H-imidazol-4-yl]carbonyl}piperazin-2-yl)-1-cyclopropylethanol and (1R)-2-((2R)-4-benzyl-1-{[2-ethoxy-1-(2-methoxyphenyl)-5-phenyl-1H-imidazol-4-yl]carbonyl}piperazin-2-yl)-1-cyclopropylethanol

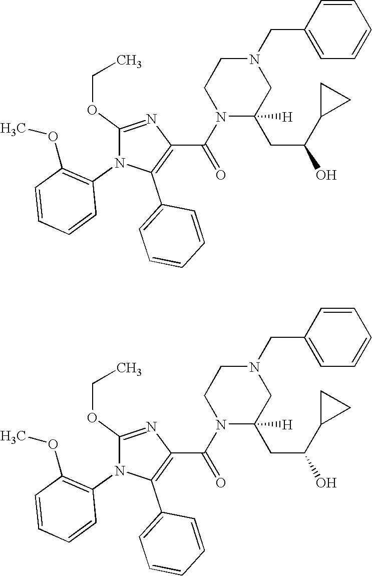

A solution of 2-ethoxy-1-(2-methoxyphenyl)-5-phenyl-1H-imidazole-4-carboxylic acid (190 mg), 1-benzyl-3-(2-cyclopropylethyl)piperazine (295 mg), WSC.HCl (215 mg), HOBt (40 mg), triethylamine (200 μl) and dichloromethane (5 ml) was stirred at room temperature for 1 day, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography and, of the fractions eluted with ethyl acetate-hexane (1:4 to 1:0), a less polar fraction was concentrated in vacuo to give. (1S)-2-((2R)-4-benzyl-1-{[2-ethoxy-1-(2-methoxyphenyl)-5-phenyl-1H-imidazol-4-yl]carbonyl}piperazin-2-yl)-1-cyclopropylethanol (9.6 mg).

MS (ESI+, m/e) 581 (M+1)

The more polar fraction obtained by the above-mentioned column chromatography was concentrated in vacuo to give (1R)-2-((2R)-4-benzyl-1-{[2-ethoxy-1-(2-methoxyphenyl)-5-phenyl-1H-imidazol-4-yl]carbonyl}piperazin-2-yl)-1-cyclopropylethanol (72 mg).

MS (ESI+, m/e) 581 (M+1)

### Reference Example 494

### tert-Butyl (3R)-3-benzyl-4-{[5-phenyl-1-(trans-4-hydroxytetrahydro-2H-pyran-3-yl)-1H-imidazol-4-yl]carbonyl}piperazine-1-carboxylate and tert-butyl (3R)-3-benzyl-4-{[5-phenyl-1-(cis-4-hydroxytetrahydro-2H-pyran-3-yl)-1H-imidazol-4-yl]carbonyl}piperazine-1-carboxylate

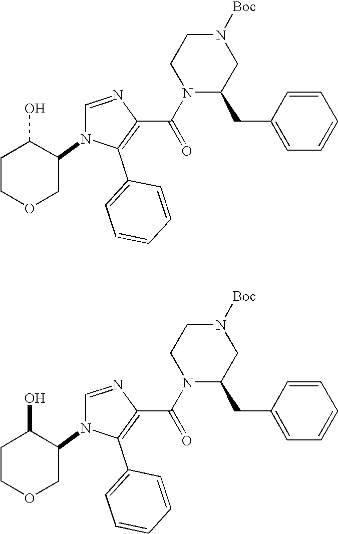

A solution of 5-phenyl-1-(4-hydroxytetrahydro-2H-pyran-3-yl)-1H-imidazole-4-carboxylic acid (1.1 g), tert-butyl (3R)-3-benzylpiperazine-1-carboxylate (1.3 g), WSC.HCl (1.5 g), HOBt (2.3 g) and DMF (30 ml) was stirred at room temperature for 12 hr, and poured into saturated aqueous sodium bicarbonate solution, and the mixture was extracted with ethyl acetate. The extract was washed with brine, and dried over anhydrous sodium sulfate, and the solvent was evaporated in vacuo. The residue was subjected to silica gel column chromatography, and a less polar fraction eluted with ethyl acetate was concentrated in vacuo to give the desired product (650 mg) and a more polar fraction was concentrated in vacuo to give the desired product (260 mg), each as an amorphous solid.

MS (ESI+, m/e) 547 (M+1)

MS (ESI+, m/e) 547 (M+1)

### Reference Example 495

### tert-Butyl (3R)-3-benzyl-4-[(1-{1-[(benzyloxy)carbonyl]piperidin-3-yl}-5-phenyl-1H-imidazol-4-yl)carbonyl]piperazine-1-carboxylate

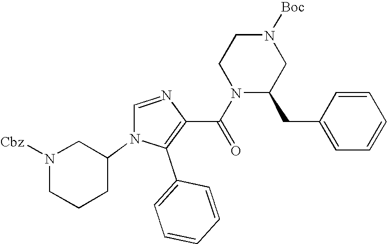

1-{1-[(Benzyloxy)carbonyl]piperidin-3-yl}-5-phenyl-1H-imidazole-4-carboxylic acid (3.00 g) was dissolved in DMF (50 ml), tert-butyl (3R)-3-benzylpiperazine-1-carboxylate (2.45 g), WSC.HCl (2.13 g) and HOBt (1.36 g) were added and the mixture was stirred at 60° C. for 3 hr. The reaction mixture was poured into saturated aqueous sodium bicarbonate solution, and the mixture was extracted with ethyl acetate. The extract was washed successively with water and brine, and dried over anhydrous sodium sulfate, and the solvent was evaporated in vacuo. The residue was subjected to silica gel column chromatography, and the fraction eluted with ethyl acetate was concentrated in vacuo to give the desired product (4.51 g) as an amorphous solid.

MS (ESI+, m/e) 664 (M+1)

In the same manner as in Reference Example 495, the following compound (Reference Example 496) was obtained.

### Reference Example 496

### tert-Butyl (3R)-3-benzyl-4-{[1-(2-oxoazepan-3-yl)-5-phenyl-1H-imidazol-4-yl]carbonyl}piperazine-1-carboxylate

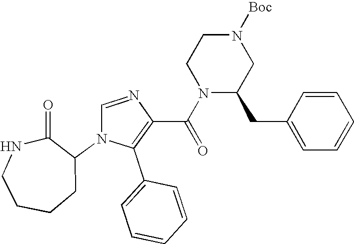

MS (ESI+, m/e) 558 (M+1)

### Reference Example 497

### 1-((2S)-4-Benzyl-1-{[1-(2,3-dihydro-1H-inden-2-yl)-5-phenyl-1H-imidazol-4-yl]carbonyl}piperazin-2-yl)-2-methylpropan-1-one

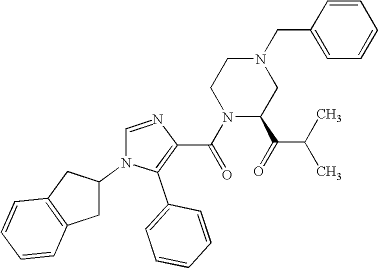

A solution of 1-(2,3-dihydro-1H-inden-2-yl)-5-phenyl-1H-imidazole-4-carboxylic acid (380 mg), 1-[(2S)-4-benzylpiperazin-2-yl]-2-methylpropan-1-one (308 mg), WSC.HCl (312 mg), HOBt (58 mg), N,N-diisopropylethylamine (0.44 ml), DMAP (39 mg) and DMF (4 ml) was stirred at room temperature for 12 hr, and poured into saturated aqueous sodium bicarbonate solution, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (2:1) was concentrated in vacuo to give the desired product (560 mg) as an amorphous solid.

MS (ESI+, m/e) 583 (M+1)

In the same manner as in Reference Example 497, the following compounds (Reference Examples 498 to 504) were obtained.

### Reference Example 498

### tert-Butyl (3R)-4-{[1-(2,3-dihydro-1H-inden-2-yl)-5-phenyl-1H-imidazol-4-yl]carbonyl}-3-isobutylpiperazine-1-carboxylate

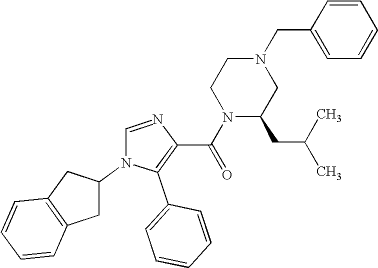

MS (ESI+, m/e) 519 (M+1)

### Reference Example 499

### (2S)-4-Benzyl-2-[(benzyloxy)methyl]-1-{[1-(2,3-dihydro-1H-inden-2-yl)-5-phenyl-1H-imidazol-4-yl]carbonyl}piperazine

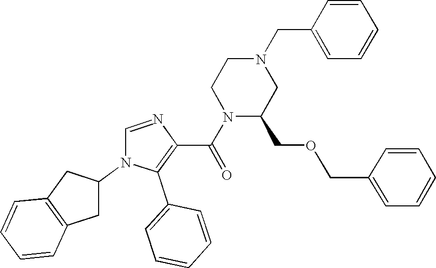

MS (ESI+, m/e) 583 (M+1)

### Reference Example 500

### Ethyl 4-[((2R)-4-Benzyl-1-{[1-(2,3-dihydro-1H-inden-2-yl)-5-phenyl-1H-imidazol-4-yl]carbonyl}piperazin-2-yl)methyl]benzoate

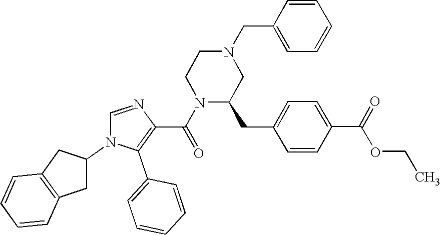

MS (ESI+, m/e) 625 (M+1)

### Reference Example 501

### 4-{3-[4-({(2S)-4-Benzyl-2-[(benzyloxy)methyl]piperazin-1-yl}carbonyl)-5-phenyl-1H-imidazol-1-yl]phenyl}morpholine

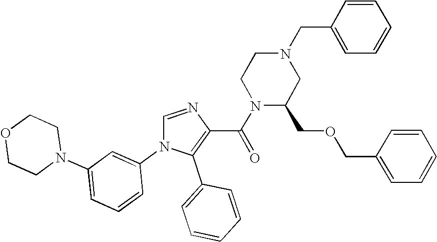

MS (ESI+, m/e) 628 (M+1)

### Reference Example 502

### 4-(3-(4-[((2S)-4-Benzyl-2-{[(2-methylprop-2-en-1-yl)oxy]methyl}piperazin-1-yl)carbonyl]-5-phenyl-1H-imidazol-1-yl}phenyl)morpholine

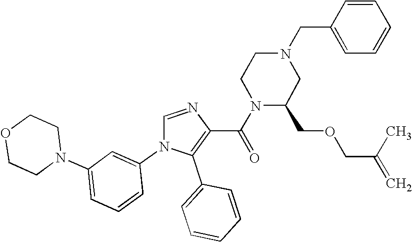

MS (ESI+, m/e) 592 (M+1)

### Reference Example 503

### (2S)-4-Benzyl-2-[(benzyloxy)methyl]-1-[(3,4-diphenyl-1H-pyrazol-5-yl)carbonyl]piperazine

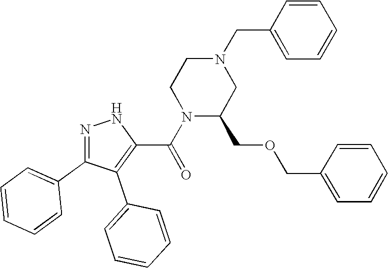

MS (ESI+, m/e) 543 (M+1)

### Reference Example 504

### (2R)-4-Benzyl-1-[(3,4-diphenyl-1H-pyrazol-5-yl)carbonyl]-2-isobutylpiperazine

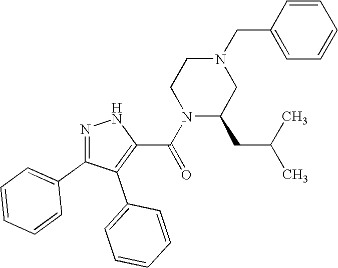

MS (ESI+, m/e) 479 (M+1)

### Reference Example 505

### (1R)-2-((2R)-4-Benzyl-1-{[1-(3-morpholinophenyl)-5-phenyl-1H-imidazol-4-yl)carbonyl}piperazin-2-yl)-1-cyclopropylethanol and (1S)-2-((2R)-4-benzyl-1-{[1-(3-morpholinophenyl)-5-phenyl-1H-imidazol-4-yl]carbonyl}piperazin-2-yl)-1-cyclopropylethanol

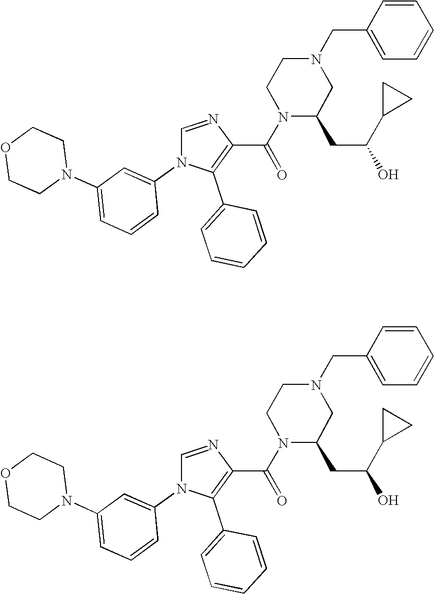

A solution of 1-(3-morpholinophenyl)-5-phenyl-1H-imidazole-4-carboxylic acid (201 mg), 2-[(2R)-4-benzylpiperazin-2-yl]-1-cyclopropylethanol (150 mg), WSC.HCl (312 mg), HOBt (58 mg), N,N-diisopropylethylamine (0.44 ml), DMAP (39 mg) and DMF (4 ml) was stirred at room temperature for 12 hr, and poured into saturated aqueous sodium bicarbonate solution, and the mixture was extracted with ethyl acetate. The extract was washed with brine, and dried over anhydrous sodium sulfate, and the solvent was evaporated in vacuo. The residue was subjected to silica gel column chromatography, a diastereomer was separated from the fraction eluted with ethyl acetate-methanol (4:1), and the fractions were each concentrated in vacuo to give the desired product (134 mg and 122 mg), each as an amorphous solid.

MS (ESI+, m/e) 592 (M+1)

MS (ESI+, m/e) 592 (M+1)

### Reference Example 506

### Lithium 2-methyl-1-(3-morpholinophenyl)-5-phenyl-1H-imidazole-4-carboxylate

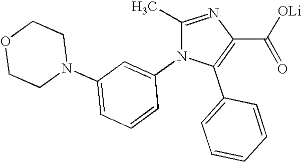

A mixture of ethyl 2-methyl-1-(3-morpholinophenyl)-5-phenyl-1H-imidazole-4-carboxylate (1.07 g), lithium hydroxide monohydrate (115 mg), THF (10 ml), ethanol (10 ml) and water (6 ml) was stirred at 70° C. for 8 hr. After cooling to room temperature, the mixture was concentrated in vacuo to give the desired product (1.06 g).

¹H-NMR (DMSO-d₆) δ 2.16 (3H, s), 2.99-3.02 (4H, m), 3.65-3.68 (4H, m), 6.56 (1H, dd), 6.72 (1H, dd), 6.90 (1H, dd), 7.14-7.23 (6H, in)

MS (ESI+, m/e) 364 (M+1−“Li”)

### Reference Example 507

### tert-Butyl (3R)-3-benzyl-4-{[2-methyl-1-(3-morpholinophenyl)-5-phenyl-1H-imidazol-4-yl]carbonyl}piperazine-1-carboxylate

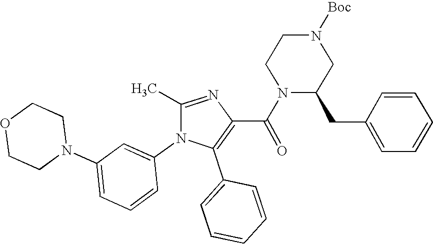

A solution of lithium 2-methyl-1-(3-morpholinophenyl)-5-phenyl-1H-imidazole-4-carboxylate (497 mg), tert-butyl (3R)-3-benzylpiperazine-1-carboxylate (450 mg), WSC.HCl (520 mg), HOBt (310 mg) and DMF (10 ml) was stirred at 55° C. for 3 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-methanol (1:0 to 19:1) was concentrated in vacuo to give the desired product (825 mg).

MS (ESI+, m/e) 622 (M+1)

### Reference Example 508

### tert-Butyl (3R)-3-benzyl-4-{[1-(1-methylpiperidin-4-yl)-5-phenyl-1H-imidazol-4-yl]carbonyl}piperazine-1-carboxylate

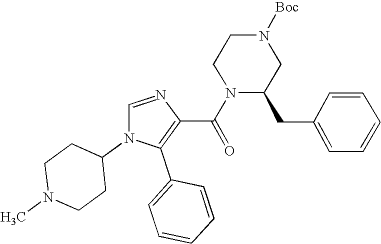

Methyl 1-(1-methylpiperidin-4-yl)-5-phenyl-1H-imidazole-4-carboxylate (186 mg) was dissolved in THF-ethanol (1:1, 4 ml), lithium hydroxide monohydrate (39 mg) and water (1 ml) were added and the mixture was stirred at 80° C. for 2 hr. The reaction mixture was concentrated in vacuo, the residue was suspended in ethanol and the mixture was concentrated again in vacuo. The residue was vacuum dried, suspended in DMF (8 ml), tert-butyl (3R)-3-benzylpiperazine-1-carboxylate (176 mg), WSC.HCl (131 mg) and HOBt (380 mg) were added, and the mixture was stirred at 60° C. for 3 hr. The reaction mixture was poured into saturated aqueous sodium bicarbonate solution, and the mixture was extracted with ethyl acetate. The extract was washed successively with water and brine, and dried over anhydrous sodium sulfate, and the solvent was evaporated in vacuo. The residue was subjected to silica gel column chromatography, and the fraction eluted with ethyl acetate-methanol (19:1) was concentrated in vacuo to give the desired product (155 mg) as an amorphous solid.

MS (ESI+, m/e) 544 (M+1)

In the same manner as in Reference Example 508, the following compounds (Reference Examples 509 to 533) were obtained.

### Reference Example 509

### tert-Butyl (3R)-3-benzyl-4-({1-[(3S)-1-benzylpyrrolidin-3-yl]-5-phenyl-1H-imidazol-4-yl}carbonyl)piperazine-1-carboxylate

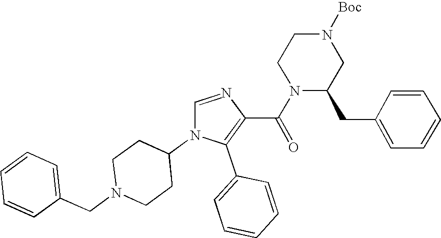

MS (ESI+, m/e) 620 (M+1)

### Reference Example 510

### tert-Butyl (3R)-3-benzyl-4-({1-[(3S)-1-benzylpyrrolidin-3-yl]-5-phenyl-1H-imidazol-4-yl}carbonyl)piperazine-1-carboxylate

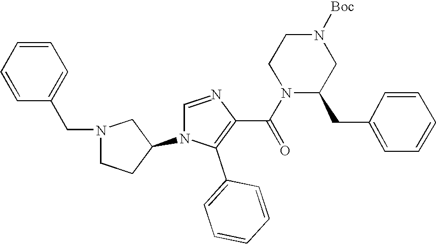

MS (ESI+, m/e) 606 (M+1)

### Reference Example 511

### tert-Butyl (3R)-3-benzyl-4-({1-[(3R)-1-benzylpyrrolidin-3-yl]-5-phenyl-1H-imidazol-4-yl}carbonyl)piperazine-1-carboxylate

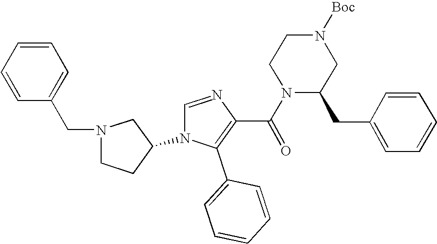

MS (ESI+, m/e) 606 (M+1)

### Reference Example 512

### ((2S,6R)-4,6-Dibenzyl-1-{[1-(3-morpholinophenyl)-5-phenyl-1H-imidazol-4-yl]carbonyl}piperazin-2-yl)methanol

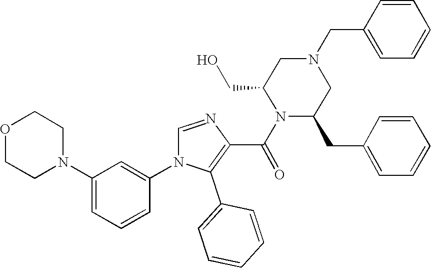

MS (ESI+, m/e) 628 (M+1)

### Reference Example 513

### tert-Butyl (3R)-3-benzyl-4-[(2-chloro-1,5-diphenyl-1H-imidazol-4-yl)carbonyl]piperazine-1-carboxylate

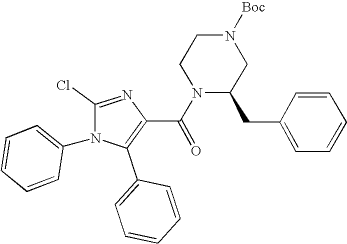

MS (ESI+, m/e) 557 (M+1)

### Reference Example 514

### tert-Butyl (3R)-3-benzyl-4-[(5-phenyl-1-{3-[benzyloxy]phenyl}-1H-imidazol-4-yl)carbonyl]piperazine-1-carboxylate

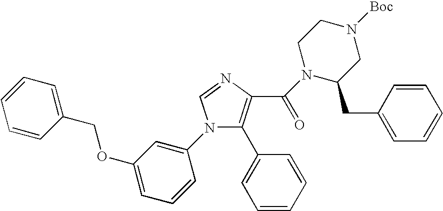

MS (ESI+, m/e) 629 (M+1)

### Reference Example 515

### tert-Butyl (3R)-3-benzyl-4-{[1-(2-oxo-1-phenylpiperidin-3-yl)-5-phenyl-1H-imidazol-4-yl]carbonyl}piperazine-1-carboxylate

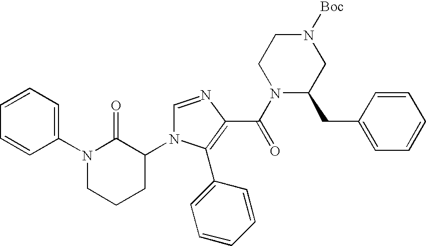

MS (ESI+, m/e) 620 (M+1)

### Reference Example 516

### tert-Butyl (3R)-3-benzyl-4-({1-[(1-methylpiperidin-2-yl)methyl]-5-phenyl-1H-imidazol-4-yl)carbonyl)piperazine-1-carboxylate

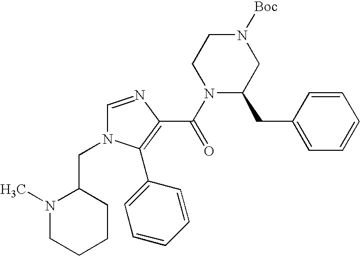

MS (ESI+, m/e) 558 (M+1)

### Reference Example 517

### tert-Butyl (3R)-3-benzyl-4-({1-((1S,2S)-2-hydroxy-1-(methoxymethyl)-2-phenylethyl]-5-phenyl-1H-imidazol-4-yl}carbonyl)piperazine-1-carboxylate

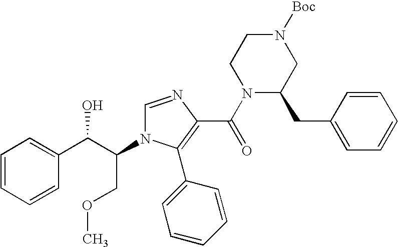

MS (ESI+, m/e) 611 (M+1)

### Reference Example 518

### tert-Butyl (3R)-3-benzyl-4-({1-[(1R)-1-benzyl-2-hydroxyethyl]-5-phenyl-1H-imidazol-4-yl}carbonyl)piperazine-1-carboxylate

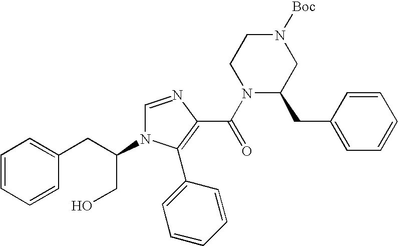

MS (ESI+, m/e) 581 (M+1)

### Reference Example 519

### tert-Butyl 2-[(4-{[(2R)-2,4-dibenzylpiperazin-1-yl]carbonyl}-5-phenyl-1H-imidazol-1-yl)methyl]piperidine-1-carboxylate

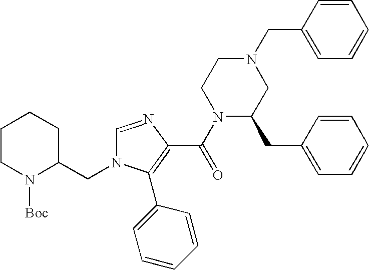

MS (ESI+, m/e) 634 (M+1)

### Reference Example 520

### tert-Butyl (3R)-3-benzyl-4-({1-[(1S,2S)-2-hydroxy-1-(hydroxymethyl)-2-phenylethyl]-5-phenyl-1H-imidazol-4-yl}carbonyl)piperazine-1-carboxylate

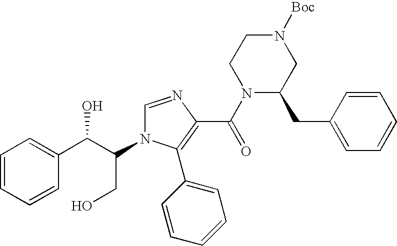

MS (ESI+, m/e) 597 (M+1)

### Reference Example 521

### tert-Butyl (3R)-3-benzyl-4-({1-[(1S,2R)-2-hydroxy-1,2-diphenylethyl]-5-phenyl-1H-imidazol-4-yl}carbonyl)piperazine-1-carboxylate

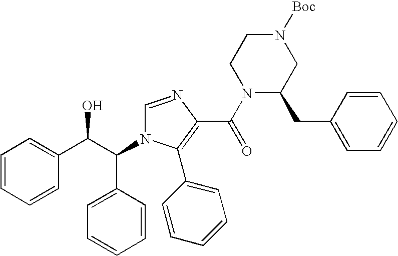

MS (ESI+, m/e) 643 (M+1)

### Reference Example 522

### tert-Butyl (3R)-3-benzyl-4-({1-[(1S)-2-hydroxy-2-methyl-1-phenylproyl]-5-phenyl-1H-imidazol-4-yl}carbonyl)piperazine-1-carboxylate

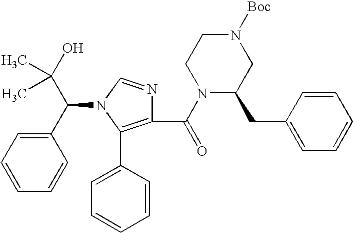

MS (ESI+, m/e) 595 (M+1)

### Reference Example 523

### tert-Butyl (3R)-3-benzyl-4-({1-[(1S)-2-ethyl-2-hydroxy-1-phenylbutyl]-5-phenyl-1H-imidazol-4-yl}carbonyl)piperazine-1-carboxylate

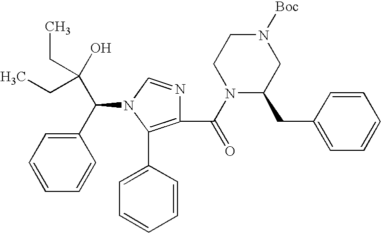

MS (ESI+, m/e) 623 (M+1)

### Reference Example 524

### tert-Butyl (3R)-3-benzyl-4-({1-[(1S)-2-butyl-2-hydroxy-1-phenylhexyl]-5-phenyl-1H-imidazol-4-yl}carbonyl)piperazine-1-carboxylate

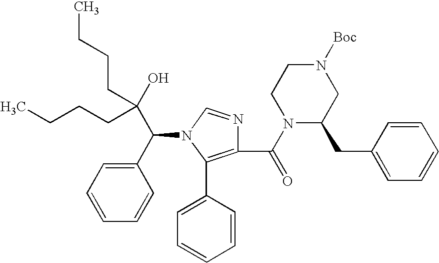

MS (ESI+, m/e) 679 (M+1)

### Reference Example 525

### tert-Butyl (3R)-3-benzyl-4-({1-[(1S)-2-hydroxy-1,2-dimethylpropyl]-5-phenyl-1H-imidazol-4-yl}carbonyl)piperazine-1-carboxylate

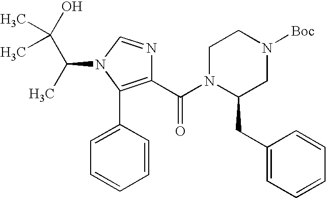

MS (ESI+, m/e) 533 (M+1)

### Reference Example 526

### tert-Butyl (3R)-3-benzyl-4-({1-[(1S)-2-hydroxy-1-methyl-2-phenylhexyl]-5-phenyl-1H-imidazol-4-yl}carbonyl)piperazine-1-carboxylate

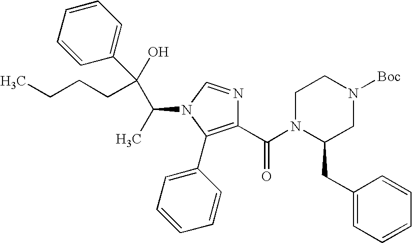

MS (ESI+, m/e) 637 (M+1)

### Reference Example 527

### tert-Butyl (3R)-3-benzyl-4-({1-[(1R,2S)-2-hydroxy-1,2-diphenylethyl]-5-phenyl-1H-imidazol-4-yl}carbonyl)piperazine-1-carboxylate

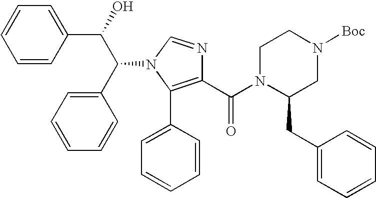

MS (ESI+, m/e) 643 (M+1)

### Reference Example 528

### tert-Butyl (3R)-3-benzyl-4-({2-ethoxy-1-[3-(methylsulfonyl)phenyl]-5-phenyl-1H-imidazol-4-yl}carbonyl)piperazine-1-carboxylate

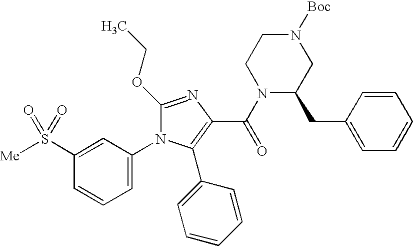

MS (ESI+, m/e) 645 (M+1)

### Reference Example 529

### tert-Butyl (3R)-3-benzyl-4-{[2-ethoxy-1-(3-morpholinophenyl)-5-phenyl-1H-imidazol-4-yl]carbonyl}piperazine-1-carboxylate

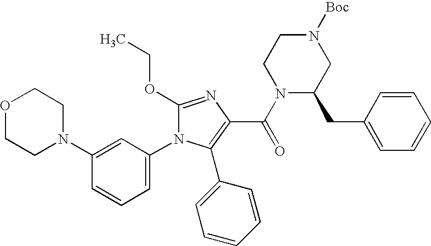

MS (ESI+, m/e) 652 (M+1)

### Reference Example 530

### tert-Butyl (3R)-3-benzyl-4-{[1-(2-hydroxy-2-methylpropyl)-5-phenyl-1H-imidazol-4-yl]carbonyl}piperazine-1-carboxylate

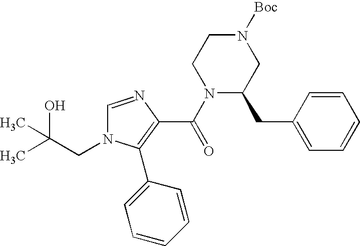

MS (ESI+, m/e) 604 (M+1)

### Reference Example 531

### tert-Butyl (3R)-3-benzyl-4-({1-[(1-benzyl-4-hydroxypiperidin-4-yl)methyl]-5-phenyl-1H-imidazol-4-yl}carbonyl)piperazine-1-carboxylate

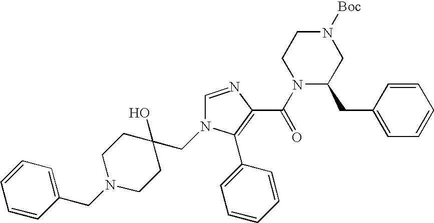

MS (ESI+, m/e) 650 (M+1)

### Reference Example 532

### tert-Butyl (3R)-3-benzyl-4-({1-[(1-benzyl-3-hydroxypiperidin-3-yl)methyl]-5-phenyl-1H-imidazol-4-yl}carbonyl)piperazine-1-carboxylate

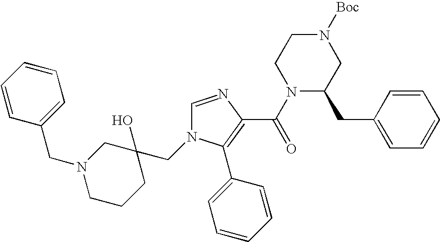

MS (ESI+, m/e) 650 (M+1)

### Reference Example 533

### tert-Butyl (3R)-3-benzyl-4-({1-[(4-hydroxytetrahydro-2H-pyran-4-yl)methyl]-5-phenyl-1H-imidazol-4-yl}carbonyl)piperazine-1-carboxylate

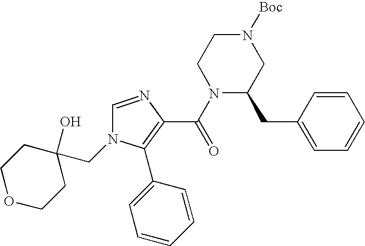

MS (ESI+, m/e) 561 (M+1)

### Reference Example 534

### tert-Butyl (3R)-3-benzyl-4-{[1-(2-oxopiperidin-1-yl)-5-phenyl-1H-imidazol-4-yl]carbonyl)piperazine-1-carboxylate

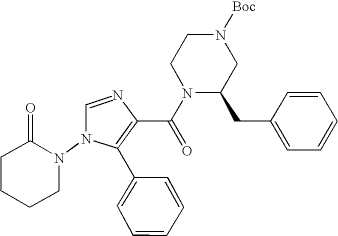

Methyl 1-(2-oxopiperidin-1-yl)-5-phenyl-1H-imidazole-4-carboxylate (312 mg) was dissolved in a mixed solvent of to ethanol (5 ml) and water (3 ml), lithium hydroxide monohydrate (65 mg) was added and the mixture was stirred at 70° C. for 1 hr. The reaction mixture was concentrated in vacuo, the residue was suspended in ethanol, and the mixture was concentrated again in vacuo, and the residue was vacuum dried. This was is suspended in DMF (10 ml), tert-butyl (3R)-3-benzylpiperazine-1-carboxylate (345 mg), WSC.HCl (399 mg) and HOBt (637 mg) were added, and the mixture was stirred at room temperature for 12 hr. The reaction mixture was poured into saturated aqueous sodium bicarbonate solution, and the mixture was extracted with ethyl acetate. The extract was washed successively with water and brine, and dried over anhydrous sodium sulfate, and the solvent was evaporated in vacuo. The residue was subjected to basic silica gel column chromatography, and the fraction eluted with ethyl acetate was concentrated in vacuo to give the desired product (511 mg) as an amorphous solid.

MS (ESI+, m/e) 544 (M+1)

### Reference Example 535

### tert-Butyl (3R)-4-({1-[(1-acetylpiperidin-2-yl)methyl]-5-phenyl-1H-imidazol-4-yl)carbonyl)-3-benzylpiperazine-1-carboxylate

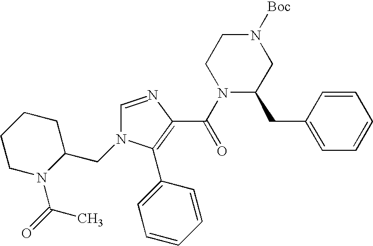

A mixture of ethyl 5-phenyl-1-(piperidin-2-ylmethyl)-1H-imidazole-4-carboxylate (containing a trace amount of ethyl acetate) (950 mg), lithium hydroxide monohydrate (260 mg), ethanol (6 ml) and water (6 ml) was stirred at 80° C. for 12 hr, and concentrated in vacuo. A solution of the total amount of the residue and tert-butyl (3R)-3-benzylpiperazine-1-carboxylate (1.67 g), WSC.HCl (1.74 g), HOBt (2.78 g) and DMF (20 ml) was stirred at 50° C. for 12 hr. The reaction mixture was cooled to room temperature, poured into a 1 N aqueous sodium hydroxide solution, and the mixture was extracted with ethyl acetate. The extract was washed with brine, and dried over anhydrous magnesium sulfate. The solvent was evaporated in vacuo. The residue was subjected to silica gel column chromatography, and the fraction eluted with ethyl acetate-methanol (1:0 to 5.5:1) was concentrated in vacuo to give the desired product (1.16 g).

MS (ESI+, m/e) 586 (M+1)

### Reference Example 536

### tert-Butyl (3R)-3-benzyl-4-({1-[4-hydroxy-1-(methoxycarbonyl)-4-(methoxymethyl)piperidin-3-yl]-5-phenyl-1H-imidazol-4-yl}carbonyl)piperazine-1-carboxylate and tert-butyl (3R)-3-benzyl-4-({1-[1-[(benzyloxy)carbonyl]-4-hydroxy-4-(methoxymethyl)piperidin-3-yl]-5-phenyl-1H-imidazol-4-yl}carbonyl)piperazine-1-carboxylate

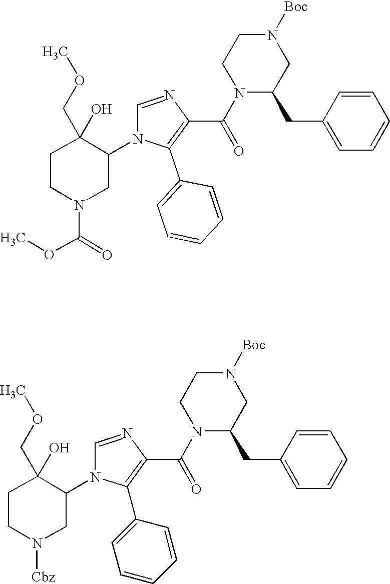

Benzyl 4-[4-(ethoxycarbonyl)-5-phenyl-1H-imidazol-1-yl]-1-oxa-6-azaspiro[2.5]octane-6-carboxylate (290 mg) was dissolved in methanol (5 ml), sodium methoxide (28% methanol solution, 0.4 ml) was added and the mixture was stirred at 50° C. for 12 hr. Water (5 ml) was added to the reaction mixture, and the mixture was stirred at 50° C. for additional 4 hr. The reaction mixture was concentrated in vacuo, the residue was suspended in ethanol again and the mixture was concentrated in vacuo. The residue was vacuum dried, suspended in DMF (10 ml), tert-butyl (3R)-3-benzylpiperazine-1-carboxylate (210 mg), WSC.HCl (182 mg) and HOBt (386 mg) were added, and the mixture was stirred at 60° C. for 3 hr. The reaction mixture was poured into saturated aqueous sodium bicarbonate solution, and the mixture was extracted with ethyl acetate. The extract was washed successively with water and brine, and dried over anhydrous sodium sulfate, and the solvent was evaporated in vacuo. The residue was subjected to silica gel column chromatography, and the fraction eluted with ethyl acetate-methanol (85:15) was concentrated in vacuo to give desired mixture (222 mg), each as an amorphous solid.

MS (ESI+, m/e) 648 (M+1), 724 (M+1)

### Reference Example 537

### tert-Butyl (3S)-3-[(4-acetylphenoxy)methyl]-4-([1-(3-morpholinophenyl)-5-phenyl-1H-imidazol-4-yl]carbonyl}piperazine-1-carboxylate

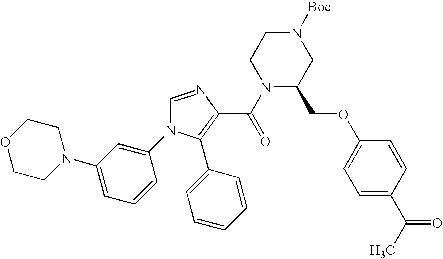

A solution of 1-(3-morpholinophenyl)-5-phenyl-1H-imidazole-4-carboxylic acid (419 mg), tert-butyl (3S)-3-([4-(1-hydroxyethyl)phenoxy]methyl}piperazine-1-carboxylate (404 mg), WSC.HCl (253 mg), HOBt (184 mg) and DMF (5 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-methanol (1:0 to 9:1) was concentrated in vacuo to give tert-butyl (3S)-3-{[4-(1-hydroxyethyl)phenoxy]methyl}-4-{[1-(3-morpholinophenyl)-5-phenyl-1H-imidazol-4-yl]carbonyl}piperazine-1-carboxylate (565 mg) as an amorphous solid. A 450 mg portion thereof was dissolved in dichloromethane (5 ml), and the solution was added to a suspension of Dess-Martin reagent (343 mg) in dichloromethane (5 ml). After stirring at room temperature for 2 hr, the mixture was poured into 6% aqueous sodium bicarbonate solution, and the mixture was extracted with ethyl acetate. The extract was washed with brine, and dried over anhydrous magnesium sulfate. The solvent was evaporated in vacuo. The residue was subjected to basic silica gel column chromatography, and the fraction eluted with ethyl acetate-hexane (1:9 to 3:7) was concentrated in vacuo to give the desired product (320 mg) as an amorphous solid.

MS (ESI+, m/e) 666 (M+1)

### Reference Example 538

### 4-[((2R)-4-Benzyl-1-{[1-(3-morpholinophenyl)-5-phenyl-1H-imidazol-4-yl]carbonyl}piperazin-2-yl)methyl]benzoic acid

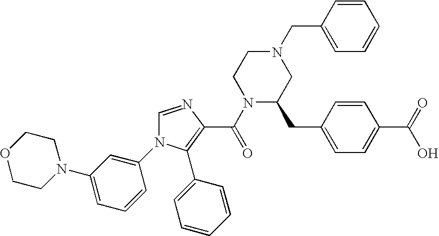

1-(3-Morpholinophenyl)-5-phenyl-1H-imidazole-4-carboxylic acid (349 mg), ethyl 4-{[(2R)-4-benzylpiperazin-2-yl]methyl}benzoate (338 mg), WSC.HCl (210 mg) and HOBt (160 mg) were dissolved in DMF (5 ml) and, after stirring at room temperature for 15 hr, and the solution was poured into saturated aqueous sodium bicarbonate solution, and extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:9 to 3:7) was concentrated in vacuo to give ethyl 4-[((2R)-4-benzyl-1-{[1-(3-morpholinophenyl)-5-phenyl-1H-imidazol-4-yl]carbonyl}piperazin-2-yl)methyl]benzoate (440 mg) as an amorphous solid. A 435 mg portion thereof was dissolved in ethanol (5 ml), pulverized potassium hydroxide (110 mg) was added, and the mixture was stirred at 80° C. for 1 hr. The reaction mixture was concentrated in vacuo, the residue was adjusted to pH 6-7 with a 10% aqueous citric acid solutiod, and the mixture was extracted with chloroform. The extract was washed with brine, and dried over anhydrous magnesium sulfate. The solvent was evaporated in vacuo to give the desired product as crystals.

MS (ESI+, m/e) 642 (M+1)

### Reference Example 539

### tert-Butyl 3-(2-methoxy-2-oxoethyl)-4-[(5-methyl-1,2-diphenyl-1H-pyrrol-3-yl)carbonyl]piperazine-1-carboxylate

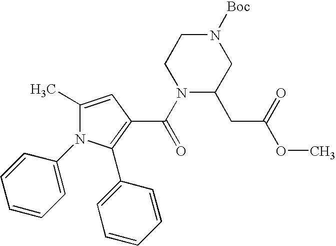

5-Methyl-1,2-diphenyl-1H-pyrrole-3-carboxylic acid (2.76 g) was suspended in THF (50 ml), and oxalyl chloride (1.52 g) and DMF (25 μl) were added. After stirring at room temperature for 2 hr, the reaction mixture was concentrated in vacuo, and the residue was dissolved in THF (25 ml), and the solution was added to a solution of tert-butyl 3-(2-methoxy-2-oxoethyl)piperazine-1-carboxylate (2.57 g) and pyridine (0.94 g) in THF (75 ml). After stirring at room temperature for 15 hr, the reaction mixture was poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo. The insoluble material was filtered off, and the residue was washed with ethyl acetate. The filtrate was concentrated in vacuo, and the residue was subjected to basic silica gel column chromatography, and the fraction eluted with ethyl acetate-hexane (1:2 to 1:1) was concentrated in vacuo, and the crystals were collected by filtration to give the desired product (3.27 g).

MS (ESI+, m/e) 518 (M+1)

### Reference Example 540

### Methyl {4-benzyl-1-[(5-methyl-1,2-diphenyl-1H-pyrrol-3-yl)carbonyl]piperazin-2-yl}acetate

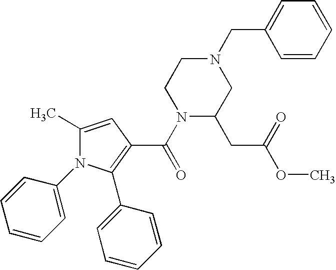

5-Methyl-1,2-diphenyl-1H-pyrrole-3-carboxylic acid (5.55 g) was suspended in THF (100 ml), and oxalyl chloride (3.05 g) and DMF (50 μl) were added. After stirring at room temperature for 2 hr, the reaction mixture was concentrated in vacuo. The residue was dissolved in THF (30 ml), and the solution was added to a solution of methyl (4-benzylpiperazin-2-yl)acetate (4.97 g) and triethylamine (2.43 g) in THF (75 ml). After stirring at room temperature for 2 hr, the reaction mixture was poured into saturated aqueous sodium bicarbonate solution, and the mixture was extracted with ethyl acetate. The extract was washed successively with water, 1% aqueous potassium carbonate solution, water and brine, and dried over anhydrous magnesium sulfate. The solvent was evaporated in vacuo. The residue was subjected to silica gel column chromatography, and the fraction eluted with ethyl acetate-hexane (1.5:1) was concentrated in vacuo to give the desired product (9.49 g) as an amorphous solid.

MS (ESI+, m/e) 508 (M+1)

### Reference Example 541

### ((2S,6R)-4,6-Dibenzyl-1-{[1-(3-bromophenyl)-5-methyl-2-phenyl-1H-pyrrol-3-yl]carbonyl)piperazin-2-yl)methanol

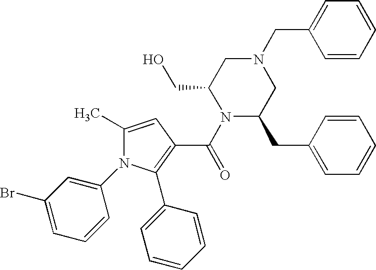

1-(3-Bromophenyl)-5-methyl-2-phenyl-1H-pyrrole-3-carboxylic acid (345 mg) was suspended in dichloromethane (10 ml), the suspension was ice-cooled, and DMF (2 drops) and oxalyl chloride (148 mg) were added. After stirring at room temperature for 1 hr, the reaction mixture was concentrated in vacuo. The residue was diluted with toluene, and concentrated again in vacuo. The residue was dissolved in dichloromethane (5 ml), and the solution was added to a solution of [(2S,6R)-4,6-dibenzylpiperazin-2-yl]methanol (261 mg) and triethylamine (147 mg) in dichloromethane (5 ml). After stirring at room temperature for 1 hr, the mixture was poured into saturated aqueous sodium bicarbonate solution, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate was concentrated in vacuo to give the desired product (380 mg) as an amorphous solid.

MS (ESI+, m/e) 635 (M+1)

In the same manner as in Reference Example 541, the following compounds (Reference Examples 542 and 543) were obtained:

### Reference Example 542

### (2R,6R)-2,4-Dibenzyl-1-{[1-(3-bromophenyl)-5-methyl-2-phenyl-1H-pyrrol-3-yl]carbonyl}-6-methylpiperazine

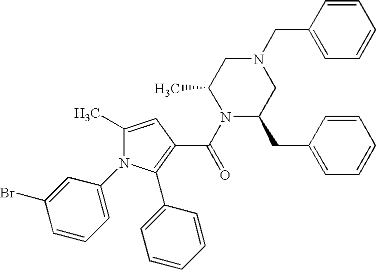

MS (ESI+, m/e) 619 (M+1)

### Reference Example 543

### tert-Butyl (3R)-3-benzyl-4-{[5-cyclohexyl-1-(3-morpholinophenyl)-1H-pyrazol-4-yl]carbonyl}piperazine-1-carboxylate

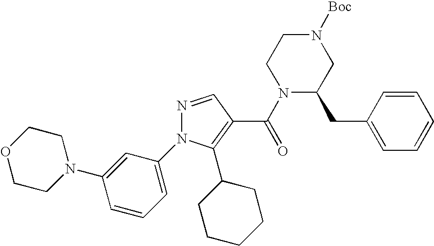

MS (ESI+, m/e) 614 (M+1)

### Reference Example 544

### 4-{3-[4-({(2R)-4-Benzyl-2-[4-(1H-tetrazol-5-yl)benzyl]piperazin-1-yl}carbonyl)-5-phenyl-1H-imidazol-1-yl]phenyl}morpholine

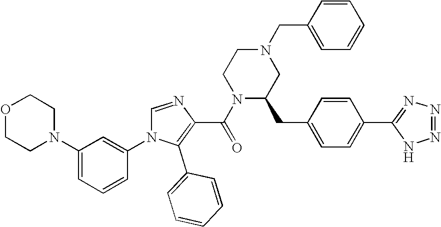

4-[((2R)-4-Benzyl-1-{[1-(3-morpholinophenyl)-5-phenyl-1H-imidazol-4-yl]carbonyl}piperazin-2-yl)methyl]benzonitrile (311 mg) was dissolved in toluene (5 ml), trimethylsilylazide (58 mg) and di-n-butyltin oxide (12.5 mg) were added and the mixture was heated under reflux for 8 hr. The solvent was evaporated in vacuo, the residue was subjected to silica gel column chromatography, and the fraction eluted with ethyl acetate-methanol (1:0 to 9:1) was concentrated in vacuo to give the desired product (205 mg) as an amorphous solid.

MS (ESI,+, m/e) 666 (M+1)

### Reference Example 545

### N-(Aminosulfonyl)-4-[((2R)-4-benzyl-1-{[1-(3-morpholinophenyl)-5-phenyl-1H-imidazol-4-yl]carbonyl}piperazin-2-yl)methyl]benzamide

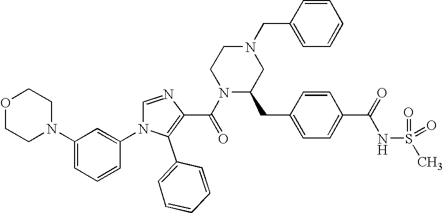

4-[((2R)-4-Benzyl-1-{[1-(3-morpholinophenyl)-5-phenyl-1H-imidazol-4-yl]carbonyl}piperazin-2-ylmethyl)benzoic acid (200 mg) was dissolved in DMF (5 ml), DSC (397 mg) was added and the mixture was stirred at room temperature for 2 hr. Methylsulfonamide (147 mg) and DBU (236 mg) were added thereto and the mixture was stirred at 110° C. for 15 hr. The reaction mixture was poured into water, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-methanol (1:0 to 9:1) was concentrated in vacuo to give the desired product (100 mg) as an amorphous solid.

MS (ESI+, m/e) 719 (M+1)

### Reference Example 546

### tert-Butyl (3R)-4-{[1-(3-morpholinophenyl)-5-phenyl-1H-imidazol-4-yl]carbonyl}-3-[4-(5-oxo-4,5-dihydro-1,2,4-oxadiazol-3-yl)benzyl]piperazine-1-carboxylate

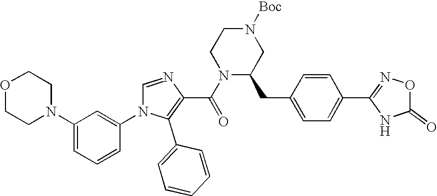

tert-Butyl (3R)-3-(4-cyanobenzyl)-4-{[1-(3-morpholinophenyl)-5-phenyl-1H-imidazol-4-yl]carbonyl}piperazine-1-carboxylate (180 mg) was dissolved in DMSO (3 ml), hydroxylamine hydrochloride (40 mg) and sodium bicarbonate (48 mg) were added and the mixture was stirred at 90° C. for 15 hr. The reaction mixture was poured into water, and the crystals were collected by filtration, and washed with brine. This was dissolved in ethyl acetate and the solution was dried over anhydrous sodium sulfate. The solvent was evaporated in vacuo and the residue was dissolved in THF (2 ml), DBU (55 mg) and CDI (59 mg) were added, and the mixture was stirred at 50° C. for 15 hr. The reaction mixture was poured into brine, and the mixture was extracted with ethyl acetate. The extract was washed with brine, and dried over anhydrous sodium sulfate. The solvent was evaporated in vacuo. The residue was subjected to silica gel column chromatography, and the fraction eluted with ethyl acetate-methanol (9:1) was concentrated in vacuo to give the desired product (136 mg) as an amorphous solid.

MS (ESI+, m/e) 692 (M+1)

### Reference Example 547

### 2-{4-[((2R)-4-Benzyl-1-{[1-(2,3-dihydro-1H-inden-2-yl)-5-phenyl-1H-imidazol-4-yl]carbonyl}piperazin-2-yl)methyl]phenyl}propan-2-ol

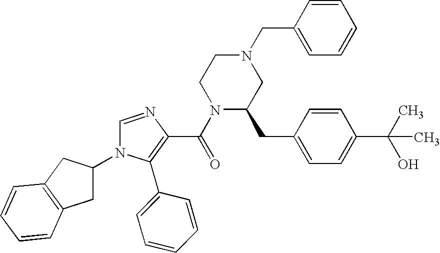

Ethyl 4-[((2R)-4-benzyl-1-{[1-(2,3-dihydro-1H-inden-2-yl)-5-phenyl-1H-imidazol-4-yl]carbonyl}piperazin-2-yl)methyl]benzoate (250 mg) was dissolved in toluene (3 ml) and the mixture was cooled to −40° C. Methylmagnesium bromide (1 M THF solution, 2 ml) was added and the mixture was stirred at the same temperature for 30 min and at 80° C. for 1 hr. A saturated aqueous ammonium chloride solution (5 ml) was added to the reaction mixture, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate was concentrated in vacuo to give the desired product (42 mg) as an amorphous solid.

MS (ESI+, m/e) 611 (M+1)

### Reference Example 548

### tert-Butyl (3S)-4-[(2-ethoxy-1,5-diphenyl-1H-imidazol-4-yl)carbonyl]-3-[(phenylsulfinyl)methyl]piperazine-1-carboxylate

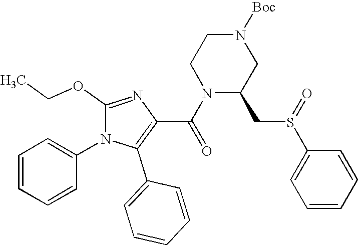

tert-Butyl (3S)-4-[(2-ethoxy-1,5-diphenyl-1H-imidazol-4-yl)carbonyl]-3-[(phenylthio)methyl]piperazine-1-carboxylate (360 mg) was dissolved in THF (5 ml) and the mixture was ice-cooled. A solution of m-chloroperbenzoic acid (148 mg) in THF (1 ml) was added, and the mixture was stirred at the same temperature for 30 min. The mixture was neutralized with saturated aqueous sodium bicarbonate solution, and the mixture was extracted with ethyl acetate. The extract was dried over anhydrous sodium sulfate, and the solvent was evaporated in vacuo. The residue was subjected to silica gel column chromatography, and the fraction eluted with ethyl acetate was concentrated in vacuo to give the desired product (230 mg) as an amorphous solid.

MS (ESI+, m/e) 615 (M+1)

### Reference Example 549

### tert-Butyl (3S)-4-[(2-ethoxy-1,5-diphenyl-1H-imidazol-4-yl)carbonyl]-3-[(phenylsulfonyl)methyl]piperazine-1-carboxylate

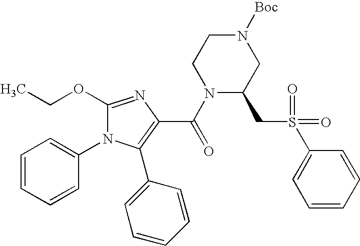

tert-Butyl (3S)-4-[(2-ethoxy-1,5-diphenyl-1H-imidazol-4-yl)carbonyl]-3-[(phenylthio)methyl]piperazine-1-carboxylate (360 mg) was dissolved in THF (5 ml) and the mixture was ice-cooled. A solution of m-chloroperbenzoic acid (296 mg) in THF (1 ml) was added, and the mixture was stirred at the same temperature for 30 min. The mixture was neutralized with saturated aqueous sodium bicarbonate solution, and the mixture was extracted with ethyl acetate. The extract was dried over anhydrous sodium sulfate, and the solvent was evaporated in vacuo. The residue was subjected to silica gel column chromatography, and the fraction eluted with ethyl acetate was concentrated in vacuo to give the desired product (270 mg) as an amorphous solid.

MS (ESI+, m/e) 631 (M+1)

### Reference Example 550

### Ethyl [4-({(2R)-4-benzyl-1-[(5-methyl-1,2-diphenyl-1H-pyrrol-3-yl)carbonyl]piperazin-2-yl}methyl)phenoxy]acetate

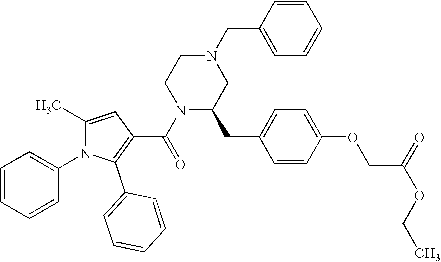

To a solution of 4-({(2R)-4-benzyl-1-[(5-methyl-1,2-diphenyl-1H-pyrrol-3-yl)carbonyl]piperazin-2-yl}methyl)phenol (1.0 g), ethyl bromoacetate (340 mg) and DMF (10 ml) was added potassium carbonate (770 mg) and the mixture was stirred at 80° C. for 2 hr. The mixture was poured into ice-cooled water, and extracted with ethyl acetate. The extract was dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (1.1 g) as an amorphous solid.

MS (ESI+, m/e) 628 (M+1)

### Reference Example 551

### [4-({(2R)-4-Benzyl-1-[(5-methyl-1,2-diphenyl-1H-pyrrol-3-yl)carbonyl]piperazin-2-yl}methyl)phenoxy]acetic acid

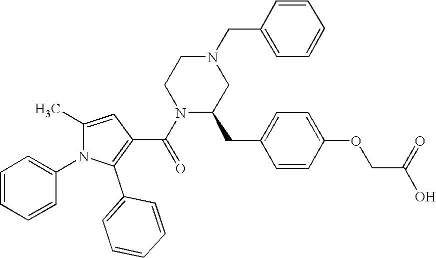

Ethyl [4-({(2R)-4-benzyl-1-[(5-methyl-1,2-diphenyl-1H-pyrrol-3-yl)carbonyl]piperazin-2-yl}methyl)phenoxy]acetate (500 mg) was dissolved in ethanol (3 ml), and a 2 N aqueous sodium hydroxide solution (2 ml) was added. After stirring at room temperature for 2 hr, the solvent was evaporated in vacuo. The residual aqueous solution was neutralized with 2 N hydrochloric acid, and the mixture was extracted with ethyl acetate. The extract was washed with brine, and dried over anhydrous sodium sulfate, and the solvent was evaporated in vacuo to give the desired product (480 mg).

MS (ESI+, m/e) 600 (M+1)

### Reference Example 552

### 2-[4-({(2R)-4-Benzyl-1-[(5-methyl-1,2-diphenyl-1H-pyrrol-3-yl)carbonyl]piperazin-2-yl}methyl)phenoxy]-N-(methylsulfonyl)acetamide

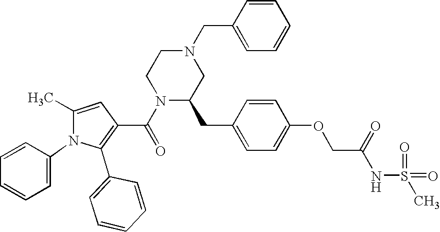

[4-({4-Benzyl-1-[(5-methyl-1,2-diphenyl-1H-pyrrol-3-yl)carbonyl]piperazin-2-yl}methyl)phenoxy]acetic acid (209 mg) was dissolved in DMF (5 ml), DSC (446 mg) was added and the mixture was stirred at room temperature for 1 hr. Methylsulfonamide (165 mg) and DBU (264 mg) were added and the mixture was stirred at 90° C. for 3 hr. The reaction mixture was poured into water, and the crystals were collected by filtration and washed with water and a small amount of ethyl acetate. This was subjected to silica gel column chromatography, and the fraction eluted with ethyl acetate-methanol (9:1) was concentrated in vacuo to give the desired product (390 mg) as an amorphous solid.

MS (ESI+, m/e) 677 (M+1)

### Reference Example 553

### tert-Butyl (3R)-3-(4-hydroxybenzyl)-4-[(5-methyl-1,2-diphenyl-1H-pyrrol-3-yl)carbonyl]piperazine-1-carboxylate

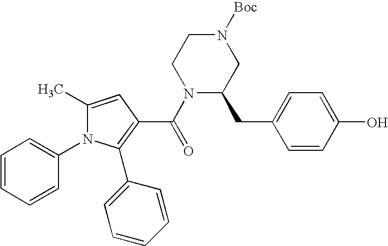

4-({(2R)-4-Benzyl-1-[(5-methyl-1,2-diphenyl-1H-pyrrol-3-yl)carbonyl]piperazin-2-yl}methyl)phenol (1.0 g) was dissolved in methanol (10 ml), 20% palladium hydroxide on carbon (containing 50% water) (100 mg) was added, and a catalytic hydrogenation was performed at room temperature and atmospheric pressure for 15 hr. The catalyst was filtered off, and the filtrate was concentrated in vacuo to give 4-({(2R)-1-[(5-methyl-1,2-diphenyl-1H-pyrrol-3-yl)carbonyl]piperazin-2-yl}methyl)phenol (850 mg). A 800 mg portion thereof was dissolved in THF (10 ml) and the solution was ice-cooled. N,N-Diisopropylethylamine (457 mg) and di-tert-butyl dicarbonate (390 mg) were added. After stirring at room temperature for 12 hr, the mixture was poured into water, and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (1.0 g) as an amorphous solid.

MS (ESI+, m/e) 552 (M+1)

### Reference Example 554

### tert-Butyl (3R)-3-[4-(cyanomethoxy)benzyl]-4-[(5-methyl-1,2-diphenyl-1H-pyrrol-3-yl)carbonyl]piperazine-1-carboxylate

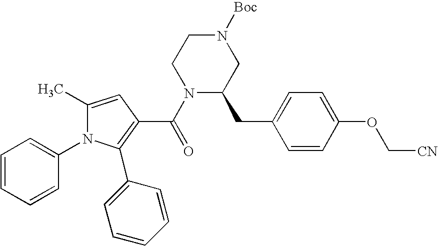

To a solution of tert-butyl (3R)-3-(4-hydroxybenzyl)-4-[(5-methyl-1,2-diphenyl-1H-pyrrol-3-yl)carbonyl]piperazine-1-carboxylate (490 mg), chloroacetonitrile (200 mg) and acetone (10 ml) was added potassium carbonate (610 mg). After stirring at 80° C. for 12 hr, the solvent was evaporated in vacuo. The residue was suspended in ethyl acetate, and the mixture was washed with brine, and dried over anhydrous sodium sulfate. The solvent was evaporated in vacuo, the residue was subjected to basic silica gel column chromatography, and the fraction eluted with ethyl acetate-hexane (1:1) was concentrated in vacuo to give the desired product (430 mg) as an amorphous solid.

MS (ESI+, m/e) 591 (M+1)

### Reference Example 555

### tert-Butyl (3R)-4-[(5-methyl-1,2-diphenyl-1H-pyrrol-3-yl)carbonyl]-3-{4-[(5-oxo-4,5-dihydro-1,2,4-oxadiazol-3-yl)methoxy]benzyl}piperazine-1-carboxylate

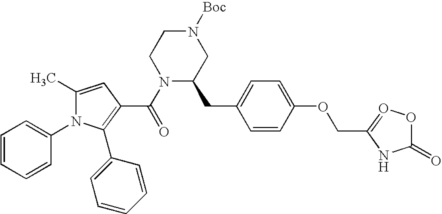

tert-Butyl (3R)-3-[4-(cyanomethoxy)benzyl]-4-[(5-methyl-1,2-diphenyl-1H-pyrrol-3-yl)carbonyl]piperazine-1-carboxylate (300 mg) was dissolved in DMSO (3 ml), hydroxylamine hydrochloride (71 mg) and sodium bicarbonate (85 mg) were added and the mixture was stirred at room temperature for 12 hr. The reaction mixture was poured into water, and the precipitated crystals were collected by filtration and washed with brine. This was dissolved in ethyl acetate and the solution was dried over anhydrous sodium sulfate. The solvent was evaporated in vacuo and the residue was dissolved in THF (5 ml). DBU (77 mg) and CDI (82 mg) were added, and the mixture was stirred at room temperature for 3 hr. The reaction mixture was poured into brine, and the mixture was extracted with ethyl acetate. The extract was washed with brine, and dried over anhydrous sodium sulfate. The solvent was evaporated in vacuo. The residue was subjected to silica gel column chromatography, and the fraction eluted with ethyl acetate-methanol (9:1) was concentrated in vacuo to give the desired product (280 mg) as an amorphous solid.

MS (ESI+, m/e) 650 (M+1)

### Reference Example 556

### [4-({(2R)-4-(tert-Butoxycarbonyl)-1-[(5-methyl-1,2-diphenyl-1H-pyrrol-3-yl)carbonyl]piperazin-2-yl}methyl)phenoxy]acetic acid

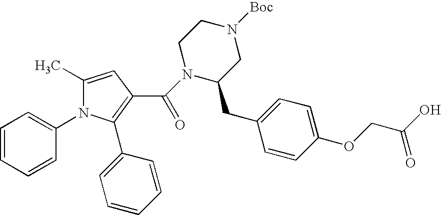

Ethyl (4-({(2R)-4-benzyl-1-[(5-methyl-1,2-diphenyl-1H-pyrrol-3-yl)carbonyl]piperazin-2-yl}methyl)phenoxy]acetate (450 mg) was dissolved in methanol (5 ml), 20% palladium hydroxide on carbon (containing 50% water) (100 mg) was added, and a catalytic hydrogenation was performed at room temperature and atmospheric pressure for 15 hr. The catalyst was filtered off, and the filtrate was concentrated in vacuo to give ethyl [4-({(2R)-1-[(5-methyl-1,2-diphenyl-1H-pyrrol-3-yl)carbonyl]piperazin-2-yl}methyl)phenoxy]acetate (381 mg). The total amount thereof was dissolved in THF (5 ml) and the mixture was ice-cooled. N,N-Diisopropylethylamine (184 mg) and di-tert-butyl dicarbonate (155 mg) were added. After stirring at room temperature for 12 hr, the mixture was poured into water, and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (1:1) was concentrated in vacuo to give ethyl [4-({(2R)-4-(tert-butoxycarbonyl)-1-[(5-methyl-1,2-diphenyl-1H-pyrrol-3-yl)carbonyl]piperazin-2-yl}methyl)phenoxy]acetate (450 mg). The total amount thereof was dissolved in ethanol (4 ml), and a 2 N aqueous sodium hydroxide solution (6 ml) was added. After stirring at room temperature for 3 hr, the mixture was poured into ice-water, and the mixture was neutralized by adding 2 N hydrochloric acid by small portions, and extracted with ethyl acetate. The extract was washed with brine, and dried over anhydrous sodium sulfate, and the solvent was evaporated in vacuo to give the desired product (380 mg).

MS (ESI+, m/e) 610 (M+1)

### Reference Example 557

### tert-Butyl (3R)-4-[(5-methyl-1,2-diphenyl-1H-pyrrol-3-yl)carbonyl]-3-(4-{2-[(5-methyl-2-oxo-1,3-dioxol-4-yl)methoxy]-2-oxoethoxy}benzyl)piperazine-1-carboxylate

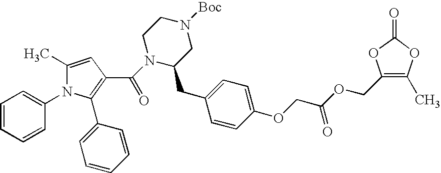

[4-({(2R)-4-(tert-Butoxycarbonyl)-1-[(5-methyl-1,2-diphenyl-1H-pyrrol-3-yl)carbonyl]piperazin-2-yl}methyl)phenoxy]acetic acid (360 mg) and 4-(hydroxymethyl)-5-methyl-1,3-dioxol-2-one (92 mg) were dissolved in DMA (4 ml) and the mixture was ice-cooled. p-Toluenesulfonyl chloride (135 mg), DMAP (14 mg) and potassium carbonate (106 mg) were added and the mixture was stirred at 0° C. for 5 hr and at room temperature for 12 hr. The reaction mixture was poured into an ice-cooled 5% aqueous citric acid solution, and the mixture was extracted with ethyl acetate. The extract was washed with brine, and dried over anhydrous sodium sulfate. The solvent was evaporated in vacuo. The residue was subjected to silica gel column chromatography, and the fraction eluted with ethyl acetate-hexane (1:1) was concentrated in vacuo to give the desired product (320 mg) as an amorphous solid.

MS (ESI+, m/e) 722 (M+1)

### Reference Example 558

### 4-[((2R)-4-Benzyl-1-{[1-(2,3-dihydro-1H-inden-2-yl)-5-phenyl-1H-imidazol-4-yl]carbonyl}piperazin-2-yl)methyl]benzoic acid

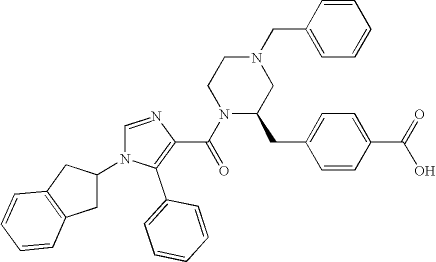

Ethyl 4-[((2R)-4-benzyl-1-{[1-(2,3-dihydro-1H-inden-2-yl)-5-phenyl-1H-imidazol-4-yl]carbonyl}piperazin-2-yl)methyl]benzoate (670 mg) was dissolved in ethanol (25 ml), potassium hydroxide (56 mg) was added, and the mixture was heated under reflux for 1 hr. The solvent was evaporated in vacuo and the residue was adjusted to pH 7 with a 10% aqueous citric acid solution. The liberated oil was extracted with chloroform, and the extract was washed with brine, and dried over anhydrous magnesium sulfate. The solvent was evaporated in vacuo to give the desired-product (160 mg) as an amorphous solid.

MS (ESI+, m/e) 597 (M+1)

### Reference Example 559

### tert-Butyl (3S)-3-[(1R)-3-tert-butoxy-1-hydroxy-3-oxopropyl]-4-[(5-methyl-1,2-diphenyl-1H-pyrrol-3-yl)carbonyl]piperazine-1-carboxylate and tert-butyl (3S)-3-[(1S)-3-tert-butoxy-1-hydroxy-3-oxopropyl]-4-[(5-methyl-1,2-diphenyl-1H-pyrrol-3-yl)carbonyl]piperazine-1-carboxylate

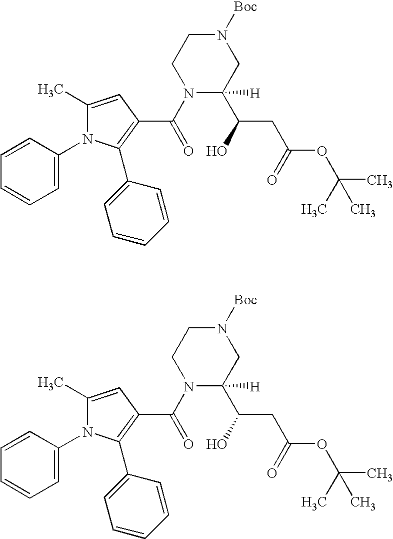

tert-Butyl (3S)-3-formyl-4-[(5-methyl-1,2-diphenyl-1H-pyrrol-3-yl)carbonyl]piperazine-1-carboxylate (300 mg) was dissolved in THF (4 ml), (2-tert-butoxy-2-oxoethyl)zinc bromide (0.5 M THF solution, 7 ml) was added, and the mixture was stirred at room temperature for 1 hr, and at 60° C. for 30 min. The reaction mixture was poured into water and extracted with ethyl acetate. The extract was dried over anhydrous sodium sulfate, and the solvent was evaporated in vacuo. The residue was subjected to, silica gel column chromatography, and a diastereomer was separated from the fraction eluted with ethyl acetate-hexane (1:1), and the fractions were each concentrated in vacuo to give the desired products (180 mg and 140 mg), each as an amorphous solid.

MS (ESI+, m/e) 590 (M+1)

MS (ESI+, m/e) 590 (M+1)

In the same manner as in Reference Example 559, the following compound (Reference Example 560) was obtained as a diastereomixture.

### Reference Example 560

### tert-Butyl (3S)-3-(3-ethoxy-1-hydroxy-3-oxopropyl)-4-[(5-methyl-1,2-diphenyl-1H-pyrrol-3-yl)carbonyl]piperazine-1-carboxylate

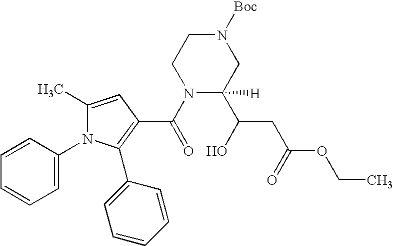

MS (ESI+, m/e) 562 (M+1)

### Reference Example 561

### 3-{(2S)-4-(tert-Butoxycarbonyl)-1-[(5-methyl-1,2-diphenyl-1H-pyrrol-3-yl)carbonyl]piperazin-2-yl}-3-hydroxypropionic acid

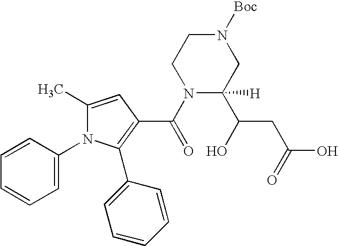

To a solution of tert-butyl (3S)-3-(3-ethoxy-1-hydroxy-3-oxopropyl)-4-[(5-methyl-1,2-diphenyl-1H-pyrrol-3-yl)carbonyl]piperazine-1-carboxylate (710 mg) in ethanol (2 ml) was added a 1 N aqueous sodium hydroxide solution (4 ml). After stirring at room temperature for 1 hr, the solvent was evaporated in vacuo. The residual aqueous solution was washed with ethyl acetate, and neutralized with a 10% aqueous citric acid solution. This was extracted with ethyl acetate, the extract was washed with brine, and dried over anhydrous sodium sulfate, and the solvent was evaporated in vacuo to give the desired product (670 mg).

MS (ESI+, m/e) 534 (M+1)

### Reference Example 562

### tert-Butyl (3S)-3-(3-amino-1-hydroxy-3-oxopropyl)-4-[(5-methyl-1,2-diphenyl-1H-pyrrol-3-yl)carbonyl]piperazine-1-carboxylate

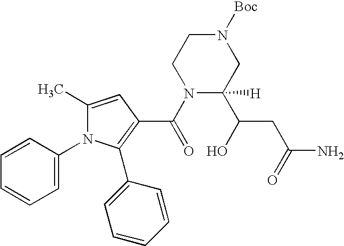

A solution of 3-((2S)-4-(tert-butoxycarbonyl)-1-[(5-methyl-1,2-diphenyl-1H-pyrrol-3-yl)carbonyl]piperazin-2-yl}-3-hydroxypropionic acid (670 mg), HOBt ammonium salt (258 mg), WSC.HCl (290 mg) and DMF (4 ml) was stirred at room temperature for 12 hr, and poured into saturated aqueous sodium bicarbonate solution, and the mixture was extracted with ethyl acetate-THF (1:1). The extract was washed with brine, and dried over anhydrous sodium sulfate, and the solvent was evaporated in vacuo. The residue was subjected to basic silica gel column chromatography, and the fraction eluted with ethyl acetate-methanol (4:1) was concentrated in vacuo to give the desired product (240 mg) as an amorphous solid.

MS (ESI+, m/e) 533 (M+1)

### Reference Example 563

### tert-Butyl (3S)-4-{[1-(2,3-dihydro-1H-inden-2-yl)-5-phenyl-1H-imidazol-4-yl]carbonyl}-3-isobutyrylpiperazine-1-carboxylate

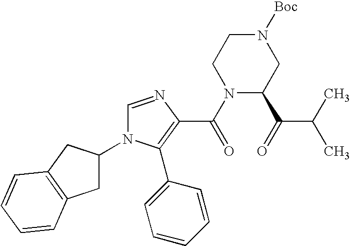

1-((2S)-1-{[1-(2,3-Dihydro-1H-inden-2-yl)-5-phenyl-1H-imidazol-4-yl]carbonyl}piperazin-2-yl)-2-methylpropan-1-one (compound of below-mentioned Example 312) (1.2 g) was dissolved in THF, and the mixture was ice-cooled. N,N-Diisopropylethylamine (700 mg) and di-tert-butyl dicarbonate (590 mg) were added. After stirring at room temperature for 12 hr, the mixture was poured into water, and extracted with ethyl acetate. The extract was washed with brine, and dried over anhydrous sodium sulfate, and the solvent was evaporated in vacuo. The residue was subjected to silica gel column chromatography, and the fraction eluted with ethyl, acetate was concentrated in vacuo to give the desired product (1.4 g) as an amorphous solid.

MS (ESI+, m/e) 543 (M+1)

### Reference Example 564

### tert-Butyl (3S)-4-{[1-(2,3-dihydro-1H-inden-2-yl)-5-phenyl-1H-imidazol-4-yl]carbonyl}-3-[(1RS)-1-hydroxy-2-methylpropyl]piperazine-1-carboxylate

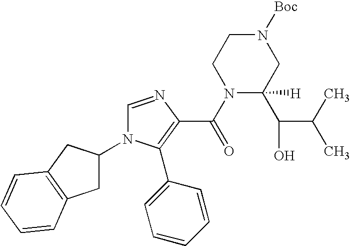

tert-Butyl (3S)-4-{[1-(2,3-dihydro-1H-inden-2-yl)-5-phenyl-1H-imidazol-4-yl]carbonyl}-3-isobutyrylpiperazine-1-carboxylate (170 mg) was dissolved in methanol (5 ml) and the mixture was ice-cooled. Sodium borohydride (59 mg) was added and the mixture was stirred at 0° C. for 15 min, and at room temperature for 30 min. Ice-water (5 ml) was added. The solvent was evaporated in vacuo, and the suspension was extracted with ethyl acetate. The extract was washed with brine, and dried over anhydrous sodium sulfate, and the solvent was evaporated in vacuo. The residue was subjected to basic silica gel column chromatography, and the fraction eluted with ethyl acetate was concentrated in vacuo to give the desired product (145 mg) as an amorphous solid.

MS (ESI+, m/e) 545 (M+1)

### Reference Example 565

### 1-{(2S)-4-Benzyl-1-[(5-methyl-1,2-diphenyl-1H-pyrrol-3-yl)carbonyl]piperazin-2-yl}-2-methylpropan-1-ol

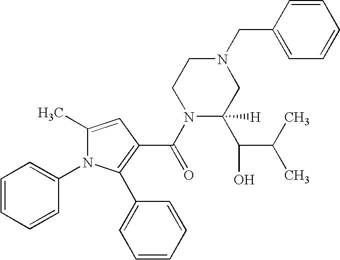

(2S)-4-Benzyl-1-[(5-methyl-1,2-diphenyl-1H-pyrrol-3-yl)carbonyl]piperazine-2-carbaldehyde (280 mg) was dissolved in THF (10 ml) and the mixture was cooled to −78° C. Isopropylmagnesium chloride. (2 M THF solution, 0.33 ml) was added and the mixture was stirred at the same temperature for 1 hr. A saturated aqueous ammonium chloride solution was added to the reaction mixture, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (3:7 to 7:3) was concentrated in vacuo to give the desired product (205 mg) as an amorphous solid.

MS (ESI+, m/e) 508 (M+1)

### Reference Example 566

### tert-Butyl (3S)-3-[(S)-cyclopropyl(hydroxy)methyl]-4-[(5-methyl-1,2-diphenyl-1H-pyrrol-3-yl)carbonyl]piperazine-1-carboxylate and tert-butyl (3S)-3-[(R)-cyclopropyl(hydroxy)methyl)-4-[(5-methyl-1,2-diphenyl-1H-pyrrol-3-yl)carbonyl]piperazine-1-carboxylate

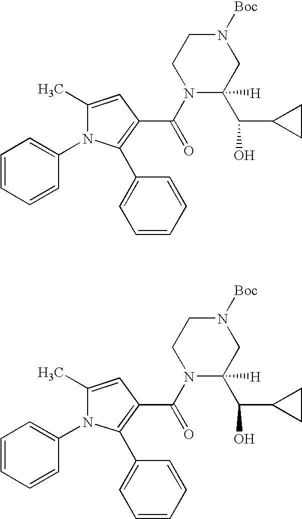

tert-Butyl (3S)-3-formyl-4-[(5-methyl-1,2-diphenyl-1H-pyrrol-3-yl)carbonyl]piperazine-1-carboxylate (240 mg) was dissolved in THF (10 ml) and the mixture was cooled to −78° C. Cyclopropylmagnesium bromide (0.5 M THF solution, 1.12 ml) was added and the mixture was stirred at the same temperature for 1 hr. A saturated aqueous ammonium chloride solution was added to the reaction mixture, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (1:9) was concentrated in vacuo to give the desired product (100 mg and 24 mg), each as an amorphous solid.

MS (ESI+, m/e) 516 (M+1)

MS (ESI+, m/e) 516 (M+1)

### Reference Example 567

tert-Butyl (3S)-3-(1-hydroxy-3-methylbutyl)-4-[(5-methyl-1,2-diphenyl-1H-pyrrol-3-yl)carbonyl]piperazine-1-carboxylate

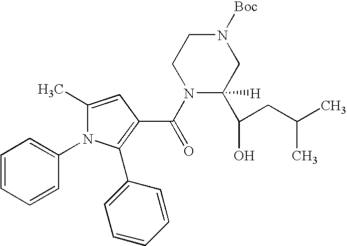

tert-Butyl (3S)-3-formyl-4-[(5-methyl-1,2-diphenyl-1H-pyrrol-3-yl)carbonyl]piperazine-1-carboxylate (170 mg) was dissolved in THF (2 ml) and the mixture was cooled to −40° C. Isobutylmagnesium bromide (1 M THF solution, 1.6 ml) was added at the same temperature, and the mixture was stirred for 30 min. A saturated aqueous ammonium chloride solution was added to the reaction mixture, and the mixture was extracted with ethyl acetate. The extract was washed with brine, and dried over anhydrous sodium sulfate. The solvent was evaporated in vacuo. The residue was subjected to basic silica gel column chromatography, and the fraction eluted with ethyl acetate was concentrated in vacuo to give the desired product (60 mg) as an amorphous solid.

MS (ESI+, m/e) 532 (M+1)

### Reference Example 568

### tert-Butyl (3S)-3-[(R)-hydroxy(phenyl)methyl]-4-[(5-methyl-1,2-diphenyl-1H-pyrrol-3-yl)carbonyl]piperazine-1-carboxylate and tert-butyl (3S)-3-[(S)-hydroxy(phenyl)methyl]-4-[(5-methyl-1,2-diphenyl-1H-pyrrol-3-yl)carbonyl]piperazine-1-carboxylate

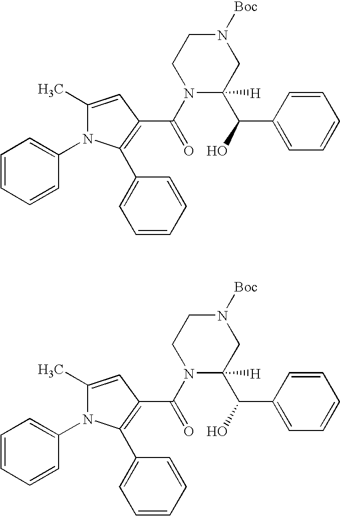

tert-Butyl (3S)-3-formyl-4-[(5-methyl-1,2-diphenyl-1H-pyrrol-3-yl)carbonyl]piperazine-1-carboxylate (1.00 g) was dissolved in THF (50 ml), and the mixture was cooled to −78° C. Phenylmagnesium bromide (1 M THF solution, 3.5 ml) was added and the mixture was stirred at the same temperature for 2 hr. A saturated aqueous ammonium chloride solution was added to the reaction mixture, and the mixture was extracted with ethyl acetate. The extract was washed with brine, and dried over anhydrous magnesium sulfate, and concentrated in vacuo. The residue was subjected to basic silica gel column chromatography, and the fraction eluted with ethyl acetate was concentrated in vacuo to give tert-butyl (3S)-3-[(R)-hydroxy(phenyl)methyl]-4-[(5-methyl-1,2-diphenyl-1H-pyrrol-3-yl)carbonyl]piperazine-1-carboxylate (85 mg) as an amorphous solid, and tert-butyl (3S)-3-[(S)-hydroxy(phenyl)methyl]-4-[(5-methyl-1,2-diphenyl-1H-pyrrol-3-yl)carbonyl]piperazine-1-carboxylate (300 mg) as an amorphous solid.

MS (ESI+, m/e) 552 (A+1)

MS (ESI+, m/e) 552 (M+1)

In the same manner as in Reference Example 568, the following compound (Reference Example 569) was obtained.

### Reference Example 569

### (1S)-1-{(2S)-4-Benzyl-1-[(5-methyl-1,2-diphenyl-1H-pyrrol-3-yl)carbonyl]piperazin-2-yl}-2-phenylethanol and (1R)-1-{(2S)-4-benzyl-1-[(5-methyl-1,2-diphenyl-1H-pyrrol-3-yl)carbonyl]piperazin-2-yl}-2-phenylethanol

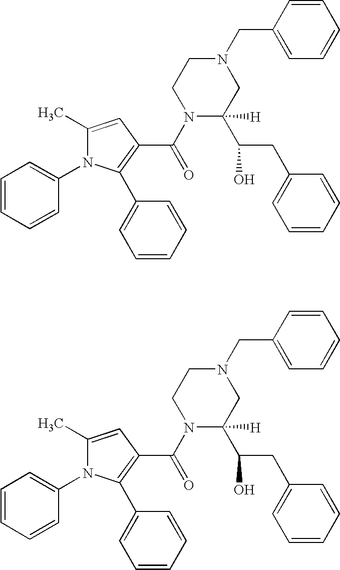

MS (ESI+, m/e) 556 (M+1)

MS (ESI+, m/e) 556 (M+1)

### Reference Example 570

### tert-Butyl (3R)-4-[(5-methyl-1,2-diphenyl-1H-pyrrol-3-yl)carbonyl]-3-[(E)-2-phenylvinyl]piperazine-1-carboxylate

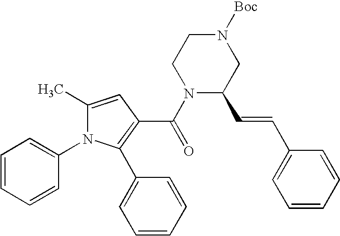

Diethyl benzylphosphonate was dissolved in THF (20 ml) and the mixture was cooled to −78° C. Butyllithium (1.6M THF solution, 2.6 ml) was added and the mixture was stirred at the same temperature for 30 min. The reaction mixture was cooled to 0° C., and a solution of tert-butyl (3S)-3-formyl-4-[(5-methyl-1,2-diphenyl-1H-pyrrol-3-yl)carbonyl]piperazine-1-carboxylate (1000 mg) in THF (10 ml) was added and, after stirring at −78° C. for 1 hr, and the mixture was stirred for additional 4 hr while allowing the mixture to warm to room temperature. A saturated aqueous ammonium chloride solution was added to the reaction mixture, and the mixture was extracted with ethyl acetate. The extract was washed with brine, dried over anhydrous magnesium sulfate, and concentrated in vacuo. The residue was subjected to silica gel column chromatography, and the fraction eluted with hexane-ethyl acetate (3:7) was concentrated in vacuo to give the desired product (374 mg) as an amorphous solid.

MS (ESI+, m/e) 548 (M+1)

### Reference Example 571

### tert-Butyl 3-(cyanomethyl)-4-[(5-methyl-1,2-diphenyl-1H-pyrrol-3-yl)carbonyl]piperazine-1-carboxylate

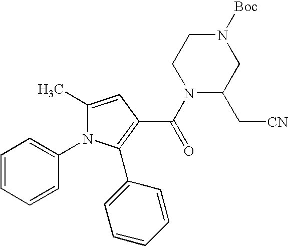

Potassium tert-butoxide (474 mg) was dissolved in DME (5 ml), p-toluenesulfonylmethylisocyanide (454 mg) was added at −78° C., and the mixture was stirred for 10 min. A solution of tert-butyl (3S)-3-formyl-4-[(5-methyl-1,2-diphenyl-1H-pyrrol-3-yl)carbonyl]piperazine-1-carboxylate (1.00 g) in DME (20 ml) was added dropwise, and the mixture was stirred at the same temperature for 30 min. After stirring the reaction mixture at room temperature for additional 10 min, methanol (25 ml) was added. After heating under reflux for 1 hr, the mixture was concentrated in vacuo. The residue was partitioned between ethyl acetate and 1 N hydrochloric acid, and the organic layer was washed successively with saturated aqueous sodium bicarbonate solution and brine, and dried over anhydrous sodium sulfate, and the solvent was evaporated in vacuo. The residue was subjected to silica gel column chromatography, and the fraction eluted with ethyl acetate-hexane (1:1.5) was concentrated in vacuo to give the desired product (320 mg) as an amorphous solid.

MS (ESI+, m/e) 485 (M+1)

### Reference Example 572

### tert-Butyl (3R)-3-benzyl-4-{[1-(3-hydroxyphenyl)-5-phenyl-1H-imidazol-4-yl]carbonyl}piperazine-1-carboxylate

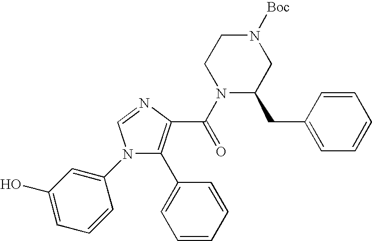

tert-Butyl (3R)-3-benzyl-4-[(5-phenyl-1-{3-[benzyloxy]phenyl}-1H-imidazol-4-yl)carbonyl]piperazine-1-carboxylate (720 mg) was dissolved in methanol (45 ml), 10% palladium on carbon (containing 50% water) (200 mg) was added, and a catalytic hydrogenation was performed at room temperature and atmospheric pressure for 12 hr. The catalyst was filtered off, and the filtrate was concentrated in vacuo. The residue was suspended in ethyl acetate, and the mixture was dried over anhydrous sodium sulfate, and the solvent was evaporated in vacuo. The residue was subjected to silica gel column chromatography, and the fraction eluted with ethyl acetate was concentrated in vacuo to give the desired product (599 mg) as an amorphous solid.

MS (ESI+, m/e) 539 (M+1)

### Reference Example 573

### tert-Butyl (3R)-3-benzyl-4-[(1-{3-[(1,1-dioxidotetrahydro-2H-thiopyran-4-yl)oxy]phenyl}-5-phenyl-1H-imidazol-4-yl)carbonyl]piperazine-1-carboxylate

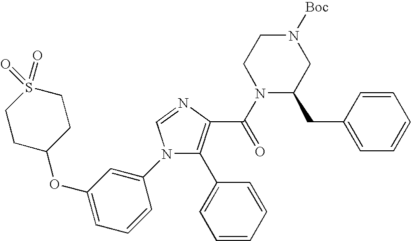

tert-Butyl (3R)-4-{[1-(3-hydroxyphenyl)-5-phenyl-1H-imidazol-4-yl]carbonyl}-3-benzylpiperazine-1-carboxylate (200 mg) was dissolved in DMF (5 ml), and potassium carbonate (283 mg) was added. After stirring at 80° C. for 12 hr, the reaction mixture was poured into water, and the mixture was extracted with ethyl acetate. The extract was washed with brine, and dried over anhydrous sodium sulfate, and the solvent was evaporated in vacuo. The residue was subjected to silica gel column chromatography, and the fraction eluted with ethyl acetate was concentrated in vacuo to give the desired product (210 mg) as an amorphous solid.

MS (ESI+, m/e) 671 (M+1)

### Reference Example 574

### tert-Butyl (3R)-3-benzyl-4-({1-[(1R)-1-benzyl-2-methoxyethyl]-5-phenyl-1H-imidazol-4-yl}carbonyl)piperazine-1-carboxylate

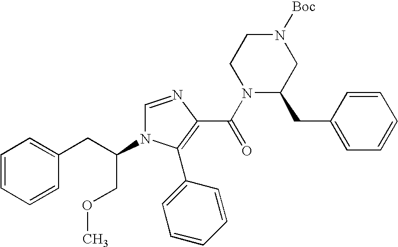

A mixture of tert-butyl (3R)-3-benzyl-4-({1-[(1R)-1-benzyl-2-hydroxyethyl]-5-phenyl-1H-imidazol-4-yl)carbonyl)piperazine-1-carboxylate (218 mg), sodium hydride (60% in oil) (25 mg) and THF (4.0 ml) was stirred at room temperature for 1 hr and ice-cooled. Methyl iodide (80 μl) was added to the reaction mixture and, after stirring at room temperature for additional 14 hr, the mixture was poured into saturated aqueous sodium bicarbonate solution, and extracted lo with ethyl acetate. The extract was washed with brine, and dried over anhydrous magnesium sulfate. The solvent was evaporated in vacuo. The residue was subjected to basic silica gel column chromatography, and the fraction eluted with ethyl acetate-methanol (1:0 to 9:1) was concentrated in vacuo to give the desired product (177 mg).

MS (ESI+, m/e) 595 (M+1)

### Reference Example 575

### tert-Butyl (3R)-3-benzyl-4-{[1-(4-oxotetrahydro-2H-pyran-3-yl)-5-phenyl-1H-imidazol-4-yl]carbonyl}piperazine-1-carboxylate

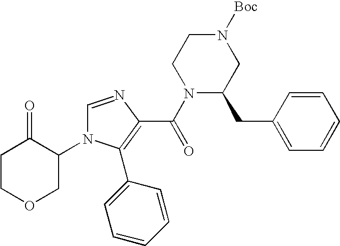

tert-Butyl (3R)-3-benzyl-4-([5-phenyl-1-(trans-4-hydroxytetrahydro-2H-pyran-3-yl)-1H-imidazol-4-yl]carbonyl}piperazine-1-carboxylate (440 mg) was dissolved in dichloromethane (5 ml), a solution of Dess-Martin reagent (410 mg) in dichloromethane (5 ml) was added and the mixture was stirred at room temperature for 3 hr. The reaction mixture was poured into a 10% aqueous sodium thiosulfate solution and the mixture was stirred for 30 min. After partitioning, the organic layer was washed with saturated aqueous sodium bicarbonate solution and brine and dried over anhydrous sodium sulfate. The solvent was evaporated in vacuo. The residue was subjected to silica gel column chromatography, and the fraction eluted with ethyl acetate was concentrated in vacuo to give the desired product (430 mg) as an amorphous solid.

MS (ESI+, m/e) 545 (M+1)

### Reference Example 576

### tert-Butyl (3R)-3-benzyl-4-{[1-(1,6-dioxaspiro[2.5]-4-octyl)-5-phenyl-1H-imidazol-4-yl]carbonyl}piperazine-1-carboxylate

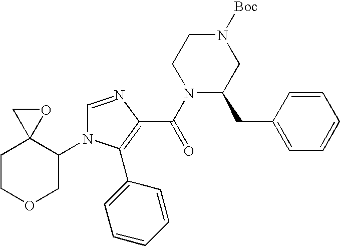

Trimethylsulfoxonium iodide (193 mg) was suspended in DMSO (4 ml), sodium hydride (6.0% in oil) (35 mg) was added, and the mixture was stirred at room temperature for 30 min. A solution of tert-butyl (3R)-3-benzyl-4-{[1-(4-oxotetrahydro-2H-pyran-3-yl)-5-phenyl-1H-imidazol-4-yl]carbonyl}piperazine-1-carboxylate (400 mg) in DMSO (8 ml) was added thereto and the mixture was stirred at room temperature for 30 min. The reaction mixture was poured into a saturated aqueous ammonium chloride solution and extracted with ethyl acetate. The extract was dried over anhydrous sodium sulfate, and the solvent was evaporated in vacuo. The residue was subjected to silica gel column chromatography, and the fraction eluted with ethyl acetate was concentrated in vacuo to give the desired product (320 mg) as an amorphous solid.

MS (ESI+, m/e) 559 (M+1)

### Reference Example 577

### tert-Butyl (3R)-3-benzyl-4-({1-(4-hydroxy-4-(methoxymethyl)tetrahydro-2H-pyran-3-yl]-5-phenyl-1H-imidazol-4-yl}carbonyl)piperazine-1-carboxylate

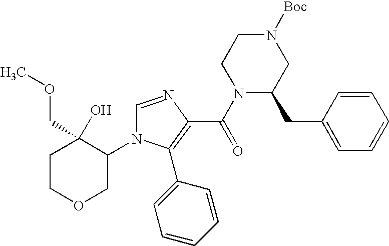

tert-Butyl (3R)-3-benzyl-4-{[1-(1,6-dioxaspiro[2.5]-4-octyl)-5-phenyl-1H-imidazol-4-yl]carbonyl}piperazine-1-carboxylate (150 mg) was dissolved in methanol (3.5 ml), sodium methoxide (28% methanol solution, 1.2 ml) was added and the mixture was stirred at 60° C. for 12 hr. The reaction mixture was poured into a 10% aqueous citric acid solution, methanol was evaporated in vacuo, and the mixture was extracted with ethyl acetate-THF. The extract was dried over anhydrous sodium sulfate, and the solvent was evaporated in vacuo. The residue was subjected to silica gel column chromatography, and the fraction eluted with ethyl acetate-methanol (4:1) was concentrated in vacuo to give the desired product (160 mg) as an amorphous solid.

MS (ESI+, m/e) 591 (M+1)

### Reference Example 578

### tert-Butyl (3R)-3-benzyl-4-[(5-phenyl-1-piperidin-3-yl-1H-imidazol-4-yl)carbonyl]piperazine-1-carboxylate

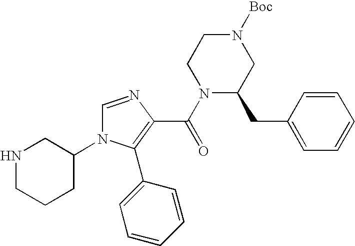

tert-Butyl (3R)-3-benzyl-4-[(1-{1-[(benzyloxy)carbonyl]piperidin-3-yl}-5-phenyl-1H-imidazol-4-yl)carbonyl]piperazine-1-carboxylate (4.30 g) was dissolved in methanol (50 ml), 10% palladium on carbon (containing 50% water) (500 mg) was added, and a catalytic hydrogenation was performed at room temperature and atmospheric pressure for 12 hr. The catalyst was filtered off, and the filtrate was concentrated in vacuo. The residue was subjected to silica gel column chromatography, and the fraction eluted with ethyl acetate-methanol (9:1) was concentrated in vacuo to give the desired product (3.33 g) as an amorphous solid.

MS (ESI+, m/e) 530 (M+1)

### Reference Example 579

### tert-Butyl (3R)-3-benzyl-4-[(5-phenyl-1-pyrrolidin-3-yl-1H-imidazol-4-yl)carbonyl]piperazine-1-carboxylate

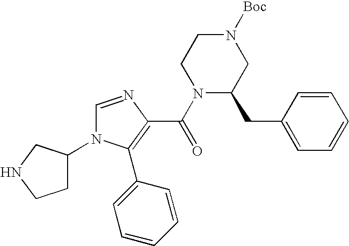

tert-Butyl (3R)-3-benzyl-4-([1-(1-benzylpyrrolidin-3-yl)-5-phenyl-1H-imidazol-4-yl]carbonyl}piperazine-1-carboxylate (3.7 g) was dissolved in methanol (100 ml), 20% palladium hydroxide on carbon (containing 50% water) (1.0 g) was added, and a catalytic hydrogenation was performed at room temperature and atmospheric pressure for 12 hr. The catalyst was filtered off, and the filtrate was concentrated in vacuo. The residue was suspended in ethyl acetate, the mixture was washed with saturated aqueous sodium bicarbonate solution, and dried over anhydrous sodium sulfate. The solvent was evaporated in vacuo and the residue was vacuum dried to give the desired product (2.1 g) as an amorphous solid.

MS (ESI+, m/e) 516 (M+1)

In the same manner as in Reference Example 579, the following compounds (Reference Examples 580 to 583) were obtained.

### Reference Example 580

### tert-Butyl (3R)-3-benzyl-4-({5-phenyl-1-[(3S)-pyrrolidin-3-yl]-1H-imidazol-4-yl}carbonyl)piperazine-1-carboxylate

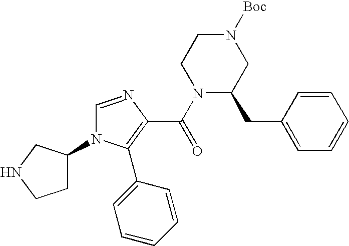

MS (ESI+, m/e) 516 (M+1)

### Reference Example 581

### tert-Butyl (3R)-3-benzyl-4-({5-phenyl-1-[(3R)-pyrrolidin-3-yl]-1H-imidazol-4-yl}carbonyl)piperazine-1-carboxylate

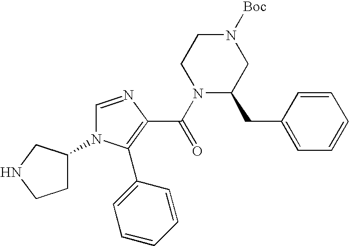

MS (ESI+, m/e) 516 (M+1)

### Reference Example 582

### tert-Butyl (3R)-3-benzyl-4-({1-[(4-hydroxypiperidin-4-yl)methyl]-5-phenyl-1H-imidazol-4-yl)carbonyl)piperazine-1-carboxylate

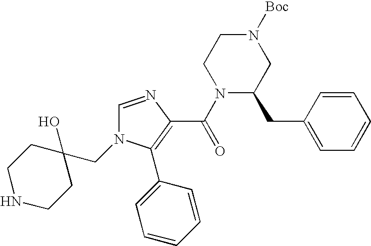

MS (ESI+, m/e) 560 (M+1)

### Reference Example 583

### tert-Butyl (3R)-3-benzyl-4-({1-[(3-hydroxypiperidin-3-yl)methyl]-5-phenyl-1H-imidazol-4-yl}carbonyl)piperazine-1-carboxylate

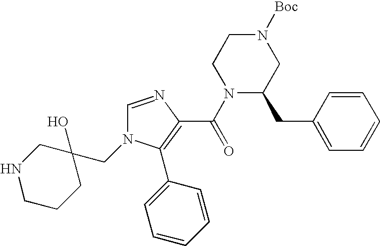

MS (ESI+, m/e) 560 (M+1)

### Reference Example 584

### (2R)-2,4-Dibenzyl-1-{[5-phenyl-1-(piperidin-2-ylmethyl)-1H-imidazol-4-yl]carbonyl}piperazine

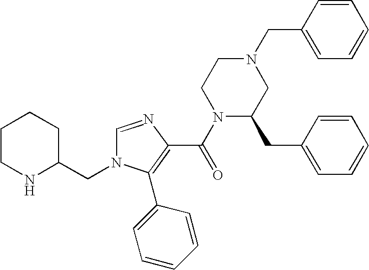

A mixture of tert-butyl 2-[(4-{[(2R)-2,4-dibenzylpiperazin-1-yl]carbonyl}-5-phenyl-1H-imidazol-1-yl)methyl]piperidine-1-carboxylate (1.58 g), TFA (9.0 ml) and dichloromethane (9.0 ml) was stirred at room temperature for 2 days, and concentrated in vacuo. The residue was dissolved in ethyl acetate, and the mixture was washed successively with an aqueous potassium carbonate solution and brine, and dried over anhydrous sodium sulfate. The solvent was evaporated in vacuo to give the desired product (1.12 g).

MS (ESI+, m/e) 534 (M+1)

### Reference Example 585

### tert-Butyl ((3R)-3-benzyl-4-({5-phenyl-1-[1-(phenylsulfonyl)piperidin-3-yl]-1H-imidazol-4-yl}carbonyl)piperazine-1-carboxylate

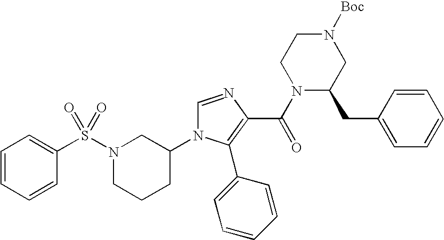

tert-Butyl (3R)-3-benzyl-4-[(5-phenyl-1-piperidin-3-yl-1H-imidazol-4-yl)carbonyl]piperazine-1-carboxylate (212 mg) and triethylamine (61 mg) were dissolved in THF (10 ml) and the mixture was ice-cooled. Benzenesulfonyl chloride (85 mg) was added and the mixture was stirred at room temperature for 15 hr. The reaction mixture was poured into water, and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-methanol (1:0 to 19:1) was concentrated in vacuo to give the desired product (138 mg) as an amorphous solid.

MS (ESI+, m/e) 670 (M+1)

### Reference Example 586

### tert-Butyl (3R)-3-benzyl-4-[(1-{1-[(6-methoxypyridin-3-yl)sulfonyl]piperidin-3-yl}-5-phenyl-1H-imidazol-4-yl)carbonyl]piperazine-1-carboxylate

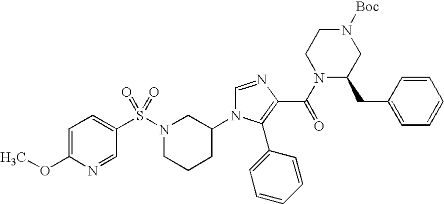

tert-Butyl (3R)-3-benzyl-4-[(5-phenyl-1-piperidin-3-yl-1H-imidazol-4-yl)carbonyl]piperazine-1-carboxylate (128 mg) and triethylamine (0.05 ml) were dissolved in THF (3 ml), a solution of 6-methoxypyridine-3-sulfonyl chloride (59 mg) in THF (2 ml) was added and the mixture was stirred at room temperature for 1 hr. The reaction mixture was poured into aqueous sodium bicarbonate solution and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate was concentrated in vacuo to give the desired product (165 mg) as an amorphous solid.

MS (ESI+, m/e) 701 (M+1)

In the same manner as in Reference Example 586, the following compounds (Reference Examples 587 to 604) were obtained.

### Reference Example 587

### tert-Butyl (3R)-4-({1-[1-(cyclopropylsulfonyl)piperidin-3-yl]-5-phenyl-1H-imidazol-4-yl}carbonyl)-3-benzylpiperazine-1-carboxylate

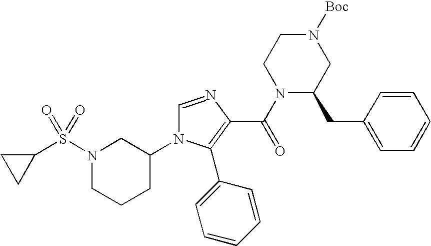

MS (ESI+, m/e) 634 (M+1)

### Reference Example 588

### tert-Butyl (3R)-3-benzyl-4-({5-phenyl-1-[1-(pyridin-2-ylsulfonyl)piperidin-3-yl)-1H-imidazol-4-yl}carbonyl)piperazine-1-carboxylate

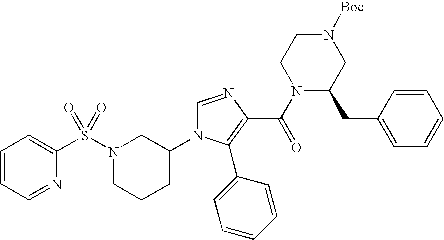

MS (ESI+, m/e) 671 (M+1)

### Reference Example 589

### tert-Butyl (3R)-3-benzyl-4-[(1-{1-[(1-methyl-1H-pyrazol-4-yl)sulfonyl]piperidin-3-yl)-5-phenyl-1H-imidazol-4-yl)carbonyl]piperazine-1-carboxylate

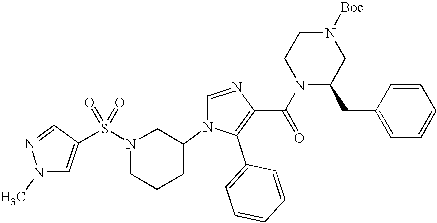

MS (ESI+, m/e) 674 (M+1)

### Reference Example 590

### tert-Butyl (3R)-3-benzyl-4-({5-phenyl-1-[1-(2-thienylsulfonyl)piperidin-3-yl]-1H-imidazol-4-yl}carbonyl)piperazine-1-carboxylate

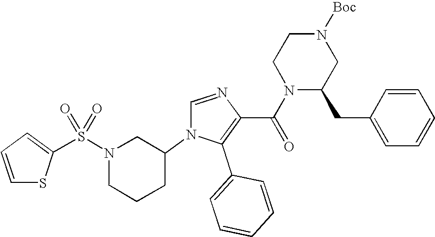

MS (ESI+, m/e) 676 (M+1)

### Reference Example 591

### tert-Butyl (3R)-3-benzyl-4-[(5-phenyl-1-{1-[(2,2,2-trifluoroethyl)sulfonyl]piperidin-3-yl}-1H-imidazol-4-yl)carbonyl]piperazine-1-carboxylate

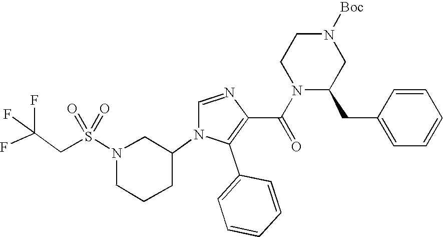

MS (ESI+, m/e) 676 (M+1)

### Reference Example 592

### tert-Butyl (3R)-3-benzyl-4-({1-[1-(ethylsulfonyl)piperidin-3-yl]-5-phenyl-1H-imidazol-4-yl}carbonyl)piperazine-1-carboxylate

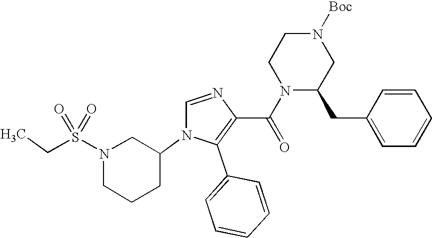

MS (ESI+, m/e) 622 (M+1)

### Reference Example 593

### tert-Butyl (3R)-3-benzyl-4-[(1-{1-[(2,4-dimethoxyphenyl)sulfonyl]piperidin-3-yl}-5-phenyl-1H-imidazol-4-yl)carbonyl]piperazine-1-carboxylate

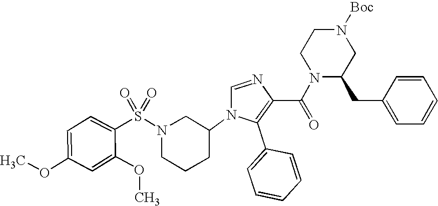

MS (ESI+, m/e) 730 (M+1)

### Reference Example 594

### tert-Butyl (3R)-3-benzyl-4-({5-phenyl-1-[1-(pyridin-3-ylsulfonyl)piperidin-3-yl]-1H-imidazol-4-yl)carbonyl)piperazine-1-carboxylate

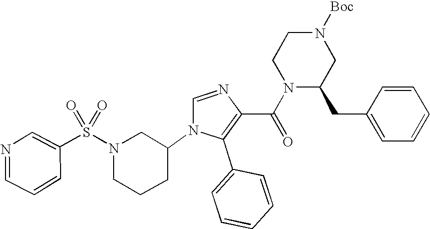

MS (ESI+, m/e) 671 (M+1)

### Reference Example 595

### tert-Butyl (3R)-3-benzyl-4-[(1-{1-[(1,2-dimethyl-1H-imidazol-4-yl)sulfonyl]piperidin-3-yl}-5-phenyl-1H-imidazol-4-yl)carbonyl]piperazine-1-carboxylate

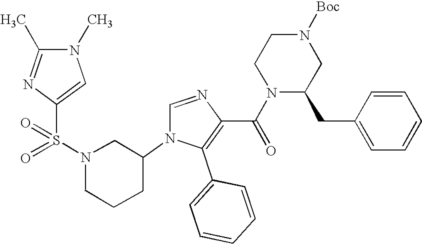

MS (ESI+, m/e) 688 (M+1)

### Reference Example 596

### tert-Butyl (3R)-3-benzyl-4-{[1-(1-{[5-(methoxycarbonyl)-2-furyl]sulfonyl}piperidin-3-yl)-5-phenyl-1H-imidazol-4-yl]carbonyl}piperazine-1-carboxylate

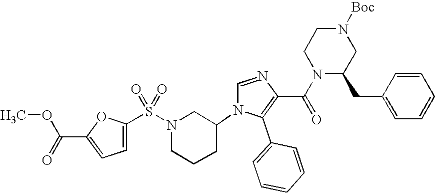

MS (ESI+, m/e) 718 (M+1)

### Reference Example 597

### tert-Butyl -(3R)-3-benzyl-4-[(1-{1-[(5-chloro-2-thienyl)sulfonyl]piperidin-3-yl}-5-phenyl-1H-imidazol-4-yl)carbonyl]piperazine-1-carboxylate

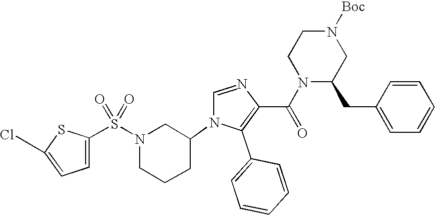

MS (ESI+, m/e) 711 (M+1)

### Reference Example 598

### tert-Butyl (3R)-3-benzyl-4-({5-phenyl-1-[1-(phenylsulfonyl)pyrrolidin-3-yl]-1H-imidazol-4-yl}carbonyl)piperazine-1-carboxylate

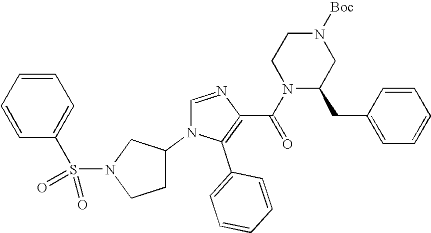

MS (ESI+, m/e) 656 (M+1)

### Reference Example 599

### tert-Butyl (3R)-3-benzyl-4-({1-[(3S)-1-(methylsulfonyl)pyrrolidin-3-yl]-5-phenyl-1H-imidazol-4-yl}carbonyl)piperazine-1-carboxylate

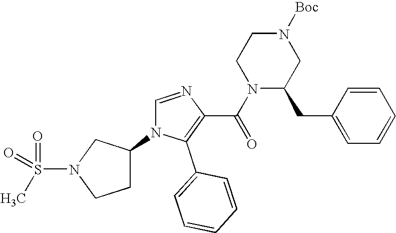

MS (ESI+, m/e) 594 (M+1)

### Reference Example 600

### tert-Butyl (3R)-3-benzyl-4-({1-[(3R)-1-(methylsulfonyl)pyrrolidin-3-yl]-5-phenyl-1H-imidazol-4-yl)carbonyl)piperazine-1-carboxylate

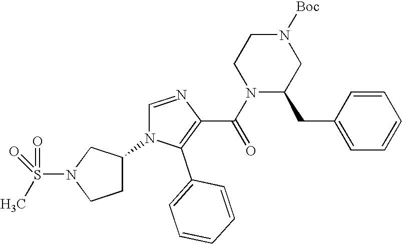

MS (ESI+, m/e) 594 (M+1)

### Reference Example 601

### tert-Butyl (3R)-3-benzyl-4-[(1-{[4-hydroxy-1-(methylsulfonyl)piperidin-4-yl]methyl}-5-phenyl-1H-imidazol-4-yl)carbonyl]piperazine-1-carboxylate

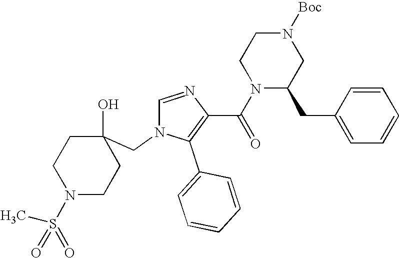

MS (ESI+, m/e) 638 (M+1)

### Reference Example 602

### tert-Butyl (3R)-3-benzyl-4-[(1-{[4-hydroxy-1-(phenylsulfonyl)piperidin-4-yl]methyl}-5-phenyl-1H-imidazol-4-yl)carbonyl]piperazine-1-carboxylate

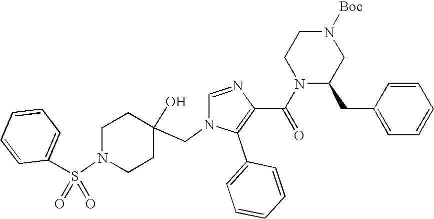

MS (ESI+, m/e) 700 (M+1)

### Reference Example 603

### tert-Butyl (3R)-3-benzyl-4-[(1-{[3-hydroxy-1-(methylsulfonyl)piperidin-3-yl]methyl}-5-phenyl-1H-imidazol-4-yl)carbonyl]piperazine-1-carboxylate

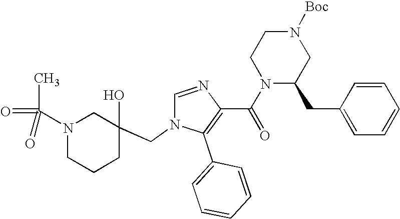

MS (ESI+, m/e) 638 (M+1)

### Reference Example 604

### tert-Butyl (3R)-3-benzyl-4-[(1-{[3-hydroxy-1-(phenylsulfonyl)piperidin-3-yl]methyl)-5-phenyl-1H-imidazol-4-yl)carbonyl]piperazine-1-carboxylate

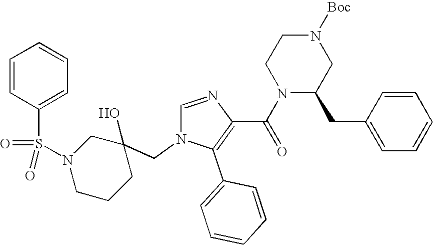

MS (ESI+, m/e) 700 (M+1)

### Reference Example 605

### tert-Butyl (3R)-3-benzyl-4-({1-[(3S)-1-(2-hydroxy-2-methylpropanoyl)pyrrolidin-3-yl]-5-phenyl-1H-imidazol-4-yl}carbonyl)piperazine-1-carboxylate

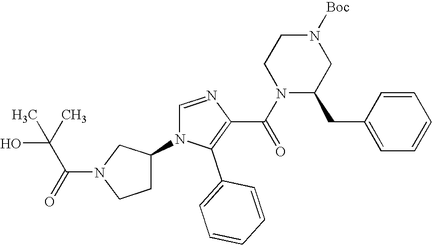

A solution of tert-butyl (3R)-3-benzyl-4-({5-phenyl-1-[(3S)-pyrrolidin-3-yl]-1H-imidazol-4-yl}carbonyl)piperazine-1-carboxylate (150 mg), 2-hydroxy-2-methylpropionic acid (36 mg), WSC.HCl (61 mg), HOBt (48 mg) and DMF (5 ml) was stirred at room temperature for 15 hr. The reaction mixture was poured into water, and extracted with ethyl acetate. The extract was washed successively with a 10% aqueous citric acid solution, saturated aqueous sodium bicarbonate solution and brine, and dried over anhydrous sodium sulfate, and the solvent was evaporated in vacuo. The residue was subjected to basic silica gel column chromatography, and the fraction eluted with ethyl acetate-methanol (1:0 to 9:1) was concentrated in vacuo to give the desired product (56 mg) as an amorphous solid.

MS (ESI+, m/e) 602 (M+1)

In the same manner as in Reference Example 605, the following compound (Reference Example 606) was obtained.

### Reference Example 606

### tert-Butyl (3R)-3-benzyl-4-({1-[(3R)-1-(2-hydroxy-2-methylpropanoyl)pyrrolidin-3-yl]-5-phenyl-1H-imidazol-4-yl}carbonyl)piperazine-1-carboxylate

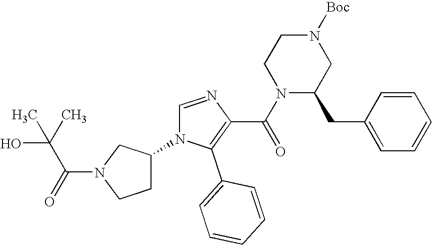

MS (ESI+, m/e) 602 (M+1)

### Reference Example 607

### tert-Butyl (3R)-4-({1-[(3R)-1-acetylpyrrolidin-3-yl]-5-phenyl-1H-imidazol-4-yl}carbonyl)-3-benzylpiperazine-1-carboxylate

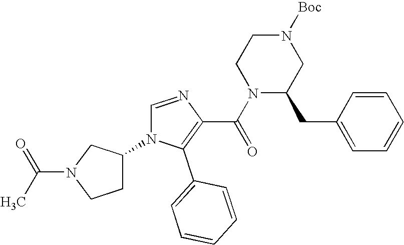

tert-Butyl (3R)-3-benzyl-4-({5-phenyl-1-[(3R)-pyrrolidin-3-yl]-1H-imidazol-4-yl}carbonyl)piperazine-1-carboxylate (140 mg) and triethylamine (41 mg) were dissolved in THF (5 ml) and the mixture was ice-cooled. Acetic anhydride (33 mg) was added and the mixture was stirred at room temperature for 15 hr. The reaction mixture was poured into water, and the mixture was extracted with ethyl acetate. The extract was washed successively with a 10% aqueous citric acid-solution, saturated aqueous sodium bicarbonate solution and brine, and dried over anhydrous sodium sulfate, and the solvent was evaporated in vacuo. The residue was subjected to silica gel column chromatography, and the fraction eluted with ethyl acetate-methanol (1:0 to 9:1) was concentrated in vacuo to give the desired product (47 mg) as an amorphous solid.

MS (ESI+, m/e) 558 (M+1)

### Reference Example 608

### tert-Butyl (3R)-4-{[1-(1-benzoylpyrrolidin-3-yl)-5-phenyl-1H-imidazol-4-yl]carbonyl}-3-benzylpiperazine-1-carboxylate

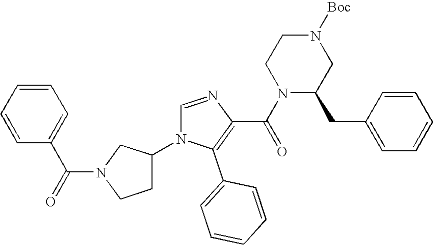

tent-Butyl (3R)-3-benzyl-4-[(5-phenyl-1-pyrrolidin-3-yl-1H-imidazol-4-yl)carbonyl]piperazine-1-carboxylate (140 mg) was dissolved in DMA (2 ml), benzoyl chloride (59 mg) was added and the mixture was stirred at room temperature for 1 hr. The reaction mixture was poured into aqueous sodium bicarbonate solution and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-methanol (1:1) was concentrated in vacuo to give the desired product (110 mg) as an amorphous solid.

MS (ESI+, m/e) 620 (M+1)

### Reference Example 609

### tert-Butyl (3R)-4-({1-[(1-benzoyl-4-hydroxypiperidin-4-yl)methyl]-5-phenyl-1H-imidazol-4-yl}carbonyl)-3-benzylpiperazine-1-carboxylate

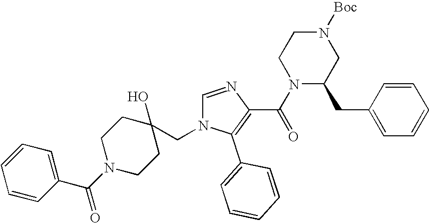

tert-Butyl (3R)-3-benzyl-4-({1-[(1-benzyl-4-hydroxypiperidin-4-yl)methyl]-5-phenyl-1H-imidazol-4-yl}carbonyl)piperazine-1-carboxylate (125 mg) and triethylamine (2 ml) were dissolved in THF (3.5 ml), benzoyl chloride (38 mg) was added and the mixture was stirred at room temperature for 2 hr. The reaction mixture was poured into aqueous sodium bicarbonate solution and extracted with ethyl acetate. The extract was washed with brine, and dried over anhydrous sodium sulfate, and the solvent was evaporated in vacuo. The residue was subjected to silica gel column chromatography, and the fraction eluted with ethyl acetate-hexane (1:1) was concentrated in vacuo to give the desired product (110 mg) as an amorphous solid.

MS (ESI+, m/e) 664 (M+1)

In the same manner as in Reference Example 609, the following compounds (Reference Examples 610 and 611) were obtained.

### Reference Example 610

### tert-Butyl (3R)-4-({1-[(1-benzoyl-3-hydroxypiperidin-3-yl)methyl]-5-phenyl-1H-imidazol-4-yl}carbonyl)-3-benzylpiperazine-1-carboxylate

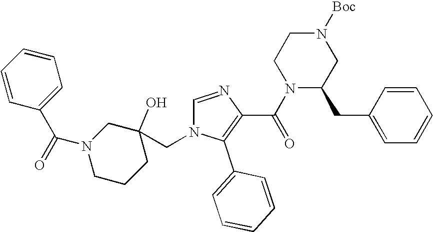

MS (ESI+, m/e) 664 (M+1)

### Reference Example 611

### tert-Butyl (3R)-4-{[1-(1-benzoylpiperidin-3-yl)-5-phenyl-1H-imidazol-4-yl]carbonyl}-3-benzylpiperazine-1-carboxylate

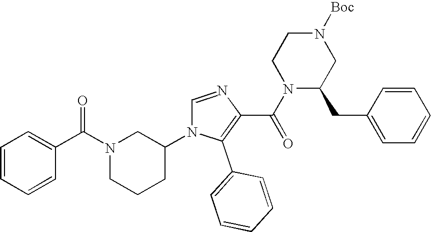

MS (ESI+, m/e) 634 (M+1)

### Reference Example 612

### tert-Butyl (3R)-3-benzyl-4-({1-[1-(cyclopropylmethyl)piperidin-3-yl]-5-phenyl-1H-imidazol-4-yl}carbonyl)piperazine-1-carboxylate

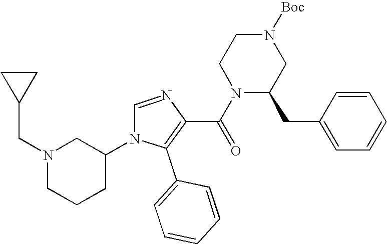

To a mixture of tert-butyl (3R)-3-benzyl-4-[(5-phenyl-1-piperidin-3-yl-1H-imidazol-4-yl)carbonyl]piperazine-1-carboxylate (212 mg), potassium carbonate (110 mg) and DMF (5 ml) was added (bromomethyl)cyclopropane (40 μl) and the mixture was stirred at room temperature for 12 hr. The reaction mixture was concentrated in vacuo, and the residue was partitioned between ethyl acetate and water. The organic layer was washed with brine, and dried over anhydrous sodium sulfate, and the solvent was evaporated in vacuo. The residue was subjected to basic silica gel column chromatography, and the fraction eluted with ethyl acetate was concentrated in vacuo to give the desired product (187 mg) as an amorphous solid.

MS (ESI+, m/e) 584 (M+1)

In the same manner as in Reference Example 612, the following compounds (Reference Examples 613 and 614) were obtained.

### Reference Example 613

### tert-Butyl (3R)-3-benzyl-4-{[1-(1-methylpiperidin-3-yl)-5-phenyl-1H-imidazol-4-yl]carbonyl}piperazine-1-carboxylate

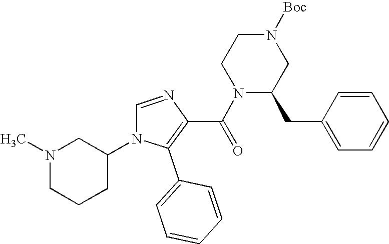

MS (ESI+, m/e) 544 (M+1)

### Reference Example 614

### tert-Butyl (3R)-3-benzyl-4-({1-[1-(3-methoxypropyl)piperidin-3-yl]-5-phenyl-1H-imidazol-4-yl}carbonyl)piperazine-1-carboxylate

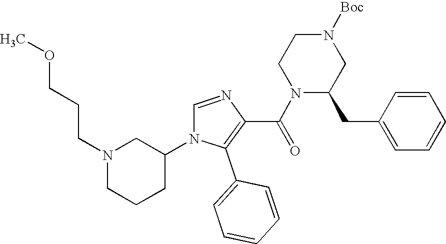

MS (ESI+, m/e) 602 (M+1)

### Reference Example 615

### (2R)-2,4-Dibenzyl-1-[(1-{[1-(2-methoxyethyl)piperidin-2-yl]methyl}-5-phenyl-1H-imidazol-4-yl)carbonyl]piperazine

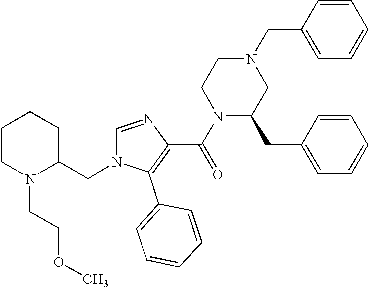

A mixture of (2R)-2,4-dibenzyl-1-{[5-phenyl-1-(piperidin-2-ylmethyl)-1H-imidazol-4-yl]carbonyl}piperazine (315 mg), 1-bromo-2-methoxyethane (83 μl), potassium iodide (50 mg), potassium carbonate (200 mg) and DMF (7.0 ml) was stirred at 70° C. for 13 hr, and diluted with ethyl acetate. The reaction mixture was washed successively with aqueous potassium carbonate solution and brine, and dried over anhydrous sodium sulfate. The solvent was evaporated in vacuo. The residue was subjected to basic silica gel column chromatography, and the fraction eluted with ethyl acetate-hexane (3:7 to 1:0) was concentrated in vacuo to give the desired product (349 mg).

MS (ESI+, m/e) 592 (M+1)

### Reference Example 616

### (2R)-2,4-Dibenzyl-1-{[1-({1-[3-(5-methyl-2-furyl)butyl]piperidin-2-yl}methyl)-5-phenyl-1H-imidazol-4-yl]carbonyl}piperazine

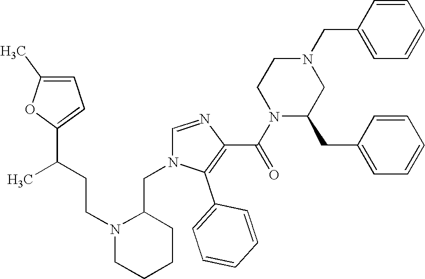

A mixture of (2R)-2,4-dibenzyl-1-{[5-phenyl-1-(piperidin-2-ylmethyl)-1H-imidazol-4-yl]carbonyl}piperazine (210 mg), 3-(5-methyl-2-furyl)butylaldehyde (120 mg), acetic acid (1.0 ml) and 1,2-dichloroethane (3.0 ml) was stirred at room temperature for 1 hr, sodium triacetoxyborohydride (370 mg) was added and the mixture was stirred at room temperature for additional 13 hr. The reaction mixture was poured into a 1 N aqueous sodium hydroxide solution, and the mixture was extracted with ethyl acetate. The extract was washed with brine, and dried over anhydrous magnesium sulfate. The solvent was evaporated in vacuo. The residue was subjected to basic silica gel column chromatography, and the fraction eluted with ethyl acetate-hexane (1:9 to 1:0) was concentrated in vacuo to give the desired product (125 mg).

MS (ESI+, m/e) 670 (M+1)

### Reference Example 617

### tert-Butyl (3R)-3-benzyl-4-{[5-phenyl-1-(1-phenylpiperidin-3-yl)-1H-imidazol-4-yl]carbonyl}piperazine-1-carboxylate

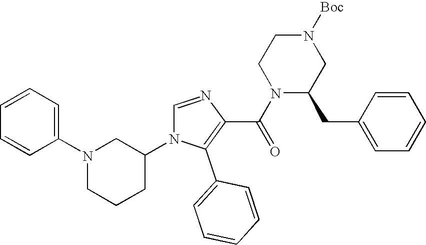

tert-Butyl (3R)-3-benzyl-4-[(5-phenyl-1-piperidin-3-yl-1H-imidazol-4-yl)carbonyl]piperazine-1-carboxylate (125 mg), BINAP (28 mg), sodium tert-butoxide (43 mg), Pd₂(dba)₃ (14 mg) and bromobenzene (50 mg) were mixed with toluene (3 ml) under argon atmosphere. After stirring at 80° C. for 5 hr, and the mixture was diluted with ethyl acetate, washed with water, and dried over anhydrous sodium sulfate, and the solvent was evaporated in vacuo. The residue was subjected to silica gel column chromatography, and the fraction eluted with ethyl acetate was concentrated in vacuo to give the desired product (110 mg) as an amorphous solid.

MS (ESI+, m/e) 606 (M+1)

In the same manner as in Reference Example 617, the following compounds (Reference Examples 618 and 619) were obtained.

### Reference Example 618

### tert-Butyl (3R)-3-benzyl-4-([5-phenyl-1-(1-(pyridin-2-yl)piperidin-3-yl)-1H-imidazol-4-yl]carbonyl)piperazine-1-carboxylate

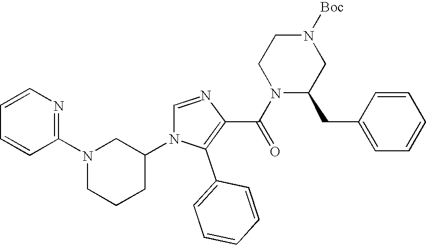

MS (ESI+, m/e) 607 (M+1)

### Reference Example 619

### tert-Butyl (3R)-3-benzyl-4-[(1-{1-[4-(methylsulfonyl)phenyl]piperidin-3-yl}-5-phenyl-1H-imidazol-4-yl)carbonyl]piperazine-1-carboxylate

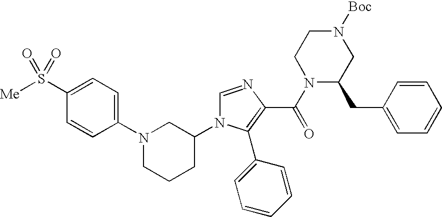

MS (ESI+, m/e) 584 (M+1)

### Reference Example 620

### tert-Butyl (3R)-3-benzyl-4-{[1-(1-methyl-2-oxoazepan-3-yl)-5-phenyl-1H-imidazol-4-yl]carbonyl}piperazine-1-carboxylate

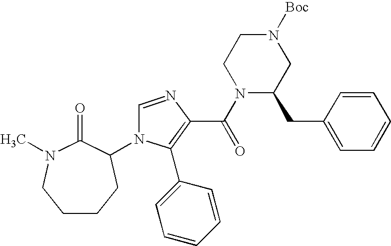

tert-Butyl (3R)-3-benzyl-4-([1-(2-oxoazepan-3-yl)-5-phenyl-1H-imidazol-4-yl]carbonyl)piperazine-1-carboxylate (167 mg) was dissolved in DMF (5 ml), sodium hydride (60% in oil) (24 mg) was added, and the mixture was stirred at room temperature for 1 hr. Methyl iodide (20 μl) was added to the reaction mixture and the mixture was stirred at 60° C. for additional 3 hr. Water (1 drop) was added to the reaction mixture and the mixture was concentrated in vacuo. The residue was partitioned between ethyl acetate and water. The organic layer was washed with brine, and dried over anhydrous sodium sulfate, and the solvent was evaporated in vacuo. The residue was subjected to silica gel column chromatography, and the fraction eluted with ethyl acetate-methanol (9:1) was concentrated in vacuo to give the desired product (147 mg) as an amorphous solid.

MS (ESI+, m/e) 572 (M+1)

In the same manner as in Reference Example 620, the following compounds (Reference Examples 621 and 622) were obtained.

### Reference Example 621

### tert-Butyl (3R)-3-benzyl-4-({1-[1-(cyclopropylmethyl)-2-oxoazepan-3-yl]-5-phenyl-1H-imidazol-4-yl}carbonyl)piperazine-1-carboxylate

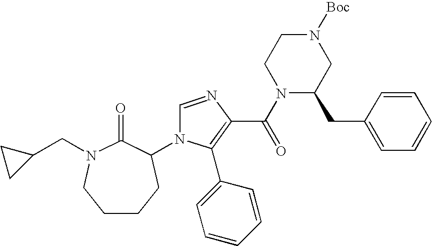

MS (ESI+, m/e) 612 (M+1)

### Reference Example 622

### tert-Butyl (3R)-3-benzyl-4-[(1-{1-[4-(methylsulfonyl)benzyl]-2-oxoazepan-3-yl}-5-phenyl-1H-imidazol-4-yl)carbonyl]piperazine-1-carboxylate

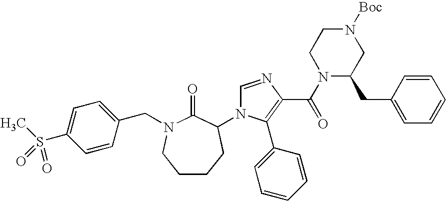

MS (ESI+, m/e) 726 (M+1)

### Reference Example 623

### tert-Butyl (3R)-3-benzyl-4-{1-[1-(1-{[5-(ethoxycarbonyl)-2-furyl]methyl}-2-oxoazepan-3-yl)-5-phenyl-1H-imidazol-4-yl]vinyl)piperazine-1-carboxylate and 5-{[3-(4-{1-[(2R)-2-benzyl-4-(tert-butoxycarbonyl)piperazin-1-yl]vinyl}-5-phenyl-1H-imidazol-1-yl)-2-oxoazepan-1-yl]methyl}furan-2-carboxylic acid

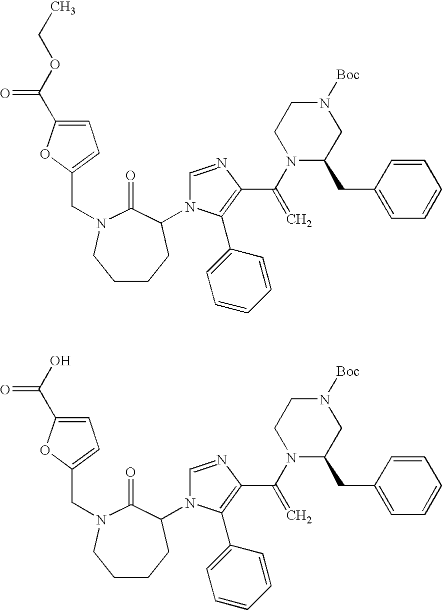

tert-Butyl (3R)-3-benzyl-4-{[1-(2-oxoazepan-3-yl)-5-phenyl-1H-imidazol-4-yl]carbonyl}piperazine-1-carboxylate (167 mg) was dissolved in DMF (5 ml), sodium hydride (60% in oil) (24 mg) was added and the mixture was stirred at room temperature for 1 hr. Ethyl 5-(chloromethyl)furan-2-carboxylate (0.05 ml) was added to the reaction mixture and the mixture was stirred at 60° C. for additional 3 hr. Water (1 drop) was added to the reaction mixture and the mixture was concentrated in vacuo. The residue was partitioned between ethyl acetate and water. The organic layer was washed with brine, and dried over anhydrous sodium sulfate, and the solvent was evaporated in vacuo to give desired mixture (223 mg) as an amorphous solid.

MS (ESI+, m/e) 710 (M+1), 682 (M+1)

### Reference Example 624

### tert-Butyl (3R)-3-benzyl-4-{[1-(2-oxo-1-phenylazepan-3-yl)-5-phenyl-1H-imidazol-4-yl]carbonyl}piperazine-1-carboxylate

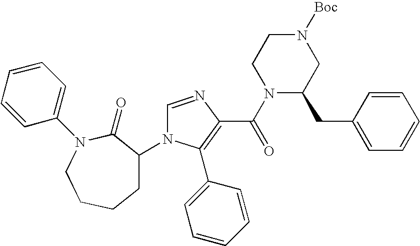

A mixture of tert-butyl (3R)-3-benzyl-4-{[1-(2-oxoazepan-3-yl)-5-phenyl-1H-imidazol-4-yl]carbonyl}piperazine-1-carboxylate (100 mg), iodobenzene (73 mg), copper iodide (34 mg), ethylenediamine (12 μl), potassium phosphate (152 mg) and dioxane (3 ml) was stirred at 100° C. for 16 hr. The insoluble material was filtered off on Celite and the filtrate was concentrated in vacuo. The residue was subjected to silica gel column chromatography, and the fraction eluted with ethyl acetate-methanol (93:7 to 4:1) was concentrated in vacuo to give the desired product (13.5 mg).

MS (ESI+, m/e) 634 (M+1)

In the same manner as in Reference Example 624, the following compound (Reference Example 625) was obtained.

### Reference Example 625

### tert-Butyl (3R)-3-benzyl-4-({1-[2-oxo-1-(2-thienyl)azepan-3-yl]-5-phenyl-1H-imidazol-4-yl}carbonyl)piperazine-1-carboxylate

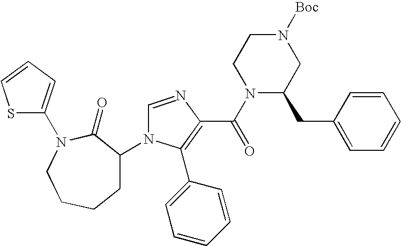

MS (ESI+, m/e) 640 (M+1)

### Reference Example 626

### tert-Butyl (3R)-3-benzyl-4-{[1-(2-hydroxyethyl)-3,4-diphenyl-1H-pyrazol-5-yl]carbonyl}piperazine-1-carboxylate and tert-butyl (3R)-3-benzyl-4-{[1-(2-hydroxyethyl)-4,5-diphenyl-1H-pyrazol-3-yl]carbonyl}piperazine-1-carboxylate

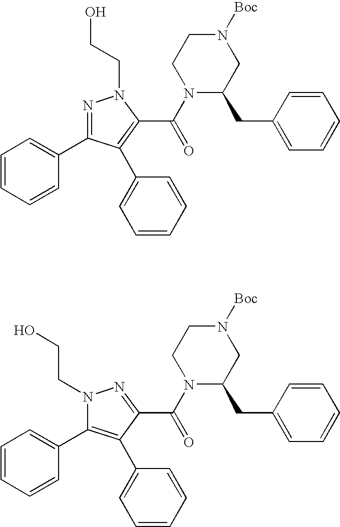

To a solution of tert-butyl (3R)-3-benzyl-4-[(3,4-diphenyl-1H-pyrazol-5-yl)carbonyl]piperazine-1-carboxylate (523 mg), 2-bromoethanol (0.085 ml) sand DMA (15 ml) was added cesium carbonate (652 mg). After stirring at 65° C. for 3 hr, the reaction mixture was poured into water, and the mixture was extracted with ethyl acetate. The extract was washed with brine, and dried over anhydrous sodium sulfate, and the solvent was evaporated in vacuo. The residue was subjected to silica gel column chromatography, and the fraction eluted with acetic acid was concentrated in vacuo to give tert-butyl (3R)-3-benzyl-4-{[1-(2-hydroxyethyl)-3,4-diphenyl-1H-pyrazol-5-yl]carbonyl}piperazine-1-carboxylate (340 mg) as an amorphous solid, and tert-butyl (3R)-3-benzyl-4-{[1-(2-hydroxyethyl)-4,5-diphenyl-1H-pyrazol-3-yl]carbonyl}piperazine-1-carboxylate (144 mg) as crystals.

MS (ESI+, m/e) 567 (M+1)

MS (ESI+, m/e) 567 (M+1)

In the same manner as in Reference Example 626, the following compound (Reference Example 627) was obtained.

### Reference Example 627

### tert-Butyl (3R)-3-benzyl-4-{[1-(2-ethoxy-2-oxoethyl)-4,5-diphenyl-1H-pyrazol-3-yl]carbonyl}piperazine-1-carboxylate and tert-butyl (3R)-3-benzyl-4-{[1-(2-ethoxy-2-oxoethyl)-3,4-diphenyl-1H-pyrazol-5-yl]carbonyl}piperazine-1-carboxylate

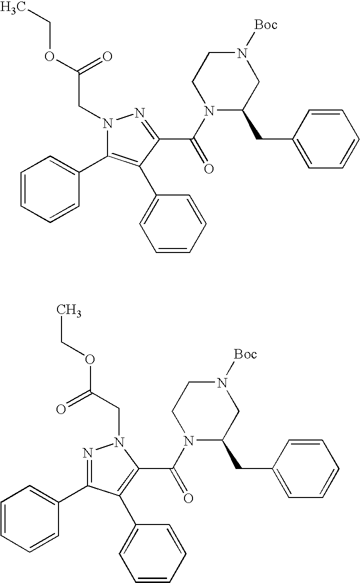

MS (ESI+, m/e) 609 (M+1)

MS (ESI+, m/e) 609 (M+1)

### Reference Example 628

### (3-{[(2R)-2-benzyl-4-(tert-butoxycarbonyl)piperazin-1-yl]carbonyl}-4,5-diphenyl-1H-pyrazol-1-yl)acetic acid

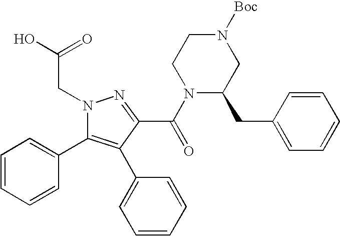

tert-Butyl (3R)-3-benzyl-4-{[1-(2-ethoxy-2-oxoethyl)-4,5-diphenyl-1H-pyrazol-3-yl]carbonyl}piperazine-1-carboxylate (526 mg) was suspended in ethanol (15 ml), and a 4 N aqueous sodium hydroxide solution (1.1 ml) was added. After heating under reflux for 1 hr, the mixture was weakly acidified (pH 3) with 1 N hydrochloric acid, and extracted with ethyl acetate. The extract was washed with brine, and dried over anhydrous sodium sulfate, and the solvent was evaporated in vacuo to give the desired product (495 mg) as an amorphous solid.

MS (ESI+, m/e) 581 (M+1)

In the same manner as in Reference Example 628, the following compound (Reference Example 629) was obtained.

### Reference Example 629

### (5-{[(2R)-2-benzyl-4-(tert-butoxycarbonyl)piperazin-1-yl]carbonyl)-3,4-diphenyl-1H-pyrazol-1-yl)acetic acid

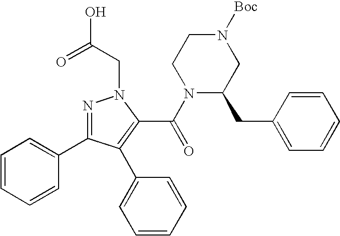

MS (ESI+, m/e) 581 (M+1)

### Reference Example 630

### tert-Butyl (3R)-3-benzyl-4-[(1-{2-[(2-hydroxyethyl)amino]-2-oxoethyl)-4,5-diphenyl-1H-pyrazol-3-yl)carbonyl]piperazine-1-carboxylate

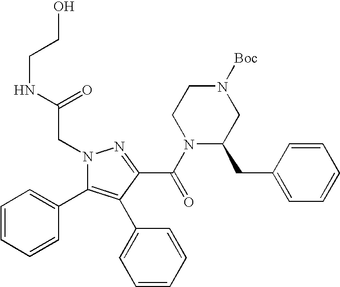

A solution of (3-{[(2R)-2-benzyl-4-(tert-butoxycarbonyl)piperazin-1-yl]carbonyl)-4,5-diphenyl-1H-pyrazol-1-yl)acetic acid (240 mg), 2-aminoethanol (0.027 ml), WSC.HCl (94 mg), HOBt (66 mg) and DMF (5 ml) was stirred at room temperature for 15 hr, 10% aqueous sodium bicarbonate solution (50 ml) was added, and the mixture was extracted with ethyl acetate. The extract was washed with brine, dried over anhydrous magnesium sulfate, and concentrated in vacuo. The residue was subjected to silica gel column chromatography, and the fraction eluted with ethyl acetate-methanol (9:1) was concentrated in vacuo to give the desired product (250 mg) as an amorphous solid.

MS (ESI+, m/e) 624 (M+1)

In the same manner as in Reference Example 630, the following compounds (Reference Examples 631 and 632) were obtained.

### Reference Example 631

### tert-Butyl (3R)-3-benzyl-4-[(1-{2-[(2-hydroxyethyl)amino]-2-oxoethyl)-3,4-diphenyl-1H-pyrazol-5-yl)carbonyl]piperazine-1-carboxylate

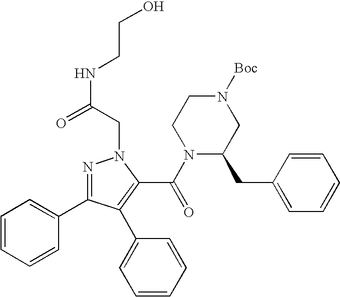

MS (ESI+, m/e) 624 (M+1)

### Reference Example 632

### tert-Butyl (3R)-4-{[1-(2-amino-2-oxoethyl)-3,4-diphenyl-1H-pyrazol-5-yl]carbonyl}-3-benzylpiperazine-1-carboxylate

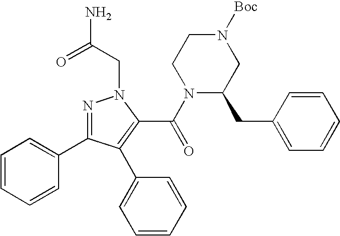

MS (ESI+, m/e) 580 (M+1)

### Reference Example 633

### tert-Butyl (3R)-3-[(isopropylamino)methyl]-4-[(5-methyl-1,2-diphenyl-1H-pyrrol-3-yl)carbonyl]piperazine-1-carboxylate

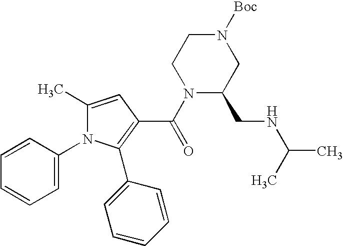

A solution of (2S)-4-benzyl-1-[(5-methyl-1,2-diphenyl-1H-pyrrol-3-yl)carbonyl]piperazine-2-carbaldehyde (1.50 g), isopropylamine (374 mg), acetic acid (380 mg), dichloromethane (12 ml) and DMF (6 ml) was stirred at room temperature for 40 min, sodium triacetoxyborohydride (1.34 g) was added and the mixture was stirred at room temperature for additional 15 hr. The reaction mixture was poured into saturated aqueous sodium bicarbonate solution, and the mixture was extracted with ethyl acetate. The extract was washed successively with water and brine, and dried over anhydrous magnesium sulfate. The solvent was evaporated in vacuo, and the crystals were collected by filtration to give the desired product (1.40 g).

MS (ESI+, m/e) 517 (M+1)

In the same manner as in Reference Example 633, the following compounds (Reference Examples 634 to 636) were obtained.

### Reference Example 634

### tert-Butyl (3R)-3-{[4-(ethoxycarbonyl)piperidin-1-yl]methyl}-4-[(5-methyl-1,2-diphenyl-1H-pyrrol-3-yl)carbonyl]piperazine-1-carboxylate

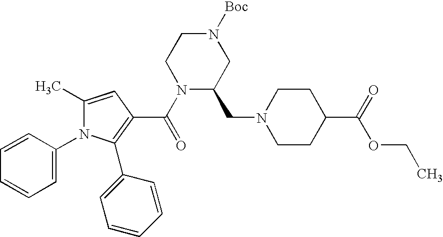

MS (ESI+, m/e) 615 (M+1)

### Reference Example 635

### tert-Butyl (3R)-3-{[(2-hydroxyethyl)amino[methyl}-4-[(5-methyl-1,2-diphenyl-1H-pyrrol-3-yl)carbonyl]piperazine-1-carboxylate

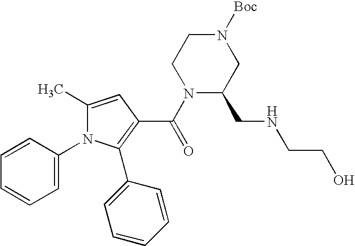

MS (ESI+, m/e) 519 (M+1)

### Reference Example 636

### tert-Butyl (3R)-4-{[1-(2,3-dihydro-1H-inden-2-yl)-5-phenyl-1H-imidazol-4-yl]carbonyl}-3-[(isopropylamino)methyl]piperazine-1-carboxylate

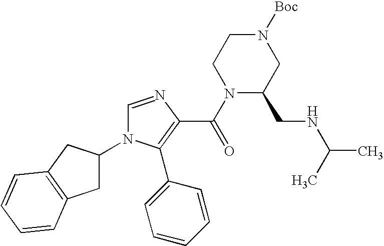

MS (ESI+, m/e) 544 (M+1)

### Reference Example 637

### tert-Butyl (3R)-3-{[(4-ethoxy-4-oxobutanoyl)(isopropyl)amino]methyl}-4-[(5-methyl-1,2-diphenyl-1H-pyrrol-3-yl)carbonyl]piperazine-1-carboxylate

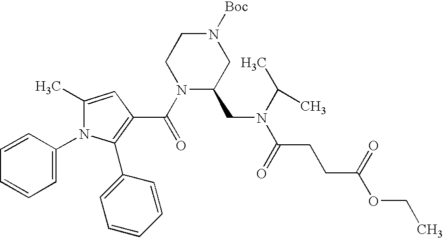

To a mixture of tert-butyl (3R)-3-[(isopropylamino)methyl]-4-[(5-methyl-1,2-diphenyl-1H-pyrrol-3-yl)carbonyl]piperazine-1-carboxylate (390 mg), pyridine (119 mg) and THF (6 ml) was added ethylsuccinyl chloride (248 mg) and, after stirring at room temperature for 15 hr, the mixture was poured into water, and extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo. The residue was subjected to silica gel column chromatography, and the fraction eluted with ethyl acetate-hexane (1:2 to 1:0) was concentrated in vacuo to give the desired product (441 mg) as an amorphous solid.

MS (ESI+, m/e) 645 (M+1)

In the same manner as in Reference Example 637, the following compound (Reference Example 638) was obtained.

### Reference Example 638

### tert-Butyl (3R)-3-{[(5-ethoxy-5-oxopentanoyl)(isopropyl)amino]methyl}-4-[(5-methyl-1,2-diphenyl-1H-pyrrol-3-yl)carbonyl]piperazine-1-carboxylate

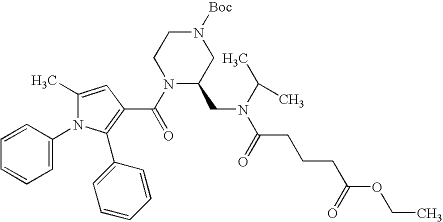

MS (ESI+, m/e) 659 (M+1)

### Reference Example 639

### tert-Butyl (3R)-3-{[{[(3-ethoxy-3-oxopropyl)amino]carbonyl}(isopropyl)amino]methyl}-4-[(5-methyl-1,2-diphenyl-1H-pyrrol-3-yl)carbonyl]piperazine-1-carboxylate

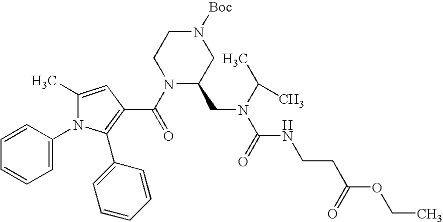

To a mixture of tert-butyl (3R)-3-[(isopropylamino)methyl]-4-[(5-methyl-1,2-diphenyl-1H-pyrrol-3-yl)carbonyl]piperazine-1-carboxylate (390 mg), DMAP (184 mg) and THF (6 ml) was added ethyl 3-isocyanatopropionate (216 mg). After stirring at room temperature for 5 hr, the mixture was poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated, in vacuo. The residue was subjected to silica gel column chromatography, and the fraction eluted with ethyl acetate-hexane (1:2 to 1:0) was concentrated in vacuo to give the desired product (458 mg) as an amorphous solid.

MS (ESI+, m/e) 660 (M+1)

### Reference Example 640

### 4-[({(2R)-4-(tert-Butoxycarbonyl)-1-[(5-methyl-1,2-diphenyl-1H-pyrrol-3-yl)carbonyl]piperazin-2-yl}methyl)(isopropyl)amino]-4-oxobutyric acid

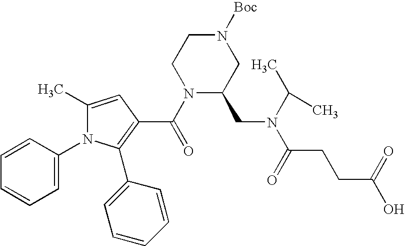

tert-Butyl (3R)-3-{[(4-ethoxy-4-oxobutanoyl)(isopropyl)amino]methyl}-4-[(5-methyl-1,2-diphenyl-1H-pyrrol-3-yl)carbonyl]piperazine-1-carboxylate (349 mg) was dissolved in ethanol (9 ml), and a 2 N aqueous lithium hydroxide solution (6 ml) was added. After stirring at room temperature for 1 hr, the mixture was poured into a 10% aqueous citric acid solution, and the mixture was extracted with ethyl acetate. The extract was washed successively with water and brine, and dried over anhydrous magnesium sulfate. The solvent was evaporated in vacuo to give the desired product (333 mg) as an amorphous solid.

MS (ESI+, m/e) 617 (M+1)

In the same manner as in Reference Example 640, the following compounds (Reference Examples 641 and 642) were obtained.

### Reference Example 641

### 5-[({(2R)-4-(tert-Butoxycarbonyl)-1-[(5-methyl-1,2-diphenyl-1H-pyrrol-3-yl)carbonyl]piperazin-2-yl}methyl)(isopropyl)amino]-5-oxopentanoic acid

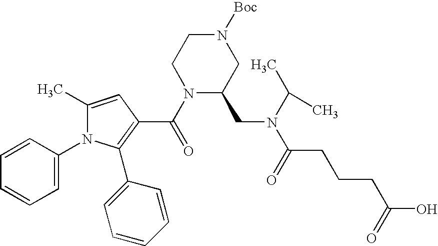

MS (ESI+, m/e) 631 (M+1)

### Reference Example 642

### N-{[({(2R)-4-(tert-Butoxycarbonyl)-1-[(5-methyl-1,2-diphenyl-1H-pyrrol-3-yl)carbonyl)piperazin-2-yl}methyl)(isopropyl)amino]carbonyl}-β-alanine

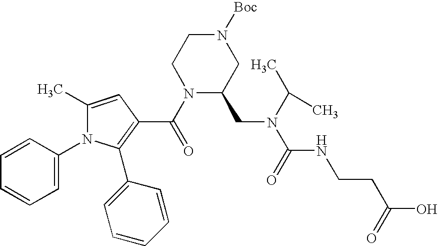

MS (ESI+, m/e) 632 (M+1)

### Reference Example 643

### 1-({(2R)-4-(tert-Butoxycarbonyl)-1-[(5-methyl-1,2-diphenyl-1H-pyrrol-3-yl)carbonyl]piperazin-2-yl}methyl)piperidine-4-carboxylic acid

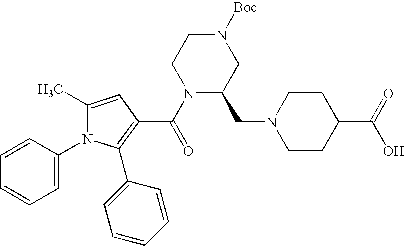

tert-Butyl (3R)-3-{[4-(ethoxycarbonyl)piperidin-1-yl]methyl}-4-[(5-methyl-1,2-diphenyl-1H-pyrrol-3-yl)carbonyl]piperazine-1-carboxylate (665 mg) was dissolved in ethanol (18 ml), and a 2 N aqueous lithium hydroxide solution (12 ml) was added. After stirring at room temperature for 1 hr, the reaction mixture was ice-cooled, neutralized with 1 N hydrochloric acid, and poured into brine, and the mixture was extracted with ethyl acetate. The extract was washed with brine, and dried over anhydrous magnesium sulfate. The solvent was evaporated in vacuo to give the desired product (634 mg) as an amorphous solid.

MS (ESI+, m/e) 587 (M+1)

### Reference Example 644

### tert-Butyl (3R)-3-{[(4-amino-4-oxobutanoyl)(isopropyl)amino]methyl)-4-[(5-methyl-1,2-diphenyl-1H-pyrrol-3-yl)carbonyl]piperazine-1-carboxylate

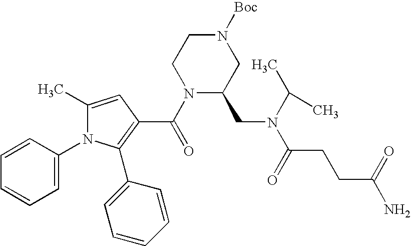

A mixture of 4-[({(2R)-4-(tert-butoxycarbonyl)-1-[(5-methyl-1,2-diphenyl-1H-pyrrol-3-yl)carbonyl)piperazin-2-yl}methyl)(isopropyl)amino]-4-oxobutyric acid (202 mg), HOBt ammonium salt (60 mg), WSC.HCl (75 mg) and DMF (2 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo to give the desired product (185 mg) as an amorphous solid.

MS (ESI+, m/e) 616 (M+1)

In the same manner as in Reference Example 644, the following compounds (Reference Examples 645 and 646) were obtained.

### Reference Example 645

### tert-Butyl (3R)-3-{[(5-amino-5-oxopentanoyl)(isopropyl)amino]methyl}-4-[(5-methyl-1,2-diphenyl-1H-pyrrol-3-yl)carbonyl]piperazine-1-carboxylate

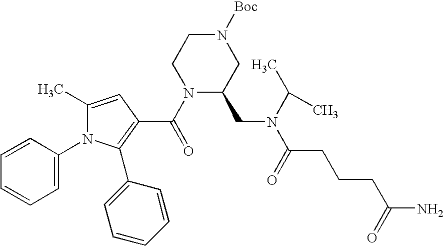

MS (ESI+, m/e) 630 (M+1)

### Reference Example 646

### tert-Butyl (3R)-3-{[{[(3-amino-3-oxopropyl)amino]carbonyl}(isopropyl)amino]methyl}-4-[(5-methyl-1,2-diphenyl-1H-pyrrol-3-yl)carbonyl]piperazine-1-carboxylate

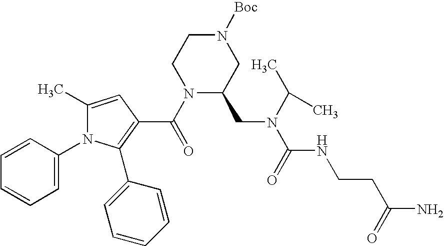

MS (ESI+, m/e) 631 (M+1)

### Reference Example 647

### tert-Butyl (3R)-3-{[4-(aminocarbonyl)piperidin-1-yl]methyl}-4-[(5-methyl-1,2-diphenyl-1H-pyrrol-3-yl)carbonyl]piperazine-1-carboxylate

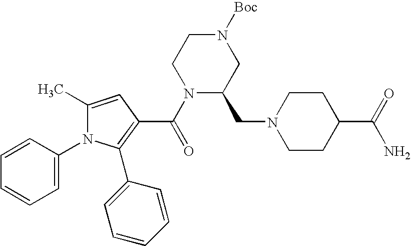

A mixture of 1-({(2R)-4-(tert-butoxycarbonyl)-1-[(5-methyl-1,2-diphenyl-1H-pyrrol-3-yl)carbonyl]piperazin-2-yl}methyl)piperidine-4-carboxylic acid (160 mg), HOBt ammonium salt (50 mg), WSC.HCl (63 mg) and DMF (1.8 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-methanol (1:0 to 20:1) was concentrated in vacuo to give the desired product (139 mg) as an amorphous solid.

MS (ESI+, m/e) 586 (M+1)

### Reference Example 648

### tert-Butyl (3R)-3-[(4-{[(4-hydroxybutyl)amino]carbonyl}piperidin-1-yl)methyl]-4-[(5-methyl-1,2-diphenyl-1H-pyrrol-3-yl)carbonyl]piperazine-1-carboxylate

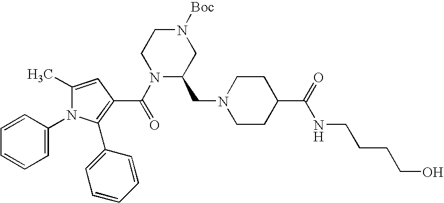

A mixture of 1-({(2R)-4-(tert-butoxycarbonyl)-1-[(5-methyl-1,2-diphenyl-1H-pyrrol-3-yl)carbonyl]piperazin-2-yl}methyl)piperidine-4-carboxylic acid (160 mg), 4-amino-1-butanol (27 mg), WSC.HCl (63 mg), HOBt (44 mg) and DMF (1.8 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-methanol (1:0 to 20:1) was concentrated in vacuo to give the desired product (149 mg) as an amorphous solid.

MS (ESI+, m/e) 658 (M+1)

### Reference Example 649

### tert-Butyl (3R)-3-[(4-{[(3-amino-3-oxopropyl)amino]carbonyl}piperidin-1-yl)methyl]-4-[(5-methyl-1,2-diphenyl-1H-pyrrol-3-yl)carbonyl]piperazine-1-carboxylate

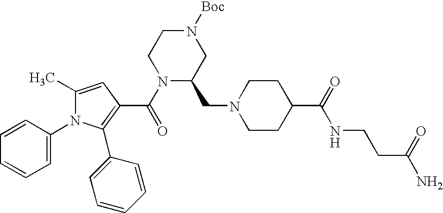

A mixture of 1-({(2R)-4-(tert-butoxycarbonyl)-1-[(5-methyl-1,2-diphenyl-1H-pyrrol-3-yl)carbonyl]piperazin-2-yl}methyl)piperidine-4-carboxylic acid (160 mg), β-alaninamide hydrochloride (27 mg), WSC.HCl (63 mg), HOBt (44 mg), triethylamine (33 mg) and DMF (1.8 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-methanol (1:0 to 20:1) was concentrated in vacuo to give the desired product (144 mg) as an amorphous solid.

MS (ESI+, m/e) 657 (M+1)

### Reference Example 650

### tert-Butyl (3R)-3-{[(4-amino-4-oxobutanoyl)(isopropyl)amino]methyl)-4-{[1-(2,3-dihydro-1H-inden-2-yl)-5-phenyl-1H-imidazol-4-yl]carbonyl}piperazine-1-carboxylate

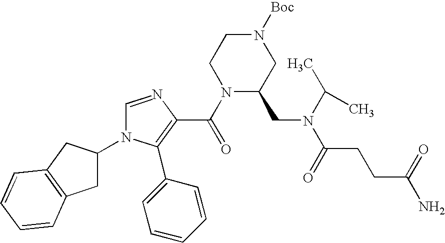

A mixture of tert-butyl (3R)-4-{[1-(2,3-dihydro-1H-inden-2-yl)-5-phenyl-1H-imidazol-4-yl]carbonyl}-3-[(isopropylamino)methyl]piperazine-1-carboxylate (524 mg), succinic acid monoamide (226 mg), WSC.HCl (370 mg), HOBt (260 mg) and DMF (10 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo. The residue was subjected to basic silica gel column chromatography, and the fraction eluted with ethyl acetate-chloroform-methanol (1:0:0 to 10:10:1) was concentrated in vacuo to give the desired product (65 mg) as an amorphous solid.

MS (ESI+, m/e) 643 (M+1)

### Reference Example 651

### tert-Butyl (3S)-4-[(5-methyl-1,2-diphenyl-1H-pyrrol-3-yl)carbonyl]-3-[(2-oxo-1,3-oxazolidin-3-yl)methyl]piperazine-1-carboxylate

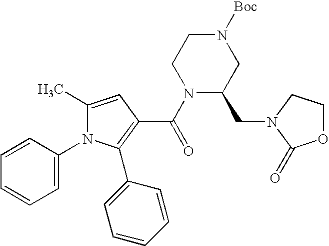

tert-Butyl (3R)-3-{[(2-hydroxyethyl)amino]methyl}-4-[(5-methyl-1,2-diphenyl-1H-pyrrol-3-yl)carbonyl]piperazine-1-carboxylate (238 mg) and ethyl trichloroacetate (176 mg) were dissolved in 1,2-dichloroethane (0.5 ml), and the mixture was stirred under argon atmosphere at 100° C. for 3.5 hr. The reaction mixture was cooled to room temperature, and subjected to silica gel column chromatography. The fraction eluted with ethyl acetate-hexane (1:1 to 1:0) was concentrated in vacuo, and the crystals were collected by filtration to give the desired product (192 mg).

MS (ESI+, m/e) 545 (M+1)

### Reference Example 652

### tert-Butyl (3R)-4-[(5-methyl-1,2-diphenyl-1H-pyrrol-3-yl)carbonyl]-3-[(3-oxomorpholino)methyl]piperazine-1-carboxylate

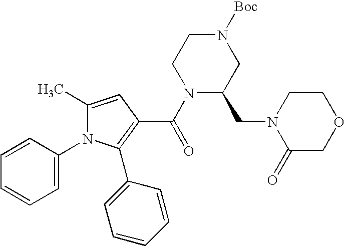

tert-Butyl (3R)-3-{[(2-hydroxyethyl)amino]methyl}-4-[(5-methyl-1,2-diphenyl-1H-pyrrol-3-yl)carbonyl]piperazine-1-carboxylate (238 mg) and pyridine (54 mg) were dissolved in THF (2 ml), chloroacetyl chloride (78 mg) was added. After stirring at room temperature for 1.5 hr, the reaction mixture was poured into saturated aqueous sodium bicarbonate solution, and extracted with ethyl acetate-THF (2:1). The extract was washed with brine, and dried over anhydrous magnesium sulfate. The solvent was evaporated in vacuo, and the crystals were collected by filtration. The obtained crystals were dissolved in methanol-dichloromethane (3:2, 5 ml), pulverized potassium hydroxide (100 mg) was added, and the mixture was stirred at room temperature for 15 hr. The reaction mixture was poured into a 10% aqueous citric acid solution, and extracted with ethyl acetate-THF (3:1). The extract was washed successively with saturated aqueous sodium bicarbonate solution and brine, and dried over anhydrous magnesium sulfate. The solvent was evaporated in vacuo, and the crystals were collected by filtration to give the desired product (139 mg).

MS (ESI+, m/e) 559 (M+1)

### Reference Example 653

### {4-(tert-Butoxycarbonyl)-1-[(5-methyl-1,2-diphenyl-1H-pyrrol-3-yl)carbonyl]piperazin-2-yl}acetic acid

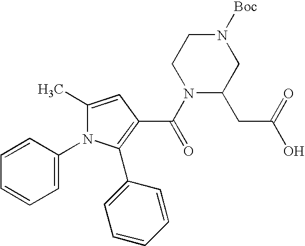

tert-Butyl 3-(2-methoxy-2-oxoethyl)-4-[(5-methyl-1,2-diphenyl-1H-pyrrol-3-yl)carbonyl]piperazine-1-carboxylate (2.76 g) was dissolved in THF-methanol (1:1, 80 ml), a 2 N aqueous lithium hydroxide solution (56 ml) was added. After stirring at room temperature for 1 hr, the reaction mixture was poured into water, and the mixture was weakly acidified (pH 3) with 1 N hydrochloric acid with vigorous stirring, and the mixture was extracted with ethyl acetate. The extract was washed successively with water and brine, and dried over anhydrous magnesium sulfate. The solvent was evaporated in vacuo to give the desired product (2.62 g) as an amorphous solid.

MS (ESI+, m/e) 504 (M+1)

### Reference Example 654

### {4-Benzyl-1-[(5-methyl-1,2-diphenyl-1H-pyrrol-3-yl)carbonyl]piperazin-2-yl}acetic acid

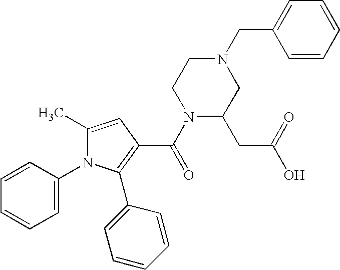

Methyl {4-benzyl-1-[(5-methyl-1,2-diphenyl-1H-pyrrol-3-yl)carbonyl]piperazin-2-yl}acetate (3.39 g) was dissolved in methanol (80 ml), and a 2 N aqueous lithium hydroxide solution (70 ml) was added. After stirring at room temperature for 1.5 hr, the reaction mixture was poured into water, and neutralized with concentrated hydrochloric acid. The mixture was saturated with sodium chloride, and extracted with ethyl acetate. The extract was washed with brine, and dried over anhydrous magnesium sulfate. The solvent was evaporated in vacuo to give the desired product (3.22 g) as an amorphous solid.

MS (ESI+, m/e) 494 (M+1)

### Reference Example 655

### tert-Butyl 3-(2-amino-2-oxoethyl)-4-[(5-methyl-1,2-diphenyl-1H-pyrrol-3-yl)carbonyl]piperazine-1-carboxylate

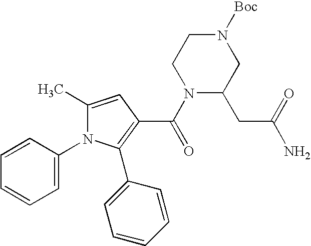

A solution of {4-(tert-butoxycarbonyl)-1-[(5-methyl-1,2-diphenyl-1H-pyrrol-3-yl)carbonyl]piperazin-2-yl}acetic acid (252 mg), HOBt ammonium salt (91 mg), WSC.HCl (115 mg) and DMF (3.5 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo to give the desired product (236 mg) as an amorphous solid.

MS (ESI+, m/e) 503 (M+1)

### Reference Example 656

### 2-{4-Benzyl-1-[(5-methyl-1,2-diphenyl-1H-pyrrol-3-yl)carbonyl]piperazin-2-yl}acetamide

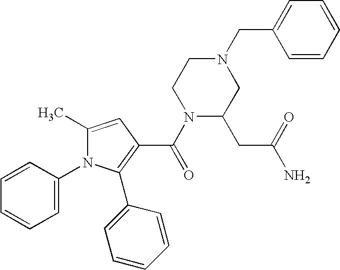

A solution of {4-benzyl-1-[(5-methyl-1,2-diphenyl-1H-pyrrol-3-yl)carbonyl]piperazin-2-yl}acetic acid (954 mg), HOBt ammonium salt (353 mg), WSC.HCl (445 mg) and DMF (12 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo to give the desired product (899 mg) as an amorphous solid.

MS (ESI+, m/e) 493 (M+1)

### Reference Example 657

### tert-Butyl 3-(2-anilino-2-oxoethyl)-4-[(5-methyl-1,2-diphenyl-1H-pyrrol-3-yl)carbonyl]piperazine-1-carboxylate

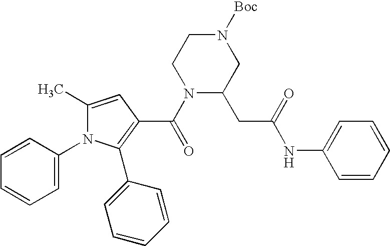

A solution of {4-(tert-butoxycarbonyl)-1-[(5-methyl-1,2-diphenyl-1H-pyrrol-3-yl)carbonyl]piperazin-2-yl}acetic acid (252 mg), aniline (51 mg), WSC.HCl (115 mg), HOBt (81 mg) and DMF (3.5 ml) was stirred at room temperature for 15 hr, and poured into water, and the mixture was extracted with ethyl acetate. The extract was washed ‘successively with a 10% aqueous citric acid solution, water, saturated aqueous sodium bicarbonate solution, water and brine, and dried over anhydrous magnesium sulfate. The solvent was evaporated in vacuo to give the desired product (270 mg) as an amorphous solid.

MS (ESI+, m/e) 579 (M+1)

In the same manner as in Reference Example 657, the following compound (Reference Example 658) was obtained.

### Reference Example 658

### tert-Butyl 3-[2-(isopropylamino)-2-oxoethyl]-4-[(5-methyl-1,2-diphenyl-1H-pyrrol-3-yl)carbonyl]piperazine-1-carboxylate

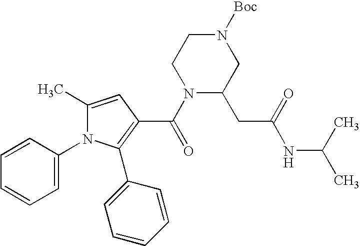

MS (ESI+, m/e) 545 (M+1)

### Reference Example 659

### tert-Butyl 4-[(5-methyl-1,2-diphenyl-1H-pyrrol-3-yl)carbonyl]-3-[(5-phenyl-1,3,4-oxadiazol-2-yl)methyl]piperazine-1-carboxylate

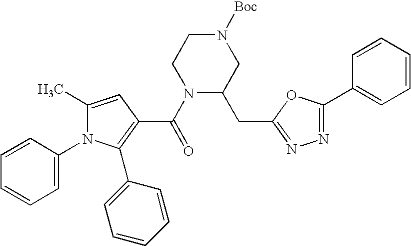

A mixture of {4-(tert-butoxycarbonyl)-1-[(5-methyl-1,2-diphenyl-1H-pyrrol-3-yl)carbonyl]piperazin-2-yl}acetic acid (353 mg), 5-phenyltetrazole (113 mg), DCC (159 mg) and toluene (4 ml) was stirred at 100° C. for 2.5 hr, and concentrated in vacuo, and the residue was diluted with ethyl acetate-diethyl ether (1:1, about 10 ml). The insoluble material was filtered off and the filtrate was concentrated in vacuo. The residue was subjected to basic silica gel column chromatography, and the fraction eluted with ethyl acetate-hexane (1:1 to 1:0) was concentrated in vacuo to give the desired product (396 mg) as an amorphous solid.

MS (ESI+, m/e) 604 (M+1)

### Reference Example 660

### tert-Butyl 4-[(5-methyl-1,2-diphenyl-1H-pyrrol-3-yl)carbonyl]-3-[(5-methyl-1,3,4-oxadiazol-2-yl)methyl]piperazine-1-carboxylate

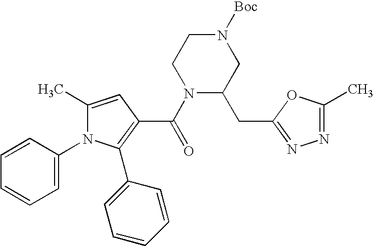

A mixture of {4-(tert-butoxycarbonyl)-1-[(5-methyl-1,2-diphenyl-1H-pyrrol-3-yl)carbonyl]piperazin-2-yl}acetic acid (353 mg), 5-methyltetrazole (65 mg), DCC (159 mg) and toluene (4 ml) was stirred at 100° C. for 16 hr, and concentrated in vacuo, and the residue was diluted with ethyl acetate-diethyl ether (1:1, about 10 ml). The insoluble material was filtered off and the filtrate was concentrated in vacuo. The residue was subjected to basic silica gel column chromatography, and the fraction eluted with ethyl acetate-hexane (1:2 to 1:0) was concentrated in vacuo, and the, crystals were collected by filtration to give the desired product (290 mg).

MS (ESI+, m/e) 542 (M+1)

### Reference Example 661

### 4-Benzyl-1-[(5-methyl-1,2-diphenyl-1H-pyrrol-3-yl)carbonyl]-2-[(4-methyl-1,3-oxazol-2-yl)methyl]piperazine

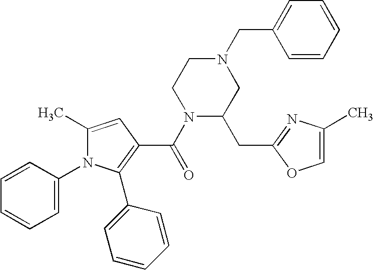

2-{4-Benzyl-1-[(5-methyl-1,2-diphenyl-1H-pyrrol-3-yl)carbonyl]piperazin-2-yl}acetamide (482 mg) was dissolved in chloroacetone (2 ml) and stirred at 120° C. for 3.5 hr. The mixture was returned to room temperature, diluted with saturated aqueous sodium bicarbonate solution, and extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:2 to 1:0) was concentrated in vacuo to give the desired product (88 mg) as an oil.

MS (ESI+, m/e) 531 (M+1)

### Reference Example 662

### 2-{4-Benzyl-1-[(5-methyl-1,2-diphenyl-1H-pyrrol-3-yl)carbonyl]piperazin-2-yl}ethanol

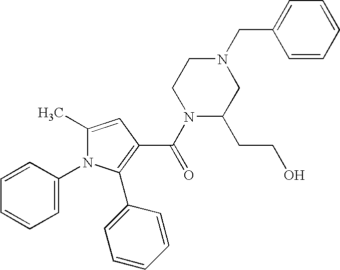

Sodium borohydride (7.23 g) was suspended in THF-ethanol (1:1, 160 ml) and the suspension was ice-cooled. Pulverized calcium chloride (10.60 g) was added by small portions over 5 min. After stirring at 0° C. for 30 min, a solution of methyl {4-benzyl-1-[(5-methyl-1,2-diphenyl-1H-pyrrol-3-yl)carbonyl]piperazin-2-yl}acetate (6.06 g) in THF (30 ml) was added dropwise over 10 min, and the mixture was stirred at 0° C. for 1.5 hr, and at room temperature for 2.5 hr. Ethyl acetate (100 ml) was added dropwise to the reaction mixture, and the mixture was poured into ice-water, vigorously stirred at room temperature for 20 min and extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (4:1 to 1:0) was concentrated in vacuo to give the desired product (5.49 g) as an amorphous solid.

MS (ESI+, m/e) 480 (M+1)

### Reference Example 663

### {4-Benzyl-1-[(5-methyl-1,2-diphenyl-1H-pyrrol-3-yl)carbonyl]piperazin-2-yl}acetoaldehyde

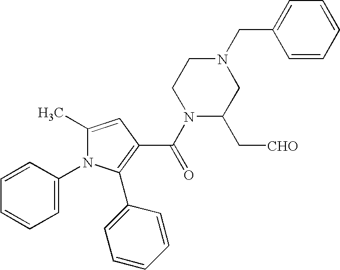

2-{4-Benzyl-1-[(5-methyl-1,2-diphenyl-1H-pyrrol-3-yl)carbonyl]piperazin-2-yl}ethanol (2.74 g) was dissolved in dichloromethane (30 ml), and a solution of pyridine sulfur trioxide complex (2.73 g) in DMSO (30 ml) and triethylamine (1.73 g) were added at 0° C. The reaction mixture was stirred at 0° C. for 2 hr, and poured into ice-cooled saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:1 to 2:1) was concentrated in vacuo, and the crystals were collected by filtration to give the desired product (1.21 g).

MS (ESI+, m/e) 478 (M+1)

### Reference Example 664

### N-(2-{4-Benzyl-1-[(5-methyl-1,2-diphenyl-1H-pyrrol-3-yl)carbonyl]piperazin-2-yl}ethyl)propan-2-amine

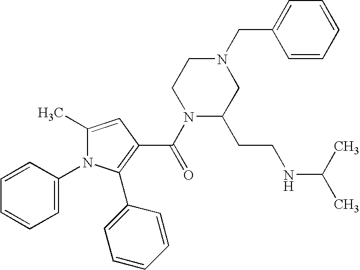

A solution of (4-benzyl-1-[(5-methyl-1,2-diphenyl-1H-pyrrol-3-yl)carbonyl]piperazin-2-yl}acetoaldehyde (573 mg), isopropylamine (142 mg), acetic acid (144 mg), dichloromethane (5 ml) and DMF (2.5 ml) was stirred at room temperature for 40 min, sodium triacetoxyborohydride (509 mg) was added and the mixture was stirred at room temperature for additional 15 hr. The reaction mixture was poured into saturated aqueous sodium bicarbonate solution, and the mixture was extracted with ethyl acetate. The extract was washed successively with water and brine, and dried over anhydrous magnesium sulfate. The solvent was evaporated in vacuo, and the crystals were collected by filtration to give the desired product (397 mg).

MS (ESI+, m/e) 521 (M+1)

### Reference Example 665

### Ethyl 4-[(2-{4-benzyl-1-[(5-methyl-1,2-diphenyl-1H-pyrrol-3-yl)carbonyl]piperazin-2-yl}ethyl)(isopropyl)amino]-4-oxobutyrate

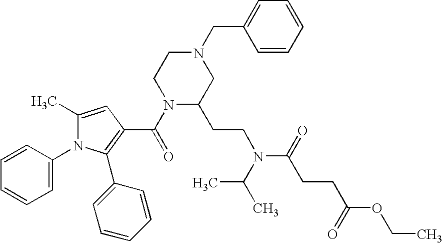

To a mixture of N-(2-{4-benzyl-1-[(5-methyl-1,2-diphenyl-1H-pyrrol-3-yl)carbonyl]piperazin-2-yl}ethyl)propan-2-amine (446 mg), triethylamine (173 mg) and THF (7 ml) was added ethylsuccinyl chloride (282 mg) and, after stirring at room temperature for 3 days, and the mixture was poured into saturated aqueous sodium bicarbonate solution, and extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:2 to 1:0) was concentrated in vacuo to give the desired product (478 mg) as an oil.

MS (ESI+, m/e) 649 (M+1)

### Reference Example 666

### 4-[(2-{4-Benzyl-1-[(5-methyl-1,2-diphenyl-1H-pyrrol-3-yl)carbonyl]piperazin-2-yl}ethyl)(isopropyl)amino]-4-oxobutyric acid

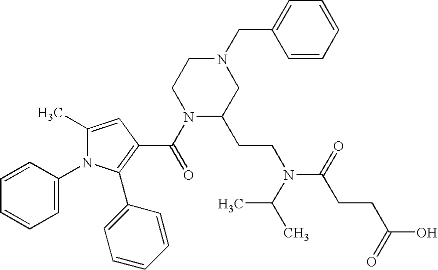

Ethyl 4-[(2-{4-benzyl-1-[(5-methyl-1,2-diphenyl-1H-pyrrol-3-yl)carbonyl]piperazin-2-yl}ethyl)(isopropyl)amino]-4-oxobutyrate (478 mg) was dissolved in ethanol (12 ml), and a 2 N aqueous lithium hydroxide solution (8 ml) was added. After stirring at room temperature for 1 hr, the mixture was poured into water, and neutralized with 6 N hydrochloric acid. The mixture was saturated with sodium chloride, and extracted with ethyl acetate. The extract was washed with brine, and dried over anhydrous magnesium sulfate. The solvent was evaporated in vacuo to give the desired product (455 mg) as an amorphous solid.

MS (ESI+, m/e) 621 (M+1)

### Reference Example 667

### N-(2-{4-Benzyl-1-[(5-methyl-1,2-diphenyl-1H-pyrrol-3-yl)carbonyl]piperazin-2-yl}ethyl)-N-isopropylsuccinamide

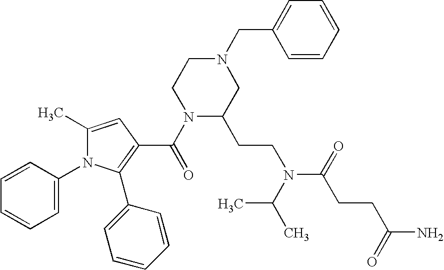

A mixture of 4-[(2-{4-benzyl-1-[(5-methyl-1,2-diphenyl-1H-pyrrol-3-yl)carbonyl]piperazin-2-yl}ethyl)(isopropyl)amino]-4-oxobutyric acid (267 mg), HOBt ammonium salt (79 mg), WSC.HCl (99 mg) and DMF (3 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo to give the desired product (234 mg) as an amorphous solid.

MS (ESI+, m/e) 620 (M+1)

### Reference Example 668

### N-(2-Aminophenyl)-2-{4-benzyl-1-[(5-methyl-1,2-diphenyl-1H-pyrrol-3-yl)carbonyl]piperazin-2-yl}acetamide

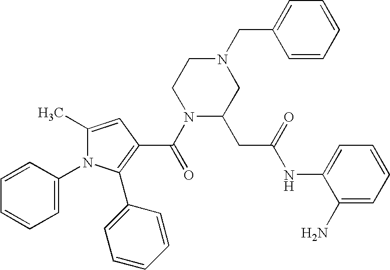

A solution of {4-benzyl-1-[(5-methyl-1,2-diphenyl-1H-pyrrol-3-yl)carbonyl]piperazin-2-yl}acetic acid (484 mg), o-phenylenediamine (530 mg), WSC.HCl (376 mg), HOBt (265 mg) and DMF (10 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane-methanol (1:1:0 to 20:0:1) was concentrated in vacuo to give the desired product (559 mg) as an amorphous solid.

MS (ESI+, m/e) 584 (M+1).

### Reference Example 669

### 2-({4-Benzyl-1-[(5-methyl-1,2-diphenyl-1H-pyrrol-3-yl)carbonyl]piperazin-2-yl}methyl)-1H-benzimidazole

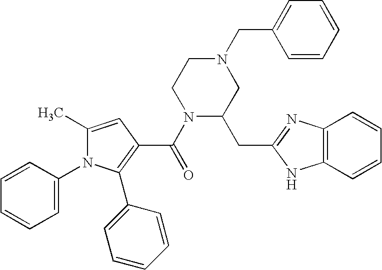

N-(2-Aminophenyl)-2-{4-benzyl-1-[(5-methyl-1,2-diphenyl-1H-pyrrol-3-yl)carbonyl]piperazin-2-yl}acetamide (559 mg) was dissolved in acetic acid (15 ml) and, after stirring at 65° C. for 2 hr, the mixture was concentrated in vacuo. The residue was diluted by adding saturated aqueous sodium bicarbonate solution by small portions, and the mixture was extracted with ethyl acetate. The extract was washed successively with water and brine, and dried over anhydrous magnesium sulfate. The solvent was evaporated in vacuo, and the crystals were collected by filtration to give the desired product (453 mg).

MS (ESI+, m/e) 566 (M+1)

### Reference Example 670

### 4-Benzyl-1-[(5-methyl-1,2-diphenyl-1H-pyrrol-3-yl)carbonyl]-2-(2-phenoxyethyl)piperazine

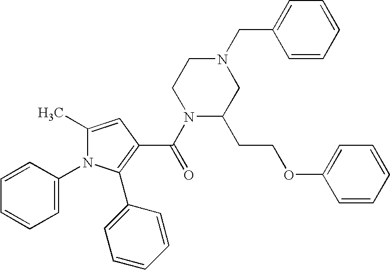

2-{4-Benzyl-1-[(5-methyl-1,2-diphenyl-1H-pyrrol-3-yl)carbonyl]piperazin-2-yl}ethanol (730 mg), phenol (215 mg) and triphenylphosphine (599 mg) were dissolved in toluene (15 ml), the mixture was ice-cooled, and DEAD (40% toluene solution) (1.04 ml) was added under argon atmosphere. After stirring at room temperature for 15 hr, the reaction mixture was poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to silica gel column chromatography, and the fraction eluted with ethyl acetate-hexane (1:2) was concentrated in vacuo. The insoluble material was filtered off, washed with diisopropyl ether-hexane, and the filtrate was concentrated in vacuo to give the desired product (191 mg) as an oil.

MS (ESI+, m/e) 556 (M+1)

### Reference Example 671

N-(2-(4-{[(2R)-2,4-Dibenzylpiperazin-1-yl]carbonyl}-5-phenyl-1H-imidazol-1-yl)ethyl]tetrahydro-2H-pyran-4-amine

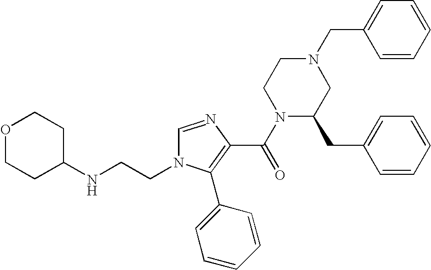

To solution of tert-butyl [2-(4-{[(2R)-2,4-dibenzylpiperazin-1-yl]carbonyl}-5-phenyl-1H-imidazol-1-yl)ethyl](tetrahydro-2H-pyran-4-yl)carbamate (5.43 g) in dichloromethane (2.5 ml) was added TFA (25 ml), and the mixture was stirred at room temperature for 40 min. The reaction mixture was poured into saturated aqueous sodium bicarbonate solution by small portions, and the mixture was basified by adding potassium carbonate by small portions, and the mixture was extracted with ethyl acetate. The extract was washed with brine, and dried over anhydrous magnesium sulfate. The solvent was evaporated in vacuo to give the desired product (4.36 g) as an amorphous solid.

MS (ESI+, m/e) 564 (M+1)

### Reference Example 672

### 4-(Acetylamino)-N-[2-(4-{[(2R)-2,4-dibenzylpiperazin-1-yl]carbonyl}-5-phenyl-1H-imidazol-1-yl)ethyl]-N-(tetrahydro-2H-pyran-4-yl)butanamide

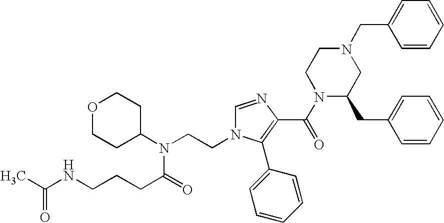

A solution of N-[2-(4-{[(2R)-2,4-dibenzylpiperazin-1-yl]carbonyl}-5-phenyl-1H-imidazol-1-yl)ethyl]tetrahydro-2H-pyran-4-amine (423 mg), 4-(acetylamino)butyric acid (120 mg), WSC.HCl (173 mg), HOBt (122 mg) and DMF (5 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane-methanol (1:1:0 to 20:0:1) was concentrated in vacuo to give the desired product (231 mg) as an amorphous solid.

MS (ESI+, m/e) 691 (M+1)

In the same manner as in Reference Example 672, the following compounds (Reference Examples 673 and 674) were obtained.

### Reference Example 673

### N-[2-(4-{[(2R)-2,4-Dibenzylpiperazin-1-yl]carbonyl}-5-phenyl-1H-imidazol-1-yl)ethyl]-2-(2-methoxyethoxy)-N-(tetrahydro-2H-pyran-4-yl)acetamide

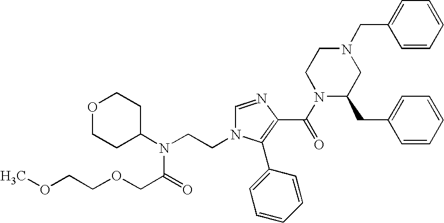

MS (ESI+, m/e) 680 (M+1)

### Reference Example 674

### N-[2-(4-{[(2R)-2,4-Dibenzylpiperazin-1-yl]carbonyl}-5-phenyl-1H-imidazol-1-yl)ethyl]-N-(tetrahydro-2H-pyran-4-yl)-4-(2-thienyl)butanamide

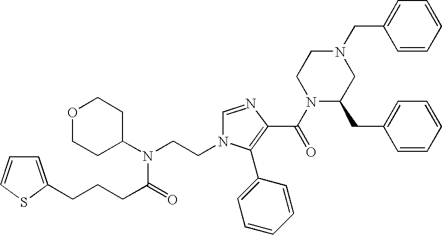

MS (ESI+, m/e) 716 (M+1)

### Reference Example 675

### Ethyl [(2R)-2,4-Dibenzylpiperazin-1-yl](oxo)acetate

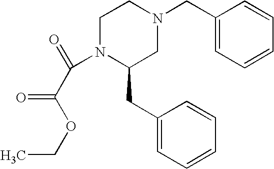

Ethyl chloro(oxo)acetate (4.2 ml) was added to a solution of (R)-1,3-dibenzylpiperazine (10.0 g) and triethylamine (10.5 ml) in dichloromethane (100 ml) at 0° C. After stirring at room temperature for 4 hr, water was added, and the mixture was extracted with dichloroethane. The extract was washed with brine, and dried over anhydrous magnesium sulfate. The solvent was evaporated in vacuo. The residue was subjected to silica gel column chromatography, and the target fraction was concentrated in vacuo to give the desired product (13.0 g).

¹H-NMR (DMSO-d₆) δ 1.13-1.28 (3H, m), 1.95-2.09 (2H, m), 2.64 (1H, dd), 2.87-2.99 (1H, m), 3.07-3.21 (1H, m), 3.29-3.37 (3H, m), 3.49 (1H, dd), 3.61 (2H, dd), 4.11-4.17 (1H, m), 4.19-4.26 (1H, m), 6.95 (1H, dd), 6.99-7.08 (1H, m), 7.10-7.22 (3H, m), 7.26-7.39 (5H, m)

### Reference Example 676

### 2-[(2R)-2,4-Dibenzylpiperazin-1-yl]-2-oxoacetohydrazide

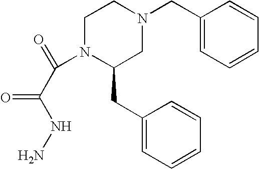

To a solution of ethyl [(2R)-2,4-dibenzylpiperazin-1-yl](oxo)acetate (18.0 g) in ethanol (180 ml) was added hydrazine monohydrate (5.24 ml), and the mixture was heated under reflux for 15 hr. The solvent was evaporated in vacuo, water was added to the residue, and the mixture was extracted with ethyl acetate. The extract was washed with brine, dried over anhydrous magnesium sulfate, and concentrated in vacuo to give the desired product (18.0 g).

¹H-NMR (DMSO-d₆) δ 1.83 (1H, td), 1.95-2.09 (1H, m), 2.55-2.70 (1H, m), 2.78 (1H, dd), 2.93 (1H, s), 3.21-3.31 (2H, m), 3.63 (2H, q), 3.87 (1H, s), 4.17 (1H, d), 4.34-4.50 (3H, m), 6.92-7.02 (2H, m), 7.09-7.18 (4H, m), 7.23-7.39 (4H, m)

### Reference Example 677

### (2R)-2,4-Benzyl-1-[(4,5-diphenyl-4H-1,2,4-triazol-3-yl)carbonyl]piperazine

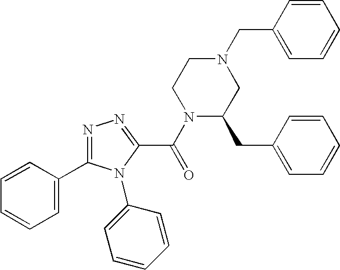

2-[(2R)-2,4-Dibenzylpiperazin-1-yl]-2-oxoacetohydrazide (1.0 g) and diphenylthioamide (0.64 g) were dissolved in n-butanol (20 ml), and the mixture was heated under reflux for 15 hr. The solvent was evaporated in vacuo, the residue was subjected to silica gel column chromatography, and the target fraction was concentrated in vacuo to give the desired product (0.19 g).

MS (ESI+, m/e) 514 (M+1)

### Reference Example 678

### (2R)-2,4-Dibenzyl-1-(methylsulfonyl)piperazine

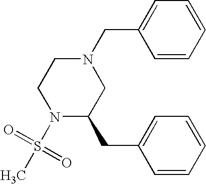

(3R)-1,3-Dibenzylpiperazine (7.99 g), triethylamine (3.64 g) and DMAP (367 mg) were dissolved in dichloromethane (120 ml), and methanesulfonyl chloride (3.78 g) was added dropwise at room temperature over 5 min. After stirring at room temperature for 1.5 hr, the reaction mixture was concentrated in vacuo, diluted with saturated aqueous sodium bicarbonate solution, and extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane (1:3 to 1:2) was concentrated in vacuo, and the crystals were collected by filtration to give the desired product (9.14 g).

¹H-NMR (CDCl₃) δ 2.10-2.21 (2H, m), 2.46 (3H, s), 2.72 (1H, d), 2.86 (1H, d), 3.09 (2H, d), 3.32-3.40 (2H, m), 3.53 (1H, d), 3.63-3.68 (1H, m), 4.08-4.12 (1H, m), 7.09-7.35 (10H, m)

MS (ESI+, m/e) 345 (M+1)

### Reference Example 679

### 2-{[(2R)-2,4-Dibenzylpiperazin-1-yl]sulfonyl}-1-phenylethanone

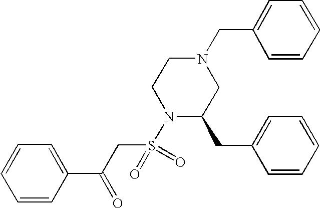

n-Butyllithium (1.6M hexane solution) (12.5 ml) was added to THF (25 ml) under argon atmosphere at −78° C., and a solution of (2R)-2,4-dibenzyl-1-(methylsulfonyl)piperazine (3.44 g) in THF (5 ml) was added dropwise over 5 min. After stirring at −78° C. for 15 min and at 0° C. for 10 min, the reaction mixture was cooled again to −78° C., and a solution of methyl benzoate (1.36 g) in THF (5 ml) was added dropwise over 5 min. After stirring at −78° C. for 1 hr and at 0° C. for 30 min, the reaction mixture was poured into 0.5 N hydrochloric acid, and the mixture was stirred at room temperature for 10 min. The mixture was carefully neutralized with saturated aqueous sodium bicarbonate solution, and extracted with ethyl acetate. The extract was washed with brine, and dried over anhydrous magnesium sulfate. The solvent was evaporated in vacuo. The residue was subjected to basic silica gel column chromatography, and the fraction eluted with ethyl acetate-hexane (1:2) was concentrated in vacuo to give the desired product (4.28 g) as an oil.

¹H-NMR (CDCl₃) δ 2.10-2.24 (2H, m), 2.69 (1H, d), 2.84 (1H, d), 3.04-3.20 (2H, m), 3.36-3.56 (3H, m), 3.70 (1H, d), 3.98 (1H, d), 4.06-4.10 (1H, m), 4.33 (1H, d), 7.04-7.19 (5H, m), 7.25-7.35 (5H, m), 7.45-7.50 (2H, m), 7.58-7.63 (1H, m), 7.87-7.91 (2H, m)

MS (ESI+, m/e) 449 (M+1)

### Reference Example 680

### (2R)-2,4-Dibenzyl-1-[(1,5-diphenyl-1H-pyrazol-4-yl)sulfonyl]piperazine

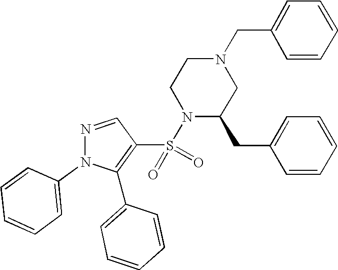

2-{[(2R)-2,4-Dibenzylpiperazin-1-yl]sulfonyl}-1-phenylethanone (800 mg) was dissolved in toluene (4 ml), N,N-dimethylformamide dimethylacetal (276 mg) was added at room temperature and the mixture was heated under reflux for 15 hr. The reaction mixture was concentrated in vacuo, and the residue was dissolved in ethanol (5 ml). Phenylhydrazine (193 mg) was added, and the mixture was stirred at room temperature for additional 4 hr. The reaction mixture was concentrated in vacuo, and the crystals were collected by filtration to give the desired product (649 mg).

¹H-NMR (CDCl₃) δ 1.31 (1H, dd), 1.51-1.61 (1H, m), 2.38 (1H, d), 2.57 (1H, d), 2.66 (1H, dd), 3.06 (1H, dt), 3.13-3.27 (3H, M), 3.32 (1H, d), 3.71 (1H, d), 6.89-6.92 (2H, m), 7.10-7.12 (3H, m), 7.15-7.37 (15H, m), 8.11 (1H, s)

MS (ESI+, m/e) 549 (M+1)

### Reference Example 681

### (2R)-2,4-Dibenzyl-1-[(1,5-diphenyl-1H-1,2,3-triazol-4-yl)sulfonyl]piperazine

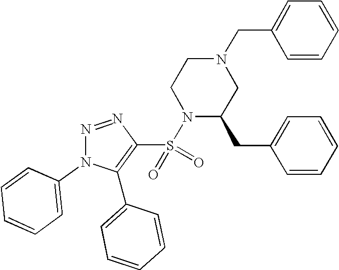

To a solution of 2-{[(2R)-2,4-dibenzylpiperazin-1-yl]sulfonyl)-1-phenylethanone (1.46 g), phenylazide (504 mg), methanol (25 ml) and THF (1 ml) was added sodium methoxide (28% methanol solution) (816 mg) at 0° C. and, after stirring at 60° C. for 18 hr, the mixture was poured into water and extracted with ethyl acetate-THF (3:1). The extract was washed successively with water and brine, and dried over anhydrous magnesium sulfate. The solvent was evaporated in vacuo, and the crystals were collected by filtration to give the desired product (508 mg).

¹H-NMR (CDCl₃) δ 1.99 (1H, dd), 2.15 (1H, dt), 2.57 (1H, d), 2.78 (1H, d), 2.84 (1H, dd), 3.25-3.35 (2H, m), 3.41-3.52 (2H, m), 3.76 (1H, d), 4.03-4.06 (1H, m), 6.92-6.95 (2H, m), 7.08-7.12 (3H, m), 7.22-7.44 (15H, m)

MS (ESI+, m/e) 550 (M+1)

### Examples

### Example 1 (Method A)

### (2R)-1-[(1,2-Diphenyl-1H-pyrrol-3-yl)carbonyl]-2-(2-phenylethyl)piperazine hydrochloride

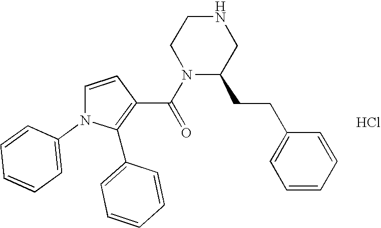

A solution of 1,2-diphenyl-1H-pyrrole-3-carboxylic acid (197 mg), (3R)-1-benzyl-3-(2-phenylethyl)piperazine (210 mg), WSC.HCl (172 mg), HOBt (121 mg) and DMF (5 ml) was stirred at room temperature for 15 hr. Then, the mixture was poured into a saturated aqueous sodium bicarbonate solution and extracted with ethyl acetate. The extract was washed successively with water and brine and dried over anhydrous magnesium sulfate, s and then the solvent was evaporated in vacuo. The residue was subjected to basic silica gel column chromatography, and the fraction eluted with ethyl acetate-hexane (1:2) was concentrated in vacuo. The resulting oil (355 mg) was dissolved in methanol (10 ml). Concentrated hydrochloric acid (60 μl) and 10% palladium on carbon (containing 50% water, 180 mg) were added thereto, and the mixture was subjected to catalytic hydrogenation at room temperature and atmospheric pressure for 15 hr. The catalyst was filtered off, and the filtrate was concentrated in vacuo. The residue was diluted is with a saturated aqueous sodium bicarbonate solution and extracted with ethyl acetate. The extract was washed successively with water and brine and dried over anhydrous magnesium sulfate, and then the solvent was evaporated in vacuo. To the residue were added diethyl ether (3 ml) and a 4 N hydrogen chloride-ethyl acetate solution (170 μl), and the crystals were collected by filtration to give the desired product (207 mg).

MS (ESI+, m/e) 436 (M+1)

### Example 2 (Method B)

### (2R)-2-Benzyl-1-[(1,2-diphenyl-1H-pyrrol-3-yl)carbonyl]piperazine hydrochloride

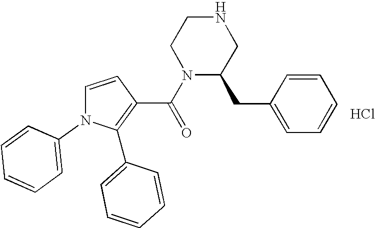

A solution of 1,2-diphenyl-1H-pyrrole-3-carboxylic acid (94 mg), (3R)-1,3-dibenzylpiperazine (95 mg), WSC.HCl (89 mg), HOBt (71 mg) and DMF (3 ml) was stirred at room temperature for 12 hr. Then, the mixture was poured into a saturated aqueous sodium bicarbonate solution and extracted with ethyl acetate. The extract was washed successively with water and brine and dried over anhydrous sodium sulfate, and then the solvent was evaporated in vacuo. The residue was subjected to silica gel column chromatography, and the target fraction was concentrated in vacuo to give an amorphous solid (130 mg). A portion thereof (120 mg) was dissolved in methanol (2 ml), 20% palladium on carbon hydroxide (containing 50% water, 50 mg) was added thereto, and the mixture was subjected to catalytic hydrogenation at room temperature and atmospheric pressure for 12 hr. The catalyst was filtered off, and the filtrate was concentrated in vacuo. The residue was dissolved in ethyl acetate, acidified with a 4 N hydrogen chloride-ethyl acetate solution, and then concentrated in vacuo to give the desired product (90 mg) as an amorphous solid.

MS (ESI+, m/e) 422 (M+1)

### Example 3 (Method C)

### (2R)-1-[(5-Methyl-1,2-diphenyl-1H-pyrrol-3-yl)carbonyl]-2-(2-thienylmethyl)piperazine

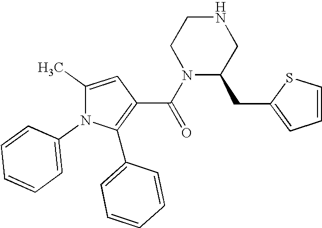

A solution of 5-methyl-1,2-diphenyl-1H-pyrrole-3-carboxylic acid (208 mg), (3R)-1-benzyl-3-(2-thienylmethyl)piperazine (204 mg), WSC.HCl (173 mg), HOBt (122 mg) and DMF (5 ml) was stirred at room temperature for 15 hr. Then, the mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to basic silica gel column chromatography, and the fraction eluted with ethyl acetate-hexane (1:2) was concentrated in vacuo. The resulting amorphous (336 mg) was dissolved in 1,2-dichloroethane (10 ml). To the resulting solution was added 1-chloroethyl chloroformate (136 mg) and stirred at room temperature for 15 min, and then the mixture was heated under reflux for 1 hr. The solvent was evaporated in vacuo, methanol (10 ml) was added to the residue, and the mixture was heated under reflux for 1 hr. Then, the mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to basic silica gel column chromatography, the fraction eluted with ethyl acetate-hexane-methanol (1:1:0 to 40:0:1) was concentrated in vacuo, and the crystals were collected by filtration to give the desired product (96 mg).

MS (ESI+, m/e) 442 (M+1)

### Example 4 (Method D)

### 2-Benzyl-1-[(1-benzyl-2-methyl-1H-pyrrol-3-yl)carbonyl]piperazine hydrochloride

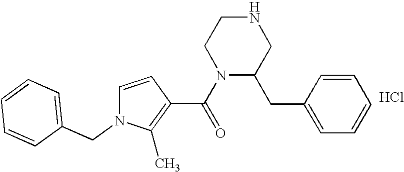

A solution of 1-benzyl-2-methyl-1H-pyrrole-3-carboxylic acid (150 mg), tert-butyl 3-benzylpiperazine-1-carboxylate (193 mg), WSC.HCl (174 mg), HOBt (139 mg) and DMF (10 ml) was stirred at room temperature for 12 hr. Then, the mixture was poured into a saturated aqueous sodium bicarbonate solution and extracted with ethyl acetate. The extract was washed successively with water and brine and dried over anhydrous sodium sulfate, and then the solvent was evaporated in vacuo. The residue was subjected to silica gel column chromatography, and the fraction eluted with ethyl acetate-hexane (1:9 to 1:3) was concentrated in vacuo to give an amorphous solid (140 mg). 110 mg of the resulting amorphous was dissolved in dichloromethane (2 ml), and TFA (2 ml) was added thereto. After stirring at room temperature for 2 hr, the mixture was poured into a saturated aqueous sodium bicarbonate solution (50 ml) and extracted with ethyl acetate. The extract was washed with brine and dried over anhydrous sodium sulfate, and then the solvent was evaporated in vacuo. The residue was subjected to silica gel column chromatography,.and eluted with ethyl acetate-methanol (1:1). The target fraction was concentrated, and then the residue was dissolved in ethyl acetate. The mixture was acidified with a 4 N hydrogen chloride-ethyl acetate solution, and then concentrated in vacuo to give the desired product (45 mg) as an amorphous solid.

MS (ESI+, m/e) 374 (M+1)

### Example 5 (Method E)

### 2-Benzyl-1-[(1-benzyl-2-methyl-5-phenyl-1H-pyrrol-3-yl)carbonyl]piperazine hydrochloride

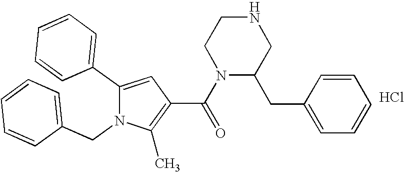

A solution of 1-benzyl-2-methyl-5-phenyl-1H-pyrrole-3-carboxylic acid (150 mg), tert-butyl 3-benzylpiperazine-1-carboxylate (140 mg), WSC.HCl (128 mg), HOBt (103 mg) and DMF (10 ml) was stirred at room temperature for 12 hr. Then, the mixture was poured into a saturated aqueous sodium bicarbonate solution and extracted with ethyl acetate. The extract was washed successively with water and brine and dried over anhydrous sodium sulfate, and then the solvent was evaporated in vacuo. The residue was subjected to silica gel column chromatography, and the fraction eluted with ethyl acetate-hexane (1:9 to 1:3) was concentrated in vacuo to give an amorphous solid (140 mg). 120 mg of the resulting amorphous was dissolved in ethyl acetate (2 ml), and a 4 N hydrogen chloride-ethyl acetate solution (2 ml) was added thereto. After stirring at room temperature for 12 hr, the reaction mixture was concentrated in vacuo to give the desired product (95 mg) as an amorphous solid.

MS (ESI+, m/e) 450 (M+1)

### Example 6 (Method F)

### Ethyl {4-[3-{[(2R)-2-benzylpiperazin-1-yl]carbonyl)-1-(2,3-dimethoxyphenyl)-5-methyl-1H-pyrrol-2-yl]phenoxy}acetate hydrochloride

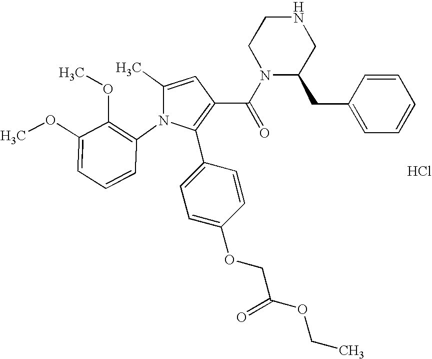

Ethyl {4-[3-{[(2R)-2,4-dibenzylpiperazin-1-yl]carbonyl}-1-(2,3-dimethoxyphenyl)-5-methyl-1H-pyrrol-2-yl]phenoxy}acetate (250 mg) was dissolved in ethanol (6 ml). Concentrated hydrochloric acid (35 μl) and 10% palladium on carbon (containing 50% water, 110 mg) were added thereto, and the mixture was subjected to catalytic hydrogenation at room temperature and atmospheric pressure for 15 hr. The catalyst was filtered off, and the filtrate was concentrated in vacuo. The residue was diluted with a saturated aqueous sodium bicarbonate solution and extracted with ethyl acetate. The extract was washed successively with water and brine and dried over anhydrous magnesium sulfate, and then the solvent was evaporated in vacuo. To the residue were added diethyl ether (3 ml) and a 4 N hydrogen chloride-ethyl acetate solution (100 μl), and the crystals were collected by filtration to give the desired product (141 mg).

MS (ESI+, m/e) 598 (M+1)

### Example 7 (Method G)

### {4-[3-{[(2R)-2-Benzylpiperazin-1-yl]carbonyl)-1-(2,3-dimethoxyphenyl)-5-methyl-1H-pyrrol-2-yl]phenoxy}acetamide hydrochloride

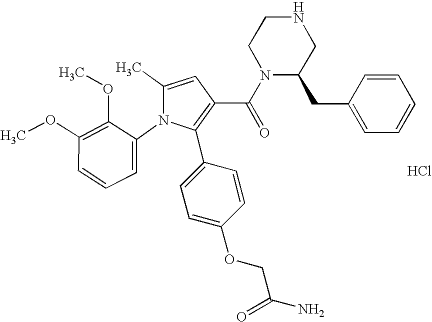

{4-[3-{[(2R)-2,4-Dibenzylpiperazin-1-yl]carbonyl}-1-(2,3-dimethoxyphenyl)-5-methyl-1H-pyrrol-2-yl]phenoxy}acetamide (348 mg) was dissolved in methanol (10 ml), 20% palladium on carbon hydroxide (containing 50% water, 180 mg) was added thereto, and the mixture was subjected to catalytic hydrogenation at room temperature and atmospheric pressure for 15 hr. The catalyst was filtered off, and the filtrate was concentrated in vacuo. To the residue were added diethyl ether (5 ml) and a 4 N hydrogen chloride-ethyl acetate solution (145 μl), and the crystals were collected by filtration to give the desired product (240 mg).

MS (ESI+, m/e) 569 (M+1)

### Example 8 (Method H)

### 4-[3-(3-{[(2R)-2-Benzylpiperazin-1-yl]carbonyl}-5-methyl-2-phenyl-1H-pyrrol-1-yl)phenyl]thiomorpholine dihydrochloride

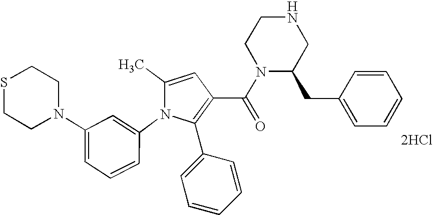

4-[3-(3-{[(2R)-2,4-Dibenzylpiperazin-1-yl]carbonyl}-5-methyl-2-phenyl-1H-pyrrol-1-yl)phenyl]thiomorpholine (100 mg) was dissolved in 1,2-dichloroethane (2 ml), and 1-chloroethyl chloroformate (52 mg) was added thereto at 0° C. The mixture was stirred at 80° C. for,2 hr, and then the solvent was evaporated in vacuo. To the residue was added methanol (3 ml), the reaction mixture was heated under reflux for 1 hr, and the solvent was then evaporated in vacuo. The residue was dissolved in ethyl acetate, washed with a saturated aqueous sodium bicarbonate solution and dried over anhydrous sodium sulfate, and then the solvent was evaporated in vacuo. The residue was subjected to silica gel column chromatography, and eluted with ethyl acetate-methanol (4:1). After concentrating the target fraction, the residue was dissolved in ethyl acetate, acidified with a 4 N hydrogen chloride-ethyl acetate solution, and then concentrated in vacuo to give the desired product (25 mg) as an amorphous solid.

MS (ESI+, m/e) 537 (M+1)

### Example 9 (Method I)

### (2R)-2-Benzyl-1-({2-[3-(benzyloxy)phenyl]-5-methyl-1-phenyl-1H-pyrrol-3-yl}carbonyl)piperazine hydrochloride

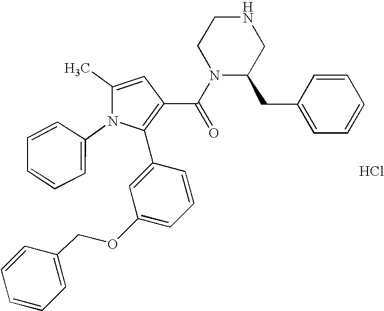

tert-Butyl (3R)-3-benzyl-4-({2-[3-(benzyloxy)phenyl]-5-methyl-1-phenyl-1H-pyrrol-3-yl)carbonyl)piperazine-1-carboxylate (321 mg) was dissolved in dichloromethane (0.4 ml), and TEA (2 ml) was added thereto. After stirring at room temperature for 30 min, the mixture was poured into a saturated aqueous sodium bicarbonate solution (100 ml) and extracted with ethyl acetate. The extract was washed successively with water and brine and dried over anhydrous magnesium sulfate, and then the solvent was evaporated in vacuo. To the residue were added diethyl ether (4 ml) and a 4 N hydrogen chloride-ethyl acetate solution (138 μl), and the crystals were collected by filtration to give the desired product (201 mg).

MS (ESI+, m/e) 542 (M+1)

### Example 10 (Method J)

### (2R)-2-Benzyl-1-{[1-(2-methoxyphenyl)-5-methyl-2-phenyl-1H-pyrrol-3-yl]carbonyl}piperazine hydrochloride

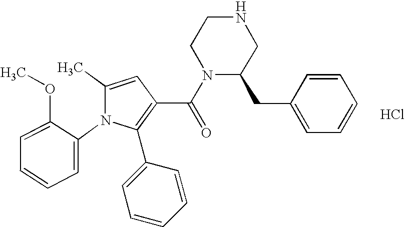

tert-Butyl (3R)-3-benzyl-4-{[1-(2-methoxyphenyl)-5-methyl-2-phenyl-1H-pyrrol-3-yl]carbonyl}piperazine-1-carboxylate (110 mg) was dissolved in ethyl acetate (2 ml), and a 4 N hydrogen chloride-ethyl acetate solution (2 ml) was added thereto. The mixture was stirred at room temperature for 12 hr, and then the solvent was evaporated in vacuo. The residue was diluted with ethyl acetate, washed with a saturated aqueous sodium bicarbonate solution and dried over anhydrous magnesium sulfate, and then the solvent was evaporated in vacuo. The residue was subjected to silica gel column chromatography and eluted with ethyl acetate-methanol (4:1). After concentrating the target fraction, the residue was dissolved in ethyl acetate, acidified with a 4 N hydrogen chloride-ethyl acetate solution, and then concentrated in vacuo to give the desired product (70 mg) as an amorphous solid.

MS (ESI+, m/e) 466 (M+1)

In the same manners as in Example 1 (Method A) to Example 10 (Method J) described above, the following compounds (Examples 11 to 158) were obtained. However, the compound indicated with “-” instead of “salt” in Tables was isolated by Method A to Method I as crystals or an amorphous solid of the free compound by omitting the treatment with a 4 N hydrogen chloride-ethyl acetate solution in the final process.

TABLE 1

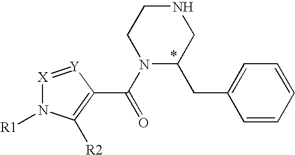

| | | | | | chiral | synthetic | | |
|---|---|---|---|---|---|---|---|---|
| Ex. No. | X | Y | R1 | R2 | center | method | salt | MS (ESI+) |
| 11 | CH | CH | Ph | Ph | S | B | HCl | 422 |
| 12 | C-Me | CH | PhCH2 | Ph | R | B | HCl | 450 |
| 13 | C-Me | CH | PhCH2 | Ph | S | B | HCl | 450 |
| 14 | C-Me | CH | 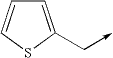 | Me | racemic | E | HCl | 394 |
| 15 | CH | CH | PhCH2 | Ph | racemic | E | HCl | 436 |
| 16 | CH | CH | 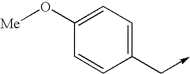 | Ph | racemic | E | HCl | 466 |
| 17 | CH | CH | 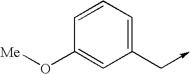 | Ph | racemic | E | HCl | 466 |
| 18 | CH | CH | 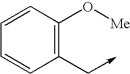 | Ph | racemic | E | HCl | 466 |
| 19 | CH | CH | 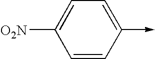 | Me | racemic | E | HCl | 405 |
| 20 | N | CH | Ph | Me | racemic | D | HCl | 361 |
| 21 | N | CH | PhCH2 | Ph | racemic | D | HCl | 437 |
| 22 | N | CH | 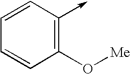 | Ph | racemic | D | HCl | 453 |
| 23 | N | CH | 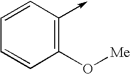 | Ph | racemic | D | HCl | 453 |
| 24 | N | CH | 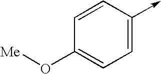 | Ph | racemic | D | HCl | 453 |
| 25 | N | CH | Ph | Ph | R | B | HCl | 423 |
| 26 | N | CH | tBu | 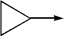 | racemic | D | HCl | 367 |
| 27 | N | CH | tBu | 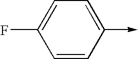 | racemic | D | HCl | 421 |
| 28 | N | CH | 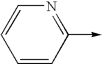 | 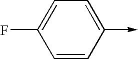 | racemic | D | HCl | 442 |
| 29 | N | CH | 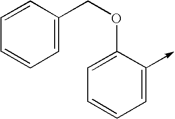 | 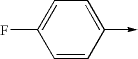 | racemic | I | HCl | 547 |
| 30 | CH | CH | 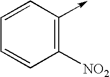 | Ph | racemic | E | HCl | 467 |
| 31 | CH | CH | 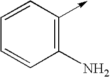 | Ph | racemic | J | 2HCl | 437 |
| 32 | CH | CH | 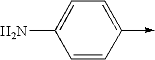 | Ph | racemic | J | 2HCl | 437 |
| 33 | N | N | Ph | Ph | racemic | D | HCl | 424 |
| 34 | N | N | Ph | 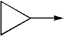 | racemic | D | HCl | 388 |
| 35 | N | N | 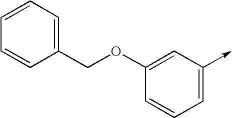 | Ph | racemic | I | HCl | 530 |
| 36 | N | N | 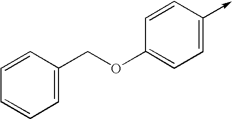 | Ph | racemic | I | HCl | 530 |
| 37 | N | N | 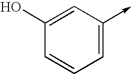 | Ph | racemic | I | HCl | 440 |
| 38 | N | N | 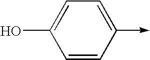 | Ph | racemic | I | HCl | 440 |
| 39 | N | N | 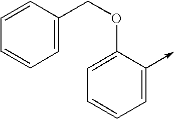 | Ph | racemic | I | HCl | 530 |
| 40 | N | N | 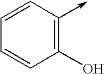 | Ph | racemic | I | HCl | 440 |
| 41 | N | N | Ph | 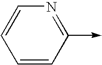 | racemic | D | 2HCl | 425 |
| 42 | CH | N | Ph | Ph | R | A | HCl | 423 |
| 43 | C-Me | CH | 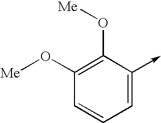 | Ph | R | B | HCl | 496 |
| 44 | N | N | 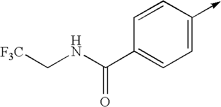 | 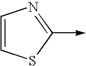 | R | D | HCl | 556 |
| 45 | N | N | 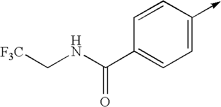 | 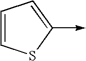 | R | D | HCl | 555 |
| 46 | N | CH | Ph | 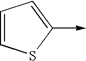 | R | D | HCl | 429 |
| 47 | C-Me | CH | 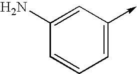 | Ph | R | I | — | 451 |
| 48 | C-Me | CH | Ph | 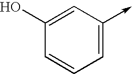 | R | I | — | 452 |
| 49 | C-Me | CH | 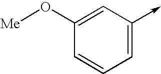 | Ph | R | B | HCl | 466 |
| 50 | C-Me | CH | 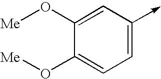 | Ph | R | B | HCl | 496 |
| 51 | C-Me | CH | 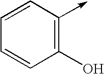 | Ph | R | G | HCl | 452 |
| 52 | N | N | 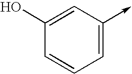 | Ph | R | I | HCl | 440 |
| 53 | C-Me | CH | Ph | 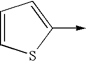 | R | D | HCl | 442 |
| 54 | C-Me | CH | 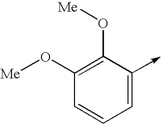 | 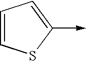 | R | D | HCl | 502 |
| 55 | C-Me | CH | 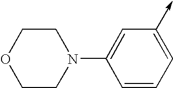 | 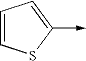 | R | D | HCl | 527 |
| 56 | CH | N | 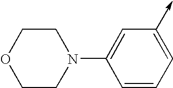 | Ph | R | B | 2HCl | 508 |
| 57 | CH | N | 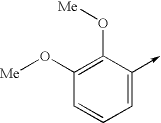 | Ph | R | A | HCl | 483 |
| 58 | C-Me | CH | 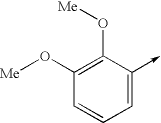 | 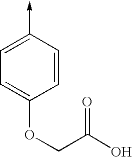 | R | G | — | 570 |
| 59 | C-Me | CH | 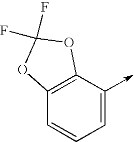 | Ph | R | B | HCl | 516 |
| 60 | C—Ac | CH | Ph | Ph | R | E | HCl | 464 |
| 61 | CH | C—Ac | Ph | Ph | R | E | HCl | 464 |
| 62 | C-Me | CH | 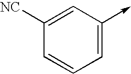 | Ph | R | H | — | 461 |
| 63 | CH | CH | 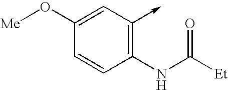 | Ph | R | I | — | 523 |
| 64 | C-Me | CH | 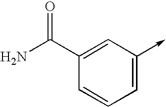 | Ph | R | G | — | 479 |
| 65 | N | CH | Ph | 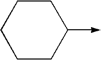 | R | D | — | 429 |
| 66 | CH | CH | 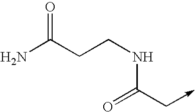 | Ph | R | G | — | 474 |
| 67 | CH | CH | 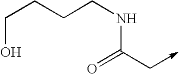 | Ph | R | G | — | 475 |
| 68 | CH | N | PhCH2 | Ph | R | D | HCl | 437 |
| 69 | CH | N | 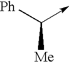 | Ph | R | D | HCl | 451 |
| 70 | CH | N | 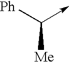 | Ph | R | D | HCl | 451 |
| 71 | CH | N | 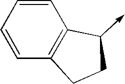 | Ph | R | D | HCl | 463 |
| 72 | CH | N | 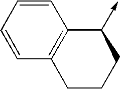 | Ph | R | D | HCl | 477 |
| 73 | CH | N | 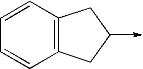 | Ph | R | D | HCl | 463 |

TABLE 2

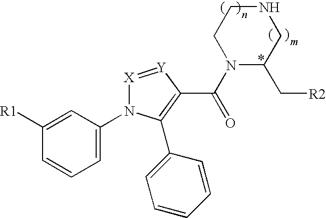

| | | | | | | | chiral | synthetic | | |
|---|---|---|---|---|---|---|---|---|---|---|
| Ex. No. | X | Y | R1 | n | m | R2 | center | method | salt | MS (ESI+) |
| 74 | 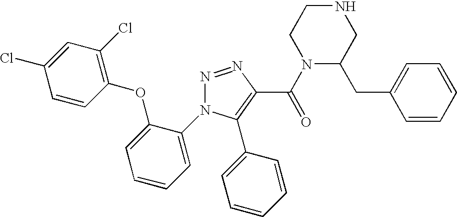 | | | | | | racemic | D | HCl | 585 |
| 75 | N | N | 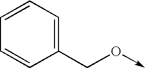 | 1 | 1 | 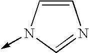 | racemic | D | HCl | 520 |
| 76 | N | N | 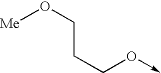 | 1 | 1 | Ph | racemic | I | HCl | 512 |
| 77 | CH | CH | H | 2 | 1 | Ph | R | A | — | 436 |
| 78 | N | CH | H | 2 | 1 | Ph | R | A | — | 437 |
| 79 | N | N | H | 2 | 1 | Ph | R | A | — | 438 |
| 80 | CH | N | H | 2 | 1 | Ph | R | A | HCl | 437 |
| 81 | N | CH | H | 1 | 1 | 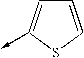 | R | C | — | 429 |
| 82 | N | N | H | 1 | 1 | 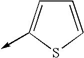 | R | C | — | 430 |
| 83 | CH | CH | H | 1 | 2 | Ph | S | B | — | 436 |
| 84 | N | CH | H | 1 | 2 | Ph | S | B | — | 437 |
| 85 | N | N | H | 1 | 2 | Ph | S | B | — | 438 |
| 86 | N | N | 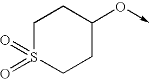 | 1 | 1 | Ph | R | I | — | 572 |
| 87 | N | N | 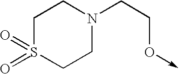 | l | 1 | Ph | R | I | — | 601 |
| 88 | C-Me | CH | H | 1 | 1 | OH | racemic | D | HCl | 376 |
| 89 | C-Me | CH | H | 1 | 1 | H | S | I | — | 376 |

(Note: The binding position of substituent R¹ is the 2 position for Example 74 alone, and the 3-position for others.)

TABLE 3

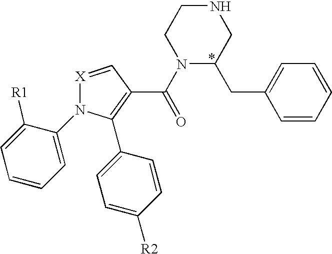

| | | | | chiral | synthetic | | |
|---|---|---|---|---|---|---|---|
| Ex. No. | X | R1 | R2 | center | method | salt | MS (ESI+) |
| 90 | CH | 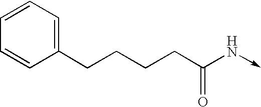 | H | racemic | J | HCl | 597 |
| 91 | N | 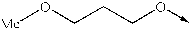 | F | racemic | I | HCl | 529 |
| 92 | N | 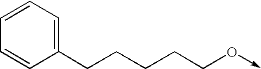 | F | racemic | I | HCl | 603 |
| 93 | N | 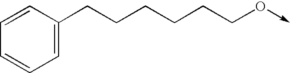 | F | racemic | I | HCl | 617 |
| 94 | CH | 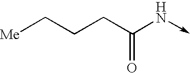 | H | racemic | J | HCl | 521 |
| 95 | CH | 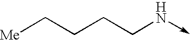 | H | racemic | J | 2HCl | 507 |
| 96 | CH | 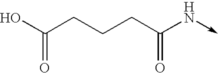 | H | racemic | J | HCl | 551 |
| 97 | CH | 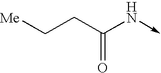 | H | racemic | J | HCl | 507 |
| 98 | CH | 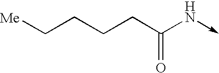 | H | racemic | J | HCl | 535 |
| 99 | CH | 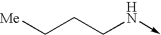 | H | racemic | I | 2HCl | 493 |
| 100 | CH | 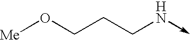 | H | racemic | J | 2HCl | 509 |
| 101 | CH | 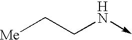 | H | racemic | I | 2HCl | 479 |
| 102 | CH | 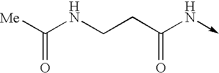 | H | R | I | HCl | 550 |

TABLE 4

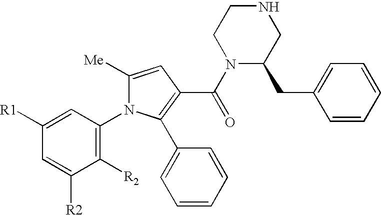

| | | | synthetic | | |
|---|---|---|---|---|---|
| Ex. No. | R1 | R2 | method | salt | MS (ESI+) |
| 103 | 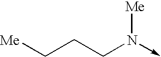 | H | G | 2HCl | 521 |
| 104 | 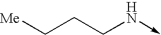 | H | G | 2HCl | 507 |
| 105 | 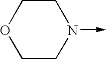 | H | G | 2HCl | 521 |
| 106 | 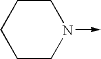 | H | G | 2HCl | 519 |
| 107 | 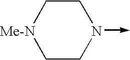 | H | G | 3HCl | 534 |
| 108 | 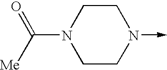 | H | G | — | 562 |
| 109 | 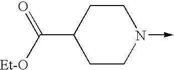 | H | G | 2HCl | 591 |
| 110 | 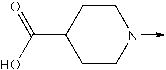 | H | G | 2HCl | 563 |
| 111 | 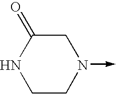 | H | G | — | 534 |
| 112 | 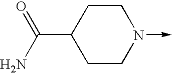 | H | G | 2HCl | 562 |
| 113 | 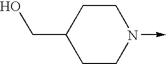 | H | G | 2HCl | 549 |
| 114 | 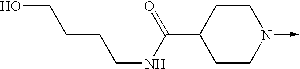 | H | G | 2HCl | 634 |
| 115 | 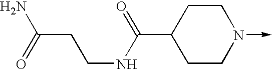 | H | G | — | 633 |
| 116 | 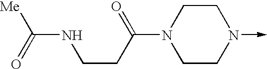 | H | G | — | 633 |
| 117 | 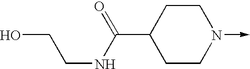 | H | G | — | 606 |
| 118 | 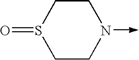 | H | I | — | 553 |
| 119 | 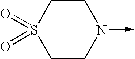 | H | I | — | 569 |
| 120 | 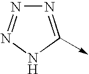 | H | G | — | 504 |
| 121 | 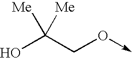 | H | I | — | 524 |
| 122 | 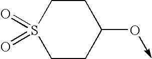 | H | I | — | 584 |
| 123 | 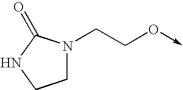 | H | I | — | 564 |
| 124 | 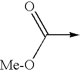 | OMe | G | HCl | 554 |
| 125 | 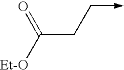 | OMe | G | HCl | 596 |
| 126 | 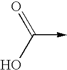 | OMe | G | — | 540 |
| 127 | 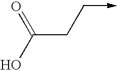 | OMe | G | — | 568 |
| 128 | 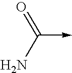 | OMe | G | HCl | 539 |
| 129 | 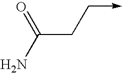 | OMe | G | HCl | 567 |
| 130 | 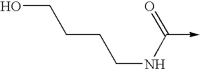 | OMe | G | — | 611 |
| 131 | 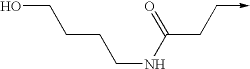 | OMe | G | HCl | 639 |
| 132 | 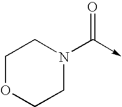 | OMe | G | HCl | 609 |
| 133 | 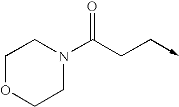 | OMe | G | HCl | 637 |

TABLE 5

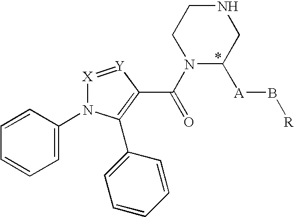

| | | | | | | chiral | synthetic | | |
|---|---|---|---|---|---|---|---|---|---|
| Ex. No. | X | Y | A | B | R | center | method | salt | MS(ESI+) |
| 134 | CH | CH | CH2 | CH2 | Ph | S | A | HCl | 436 |
| 135 | N | CH | CH2 | CH2 | Ph | R | A | — | 437 |
| 136 | N | N | CH2 | CH2 | Ph | R | A | — | 438 |
| 137 | CH | N | CH2 | CH2 | Ph | R | A | — | 437 |
| 138 | C-Me | CH | bond | bond | iPr | R | B | — | 388 |
| 139 | N | CH | CH2 | O | Ph | racemic | D | HCl | 439 |
| 140 | N | N | CH2 | O | Ph | racemic | D | HCl | 440 |
| 141 | CH | CH | CH2 | O | Ph | racemic | D | HCl | 438 |
| 142 | N | N | CH2 | O | 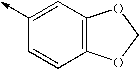 | racemic | D | HCl | 484 |
| 143 | C-Me | CH | CH2 | NH | 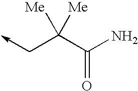 | racemic | D | — | 474 |
| 144 | C-Me | CH | C═O | NH | 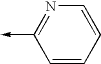 | racemic | I | — | 466 |
| 145 | C-Me | CH | C═O | NH | CH2Ph | racemic | I | — | 479 |

TABLE 6

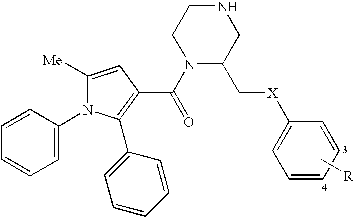

| | | subsutituted | | synthetic | | |
|---|---|---|---|---|---|---|
| Ex. No. | X | position | R | method | salt | MS(ESI+) |
| 146 | bond | 4- | 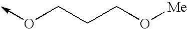 | B | HCl | 524 |
| 147 | bond | 4- | 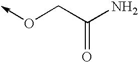 | G | — | 509 |
| 148 | bond | 4- | 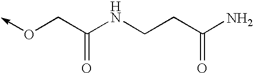 | G | HCl | 580 |
| 149 | bond | 4- | 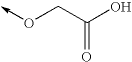 | G | — | 510 |
| 150 | bond | 4- | OH | G | — | 452 |
| 151 | bond | 4- | 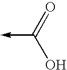 | G | — | 480 |
| 152 | bond | 4- | 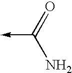 | G | — | 479 |
| 153 | bond | 4- | 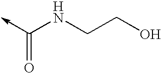 | G | — | 523 |
| 154 | O | 4- | 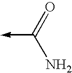 | I | — | 495 |
| 155 | O | 4- | 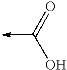 | I | — | 496 |
| 156 | O | 4- | 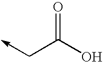 | I | — | 510 |
| 157 | O | 4- | 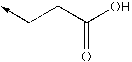 | I | — | 524 |
| 158 | O | 3- | 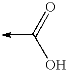 | I | — | 496 |

### Example 56

### 4-[3-(4-{[(2R)-2-Benzylpiperazin-1-yl]carbonyl}-5-phenyl-1H-imidazol-1-yl)phenyl]morpholine dihydrochloride

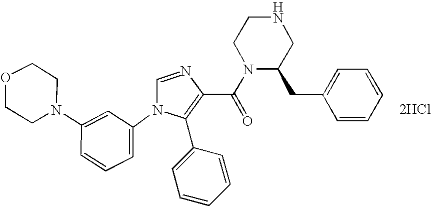

A solution of 1-(3-morpholinophenyl)-5-phenyl-1H-imidazole-4-carboxylic acid (262 mg), (3R)-1,3-dibenzylpiperazine (200 mg), WSC.HCl (173 mg) and HOBt (122 mg), DMF (5 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane (1:2 to 1:1) was concentrated in vacuo to give an amorphous solid (370 mg). The total amount thereof was dissolved in methanol (10 ml), 20% palladium hydroxide on carbon (containing 50% water) (180 mg) was added, and a catalytic hydrogenation was performed at room temperature and atmospheric pressure for 15 hr. The catalyst was filtered off, and the filtrate was concentrated in vacuo. The residue was diluted with diethyl ether (8 ml), a 4 N hydrogen chloride-ethyl acetate solution (340 μl) was added, and the precipitated crystals were collected by filtration to give the desired product (242 mg).

MS (ESI+, m/e) 508 (M+1)

### Example 57

### (2R)-2-Benzyl-1-{[1-(2,3-dimethoxyphenyl)-5-phenyl-1H-imidazol-4-yl]carbonyl}piperazine hydrochloride

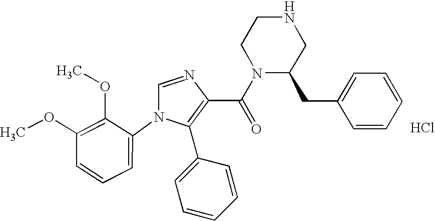

A solution of 1-(2,3-dimethoxyphenyl)-5-phenyl-1H-imidazole-4-carboxylic acid (119 mg), (3R)-1,3-dibenzylpiperazine (98 mg), WSC.HCl (84 mg), HOBt (59 mg) and DMF (2.5 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-hexane (1:2 to 1:1) was concentrated in vacuo to give an amorphous solid (170 mg). The total amount thereof was dissolved in methanol (5 ml), 20% palladium hydroxide on carbon (containing 50% water) (85 mg) was added, and a catalytic hydrogenation was performed at room temperature and atmospheric pressure for 15 hr. The catalyst was filtered off, and the filtrate was concentrated in vacuo. The residue was diluted with diethyl ether (2 ml), and a 4 N hydrogen chloride-ethyl acetate solution (84 μl) was added, and the precipitated crystals were collected by filtration to give the desired product (92 mg).

MS (ESI+, m/e) 483 (M+1)

### Example 73

### (2R)-2-Benzyl-1-{[1-(2,3-dihydro-1H-inden-2-yl)-5-phenyl-1H-imidazol-4-yl]carbonyl}piperazine hydrochloride

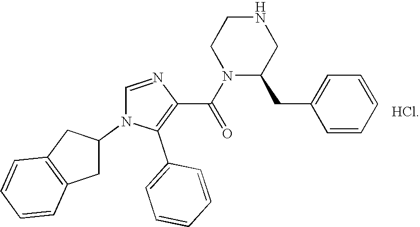

A solution of 1-(2,3-dihydro-1H-inden-2-yl)-5-phenyl-1H-imidazole-4-carboxylic acid (228 mg), tert-butyl 3-benzylpiperazine-1-carboxylate (218 mg), WSC.HCl (173 mg), HOBt (122 mg) and DMF (5 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, to and dried over anhydrous magnesium sulfate. The solvent was evaporated in vacuo. The residue was dissolved in dichloromethane (0.5 ml), TFA (1.5 ml) was added, and the mixture was stirred at room temperature for 30 min. The reaction mixture was poured into saturated aqueous sodium bicarbonate solution by small portions. The mixture was saturated with sodium chloride, and extracted with ethyl acetate. The extract was washed with brine, and dried over anhydrous magnesium sulfate. The solvent was evaporated in vacuo. The residue was subjected to basic silica gel column chromatography, and the fraction eluted with ethyl acetate-methanol (1:0 to 20:1) was concentrated in vacuo. The residue was diluted with diethyl ether (6 ml), a 4 N hydrogen chloride-ethyl acetate solution (188 μl) was added, and the precipitated crystals were collected by filtration to give the desired product (245 mg).

MS (ESI+, m/e) 463 (M+1)

In the same manner as in Example 2 (Method B), the following compound (Example 159) was obtained.

### Example 159

### (2R)-2-Benzyl-1-{[1-(3-methoxypropyl)-4,5-diphenyl-1H-pyrrol-3-yl]carbonyl}piperazine hydrochloride

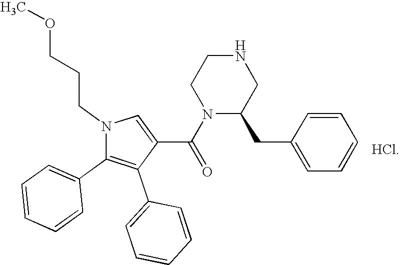

MS (ESI+, m/e) 494 (M+1)

In the same manner as in Example 4 (Method D), the following compound (Example 160) was obtained. However, the final product was isolated as an amorphous solid of the free compound by omitting the treatment with a 4 N hydrogen chloride-ethyl acetate solution.

### Example 160

### (2R)-2-Benzyl-1-[(3,4-diphenyl-2-thienyl)carbonyl]piperazine

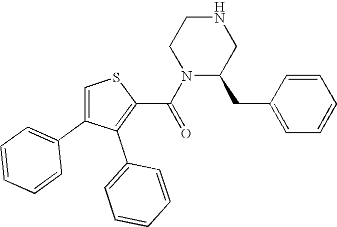

MS (ESI+, m/e) 439 (144-1)

In the same manner as in Example 7 (Method G), the following compound (Example 161) was obtained. However, the final product was isolated as an amorphous solid of the free compound by omitting the treatment with a 4 N hydrogen chloride-ethyl acetate solution.

### Example 161

### 4-[((2R)-1-{[1-(3-Morpholinophenyl)-5-phenyl-1H-imidazol-4-yl]carbonyl}piperazin-2-yl)methyl]benzoic acid

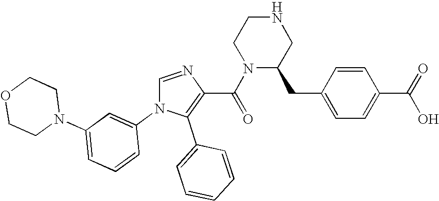

MS (ESI+, m/e) 552 (M+1)

In the same manner as in Example 9 (Method I), the following compound (Example 162) was obtained.

### Example 162

### N-(4-{3-[(2-Benzylpiperazin-1-yl)carbonyl]-2-methyl-1H-pyrrol-1-yl}phenyl)-5-phenylpentanamide hydrochloride

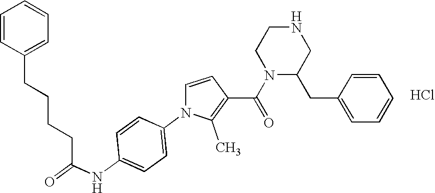

MS (ESI+, m/e) 535 (M+1)

In the same manner as in Example 10 (Method J), the following compound (Example 163) was obtained.

### Example 163

### N-(4-{3-[(2-Benzylpiperazin-1-yl)carbonyl]-2-phenyl-1H-pyrrol-1-yl}phenyl)-5-phenylpentahamide hydrochloride

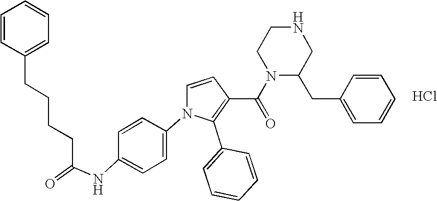

MS (ESI+, m/e) 597 (M+1)

### Example 164

### (2R)-2-Benzyl-1-[(5-methyl-1-{3-[3-(methylsulfonyl)propoxy]phenyl}-2-phenyl-1H-pyrrol-3-yl)carbonyl]piperazine

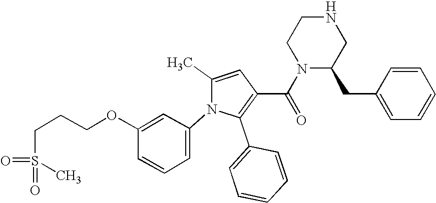

To a solution of tert-butyl(3R)-3-benzyl-4-[(5-methyl-1-{3-[3-(methylsulfonyl)propoxy]phenyl}-2-phenyl-1H-pyrrol-3-yl)carbonyl]piperazine-1-carboxylate (180 mg) in ethyl acetate (3 ml) was added a 4 N hydrogen chloride-ethyl acetate solution. The mixture was stirred at room temperature for 12 hr, and then the solvent was evaporated in vacuo. The residue was suspended in ethyl acetate, and the reaction suspension was washed with a saturated aqueous sodium bicarbonate solution and dried over anhydrous magnesium sulfate, and then the solvent was evaporated in vacuo. The residue was subjected to silica gel column chromatography, and the fraction eluted with ethyl acetate-methanol (4:1) was concentrated in vacuo to give the desired product (130 mg) as an amorphous solid.

MS (ESI+, m/e) 572 (M+1)

### Example 165

### 1-[2-(3-{[(2R)-2-Benzylpiperazin-1-yl]carbonyl}-5-methyl-2-phenyl-1H-pyrrol-1-yl)phenyl]methylamine dihydrochloride

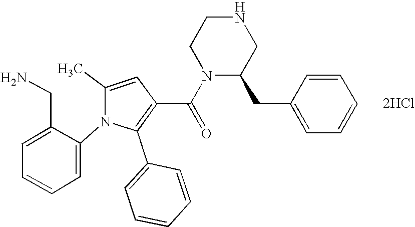

tert-Butyl(2R)-(2-{3-[(2,4-dibenzylpiperazin-1-yl)carbonyl]-5-methyl-2-phenyl-1H-pyrrol-1-yl}benzyl)carbamate (160 mg) was dissolved in methanol (15 ml), 20% palladium on carbon hydroxide (containing 50% water, 50 mg) was added thereto, and the mixture was subjected to catalytic hydrogenation at room temperature and atmospheric pressure for 12 hr. The catalyst was filtered off, and the filtrate was concentrated in vacuo. The residue was subjected to silica gel column chromatography, and the fraction eluted with ethyl acetate-methanol (1:0 to 4:1) was concentrated in vacuo to give an amorphous solid (90 mg). 80 mg of the resulting amorphous was dissolved in dichloromethane (5 ml), and TFA (3 ml) was added thereto. The mixture was stirred at room temperature for 2 hr, and then the solvent was evaporated in vacuo. The residue was subjected to reverse-phase HPLC analysis (purification condition is described above), and the target fraction was neutralized with a saturated aqueous sodium bicarbonate solution and extracted with ethyl acetate. The extract was dried over anhydrous sodium sulfate, and the solvent was evaporated. The residue was dissolved in ethyl acetate, acidified with a 4 N hydrogen chloride-ethyl acetate solution, and then concentrated in vacuo to give the desired product (56 mg) as an amorphous solid.

MS (ESI+, m/e) 465 (M+1)

### Example 166

### (2R)-2-Benzyl-1-[(5-methyl-1,2-diphenyl-1H-pyrrol-3-yl)carbonyl]piperazine hydrochloride

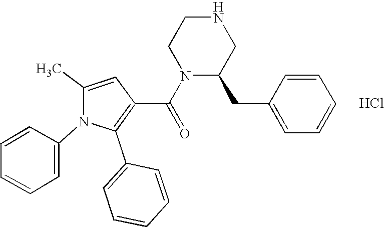

(2R)-2,4-Dibenzyl-1-{[1-(3-bromophenyl)-5-methyl-2-phenyl-1H-pyrrol-3-yl]carbonyl}piperazine (400 mg) was dissolved in methanol (10 ml), 20% palladium on carbon hydroxide (containing 50% water, 100 mg) was added thereto, and the mixture was subjected to catalytic hydrogenation at room temperature and atmospheric pressure for 12 hr. The catalyst was filtered off, and the filtrate was concentrated in vacuo. The residue was diluted with ethyl acetate, and the reaction mixture was washed with a saturated aqueous sodium bicarbonate solution and dried over anhydrous sodium sulfate, and then the solvent was evaporated in vacuo. The residue was subjected to silica gel column chromatography and eluted with ethyl acetate-methanol (4:1). The target fraction was concentrated, and then the residue was dissolved in ethyl acetate. The mixture was acidified with a 4 N hydrogen chloride-ethyl acetate solution, and then concentrated in vacuo to give the desired product (310 mg) as an amorphous solid.

MS (ESI+, m/e) 436 (M+1)

In the same manner as in Example 166, the following compound (Example 167) was obtained.

### Example 167

### 2-(2-Methoxybenzyl)-1-[(5-methyl-1,2-diphenyl-1H-pyrrol-3-yl)carbonyl]piperazine hydrochloride

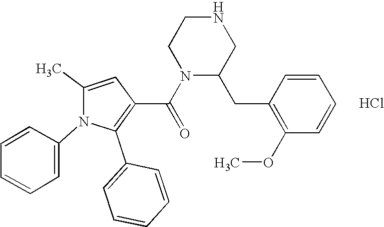

MS (ESI+, m/e) 466 (M+1)

### Example 168

### [3-(3-{[(2R)-2-Benzylpiperazin-1-yl]carbonyl}-5-methyl-1-phenyl-1H-pyrrol-2-yl)phenoxy]acetic acid

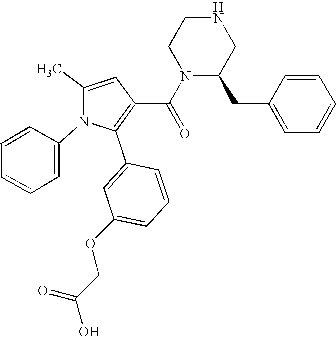

A suspension of tert-butyl(3R)-3-benzyl-4-{[2-(3-hydroxyphenyl)-5-methyl-1-phenyl-1H-pyrrol-3-yl]carbonyl}piperazine-1-carboxylate (300 mg), tert-butyl bromoacetate (117 mg), potassium carbonate (90 mg) and DMF (2 ml) was stirred at 70° C. for 3 hr. Then, the mixture was poured into water and extracted with ethyl acetate. The extract was washed successively with water and brine and dried over anhydrous magnesium sulfate, and then the solvent was evaporated in vacuo. The residue was subjected to silica gel column chromatography, and the fraction eluted with ethyl acetate-hexane (1:2) was concentrated in vacuo. To the resulting oil, was added a 4 N hydrogen chloride-ethyl acetate solution (3 ml), and the mixture was stirred at room temperature for 3 hr. The reaction mixture was concentrated in vacuo, and the residue was dissolved in a 1% aqueous potassium carbonate solution (50 ml) and washed with ethyl acetate. The aqueous layer was neutralized with 2 N hydrochloric acid, then saturated with sodium chloride and extracted with ethyl acetate-THF (3:1). The extract was washed with brine and dried over anhydrous magnesium sulfate. Then, the solvent was evaporated in vacuo, and the crystals were collected by filtration to give the desired product (105 mg).

MS (ESI+, m/e) 510 (M+1)

### Example 169

### (2R)-2-Isobutyl-1-[(5-methyl-1,2-diphenyl-1H-pyrrol-3-yl)carbonyl]piperazine hydrochloride

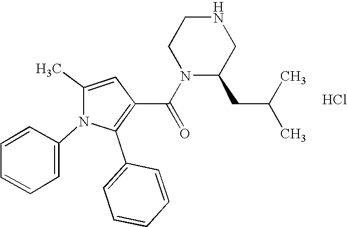

To a solution of 5-methyl-1,2-diphenyl-1H-pyrrole-3-carboxylic acid (232 mg) in dichloromethane (7 ml), was added DMF (40 mg). With ice cooling, oxalyl chloride (254 mg) was added thereto. The mixture was stirred at room temperature for 30 min and at 50° C. for 1 hr, and then the solvent was evaporated in vacuo. The residue was dissolved in dichloromethane (3 ml), and the mixture was added to a solution of ice-cooled (3R)-1-benzyl-3-isobutylpiperazine (180 mg), triethylamine (93 mg) and dichloromethane (7 ml). After stirring at room temperature for 1 hr, the mixture was poured into a saturated aqueous sodium bicarbonate solution and extracted with chloroform. The extract was dried over anhydrous sodium sulfate, and then the solvent was evaporated in vacuo. The residue was subjected to silica gel column chromatography, and the fraction eluted with ethyl acetate-hexane (1:1) was concentrated in vacuo to give an amorphous solid (150 mg). 130 mg of the resulting amorphous was dissolved in methanol (5 ml), 20% palladium on carbon hydroxide (containing 50% water, 30 mg) was added thereto, and the mixture was subjected to catalytic hydrogenation at room temperature and atmospheric pressure for 12 hr. The catalyst was filtered off, and the filtrate was concentrated in vacuo. The residue was subjected to silica gel column chromatography and eluted with ethyl acetate-methanol (1:0 to 4:1). The target fraction was concentrated, and then the residue was dissolved in ethyl acetate. The reaction mixture was acidified with a 4 N hydrogen chloride-ethyl acetate solution, and then concentrated in vacuo to give the desired product (70 mg) as an amorphous solid.

MS (ESI+, m/e) 402 (M+1)

In the same manner as in Example 169, the following compounds (Examples 170 to 171) were obtained. However, the final product was isolated as an amorphous solid of the free compound without treatment by a 4 N hydrogen chloride-ethyl acetate solution.

### Example 170

### 4-[3-(4-{[(2R)-2-Isobutylpiperazin-1-yl]carbonyl}-5-phenyl-1H-imidazol-1-yl)phenyl]morpholine

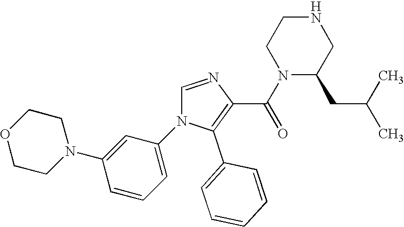

MS (ESI+, m/e) 474 (M+1)

### Example 171

### (2R)-1-{[1-(2,3-Dimethoxyphenyl)-5-methyl-2-phenyl-1H-pyrrol-3-yl]carbonyl}-2-isobutylpiperazine

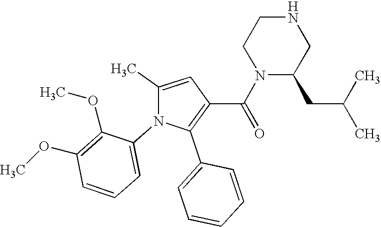

MS (ESI+, m/e) 462 (M+1)

### Example 172

### N-({(2R)-1-[(5-Methyl-1,2-diphenyl-1H-pyrrol-3-yl)carbonyl]piperazin-2-yl}methyl)aniline dihydrochloride

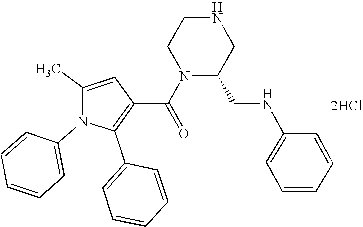

A solution of (2R)-4-benzyl-1-[(5-methyl-1,2-diphenyl-1H-pyrrol-3-yl)carbonyl]piperazine-2-carbaldehyde (250 mg), aniline (100 mg), acetic acid (65 mg), dichloromethane (2 ml) and DMF (1 ml) was stirred at room temperature for 40 min. Then, sodium triacetoxyborohydride (229 mg) was added thereto, and further stirred at room temperature for 18 hr. The reaction mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to basic silica gel column chromatography, and the fraction eluted with ethyl acetate-hexane (1:2 to 1:1) was concentrated in vacuo. The resulting amorphous (256 mg) was dissolved in acetic acid (20 ml), 20% palladium on carbon hydroxide (containing 50% water, 260 mg) was added thereto, and the mixture was subjected to catalytic hydrogenation at room temperature for 5 hr by pressurizing to 5 kgf/cm2. The catalyst was filtered off, and the filtrate was concentrated in vacuo. The residue was diluted with ethyl acetate-THF (3:1), and the reaction mixture was washed successively with a saturated aqueous sodium bicarbonate solution and brine and dried over anhydrous magnesium sulfate, and then the solvent was evaporated in vacuo. The residue was subjected to reverse-phase HPLC analysis (the purification condition is described above). The target fractions were collected, diluted with a saturated aqueous sodium bicarbonate solution and extracted with ethyl acetate. The extract was washed with brine and dried over anhydrous magnesium sulfate, and then the solvent was evaporated in vacuo. To the residue (10 mg) were added diethyl ether (1 ml) and a 4 N hydrogen chloride-ethyl acetate solution (13 μl), and the crystals were collected by filtration to give the desired product (7 mg).

MS (ESI+, m/e) 451 (M+1)

In the same manner as in Example 172, the following compound (Example 173) was obtained.

### Example 173

### N-({2S)-1-[(5-Methyl-1,2-diphenyl-1H-pyrrol-3-yl)carbonyl]piperazin-2-yl}methyl)aniline dihydrochloride

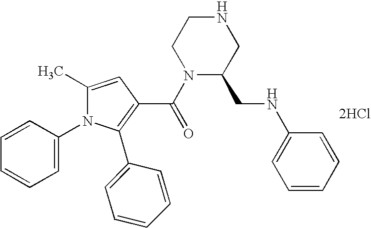

MS (ESI+, m/e) 451 (M+1)

### Example 174

N-Benzyl-1-{(2R)-1-[(5-methyl-1,2-diphenyl-1H-pyrrol-3-yl)carbonyl]piperazin-2-yl}methylamine dihydrochloride

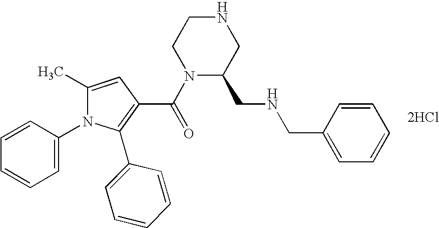

A solution of tert-butyl(3S)-3-formyl-4-[(5-methyl-1,2-diphenyl-1H-pyrrol-3-yl)carbonyl]piperazine-1-carboxylate (200 mg), benzylamine (91 mg), acetic acid (51 mg), dichloromethane (1.6 ml) and DMF (0.8 ml) was stirred at room temperature for 40 min. Sodium triacetoxyborohydride (179 mg) was added thereto, and further stirred at room temperature for 15 hr. The reaction mixture was poured into a saturated aqueous sodium bicarbonate solution and extracted with ethyl acetate. The extract was washed successively with water and brine and dried over anhydrous magnesium sulfate, and then the solvent was evaporated in vacuo. The residue was subjected to silica gel column chromatography, and the fraction eluted with ethyl acetate-hexane (1:1 to 1:0) was concentrated in vacuo. The residue was dissolved in dichloromethane (0.5 ml), and TFA (1.5 ml) was added thereto. After stirring at room temperature for 30 min, the mixture was poured into a saturated aqueous sodium bicarbonate solution (100 ml) and extracted with ethyl acetate. The extract was washed successively with water and brine and dried over anhydrous magnesium sulfate, and then the solvent was evaporated in vacuo. To the residue were added diethyl ether (5 ml) and a 4 N hydrogen chloride-ethyl acetate solution (211 μl), and the crystals were collected by filtration to give the desired product (66 mg).

MS (ESI+, m/e) 465 (M+1)

### Example 175

### (2R)-2-Benzyl-1-[(1,5-diphenyl-1H-1,2,3-triazol-4-yl)methyl]piperazine

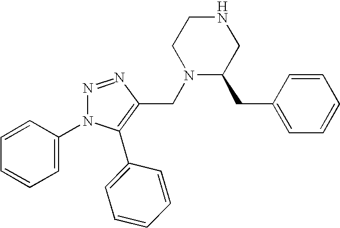

To a solution of 1,5-diphenyl-1H-1,2,3-triazole-4-carbaldehyde (249 mg) and tert-butyl (3R)-3-benzylpiperazine-1-carboxylate (276 mg) in dichloroethane (5 ml) were added acetic acid (60 mg) and sodium triacetoxyborohydride (276 mg). The mixture was stirred at room temperature for 15 hr, and then neutralized with a 6% aqueous sodium bicarbonate solution. The organic layer was washed with brine and dried over anhydrous magnesium sulfate, and then the solvent was evaporated in vacuo. The residue was subjected to basic silica gel column chromatography, and the fraction eluted with ethyl acetate-hexane (1:1) was concentrated in vacuo to give the desired product (305 mg) as an amorphous solid. The resulting product was dissolved in dichloromethane (0.5 ml), and TFA (0.5 ml) was added thereto. After stirring at room temperature for 1 hr, the mixture was concentrated in vacuo and neutralized by adding a 6% aqueous sodium bicarbonate solution to the residue. The liberated oil was extracted with chloroform, and the extract was washed with brine and dried over anhydrous magnesium sulfate. Then, the solvent was evaporated in vacuo to give the desired product (205 mg) as an amorphous solid.

MS (ESI+, m/e) 410 (M+1)

### Example 176

### 2-Benzyl-1-[(1,2-diphenyl-1H-pyrrol-3-yl)carbonyl]piperazine trifluoroacetate

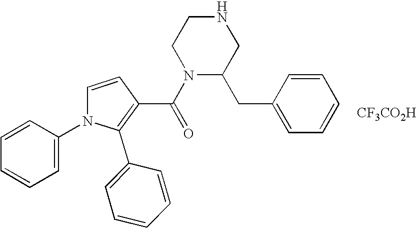

To 1,2-diphenyl-1H-pyrrole-3-carboxylic acid (a 0.15 M mixed solution of DMF-dichloromethane (1:1), 1.0 ml), was added a solution of tert-butyl3-benzylpiperazine-1-carboxylate (0.10 M), triethylamine (0.15 M), WSC.HCl (0.16 M) and HOBt (0.15 M) in dichloromethane (1.0 ml), and the mixture was agitated at room temperature for 17 hr. To the reaction mixture, were added dichloromethane (3 ml) and a 5% aqueous sodium bicarbonate solution (2 ml), agitated, and the upper layer was removed. Then, water (2 ml) was added thereto, agitated and separated. The aqueous layer was extracted with dichloromethane (1 ml), and the organic layers were combined and concentrated in vacuo with a Genevac's centrifugal concentrator. To the residue, was added TFA (a 50% dichloromethane solution, 3 ml), and the mixture was left to stand at room temperature for 2.5 hr, and then concentrated in vacuo with a Genevac's centrifugal concentrator. The residue was subjected to reverse-phase HPLC analysis (the purification condition is described above), and the target fraction was concentrated in vacuo to give the desired product (39.6 mg).

MS (ESI+, m/e) 422 (M+1)

In the same manner as in Example 176, the following compounds (Examples 177 to 182) were obtained using tert-butyl 3-benzylpiperazine-1-carboxylate.

TABLE 7

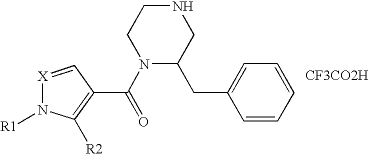

| Ex. No. | X | R1 | R2 | MS(ESI+) |
|---|---|---|---|---|
| 177 | N | Me | Ph | 361 |
| 178 | N | Ph | nPr | 389 |
| 179 | N | Ph | Ph | 423 |
| 180 | N | 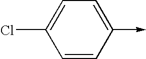 | nPr | 423 |
| 181 | C-Me | PhCH2 | Ph | 450 |
| 182 | N | tBu | 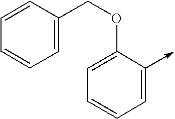 | 509 |

### Example 183

### 2-(3-{[(2R)-2-Benzylpiperazin-1-yl]carbonyl}-2-phenyl-1H-pyrrol-1-yl)-N-(2,2-dimethylpropyl)aniline

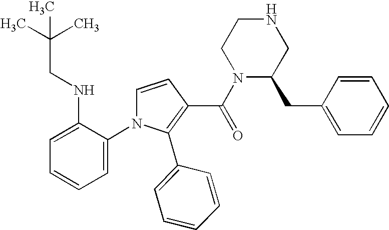

tert-Butyl(3R)-4-{(1-(2-aminophenyl)-2-phenyl-1H-pyrrol-3-yl]carbonyl}-3-benzylpiperazine-1-carboxylate (a 0.2 M solution in ethanol, 500 μl) and pivalic aldehyde (a 0.6 M solution in ethanol, 1000 μl) were mixed and stirred at 80° C. for 16 hr. Sodium triacetoxyborohydride (170 mg) was added thereto at room temperature and further stirred for 3 hr. While the reaction mixture was stirred, a 5% aqueous sodium bicarbonate solution (1 ml) was added thereto and stirred for 3 hr. The reaction mixture was heated and concentrated by a nitrogen spraying apparatus. To the residue, were added water (2 ml) and dichloromethane (2 ml), and the mixture was extracted and fractionated on a PTFE tube (a polytetrafluoroethylene film processed tube, manufactured by Whatman). To the aqueous layer, was further added dichloromethane (2 ml) and extracted. The dichloromethane layers were combined and further concentrated by a nitrogen spraying apparatus. TFA (a 10% solution in dichloromethane, 3 ml) was added thereto, and the final solution was left to stand and concentrated by a nitrogen spraying apparatus. The residue was purified with reverse-phase HPLC analysis. The target fraction was heated and concentrated by a nitrogen spraying apparatus through a MP—CO₃H resin (manufactured by Polymer Laboratories Ltd.) to give the desired product (1.2 mg).

MS (ESI+) 507 (M+1)

In the same manner as in Example 183, the following compounds (Examples 184 to 223) were obtained.

TABLE 8

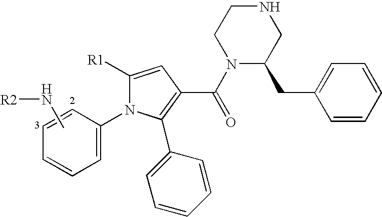

| | | substi- | | |
|---|---|---|---|---|
| | | tuted | | |
| Ex. No. | R1 | position | R2 | MS(ESI+) |
| 184 | H | 2- | 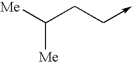 | 507 |
| 185 | H | 2- | nBu | 493 |
| 186 | H | 2- | 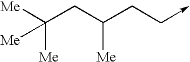 | 563 |
| 187 | H | 2- | 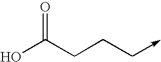 | 523 |
| 188 | H | 2- | 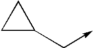 | 491 |
| 189 | H | 2- | 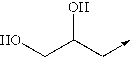 | 511 |
| 190 | H | 2- | 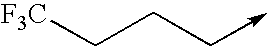 | 547 |
| 191 | H | 2- | 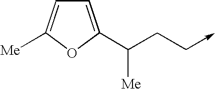 | 573 |
| 192 | H | 2- | 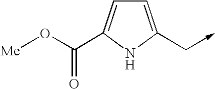 | 574 |
| 193 | Me | 3- | 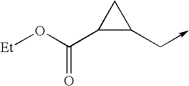 | 577 |
| 194 | Me | 3- | 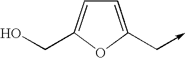 | 561 |
| 195 | Me | 3- | PhCH2 | 541 |
| 196 | Me | 3- | 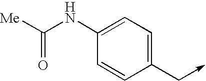 | 598 |
| 197 | Me | 3- | 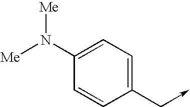 | 584 |
| 198 | Me | 3- | 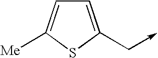 | 561 |
| 199 | Me | 3- | 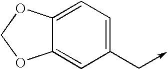 | 585 |
| 200 | Me | 3- | 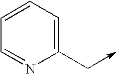 | 542 |
| 201 | Me | 3- | 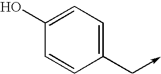 | 557 |
| 202 | Me | 3- | 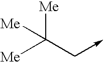 | 521 |
| 203 | Me | 3- | 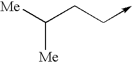 | 521 |
| 204 | Me | 3- | 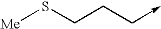 | 539 |
| 205 | Me | 3- | nBu | 507 |
| 206 | Me | 3- | 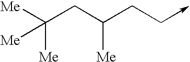 | 577 |
| 207 | Me | 3- | 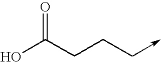 | 537 |
| 208 | Me | 3- | 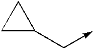 | 505 |
| 209 | Me | 3- | 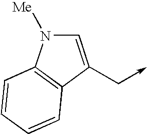 | 594 |
| 210 | Me | 3- | 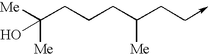 | 607 |
| 211 | Me | 3- | 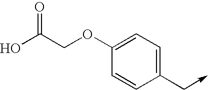 | 615 |
| 212 | Me | 3- | 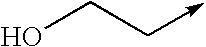 | 495 |
| 213 | Me | 3- | 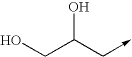 | 525 |
| 214 | Me | 3- | 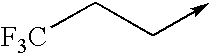 | 547 |
| 215 | Me | 3- | 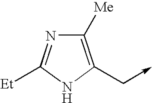 | 573 |
| 216 | Me | 3- | 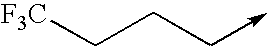 | 561 |
| 217 | Me | 3- | 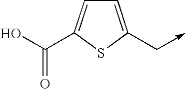 | 591 |
| 218 | Me | 3- | 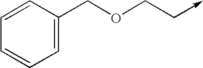 | 585 |
| 219 | Me | 3- | 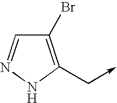 | 609 |
| 220 | Me | 3- | 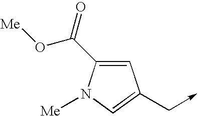 | 602 |
| 221 | Me | 3- | 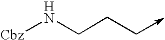 | 642 |
| 222 | Me | 3- | 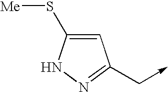 | 577 |
| 223 | Me | 3- | 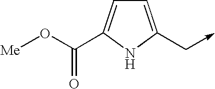 | 588 |

### Example 224

### (2R)-1-{[2-(3-{[(2R)-2-Benzylpiperazin-1-yl]carbonyl}-2-phenyl-1H-pyrrol-1-yl)phenyl]amino}propan-2-ol

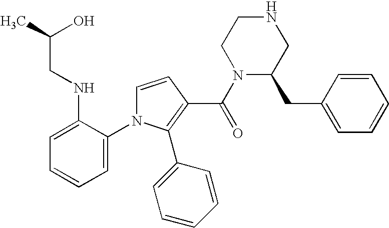

tert-Butyl(3R)-4-{[1-(2-aminophenyl)-2-phenyl-1H-pyrrol-3-yl]carbonyl)-3-benzylpiperazine-1-carboxylate (a 0.02 M solution in ethyl acetate, 500 μl), (2R)-propylene oxide (a 0.06 M solution in ethyl acetate, 500 μl) and indium tribromide (a 0.06 M solution in ethyl acetate, 500 μl) were mixed, and the mixture was agitated at room temperature for 4 days. To the reaction mixture were added water (2 ml) and ethyl acetate (2 ml), and the reaction mixture was extracted and fractionated with Presep dehydration (manufactured by Wako Pure Chemical Industries, Ltd.). To the aqueous layer was further added ethyl acetate (2 ml) and extracted. The ethyl acetate layers were combined and the reaction mixture was heated and concentrated by a nitrogen spraying apparatus. TFA (a 10% solution in dichloromethane, 3 ml) was added thereto, and the final solution was left to stand. The solvent was removed using a nitrogen spraying apparatus, and the residue was purified with reverse-phase HPLC analysis. The target fraction was heated and concentrated by a nitrogen spraying apparatus through a MP—CO₃H resin (manufactured by Polymer Laboratories Ltd.) to give the desired product (2.0 mg).

MS (ES+) 495 (M+1)

In the same manner as in Example 224, the following compounds (Examples 225 to 235) were obtained.

TABLE 9

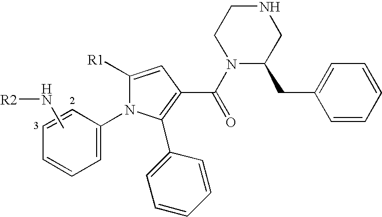

| | | substi- | | |
|---|---|---|---|---|
| | | tuted | | |
| Ex. No. | R1 | position | R2 | MS(ESI+) |
| 225 | H | 2- | 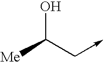 | 495 |
| 226 | H | 2- | 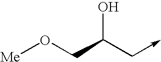 | 525 |
| 227 | H | 2- | 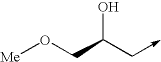 | 525 |
| 228 | H | 2- | 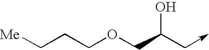 | 567 |
| 229 | H | 2- | 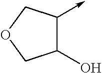 | 523 |
| 230 | Me | 3- | 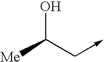 | 509 |
| 231 | Me | 3- | 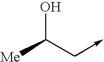 | 509 |
| 232 | Me | 3- | 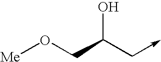 | 539 |
| 233 | Me | 3- | 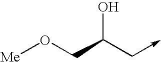 | 539 |
| 234 | Me | 3- | 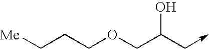 | 581 |
| 235 | Me | 3- | 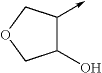 | 537 |

In the same manner as in the methods shown in the above-mentioned Example 1 (Method A)-Example 10 (Method J), the following compounds (Example 236-451) were obtained. The respective compounds were isolated and purified as necessary by a known means such as phase transfer, liquid conversion, solvent extraction, silica gel column chromatography, reversed-phase preparative HPLC and the like. However, the compounds indicated with “-” instead of “salt” in Tables were isolated by Method A to Method I as crystals or an amorphous solid of the free compound by omitting the treatment with a 4 N hydrogen chloride-ethyl acetate solution in the final process.

TABLE 10

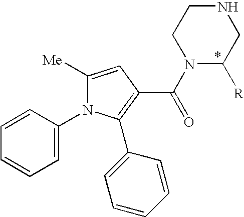

| | | chiral | synthetic | | |
|---|---|---|---|---|---|
| Ex. No. | R | center | method | salt | MS(ESI+) |
| 236 | 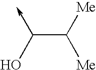 | S | G | — | 418 |
| 237 | 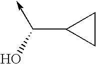 | S | I | — | 416 |
| 238 | 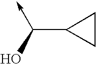 | S | I | — | 416 |
| 239 | 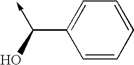 | S | J | — | 452 |
| 240 | 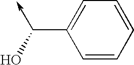 | S | J | HCl | 452 |
| 241 | 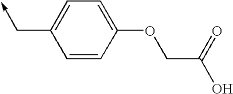 | R | B | — | 510 |
| 242 | 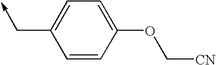 | R | I | — | 491 |
| 243 | 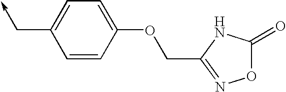 | R | I | — | 550 |
| 244 | 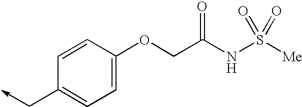 | R | G | — | 587 |
| 245 | 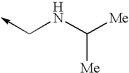 | S | J | 2HCl | 417 |
| 246 | CH2CN | racemic | I | — | 385 |
| 247 | 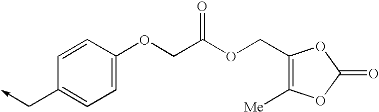 | R | J | HCl | 622 |
| 248 | 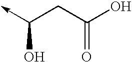 | S | J | HCl | 490 |
| 249 | 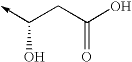 | S | J | TFA | 490 |
| 250 | 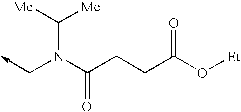 | S | J | HCl | 545 |
| 251 | 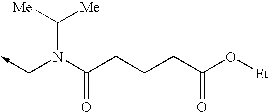 | S | J | HCl | 559 |
| 252 | 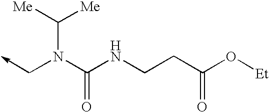 | S | J | HCl | 560 |
| 253 | 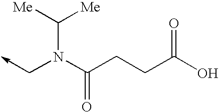 | S | J | HCl | 517 |
| 254 | 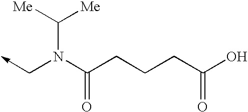 | S | J | HCl | 531 |
| 255 | 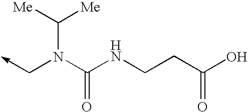 | S | J | HCl | 532 |
| 256 | 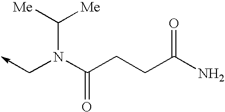 | S | J | HCl | 516 |
| 257 | 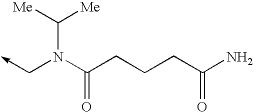 | S | J | HCl | 530 |
| 258 | 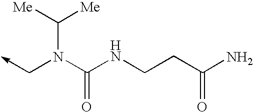 | S | J | HCl | 531 |
| 259 | 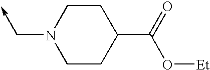 | S | J | 2HCl | 515 |
| 260 | 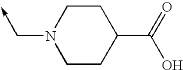 | S | J | 2HCl | 487 |
| 261 | 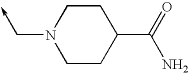 | S | J | 2HCl | 486 |
| 262 | 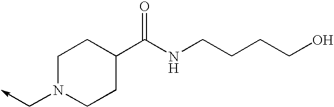 | S | J | 2HCl | 558 |
| 263 | 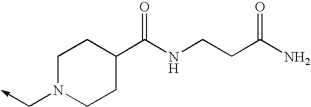 | S | J | 2HCl | 557 |
| 264 | 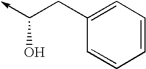 | S | G | — | 466 |
| 265 | 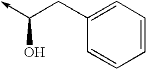 | S | G | — | 466 |
| 266 | 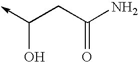 | S | I | HCl | 433 |
| 267 | 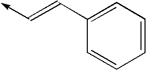 | R | J | HCl | 448 |
| 268 | 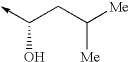 | S | I | — | 432 |
| 269 | 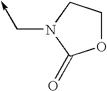 | S | I | — | 445 |
| 270 | 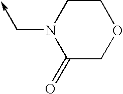 | S | I | — | 459 |
| 271 | 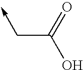 | racemic | J | HCl | 404 |
| 272 | 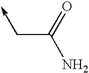 | racemic | J | HCl | 403 |
| 273 | 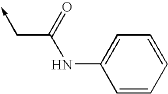 | racemic | J | HCl | 479 |
| 274 | 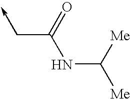 | racemic | J | HCl | 445 |
| 275 | Ph | racemic | D | — | 422 |
| 276 | 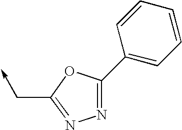 | racemic | J | HCl | 504 |
| 277 | 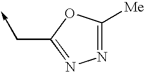 | racemic | J | HCl | 442 |
| 278 | 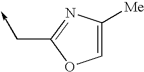 | racemic | G | HCl | 441 |
| 279 | 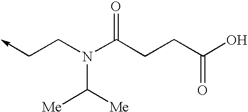 | racemic | G | — | 531 |
| 280 | 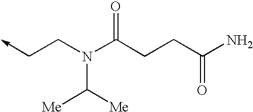 | racemic | G | — | 530 |
| 281 | 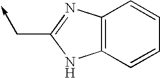 | racemic | G | — | 476 |
| 282 | 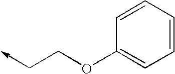 | racemic | G | HCl | 466 |
| 283 | 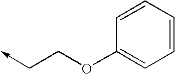 | R | B | 2HCl | 466 |

TABLE 11

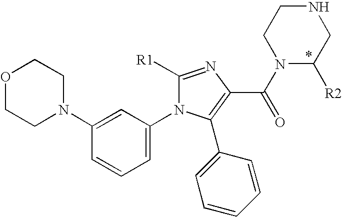

| | | | chiral | synthetic | | |
|---|---|---|---|---|---|---|
| Ex. No. | R1 | R2 | center | method | salt | MS(ESI+) |
| 284 | H | 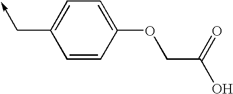 | R | B | — | 582 |
| 285 | H | 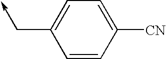 | R | H | — | 533 |
| 286 | H | 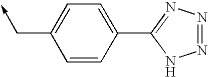 | R | G | — | 576 |
| 287 | H | 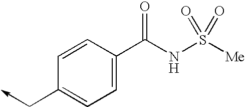 | R | G | — | 629 |
| 288 | H | 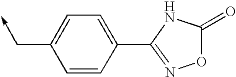 | R | I | — | 592 |
| 289 | H | 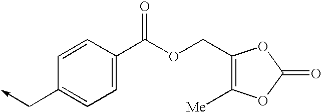 | R | J | 2HCl | 664 |
| 290 | H | 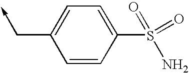 | R | D | — | 587 |
| 291 | Me | CH2Ph | R | I | — | 522 |
| 292 | H | 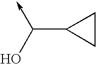 | S | B | — | 488 |
| 293 | H | 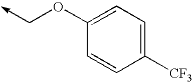 | S | D | — | 582 |
| 294 | H | CH2OH | S | B | — | 448 |
| 295 | H | 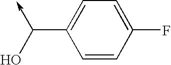 | S | B | — | 542 |
| 296 | H | 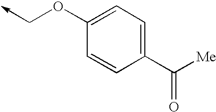 | S | I | — | 566 |
| 297 | H | 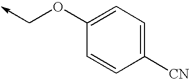 | S | D | — | 549 |
| 298 | H | 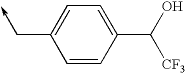 | R | D | — | 606 |
| 299 | H | 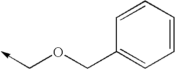 | S | G | — | 538 |
| 300 | H | 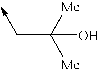 | recemic | D | — | 490 |
| 301 | H | 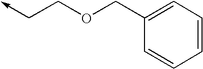 | recemic | D | — | 552 |
| 302 | H | 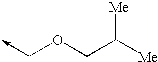 | S | G | — | 502 |
| 303 | H | 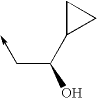 | R | G | — | 502 |
| 304 | H | 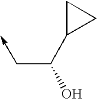 | R | G | — | 502 |
| 305 | H | 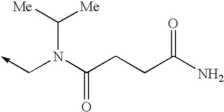 | S | B | 2HCl | 588 |
| 306 | H | 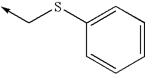 | recemic | D | — | 540 |
| 307 | H | 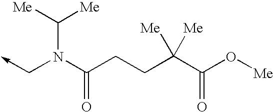 | S | B | 2HCl | 645 |
| 308 | H | 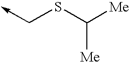 | S | D | — | 506 |
| 309 | H | 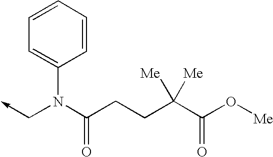 | S | B | 2HCl | 679 |
| 310 | H | 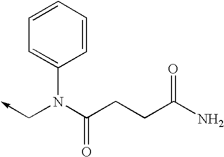 | S | B | 2HCl | 622 |
| 311 | OEt | CH2Ph | R | I | — | 552 |

TABLE 12

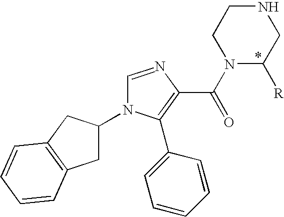

| | | chiral | synthetic | | |
|---|---|---|---|---|---|
| Ex. No. | R | center | method | salt | MS(ESI+) |
| 312 | 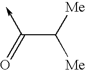 | S | G | — | 443 |
| 313 | 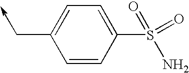 | R | D | — | 542 |
| 314 | 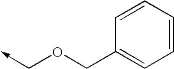 | S | G | — | 493 |
| 315 | 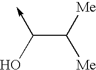 | S | I | — | 445 |
| 316 | 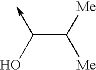 | S | I | HCl | 445 |
| 317 | 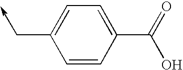 | R | G | — | 507 |
| 318 | iBu | R | G | — | 429 |
| 319 | 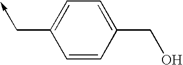 | R | D | — | 493 |
| 320 | 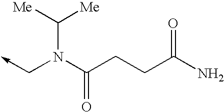 | S | J | HCl | 543 |
| 321 | 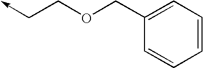 | recemic | D | — | 507 |
| 322 | 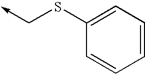 | recemic | D | — | 495 |
| 323 | 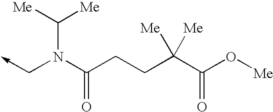 | S | B | HCl | 600 |
| 324 | 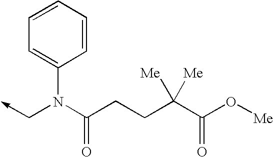 | S | B | HCl | 634 |
| 325 | 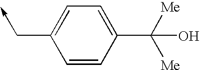 | R | G | — | 521 |
| 326 | 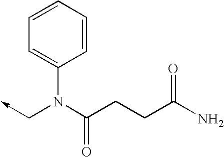 | S | B | HCl | 577 |
| 327 | 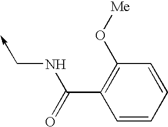 | S | B | HCl | 536 |
| 328 | 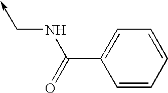 | S | B | HCl | 506 |
| 329 | 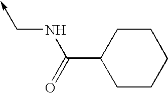 | S | B | HCl | 512 |
| 330 | 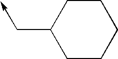 | R | B | — | 469 |
| 331 | 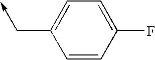 | R | B | HCl | 481 |
| 332 | 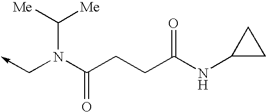 | S | B | HCl | 583 |
| 333 | 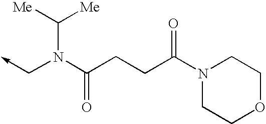 | S | B | HCl | 613 |
| 334 | 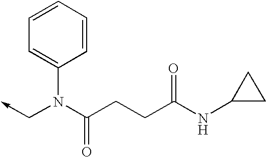 | S | B | HCl | 617 |
| 335 | 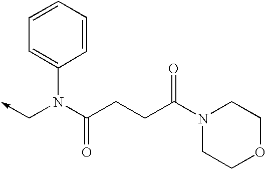 | S | B | HCl | 647 |
| 336 | 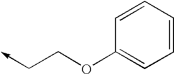 | R | B | 2HCl | 493 |

TABLE 13

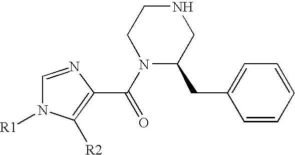

| | | | synthetic | | |
|---|---|---|---|---|---|
| Ex. No. | R1 | R2 | method | salt | MS(ESI+) |
| 337 | 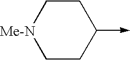 | Ph | I | — | 444 |
| 338 | 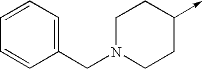 | Ph | I | — | 520 |
| 339 | 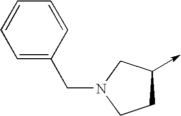 | Ph | I | — | 506 |
| 340 | 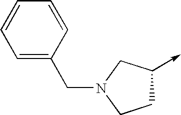 | Ph | I | — | 506 |
| 341 | 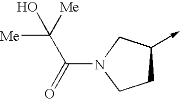 | Ph | I | — | 502 |
| 342 | 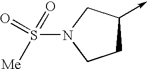 | Ph | I | — | 494 |
| 343 | 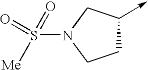 | Ph | I | — | 494 |
| 344 | 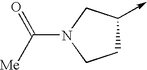 | Ph | I | — | 457 |
| 345 | 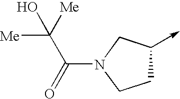 | Ph | I | — | 502 |
| 346 | Ph | 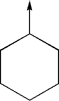 | D | — | 429 |
| 347 | Ph | 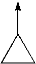 | D | — | 387 |
| 348 | 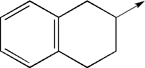 | Ph | D | — | 477 |
| 349 | 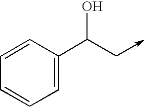 | Ph | D | — | 467 |
| 350 | 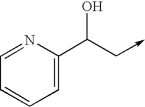 | Ph | D | — | 468 |
| 351 | 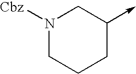 | Ph | I | — | 564 |
| 352 | 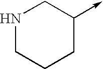 | Ph | I | — | 430 |
| 353 | 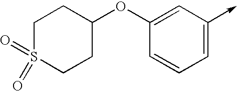 | Ph | I | — | 571 |
| 354 | 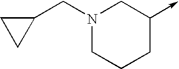 | Ph | I | — | 484 |
| 355 | 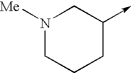 | Ph | I | — | 444 |
| 356 | 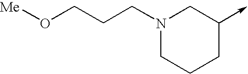 | Ph | I | — | 502 |
| 357 | 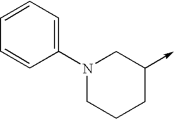 | Ph | I | — | 506 |
| 358 | 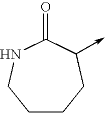 | Ph | I | — | 458 |
| 359 | 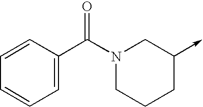 | Ph | I | — | 534 |
| 360 | 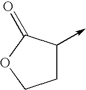 | Ph | D | HCl | 431 |
| 361 | 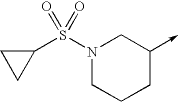 | Ph | I | — | 534 |
| 362 | 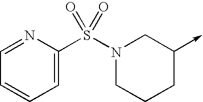 | Ph | I | — | 571 |
| 363 | 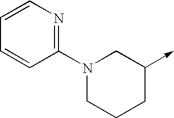 | Ph | I | — | 507 |
| 364 | 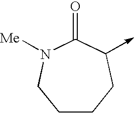 | Ph | I | — | 472 |
| 365 | 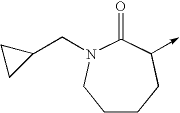 | Ph | I | — | 512 |
| 366 | 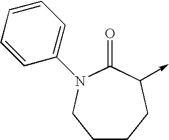 | Ph | I | — | 534 |
| 367 | 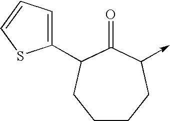 | Ph | I | — | 540 |
| 368 | 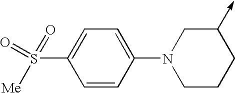 | Ph | I | — | 584 |
| 369 | 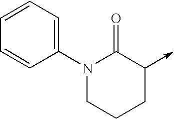 | Ph | I | — | 520 |
| 370 | 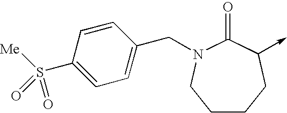 | Ph | I | — | 626 |
| 371 | 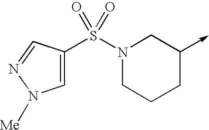 | Ph | I | — | 574 |
| 372 | 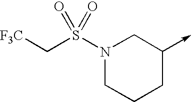 | Ph | I | — | 576 |
| 373 | 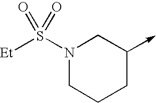 | Ph | I | — | 522 |
| 374 | 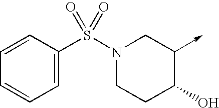 | Ph | D | — | 586 |
| 375 | 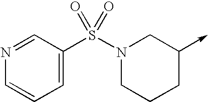 | Ph | I | — | 571 |
| 376 | 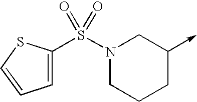 | Ph | I | — | 576 |
| 377 | 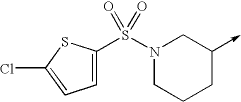 | Ph | I | — | 611 |
| 378 | 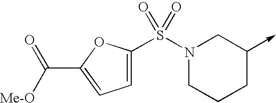 | Ph | I | — | 618 |
| 379 | 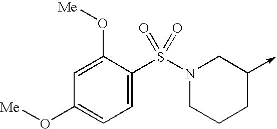 | Ph | I | — | 630 |
| 380 | 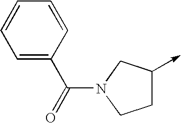 | Ph | I | — | 520 |
| 381 | 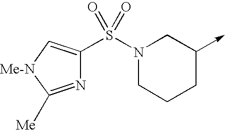 | Ph | I | — | 588 |
| 382 | 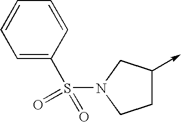 | Ph | I | — | 556 |
| 383 | 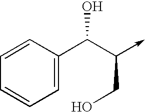 | Ph | I | — | 497 |
| 384 | 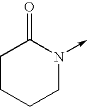 | Ph | I | — | 444 |
| 385 | 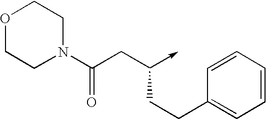 | Ph | E | HCl | 592 |
| 386 | 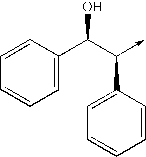 | Ph | I | — | 543 |
| 387 | 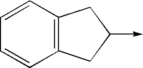 | 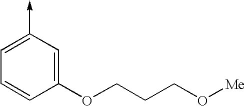 | E | HCl | 551 |
| 388 | 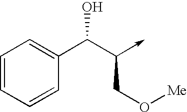 | Ph | I | — | 511 |
| 389 | 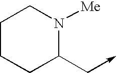 | Ph | I | — | 458 |
| 390 | 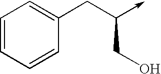 | Ph | I | — | 481 |
| 391 | 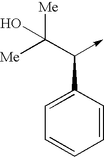 | Ph | I | — | 495 |
| 392 | 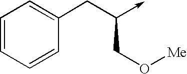 | Ph | I | — | 495 |
| 393 | 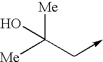 | Ph | J | HCl | 419 |
| 394 | 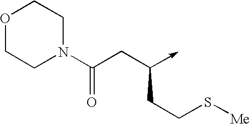 | Ph | E | HCl | 562 |
| 395 | 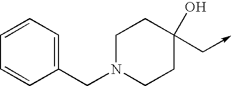 | Ph | J | HCl | 550 |
| 396 | 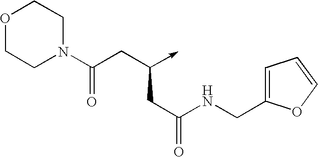 | Ph | E | HCl | 625 |
| 397 | 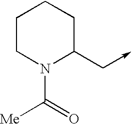 | Ph | I | — | 486 |
| 398 | 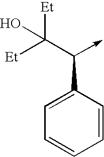 | Ph | I | — | 523 |
| 399 | 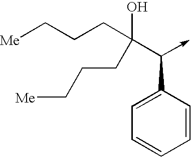 | Ph | I | — | 579 |
| 400 | 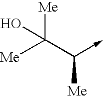 | Ph | I | HCl | 433 |
| 401 | 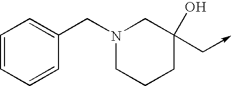 | Ph | J | 2HCl | 550 |
| 402 | 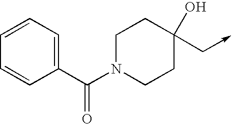 | Ph | J | HCl | 564 |
| 403 | 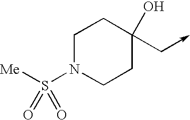 | Ph | J | HCl | 538 |
| 404 | 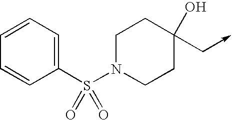 | Ph | J | HCl | 600 |
| 405 | 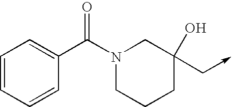 | Ph | J | HCl | 564 |
| 406 | 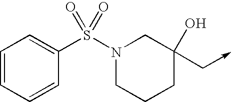 | Ph | J | HCl | 600 |
| 407 | 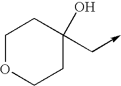 | Ph | J | HCl | 461 |
| 408 | 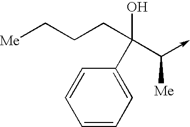 | Ph | I | HCl | 537 |
| 409 | 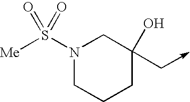 | Ph | J | — | 538 |
| 410 | 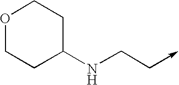 | Ph | G | 2HCl | 474 |
| 411 | 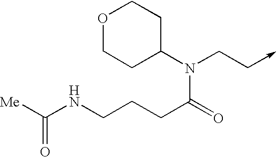 | Ph | G | HCl | 601 |
| 412 | 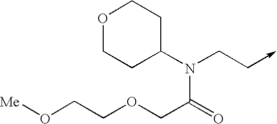 | Ph | G | HCl | 590 |
| 413 | 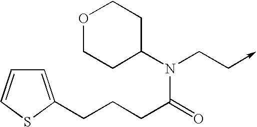 | Ph | H | HCl | 626 |
| 414 | 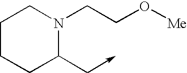 | Ph | G | — | 502 |
| 415 | 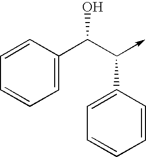 | Ph | I | 2HCl | 543 |
| 416 | 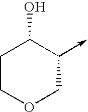 | Ph | I | — | 447 |

TABLE 14

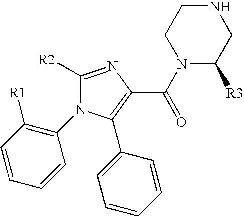

| | | | | synthetic | | |
|---|---|---|---|---|---|---|
| Ex. No. | R1 | R2 | R3 | method | salt | MS(ESI+) |
| 417 | H | OEt | CH2Ph | D | — | 467 |
| 418 | H | Cl | CH2Ph | I | — | 457 |
| 419 | OMe | OEt | 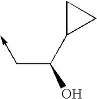 | G | — | 491 |
| 420 | OMe | OEt | 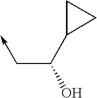 | G | — | 491 |
| 421 | H | OEt | 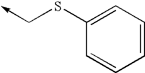 | I | — | 499 |
| 422 | H | OEt | 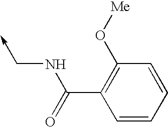 | B | HCl | 540 |
| 423 | H | OEt | 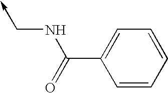 | B | HCl | 510 |
| 424 | H | OEt | 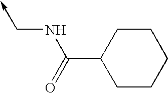 | B | HCl | 516 |
| 425 | H | OEt | 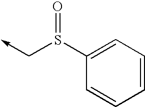 | I | — | 515 |
| 426 | H | OEt | 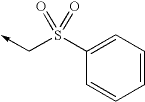 | I | — | 531 |
| 427 | H | OEt | 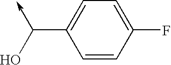 | B | — | 501 |
| 428 | H | OEt | 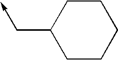 | B | — | 473 |
| 429 | H | OEt | 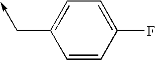 | B | — | 485 |
| 430 | Me | OMe | CH2Ph | D | — | 467 |
| 431 | H | OEt | 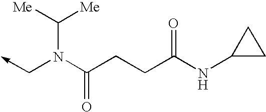 | B | — | 587 |
| 432 | H | OEt | 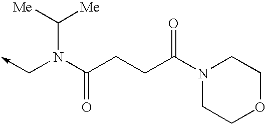 | B | — | 617 |
| 433 | H | OEt | 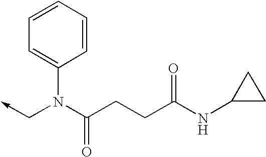 | B | — | 621 |
| 434 | H | OEt | 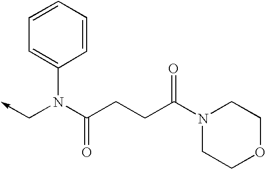 | B | — | 651 |
| 435 | NO2 | OMe | CH2Ph | D | — | 498 |
| 436 | H | OEt | 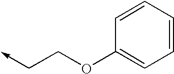 | B | — | 497 |

TABLE 15

| | |
|---|---|
| 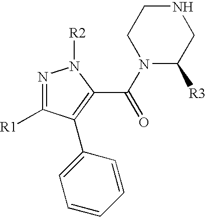 | 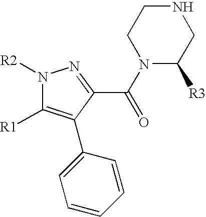 |
| (X type) | (Y type) |

| | substi- | | | | | | |
|---|---|---|---|---|---|---|---|
| | tution | | | | synthetic | | |
| Ex. No. | type | R1 | R2 | R3 | method | salt | MS(ESI+) |
| 437 | X | Ph | H | CH2Ph | I | — | 423 |
| 438 | X | Ph | PhCH2 | CH2Ph | D | HCl | 513 |
| 439 | X | Ph | 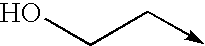 | CH2Ph | I | HCl | 467 |
| 440 | Y | Ph | 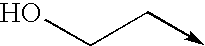 | CH2Ph | I | — | 467 |
| 441 | X | Ph | H | 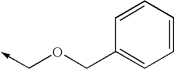 | G | — | 453 |
| 442 | X | Ph | H | iBu | G | — | 389 |
| 443 | X | Ph | 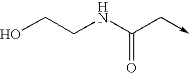 | CH2Ph | J | — | 524 |
| 444 | Y | Ph | 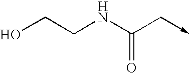 | CH2Ph | J | — | 524 |
| 445 | Y | Ph | 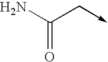 | CH2Ph | J | — | 480 |
| 446 | X | 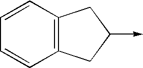 | H | CH2Ph | D | — | 463 |
| 447 | X | 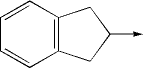 | H | 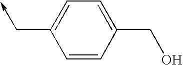 | D | — | 493 |
| 448 | X | 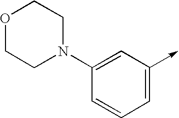 | H | CH2Ph | D | — | 508 |
| 449 | X | Ph | 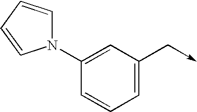 | CH2Ph | D | — | 578 |
| 450 | X | Ph | PhCH2 | 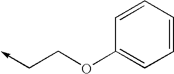 | B | 2HCl | 543 |
| 451 | X | 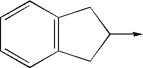 | H | 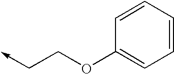 | B | 2HCl | 493 |

In the same manner as in Example 2 (Method B), the following compound (Example 452) was obtained.

### Example 452

### (1R,2S)-2-(4-{[(2R)-2-(2-Phenoxyethyl)piperazin-1-yl]carbonyl}-5-phenyl-1H-imidazol-1-yl)-1,2-diphenylethanol dihydrochloride

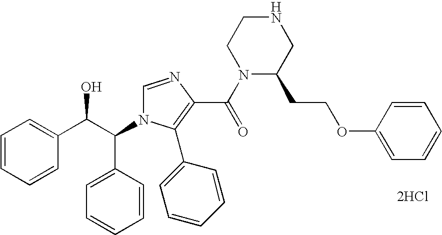

MS (ESI+, m/e) 573 (M+1)

In the same manner as in Example 4 (Method D), the following compounds (Examples 453 to 457) were obtained. However, the final product was isolated as an amorphous solid of the free compound without treatment by a 4 N hydrogen chloride-ethyl acetate solution.

### Example 453

### (2R)-2-Benzyl-1-{[5-cyclohexyl-1-(3-methoxyphenyl)-1H-pyrazol-4-yl]carbonyl}piperazine

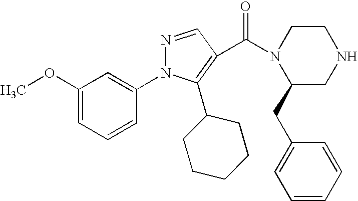

MS (ESI+, m/e) 459 (M+1)

### Example 454

### (2R)-2-Benzyl-1-{(4-(3-bromophenyl)-3-phenyl-1H-pyrrol-2-yl]carbonyl}piperazine

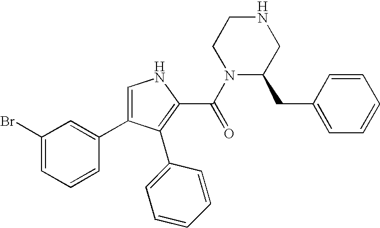

MS (ESI+, m/e) 500 (M+1)

### Example 455

### (R)-2-Benzyl-1-{[1-(2,3-dihydro-1H-inden-2-yl)-5-phenyl-1H-pyrazol-4-yl]carbonyl}piperazine

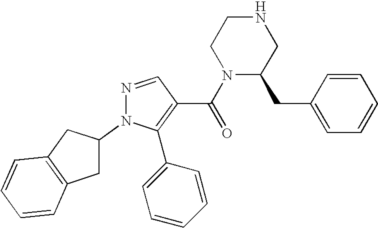

MS (ESI+, m/e) 463 (M+1)

### Example 456

### (2R)-2-Benzyl-1-[(3,4-diphenylpyridin-2-yl)carbonyl]piperazine

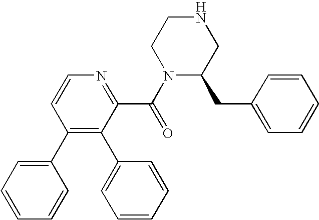

MS (ESI+, m/e) 434 (M+1)

### Example 457

### (2R)-2-Benzyl-1-{[4-(3-bromophenyl)-3-phenyl-1-(1,3-thiazol-4-ylmethyl)-1H-pyrrol-2-yl]carbonyl}piperazine

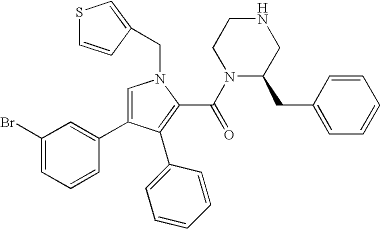

MS (ESI+, m/e) 597 (M+1)

In the same manner as in Example 7 (Method G), the following compounds (Examples 458 to 461) were obtained. However, the final product was isolated as an amorphous solid of the free compound without treatment by a 4 N hydrogen chloride-ethyl acetate solution.

### Example 458

### (2R)-2-Benzyl-1-[(4,5-diphenyl-4H-1,2,4-triazol-3-yl)carbonyl]piperazine

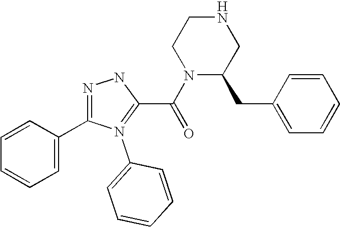

MS (ESI+, m/e) 424 (M+1)

### Example 459

### {(2S,6R)-6-Benzyl-1-[(5-methyl-1,2-diphenyl-1H-pyrrol-3-yl)carbonyl]piperazin-2-yl)methanol

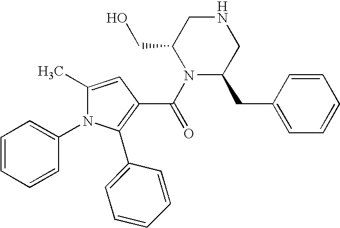

MS (ESI+, m/e) 466 (M+1)

### Example 460

### ((2S,6R)-6-Benzyl-1-{[1-(3-morpholinophenyl)-5-phenyl-1H-imidazol-4-yl]carbonyl}piperazin-2-yl)methanol

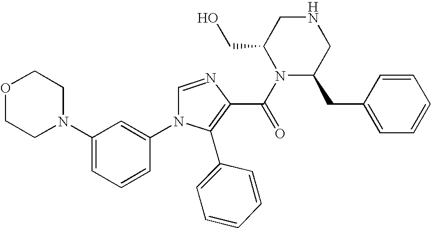

MS (ESI+, m/e) 538 (M+1)

### Example 461

### (2R,6R)-2-Benzyl-6-methyl-1-[(5-methyl-1,2-diphenyl-1H-pyrrol-3-yl)carbonyl]piperazine

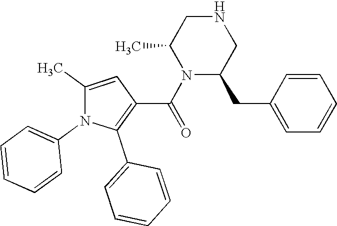

MS (ESI+, m/e) 480 (M+1)

In the same manner as in Example 9 (Method I), the following compounds (Examples 462 to 464) were obtained. However, the final product was isolated as an amorphous solid of the free compound without treatment by a 4 N hydrogen chloride-ethyl acetate solution.

### Example 462

### 4-[3-(4-{[(2R)-2-Benzylpiperazin-1-yl]carbonyl}-5-cyclohexyl-1H-pyrazol-1-yl)phenyl]morpholine

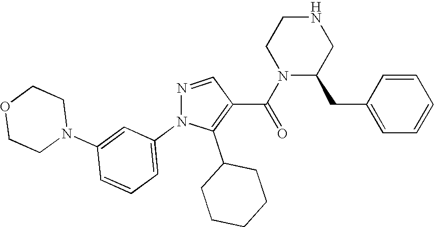

MS (ESI+, m/e) 514 (M+1)

### Example 463

### 5-{[(2R)-2-Benzylpiperazin-1-yl]carbonyl}-3-(3-bromophenyl)-4-phenyl-1H-pyrrole-2-carbaldehyde

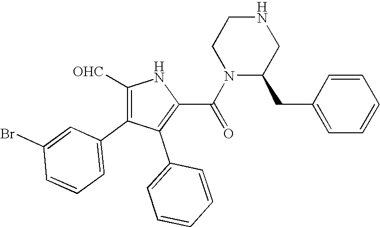

MS (ESI+, m/e) 528 (M+1)

### Example 464

### (2R)-2-Benzyl-1-({2-ethoxy-1-[3-(methylsulfonyl)phenyl]-5-phenyl-1H-imidazol-4-yl}carbonyl)piperazine

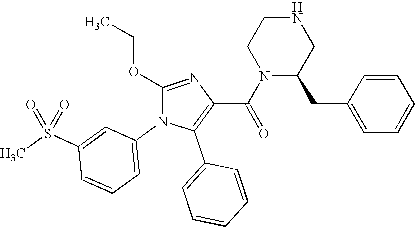

MS (ESI+, m/e) 545 (M+1)

In the same manner as in Example 174, the following compound (Example 465) was obtained.

### Example 465

### 4-({(2S)-1-[(5-Methyl-1,2-diphenyl-1H-pyrrol-3-yl)carbonyl]piperazin-2-yl}methyl)morpholine dihydrochloride

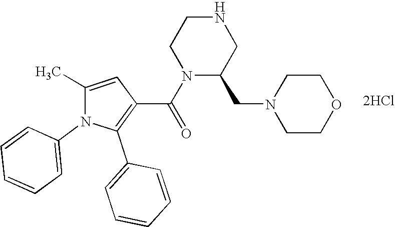

MS (ESI+, m/e) 445 (M+1)

### Example 466

### [5-{[(2R)-2-Benzylpiperazin-1-yl]carbonyl}-3-(3-bromophenyl)-4-phenyl-1H-pyrrol-2-yl]methanol

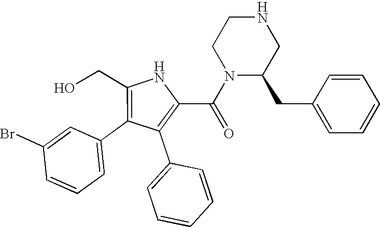

5-{[(2R)-2-Benzylpiperazin-1-yl]carbonyl}-3-(3-bromophenyl)-4-phenyl-1H-pyrrole-2-carbaldehyde (compound of Example 463) (300 mg) was dissolved in methanol (10 ml) and the mixture was ice-cooled. Sodium borohydride (21 mg) was added, and the mixture was stirred at room temperature for 1 hr. An ammonium chloride aqueous solution was added to the reaction mixture, and the mixture was basified with an aqueous potassium carbonate solution and extracted with ethyl acetate. The extract was washed with brine, and dried over anhydrous magnesium sulfate. The solvent was evaporated in vacuo and the residue was vacuum dried to give the desired product (80 mg).

MS (ESI+, m/e) 530 (M+1)

### Example 467

### 4-{[5-{[(2R)-2-Benzylpiperazin-1-yl]carbonyl}-3-(3-bromophenyl)-4-phenyl-1H-pyrrol-2-yl]methyl}morpholine

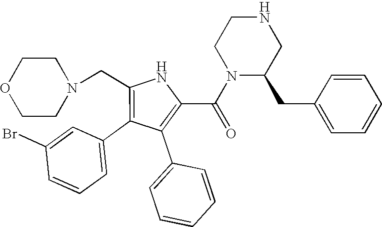

tert-Butyl(3R)-3-benzyl-4-{[4-(3-bromophenyl)-5-formyl-3-phenyl-1H-pyrrol-2-yl]carbonyl}piperazine-1-carboxylate (100 mg) and morpholine (50 mg) were dissolved in 1,2-dichloroethane (10 ml) and the mixture was ice-cooled. Acetic acid (0.1 ml) and sodium triacetoxyborohydride (220 mg) were added, and the mixture was stirred at room temperature for 15 hr. The reaction mixture was basified with an aqueous potassium carbonate solution, and extracted with 1,2-dichloroethane. The extract was washed with brine, dried over anhydrous magnesium sulfate, and concentrated in vacuo. The residue was dissolved in TFA (5 ml) and, after stirring for 30 min, the mixture was basified by adding an aqueous potassium carbonate solution by small portions, and extracted with 1,2-dichloroethane. The extract was washed with brine, dried over anhydrous magnesium sulfate, and concentrated in vacuo. The residue was subjected to silica gel column chromatography, and the target fraction was concentrated in vacuo to give the desired product (23 mg).

MS (ESI+, m/e) 599 (M+1)

### Example 468

### 1-[5-{[(2R)-2-Benzylpiperazin-1-yl]carbonyl}-3-(3-bromophenyl)-4-phenyl-1H-pyrrol-2-yl]methylamine

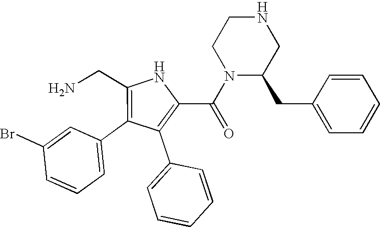

tert-Butyl(3R)-3-benzyl-4-{[4-(3-brothophenyl)-5-formyl-3-phenyl-1H-pyrrol-2-yl]carbonyl}piperazine-1-carboxylate (100 mg) and ammonium acetate (50 mg) were dissolved in 1,2-dichloroethane (10 ml) and the mixture was ice-cooled. Acetic acid (0.1 ml) and sodium triacetoxyborohydride (220 mg) were added, and the mixture was stirred at room temperature for 15 hr. The reaction mixture was basified with an aqueous potassium carbonate solution, and extracted with 1,2-dichloroethane. The extract was washed with brine, dried over anhydrous magnesium sulfate, and concentrated in vacuo. The residue was dissolved in TFA (5 ml) and, after stirring for 30 min, the mixture was basified by adding an aqueous potassium carbonate solution by small portions, and extracted with 1,2-dichloroethane. The extract was washed with brine, dried over anhydrous magnesium sulfate, and concentrated in vacuo. The residue was subjected to silica gel column chromatography, and the target fraction was concentrated in vacuo to give the desired product (8 mg).

MS (ESI+, m/e) 529 (M+1)

### Example 469

### 4-[3-(4-{[(2R)-2-(4-Methylbenzyl)piperazin-1-yl]carbonyl}-5-phenyl-1H-imidazol-1-yl)phenyl]morpholine

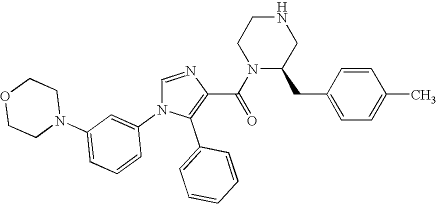

4-[((2R)-4-Benzyl-1-{[1-(3-morpholinophenyl)-5-phenyl-1H-imidazol-4-yl]carbonyl}piperazin-2-yl)methyl]benzonitrile (311 mg) was dissolved in methanol (10 ml), 20% palladium hydroxide on carbon (containing 50% water) (100 mg) was added, and a catalytic hydrogenation was performed at room temperature and atmospheric pressure for 62 hr. The catalyst was filtered off, and the filtrate was concentrated in vacuo to give the desired product (157 mg) as an amorphous solid.

MS (ESI+, m/e) 522 (M+1)

### Example 470

### 4-[3-(5-Phenyl-4-{[(2R)-2-(2-pyridin-2-ylethyl)piperazin-1-yl]carbonyl}-1H-imidazol-1-yl)phenyl]morpholine

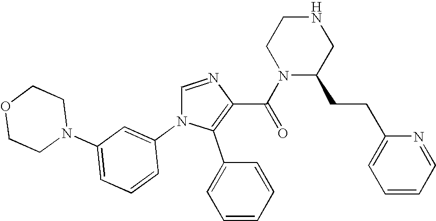

A mixture of 1-(3-morpholinophenyl)-5-phenyl-1H-imidazole-4-carboxylic acid (259 mg), (3R)-1-benzyl-3-[(E)-2-pyridin-2-ylvinyl]piperazine dihydrochloride (260 mg), WSC.HCl (168 mg), HOBt (119 mg), triethylamine (720 μl) and DMF (10 ml) was stirred at room temperature for 8 hr, and poured into brine, and the mixture was extracted with ethyl acetate. The extract was dried over anhydrous magnesium sulfate, and the solvent was evaporated in vacuo. The residue was subjected to basic silica gel column chromatography, and the fraction eluted with ethyl acetate-hexane-methanol (1:1:0 to 20:0:1) was concentrated in vacuo to give an amorphous solid (273 mg). The total amount thereof was dissolved in methanol (15 ml), 20% palladium hydroxide on carbon (containing 50% water) (50 mg) was added, and a catalytic hydrogenation was performed at room temperature and atmospheric pressure for 3 days. The catalyst was filtered off, the filtrate was concentrated in vacuo, and the crystals were collected by filtration to give the desired product (146 mg).

MS (ESI+, m/e) 523 (M+1)

### Example 471

### (S)-((2S)-1-{[1-(2,3-Dihydro-1H-inden-2-yl)-5-phenyl-1H-imidazol-4-yl]carbonyl}piperazin-2-yl)(phenyl)methanol

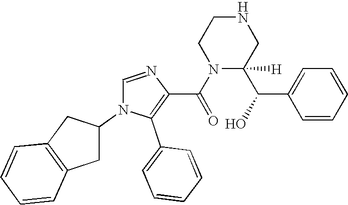

tert-Butyl(3S)-4-{[1-(2,3-dihydro-1H-inden-2-yl)-5-phenyl-1H-imidazol-4-yl]carbonyl}-3-formylpiperazine-1-carboxylate (500 mg) was dissolved in THF (10 ml) and the mixture was cooled to −78° C. Phenylmagnesium bromide (1 M THF solution, 10 ml) was added thereto and the mixture was stirred at the same temperature for 2 hr. A saturated aqueous ammonium chloride solution was added to the reaction mixture, and the mixture was extracted with ethyl acetate. The extract was washed with brine, dried over anhydrous magnesium sulfate, and concentrated in vacuo. The residue was subjected to reversed-phase preparative HPLC (purification conditions are described above), and the target fraction was concentrated in vacuo. TFA (5 ml) were added to the residue and the mixture was stirred at room temperature for 5 min. The reaction mixture was concentrated in vacuo, the residue was subjected to reversed-phase preparative HPLC (purification conditions are described above), and the target fraction was neutralized with saturated aqueous sodium bicarbonate solution. The mixture was extracted with ethyl acetate,and the extract was dried over anhydrous sodium sulfate. The solvent was evaporated in vacuo and the residue was vacuum dried to give the desired product (20 mg).

MS (ESI+, m/e) 479 (M+1)

### Example 472

### 4-[3-(4-{[(2S)-2-({[4-(Methylsulfonyl)benzyl]oxy}methyl)piperazin-1-yl]carbonyl}-5-phenyl-1H-imidazol-1-yl)phenyl]morpholine

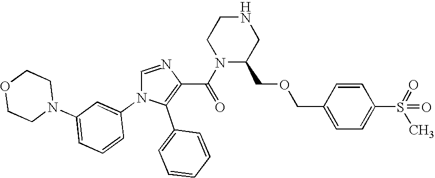

1-(3-Morpholinophenyl)-5-phenyl-1H-imidazole-4-carboxylic acid (168 mg), tert-butyl(3S)-3-({[4-(methylsulfonyl)benzyl]oxy}methyl)piperazine-1-carboxylate (185 mg), WSC.HCl (120 mg), HOBt (22 mg), DMAP (12 mg) and N,N-diisopropylethylamine (124 mg) were dissolved in DMF (3 ml) and, after stirring at room temperature for 15 hr, and the mixture was poured into saturated aqueous sodium bicarbonate solution. The mixture was extracted with ethyl acetate and the extract was washed successively with water and brine, and dried over anhydrous magnesium sulfate. The solvent was evaporated in vacuo. The residue was subjected to basic silica gel column chromatography, and the fraction eluted with ethyl acetate-hexane (1:4 to 3:7) was concentrated in vacuo to give an amorphous solid (300 mg). A 294 mg portion thereof was dissolved in dichloromethane (0.5 ml), TFA (1 ml) was added, and the mixture was stirred at room temperature for 30 min. The reaction mixture was concentrated in vacuo, the residue was neutralized by adding saturated aqueous sodium bicarbonate solution by small portions, and the mixture was extracted with ethyl acetate. The extract was dried over anhydrous magnesium sulfate, and the solvent was evaporated in vacuo to give the desired product (160 mg) as an amorphous solid.

MS (ESI+, m/e) 616 (M+1)

### Example 473

### N-({(2S)-1-[(2-Ethoxy-1,5-diphenyl-1H-imidazol-4-yl)carbonyl]piperazin-2-yl}methyl)-N-isopropylsuccinamide trifluoroacetate

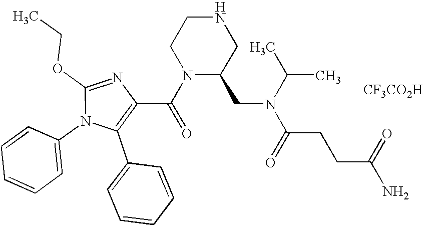

A solution of 2-ethoxy-1,5-diphenyl-1H-imidazole-4-carboxylic acid (154 mg), N-{[(2S)-4-benzylpiperazin-2-yl]methyl}-N-isopropylsuccinamide (191 mg), WSC.HCl (115 mg), HOBt (81 mg) and DMF (3 ml) was stirred at room temperature for 15 hr, and poured into saturated aqueous sodium bicarbonate solution, and the mixture was extracted with ethyl acetate. The extract was washed successively with water and brine, and dried over anhydrous magnesium sulfate. The solvent was evaporated in vacuo. The residue was subjected to basic silica gel column chromatography, and the fraction eluted with ethyl acetate-methanol (1:0 to 20:1) was concentrated in vacuo to give an amorphous form. The obtained amorphous solid (277 mg) was dissolved in methanol (8 ml), 20% palladium hydroxide on carbon (containing 50% water) (140 mg) was added, and a catalytic hydrogenation was performed at room temperature and atmospheric pressure for 15 hr. The catalyst was filtered off, and the filtrate was concentrated in vacuo. The residue was subjected to reversed-phase preparative HPLC (purification conditions are described above), and the target fractions were collected and concentrated in vacuo. The residue was diluted with toluene (about 20 ml), and concentrated again in vacuo. Diethyl ether was added to the residue, and the crystals were collected by filtration to give the desired product (99 mg).

MS (ESI+, m/e) 547 (M+1)

In the same manner as in Example 473, the following compound (Example 474) was obtained.

### Example 474

### N-({(2S)-1-[(2-Ethoxy-1,5-diphenyl-1H-imidazol-4-yl)carbonyl]piperazin-2-yl}methyl)-N-phenylsuccinamide trifluoroacetate

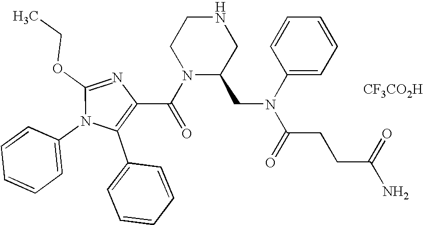

MS (ESI+, m/e) 547 (M+1)

### Example 475

### (2R)-2-Benzyl-1-[(2-methoxy-1,5-diphenyl-1H-imidazol-4-yl)carbonyl]piperazine

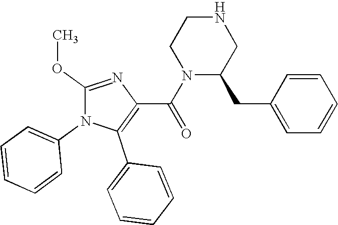

A solution of 2-methoxy-1,5-diphenyl-1H-imidazole-4-carboxylic acid (237 mg), tert-butyl(3R)-3-benzylpiperazine-1-carboxylate (265 mg), WSC.HCl (300 mg), HOBt (60 mg), triethylamine (280 μl) and dichloromethane (7 ml) was stirred at room temperature for 16 hr, and poured into brine, and the mixture was extracted with ethyl acetate. The extract was dried over anhydrous magnesium sulfate, and the solvent was evaporated in vacuo. The residue was subjected to silica gel column chromatography, and the fraction eluted with ethyl acetate-hexane (1:9 to 1:0) was concentrated in vacuo to give an amorphous solid (407 mg). A 401 mg portion thereof was dissolved in dichloromethane (2 ml), TFA (2 ml) was added and, after stirring at room temperature for 3 hr, the mixture was concentrated in vacuo. The residue was dissolved in ethyl acetate, and the solution was washed with saturated aqueous sodium bicarbonate solution, and dried over anhydrous magnesium sulfate. The solvent was evaporated in vacuo to give the desired product (306 mg).

MS (ESI+, m/e) 453 (M+1)

### Example 476

### (2R)-2-Benzyl-1-({5-phenyl-1-[1-(phenylsulfonyl)piperidin-3-yl]-1H-imidazol-4-yl}carbonyl)piperazine

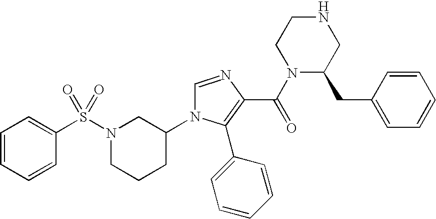

TFA (2 ml) was added to tert-butyl (3R)-3-benzyl-4-({5-phenyl-1-[1-(phenylsulfonyl)piperidin-3-yl]-1H-imidazol-4-yl}carbonyl)piperazine-1-carboxylate (135 mg). After stirring at room temperature for for 5 min, the reaction mixture was concentrated in vacuo. Toluene was added to the residue and the mixture was concentrated again in vacuo. Saturated aqueous sodium bicarbonate solution was added to the residue, and the mixture was extracted with ethyl acetate. The extract was washed with brine, and dried over anhydrous sodium sulfate, and the solvent was evaporated in vacuo to give the desired product (119 mg) as an amorphous solid.

MS (ESI+, m/e) 570 (M+1)

### Example 477

### (2R)-2-Benzyl-1-[(1-{1-[(6-methoxypyridin-3-yl)sulfonyl]piperidin-3-yl}-5-phenyl-1H-imidazol-4-yl)carbonyl]piperazine

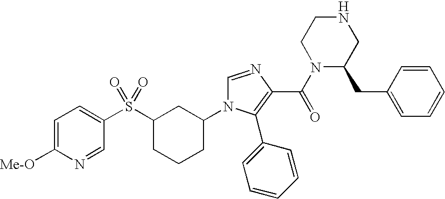

tert-Butyl(3R)-3-benzyl-4-[(1-{1-[(6-methoxypyridin-3-yl)sulfonyl]piperidin-3-yl}-5-phenyl-1H-imidazol-4-yl)carbonyl]piperazine-1-carboxylate (150 mg) was dissolved in chloroform (3 ml), and TFA (2 ml) was added. After stirring at room temperature for 30 min, the mixture was poured into saturated aqueous sodium bicarbonate solution (100 ml), and the mixture was extracted with chloroform. The extract was concentrated in vacuo, the residue was subjected to silica gel column chromatography, and the fraction eluted with ethyl acetate-methanol (1:1) was concentrated in vacuo to give the desired product (120 mg) as an amorphous solid.

MS (ESI+, m/e) 601 (M+1)

### Example 478

### 4-[(3S)-3-(4-{[(2R)-2-Benzylpiperazin-1-yl]carbonyl}-5-phenyl-1H-imidazol-1-yl)-5-phenylpentanoyl]morpholine hydrochloride

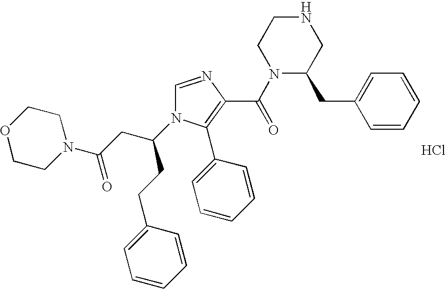

A solution of 1-[(1S)-3-morpholino-3-oxo-1-(2-phenylethyl)propyl]-5-phenyl-1H-imidazole-4-carboxylic acid (506 mg), tert-butyl (3R)-3-benzylpiperazine-1-carboxylate (339 mg), WSC.HCl (269 mg), HOBt (189 mg) and DMF (8 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo. The residue was subjected to silica gel column chromatography, and the fraction eluted with ethyl acetate-hexane-methanol (1:1:0 to 10:0:1) was concentrated in vacuo to give an amorphous solid (460 mg). The total amount thereof was dissolved in ethyl acetate (2.15 ml), and a 4 N hydrogen chloride-ethyl acetate solution (2.15 ml) was added. After stirring at room temperature for 2 hr, the precipitated crystals were collected by filtration, and washed with diethyl ether to give the desired product (406 mg).

MS (ESI+, m/e) 592 (M+2)

### Example 479

### Benzyl{(1R)-2-[(2R)-2-benzylpiperazin-1-yl]-1-[(4-{[(2R)-2-benzylpiperazin-1-yl]carbonyl}-5-phenyl-1H-imidazol-1-yl)methyl]-2-oxoethyl}carbamate dihydrochloride

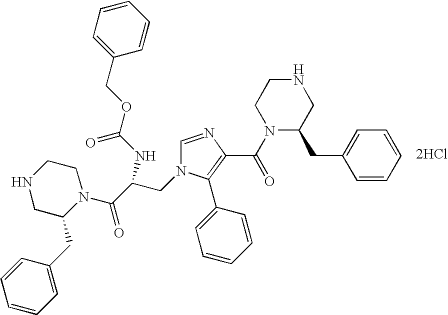

A solution of 1-((2R)-2-{[(benzyloxy)carbonyl]amino}-2-carboxyethyl)-5-phenyl-1H-imidazole-4-carboxylic acid (1.47 g), tert-butyl(3R)-3-benzylpiperazine-1-carboxylate (2.08 g), WSC.HCl (1.65 g), HOBt (1.07 g) and DMF (35 ml) was stirred at room temperature for 15 hr, and poured into water, and the mixture was extracted with ethyl acetate. The extract was washed successively with a 10% aqueous citric acid solution, water, saturated aqueous sodium bicarbonate solution, water and brine, and dried over anhydrous magnesium sulfate. The solvent was evaporated in vacuo. The residue was subjected to silica gel column chromatography, and the fraction eluted with ethyl acetate-methanol (1:0 to 50:1) was concentrated in vacuo to give an amorphous solid (2.48 g). A 350 mg portion thereof was dissolved in ethyl acetate (1.5 ml), and a 4 N hydrogen chloride-ethyl acetate solution (1.5 ml) was added. After stirring at room temperature for 2 hr, the precipitated crystals were collected by filtration, and washed with diethyl ether. This was subjected to reversed-phase preparative HPLC (purification conditions are described above), the target fractions were collected, and diluted with saturated aqueous sodium bicarbonate solution-brine (1:1), and the mixture was extracted with ethyl acetate. The extract was washed with brine, and dried over anhydrous magnesium sulfate. The solvent was evaporated in vacuo. The residue was diluted with diethyl ether (4 ml), a 4 N hydrogen chloride-ethyl acetate solution (180 μl) was added, and the precipitated crystals were collected by filtration to give the desired product (149 mg).

MS (ESI+, m/e) 727 (M+2)

### Example 480

### (2R)-2-Benzyl-1-{[1-({1-[3-(5-methyltetrahydrofuran-2-yl)butyl]piperidin-2-yl}methyl)-5-phenyl-1H-imidazol-4-yl]carbonyl}piperazine

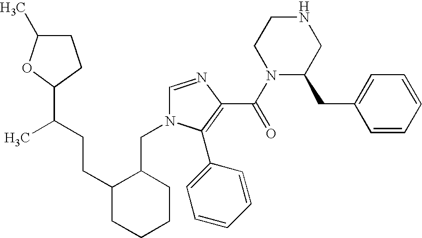

(2R)-2,4-Dibenzyl-1-{[1-({1-[3-(5-methyl-2-furyl)butyl]piperidin-2-yl}methyl)-5-phenyl-1H-imidazol-4-yl]carbonyl}piperazine (125 mg) was dissolved in methanol (5 ml), 20% palladium hydroxide on carbon (containing 50% water) (50 mg) was added, and a catalytic hydrogenation was performed at room temperature and atmospheric pressure for 10 hr. The catalyst was filtered off, the filtrate was concentrated in vacuo, the residue was subjected to reversed-phase preparative HPLC (purification conditions are described above), and the target fractions were collected and concentrated in vacuo. The residue was dissolved in ethyl acetate, and the solution was washed with saturated aqueous sodium bicarbonate solution, and dried over anhydrous magnesium sulfate. The solvent was evaporated in vacuo to give the desired product (61 mg).

MS (ESI+, m/e) 584 (M+1)

### Example 481

### Ethyl5-{[3-(4-{[(2R)-2-benzylpiperazin-1-yl]carbonyl}-5-phenyl-1H-imidazol-1-yl)-2-oxoazepan-1-yl]methyl}furan-2-carboxylate and 5-{[3-(4-{[(2R)-2-benzylpiperazin-1-yl]carbonyl}-5-phenyl-1H-imidazol-1-yl)-2-oxoazepan-1-yl]methyl}furan-2-carboxylic acid trifluoroacetate

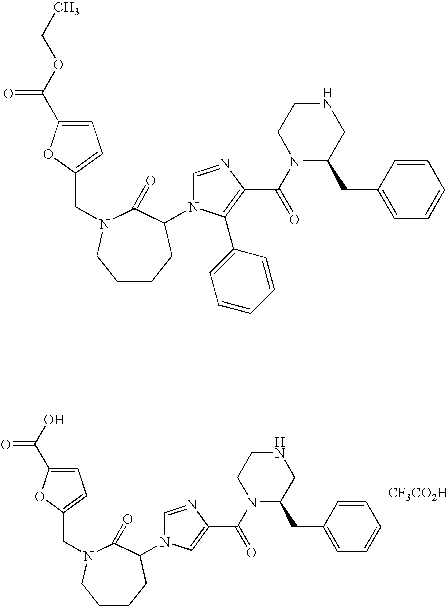

To a mixture (223 mg) of tert-butyl (3R)-3-benzyl-4-{1-[1-(1-{[5-(ethoxycarbonyl)-2-furyl]methyl}-2-oxoazepan-3-yl)-5-phenyl-1H-imidazol-4-yl]vinyl}piperazine-1-carboxylate and 5-{[3-(4-{1-[(2R)-2-benzyl-4-(tert-butoxycarbonyl)piperazin-1-yl]vinyl}-5-phenyl-1H-imidazol-1-yl)-2-oxoazepan-1-yl]methyl}furan-2-carboxylic acid was added TFA (2 ml). After stirring at room temperature for 5 min, the reaction mixture was concentrated in vacuo. The residue was subjected to reversed-phase preparative HPLC (purification conditions are described above), less polar (retention time: long) target fractions were collected, and diluted with saturated aqueous sodium bicarbonate solution and the mixture was extracted with ethyl acetate. The extract was washed with brine, and dried over anhydrous magnesium sulfate. The solvent was evaporated in vacuo to give the desired product (56 mg) of the former. More polar (retention time: short) target fractions were collected, and concentrated in vacuo to give the desired product (94 mg) of the latter.

MS (ESI+, m/e) 610 (M+1), 582 (M+1)

### Example 482

### (2R)-2-Benzyl-1-({1-[(3R,4R)-4-hydroxytetrahydro-2H-pyran-3-yl]-5-phenyl-1H-imidazol-4-yl}carbonyl)piperazine and (2R)-2-benzyl-1-({1-[(3S,4S)-4-hydroxytetrahydro-2H-pyran-3-yl]-5-phenyl-1H-imidazol-4-yl}carbonyl)piperazine

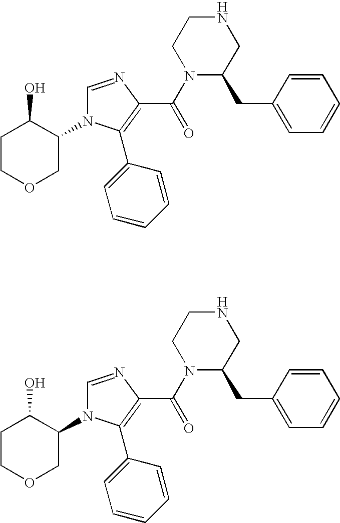

tert-Butyl(3R)-3-benzyl-4-{[5-phenyl-1-(trans-4-hydroxytetrahydro-2H-pyran-3-yl)-1H-imidazol-4-yl]carbonyl}piperazine-1-carboxylate (200 mg) was dissolved in chloroform (2 ml), TFA (2 ml) was added and, after stirring at room temperature for 2 hr, the reaction mixture was concentrated in vacuo. The residue was subjected to reversed-phase preparative HPLC (purification conditions are described above), the target fraction was neutralized with saturated aqueous sodium bicarbonate solution, and the mixture was extracted with ethyl acetate. The extract was dried over anhydrous sodium sulfate, and the solvent was evaporated in vacuo. The residue of a less polar fraction was vacuum dried to give the desired product (45 mg), and the residue of a more polar fraction was vacuum dried to give the desired product (60 mg) each as an amorphous solid.

MS (ESI+, m/e) 447 (M+1)

MS (ESI+, m/e) 447 (M+1)

### Example 483

### (2R)-2-Benzyl-1-({1-[(3R,4S)-4-hydroxy-4-(methoxymethyl)tetrahydro-2H-pyran-3-yl]-5-phenyl-1H-imidazol-4-yl}carbonyl)piperazine and (2R)-2-benzyl-1-({1-[(3S,4R)-4-hydroxy-4-(methoxymethyl)tetrahydro-2H-pyran-3-yl]-5-phenyl-1H-imidazol-4-yl}carbonyl)piperazine

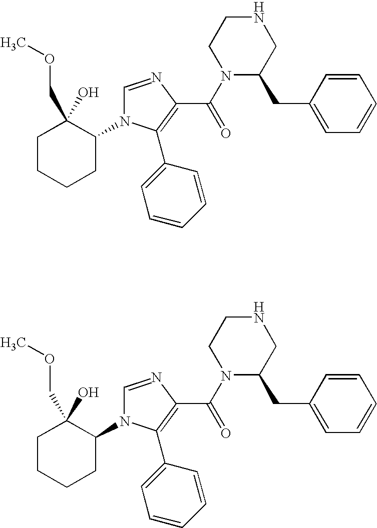

tert-Butyl(3R)-3-benzyl-4-({1-[4-hydroxy-4-(methoxymethyl)tetrahydro-2H-pyran-3-yl]-5-phenyl-1H-imidazol-4-yl)carbonyl)piperazine-1-carboxylate (160 mg) was dissolved in chloroform (3 ml), TFA (3 ml) was added and the mixture was stirred for 1 hr. The reaction mixture was concentrated in vacuo, the residue was subjected to reversed-phase preparative HPLC (purification conditions are described above), the target fraction was neutralized with saturated aqueous sodium bicarbonate solution, and the mixture was extracted with ethyl acetate. The extract anhydrous dried over sodium sulfate, and the solvent was evaporated in vacuo. The residue of a less polar fraction was vacuum dried to give the desired product (60 mg) and the residue of a more polar fraction was vacuum dried to give the desired product (60 mg), each as an amorphous solid.

MS (ESI+, m/e) 491 (M+1)

MS (ESI+, m/e) 491 (M+1)

### Example 484

### Methyl3-(4-{[(2R)-2-benzylpiperazin-1-yl]carbonyl}-5-phenyl-1H-imidazol-1-yl)-4-hydroxy-4-(methoxymethyl)piperidine-1-carboxylate and benzyl3-(4-{[(2R)-2-benzylpiperazin-1-yl]carbonyl}-5-phenyl-1H-imidazol-1-yl)-4-hydroxy-4-(methoxymethyl)piperidine-1-carboxylate

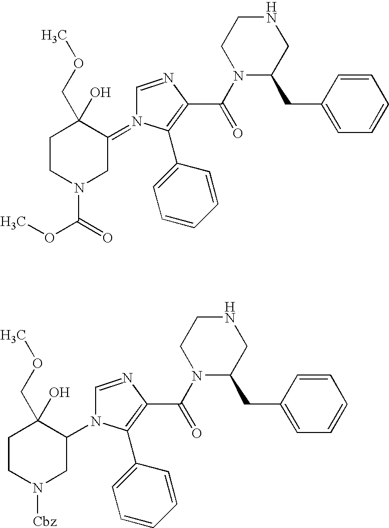

To a mixture (222 mg) of tert-butyl (3R)-3-benzyl-4-({1-[4-hydroxy-1-(methoxycarbonyl)-4-(methoxymethyl)piperidin-3-yl]-5-phenyl-1H-imidazol-4-yl}carbonyl)piperazine-1-carboxylate and tert-butyl (3R)-3-benzyl-4-({1-[1-[(benzyloxy)carbonyl]-4-hydroxy-4-(methoxymethyl)piperidin-3-yl]-5-phenyl-1H-imidazol-4-yl}carbonyl)piperazine-1-carboxylate was added TFA (2 ml). After stirring at room temperature for for 5 min, the reaction mixture was concentrated in vacuo. The residue was subjected to reversed-phase preparative HPLC (purification conditions are described above), the target fractions were collected, and diluted with saturated aqueous sodium bicarbonate solution, and the mixture was extracted with ethyl acetate. The extract was washed with brine, and dried over anhydrous magnesium sulfate. The solvent was evaporated in vacuo to give the former desired product (42 mg) from a more polar (retention time: short) fraction, and the latter desired product (52 mg) from a less polar (retention time: long) fraction.

MS (ESI+, m/e) 548 (M+1), 624 (M+1)

### Example 485

### (2R)-2-Benzyl-1-[(1,5-diphenyl-1H-pyrazol-4-yl)acetyl]piperazine hydrochloride

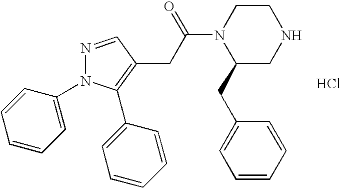

A solution of (1,5-diphenyl-1H-pyrazol-4-yl)acetic acid (358 mg), (3R)-1,3-dibenzylpiperazine (343 mg), WSC.HCl (296 mg), HOBt (209 mg) and DMF (8.5 ml) was stirred at room temperature for 15 hr, and poured into saturated aqueous sodium bicarbonate solution, and the mixture was extracted with ethyl acetate. The, extract was washed successively with water and brine, and dried over anhydrous magnesium sulfate. The solvent was evaporated in vacuo, and the crystals were collected by filtration. The total amount of the obtained crystals (627 mg) was dissolved in methanol-THF (2:1, 18 ml), 20% palladium hydroxide on carbon (containing 50% water) (315 mg) was added, and a catalytic hydrogenation was performed at room temperature and atmospheric pressure for 15 hr. The catalyst was filtered off, the filtrate was concentrated in vacuo, and the residue was subjected to basic silica gel column chromatography, and the fraction eluted with ethyl acetate-methanol (1:0 to 20:1) was concentrated in vacuo. The residue was diluted with diethyl ether (8 ml), 4 N hydrogen chloride-ethyl acetate solution (327 μl) was added, and the precipitated crystals were collected by filtration to give the desired product (270 mg).

MS (ESI+, m/e) 437 (M+1)

### Example 486

### (2R)-2-Benzyl-1-[(1,5-diphenyl-1H-pyrazol-4-yl)sulfonyl]piperazine

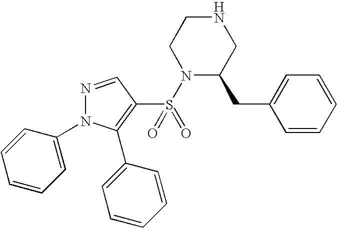

(2R)-2,4-Dibenzyl-1-[(1,5-diphenyl-1H-pyrazol-4-yl)sulfonyl]piperazine (641 mg) was dissolved in methanol-THF (2:1, 18 ml), 20% palladium hydroxide on carbon (containing 50% water) (320 mg) was added, and a catalytic hydrogenation was performed at room temperature and atmospheric pressure for 15 hr. The catalyst was filtered off, the filtrate was concentrated in vacuo, and the crystals were collected by filtration to give the desired product (453 mg).

MS (ESI+, m/e) 459 (M+1)

In the same manner as in Example 486, the following compound (Example 487) was obtained.

### Example 487

### (2R)-2-Benzyl-1-[(1,5-diphenyl-1H-1,2,3-triazol-4-yl)sulfonyl]piperazine

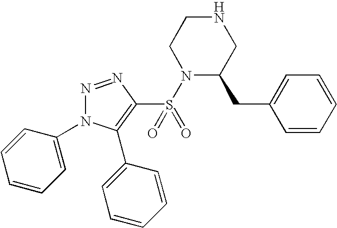

MS (ESI+, m/e) 460 (M+1)

### Preparation Example

### Preparation Example 1

| | |
|---|---|
| (1) Compound of Example 1 | 10.0 g |
| (2) Lactose | 70.0 g |
| (3) Corn starch | 50.0 g |
| (4) Soluble starch | 7.0 g |
| (5) Magnesium stearate | 3.0 g |

10.0 g of the compound of Example 1 and 3.0 g of magnesium stearate are granulated with 70 ml of an aqueous solution of soluble starch (7.0 g as soluble starch), then dry and mix with 70.0 g of lactose and 50.0 g of corn starch (any of lactose, corn starch, soluble starch and magnesium stearate is products in conformity to the 14^{th} revision of the Japanese Pharmacopoeia). The mixture is compressed to give tablets.

### Experimental Examples

Human renin was obtained by expressing preprorenin (1-406) in an animal cell, treating the prorenin (24-406) contained in the culture supernatant with trypsin, and taking the active type (67-406).

### Experimental Example 1

### Construction of Renin Expressing Vector

A plasmid DNA to express human renin in HEK293 cells was prepared as follows. PCR was carried out using human renal cDNA (Clontech Laboratories, Inc., Marathon Ready cDNA) as the template and using two synthetic DNAs (5′-AAGCTTATGGATGGATGGAGA-3′ AND 5′-GGATCCTCAGCGGGCCAAGGC-3′), and the obtained fragments were cloned using a TOPO TA Cloning Kit (Invitrogen Corp.). The obtained fragments were subcloned into pcDNA3.1(+) that had been cleaved by HindIII and BamHI, thus to obtain a plasmid DNA for human preprorenin expression (PcDNA3.1(+)/hREN).

### Experimental Example 2

### Expression of Preprorenin and Purification of Prorenin (24-406)

Expression of human preprorenin was conducted using FreeStyle 293 Expression System (Invitrogen Corp.). According to the manual accompanying the FreeStyle 293 Expression System, the plasmid DNA for human preprorenin expression (pcDNA3.1(+)/hREN) constructed in Experimental Example 1 was used to conduct transient expression by FreeStyle 293-F cells. After transfection of the plasmid DNA, the cells were subjected to shaking culture under the conditions of 37° C., 8% CO₂ and 125 rpm for 3 days. A 600-ml aliquot of the culture solution was centrifuged at 2,000 rpm for 10 min to recover the culture supernatant containing prorenin (24-406). The culture supernatant was concentrated by ultrafiltration using a PM10 membrane (Millipore, Inc.) to a volume of about 50 ml, and then was dialyzed against 20 mM Tris-hydrochloric acid (pH 8.0). The dialyzate was fed to a 6-ml RESOURCE Q column (Amersham Biosciences, Inc.) equilibrated with 20 mM Tris-hydrochloric acid (pH 8.0) at a flow rate of 3 ml/min to adsorb the prorenin (24-406). After washing the column with the buffer solution used in the equilibration, elution was carried out by means of a linear concentration gradient of sodium chloride from 0 M to 0.4 M. The fraction containing prorenin (24-406) was collected and concentrated using Vivaspin 20 (molecular weight cut off 10,000; Vivascience, Inc.) to a volume of about 2 ml.

The concentrated liquid was subjected to gel filtration chromatography using HiLoad 16/60 Superdex 200 pg (Amersham Biosciences, Inc.) equilibrated with 20 mM Tris-hydrochloric acid (pH 8.0) containing 0.15 M sodium chloride, at a flow rate of 1.4 ml/min, thus to obtain 3.6 mg of purified prorenin (24-406).

### Experimental Example 3

### Purification of Active Type Renin (67-406)

To 3.6 mg of prorenin (24-406) was dissolved in 5.2 ml of 0.1 M Tris-hydrochloric acid (pH 8.0), 12 μg of trypsin (Roche Diagnostics Corp.) was added, and the mixture was allowed to react at 28° C. for 55 min to carry out activation of renin. After the reaction, 0.4 ml of immobilized trypsin inhibitor (Pierce Biotechnology, Inc.) was added to remove the trypsin used in the activation by adsorption. The reaction liquid containing the active type renin was concentrated using Vivaspin 20 (molcular weight cut off 10,000; Vivascience, Inc.), and was diluted with 20 mM Tris-hydrochloric acid (pH 8.0). The diluted liquid was fed to a TSKgel DEAE-5 PW column (7.5 mm I.D.×75 mm, Tosoh Corp.) equilibrated with 20 mM Tris-hydrochloric acid (pH 8.0) at a flow rate of 1 ml/min to adsorb the active type renin (67-406). The column was washed with the buffer solution used for the equilibration, and then elution was carried out by means of a sodium chloride linear concentration gradient from 0 M to 0.3 M, thus to obtain 1.5 mg of a purified product of active type renin (67-406).

### Experimental Example 4

### Measurement of Renin Inhibition Value

5 μl each of the test compound (containing 50% DMSO) was added to each well of a 384-well black plate (Nalge Nunc International Co., Ltd.). Renin was diluted with a buffer solution for reaction (20 mM citric acid-sodium citrate (pH 6.0)) to a concentration of 0.5 μg/ml, and 35 μl each of the dilution was added to each well. The dilution was left to stand at 37° C. for 10 min, and then 10 μl of each of a 25 μM solution of substrate peptide (FITC-Acp-Asp-Arg-Val-Tyr-Ile-His-Pro-Phe-His-Leu-Val-Ile-His-Asn-Arg-NH₂) was added to each well to initiate the reaction. The reaction mixture was left to stand at 37° C. for 30 min, and then 50 μl each of a reaction terminating solution [200 mM Tris-hydrochloric acid (pH 8.0), 0.04% Triton-X 100, 0.4% Coating 3 reagent (Caliper Life Sciences Corp.) and 1 μM CGP-29287 (Bachem Holding AG)] was added to each well to terminate the reaction.

The substrate peptide and the product peptide were separated by a microchip type capillary electrophoresis system 250HTS (Caliper Life Sciences Co., Ltd.), and the rate of reaction [(peak height of product)/(peak height of product+peak height of substrate)×100(%)] was calculated from the ratio of the respective peak height of the peptides obtained by fluorimetric detection (excitation wavelength 457 nm, measurement wavelength 530 nm), and was used as an index of the renin activity.

While the reaction rate of the well where 50% DMSO only was added was taken as 0% inhibition rate, and the reaction rate of the well where no enzyme Was added was taken as 100% inhibition rate, the renin inhibitory activity of the wells where the test compound (containing 50% DMSO) was added was calculated.

The results are presented in Table 16.

TABLE 16
Human Renin Inhibitory Activity

| | inhibitory activity | inhibitory activity |
|---|---|---|
| Ex. No. | (%) at 10 μM | (%) at 1 μM |
| 43 | 106 | 104 |
| 56 | 103 | 104 |
| 57 | 108 | 108 |
| 73 | 101 | 97 |
| 99 | 107 | 105 |
| 105 | 104 | 102 |
| 161 | 101 | 100 |
| 472 | 102 | 100 |
| 475 | 99 | 97 |
| 476 | 99 | 99 |
| 477 | 104 | 102 |
| 478 | 101 | 99 |

It can be seen from the results of Table 16 that compound (I) of the present invention has excellent renin inhibitory activity.

### INDUSTRIAL APPLICABILITY

The cyclic amine compound of the present invention has excellent renin inhibitory activity and thus is useful as an agent for the prophylaxis or treatment of hypertension, various organ damages attributable to hypertension, and the like.

This application is based on application No. 60/774,133 filed in USA, the contents of which are incorporated hereinto by reference.

1. A compound represented by the formula: 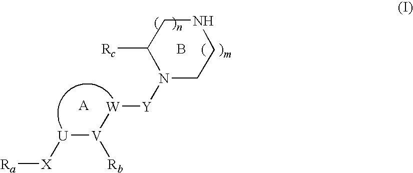
wherein
ring A is a 5- or 6-membered aromatic heterocycle optionally having substituent(s);
U, V and W are each independently C or N, provided that when any one of U, V and W is N, then the others should be C;
Ra and Rb are each independently a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
X is a bond, or a spacer having 1 to 6 atoms in the main chain;
Y is a spacer having 1 to 6 atoms in the main chain;
Rc is a hydrocarbon group optionally containing heteroatom(s) as the constituting atom(s), which optionally has substituent(s);
m and n are each independently 1 or 2; and
ring B optionally further has substituent(s),
or a salt thereof.

2. The compound of claim 1, wherein ring A is a 5-membered aromatic heterocycle optionally having substituent(s).

3. The compound of claim 1, wherein ring A is imidazole or pyrrole, each of which optionally has substituent(s).

4. The compound of claim 1, wherein Ra and Rb are each independently a cyclic group optionally having substituent(s).

5. The compound of claim 1, wherein Ra and Rb are each independently a C₆₋₁₄ aryl group optionally having substituent(s), a 5- or 6-membered non-aromatic heterocyclic group optionally having substituent(s), or a C₃₋₁₀ cycloalkyl group condensed with a benzene ring, which optionally has substituent(s).

6. The compound of claim 1, wherein Ra and Rb are each independently a C₆₋₁₄ aryl group optionally having substituent(s), or a C₃₋₁₀ cycloalkyl group condensed with a benzene ring, which optionally has substituent(s).

7. The compound of claim 1, wherein Ra is a phenyl group optionally having substituent(s), an indanyl group optionally having substituent(s) or a piperidinyl group optionally having substituent(s).

8. The compound of claim 1, wherein Rb is a phenyl group optionally having substituent(s).

9. The compound of claim 1, wherein X is a bond or a straight chain C₁₋₆ alkylene group optionally having substituent(s).

10. The compound of claim 1, wherein X is a bond, or a group represented by the formula: —(R¹)C(R²)— (wherein R¹ and R² are each independently a hydrogen atom or a C₁₋₃ alkyl group).

11. The compound of claim 1, wherein X is a bond.

12. The compound of claim 1, wherein Y is —CO—, —CH₂—, —CH₂CO— or —SO₂—.

13. The compound of claim 1, wherein Y is —CO—.

14. The compound of claim 1, wherein Rc is
1) a group represented by the formula:
R³—(Z₁)q-(Z)p-
wherein
R³ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₁ is —CO—, —O—, —S—, —S(O)— or —S(O)₂—; and
p and q are each independently 0 or 1;
2) a group represented by the formula:
R⁴—Z₂—(R⁵)C(R⁶)—(Z)p-
wherein
R⁴ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
R⁵ and R⁶ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s), or R⁵ and R⁶ in combination form an oxo group;
Z is a C₁₋₄ alkylene group;
Z₂ is —O—, or a group represented by the formula: —N(R⁷)— (wherein R⁷ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s));
p is 0 or 1; and
when Z₂ is a group represented by the formula: —N(R⁷)—, then R⁴ and R⁷ are optionally bonded to each other to form, together with the adjacent nitrogen atom, a nitrogen-containing heterocycle optionally having substituent(s);
3) a group represented by the formula:
R⁸—Z₃—N(R⁹)—(Z)p-
wherein
R⁸ and R⁹ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₃ is —CO—, —CONH— or —SO₂—; and
p is 0 or 1;
4) a group represented by the formula: 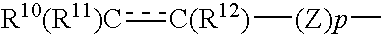
wherein
R¹⁰, R¹¹ and R¹² are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group; [symbol]
is a single bond or a double bond; and
p is 0 or 1; or
5) a group represented by the formula:
R¹³O—N═C(R¹⁴)—(Z)p-
wherein
R¹³ and R¹⁴ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group; and
p is 0 or 1.

15. The compound of claim 1, wherein Rc is a group represented by the formula:
R³—(Z₁)q-(Z)p-
wherein
R³ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z¹ is —CO—, —O—, —S—, —S(O)— or —S(O)₂—; and
p and q are each independently 0 or 1.

16. The compound of claim 1, wherein Rc is a group represented by the formula:
R⁴—Z₂—(R⁵)C(R⁶)—(Z)p-
wherein
R⁴ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
R⁵ and R⁶ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s), or R⁵ and R⁶ in combination form an oxo group;
Z is a C₁₋₄ alkylene group;
Z₂ is —O—, or a group represented by the formula: —N(R⁷)— (wherein R⁷ is a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s));
p is 0 or 1; and
when Z₂ is a group represented by the formula: —N(R⁷)—, then R⁴ and R⁷ are optionally bonded to each other to form, together with the adjacent nitrogen atom, a nitrogen-containing heterocycle optionally having substituent(s).

17. The compound of claim 1, wherein Rc is a group represented by the formula:
R⁸—Z₃—N(R⁹)—(Z)p-
wherein
R⁸ and R⁹ are each independently a hydrogen atom, a cyclic group optionally having substituent(s), a C₁₋₁₀ alkyl group optionally having substituent(s), a C₂₋₁₀ alkenyl group optionally having substituent(s), or a C₂₋₁₀ alkynyl group optionally having substituent(s);
Z is a C₁₋₄ alkylene group;
Z₃ is —CO—, —CONH— or —SO₂—; and
p is 0 or 1.

18. The compound of claim 1, wherein Rc is a C₁₋₆ alkyl group optionally substituted by 1 to 3 substituents selected from
(i) an optionally substituted C₆₋₁₄ aryl group, and
(ii) an optionally substituted C₁₋₆ alkoxy group.

19. The compound of claim 1, wherein both m and n are 1.

20. The compound of claim 1, wherein the compound represented by the formula (I) is a compound selected from the group consisting of
(2R)-2-benzyl-1-{[1-(2,3-dimethoxyphenyl)-5-methyl-2-phenyl-1H-pyrrol-3-yl]carbonyl}piperazine,
4-[3-(4-{[(2R)-2-benzylpiperazin-1-yl]carbonyl}-5-phenyl-1H-imidazol-1-yl)phenyl]morpholine,
(2R)-2-benzyl-1-{[1-(2,3-dimethoxyphenyl)-5-phenyl-1H-imidazol-4-yl]carbonyl}piperazine,
(2R)-2-benzyl-1-{[1-(2,3-dihydro-1H-inden-2-yl)-5-phenyl-1H-imidazol-4-yl]carbonyl}piperazine,
2-{3-[(2-benzylpiperazin-1-yl)carbonyl]-2-phenyl-1H-pyrrol-1-yl}-N-butylaniline,
4-[3-(3-{[(2R)-2-benzylpiperazin-1-yl]carbonyl}-5-methyl-2-phenyl-1H-pyrrol-1-yl)phenyl]morpholine,
4-[((2R)-1-{[1-(3-morpholinophenyl)-5-phenyl-1H-imidazol-4-yl]carbonyl}piperazin-2-yl)methyl]benzoic acid,
4-[3-(4-{[(2S)-2-({[4-(methylsulfonyl)benzyl]oxy}methyl)piperazin-1-yl]carbonyl}-5-phenyl-1H-imidazol-1-yl)phenyl]morpholine,
(2R)-2-benzyl-1-[(2-methoxy-1,5-diphenyl-1H-imidazol-4-yl)carbonyl]piperazine,
(2R)-2-benzyl-1-({5-phenyl-1-[1-(phenylsulfonyl)piperidin-3-yl]-1H-imidazol-4-yl}carbonyl)piperazine,
(2R)-2-benzyl-1-[(1-{1-[(6-methoxypyridin-3-yl)sulfonyl]piperidin-3-yl}-5-phenyl-1H-imidazol-4-yl)carbonyl]piperazine, and
4-[(3S)-3-(4-{[(2R)-2-benzylpiperazin-1-yl]carbonyl}-5-phenyl-1H-imidazol-1-yl)-5-phenylpentanoyl]morpholine.

21. A prodrug of the compound of claim 1.

22. A medicine comprising the compound of claim 1 or a salt thereof, or a prodrug thereof.

23. The medicine of claim 22, which is a renin inhibitory drug.

24. The medicine of claim 22, which is an agent for the prophylaxis or treatment of hypertension.

25. The medicine of claim 22, which is an agent for the prophylaxis or treatment of various organ damages attributable to hypertension.

26. A renin inhibitory drug comprising a compound represented by the formula: 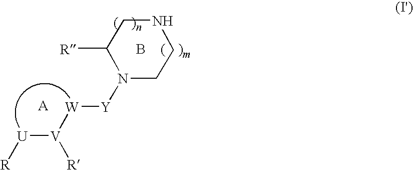
wherein
ring A is an aromatic heterocycle optionally having substituent(s);
U, V and W are each independently C or N, provided that when any one of U, V and W is N, then the others should be C;
R, R′ and R″ are each independently a substituent;
Y is a spacer having 1 to 6 atoms in the main chain;
m and n are each independently 1 or 2; and
ring B optionally further has substituent(s),
or a salt thereof, or a prodrug thereof.
